(19) United States
(12) Patent Application Publication
(10) Pub. No.: US 2004/0014053 A1
(43) Pub. Date: Jan. 22, 2004

(54) Novel proteins and nucleic acids encoding same

(76) Inventors: Bryan D. Zerhusen, Branford, CT (US); Meera Patturajan, Branford, CT (US); Ramesh Kekuda, Norwalk, CT (US); Charles E. Miller, Guilford, CT (US); Daniel K. Rieger, Branford, CT (US); Carol E.A. Pena, New Haven, CT (US); Richard A. Shimkets, Guilford, CT (US); Li Li, Branford, CT (US); Constance Berghs, New Haven, CT (US); Mei Zhong, Branford, CT (US); Stacie J. Casman, North Haven, CT (US); Edward Z. Voss, Wallingford, CT (US); Ferenc L. Boldog, North Haven, CT (US); Muralidhara Padigaru, Branford, CT (US); Glennda Smithson, Guilford, CT (US); Weizhen Ji, Branford, CT (US); Linda Gorman, Branford, CT (US); Corine A.M. Vernet, Branford, CT (US); Mario W. Leite, Milford, CT (US); Xiaojia Sasha Guo, Branford, CT (US); David W. Anderson, Branford, CT (US); Kimberly A. Spytek, New Haven, CT (US); Valerie Gerlach, Branford, CT (US); Catherine E. Burgess, Wethersfield, CT (US); Nikolai V. Khramtsov, Branford, CT (US); Tatiana Ort, Milford, CT (US); Karen Ellerman, Branford, CT (US); Luca Rastelli, Guilford, CT (US); Michele L. Agee, Wallingford, CT (US); Amitabha Chaudhuri, Madison, CT (US); John S. Chant, Branford, CT (US); Vincent A. DiPippo, East Haven, CT (US); Shlomit R. Edinger, New Haven, CT (US); Andrew J. Eisen, Rockville, MD (US); Esha A. Gangolli, Madison, CT (US); Loic Giot, Madison, CT (US); Chean Eng Ooi, Branford, CT (US); Mark E. Rothenberg, Clinton, CT (US); Steven K. Spaderna, Berlin, CT (US); Tord Hjalt, Lomma (SE); Xiaohong Liu, Lexington, MA (US); Raymond J. Taupier JR., East Haven, CT (US); Elina Catterton, Madison, CT (US); Suresh G. Shenoy, Branford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/210,130

(22) Filed: Aug. 1, 2002

Related U.S. Application Data
(60) Provisional application No. 60/309,501, filed on Aug. 2, 2001.
(60) Provisional application No. 60/316,508, filed on Aug. 31, 2001.
(60) Provisional application No. 60/354,655, filed on Feb. 5, 2002.
(60) Provisional application No. 60/310,291, filed on Aug. 3, 2001.
(60) Provisional application No. 60/383,887, filed on May 29, 2002.
(60) Provisional application No. 60/310,951, filed on Aug. 8, 2001.
(60) Provisional application No. 60/323,936, filed on Sep. 21, 2001.
(60) Provisional application No. 60/381,039, filed on May 16, 2002.
(60) Provisional application No. 60/311,292, filed on Aug. 9, 2001.
(60) Provisional application No. 60/311,979, filed on Aug. 13, 2001.
(60) Provisional application No. 60/312,203, filed on Aug. 14, 2001.
(60) Provisional application No. 60/361,764, filed on Mar. 5, 2002.
(60) Provisional application No. 60/313,201, filed on Aug. 17, 2001.
(60) Provisional application No. 60/338,078, filed on Dec. 3, 2001.
(60) Provisional application No. 60/380,971, filed on May 15, 2002.
(60) Provisional application No. 60/313,156, filed on Aug. 17, 2001.
(60) Provisional application No. 60/313,702, filed on Aug. 20, 2001.
(60) Provisional application No. 60/380,980, filed on May 15, 2002.
(60) Provisional application No. 60/313,643, filed on Aug. 20, 2001.
(60) Provisional application No. 60/383,761, filed on May 28, 2002.
(60) Provisional application No. 60/322,716, filed on Sep. 17, 2001.
(60) Provisional application No. 60/314,031, filed on Aug. 21, 2001.
(60) Provisional application No. 60/314,466, filed on Aug. 23, 2001.
(60) Provisional application No. 60/315,403, filed on Aug. 28, 2001.
(60) Provisional application No. 60/315,853, filed on Aug. 29, 2001.
(60) Provisional application No. 60/373,825, filed on Apr. 19, 2002.

(51) Int. Cl. C12Q 1/68; G01N 33/574; C07H 21/04; C12N 9/00; C12P 21/02; C12N 5/06; C07K 14/47

(52) U.S. Cl. 435/6; 435/7.23; 435/69.1; 435/183; 435/320.1; 435/325; 530/350; 536/23.2

(57) ABSTRACT

The present invention provides novel isolated polynucleotides and small molecule target polypeptides encoded by the polynucleotides. Antibodies that immunospecifically bind to a novel small molecule target polypeptide or any derivative, variant, mutant or fragment of that polypeptide, polynucleotide or antibody are disclosed, as are methods in which the small molecule target polypeptide, polynucleotide and antibody are utilized in the detection and treatment of a broad range of pathological states. More specifically, the present invention discloses methods of using recombinantly expressed and/or endogenously expressed proteins in various screening procedures for the purpose of identifying therapeutic antibodies and therapeutic small molecules associated with diseases. The invention further discloses therapeutic, diagnostic and research methods for diagnosis, treatment, and prevention of disorders involving any one of these novel human nucleic acids and proteins.

### RELATED APPLICATIONS

This application claims priority to provisional patent applications U.S. Serial Nos. 60/309,501, filed on Aug. 2, 2001; 60/316,508, filed on Aug. 31, 2001; 60/354,655, filed on Feb. 5, 2002; 60/3 10,291, filed on Aug. 3, 2001; 60/383,887, filed on May 29, 2002; 60/310,951, filed on Aug. 8, 2001; 60/323,936, filed on Sep. 21, 2001; 60/381,039, filed on May 16, 2002; 60/311,292, filed on Aug. 9, 2001; 60/311,979, filed on Aug. 13, 2001; 60/312,203, filed on Aug. 14, 2001; 60/361,764, filed on Mar. 5, 2002; 60/313,201, filed on Aug. 17, 2001; 60/338,078, filed on Dec. 3, 2001; 60/380,971, filed on May 15, 2002; 60/313,156, filed on Aug. 17, 2001; 60/313,702, filed on Aug. 20, 2001; 60/380,980, filed on May 15, 2002; 60/313,643, filed on Aug. 20, 2001; 60/383,761, filed on May 28, 2002; 60/322,716, filed on Sep. 17, 2001; 60/314,031, filed on Aug. 21, 2001, 60/314,466, filed on Aug. 23, 2001; 60/315,403, filed on Aug. 28, 2001; 60/315,853, filed on Aug. 29, 2001, 60/373,825, filed on Apr. 19, 2002; each of which is incorporated herein by reference in its entirety.

### FIELD OF THE INVENTION

The present invention relates to novel polypeptides that are targets of small molecule drugs and that have properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways involve extracellular signaling proteins, cellular receptors that bind the signaling proteins and signal transducing components located within the cells.

Signaling proteins may be classified as endocrine effectors, paracrine effectors or autocrine effectors. Endocrine effectors are signaling molecules secreted by a (given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding, process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Small molecule targets have been implicated in various disease states or pathologies. These targets may be proteins, and particularly enzymatic proteins, which are acted upon by small molecule drugs for the purpose of altering target function and achieving a desired result. Cellular, animal and clinical studies can be performed to elucidate the genetic contribution to the etiology and pathogeniesis of conditions in which small molecule targets are implicated in a variety of physiologic, pharmicologic or native states. These studies utilize the core technologies at CuraGen Corporation to look at differential gene expression, protein-protein interactions, large-scale sequencing of expressed genes and the association of genetic variations such as, but not limited to, single nucleotide polymorphisms (SNPs) or splice variants in and between biological samples from experimental and control groups. The goal of such studies is to identify potential avenues for therapeutic intervention in order to prevent, treat the consequences or cure the conditions.

In order to treat diseases, pathologies and other abnormal states or conditions in which a mammalian organism has been diagnosed as being, or as being at risk for becoming, other than in a normal state or condition, it is important to identify new therapeutic agents. Such a procedure includes at least the steps of identifying a target component within an affected tissue or organ, and identifying a candidate therapeutic agent that modulates the functional attributes of the target. The target component may be any biological macromolecule implicated in the disease or pathology. Commonly the target is a polypeptide or protein with specific functional attributes. Other classes of macromolecule may be a nucleic acid, a polysaccharide, a lipid such as a complex lipid or a glycolipid; in addition a target may be a sub-cellular structure or extra-cellular structure that is comprised of more than one of these classes of macromolecule. Once such a target has been identified, it may be employed in a screening assay in order to identify favorable candidate therapeutic agents from among a large population of substances or compounds.

In many cases the objective of such screening assays is to identify small molecule candidates; this is commonly approached by the use of combinatorial methodologies to develop the population of substances to be tested. The implementation of high throughput screening methodologies is advantageous when working with large, combinatorial libraries of compounds.

### SUMMARY OF THE INVENTION

The invention includes nucleic acid sequences and the novel polypeptides they encode. The novel nucleic acids and polypeptides are referred to herein as NOVX, or NOV1, NOV2. NOV3, etc., nucleic acids and polypeptides. These nucleic acids and polypeptides, as well as derivatives, homologs, analogs and fragments thereof, will hereinafter be collectively designated as “NOVX” nucleic acid, which represents the nucleotide sequence selected from the group consisting of SEQ ID NO: 2n−1, wherein n is an integer,between 1 and 88, or polypeptide sequences, which represents the group consisting of SEQ ID NO: 2n, wherein n is an integer between 1 and 88.

In one aspect, the invention provides an isolated polypeptide comprising a mature form of a NOVX amino acid. One example is a variant of a mature form of a NOVX amino acid sequence wherein any amino acid in the mature form is changed to a different amino acid, provided that no more than 15% of tlhe amino acid residues in the sequence of the mature form are so changed. The amino acid can be, for example, a NOVX amino acid sequence or a variant of a NOVX amino acid sequence, wherein any amino acid specified in the chosen sequence is changed to a different amino acid, provided that no more than 15% of the amino acid residues in the sequence are so changed. The invention also includes fragments of any of these. In another aspect, the invention also includes an isolated nucleic acid that encodes a NOVX polypeptide, or a fragment, homolog, analog or derivative thereof.

Also included in the invention is a NOVX polypeptide that is a naturally occurring allelic variant of a NOVX sequence. In one embodiment, the allelic variant includes an amino acid sequence that is the translation of a nucleic acid sequence differing by a single nucleotide from a NOVX nucleic acid sequence. In another embodiment, the NOVX polypeptide is a variant polypeptide described therein, wherein any amino acid specified in the chosen sequence is changed to provide a conservative substitution. In one embodiment, the invention discloses a method for determining the presence or amount of the NOVX polypeptide in a sample. The method involves the steps of: providing a sample; introducing the sample to an antibody that binds immunospecifically to the polypeptide; and determining the presence or amount of antibody bound to the NOVX polypeptide, thereby determining the presence or amount of the NOVX polypeptide in the sample. In another embodiment, the invention provides a method for determining the presence of or predisposition to a disease associated with altered levels of a NOVX polypeptide in a mammalian subject. This method involves the steps of: measuring the level of expression of the polypeptide in a sample from the first mammalian subject; and comparing the amount of the polypeptide in the sample of the first step to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In a further embodiment, the invention includes a method of identifying an agent that binds to a NOVX polypeptide. This method involves the steps of: introducing the polypeptide to the agent; and determining whether the agent binds to the polypeptide. In various embodiments, the agent is a cellular receptor or a downstream effector.

In another aspect, the invention provides a method for identifying a potential therapeutic agent for use in treatment of a pathology, wherein the pathology is related to aberrant expression or aberrant physiological interactions of a NOVX polypeptide. The method involves the steps of: providing a cell expressing the NOVX polypeptide and having a property or function ascribable to the polypeptide; contacting the cell with a composition comprising a candidate substance: and determining whether the substance alters the property or function ascribable to the polypeptide; whereby, if an alteration observed in the presence of the substance is not observed when the cell is contacted with a composition devoid of the substance, the substance is identified as a potential therapeutic agent. In another aspect, the invention describes a method for screening for a modulator of activity or of latency or predisposition to a pathology associated with the NOVX polypeptide. This method involves the following steps: administering a test compound to a test animal at increased risk for a pathology associated with the NOVX polypeptide, wherein the test animal recombinantly expresses the NOVX polypeptide. This method involves the steps of measuring the activity of the NOVX polypeptide in the test animal after administering the compound of step; and comparing the activity of the protein in the test animal with the activity of the NOVX polypeptide in a control animal not administered the polypeptide, wherein a change in the activity of the NOVX polypeptide in the test animal relative to the control animal indicates the test compound is a modulator of latency of, or predisposition to, a pathology associated with the NOVX polypeptide. In one embodiment, the test animal is a recombinant test animal that expresses a test protein transgene or expresses the transgene under the control of a promoter at an increased level relative to a wild-type test animal, and wherein the promoter is not the native gene promoter of the transgene. In another aspect, the invention includes a method for modulating the activity of the NOVX polypeptide, the method comprising introducing a cell sample expressing the NOVX polypeptide with a compound that binds to the polypeptide in an amount sufficient to modulate the activity of the polypeptide.

The invention also includes an isolated nucleic acid that encodes a NOVX polypeptide, or a fragment, homolog, analog or derivative thereof. In a preferred embodiment, the nucleic acid molecule comprises the nucleotide sequence of a naturally occurring allelic nucleic acid variant. In another embodiment, the nucleic acid encodes a variant polypeptide, wherein the variant polypeptide has the polypeptide sequence of a naturally occurring polypeptide variant. In another embodiment, the nucleic acid molecule differs by a single nucleotide from a NOVX nucleic acid sequence. In one embodiment, the NOVX nucleic acid molecule hybridizes under stringent conditions to the nucleotide sequence selected from the group consisting of SEQ ID NO: 2n−1, wherein n is an integer between 1 and 88, or a complement of the nucleotide sequence. In another aspect, the invention provides a vector or a cell expressing a NOVX nucleotide sequence.

In one embodiment, the invention discloses a method for modulating the activity of a NOVX polypeptide. The method includes the steps of: introducing a cell sample expressing the NOVX polypeptide with a Compound that binds to the polypeptide in an amount sufficient to modulate the activity of the polypeptide. In another embodiment, the invention includes an isolated NOVX nucleic acid molecule comprising a nucleic acid sequence encoding a polypeptide comprising a NOVX amino acid sequence or a variant of a mature form of the NOVX amino acid sequence, wherein any amino acid in the mature form of the chosen sequence is changed to a different amino acid, provided that no more than 15% of the amino acid residues in the sequence of the mature form are so changed. In another embodiment, the invention includes an amino acid sequence that is a variant of the NOVX amino acid sequence, in which any amino acid specified in the chosen sequence is changed to a different amino acid, provided that no more than 15% of the amino acid residues in the sequence are so changed.

In one embodiment, the invention discloses a NOVX nucleic acid fragment encoding at least a portion of a NOVX polypeptide or any variant of the polypeptide, wherein any amino acid of the chosen sequence is changed to a different amino acid, provided that no more than 10% of the amino acid residues in the sequence are so changed. In another embodiment, the invention includes the complement of any of the NOVX nucleic acid molecules or a naturally occurring allelic nucleic acid variant. In another embodiment, the invention discloses a NOVX nucleic acid molecule that encodes a variant polypeptide, wherein the variant polypeptide has the polypeptide sequence of a naturally occurring polypeptide variant. In another embodiment, the invention discloses a NOVX nucleic acid, wherein the nucleic acid molecule differs by a single nucleotide from a NOVX nucleic acid sequence.

In another aspect, the invention includes a NOVX nucleic acid, wherein one or more nucleotides in the NOVX nucleotide sequence is changed to a different nucleotide provided that no more than 15% of the nucleotides are so changed. In one embodiment, the invention discloses a nucleic acid fragment of the NOVX nucleotide sequence and a nucleic acid fragment wherein one or more nucleotides in the NOVX nucleotide sequence is changed from that selected from the group consisting of the chosen sequence to a different nucleotide provided that no more than 15% of the nucleotides are so changed. In another embodiment, the invention includes a nucleic acid molecule wherein the nucleic acid molecule hybridizes under stringent conditions to a NOVX nucleotide sequence or a complement of the NOVX nucleotide sequence. In one embodiment, the invention includes a nucleic acid molecule, wherein the sequence is changed such that no more than 15% of the nucleotides in the coding sequence differ from the NOVX nucleotide sequence or a fragment thereof.

In a further aspect, the invention includes a method for determining the presence or amount of the NOVX nucleic acid in a sample. The method involves the steps of: providing the sample; introducing the sample to a probe that binds to the nucleic acid molecule; and determining the presence or amount of the probe bound to the NOVX nucleic acid molecule, thereby determining the presence or amount of the NOVX nucleic acid molecule in the sample. In one embodiment, the presence or amount of the nucleic acid molecule is used as a marker for cell or tissue type.

In another aspect, the invention discloses a method for determining the presence of or predisposition to a disease associated with altered levels of the NOVX nucleic acid molecule of in a first mammalian subject. The method involves the steps of: measuring the amount of NOVX nucleic acid in a sample from the first mammalian subject; and comparing the amount of the nucleic acid in the sample of step (a) to the amount of NOVX nucleic acid present in a control sample from a second mammalian subject known not to have or not be predisposed to, the disease; wherein an alteration in the level of the nucleic acid in the first subject as compared to the control sample indicates the presence of or predisposition to the disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ | SEQ ID | |
|---|---|---|---|---|
| | | ID NO | NO | |
| NOVX | Internal | (nucleic | (amino | |
| Assignment | Identification | acid) | acid) | Homology |
| 1a | CG102071-03 | 1 | 2 | MAP Kinase Phosphatase-like protein |
| 2a | CG102734-01 | 3 | 4 | Rab4-like protein |
| 2b | CG102734-02 | 5 | 6 | RAS-Related protein RAB-like protein |
| 2c | 209829447 | 7 | 8 | Rab4-like protein |
| 3a | CG112785-01 | 9 | 10 | GPCR-like protein |
| 4a | CG116818-02 | 11 | 12 | pyruvate carboxylase isoform-like protein |
| 5a | CG117653-02 | 13 | 14 | ATP binding cassette ABCG1-like protein |
| 6a | CG119674-02 | 15 | 16 | Orphan Neurotransmitter Transporter NTT5-like |
| | | | | protein |
| 6b | CG119674-03 | 17 | 18 | Orphan Neurotransmitter Transporter NTT5-like |
| | | | | protein |
| 7a | CG120123-02 | 19 | 20 | Amino acid transporter ATA2-like protein |
| 8a | CC120814-01 | 21 | 22 | Glutathione S-transferase-like protein |
| 9a | CG122768-01 | 23 | 24 | Peroxiredoxin 2 like protein-like protein |
| 10a | CG122786-01 | 25 | 26 | Prostaglandin F Synthase-like protein |
| 11a | CG122795-01 | 27 | 28 | Serine/Threonine Protein Phosphatase-like protein |
| 12a | CG122805-01 | 29 | 30 | Ubiquinol-cytochrome C reductase hinge protein |
| | | | | like-protein |
| 13a | CG123100-01 | 31 | 32 | Mitogen activated kinase-like protein |
| 14a | CG124136-01 | 33 | 34 | Striated muscle-specific Serine/Threonine Protein |
| | | | | kinase-like protein |
| 14b | CG124136-02 | 35 | 36 | Striated Muscle-Specific Serine/Threonine Protein |
| | | | | Kinase-like protein |
| 14c | CG124136-03 | 37 | 38 | Striated muscle-specific Serine/Threonine Protein |
| | | | | kinase-like protein |
| 14d | 283022671 | 39 | 40 | Striated muscle-specific Serine/Threonine Protein |
| | | | | kinase-like protein |
| 15a | CG124553-01 | 41 | 42 | Polypeptide N-acetylgalactosaminyltransferase- |
| | | | | like protein |
| 15b | 276644723 | 43 | 44 | Polypeptide N-acetylgalactosaminyltransferase- |
| | | | | like protein |
| 15c | 276644750 | 45 | 46 | Polypeptide N-acetylgalactosaminyltransferase- |
| | | | | like protein |
| 16a | CG124691-01 | 47 | 48 | Polypeptide N-Acetylgalactosaminyltransferase- |
| | | | | like protein |
| 16b | CG124691-01 | 49 | 50 | Polypeptide N-Acetylgalactosaminyltransferase- |
| | | | | like protein (taqman panel) |
| 16c | CG124691-01 | 51 | 52 | UDP-GalNAc Transferase-like protein |
| 17a | CG125169-01 | 53 | 54 | Alcohol Dehydrogenase Class III CHI Chain-like |
| | | | | protein |
| 18a | CG125197-01 | 55 | 56 | Lysophospholipase (Acyl-Protein Thioesterase-1)- |
| | | | | like protein |
| 19a | CG125215-01 | 57 | 58 | AMP-binding enzyme-like protein |
| 19b | CG-125215-02 | 59 | 60 | AMP-binding enzyme-like Protein |
| 20a | CG125332-02 | 61 | 62 | natriuretic peptide-converting enzyme-like protein |
| 21a | CG125363-01 | 63 | 64 | Mitogen-activated protein kinase-like protein |
| 22a | CG126012-01 | 65 | 66 | Zinc transporter-like protein |
| 23a | CG126481-01 | 67 | 68 | Phosphodiesterase Hydrolase-like protein |
| 23b | CG126481-02 | 69 | 70 | Phosphodiesterase Hydrolase-like protein |
| 23c | 278459554 | 71 | 72 | Phosphodiesterase Hydrolase-like protein |
| 23d | 278463211 | 73 | 74 | Phosphodiesterase Hydrolase-like protein |
| 23e | 278465805 | 75 | 76 | Phosphodiesterase Hydrolase-like protein |
| 24a | CG127851-01 | 77 | 78 | Aldose 1-epimerase-like protein |
| 24b | CG127851-02 | 79 | 80 | Aldose 1-epimerase-like protein |
| 25a | CG127906-01 | 81 | 82 | Protease-like protein |
| 26a | CG128021-01 | 83 | 84 | Ubiquitin carboxyl-terminal hydrolase 11-like |
| | | | | protein |
| 27a | CG128291-01 | 85 | 86 | Matrix metalloproteinase 19-like protein |
| 28a | CG128380-01 | 87 | 88 | Calpain family cysteine protease-like protein |
| 29a | CG128439-02 | 89 | 90 | Endothelial Lipase-like protein |
| 29b | 171826603 | 91 | 92 | Endothelial Lipase-like protein |
| 30a | CG128489-01 | 93 | 94 | Thyroid peroxidase precursor-like protein |
| 31a | CG128825-01 | 95 | 96 | Tyrosine-protein kinase receptor FLT3-like |
| | | | | protein |
| 31b | CG128825-02 | 97 | 98 | Splice Variant of Tyrosine-protein kinase receptor |
| | | | | FLT3-like Proteins |
| 32a | CG128891-01 | 99 | 100 | Myotonic dystrophy kinase-related Cdc42-binding |
| | | | | kinase (MRCK)-like protein |
| 32b | CG128891-02 | 101 | 102 | Myotonic dystrophy kinase-related Cdc42-related |
| | | | | Kinase-like protein |
| 32c | 276585662 | 103 | 104 | IFC-Myotonic dystrophy kinase-related Cdc42- |
| | | | | related kinase-like protein |
| 33a | CG131490-01 | 105 | 106 | HEXOKINASE 1-like protein |
| 33b | CG131490-02 | 107 | 108 | hexokinase 1-like protein splice variant |
| 34a | CG131881-01 | 109 | 110 | Biphenyl-hydrolase Related Protein-like protein |
| 34b | CG131881-03 | 111 | 112 | Biphenyl-hydrolase Related Protein like protein |
| 34c | CG131881-04 | 113 | 114 | Biphenyl-hydrolase Related Protein-like protein |
| 34d | CG131881-05 | 115 | 116 | Biphenyl-hydrolase Related Protein-like protein |
| 35a | CG133535-01 | 117 | 118 | Tubulin-Tyrosine Ligase-like protein |
| 36a | CG133558-01 | 119 | 120 | Dipeptidyl Aminopeptidase Protein 6 (KIAA1492)- |
| | | | | like protein |
| 37a | CG133589-01 | 121 | 122 | ADAM-like protein |
| 37b | CG133589-02 | 123 | 124 | ADAM-like protein |
| 38a | CG133668-01 | 125 | 126 | Ras-related protein-like protein |
| 38b | CG133668-02 | 127 | 128 | Ras-related protein-like protein |
| 39a | CG133750-01 | 129 | 130 | Mixed lineage kinase MLK1-like protein |
| 40a | CG133819-01 | 131 | 132 | phosplipid-transporting ATPase VB-like |
| | | | | protein |
| 41a | CG134375-01 | 133 | 134 | peptidylprolyl isomerase A (Cyclophilin A)-like |
| | | | | protein |
| 42a | CG135546-01 | 135 | 136 | Adenylate kinase-like protein |
| 43a | CG136321-01 | 137 | 138 | Phosphatidylinositol-specific phospholipase-like |
| | | | | protein |
| 44a | CG136648-01 | 139 | 140 | Divalent cation transporter-like protein |
| 45a | CG54479-01 | 141 | 142 | Hepatocyte growth factor-like protein precursor |
| | | | | (MSP-like protein) |
| 45b | CG4479-02 | 143 | 144 | Hepatocyte growth factor-like protein precursor |
| | | | | (MSP-like protein) |
| 45c | CG54479-03 | 145 | 146 | Hepatocyte growth factor-like protein precursor |
| | | | | (MSP-like protein) |
| 45d | CG54479-04 | 147 | 148 | Hepatocyte growth factor-like protein precursor |
| | | | | (MSP-like protein) |
| 45e | CG54479-05 | 149 | 150 | Hepatocyte growth factor-like protein precursor |
| | | | | (MSP-like protein) |
| 45f | CG54479-06 | 151 | 152 | Hepatocyte growth factor-like protein precursor |
| | | | | (MSP-like protein) |
| 46a | CG56649-01 | 153 | 154 | Human membrane-type serine protease 6 (MTSP- |
| | | | | 6)-like protein |
| 46b | 169427553 | 155 | 156 | Human membrane-type serine protease 6 (MTSP- |
| | | | | 6)-like protein |
| 47a | CG57209-01 | 157 | 158 | Human EMR1 hormone receptor-like protein |
| 47b | CG57209-04 | 159 | 160 | Human EMR1 hormone receptor-like protein |
| 47c | 165275217 | 161 | 162 | Human EMR1 hormone receptor-like protein |
| 48a | CG59325-01 | 163 | 164 | Human endometrial cancer related protein, AXL- |
| | | | | like protein |
| 48b | CG59325-03 | 165 | 166 | Human endometrial cancer related protein, AXL- |
| | | | | like protein |
| 48c | CG59325-04 | 167 | 168 | AXL-receptor tyrosine Kinase-like protein |
| 48d | 172557413 | 169 | 170 | Human axl receptor-like protein |
| 48e | 172557493 | 171 | 172 | Human axl receptor-like protein |
| 48f | 172557606 | 173 | 174 | Human axl receptor-like protein |
| 49a | CG59582-03 | 175 | 176 | Red Cell Acid Phosphatase 1-like protein |

Table A indicates the homology of NOVX polypeptides to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

Pathologies, diseases, disorders and condition and the like that are associated with NOVX sequences include, but are not limited to: e.g., cardiomyopathy, atherosclerosis, hypertension, congenital heart defects, aortic stenosis, atrial septal defect (ASD), atrioventricular (A-V) canal defect, ductus arteriosus, pulmonary steniosis, subaortic stenosis, ventricular septal defect (VSD), valve diseases, tuberous sclerosis, scleroderma, obesity, metabolic disturbances associated with obesity, transplantation, adrenoleukodystrophy, congenital adrenal hyperplasia, prostate cancer, diabetes, metabolic disorders. neoplasm; adenocarcinoma, lymphoma, uterus cancer, fertility, hemophilia, hypercoagulation, idiopathic thorombocytopenic purpura, immunodeficienicies, graft versus host disease, AIDS, bronchial asthma, Crohn's disease: multiple sclerosis, treatment of Albright Hereditary Ostoedystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias,] of the metabolic syndrome X and wasting disorders associated with chronic diseases and various cancers, as shell as conditions such as transplantation and fertility.]

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g. detection of a variety of cancers.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identity proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 88; (b) a variant of a mature form of the amino acid sequence selected from the group consisting of SEQ ID NO: 2n, wherein n is an integer between 1 and 88, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 88; (d) a variant of the amino acid sequence selected from the group consisting of SEQ ID NO: 2n, wherein n is an integer between 1 and 88 wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 88; (b) a variant of a mature form of the amino acid sequence selected from the group consisting of SEQ ID NO: 2n, wherein n is an integer between 1 and 88 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 88; (d) a variant of the amino acid sequence selected from the group consisting of SEQ ID NO: 2n, wherein n is an integer between 1 and 88, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 88 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n−1, wherein n is an integer between 1 and 88; (b) a nucleotide sequence wherein one or more nucleotides in the nucleotide sequence selected from the group consisting of SEQ ID NO: 2n−1, wherein n is an integer between 1 and 88 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n−1, wherein n is an integer between 1 and 88; and (d) a nucleic acid fragment wherein one or more nucleotides in the nucleotide sequence selected from the group consisting of SEQ ID NO: 2n−1, wherein n is an integer between 1 and 88 is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e g A NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e g., host cell) in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-stranded or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NO: 2n−1, wherein n is an integer between 1 and 88, or a complement of this nucleotide sequence, can be isolated using standard molecular biology techniques and the sequence information provided herein. Using all or a portion of the nucleic acid sequence of SEQ ID NO: 2n−1, wherein n is an integer between 1 and 88, as a hybridization probe, NOVX molecules can be isolated using standard hybridization and cloning techniques (e.g., as described in Sambrook, et. al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y. 1993.)

A nucleic acid of the invention can be amplified using cDNA, mRNA or alternatively, genomic DNA, as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term oligonucleotide refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having( about 10 nt. 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO: 2n−1, wherein n is an integer between 1 and 88, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO: 2n−1, wherein n is an integer between 1 and 88, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO: 2n−1, wherein n is an integer between 1 and 88, is one that is sufficiently complementary to the nucleotide sequence of SEQ ID NO: 2n−1, wherein n is an integer between 1 and 88. that it can hydrogen bond with few or no mismatches to the nucleotide sequence shown in SEQ ID NO: 2n−1, wherein i? is an integer between 1 and 88, thereby forming a stable duplex.

As used herein, the term “complementary” refers to Watson−Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non−ionic, van der Waals, hydrophobic interactions, and the like. A physical interaction can be either direct or indirect. Indirect interactions may be through, or due to the effects of another polypeptide or compound. Direct binding refers to interactions that do not take place through, or due to, the effect of another polypeptide or Compound, but instead are without other substantial chemical intermediates.

“fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g. they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat, cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein, homologous nucleic acid sequences include those nucleic acid sequences that encode conservative amino acid substitutions (see below) in SEQ ID NO: 2n−1, wherein n is an integer between 1 and 88, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of tile three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e g., a stretch of DNA that would encode a protein of 50 amino acids or more.

The nucleotide sequences determined from the cloning of the human NOVX genes allows for the generation of probes and primers designed for use in identifying and/or cloning NOVX homologous in other cell types, e.g. from other tissues, as well as NOVX homologous from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO: 2n−1, wherein n is an integer between 1 and 88; or an anti-sense strand nucleotide sequence of SEQ ID NO: 2,n−1, wherein n is an integer between 1 and 88; or of a naturally occurring mutant of SEQ ID NO: 2n−1, wherein n is an integer between 1 and 88.

Probes based on the human NOVX nucleotide sequences can be used to detect transcripts or genomic sequences encoding the same or homologous proteins. In various 1 5 embodiments, the probe has a detectable label attached. e.g., the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO: 2,n−1, wherein n is an integer between 1 and 88, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences of SEQ ID NO: 2n−1, wherein n is an integer between 1 and 88, due to degeneracy of the genetic code and thus encode the same NOVX proteins as that encoded by the nucleotide sequences of SEQ ID NO: 2n−l, wherein n is an integer between 1 and 88. In another embodiment, an isolated nucleic acid molecule of the invention has a nucleotide sequence encoding a protein having an amino acid sequence of SEQ ID NO: 2n, wherein n is an integer between 1 and 88.

In addition to the human NOVX nucleotide sequences of SEQ ID NO: 2n−1 wherein n is an integer between 1 and 88, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “genie” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO: 2n−1, wherein n is an integer between 1 and 88. are intended to be within the scope of the invention. Nucleic acid molecules corresponding to natural allelic variants and homologous of the NOVX cDNAs of the invention call be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO: 2n−1 , wherein n is an integer between 1 and 88. In another embodiment, the nucleic acid is at least 10, 25, 50, 100, 250, 500, 750, 1000, 1500, or 2000 or more nucleotides in length. In yet another 30 embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes arc occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel. et. al., (eds.), CURRENT PROTOCOLS IN MOLECULAR BIOLOGY, John Wiley & Sons, N.Y. (1989), 6.3.1-6.3.6. Preferably, the conditions arc such that sequences at least about 65%. 70%, 75%, 85%, 90%, 95%, 98%. or 99% homologous to each other typically remain hybridized to each other. A non-limiting example of stringent hybridization conditions are hybridization in a high salt buffer comiiprising 6×SSC, 50 miiM Tris-HCl (pH 7.5), 1 mM EDTA, 0.02% PVP, 0.02% Ficoll, 0.02% BSA, and 500 mg/mil denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NO: 2n−1, wherein n is an integer between 1 and 88, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 2n−1, wherein n is an integer between 1 and 88, or fragments, analogs or derivatives thereof, under conditions of moderate stringency is provided. A non-limiting example of moderate stringency hybridization conditions are hybridization in 6×SSC, 5× Reinhardt's solution, 0.5% SDS and 100 mg/ml denatured salmon sperm DNA at 55 ° C., followed by one or more washes in 5×SSC, 0.1% SDS at 37° C. Other conditions of moderate stringency that may be used are well-known within the art. See, e g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NO: 2n−1, wherein n is an integer between 1 and 88. or fragments, analogs or derivatives thereof, under conditions of low stringency, is provided. A non-limiting example of low stringency hybridization conditions are hybridization in 35% formiamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextrani sulfate at 40° C. followed by one or more washes in 2×SSC, 25 mM Tris-HCl (pH 7.4), 5 mM EDTA, and 0.1% SDS at 50° C. Other conditions of low stringcncy that may be used are well known in the art e.g., as employed for cross-species hybridizations). See, e.g., Ansubel. et. al., (eds.), 1993, CURRENT PROTOCOLS IN MOLECULAR BIOLOGY, John Wiley & Sons, NY, and Kreigler, 1990. GENE TRANSFER AND EXPRESSION. A LABORATORY MANUAL. Stockton Stress, NY; Shilo and Weinberg, 1981, Proc. Natl. Acad. Sci. U.S.A. 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO: 2n−1, wherein n is an integer between 1 and 88, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO: 2n, wherein n is an integer between 1 and 88. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amendable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO: 2n−l, wherein n is an integer between 1 and 88, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NO: 2n, wherein n is an integer between 1 and 88. Preferably, the protein encoded by the nucleic acid molecule is at least about 60% homologous to SEQ ID NO: 2n, wherein n is an integer between 1 and 88; more preferably at least about 70% homologous to SEQ ID NO: 2n, wherein n is an integer between 1 and 88; still more preferably at least about 80% homologous to SEQ ID NO: 2n, wherein n is an integer between 1 and 88; even more preferably at least about 90% homologous to SEQ ID NO: 2n, wherein n os an initerger between 1 and 88; and most preferably at least about 95% honologous to SEQ ID NO: 2n, wherein n is an integer between 1 and 88.

An isolated nucleic acid molecule encoding a NOVX protein homologous to the protein of SEQ ID NO: 2n, wherein n is an integer between 1 and 88. can be created by introducing one or more nucleotide substitutions, additions or deletions into the nucleotide sequence of SEQ ID NO: 2n−1, wherein n is an integer between 1 and 88. such that one or more amino acid substitutions, additions or deletions are introduced into the encoded protein.

Mutations can be introduced any one of SEQ ID NO: 2n−1, wherein n is an integer between 1 and 88, by standard techniques, such as site-directed mutagenesis and PCR-mediated mutagenesis. Preferably conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, therein, tyrosine, cysteine), nonpolar side chains (e g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO: 2n−1, wherein n is an integer between 1 and 88, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g., avidin proteins).

In yet another embodiment, a mutant NOVX protein can be assayed for the ability to regulate a specific biological function (e.g.. regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NO: 2n−1, wherein n is an integer between 1 and 88, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO: 2n, wherein n is an integer between 1 and 88, or antisense nucleic acids complementary to a NOVX nucleic acid sequence of SEQ ID NO: 2n−1, wherein n is an integer between 1 and 88, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding,, strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25 30, 35. 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide can be chemically synthesized using naturally-occurring nucleotides or variously modified nucleotides designed to increase the biological stability of the molecules or to increase the physical stability of the duplex formed between the antisense and sense nucleic acids (e g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl)uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3 -amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e g., by inhibiting transcription and/or translation). The hybridization can be by conventional nucleotide complemenitarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systematic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et. al., 1987, Nucl. Acids Res. 15: 6625-6641. The antisense nucleic acid molecule can also comprise a 2′-o-methylribonucleotide (See, e.g., Inoue, et. al., 1987, Nucl. Acids Res. 15: 6131-6148) or a chimeric RNA-DNA analogue (See, e.g., Inoue, et. al., 1987, FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i e.. SEQ ID NO: 2n−1, wherein n is an integer between 1 and 88). For example, a derivative of a Tetrahymena L-19 IVS RNA can be constructed in which the nucleotide sequence of the active site is complementary to the nucleotide sequence to be cleaved in a NOVX-encoding mRNA. See, e.g, U.S. Pat. No. 4,987,071 to Cech, et. al., and U.S. Pat. No. 5,116,742 to Cech, et. al., NOVX mRNA can also be used to select a catalytic RNA having a specific ribonuclease activity from a pool of RNA molecules. See, e.g., Bartel et. al., (1993) Science 261:1411-1418.

Alternatively, NOVX gene expression can be inhibited by targeting nucleotide sequences complementary to the regulatory region of the NOVX nucleic acid (e g., the NOVX promoter and/or enhancers) to form triple helical structures that prevent transcription of the NOVX gene in target cells. See, e.g, Helene 1991. Anticancer Drug Des. 6: 569-84; Helene, et. al., 1992, Ann. N.Y. Acad. Sci. 660: 27-36;Maher, 1992, Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et. al., 1996. Biorg. Med. Chem. 4: 5-23. As used herein, the terms “peptide nucleic acids” or “PNAs” refer to nucleic acid Mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et. al., 1996, supra; Perry-O'Keefe, et. al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996. supra); or as probes or primers for DNA sequence and hybridization (See, Hyrup, et. al., 1996, supra;

Perry-O'Keefe, et. al., 1996, supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion should provide binding affinity and specificity. PNA-DNA chimeras can be linked using linkers of appropriate lengths selected in terms of base stacking number of bonds between the nucleotide bases, and orientation (see, Hyrup, et. al., 1996, supra). The synthesis of PNA-DNA chimeras can be performed as described in Hyrup, et. al., 1996. supra and Finn, et. al., 1996, Nucl. Acids Res. 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et. al., 1989, Nucl. Acid Res. 17: 5973-5988. PNA monomers are then coupled in a stepwise manner to produce a chimeric molecule with a 5′ PNA segment and a 3′ DNA segment. See, e.g., Finn, et. al., 1996, supra. Alternatively, chimeric molecules can be synthesized with a 5′ DNA segment and a 3′ PNA segment. See, e.g., Petersen, et. al., 1975. Bioorg. Med. Chem. Lett. 5: 1119-11124.

In other embodiments, the oligonucleotide may include other appended groups such as peptides (e_g., for targeting host cell receptors in vivo), or agents facilitating transport across the cell membrane (see, e.g., Letsinger, et al., 1989, Proc. Natl. Acad. Sci. U.S.A. 86: 6553-6556; Letsinger, et. al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (See, e g., Krol, et. al., 1988, BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO: 2n, wherein n is an integer between 1 and 88. The invention also includes a mutant or variant protein any of whose residues may be changed from the corresponding residues shown in any one of SEQ ID NO: 2n, wherein n is an integer between 1 and 88, while still encoding a protein that maintains its NOVX activities and physiological functions or a functional fragment thereof.

In general, a NOVX variant that preserves NOVX-like function includes any variant in which residues at a particular position in the sequence have been substituted by other amino acids, and further include the possibility of inserting, all additional residue or residues between two residues of the parent protein as well as the possibility of deleting one or mole residues from the parent sequence. Any amino acid substitution, insertion, or deletion is encompassed by the invention. In favorable circumstances,the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogenic to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombiniantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO: 2n, wherein n is an integer between 1 and 88) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO: 2n, wherein n is an integer between 1 and 88. In other embodiments, the NOVX protein is substantially homologous to SEQ ID NO: 2n, wherein n is an integer between 1 and 88, and retains the functional activity of the protein of SEQ ID NO: 2n, wherein n is an integer between 1 and 88, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO: 2n, wherein n is an integer between 1 and 88, and retains the functional activity of the NOVX proteins of SEQ ID NO: 2n, wherein n is an integer between 1 and 88.

Determining Homology Between Two or More Sequences

To determine the percent homology of two amino acid sequences or of two nucleic the sequences arc aligned for optimal comparison purposes (e.g, gaps can be introduced in the sequence of a first amino acid or nucleic acid sequence for optimal alignment with a second amino or nucleic acid sequence). The amino acid residues or nucleotides at corresponding amino acid positions or nucleotide positions arc then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J. Mol. Biol. 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, vitl the CDS (encoding) part of the DNA sequence of SLQ ID NO: 2n−1, wherein n is an integer between 1 and 88.

The term “sequence identity” refers to the degree to which two polynucleotide or polypeptide sequences are identical on a residue-by-residue basis ovei a particular region of comparison. The term “percentage of sequence identity” is calculated by comparing two optimally aligned sequences over that region of comparison, determining, the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO: 2n, wherein n is an integer between 1 and 88, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein tie NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active positions of a NOVX protein. Within the fusion protein, the term “operatively-linked” is intended to indicate that the NOVX polypeptide and the non-NOVX polypeptide are fused in-flame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobin protein family. The NOVX immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiate disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g, by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively. PCR amplification of gene fragments can be carried out using anchor primers that give rise to complementary overhangs between two consecutive gene fragments that can be subsequently be annealed and reamplified to generate a chimeric gene sequence (see, e.g., Ausubel, et. al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding, nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e g, discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Naranig, 1983. Tetrahedron39: 3; Itakura, et. al., 1984. Annu. Rev. Biochem. 53: 323; Itakura, et. al., 1984. Science 198: 1056; Ike, et. al., 1983, Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin anid Yourvan, 1992, Proc. Natl. Acad. Sci. USA 89: 7811-7815; Delgrave, et. al., 1993. Protein Engineering 6:327-331.

Anti-NOVX Antibodies

Included in the invention are antibodies to NOVX proteins, or fragments of NOVX proteins. The term “antibody” as used herein refers to immunoglobin molecules and immunologically active portions of immunoglobulin (Ig) molecules, i.e., molecules that contain an antigen binding site that specifically binds (immunoreacts with ) an antigen. Such antibodies include, but are not limited to, polyclonal, monoclonal, chimeric, single chain, F_{ab}, F_{ab}, and F_{(ab′)2} fragments and an F_{ab} expression library. In general antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO: 2n, wherein n is an integer between 1 and 88, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide arc regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Natl. Acad. Sci. USA 78: 3824-3828; Kyte and Doolittle 1982, J. Mol. Biol., 157: 105-142. each incorporated herein by reference in their entirety. Antibodies that arc specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is <1 μM, preferably <100 nM, more preferably <10 nM, and most preferably <100 pM to about 1 pM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring, Harbor, NY, incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring, immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g aluminum hydroxide), surface active substances (e.g.. lysolecithin, pluronic polyols, polyanions, peptides, oil emulsions, dinitrophenol, etc.), adjuvants usable in humans such as Bacille Calmette-Guerin and Corynebacterium parvum or similar immunostimulatory agents. Additional examples of adjuvants which can be employed include MPL-TDM adjuvant (monophosphoryl Lipid A. synthetic trehalose dicorynomycolate).

The polyclonal antibody molecules directed against the immunogenic protein can be isolated from the mammal (e g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complemenitarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or arc capable of producing antibodies that will specifically bind to the immunizing agent. Alternatively, the lymphocytes can be immunized in vitro.

The immunizing agent z ill typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes arc then fused with an immortalized cell line using a suitable fusing agents such as polyethylene glycol, to form a hybridization cell (Goding, Monoclonal Antibodies: Principles and Practice, Academic Press (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused. immortalized cells. For example, if the parental cells lack the enzyme hypoxanthine guanine phosphoribosyl transferase (HGPRT or HPRT), the culture medium for the hybridomas typically will include hypoxanthine, aminiopterin, and thymidine (“HAT medium”), which substances prevent the growth of HGPRT-deficient cells.

Preferred immortalized cell lines are those that fuse efficiently support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromycloma cell lines also have been described for the production of human monoclonal antibodies (Kozbor, J. Immunol., 133:3001 (1984); Brodeur et. al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can for example, be determined by the Scatchard analysis of Munson and Pollard, Anal. Biochem., 107:220 (1980). It is an objective especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding,1986). Suitable culture media for this purpose include, for example, Dulbecco's Modified Eagle's Medium and RPMI-1640 medium. Alternatively, the hybridoma cells call be grown in vivo as ascites in a

The monoclonal antibodies secreted by the subclones can be isolated or purified from the culture medium or ascites fluid by conventional immunoglobulin purification procedures Such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4.816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins immunoglobulin chains or fragments thereof (such as Fv, Fab, Fab′, F(ab′)₂ or other antigen-blinding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Reichmann et. al., Nature, 332:323-327 (1988); Verhoeyen et. al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin in are replaced by corresponding non-human residues. Humanized antibodies can also comprise residues which are found neither in the recipient antibody nor in the imported CDR or framework sequences. In general, the humanized antibody comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et. al., 1986; Reichmann et. al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et. al., 1983 Immunol Today 4: 72) and the EBV hybridoma technique to produce human monoclonal antibodies (see Cole, et. al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et. al., 1983. Proc. Natl. Acad. Sci. USA 80: 2026-2030) or by transforming human B-cells with Epstein Barr Virus in vitro (see Cole, et. al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et. al., J. Mol. Biol., 222:581 (1991)). Similarly, human antibodies can be made by introducing human immunoglobulin loci into transgenic animals, e.g.. mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425: 5,661 016. and in Marks et. al. (Bio/Technology, 10, 779-783 (1992)); Lonberg et. al. (Nature 368 856-859 (1994)); Morrison (Nature 368.812-13 (1994)); Fishwild et al,.(Nature Biotechnology 14, 845-51 (1996)); Neuberger (Nature Biotechnology 14, 826 (1996)): and Loneberg and Huszar (Intern. Rev. Immunol. 13 65-93 (1995)).

Human antibodies may additionally be produced using transgenic nonhuman animals which are modified so as to produce fully human antibodies rather than tile animal's endogenous antibodies in response to challenge by an antigen. (See PCT publication WO94/02602). The endogenous genes encoding, the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, tile genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including, deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding, a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et. al., 1989 Science 246: 1275-1281) to allow rapid and effective identification of monoclonal F_{ab} fragments with the desired specificity for a protein or derivatives, fragment, analogs or homologs thereof. Antibody fragments that contain the idiotypes to a protein antigen may be produced by techniques known in the art including, but not limited to: (i) an F_{(a,b′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chained/light-chain pairs where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993. and in Traunecker et. al., EMBO. J., 10:3655-3659 (1991).

Antibody variable domains with the desired binding specificities (antibody-antigen combining, sites) can be fused to immunoglobulins constant domain sequences. The fusion preferably, is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and if desired, the immunoglobulin light chain, are inserted into separate expression vectors, and are co-transfected into a suitable host organism. For further details of generating bispecific antibodies see, for example, Suresh et. al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et. al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. Tile Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et. al., J. Exp. Med. 175:217-225 (1992) describe the production of a fully humanized bispecific antibody F(ab′)₂ molecule. Each Fab′fragment was separately secreted from E. coli and subjected to directed chemical coupling, in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et. al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et. al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{II} and V_{L} domains of one fragment are forced to pair with the complementary V_{L} and V_{II}, domains of another fragment, thereby forming two antigen-blinding sites. Another strategy for making bispecific antibody fragments by the use of single-chain Fv (sFv) dimers has also been reported. See, Gruber et. al., J. Immunol. 152:5368 (1994).

Antibodies with more than two valencies are contemplated. For example, trispecific antibodies can be prepared. Tutt et. al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding, arm and an arm which binds a cytotoxic agent or a radionuclide chelator, such as EOTULBE, DPTA, DOTA, or TETA. Another bispecific antibody of interest binds the protein antigen described herein and further binds tissue factor (TF).

Heteroconjugate Antibodies

Heteroconjugate, antibodies are also within the scope of the present invention. Heteroconjugate antibodies arc composed of two covalently joined antibodies. Such antibodies have, for example, been proposed to target immune system cells to unwanted cells (U.S. Pat. No. 4.676.980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing inter-chain disulfide bond formation in this region. The homodimeric antibody thus generated can have improved internalization capability and/or increased complement-mediated cell killing and antibody-dependent cellular cytotoxicity (ADCC). See Caron et. al., J. Exp. Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et. al. Cancer Research, 53: 2560-2565 (1993). Alternatively, an antibody can be engineered that has dual Fc regions and can thereby have enhanced complement lysis and ADCC capabilities. See Stevenson et. al., Anti-Cancer Drug Design, 3: 219-230(1989).

Immunoconjugates

The invention also pertains to immunoconjugates comprising an antibody conjugated to a cytotoxic agent such as a chemotherapeutic agent, toxin (e g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain non binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restriction, phenomycin, enomycin, and the tricothecenes. A variety of radionuclides are available for the production of radioconjugated antibodies. Examples include ²¹²Bi, ¹³¹I, ¹³¹In y⁹⁰Y, and ¹⁸⁶Re.

Conjugates of the antibody and cytotoxic agent are made using a variety of bifunctional protein-coupling agents Such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et. al., Science, 238: 1098 (1987). Carbon-14-labeled 1-isothiocyanatobenzyl-3-methyldiethylene triaminiepenitaacetic acid (MX-DTPA) is an exemplary chelating agent for conjugation of radionucleotide to the antibody. See W094/11026.

In another embodiment, the antibody can be conjugated to a “receptor” (Such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et. al.. Proc. Natl. Acad. Sci. USA, 82: 3688 (1985); Hwang et. al.. Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5.01 3.556.

Particularly useful liposomes can be generated by the reverse-phase evaporation method faith a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′fragments of the antibody of the present invention can be conjugated to the liposomes as described in Martin et. al., J. Biol. Chem., 257: 286-288 (1982) via a disulfide-interchange reaction. A chemotherapeutic agent (such as Doxorubicin) is optionally contained within the liposome. See Gabizon et. al., J. National Cancer Inst., 81(19): 1484 (1989).

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Antibodies directed against a NOVX protein of the invention may be used in methods known within the art relating to the localization and/or quantitation of a NOVX protein (e.g., for use in measuring levels of the NOVX protein within appropriate physiological samples, for use in diagnostic methods, for use in imaging the protein, and the like). In a given embodiment, antibodies specific to a NOVX protein, or derivative, fragment, analog or homolog thereof, that contain the antibody derived antigen binding domain, are utilized as pharmacologically active compounds (referred to hereinafter as “Therapeutics”).

An antibody specific for a NOVX protein of the invention (e.g., a monoclonal antibody or a polyclonal antibody) can be used to isolate a NOVX polypeptide by standard techniques, such as immunoaffinity, chromatography or immunoprecipitation. An antibody to a NOVX polypeptide can facilitate the purification of a natural NOVX antigen from cells, or of a recombinantly produced NOVX antigen expressed in host cells. Moreover, such an anti-NOVX antibody can be used to detect the antigenic NOVX protein (e.g., in a cellular lysate or cell supernatant) in order to evaluate the abundance and pattern of expression of the antigenic NOVX protein. Antibodies directed against a NOVX protein can be used diagnostically to monitor protein levels in tissue as part of a clinical testing procedure, e to, for example, determine the efficacy of a given treatment regimen. Detection can be facilitated by coupling (i e.. physically linking) the antibody to a detectable substance. Examples of detectable substances include various enzymes, prosthetic groups, fluorescent materials, luminescent materials, bioluminscent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin: examples of suitable fluorescent materials include unbelliferone, fluorescein, fluorescein isothiocyanate, rhodamine, dichlorotriazinylamine fluorescein, dansyl chloride or phycoerythrin; an example of a luminescent material includes luminol; examples of bioluminscent materials include luciferase, luciferin, and aequorin, and examples of suitable radioactive material include ¹²⁵I, ¹³¹I, ³⁵S or³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases interferes with the functioning of the target, and in other cases, promotes a physiological response. The amount required to be administered will furthermore depend on the binding affinity of the antibody for its specific antigen and will also depend on the rate at which an administered antibody is depleted from the free volume other subject to which it is administered. Common ranges from therapeutically effective dosing of an antibody or antibody fragment of the invention may be, by way of nonlimiting example, from about 0.1 mg,/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et. al., editors) Mack Pub. Co., Easton, Pa.: 1995; Drug Absorption 30 Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et. al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent. cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and naniocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing( the antibody, at which matrices are in the form of shaped articles, e.g., films, or microcapsules. Examples of sustained-release matrices include polyesters, hydrogels (for example, poly(2-hydroxymethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i e., physically linking) a detectable substance to the probe or antibody, as well as indirect labeling of the probe or antibody by reactivity with another reagent that is directly labeled. Examples of indirect labelling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA protein, or genomic DNA in a biological sample in vitro as well as in vivo. For example, in vitro techniques for detection of an analyte mRNA include Northern hybridizations and in situ hybridizations. In vitro techniques for detection of all analyte protein include enzyme linked immunosorbent assays (ELISAs), Western blots, immunopecipitations, and immunofluorescence. In vitro techniques for detection of an analyte genomic DNA include Southern hybridizations. Procedures for conducting immunoassays are described. For example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e g, non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses) which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on tile basis of the host cells to be used from expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory” sequence is intended to includes promoters, enhancers and other expression control elements (e g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e g., NOVX proteins, mutant forms of NOVX proteins, fusion proteins, etc.).

The recombinant expression vectors of the invention can be designed for expression of NOVX proteins in procaryotic or eucaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein: (ii) to increase the solubility of the recombinant protein; and (iii) to aid in the purification of the recombinant protein by acting as a liganicl in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et. Al., (1988) Gene 69:301 -315) and pET 11d (Studier et. al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (.see, e.g., Wada, et. al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerevisiae include pYepSec1 (Baldari, et. al., 1987. EMBO. J. 6: 229-234), pMFa (Kurjan and Herskowitz, 1982, Cell 30: 933-943), pJRY88 (Schultz et. al., 1987, Gene 54: 113-123), pYES2 (Invitrogen Corporation, San Diego, Calif.), and picZ (In Vitrogen Corp, San Diego, Calif.).

Alternatively, NOVX can be expressed in insect cells using baculovirus expression vectors. Baculovirus vectors available for expression of proteins in cultured insect cells (e g., SF9 cells) include the pAc series (Smith, et. al., 1983, Mol. Cell Biol. 3: 2156-2165) and the pVL series (Lucklow and Summers, 1989, Virology 170: 31-39).

In yet another embodiment, a nucleic acid of the invention is expressed in mammalian cells using a mammalian expression vector. Examples of mammalian expression vectors include pCDM8 (Seed, 1987 Nature 329: 840) and pMT2PC (Kaufman, et/ al., 1987 EMBO. J. 6: 187-195). When used in mammalian cells, the expression vector's control functions are often provided by viral regulatory elements. For example, commonly used promoters are derived from polyoma, adenoviruses 2, cytomegalovirus, and simian virus 40. For other suitable expression systems for both prokaryotic and eukaryotic cells see. e.g., Chapters 16 and 17 of Sambrook, et. al., MOLECULAR CLONING: A LABORATORY MANUAL, 2nd ed., Cold Spring Harbor Laboratory Cold Spring Harbor Laboratory Press, Cold Spring Harbor, N.Y., 1989.

In another embodiment, the recombinant mammalian expression vector is capable of directing expression of the nucleic acid preferentially in a particular cell type (e g tissue-specific regulatory elements are used to express the nucleic acid). Tissue-specific regulatory elements are known in the art. Non-limiting examples of suitable tissue-specific promoters include the albumin promoter (liver-specific; Pinkert, et. al., 1987. Genes Dev. 1: 268-277), lymphoid-specific promoters (Calame and Eaton, 1988, Adv. Immunol. 43: 235-275), in particular promoters of T cell receptors (Winoto and Baltimore, 1989. EMBO. J. 8: 729-733) and immunoglobulins (Banerji, et. al., 1983, Cell 33: 729-740; Queen and Baltimore, 1983, Cell 33: 741-748), neuron-specific promoters (e.g., the neurofilament promoter; Byrne and Ruddle, 1989, Proc. Natl. Acad. Sci. USA 86: 5473-5477), pancreas-specific promoters (Edlund, et. al., 1985, Science 230: 912-916), and mammary gland-specific promoters (e.g., milk whey promoter; U.S. Pat. No. 4,873,316 and European Application Publication No. 264,166). Developmentally-regulated promoters are also encompassed, e.g, the murine box promoters (Kessel and Gruss, 1990, Science 249: 374-379) and the α-fetoprotein promoter (Campes and Tilghman, 1989, Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et.al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used inter-changeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included Within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et. al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418. hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-limiting transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i e., any one of SEQ ID NO: 2n−1, wherein n is an integer between 1 and 88, can be introduced as a transgene into the genome of a non-human animal. Alternatively, a non-human homologue of the human NOVX gene, such as a mouse NOVX genie, can be isolated based on hybridization to the human NOVX cDNA (described further supra) and used as a transgene. Intronic sequences and polyadenylation signals can also be included in the transgene to increase the efficiency or expression of the transgenic. A tissue-specific regulatory sequence(S) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. PAT. Nos. 4,736,866: 4,870,009; and 4,873,191; and Hogan, 1986. In: MANIPULATING the MOUSE EMBRYO, Cold Spring Harbor Laboratory Press, Cold Spring Harbor. N.Y. Similar methods are used for production of other transgenic animals. A transgenic founder animal can be identified based upon the presence of the NOVX transgenic in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying transgenes.

To create a homologous recombinant animal, a vector is prepared which contains at least a portion of a NOVX gene into which a deletion, addition or substitution has been introduced to thereby alter, e.g, functionally disrupt, the NOVX gene. The NOVX gene can be a human gene (e.g., the cDNA of any one of SEQ ID NO: 2n−1, wherein n is an integer between 1 and 88), but more preferably, is a non-human homologue of a human NOVX gene. For example, a mouse homologue of human NOVX gene of SEQ ID NO: 2n−1, wherein n is an integer between 1 and 88, can be used to construct a homologous recombination vector suitable for altering an endogenous NOVX gene in the mouse genome. In one embodiment, vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX genie is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas et. al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, eg., Li, et. al., 1992, Cell 69: 915.

The selected cells are then injected into a blastocyst of an animal (e.g., a mouse) to form aggregation chimeras. See, e.g. Bradley, 1987. In: TERATORCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991, Curr. Opin. Biotechnol. 2: 823-829; PCT International Publication Nos.: WO 90/1 1354; WO 91/01140; WO 92/0968; and WO 93/04169.

In another embodiment, transgenic non-humans animals can be produced that contain selected systems that allow for regulated expression of the transgene. One example of such a system is the cre/loxP recombining system of bacteriophage P1. For a description of the cre/loxP recombining system, See, e.g., Lakso, et. al., 1992, Proc. Natl. Acad. Sci. USA 89: 6232-6236. Another example of a recombinase system is the FLP recombinase system of Saccharomyces cerevisiae. See, O'Gorman, et. al., 1991, Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according-J to the methods described in Wilmutt, et. al., 1997, Nature 385: 810-813. In brief, a cell (e.g., a somatic cell) from the transgenic animal can be isolated and induced to exit the (growth cycle and enter G₀ phase. The quiescent cell can then be fused, e.g., through the use of electrical pulses, to an enucleated oocyte from an animal of the same species from Which the quiescent cell is isolated. The reconstructed oocyte is then cultured such that it develops to morula or blastocyte and then transferred to pseudopregnant female foster animal. The offspring borne of this female foster animal will be a clone of the animal from Which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of Such carriers or diluents include, but are not limited to, water, saline, finger's solutions, dextrose solution, and ⁵% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents Such as ethylenediaminetetraacetic acid (EDA); buffers such as acetates, citrates or phosphates, and agents for the ad adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor El ∩ (BASF. Parsippany,. N.J.) or phosphate buffered saline (PBS). In all cases, the composition must be sterile and should be fluid to the extent that easy syringeability exists. It must be stable under the conditions of manufacture and storage and must be preserved against the contaminating action of micro such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g, a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin an excipient such as starch or lactose, a disintegrating agent such as alginic acid, Primogel, or corn starch: a lubricant such as magnesium stearate or Sterotes, a glidant such as colloidal silicon dioxide; a sweetening agent such as sucrose or saccharin; or a flavoring agent such as peppermint, methyl salicylate, or orange flavoring.

For administration by inhalation., the compounds are delivered in the form of an aerosol spray from pressured container or dispenser which contains a suitable propellant. e.g., a gas as such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g with conventional suppository bases Such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing, a predetermined quantity of active compound calculated to produce the desired therapeutic effect in association with the required pharmaceutical carrier. The specification for the dosage unit forms of the invention are dictated by and directly dependent on the unique characteristics of tie active compound and the particular therapeutic effect to be achieved, and the limitations inherent in the art of compounding Such an active compound for the treatment of individuals.

The nucleic acid molecules of the invention can be inserted into vectors and used as gene therapy vectors. Gene therapy vectors can be delivered to a subject by, for example, intravenous injection, local administration (See, e g., U.S. Pat. No. 5,328,470) or by stereotatic injection (see, e.g., Chen, et al., 1994, Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screeching) assays described herein and uses thereof for treatments as described, supra.

Screening Assays

The invention provides a method (also referred to herein as a “screening assay”) for identifying, modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997, Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et. al., 1993, Proc. Natl. Acad. Sci, U.S.A. 90: 6909; Erb, et. al., 1994, Proc. Natl. Acad. Sci. USA 91: 11422; Zuckermann, et. al., 1994, J. Med. Chem. 37: 2678; Cho, et. al., 1993. Science 261: 1303; Carrell, et. al., 1994, Angew. Chem. Ed. Engl. 33:

2059; Carell, et al., 1994, Angew Chem. Ed. Engl. 33: 2061; and Gallop, et. al., 1994, J. Med. Chem. 37: 1233.

Libraries of compounds may be presented in solution (e.g., Houghten, 1992, Biotechniques 13: 412-421), or on beads (Lam. 1991, Nature 354: 82-84), on chips (Foder, 1993, Nature 364: 555-556), bacteria (Ladner, U.S. Pat. No. 53,22,409), spores (Ladner, U.S. Pat. No. 5,233,409), plasmids (Cull, et. al., 1992, Proc. Natl. Acad. Sci. USA 89: 1865-1869) or on phage (Scott and Smith, 1990, Science 249: 386-390; Devlin, 1990, Science 20 249: 404-406; Cwirla, et. al., 1990, Proc. Natl. Acad. Sci. USA. 87: 6378-6382; Felici, 1991. J. Mol. Biol. 222: 301-310; Ladner, U.S. Pat. No. 5,233,409.).

In one embodiment, an assay is a cell-based assay in herein, a cell which expresses a membrane-bound form of NOVX protein, or a biologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof call be accomplished, for example, by determining the ability of the NOVX protein to bind to or interact with a NOVX target molecule. As used herein, a “target molecule” is a molecule with which a NOVX protein binds or interacts in nature, for example a molecule on the surface of a cell which expresses a NOVX interacting protein a molecule on the Surface of a second cell, a molecule in the extracellular milieu, a molecule associated with the internal surface of a cell membrane or a cytoplasmic molecule. A NOVX target molecule call be a non-NOVX molecule or a NOVX protein or polypeptide of the invention. In one embodiment, a NOVX target molecule is a component of a signal transduction pathway that facilitates transduction of an extracellular signal (e.g., a signal generated by binding of a compound to a membrane-bound NOVX molecule) through,h the cell membrane and into the cell. The target, for example, call be a second intercellular protein that has catalytic activity or a protein that facilitates the association of downstream signaling, molecules With NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one Such embodiment, the assay comprises contacting the NOVX protein or biologically-active portion thereof with a known compound which binds NOVX to form an assay mixture contacting the assay mixture with a test compound, and determining, the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining( the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct biding in an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate Substrate call be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114,Thesit®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter- plates, test tubes, and micro-centrifuge tubes. In one embodiment, a fusion protein can be provided that adds a domain that allows one or both of the proteins to be bound to a matrix. For example. GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein and the mixture is incubated under conditions conducive to complex formation (e.,g., at physiological conditions for salt and pH). Following incubation the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening, assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e. statistically significantly greater) in the presence of the candidate compound than in its absence, the candidate compound is identified as a stimulator of NOVX m1RNA or protein expression. Alternatively, when expression of NOVX mRNA or protein is less (statistically significantly less) in the presence of the candidate compound than in its absence, the candidate compound is identified as an inhibitor of NOVX mRNA or protein expression.

The level of NOVX mRNA or protein expression in the cells can be determined by methods described herein for detecting( NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et. al., 1993, Cell 72: 223-232; Madura, et. al., 1993, J. Biol. Chem. 268: 12046-12054; Bartel, et. al., 1993, Biotechniques 14: 920-924; Iwabuchi, et. at., 1993, Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual loom a minute biological sample (tissue typing,); and (iii) aid in forensic identification of a biological sample. Some of these applications are described in the subsections, below.

Chromosome Mapping

Once the sequence (or a portion of the sequences) of a gene has been isolated, this sequence can be used to map the location of the gene on a chromosome. This process is called chromosome mapping. Accordingly, portions or fragments of the NOVX sequences of SEQ ID NO: 2n−1, wherein, is an integer between 1 and 88, or fragments or derivatives thereof, can be used to map the location of the NOVX genes, respectively, on a chromosome. The mapping of the NOVX sequences to chromosomes is an important first step in correlating these sequences with (genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et. al., 1983, Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycle. Using, the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using, cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, ill suffice to get good results at a reasonable amount of time. For a review of this technique, see, Verma, et. al. HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et. al., 1987, Nature 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutilations from polymorphisms.

Tissue Typing

The NOVX sequences or the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (restriction ligament length polymorphisms, described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation Occurs to some degree in the coding regions of these sequences, and to a greater degree in the noncoding regions. It is estimated that allelic variation between individual humans occurs with a frequency of about once per each 500 bases. Much of the allelic variation is due to shingle nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO: 2n−1, wherein n i s an integer between 1 and 88, are used, a more appropriate number of primers for positive individual identification would be 500-2,000.

Predictive Medicine

The invention also pertains to the field of predictive medicine in which diagnostic assays, prognostic assays, pharmacogenomic, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine %whether an individual is afflicted wvitlh a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders Alzheimer's Disease, Parkinison's Disorder, immune disorders, and hemlatopoietic disorders, and the various dyslipidemias, metabolic disturbances associated with obesity, the metabolic syndrome X and wasting disorders associated With chronic diseases and various cancers. The invention also provides for prognostic (or predictive) assays for determining( whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein Such that the presence of NOVX is detected in the biological sample. An agent for detecting NOVX mRNA or genomic DNA is a labeled nucleic acid probe capable hybridizing to NOVX mRNA or genomic DNA. The nucleic acid probe can be, for example, a full-length NOVX nucleic acid, such as the nucleic acid of SEQ ID NO: 2n−1, wherein n is an integer between 1 and 89 or portion thereof, such as an oligonucleotide of at least 15. 30, 50. 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody Faith a detectable label. Antibodies can be polyclonal, or mire preferably, monoclonal. An intact antibody, or a flagment thereof (e.g., Fab or F(ab′)₂) can be used. The term “labeled”. With regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vito techniques for detection of NOVX mRNA include Northern hybridizations and in situ hybridizations. In vitro techniques for detection of NOVX protein include enzyme linked immunosorbent assays (ELISAs), Western blots, immunopecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood Leukocyte sample isolated by conventional means from a subject.

In another embodiment, the methods further involve obtaining a control biological sample from a control subject, contacting the control sample with a Compound or agent capable of detecting NOVX protein, mRNA, or genomic DNA, such that the presence of NOVX protein, mRNA or genomic DNA is detected in the biological sample, and comparing the presence of NOVX protein. mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using, the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e g., an agonist, antagonist, peptidomimetics, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated smith an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesions gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX genie. For example. Such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see. e.g., Landegran, et. al., 1988, Science 241: 1077-1080; and Nakazawa, et. al., 1994, Proc. Natl. Acad. Sci. USA 91: 360-364), the latter of which can be particularly useful for detecting point mutations in the NOVX-gene (see, Abravaya, et. al., 1995, Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see. Guatelli, et. al., 1990, Proc. Natl. Acad. Sci. USA 87 1874-1 878) transcriptional amplification system (see, Kwoh, et. al., 1989, Proc. Natl. Acad. Sci. USA 86:11 73-1177); Qβ Replicase (see, Lizardi, et. al., 1988, BioTechnology 6: 11 97), or any other nucleic acid amplification method, followed by the detection of the amplified molecules using techniques well known to those of skill in the art. These detection schemes are especially useful for the detection of nucleic acid molecules if such molecules are present in very lo%, numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et. al., 1996, Human Mutation 7: 244-255; Kozal, et. al., 1996, Nat. Med. 2: 753-759. For example, genetic mutations in NOVX can be identified in two dimensional arrays containing light-generated DNA probes as described in Cronin, et. al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets one complementary to the wild-type gene and the other complementary to the mutant gene.

In yet another embodiment, any of a variety of sequencing( reactions known in the art can be used to directly sequence the NOVX gene and detect mutations by comparing the sequence of the sample NOVX with the corresponding wild-type (control) sequence. Examples of sequencing, reactions include those based on techniques developed by Maxim and Gilbert, 1977, Proc. Natl. Acad. Sci. USA 4 74: 560 or Sanger, 1977, Proc. Natl. Acad. Sci. USA, 74: 5463. It is also contemplated that any of a variety of automated sequencing procedures can be utilized when performing the diagnostic assails (see, e (e Naeve, et. al., 1995, Biotechiques 19: 448), including sequencing by mass spectrometry (see. e.g., PCT International Publication No. WO 94/16101; Cohen, et. al., 1996 Adv. Chromatography 36: 127-162; and Griffin, et. al., 1993, Appl. Biochem Biotechnol. 38: 147-159).

Other methods for detecting mutations in the NOVX genie include methods in which protection from cleavage agents is used to detect mismatched bases in RNA/RNA or RNA/DNA heteroduplexes. See, e.g., Myers, et. al., 1985, Sciences 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et. al., 1988, Proc. Natl. Acad. Sci. USA 85: 4397; Saleeba, et. al., 1992, Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et. al., 1994, Cacincogenesis 15: 1657-1662. According to an exemplary embodiment, a probe based on a NOVX sequence, e g. a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,.459,039.

In other embodiments, alterations in electrophoretic mobility will be used to identify mutations in NOVX genes. For example, single strand conformation polymorphisms (SSCP) may be used to detect differences in electrophoretic mobility between mutant and wild type nucleic acids. See, e.g , Orita, et. al., 1989, Proc. Natl. Acad. Sci. USA: 86: 2766; Cotton, 1993, Mutat. Res. 285: 125-144; Hayashi, 1992, Genet. Anal. Tech. Appl. 9: 73-79. Single-stranded DNA fragments of sample and control NOVX nucleic acids ill be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is mole Sensitive to a change in sequence. In one embodiment, the subject method utilizes heteroduplex analysis to separate double stranded heteroduplex molecules on the basis of changes in electrophoretic mobility. See, e.g. Keen, et. al., 1991, Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et. al., 1985, Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987, Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et. al., 1986, Nature 324: 163; Saiki, et. al., 1989, Proc. Natl. Acac. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g. Gibbs. et. al., 1989, Nucl. Acids Res. 17: 2437-2448) or at the extreme 3′-terminus of one primer where, under appropriate conditions, mismatch can prevent, or reduce polymerase extension (see, e.g., Prossner, 1993, Tibtech. 1: 238). In addition it may be desirable to introduce a novel restriction site in the region of the mutation to create cleavage-based detection. See, e.g., Gasparini, et. al., 1992, Mol. Cell Probes 6: 1. It is anticipated that in certain embodiments amplification may also be performed using, Taq ligase or amplification. See e.g., Barany, 1991, Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amidification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders. The disorders include but are not limited to, e.g.. those diseases, disorders and conditions listed above, and more particularly include those diseases, disorders, or conditions associated with homologs of a NOVX protein, such as those summarized in Table A.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering( the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacologic deals with clinically significant hereditary variations in the response to drugs Clue to altered drug disposition and abnormal action in affected persons. See e.g., Eichelbaum, 1996, Clin. Exp. Pharmacol. Physiol., 23: 983-985; Linder, 1997, Clin. Chem., 43: 254-266. In general, two types of pharmacogenetic conditions call be differentiated. Genetic conditions transmitted as a single factor altering, the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome pregnancy zone protein precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX proteins expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g, drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or unregulated NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including, NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agents (ii) detecting the level of expression of a NOVX protein, mRNA, or genomic DNA in the preadministration sample; (iii) obtaining one or more post-administration samples from the subject; (iii) detecting the level of expression or activity of the NOVX protein. mRNA, or genomic DNA i tile post-administration samples; (v) comparing the level of expression or activity of the NOVX protein, mRNA, or genomic DNA in the pre-administration sample with the NOVX protein, mRNA, or genomic DNA i n the post ad ministration sample or samples; and (ii) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include but are not limited to, e.g., those diseases, disorders and conditions listed above, and more particularly include those diseases, disorders, or conditions associated with homologs of a NOVX protein, such as those summarized in Table A.

These methods of treatment will be discussed more fully, below.

Diseases and Disorders

Diseases and disorders that are characterized by increased (relative to a subject not suffering from the disease or disorder) levels or biological activity may be treated with Therapeutics that antagonize (i.e.. reduce or inhibit) activity. Therapeutics that antagonize activity may be administered in a therapeutic or prophylactic manner. Therapeutics that may be utilized include, but are not limited to: (i) an aforementioned peptide or analogs, derivatives, fragments or homologs thereof; (ii) antibodies to an aforementioned peptide; (iii) nucleic acids encoding an aforementioned peptide; (ill) administration of antisense nucleic acid and nucleic acids that are -dysfunctional (i.e., due to a heterologous insertion within the coding sequences of coding sequences to an aforementioned peptide) that are utilized to “knockout” endogenous function of all aforementioned peptide by homologous recombination (see, e.g. Capecchi, 1989, Science 244: 1288-1 292); or (v) modulators (i.e., inhibitors, agonist and antagonists, including additional peptide mimetic of the invention or antibodies specific to a peptide of the invention) that alter the interaction between an aforementioned peptide and its binding partner.

Diseases and disorders that are characterized by decreased (relative to a subject not suffer-in(g from the disease or disorder) levels or biological activity may be treated with Therapeutics that increase (i.e., are agonists to) activity. Therapeutics that unregulated activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vivo for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in sit i hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Method

Another aspect of the invention pertains to methods of modulation, NOVX expression or activity for therapeutic purposes. The modulatory method of the invention involves contacting a cell with an agent that modulates one or more of the activities of NOVX protein activity associated With the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of Such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vito (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein, or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice chicken, cows, monkeys, rabbits, and the like, prior to testing will human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders. The disorders include but are not limited to, e.g., those diseases, disorders and conditions listed above, and more particularly include those diseases, disorders, or conditions associated with homologs of a NOVX protein, such as those summarized in Table A.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from diseases, disorders, conditions and the like, including but not limited to those listed herein.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A

Polynucleotide and Polypeptide Sequences, and Homology Data

The NOV1 clone was analyzed, and the nucleotide and encoded polypeptide sequences are shown in Table 1A.

TABLE 1A
NOV1 Sequence Analysis

| | SEQ ID NO:1 | 975 bp |
|---|---|---|
| NOV1a, | GTCCTTGGAGGCCAGAGGGGACTCTGAGCATCGGAAAGCAGGATGCCTGGTTTGCTTT | |
| CG102071-03 | TATGTGAACCGACAGAGCTTTACAACATCCTGAATCAGGCCACAAAACTCTCCAGATT | |
| DNA | AACAGACCCCAACTATCTCTGTTTATTGGATGTCCGTTCCAAATGGGAGTATGACGAA | |
| Sequence | AGCCATGTGATCACTGCCCTTCGAGTGAAGAAGAAAAATAATGAATATCTTCTCCCGG | |
| | AGTCTGTGGACCTGGAGTGTGTGAAGTACTGCGTGGTGTATGATAACAACAGCAGCAC | |
| | CCTGGAGATACTCTTAAAAGATGATGATGATGATTCAGACTCTGATGGTGATGGCAAA | |
| | GATCTTGTGCCTCAAGCAGCCATTGAGTATGGCAGGATCCTGACCCGCCTCACCCACC | |
| | ACCCCGTCTACATCCTGAAAGGGGGCTATGAGCGCTTCTCAGGCACGTACCACTTTCT | |
| | CCGGACCCAGAAGATCATCTGGATGCCTCAGGAACTGGATGCATTTCAGCCATACCCC | |
| | ATTGAAATCGTGCCAGGGAAGGTCTTCGTTGGCAATTTCAGTCAAGCCTGTGACCCCA | |
| | AGATTCAGAAGGACTTGAAAATCAAAGCCCATGTCAATGTCTCCATGGATACAGGGCC | |
| | CTTTTTTGCAGGCGATGCTGACAAGCTTCTGCACATCCGGATAGAAGATTCCCCGGAA | |
| | GCCCAGATTCTTCCCTTCTTACGCCACATGTGTCACTTCATTGGGTATCAGCCGCAGT | |
| | TGTGCCGCCATCATAGCCTACCTCATGCATAGTAACGAGCAGACCTTGCAGAGGTCCT | |
| | GGGCCTATGTCAAGAAGTGCAAAAACAACATGTGTCCAAATCGGGGATTGGTGAGCCA | |
| | GCTGCTGGAATGGGAGAAGACTATCCTTGGAGATTCCATCACAAACATCATGGATCCG | |
| | CTCTACTGATCTTCTCCGAGGCCCACCGAAGGGTACTGAAGAGCCTC | |
| | ORF Start: ATG at 43 | ORF Stop: TAG at 784 |
| | SEQ ID NO:2 | 247 aa MW at 28330.1 kD |

| | |
|---|---|
| NOV1a | MPGLLLCEPTELYNILNQATKLSRLTDPNYLCLLDVRSKWEYDESHVTTALRVKKKNN |
| CG102071-03 | EYLLPESVDLECVKYCVVYDNNSSTLETLLKDDDDDSDSDGDGKDLVPQAAIEYGRIL |
| Protein | TRLTHHPVYILKGGYERFSGTYHFLRTQKIIWMPQELDAPQPYPTEIVPGKVFVGNFS |
| Sequence | QACDPKTQKDLKIKAHVNVSMDTGRPFAGDADKLLHIRTEDSPEAQILPFLRHMCHFI |
| | GYQPQLCRHHSLPHA |

Further analysis of the NOV1 a protein yielded the following properties shown in Table 1B.

TABLE 1B
Protein Sequence Properties NOV1a

| | |
|---|---|
| PSort | 0.4500 probability located in cytoplasm; 0.3000 probability |
| analysis: | located in microbody (peroxisome); 0.1000 probability located |
| | in mitochondrial matrix space; 0.1000 probability located in |
| | lysosome (lumen) |
| SignalP | No Known Signal Sequence Predicted |
| analysis: | |

A search of the NOV1 a protein against the Geneseq database, a proprietary database that contains sequences published in patents and patent publication, yielded several homologous proteins shown in Table 1C.

TABLE 1C
Geneseq Results for NOV1a

| | | NOV1a | | |
|---|---|---|---|---|
| | Protein/Organism/ | Residues/ | Identities/ | |
| Geneseq | Length [Patent #, | Match | Similarities for the | Expect |
| Identifier | Date] | Residues | Matched Region | Value |
| AAY44241 | Human cell signalling protein-4- | 1 . . . 232 | 231/232 (99%) | e−137 |
| | Homo sapiens, 313 aa. [WO9958558- | 1 . . . 232 | 232/232 (99%) | |
| | A2, 18 Nov. 1999] | | | |
| AAY07958 | Human secreted protein fragment #2 | 71 . . . 232 | 162/162 (100%) | 1e−93 |
| | encoded from gene 6-Homo sapiens, | 34 . . . 195 | 162/162 (100%) | |
| | 276 aa. [WO9918208-A1, | | | |
| | 15 Apr. 1999] | | | |
| AAM91270 | Human immune/haematopoietic | 151 . . . 209 | 56/59 (94%) | 1e−26 |
| | antigen SEQ ID NO: 18863-Homo | 61 . . . 119 | 57/59 (95%) | |
| | sapiens, 123 aa. [WO200157182-A2, | | | |
| | 9 Aug. 2001] | | | |
| AAG01344 | Human secreted protein, SEQ ID NO: | 1 . . . 59 | 55/59 (93%) | 7e−26 |
| | 5425-Homo sapiens, 125 aa. | 1 . . . 59 | 57/59 (96%) | |
| | [EP1033401-A2, 6 Sep. 2000] | | | |
| ABB68968 | Drosophila melanogaster polypeptide | 160 . . . 215 | 23/57 (40%) | 0.005 |
| | SEQ ID NO 33696-Drosophila | 89 . . . 145 | 32/57 (55%) | |
| | melanogaster, 348 aa. | | | |
| | [WO200171042-A2, 27 Sep. 2001] | | | |

In a BLAST search of public sequence databases, the NOV1a protein was found to have homology to the proteins shown in the BLASTP data in Table 1D.

TABLE 1D
Public BLASTP Results for NOV1a

| | | NOV1a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9Y6J8 | Map kinase phosphatase-like protein | 1 . . . 232 | 232/232 (100%) | e−137 |
| | MK-STYX-Homo sapiens (Human), | 1 . . . 232 | 232/232 (100%) | |
| | 313 aa. | | | |
| Q9UK07 | Map kinase phosphatase-like protein | 46 . . . 232 | 187/187 (100%) | e−108 |
| | MK-STYX-Homo sapiens (Human), | 1 . . . 187 | 187/187 (100%) | |
| | 221 aa (fragment). | | | |
| Q9DAR2 | Adult male testis cDNA, RIKEN full- | 1 . . . 232 | 153/240 (63%) | 5e−92 |
| | length enriched library, | 1 . . . 240 | 200/240 (82%) | |
| | clone: 1700001J05, full insert | | | |
| | sequence-Mus musculus (Mouse), | | | |
| | 321 aa. | | | |
| Q9UKG3 | Alternatively spliced dual specificity | 149 . . . 247 | 99/99 (100%) | 8e−55 |
| | phosphatase inhibitor MK-STYX- | 1 . . . 99 | 99/99 (100%) | |
| | Homo sapiens (Human), 99 aa | | | |
| | (fragment). | | | |
| Q9UKG2 | Alternatively spliced dual specificity | 149 . . . 232 | 84/84 (100%) | 4e−44 |
| | phosphatase inhibitor MK-STYX- | 1 . . . 84 | 84/84 (100%) | |
| | Homo sapiens (Human), 101 aa | | | |
| | (fragment). | | | |

PFam analysis predicts that the NOV1 a protein contains the domains shown in the Table 1E.

TABLE 1E
Domain Analysis of NOV1a

| | | Identities/ | |
|---|---|---|---|
| Pfam | | Similarities for | Expect |
| Domain | NOV1a Match Region | the Matched Region | Value |
| Rhodanese | 18 . . . 137 | 31/155 (20%) | 0.0041 |
| | | 86/155 (55%) | |

### Example 2

The NOV2 clone was analyzed, and the nucleotide and encoded polypeptide sequences are shown in Table 2A.

TABLE 2A
NOV2 Sequence Analysis

| | SEQ ID NO:3 | 1369 bp |
|---|---|---|
| NOV2a, | CACCGAGACGCGCCGGCGGACCGCGGGCGAGTGCAGCCGGTGACCCGGCGAGAGGCGG | |
| CG102734-01 | CGCCGCTCCCAAGATGTCGCAGACGGCCATGTCCGAAACCTACGATTTTTTGTTTAAG | |
| DNA | TTCTTGGTTATTGGAAATGCAGGAACTGGCAAATCTTGCTTACTTCATCAGTTTATTG | |
| Sequence | AAAAAAAATTCAAAGATGACTCAAATCATACAATAGGAGTGGAATTTGGTTCAAAGAT | |
| | ATAAATGTTGGTGGTAAATATGTAGTTACATATGGGATACAGCAGGACAAGAA | |
| | CGATTCAGGTCCGTGACGAGAAGTTATTACCGAGGCGCGGCCGGGGCTCTCCTCGTCT | |
| | ATGATATCACCAGCCGAGAAAAACCTACAATGCGCTTACTAATTGGTTAACAGATGCCCG | |
| | AATGCTAGCGAGCCAGAACATTGTGATCATCCTTTGTGGAAACAAGAAGGACCTGGAT | |
| | GCAGATCGTGAAGTTACCTTCTTAGAAGCCTCCAGATTTGCTCAAGAAAATGAGCTGA | |
| | TGTTTTTGGAAACAAGTGCGCTCACAGGGGAGAATGTAGAAGAGGCTTTTGTACAGTG | |
| | TGCAAGAAAAATACTTAACAAATCGAATCAGGTGAGCTGGACCCAGAAAGAATGGGC | |
| | TCAGGTATTCAGTACGGAGATGCTGCCTTGAGACAGCTGAGGTCACCGCGGCGCGCAC | |
| | AGGCCCCGAACGCTCAGGAGTGTGGTTGTTAGGAGAGCACACAGGTGTTCATACAGTG | |
| | GCATTTGGGACACAATCGTTGGAACCTGAAGAATCTGAAGTTTTTTTTACCACCATCT | |
| | TTTTCTACTCTGTATGGAAGTAGATCTTTATGGGGAAAAGAGAATTTGGGGTGTTCTG | |
| | CAAGCCAGTCAAAGTGGCACAGCAAATCATATAAATCGAATTAAATGGACAACACCGT | |
| | TAGATGTGTATGTAAAAATTTTCTGTTTCATATTTTTCCTTTCACTTTCGGTTTAAAA | |
| | CATGCTATATGTACTGTATGTCCTGTAGCCCAGTGCGGCTCCACAGCATGGAATCTGA | |
| | TGTATGATATGATAGAATGTGGCACTAAATGCAGTTTCAGATTTTATTTTTTTTAATC | |
| | ATATGAACTAAAATTGTCAATTGTGAGGTGTGCTTTTCTCATCATGTTGGTTATATTG | |
| | CACAATTGGTTATATTTATGACCTGATATTCAAAGACTCTGGCATTGATAGCCAGTGT | |
| | GTTTTCTTATTTAACTCCGTTTACTACATTCTACATGGTGTTTACGTGATCCACACTT | |
| | GAAATACTAGATCAGTAGACATTCACTAATATACCAAAATAAAATGAAAAATTGAGTT | |
| | TTTCCGTGAAAAAAAAAAAAAAAAAAAAAAAAAAA | |
| | ORF Start: ATG at 72 | ORF Stop: TAG at 726 |
| | SEQ ID NO:4 | 218 aa MW at 24389.4 kD |
| NOV2a, | MSQTAMSETYDFLPKFLVIGNAGTGKSCLLHQFIEKKFKDDSNHTIGVEFGSKIINVG | |
| CG102734-01 | GKYVKLQIWDTAGQERFFRSVTRSYYRGAAGALLVYDITSRETYNALTNWLTDARMLAS | |
| Protein | QNIVIILCGNKKDLDADREVTFLEASRFAQENELMFLETSALTGENVEEAFVQCARKI | |
| Sequence | LNKTESGELDPERMGSGIQYGDAALRQLRSPRRAQAPNAQECGC | |
| | SEQ ID NO:5 | 1747 bp |
| NOV2b, | GCCGGACGGAGGGTGGAGGGCCCTGCGCCTGCGCGGAGCTGGAGTCCGGCTGGGCCGC | |
| CG102734-02 | AGCCGCTGGGAGACCGGCGGTTGCCGTGGGGACCGGTCGGGCCCCTCCCTCCTCCGGT | |
| DNA | CCCCCGCCCCAGGTCCTTCCCCACCGAGACGCGCCGGCGGACCGCGGGCGAGTGCAGC | |
| Sequence | CGGTGACCCGGCGAGAGGCGGCGCCGCTCCCAAGATGTCGCAGACGGCCATGTCCGAA | |
| | ACCTACGATTTTTTGTTTAGTTCTTGGTTATTGGAATGCAGGAACTGGCAATCTT | |
| | GCTTACTTCATCAGTTTATTGAAAAAAAAATGTCCGTGACGAGAAGTTATTACCGAGG | |
| | CGCGGCCGGGGCTCTCCTCGTCTATGATATCACCAGCCGAGAAACCTACAATGCGCTT | |
| | ACTAATTGGTTAACAGATGCCCGAATGCTAGCGAGCCAGAACATTGTGATCATCCTTT | |
| | GTGGAACAAGAAGGACCTGGATGCAGATCGTGAAGTTACCTTCTTAGAAGCCTCCAG | |
| | ATTTGCTCAAGAATGAGCTGATGTTTTTGGAAACAAGTGCGCTCACAGGGGAGAAT | |
| | GTAGAAGAGGCTTTTGTACAGTGTGCAAGAAAAATACTTAACAAAATCGAATCAGGTG | |
| | AGCTGGACCCAGAAAGAATGGGCTCAGGTATTCAGTACGGAGATGCTGCCTTGAGACA | |
| | GCTGAGGTCACCGCGGCGCGCACAGGCCCCGAACGCTCAGGAGTGTGGTTGTTAGGAG | |
| | AGCACACAGGTGTTCATACAGTGGCATTTGGGACACAATCGTTGGAACCTGAAGAATC | |
| | TGAAGTTTTTTTTACCACCATCTTTTTCTACTCTGTATGGAAGTAGATCTTTATGGGG | |
| | AAGAGAATTTGGGGTGTTCTGCAAGCCAGTCAAAGTGGCACAGCAAATCATATAAA | |
| | TCGAATTAAATGGACAACACCGTTAGATGTGTATGTAAAAAAAATTTTCTGTTTCATATTT | |
| | TTCCTTTCACTTTCGGTTTAAAACATGCTATATGTACTGTATGTCCTGTAGCCCAGTG | |
| | CGGCTCCACAGCATGGAATCTGATGTATGATATGATAGAATGTGGCACTAAATGCAGT | |
| | TTCAGATTTTATTTTTTTTAATCATATGAACTAATTGTCAATTGTGAGGTGTGCTT | |
| | TTCTCATCATGTTGGTTATATTGCACAATTGGTTATATTTATGACCTGATATTCAAAG | |
| | ACTCTGGCATTGATAGCCAGTGTGTTTTCTTATTTAAAACTCCGTTTACTACATTCTACA | |
| | TGGTGTTTACGTGATCCACACTTGAATACTAGATCAGTAGACATTCACTAATATACC | |
| | AAAATAAAATGAAAAATTGAGTTTTTCCGTGAACTTTATACTGTCCAGCTCTGTTGAT | |
| | TTTAAAGCCTCTTCATCCAGGTCAGTTCAGGIAAGTATATCTGGAGTACCTGCTCTGTT | |
| | TTTGGCTGTGAGACTAGCACTAAGGATTCTGGTACCTTTACCCAAACCTACTGGGCTA | |
| | CTAATACTTCTCTCAGCAGTTGATCAAAAATACAATAGACCATGTAAGCTGGGGCCGCTC | |
| | ATCCACTTCCAGTTTGCTGGTCTCCCTGCTAGAAAAAACACATTGTACTGTGCTTTTTCT | |
| | GGAATTCAGTATAATGGCATCACTGCCTGTTTTTCACATCTTTTGTTTCCTGTTCATT | |
| | TTAAGGAAACCTACTAAATCCAGTTAATATTAAATGGACACCACTCAAAAAAAAAAAA | |
| | ORF Start: ATG at 209 | ORF Stop: TAG at 749 |
| | SEQ ID NO:6 | 180 aa MW at 20083.6 kD |
| NOV2b, | MSQTANSETYDFLFKFLVTGNAGTGKSCLLHQFIEKKMSVTRSYYRGAAGALLVYDIT | |
| CG102734-02 | SRETYNALTNWLTDARMLASQNIVITLCGNKKDLDADREVTFLEASRFAQENELMFLEI | |
| Protein | TSALTGENVEEAFVQCARKILNKTESGELDPERMGSGIQYGDAAALRQLRSPRRAQAPN | |
| Sequence | AQECGC | |
| | SEQ ID NO:7 | 687 bp |
| NOV2c, | CGCGGATCCACCATGTCGCAGACGGCCATGTCCGIAAAAlAACCTACGATTTTTTGTTTAAGT | |
| 209829447 | TCTTGGTTATTGGAAATGCAGGAACTGGCAAATCTTGCTTACTTCATCAGTTTATTGA | |
| DNA | AAAAAAAATTCAAAGATGACTCAAAATCATACAATAGGAGTGGAATTTGGTTCAAGATA | |
| Sequence | ATAAATGTTGGTGGTAAATATGTAAAGTTACTAAATGGGATACAGCAGGACAAGAAC | |
| | GATTCAGGTCCGTGACGAGAAGTTATTACCGAGGCGCGGCCGGGGCTCTCCTCGTCTA | |
| | TGATATCACCAGCCGAGAACCTACAATGCGCTTACTAATTGGTTAACAGATGCCCGA | |
| | ATGCTAGCGAGCCAGAACATTGTGATCATCCTTTGTGGCAAGAAGGACCTGGATG | |
| | CAGATCGTGAAGTTACCTTCTTAGAAGCCTCCAGATTTGCTCAAGAAAATGAGCTGAT | |
| | GTTTTTGGAAACAAGTGCGCTCACAGGGGAGAATGTAGAAGAGGCTTTTGTACAGTGT | |
| | GCAAGAAAAATACTTAACAAAATCGAATCAGGTGAGCTGGACCCAGAAAGAATGGGCT | |
| | CAGGTATTCAGTACGGAGATGCTGCCTTGAGACAGCTGAGGTCACCGCGGCGCGCACA | |
| | GGCCCCGAACGCTCAGGAGTGTGGTTGTTAGGCGGCCGCTTTTTTCCTT | |
| | ORF Start: at 1 | ORF Stop: TAG at 667 |
| | SEQ ID NO:8 | 222 aa MW at 24790.8 kD |

| | |
|---|---|
| NOV2c, | RGSTMSQTAMSETYDPLFKFLVIGNAGTGKSCLLHQFIEKKFKDDSNHTIGVEFGSKI |
| 209829447 | INVGGKYVKLQIWDTAGQERFRSVTRSYYRGAAGALLVYDITSRETYNALTNWLTDAR |
| Protein | MLASQNIVIILCGNKKDLDADREVTFLEASRFAQENELMFLETSALTGENVEEAFVQC |
| Sequence | ARKILNKTESGELDPERMGSGIQYGDAALRQLRSPRRAQAPNAQECGC |

Sequence comparison of the above protein sequences yields the following, sequence relationships shown in Table 2B.

TABLE 2B
Comparison of NOV2a against NOV2b and NOV2c.

| | | Identities/ |
|---|---|---|
| | NOV2a Residues/ | Similarities for |
| Protein Sequence | Match Residues | the Matched Region |
| NOV2b | 1 . . . 218 | 179/218 (82%) |
| | 1 . . . 180 | 179/218 (82%) |
| NOV2c | 1 . . . 218 | 218/218 (100%) |
| | 5 . . . 222 | 218/218 (100%) |

Further analysis of the NOV2a protein yielded the following properties shown in Table 2C.

TABLE 2C
Protein Sequence Properties NOV2a

| | |
|---|---|
| PSort | 0.6500 probability located in cytoplasm; 0.1000 |
| analysis: | probability located in mitochondrial matrix space; |
| | 0.1000 probability located in lysosome (lumen); |
| | 0.0245 probability located in microbody (peroxisome) |
| SignalP | No Known Signal Sequence Predicted |
| analysis: | |

A search of the NOV2a protein against the Geneseq database, a proprietary database that contains sequences published in patents and patent publication, yielded several homologous proteins shown in Table 2D.

TABLE 2D
Geneseq Results for NOV2a

| | | NOV2a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAB23762 | dRab4 amino acid sequence- | 6 . . . 218 | 186/213 (87%) | e−105 |
| | Unidentified, 213 aa. [CN1257124-A, | 1 . . . 213 | 198/213 (92%) | |
| | 21 Jun. 2000] | | | |
| AAB23763 | rRab4b amino acid sequence- | 6 . . . 218 | 185/213 (86%) | e−105 |
| | Unidentified, 213 aa. [CN1257124-A, | 1 . . . 213 | 197/213 (91%) | |
| | 21 Jun. 2000] | | | |
| AAB23761 | Human Rab4b protein sequence | 6 . . . 218 | 183/213 (85%) | e−103 |
| | SEQ ID NO: 4-Homo sapiens, 213 | 1 . . . 213 | 195/213 (90%) | |
| | aa. [CN1257124-A, 21 Jun. 2000] | | | |
| AAU17547 | Novel signal transduction pathway | 11 . . . 218 | 182/208 (87%) | e−103 |
| | protein, Seq ID 1112-Homo sapiens, | 15 . . . 222 | 193/208 (92%) | |
| | 222 aa. [WO200154733-A1, | | | |
| | 2 Aug. 2001] | | | |
| AAU17127 | Novel signal transduction pathway | 11 . . . 218 | 182/208 (87%) | e−103 |
| | protein, Seq ID 692-Homo sapiens, | 18 . . . 225 | 193/208 (92%) | |
| | 225 aa. [WO200154733-A1, | | | |
| | 2 Aug. 2001] | | | |

In a BLAST search of public sequence databases, the NOV2a protein was found to have homology to the proteins shown in the BLASTP data in Table 2E.

TABLE 2E
Public BLASTP Results for NOV2a

| | | NOV2a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9BQ44 | RAB4, member RAS oncogene family- | 1 . . . 218 | 218/218 (100%) | e−123 |
| | Homo sapiens (Human), 218 aa. | 1 . . . 218 | 218/218 (100%) | |
| P20338 | Ras-related protein Rab-4A-Homo | 6 . . . 218 | 211/213 (99%) | e−119 |
| | sapiens (Human), 213 aa. | 1 . . . 213 | 212/213 (99%) | |
| P56371 | Ras-related protein Rab-4A-Mus | 6 . . . 218 | 208/213 (97%) | e−118 |
| | musculus (Mouse), 213 aa. | 1 . . . 213 | 212/213 (98%) | |
| P05714 | Ras-related protein Rab-4A-Rattus | 6 . . . 218 | 208/213 (97%) | e−117 |
| | norvegicus (Rat), 213 aa. | 1 . . . 213 | 210/213 (97%) | |
| Q9H0Z8 | DJ803J11.1 (RAB4, member RAS | 16 . . . 218 | 198/203 (97%) | e−109 |
| | oncogene family)-Homo sapiens | 1. . . 198 | 198/203 (97%) | |
| | (Human), 198 aa (fragment). | | | |

PFam analysis predicts that the NOV2a protein contains the domains shown in the Table 2F.

TABLE 2F
Domain Analysis of NOV2a

| | | Identities/ | |
|---|---|---|---|
| Pfam | | Similarities for | Expect |
| Domain | NOV2a Match Region | the Matched Region | Value |
| Arf | 5 . . . 177 | 37/199 (19%) | 1.2e−05 |
| | | 106/199 (53%) | |
| Ras | 15 . . . 218 | 88/217 (41%) | 4.1e−91 |
| | | 172/217 (79%) | |

### Example 3

The NOV3 clone was analyzed, and the nucleotide and encoded polypeptide sequences are shown in Table 3A.

TABLE 3A
NOV3 Sequence Analysis

| | SEQ ID NO:9 | 1185 bp |
|---|---|---|
| NOV3a | GTAATATCCTCTCTCCCCCAGATATTAGGAACAGTATCACAGGGGGTGGTACACTCCC | |
| CG112785-01 | TTAGATATTGGGAGTAATATCATCCTCTTGCCTTCTGGATATTAGGAACAATATCCCA | |
| DNA | GAAGGCGTGTACAACCCCCTGCGATACTGGGAGACAGCCCGTCCTCACTGGGCTCTCC | |
| Sequence | CTGTCCATGTACCTGGTCACGATGCTGAGGAACCTGTTCATCATCCTGGCTGGCAGCT | |
| | CTGACCCCCACTTCCACACCCCCATGTACTTCTTCCTCTCCAACCTGTCCTGGGCTGA | |
| | CATTGGTTTCACCTCGGCCACAGTTCCCAAGATGATTGTGGACATGCAGTCGCATAGC | |
| | AGAGTCATCTCTTATGCGGGCTGCCTGACACAGATGTCTTTCTTTGTCCTTTTTGCAT | |
| | GTATAGAAGACATGCTCCTGACTCTGATGGCCTATGACCGATTTGTGGCCATCTGCCA | |
| | CCCCCTGCACTACCGAGTCATCATGAATCCTCACCTCTGTGTCTTCTTAGTTTTGGTG | |
| | TCCTTTTTCCTTAGCCTGTTGGATTCCCAGCTGCACAGCTGGATTGTGTTACAACTCA | |
| | CCTTCTTCAAGAATGTGGAAATCTATAATTTTTTCTGTGACCCATCTCAACTTCTCAA | |
| | TTTGGTTTTCTTCCCATTTCAGGGATCCTTTTGTCTTACTATAAAATTGTCTCCTCCA | |
| | TTCCAAGAATTCCATCGTCAGATGGGAAGTATAAAGCCTTCTCCACCTGTGGCTCTCA | |
| | CCTGGCAGTTGTTTGCTTATTTTATGAAACAGGCATTGGCGTGTACCTGACTTCAGCT | |
| | GTGTCATCATCTCCCAGGAATGGAGTGGTGGCATCAGTGATGTACGCTGTGGTCATCC | |
| | CCATGCTGAACCCTTTCATCTACAGCCTGAGAAACAGGGACATTCATAGTGCCCTGTG | |
| | GAGGCTGCGCAGCAGAACAGTCAAATCTCATGATCTGTTCCATCCTTTCTCTTGTGTG | |
| | AGTAAGAAAGGGCAACCACATTAAATCTGTACATCTGCAAATCCTAACCCCTTTGTCA | |
| | CATTATTTTTGTTGCTTGATGGTTTTATTCCTTTCCACATTTCCTATGTGAATTGCTT | |
| | CTTTGTTATGCCTTTAATGGAATGG | |
| | ORF Start: ATG at 181 | ORF Stop: TAA at 1066 |
| | SEQ ID NO:10 | 295 aa MW at 33372.9 kD |

| | |
|---|---|
| NOV3a, | MYLVTMLRNLFIILAGSSDPHFHTPMYFFLSNLSWADIGFTSATVPKMIVDMQSHSRV |
| CG112785-01 | ISYAGCLTQMSFFVLEACIEDMLLTLMAYDRFVAICHPLHYRVTMNPHLCVFLVLVSF |
| Protein | FLSLLDSQLHSWIVLQLTPFKNVEIYNFFCDPSQLLNLACSDSIINNILCILDIPTFG |
| Sequence | FLPISGTLLSYYKIVSSIPRIPSSDGKYKAFSTCGSHLAVVCLFYETGIGVYLTSAVS |
| | KGQPH |

Further analysis of the NOV3a protein yielded the following properties shown in Table 3B.

TABLE 3B
Protein Sequence Properties NOV3a

| | |
|---|---|
| PSort | 0.6850 probability located in endoplasmic reticulum |
| analysis: | (membrane); 0.6400 probability located in plasma |
| | membrane; 0.4600 probability located in Golgi body; |
| | 0.1000 probability located in endoplasmic reticulum (lumen) |
| SignalP | Cleavage site between residues 19 and 20 |
| analysis: | |

A search of the NOV3a protein against the Geneseq database, a proprietary database that contains sequences published in patents and patent publication, yielded several homolgous proteins shown in Table 3C.

TABLE 3C
Geneseq Results for NOV3a

| | | NOV3a | | |
|---|---|---|---|---|
| | Protein/Organism/ | Residues/ | Identities/ | |
| Geneseq | Length [Patent #, | Match | Similarities for the | Expect |
| Identifier | Date] | Residues | Matched Region | Value |
| AAG72265 | Human olfactory receptor polypeptide, | 1 . . . 286 | 275/290 (94%) | e−156 |
| | SEQ ID NO: 1946-Homo sapiens, 291 | 2 . . . 291 | 278/290 (95%) | |
| | aa. [WO200127158-A2, 19 Apr. 2001] | | | |
| ABG15327 | Novel human diagnostic protein #15318- | 3 . . . 295 | 257/298 (86%) | e−143 |
| | Homo sapiens, 345 aa. [WO200175067- | 48 . . . 345 | 264/298 (88%) | |
| | A2, 11 Oct. 2001] | | | |
| ABG15327 | Novel human diagnostic protein #15318- | 3 . . . 295 | 257/298 (86%) | e−143 |
| | Homo sapiens, 345 aa. [WO200175067- | 48 . . . 345 | 264/298 (88%) | |
| | A2, 11 Oct. 2001] | | | |
| AAU85171 | G-coupled olfactory receptor #32-Homo | 1 . . . 295 | 256/300 (85%) | e−142 |
| | sapiens, 300 aa. [WO200198526-A2, | 1 . . . 300 | 263/300 (87%) | |
| | 27 Dec. 2001] | | | |
| AAE04583 | Human G-protein coupled receptor-39 | 1 . . . 295 | 256/300 (85%) | e−142 |
| | (GCREC-39) protein-Homo sapiens, | 60 . . . 359 | 263/300 (87%) | |
| | 2001] | | | |

In a BLAST search of public sequence databases, the NOV3a protein was found to have homology to the proteins shown in the BLASTP data in Table 3D.

TABLE 3D
Public BLASTP Results for NOV3a

| | | NOV3a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q8VFJ2 | Olfactory receptor MOR145-1- | 1 . . . 276 | 196/276 (71%) | e−112 |
| | Mus musculus (Mouse), 295 aa. | 20 . . . 295 | 228/276 (82%) | |
| O43789 | Olfactory receptor-Homo sapiens | 25 . . . 285 | 200/261 (76%) | e−112 |
| | (Human), 264 aa (fragment). | 1 . . . 260 | 224/261 (85%) | |
| Q9UPJ1 | BC319430_5-Homo sapiens | 26 . . . 285 | 199/260 (76%) | e−111 |
| | (Human), 263 aa. | 1 . . . 259 | 223/260 (85%) | |
| Q8VF19 | Olfactory receptor MOR145-3- | 2 . . . 276 | 178/275 (64%) | e−102 |
| | Mus musculus (Mouse), 295 aa. | 21 . . . 295 | 215/275 (77%) | |
| Q8VFJ0 | Olfactory receptor MOR145-2- | 1 . . . 274 | 183/274 (66%) | e−101 |
| | Mus musculus (Mouse), 319 aa. | 44 . . . 317 | 217/274 (78%) | |

PFam analysis predicts that the NOV3a protein contains the domains shown in the Table 3E.

TABLE 3E
Domain Analysis of NOV3a

| | | Identities/ | |
|---|---|---|---|
| Pfam | | Similarities for | Expect |
| Domain | NOV3a Match Region | the Matched Region | Value |
| 7tm_1 | 8 . . . 257 | 57/277 (21%) | 7.1e−31 |
| | | 186/277 (67%) | |

### Example 4

The NOV4 clone was analyzed and the nucleotide and encoded polypeptide sequences are shown in Table 4A.

TABLE 4A
NOV4 Sequence Analysis

| | SEQ ID NO:11 | 700 bp |
|---|---|---|
| NOV4a, | CTCTAATATGATTCCACCTGTTGGGCTCTTTCTTCCATTTGCCTCCGCAGATAGTGTC | |
| CG116818-02 | TGCCTTCTGGAGAGCTGACCAAACACTAAGGATGCTGAAGTTCCGAACAGTCCATGGG | |
| DNA | GGCCTGAGGCTCCTGGGAATCCGCCGAACCTCCACCGCCCCCGCTGCCTCCCCAAATG | |
| Sequence | TCCGGCGCCTGGAGTATAAGCCCATCAAGAAGTCATGGTGGCCAAACAGAGGTGAGAT | |
| | TGCCATCCGTGTGTTCCGGGCCTGCACGGAGCTGGGCATCCGCACCGTAGCCATCTAC | |
| | TCTGAGCAGGACACGGGCCAGATGCACCGGCAGAAAGCAGATGAAGCCTATCTCATCG | |
| | GCCGCGGCCTGGCCCCCGTGCAGGCCTACCTGCACATCCCAGACATCATCAAGGTGGC | |
| | CAAGGAGAACAACGTAGATGCAGTGCACCCTGGCTACGGGTTCCTTTCTGAGCGAGCG | |
| | AAGGTGATAGACATCAAAGTGGTGGCAGGGGCCAAGGTGGCCAAGGGCCAGCCCCTGT | |
| | GTGTGCTCAGTGCCATGAAGATGGAGACTGTGGTGACCTCACCCATGGAGGGTACTGT | |
| | CCGCAAGGTTCATGTGACCAAGGACATGACACTGGAAGGTGACGACCTCATCCTGGAG | |
| | ATCGAGTGATCTTGCCCCAGACCGGCAGCCTGGCCATCCCCAAGCCTTCAACAGAAGC | |
| | TGTG | |
| | ORF Start: ATG at 90 | ORF Stop: TGA at 645 |
| | SEQ ID N0:12 | 185 aa MW at 20387.8 kD |

| | |
|---|---|
| NOV4a, | MLKFRTVHGGLRLLGIRRTSTAPAASPNVRRLEYKRIKKVMVIAARGETAIRVFRACTE |
| CG116818-02 | LGIRTVAIYSEQDTGQMHRQKADEAYLIGRGLAPVQAYLHIPDITKVAKENNVDAVHP |
| Protein | GYGFLSERAKVIDTKVVAGAKVAKGQPLCVLSAAKMETAATSPMEGTVRKVHVTKDMT |
| Sequence | LEGDDLILETE |

Further analysis of the NOV4a protein yielded the following properties shown in Table 4B.

TABLE 4B
Protein Sequence Properties NOV4a

| | |
|---|---|
| PSort | 0.5964 probability located in mitochondrial matrix space; |
| analysis: | 0.3037 probability located in mitochondrial inner membrane; |
| | 0.3037 probability located in mitochondrial intermembrane |
| | space; 0.3037 probability located in mitochondrial outer |
| | membrane |
| SignalP | Cleavage site between residues 22 and 23 |
| analysis: | |

A search of the NOV4a protein against the Geneseq database a proprietary database that contains sequences published in patents and patent publication yielded several homologous proteins shown in Table 4C.

TABLE 4C
Geneseq Results for NOV4a

| | | | Identities/ | |
|---|---|---|---|---|
| | | | Similari- | |
| | | NOV4a | ties | |
| | Protein/ | Residues/ | for the | |
| Geneseq | Organism/Length | Match | Matched | Expect |
| Identifier | [Patent #, Date] | Residues | Region | Value |
| ABB67309 | Drosophila | 31 . . . 143 | 74/113 | 4e−39 |
| | melanogaster | 33 . . . 145 | (65%) | |
| | polypeptide SEQ | | 94/113 | |
| | ID NO 28719 - | | (82%) | |
| | Drosophila | | | |
| | melanogaster, | | | |
| | 1196 aa. | | | |
| | [WO200171042-A2, | | | |
| | 27 SEP. 2001] | | | |
| ABB66605 | Drosophila | 31 . . . 143 | 74/113 | 4e−39 |
| | melanogaster | 33 . . . 145 | (65%) | |
| | polypeptide SEQ | | 94/113 | |
| | ID NO 26607 - | | (82%) | |
| | Drosophila | | | |
| | melanogaster, | | | |
| | 1181 aa. | | | |
| | [WO200171042-A2, | | | |
| | 27 SEP. 2001] | | | |
| ABB66604 | Drosophila | 31 . . . 143 | 74/113 | 4e−39 |
| | melanogaster | 33 . . . 145 | (65%) | |
| | polypeptide SEQ | | 94/113 | |
| | ID NO 26604 - | | (82%) | |
| | Drosophila | | | |
| | melanogaster, | | | |
| | 1181 aa. | | | |
| | [WO200171042-A2, | | | |
| | 27 SEP. 2001] | | | |
| ABB58211 | Drosophila | 31 . . . 143 | 74/113 | 4e−39 |
| | melanogaster | 33 . . . 145 | (65%) | |
| | polypeptide SEQ | | 94/113 | |
| | ID NO 1425 - | | (82%) | |
| | Drosophila | | | |
| | melanogaster, | | | |
| | 1181 aa. | | | |
| | [WO200171042-A2, | | | |
| | 27 SEP. 2001] | | | |
| AAU00511 | Bacillus subtilis | 32 . . . 123 | 63/92 | 1e−31 |
| | pyruvate | 1 . . . 92 | (68%) | |
| | carboxylase | | 75/92 | |
| | enzyme A - | | (81%) | |
| | Bacillus subtilis | | | |
| | strain 168, 1148 aa. | | | |
| | [EP1092776-A1, | | | |
| | 18 APR. 2001] | | | |

In a BLAST search of public sequence databases, the NOV4a protein was found to have homology to the proteins shown in the BLASTP data in Table 4D.

TABLE 4D
Public BLASTP Results for NOV4a

| | | | Identities/ | |
|---|---|---|---|---|
| | | NOV4a | Similarities | |
| Protein | | Residues/ | for the | |
| Accession | Protein/ | Match | Matched | Expect |
| Number | Organism/Length | Residues | Portion | Value |
| JC2460 | pyruvate carboxylase | 1 . . . 143 | 128/143 (89%) | 3e−66 |
| | (EC 6.4.1.1) | 1 . . . 143 | 129/143 (89%) | |
| | precursor - human, | | | |
| | 1178 aa. | | | |
| P11898 | Pyruvate carboxylase, | 1 . . . 143 | 128/143 (89%) | 3e−66 |
| | mitochondrial | 1 . . . 143 | 129/143 (89%) | |
| | precursor (EC 6.4.1.1) | | | |
| | (Pyruvic carboxylase) | | | |
| | (PCB) - Homo sapiens | | | |
| | (Human), 1178 aa. | | | |
| JC4391 | pyruvate carboxylase | 1 . . . 143 | 121/143 (84%) | 5e−63 |
| | (EC 6.4.1.1) | 1 . . . 143 | 126/143 (87%) | |
| | precursor - rat, | | | |
| | 1178 aa. | | | |
| P52873 | Pyruvate carboxylase, | 1 . . . 143 | 121/143 (84%) | 5e−63 |
| | mitochondrial | 1 . . . 143 | 126/143 (87%) | |
| | precursor | | | |
| | (EC 6.4.1.1) | | | |
| | (Pyruvic carboxylase) | | | |
| | (PCB) - Rattus | | | |
| | norvegicus (Rat), | | | |
| | 1178 aa | | | |
| Q05920 | Pyruvate carboxylase, | 1 . . . 143 | 120/143 (83%) | 2e−62 |
| | mitochondrial | 1 . . . 143 | 126/143 (87%) | |
| | precursor | | | |
| | (EC 6.4.1.1) | | | |
| | (Pyruvic carboxylase) | | | |
| | (PCB) - Mus | | | |
| | musculus (Mouse), | | | |
| | 1178 aa. | | | |

PFam analysis predicts that the NOV4a protein contains the domains shown in the Table 4E.

TABLE 4E
Domain Analysis of NOV4a

| | | Identities/ | |
|---|---|---|---|
| | NOV4a | Similarities | Expect |
| Pfam Domain | Match Region | for the Matched Region | Value |
| CPSase_L_chain | 36 . . . 123 | 38/101 (38%) | 3.5e−29 |
| | | 73/101 (72%) | |
| biotin_lipoyl | 111 . . . 184 | 24/75 (32%) | 1.1e−16 |
| | | 60/75 (80%) | |

### Example 5

The NOV5 clone was analyzed, and the nucleotide and encoded polypeptide sequences are shown in Table 5A.

TABLE 5A
NOV5 Sequence Analysis

| | SEQ ID NO:13 | 2133 bp |
|---|---|---|
| NOV5a, | GAATTCCGGTTTCTTCCTAAAAAATGTCTGATGGCCGCTTTCTCGGTCGGCACCGCCA | |
| CG117653-02 | AATGAATGCCAGCAGTTACTCTGCAGAGATGACGGAGCCCAAGTCGGTGTGTGTCTCGGT | |
| DNA | GGATGAGGTGGTGTCCAGCAACATGGAGGCCACTGAGACGGACCTGCTGAATGGACAT | |
| Sequence | CTGAAAAAAGTAGATAATAACCTCACGGAAGCCCAGCGCTTCTCCTCCTTGCCTCGGA | |
| | GGGCAGCTGTGAACATTGAATTCAGGGACCTTTCCTATTCGGTTCCTGAAGGACCCTG | |
| | GTGGAGGAAGAAAGGATACAAGACCCTCCTGAAAGGAATTTCCGGGAAGTTCAATAGT | |
| | GGTGAGTTGGTGGCCATTATGGGTCCTTCCGGGGCCGGGAAGTCCACGCTGATGAACA | |
| | TCCTGGCTGGATACAGGGAGACGGGCATGAAGGGGGCCGTCCTCATCAACGGCCTGCC | |
| | CCGGGACCTGCGCTGCTTCCGGAAGGTGTCCTGCTACATCATGCAGGATGACATGCTG | |
| | CTGCCGCATCTCACTGTGCAGGAGGCCATGATGGTGTCGGCACATCTGAAGCTTCAGG | |
| | AGAAGGATGAAGGCAGAAGGGAAATGGTCAAGGAGATACTGACAGCGCTGGGCTTGCT | |
| | GTCTTGCGCCAACACGCGGACCGGGAGCCTGTCAGGTGGTCAGCGCAAGCGCCTGGCC | |
| | ATCGCGCTGGAGCTGGTGAACAACCCTCCAGTCATGTTCTTCGATGAGCCCACCAGCG | |
| | GCCTGGACAGCGCCTCCTGCTTCCAGGTGGTCTCGCTGATGAAAGGGCTCGCTCAAGG | |
| | GGGTCGCTCCATCATTTGCACCATCCACCAGCCCAGCGCCAAACTCTTCGAGCTGTTC | |
| | GACCAGCTTTACGTCCTGAGTCAAGGACAATGTGTGTACCGGGGAAAAGTCTGCAATC | |
| | TTGTGCCATATTTGAGGGATTTGGGTCTGAACTGCCCAACCTACCACAACCCAGCAGA | |
| | TTTTGTCATGGAGGTTGCATCCGGCGAGTACGGTGATCAGAACAGTCGGCTGGTGAGA | |
| | GCGGTTCGGGAGGGCATGTGTGACTCAGACCACAAGAGAGACCTCGGGGGTGATGCCG | |
| | AGGTGAACCCTTTTCTTTGGCACCGCCCCTCTGAAGAGGTAAAGCAGACAAAACGATT | |
| | AAAGGGGTTGAGAAAGGACTCCTCGTCCATGGAAGGCTGCCACAGCTTCTCTGCCAGC | |
| | TGCCTCACGCAGTTCTGCATCCTCTTCAAGAGGACCTTCCTCAGCATCATGAGGGACT | |
| | CGGTCCTGACACACCTGCGCATCACCTCGCACATTGGGATCGGCCTCCTCATTGGCCT | |
| | GCTGTACTTGGGGATCGGGAACGAAACCAAGAAGGTCTTGAGCAACTCCGGCTTCCTC | |
| | TTCTTCTCCATGCTGTTCCTCATGTTCGCGGCCCTCATGCCTACTGTTCTGACATTTC | |
| | CCCTGGAGATGGGAGTCTTTCTTCGGGAACACCTGAACTACTGGTACAGCCTGAAGGC | |
| | CTACTACCTGGCCAAGACCATGGCAGACGTGCCCTTTCAGATCATGTTCCCAGTGGCC | |
| | TACTGCAGCATCGTGTACTGGATGACGTCGCAGCCGTCCGACGCCGTGCGCTTTGTGC | |
| | TGTTTGCCGCGCTGGGCACCATGACCTCCCTGGTGGCACAGTCCCTGGGCCTGCTGAT | |
| | CGGAGCCGCCTCCACGTCCCTGCAGGTGGCCACTTTCGTGGGCCCAGTGACAGCCATC | |
| | CCGGTGCTCCTGTTCTCGGGGTTCTTCGTCAGCTTCGACACCATCCCCACGTACCTAC | |
| | AGTGGATGTCCTACATCTCCTATGTCAGGTAGCGGGCGTGGGGCACGCATGGCGTGGG | |
| | GACCGAGCGTGACGGGGGAAGAACCGTCTCCAACAGCGTGAGGGGCTCACAAAAGCCA | |
| | CTCTGGGCTGCTGGCCAAGAGCAGATTACACATCTGAGGATCCAGGCCTTCCATCTTC | |
| | CTGCTAGTTCCACCTCCTCCTACCCTCACCAACACACACACACTAAACAAGGAGGCCA | |
| | CACAAACCAGCGCTTCACACCCGGAGAGCCATGGCAGGACCAAGTGTTCTGGACGTTG | |
| | CCGAGAGCTGCCTTTGGTGGAAGCGCTTCCATCTTTTACGAACGT | |
| | ORF Start: ATG at 31 | ORF Stop: TAG at 1828 |
| | SEQ ID NO:14 | 599 aa MW at 66330.4 kD |

| | |
|---|---|
| NOV5a, | MAAPSVGTAMNASSYSAEMTEPKSVCVSVDEVVSSNMEATETDLLNGHLKKVIDNNLTE |
| CG117653-02 | AQRFSSLPRRAAVNTEFRDLSYSVPEGPWWRKKGYKTLLKGISGKFNSGELVAIMGPS |
| Protein | GAGKSTLMNILAGYRETGMKGAVLINGLPRDLRCFRKVSCYIMQDDMLLPHLTVQEAM |
| Sequence | MVSAHLKLQEKDEGRREMVKETLTALGLLSCAATRTGSLSGGQRKRLATALELVNNPP |
| | AAVMFFDEPTSGLDSASCFQVVSLMKGLAQGGRSITCTTHQRSAKLPELFDQLYVLSQGQ |
| | CVYRGKVCNLVPYLRDLGLNCPTYHNPADFVMEVASGEYGDQNSRLVRAVREGMCDSD |
| | HKRDLGGDAEVNPFLWHRPSEEVKQTKRLKGLRKDSSSMEGCHSFSASCLTQFCILFK |
| | RTFLSIMRDSVLTHLRTTSHIGTGLLIGLLYLGIGNETKKVLSNSGFLFFSMLFLMFA |
| | ALMPTVLTFPLEMGVFLREHLNYWYSLKAYYLAKTMADVPFQIMPPVAYCSIVYWMTS |
| | QPSDAVRPVLFAALGTMTSLVAQSLGLLIGAASTSLQVATFVGPVTAIPVLLFSGFFV |
| | ISFDTTPTYLQWMSYISYVR |

Further analysis of the NOV5a protein yielded the following, properties shown in Table 5B.

TABLE 5B
Protein Sequence Properties NOV5a

| | |
|---|---|
| PSort | 0.6000 probability located in plasma membrane; 0.5876 |
| analysis: | probability located in mitochondrial inner membrane; 0.4000 |
| | probability located in Golgi body; 0.3000 probability located |
| | in endoplasmic reticulum (membrane) |
| SignalP | No Known Signal Sequence Predicted |
| analysis: | |

A search of the NOV5a protein against the Geneseq database, a proprietary database that contains sequences published in patents and patent publication, yielded several homologous proteins shown in Table 5C.

TABLE 5C
Geneseq Results for NOV5a

| | | | Identities/ | |
|---|---|---|---|---|
| | | | Similari- | |
| | | NOV5a | ties | |
| | Protein/ | Residues/ | for the | |
| Geneseq | Organism/Length | Match | Matched | Expect |
| Identifier | [Patent #, Date] | Residues | Region | Value |
| ABB57112 | Mouse ischaemic | 1 . . . 599 | 566/599 | 0.0 |
| | condition related | 5 . . . 591 | (94%) | |
| | protein sequence | | 577/599 | |
| | SEQ ID NO. 255 - | | (95%) | |
| | Mus musculus, | | | |
| | 666 aa. | | | |
| | [WO200188188-A2, | | | |
| | 22 NOV. 2001] | | | |
| AAO14186 | Human transporter | 38 . . . 599 | 406/562 | 0.0 |
| | and ion channel | 26 . . . 572 | (72%) | |
| | TRICH-3 - Homo | | 465/562 | |
| | sapiens, 646 aa. | | (82%) | |
| | [WO200204520-A2, | | | |
| | 17 JAN. 2002] | | | |
| ABB61867 | Drosophila | 58 . . . 599 | 243/550 | e−125 |
| | melanogaster | 90 . . . 614 | (44%) | |
| | polypeptide | | 343/550 | |
| | SEQ ID | | (62%) | |
| | NO 12393 - | | | |
| | Drosophila | | | |
| | melanogaster, | | | |
| | 689 aa. | | | |
| | [WO200171042-A2, | | | |
| | 27 SEP. 2001] | | | |
| AAM00994 | Human bone | 92 . . . 418 | 221/327 | e−119 |
| | marrow | 19 . . . 322 | (67%) | |
| | protein, SEQ ID | | 255/327 | |
| | NO: 495 - | | (77%) | |
| | Homo sapiens, | | | |
| | 935 aa. | | | |
| | [WO200153453-A2, | | | |
| | 26 JUL. 2001] | | | |
| ABB59648 | Drosophila | 190 . . . 599 | 213/412 | e−116 |
| | melanogaster | 150 . . . 546 | (51%) | |
| | polypeptide | | 291/412 | |
| | SEQ ID NO 5736 - | | (69%) | |
| | Drosophila | | | |
| | A2, 27 SEP. 2001] | | | |

In a BLAST search of public sequence databases, the NOV5a protein was found to have homology to the proteins shown in the BLASTP data in Table 5D.

TABLE 5D
Public BLASTP Results for NOV5a

| | | | Identities/ | |
|---|---|---|---|---|
| | | NOV5a | Similarities | |
| Protein | | Residues/ | for the | |
| Accession | Protein/ | Match | Matched | Expect |
| Number | Organism/Length | Residues | Portion | Value |
| P45844 | ATP-binding | 1 . . . 599 | 598/599 (99%) | 0.0 |
| | cassette, sub- | 5 . . . 603 | 598/599 (99%) | |
| | family G, | | | |
| | member 1 (White | | | |
| | protein homolog) | | | |
| | (ATP-binding | | | |
| | cassette | | | |
| | transporter 8) - | | | |
| | Homo sapiens | | | |
| | (Human), 678 aa. | | | |
| AAH29158 | Hypothetical 73.7 | 1 . . . 599 | 586/599 (97%) | 0.0 |
| | kDa protein - | 1 . . . 587 | 586/599 (97%) | |
| | Homo sapiens | | | |
| | (Human), 662 aa. | | | |
| Q9EPG9 | ABC transporter, | 1 . . . 599 | 566/599 (94%) | 0.0 |
| | white | 5 . . . 591 | 578/599 (96%) | |
| | homologue - | | | |
| | Rattus norvegicus | | | |
| | (Rat), 666 aa. | | | |
| Q64343 | ATP-binding | 1 . . . 599 | 566/599 (94%) | 0.0 |
| | cassette, | 5 . . . 591 | 577/599 (95%) | |
| | sub-family G, | | | |
| | member 1 | | | |
| | (White protein | | | |
| | homolog) | | | |
| | (ATP-binding | | | |
| | cassette | | | |
| | transporter 8)- | | | |
| | Mus musculus | | | |
| | (Mouse), 666 aa. | | | |
| G02068 | white homolog - | 37 . . . 599 | 561/563 (99%) | 0.0 |
| | human, 638 aa. | 1 . . . 563 | 561/563 (99%) | |

PFam analysis predicts that the NOV5a protein contains the domains shown in the Table 5E.

TABLE 5E
Domain Analysis of NOV5a

| | | Identities/ | |
|---|---|---|---|
| | NOV5a | Similarities | Expect |
| Pfam Domain | Match Region | for the Matched Region | Value |
| PRK | 109 . . . 124 | 7/16 (44%) | 0.37 |
| | | 13/16 (81%) | |
| GBP | 110 . . . 129 | 13/20 (65%) | 0.11 |
| | | 16/20 (80%) | |
| ABC_tran | 107 . . . 289 | 70/201 (35%) | 1.9e−41 |
| | | 143/201 (71%) | |

### Example 6

The NOV6 clone was analyzed, and the nucleotide and encoded polypeptide sequences are shown in Table 6A.

TABLE 6A
NOV6 Sequence Analysis

| | SEQ ID NO:15 | 1940 bp |
|---|---|---|
| NOV6a, | CCAGAGAGTCTGTGTGAGATGAAGACAGAGGCCCAGCCTTCGACATCCTTGCTGGCAA | |
| CG119674-02 | ACACCTCATGGACTGGCACAGTGATTTCTGACAGTGTCCCAGGAAGTCAAACGTGGGA | |
| DNA | AGACAAGGGTTCATTGACCCGGCCTGCAACATCTCGGACCTCAGAGGCCCAAGTTTCA | |
| Sequence | GCAGCCCGGGTTGCAGAGGCTCAGGCCAGGACCAGTCAGCCCAAGCAAATTTCTGTAT | |
| | TGGAGGCGTTAACTGCCTCAGCCCTGAACCAGAAACCCACGCATGAGAAGGTGCAGAT | |
| | GACAGAGAAGAAAGAGAGTGAGGCAGTTTCGCTGCCATCTACATCTTCATGCTGTTCC | |
| | TGGTCGGGGTTCCTCTTCTCTTCCTGGAGATGGCAGCTGGTCAGAGCATGCGTCAGGG | |
| | TGGCATGGGTGTATGGAAGATCATTGCCCCCTGGATTGGTGGTGTGGGGTATTCTAGC | |
| | TTCATGGAATGCTGAAATACTTTTAAAGCTGATAAACCTAGGGAAACTGCCTCCTGAT | |
| | GCCAAGCCCCCTGTCAACCTGCTTTACAACCCAACCTCCATCTACAATGCCTGGCTCA | |
| | GTGGCCTTCCCCAGCACATCAAAAGCATGGTTCTCCGCGAGGTGACTGAGTGCAACAT | |
| | AGAGACTCAGTTTCTTAAGGCTAGCGAGGGCCCAAAGTTTGCATTCCTGTCCTTTGTT | |
| | GAAGCCATGTCCTTCCTTCCTCCGTCTGTCTTCTGGTCTTTTATCTTCTTCCTGATGT | |
| | TGCTGGCCATGGGGCTGAGCAGCGCAATAGGGATTATGCAGGGCATCATTACTCCACT | |
| | CCAGGACACCTTCTCTTTCTTCAGGAAACATACAAAGCTGCTCATAGTGGGAGTCTTT | |
| | TTGCTCATGTTCGTGTGCGGCCTCTTCTTCACTCGACCTTCAGGCAGCTACTTCATCA | |
| | GACTGCTGAGTGACTACTGGATAGTCTTCCCCATCATCGTCGTTGTCGTATTTGAAAC | |
| | CATGGCTGTATCCTGGGCCTATGGGGCCAGGAGGTTCCTTGCAGACCTGACGATCCTG | |
| | TTGGGCCACCCCATCTCTCCCATCTTTGGTTGGCTGTGGCCCCATCTGTGTCCAGTTG | |
| | TGCTGCTAATCATCTTTGTGACCATGATGGTTCATCTTTGTATGAAGCCGATTACCTA | |
| | CATGTCCTGGGACTCAAGCACCTCPAAAGAGGTGCTTCGACCATACCCACCGTGGGCA | |
| | CTGCTCTTGATGATCACCCTTTTTGCCATTGTCATCCTCCCCATCCCTGCATACTTTG | |
| | TATACTGCCGCATACATAGGATTCCCTTCAGGCCCAAGAGCGGAGACGGGCCTATGAC | |
| | AGCCTCCACATCCCTACCCCTAAGTCACCAGCTAACACCCAGTAAAGAGGTTCAAAAG | |
| | GAAGAAATTCTACAAGTTGATGAAACAAAGTACCCATCAACTTGTAATGTGACTTCCT | |
| | AACTTCATTAATTTGGCTTCACATAACATATCCCTTAGAACAGATCCAATAGACAACT | |
| | CTTAATATCAGCTTGCAACTGTTGATCTCCCTGGATCCAGAACCACTTTTATTTCCAA | |
| | GAGGAGGGGCATTCTTTGGGGCTGTTCATGGGGCCTGGACTTGCAATCCCTTCCTGGG | |
| | TCCCATCTTACCTGGTGACCACCATCATTGTTTTCCCCATCCTCTTCCTCAACACACA | |
| | TACATGCACAACACATATACAATACTAGTGATGTCTACCAGTCCTGCTACTTCTGGGG | |
| | TGCCTGTCTCCTGGAATGGAGCTGGAGGAGCAATGCTGTTGGTGAATAAATCAGTCTA | |
| | CTGGAACTCCAAGGACTGGATGTAAGCAGATCTTTTTTTCCTATAGATGTCTCAGATG | |
| | TTCAGTTTTCCTGTCACAAGGCTTCCAGTCTGTATTAGTTCATTTTCACACTGATAAT | |
| | ACAGACATACCTGAAACTGGGAAAAA | |
| | ORF Start: ATG at 19 | ORF Stop: TAA at 1450 |
| | SEQ ID NO:16 | 477 aa MW at 53345.0 kD |
| NOV6a, | MKTEAQPSTSLLANTSWTGTVISDSVPGSQTWEDKGSLTRPATSRTSEAQVSAARVAE | |
| CG119674-02 | AQARTSQPKQISVLEALTASALNQKRTHEKVQMTEKKESEAVSLPSTSSCCSWSGFLF | |
| Protein | SSWRWQLVPACVRVAWVYGRSLPRGLVVWGTLASWNAEILLKLINLGKLPPDAKPPVN | |
| Sequence | AALLYNPTSIYNAWLSGLPQHIKSMVLREVTECNIETQFLKASEGPKFAFLSFVEAMSFL | |
| | PPSVFWSFIFFLMLLAMGLSSAIGIMQGIITPLQDTFSFFRKRTKLLIVGVFLLMFVC | |
| | GLFFTRPSGSYFTRLLSDYWIVFPIIVVVVFETMAVSWAYGARRFLADLTILLGHPIS | |
| | PIFGWLWPHLCPVVLLIIFVTMMVHLCMKPITYMSWDSSTSKEVLRPYPPWALLLMITI | |
| | LFAIVTLPTPAYFVYCRIHRTPFRPKSGDGPMTASTSLPLSHQLTPSKEVQKEEILQV | |
| | DETKYPSTCNVTS | |
| | SEQ ID NO:17 | 1904 bp |
| NOV6b, | CCAGAGAGTCTGTGTGAGATGAAGACAGAGGCCCAGCCTTCGACATCCTTGCTGGCAA | |
| CG119674-03 | ACACCTCATCGACTGGCACAGTGATTTCTGACAGTGTCCCAGGAAGTCAAACGTGGGA | |
| DNA | AGACAAGGGTTCATTGACCCGGTCTGCAACATCTTGGACCTCAGAGGCCCAAGTTTCA | |
| Sequence | GCAGCCCGGGTTGCAGAGGCTCAGGCCAGGACCAGTCAGCCCAAGCAAATTTCTGTAT | |
| | TGGGCGCGTTAACTGCCTCAGCCCTGAACCAGAAACCCACGCATGAGAAGGTGCAGAT | |
| | GAGTATATTCTGGCTCAGGCAGTTTCGCTGCCATCTACATCTTCATGCTGTTCCTGGT | |
| | CGGGGTTCCTCTTCTCTTCCTGGAGATGGCAGCTGGTCAGAGCATGCGTCAGGGTGGC | |
| | ATGGGTGTATGGAAGATCATTGCCCCCTGGATTGGTGGTGTGGGGTATTCTAGCTTCA | |
| | TGGAATGCTGAAATACTTTTAAAGCTGATAAACCTAGGGAAACTGCCTCCTGATGCCA | |
| | AGCCCCCTGTCAACCTGCTTTACAACCCAACCTCCATCTACAATGCCTGGCTCAGTGG | |
| | CCTTCCCCAGCACATCAPAAGCATGGTTCTCCGCGAGGTGACTGAGTGCAACATAGAG | |
| | ACTCAGTTTCTTAAGGCTAGCGAGGGCCCAAAGTTTGCATTCCTGTCCTTTGTTGAAG | |
| | CCATGTCCTTCCTTCCTCCGTCTGTCTTCTGGTCTTTTATCTTCTTCCTGATGTTGCT | |
| | GGCCATGGGGCTGAGCAGCGCAATAGGGATTATGCAGGGCATCATTACTCCACTCCAG | |
| | GACACCTTCTCTTTCTTCAGGAAACATACAAAGCTGCTCATAGTGGGAGTCTTTTTGC | |
| | TCATGTTCGTGTGCGGCCTCTTCTTCACTCGACCTTCAGGCAGCTACTTCATCAGACT | |
| | GCTGAGTGACTACTGGATAGTCTTCCCCATCATCGTCGTTGTCGTATTTGAAACCATG | |
| | GCTGTATCCTGGGCCTATGGGGCCAGGAGGTTCCTTGCAGACCTGACGATCCTGTTGG | |
| | GCCACCCCATCTCTCCCATCTTTGGTTGGCTGTGGCCCCATCTGTGTCCAGTTGTGCT | |
| | GCTAATCATCTTTGTGACCATGATGGTTCATCTTTGTATGAAGCCGATTACCTACATG | |
| | TCCTGGGACTCAAGCACCTCAAAAGAGGTGCTTCGACCATACCCACCGTGGGCACTGC | |
| | TCTTGATGATCACCCTTTTTGCCATTGTCATCCTCCCCATCCCTGCATACTTTGTATA | |
| | CTGCCGCATACATAGGATTCCCTTCAGGCCCAAGAGCGGAGACGGGCCTATGACAGCC | |
| | TCCACATCCCTACCCCTAAGTCACCAGCTAACACCCAGTAAAGAGGTTCAAAAGGAAG | |
| | AAATTCTACAAGTTGATGAAACAAAGTACCCATCAACTTGTAATGTGACTTCCTAACT | |
| | TCATTAATTTGGCTTCACATAACATATCCCTTAGAACAGATCCAATAGACAACTCTTA | |
| | ATATCAGCTTGCAACTGTTGATCTCCCTGGATCCAGAACCACTTTTATTTCCAAGAGG | |
| | AGGGGCATTCTTTGGGGGTGTTCATGGGGCCTGGACTTGCAATCCCTTCCTGGGTCCC | |
| | ATCTTACCTGGTGACCACCATCATTGTTTTCCCCATCCTCTTCCTCAACACACATACA | |
| | TGCACAACACATATACAATACTAGTGATGTCTACCAGTCCTGCTACTTCTGGGGTGCC | |
| | TGTCTCCTGGAATGGAGCTGGAGGAGCAATGCTGTTGGTGAATAAATCAGTCTACTGG | |
| | AACTCCAAGGACTGGATGTAAGCAGATCTTTTTTTCCTATAGATGTCTCAGATGTTCA | |
| | GTTTTCCTGTCACAAGGCTTCCAGTCTGTATTAGTTCATTTTCACACTGATAATACAG | |
| | ACATACCTGAAACTGGGAAAAA | |
| | ORF Start: ATG at 19 | ORF Stop: TAA at 1504 |
| | SEQ ID NO:18 | 495 aa MW at 55397.4 kD |

| | |
|---|---|
| NOV6b, | MKTEAQPSTSLLANTSWTGTVISDSVRGSQTWEDKGSLTRSATSWTSEAQVSAARVAE |
| CG119674-03 | AAAQARTSQPKQTSVLGALTASALNQKPTHEKVQMTEKKESEVLLARPFWSSKTEYILAQ |
| Protein | AVSLPSTSSCCSWSGPLFSSWRWQLVRACVRVAWVYGRSLPPGLVVWGTLASWNAEIL |
| Sequence | AALKLINLGKLPPDAKPPVNLLYNPTSIYNAWLSGLPQHIKSMVLREVTECNIETQPLKA |
| | AASEGPKFAFLSFVEANSFLPPSVFWSFIFFLMLLAMGLSSAIGIMQGTITPLQDTPSFF |
| | IRKHTKLLIVGVFLLMFVCGLFFTRPSGSYFIRLLSDYWIVFPIIVVVVFETMAVSWAY |
| | AAGARRFLADLTILLGHPISPTFGWLWPHLCPVVLLIIPVTMMVHLCMKPITYMSWDSST |
| | ISKEVLRPYPPWALLLMITLPAIVILPIPAYFVYCRIRRIPFRPKSGDGPMTASTSLPL |

Sequence comparison of the above protein sequences yields the following, sequence relationships shown in Table 6B.

TABLE 6B
Comparison of NOV6a against NOV6b.

| | NOV6a Residues/ | Identities/Similarities |
|---|---|---|
| Protein Sequence | Match Residues | for the Matched Region |
| NOV6b | 1 . . . 477 | 451/495 (91%) |
| | 1 . . . 495 | 451/495 (91%) |

Further analysis of the NOV6a protein yielded the following, properties shown in Table 6C.

TABLE 6C
Protein Sequence Properties NOV6a

| | |
|---|---|
| PSort analysis: | 0.6000 probability located in plasma membrane; 0.4000 |
| | probability located in Golgi body; 0.3777 probability |
| | located in mitochondrial inner membrane; 0.3000 |
| | probability located in endoplasmic reticulum |
| | (membrane) |

Signal analysis: No Known Signal Sequence Predicted

A search of the NOV6a protein against the Geneseq database, a proprietary database that contains sequences published in patents and patent publication, yielded several homologous proteins shown in Table 6D.

TABLE 6D
Geneseq Results for NOV6a

| | | NOV6a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| ABG16783 | Novel human | 1 . . . 100 | 96/100 (96%) | 3e−46 |
| | diagnostic protein | 440 . . . 539 | 97/100 (97%) | |
| | #16774 - Homo | | | |
| | sapiens, 610 aa. | | | |
| | [WO200175067- | | | |
| | A2, | | | |
| | 11 OCT. 2001] | | | |
| ABG16783 | Novel human | 1 . . . 100 | 96/100 (96%) | 3e−46 |
| | diagnostic protein | 440 . . . 539 | 97/100 (97%) | |
| | #16774 - Homo | | | |
| | sapiens, 610 aa. | | | |
| | [WO200175067- | | | |
| | A2, | | | |
| | 11 OCT. 2001] | | | |
| AAE21800 | Human HIPHUM | 152 . . . 471 | 103/325 (31%) | 4e−44 |
| | 0000029 protein - | 370 . . . 682 | 166/325 (50%) | |
| | Homo sapiens, | | | |
| | 727 aa. | | | |
| | [GB2365432-A, | | | |
| | 20 FEB. 2002] | | | |
| ABB77168 | Human GABA | 152 . . . 427 | 92/278 (33%) | 5e−44 |
| | transporter | 371 . . . 645 | 153/278 (54%) | |
| | protein - Homo | | | |
| | sapiens, 730 aa. | | | |
| | [U.S. Pat. No. | | | |
| | 2002031800-A1, | | | |
| | 14 MAR. 2002] | | | |
| AAE14404 | Human | 152 . . . 427 | 92/278 (33%) | 5e−44 |
| | neurotransmitter | 371 . . . 645 | 153/278 (54%) | |
| | transporter, | | | |
| | NTT-2 - | | | |
| | Homo sapiens, | | | |
| | 730 aa. | | | |
| | [WO200190148- | | | |
| | A2, | | | |
| | 29 NOV. 2001] | | | |

In a BLAST search of public sequence databases, the NOV6a protein was found to have homolog,y to the proteins shown in the BLASTP data in Table 6E.

TABLE 6E
Public BLASTP Results for NOV6a

| | | | Identities/ | |
|---|---|---|---|---|
| | | NOV6a | Similarities | |
| Protein | | Residues/ | for the | |
| Accession | Protein/ | Match | Matched | Expect |
| Number | Organism/Length | Residues | Portion | Value |
| Q9GZN6 | Orphan sodium- | 152 . . . 477 | 326/326 (100%) | 0.0 |
| | and chloride- | 411 . . . 736 | 326/326 (100%) | |
| | dependent | | | |
| | neurotransmitter | | | |
| | transporter | | | |
| | NTT5 - Homo | | | |
| | sapiens (Human), | | | |
| | 736 aa. | | | |
| I52632 | sodium-dependent | 150 . . . 427 | 99/281 (35%) | 1e−45 |
| | neurotransmitter | 370 . . . 646 | 160/281 (56%) | |
| | transporter - | | | |
| | rat, 730 aa | | | |
| | (fragment). | | | |
| Q08469 | Orphan sodium- | 150 . . . 427 | 99/281 (35%) | 1e−45 |
| | and chloride- | 369 . . . 645 | 160/281 (56%) | |
| | dependent | | | |
| | neurotransmitter | | | |
| | transporter | | | |
| | NTT73 | | | |
| | (Orphan | | | |
| | transporter | | | |
| | v7-3) - Rattus | | | |
| | norvegicus | | | |
| | (Rat), 729 aa. | | | |
| I65413 | sodium-dependent | 150 . . . 427 | 99/281 (35%) | 2e−45 |
| | neurotransmitter | 368 . . . 644 | 160/281 (56%) | |
| | transporter - rat, | | | |
| | 728 aa | | | |
| | (fragment). | | | |
| Q9XS59 | Orphan sodium- | 152 . . . 427 | 95/279 (34%) | 2e−44 |
| | and chloride- | 371 . . . 645 | 158/279 (56%) | |
| | dependent | | | |
| | neurotransmitter | | | |
| | transporter | | | |
| | NTT73 (Orphan | | | |
| | transporter v7-3) - | | | |
| | Bos taurus | | | |
| | (Bovine), 729 aa. | | | |

PFam analysis predicts that the NOV6a protein contains the domains shown in the Table 6F.

TABLE 6F
Domain Analysis of NOV6a

| | | Identities/ | |
|---|---|---|---|
| | NOV6a | Similarities | Expect |
| Pfam Domain | Match Region | for the Matched Region | Value |
| SNF | 205 . . . 425 | 82/225 (36%) | 5.2e−40 |
| | | 160/225 (71%) | |

### Example 7

The NOV7 clone was analyzed, and the nucleotide and encoded polypeptide sequences are shown in Table 7A.

TABLE 7A
NOV7 Sequence Analysis

| | SEQ ID NO:19 | 4795 bp |
|---|---|---|
| NOV7a, | ACAGCCGCGCGACGCCGCCGCCTTAGAACGCCTTTCCAGTACTGCTAGCAGCAGCCCG | |
| CG120123-02 | ACCACGCGTTACCGCACGCTCGCGCCTTTCCCTTGACACGGCGGACGCCGGAGGATTG | |
| DNA | GGGCGGCAATTTGTCTTTTCCTTTTTTATTAAAATTATTTTTCCTcSCCTGTTGTTGGA | |
| Sequence | TTTGGGGAAATTTTTTGTTTGTTTTTTATGATTTGTATTTGACTGAGAGAAACCCACT | |
| | GAAGACGTCTGCGTGAGAATAGAGACCACCGAGGCCGACTCGCGGGCCGCTGCACCCA | |
| | CCGCCAAGGACAAAAGGAGCCCAGCGCTACTAGCTGCACCCGATTCCTCCCAGTGCTT | |
| | AGCATGAAGAAGGCCGAAATGGGACGATTCAGTATTTCCCCGGATGAAGACAGCAGCA | |
| | GCTACAGTTCCAACAGCGACTTCAACTACTCCTACCCCACCAAGCAAGCTGCTCTGAA | |
| | AAGCCATTATGCAGATGTAGATCCTGAAAACCAGAACTTTTTACTTGAATCGAATTTG | |
| | GGGAAGAAGAAGTATGAAACAGAATTTCATCCAGGTACTACTTCCTTTGGAATGTCAG | |
| | TATTTAATCTGAGCAATGCGATTGTGGGCAGTGGAATCCTTGGGCTTTCTTATGCCAT | |
| | GGCTAATACTGGAATTGCTCTTTTTATAATTCTCTTGACATTTGTGTCAATATTTTCC | |
| | CTGTATTCTGTTCATCTCCTTTTGAAGACTGCCAATGAAGGAGGGTCTTTATTATATG | |
| | AACAATTGGGATATAAGGCATTTGGATTAGTTGGAAAGCTTGCAGCATCTGGATCAAT | |
| | TACAATGCAGAACATTGGAGCTATGTCAAGCTACCTCTTCATAGTGAAATATGAGTTG | |
| | CCTTTGGTGATCCAGGCATTAACGAACATTGAAGATAAAACTGGATTGTGGTATCTGA | |
| | ACGGGAACTATTTGGTTCTGTTGGTGTCATTGGTGGTCATTCTTCCTTTGTCGCTGTT | |
| | TAGAAATTTAGGATATTTGGGATATACCAGTGGCCTTTCCTTGTTGTGTATGGTGTTC | |
| | TTTCTGATTGTGGTCATTTGCAAGAAATTTCAGGTTCCGTGTCCTGTGGAAGCTGCTT | |
| | TGATAATTAACGAAACAATAAACACCACCTTAACACAGCCAACAGCTCTTGTACCTGC | |
| | TTTGTCACATAACGTGACTGAAAATGACTCTTGCAGACCTCACTATTTTATTTTCAAC | |
| | TCACAGACTGTCTATGCTGTGCCAATTCTGATCTTTTCATTTGTCTGTCATCCTGCTG | |
| | TTCTTCCCATCTATGAAGAACTGAAAGACCGCAGCCGTAGAAGAATGATGAATGTGTC | |
| | CAAGATTTCATTTTTTGCTATGTTTCTCATGTATCTGCTTGCCGCCCTCTTTGGATAC | |
| | CTAACATTTTACGAACATGTTGAGTCAGAATTGCTTCATACCTACTCTTCTATCTTGG | |
| | GAACTGATATTCTTCTTCTCATTGTCCGTCTGGCTGTGTTAATGGCTGTGACCCTGAC | |
| | AGTACCAGTAGTTATTTTCCCAATCCGGAGTTCTGTAACTCACTTGTTGTGTGCATCA | |
| | AAAGATTTCAGTTGGTGGCGTCATAGTCTCATTACAGTGTCTATCTTGGCATTTACCA | |
| | ATTTACTTGTCATCTTTGTCCCAACTATTAGGGATATCTTTGGTTTTATTGGTGCATC | |
| | TGCAGCTTCTATGTTGATTTTTATTCTTCCTTCTGCCTTCTATATCAAGTTGGTGAAG | |
| | AAAGAACCTATGAAATCTGTACAAAAGATTGGGGCTTTGTTCTTCCTGTTAAGTGGTG | |
| | TACTGGTGATGACCGGAAGCATGGCCTTGATTGTTTTGGATTGGGTACACAATGCACC | |
| | TGGAGGTGGCCATTAATGGCACCACTCAAACTCAAACTCAGTCCATCTGATGCCAGT | |
| | GTTGAGTAAACTCAACTACTATGAAATTTCACCTAATGTTTTCAGTTTCACTTCCTTT | |
| | TGAAGTGCAGATTCCTCGCTGGTTCTTCTGAGTGCAGAATAAGTGAACTTTTTTGTTT | |
| | TGTTTTGTTTTTTTAAGAAACTTATCTGTATGTTAGAAATGGATATGAACAACAAAAC | |
| | CACGAGTCTCGGGTTAAGGGAAGTGACAATTTTATTCCCATTCCAGAGAATGGACAAA | |
| | CTCTTAACTTTTATCAAGCCACATGCTTGGCTGTGTCATTGTTTAACTTGGATATTTT | |
| | ATGATTTTACTTGAATGTGCCTAATGGAACCATTTGATGTGAGAAACAATTCTTTTTA | |
| | ATTTACAGCAAAATATTGAATAACCATTGACAAAAACACTATTATTTTTTGTACCAAA | |
| | AATACTTAAAGACCTCAGAAGCACTCTTTTACTTTTAAGAAATTGCTTTTTTGAACTT | |
| | TATTCAGAAGCAGTTATCAATAAATTCCATAAAATAATGTCATTGGTATTTAAAAATG | |
| | AATATTAATATAATGAAATGGTTTGCCTTTTTGTAGGCATAATAAGCCAAATACTTTT | |
| | TTACCCAAAATAATTTTTAGAGAAAATGATGTAATGAAAAATTGTACCATGAATTAGG | |
| | AGCATAGTTTTTTCCATTTAAACGTCACCATTACTTAAAAGATGATTGATTATTGCTA | |
| | TACCAAATCAGATGAACTCTGTTCATCACTTTTCTTCTCTGTCCCCAAACAATTTGGT | |
| | TCATTCAGACTGAAATGTTTGTGTCTTCAACTTATTAGAATGGAAGATAATGCAGATA | |
| | TTTCTGTGGGAAATAAAATAACTAATTTTGAGGTACCAAATAGTGCAATTGGGTAAAA | |
| | CAGGGTTTATTCAGTTGCATCTGTCTCCAGTGTTGTATTGACAGCTCTGGGTCTTTTT | |
| | TTGGGCCAGCCCTTTTTTGACATTGCTTCCAGCAGTGGAAAATGGGCATTTGATGGCA | |
| | ATAGGCCAAAATTATTGTGTCCAGAGAGTACACTTTTTCAAAATGCTCACCTACTGGA | |
| | AGTGTGAATTACTTGACAATGTATGGCTTAGTTGTGTTCATGTTTTGTCTACAGTAGA | |
| | GGTCTAATCCACAGGTTACACCTATGTTTGATATGATATAAGTTCTCTTTGCGTAGGC | |
| | CACTGGGTTTCTCATGCAGTAAGCTTTATAAAAACTCATTTGCACTGGACTGTCATCT | |
| | CATTCTTGTACAACGTAGAATTACTTGTTTACATCCAACAAATGGTTAGCTAGGGAAA | |
| | ACAGTGCAAACTGAGTGTTAGTAGTCATTTTGGTCCAACTGCATGTCAACCCTTCCAT | |
| | TATCGTACGTCACAGTGTATGGTGAATATATTATTAAATAATGTGGTACTTCGCTCAT | |
| | CAGGCATAATGTCTAAAATCTAATATACATAATTCCATTAAGTGGTTGAAGGAAGCAA | |
| | ATAATGGAATTGTCAATTGGTCATCTGGCTGTAAGGTTTGCCCTTGAACTAAAAATGT | |
| | TGTTTGGGGCAAGGGCCAGAAATGTGGAGACATGGTTTTTGTTACGCATTCTTGTATT | |
| | ATATGTGACTAAATTTACAAACAAGATACATGTGTAATTAAAGACCCTTATGGAACTG | |
| | GAAGACGTCTTGTAGTGCTACATTGCAGTGAAACCGTTGGTCCATTTTTGTCCTGTTTC | |
| | TATGAAGATAAAATAATTGGGGGCCATCTAGAAATAGAAAGGCAGTGGGAAGACAGAT | |
| | TCTACGGCACTGCTTTCATTTAATTGGGCTTTAGGCACTCCATTCGAATGCAGAACCT | |
| | CACCTCTAGTTGAGACCAAGAATTGGCAAATTTGCATGAGCTCCTGGAAAGAGTTGCT | |
| | GACTTTGTATCTAAGACCTGCCAGGGAATACCAAGAGTTGTTTCTACAGACTTTTTTT | |
| | TTTTTTTTGTATGGGAGAAGATACTGTGGCAACCAGGAAGGAATGGAAAAAAAATTCT | |
| | TTTCTCTACAGCAAATTAATGTGAGGAAGCTCCTCCAATCCTCTGGCTATTTAAGGTT | |
| | CAAAATCAAGTGCCTAGGGAAAATTCCAATGGATGATTTTCTGGGAGCTATCTTGTCT | |
| | ACCTTGAGGTTCCTGAACAATGAATTCCCATTAATGAGCAGTCTTCAGTATTAAAACC | |
| | ACTGTCTTGTCACCTCATTTTGCATTACTGTCTTCCGTGGATGTTTCAGTTACAACTG | |
| | TAATGTTATTTATAGAACAACATTAATCCATTAAAGCTAACCTATTTTTCAATATTTA | |
| | TGATAATCTATGTACATATATTGTCTGTCCATATGTATTTGTAAATAGGTTGTATATA | |
| | ATGTCAGGTTTGGGTCTTGGGTTCAAGTGTATATATTCCTGTAAGTTTCTTAACTGCA | |
| | TTTTGATGAATTCACATTATGTAACTATAAGAATTGTCCCAAAAGTACCTGTACAGAA | |
| | AATTGAATATTGAAAAATTGACAAATTGTGTACAAACACTAAAAAAAACTTGTTTAAA | |
| | TTGTATTTGCAATAAACAACATCAAATTTTTTCATGAAATCTTGGTACAAATTCAGAT | |
| | CTCTTATTTAAAATTTAAATAAGGAATACATTTTCAAAATGCAGTAATCAAAATGTGA | |
| | TCTAGTGTAATGAAATAAAATGTGATCTAGTGTAATGGAAGACCTTTGAGAACCTGGG | |
| | TGTATTAACTTTGTGTATATAGTGTAAATATCCCCACTGTACTGTTAGAGGCCAACAA | |
| | TTCTAGTATGGCTTGTTGGCAAAGAGTGCTACACCGTTTCAATGAAACAATGTATGTT | |
| | TGTTTTAACTGAACTAAAATAAATACATGCTTAATCCTG | |
| | ORF Start ATG at 352 | ORF Stop TAA at 1870 |
| | SEQ ID NO:20 | 506 aa MW at 56025.2 kD |

| | |
|---|---|
| NOV7a, | MKKAEMGRFSISRDEDSSSYSSNSDFNYSYPTKQAALKSHYADVDPENQNFLLESNLG |
| CG120123-02 | KKKYETEFHPGTTSFGMSVFNLSNAIVGSGILGLSYAMANTGIALFIILLTFVSIFSL |
| Protein | YSVHLLLKTANEGGSLLYEQLGYKAFGLVGKLAASGSITMQNIGAMSSYLFIVKYELP |
| Sequence | LVIQALTNIEDKTGLWYLNGNYLVLLVSLVVILPLSLFRNLGYLGYTSGLSLLCMVFF |
| | LIVVICKKFQVPCRVEAALIINETINTTLTQRTALVPALSHNVTENDSCRPHYFIFNS |
| | QTVYAVPILIFSFVCHPAVLRIYEELKDRSRRRMMNVSKISPFAMFLMYLLAALFGYL |
| | TFYEHVESELLHTYSSILGTDILLLIVRLAVLMAVTLTVPVVIFRIRSSVTHLLCASK |
| | DFSWWRHSLITVSILAFTNLLVIFVPTIRDIFGFIGASAASMLIFILPSAFYIKLVKK |
| | FPMKSVQKIGALFFLLSGVLVMTGSMALIVLDWVHNAPGGGH |

Further analysis of the NOV7a protein yielded the following properties shown in Table 7B.

TABLE 7B
Protein Sequence Properties NOV7a

| | |
|---|---|
| PSort | 0.6000 probability located in plasma membrane; 0.4000 |
| analysis: | probability located in Golgi body; 0.3000 probability located |
| | in endosplasmic reticulum (membrane); 0.0300 probability |
| | located in mitochondrial inner membrane |
| SignalP | No Known Signal Sequence Predicted |
| analysis: | |

A search of the NOV7a protein against the Geneseq database, a proprietary database that contains sequences published in patents and patent publication, yielded several homologous proteins shown in Table 7C.

TABLE 7C
Geneseq Results for NOV7a

| | | | Identities/ | |
|---|---|---|---|---|
| | | | Similari- | |
| | | NOV7a | ties | |
| | Protein/ | Residues/ | for the | |
| Geneseq | Organism/Length | Match | Matched | Expect |
| Identifier | [Patent #, Date] | Residues | Region | Value |
| AAE21174 | Human TRICH-18 | 1 . . . 506 | 506/506 | 0.0 |
| | protein - Homo | 1 . . . 506 | (100%) | |
| | sapiens, 506 aa. | | 506/506 | |
| | [WO200212340-A2, | | (100%) | |
| | 14 FEB. 2002] | | | |
| AAB93237 | Human protein | 105 . . . 506 | 400/402 | 0.0 |
| | sequence SEQ ID | 5 . . . 406 | (99%) | |
| | NO: 12239 - Homo | | 401/402 | |
| | sapiens, 406 aa. | | (99%) | |
| | [EP1074617-A2, | | | |
| | 07 FEB. 2001] | | | |
| AAG73492 | Human gene | 190 . . . 506 | 317/317 | e−180 |
| | 26-encoded secreted | 1 . . . 317 | (100%) | |
| | protein fragment, | | 317/317 | |
| | SEQ ID NO: 268 - | | (100%) | |
| | Homo sapiens, 317 aa. | | | |
| | [WO200134628-A1, | | | |
| | 17 MAY 2001] | | | |
| AAE03133 | Human gene 5 | 190 . . . 506 | 317/317 | e−180 |
| | encoded secreted | 1 . . . 317 | (100%) | |
| | protein fragment, | | 317/317 | |
| | SEQ ID NO: 170 - | | (100%) | |
| | Homo sapiens, 317 aa. | | | |
| | [WO200132676-A1, | | | |
| | 10 MAY 2001] | | | |
| AAE16782 | Human transporter | 39 . . . 506 | 288/471 | e−160 |
| | and ion channel-19 | 13 . . . 473 | (61%) | |
| | (TRICH-19) protein - | | 349/471 | |
| | Homo sapiens, 474 aa. | | (73%) | |
| | [WO2001992304-A2, | | | |
| | 06 DEC. 2001] | | | |

In a BLAST searched of public sequence databases, the NOV7a protein w,as fluid to have homology to the proteins shown in the BLASTP data in Fable 7D.

TABLE 7D
Public BLASTP Results for NOV7a

| | | | Identities/ | |
|---|---|---|---|---|
| | | NOV7a | Similarities | |
| Protein | | Residues/ | for the | |
| Accession | Protein/ | Match | Matched | Expect |
| Number | Organism/Length | Residues | Portion | Value |
| Q9HAV3 | Amino acid | 1 . . . 506 | 506/506 (100%) | 0.0 |
| | transporter system | 1 . . . 506 | 506/506 (100%) | |
| | A (Amino acid | | | |
| | transporter system | | | |
| | A2) (KIAA1382 | | | |
| | protein) - | | | |
| | Homo sapiens | | | |
| | (Human), 506 aa. | | | |
| Q96QD8 | Putative 40-9-1 | 1 . . .506 | 505/506 (99%) | 0.0 |
| | protein - Homo | 1 . . . 506 | 506/506 (99%) | |
| | sapiens (Human), | | | |
| | 506 aa. | | | |
| Q9JHE5 | Amino acid | 1 . . . 506 | 448/506 (88%) | 0.0 |
| | system A | 1 . . . 504 | 475/506 (93%) | |
| | transporter | | | |
| | (System A | | | |
| | transporter | | | |
| | isoform 2) - | | | |
| | Rattus norvegicus | | | |
| | (Rat), 504 aa. | | | |
| Q9J188 | Amino acid | 1 . . . 506 | 445/506 (87%) | 0.0 |
| | transporter | 1 . . . 504 | 474/506 (92%) | |
| | system A - | | | |
| | Rattus norvegicus | | | |
| | (Rat), 504 aa. | | | |
| Q9NVA8 | CDNA FLJ10838 | 105 . . . 506 | 400/402 (99%) | 0.0 |
| | fis, clone | 5 . . . 406 | 401/402 (99%) | |
| | NT2RP4001274, | | | |
| | weakly similar | | | |
| | to human | | | |
| | transporter | | | |
| | protein (G17) | | | |
| | mRNA - Homo | | | |
| | sapiens (Human), | | | |
| | 406 aa. | | | |

PFam analysis predicts that the NOV7a protein contains the domains shown in the Table 7E.

TABLE 7E
Domain Analysis of NOV7a

| | NOV7a | Identities/Similarities | Expect |
|---|---|---|---|
| Pfam Domain | Match Region | for the Matched Region | Value |
| Aa_ trans | 95 . . . 489 | 98/476 (21%) | 4.6e−54 |
| | | 298/476 (63%) | |

### Example 8

The NOV8 clone was analyzed, and the nucleotide and encoded polypeptide sequences are shown in Table 8A.

TABLE 8A
NOV8 Sequence Analysis

| | SEQ ID NO:21 | 490 bp |
|---|---|---|
| NOV8a, | CGCCACCATGCCGCCCTACACCGTGGTCTATTTCCCAGTTCGAGGCCGCTGCGCGGCC | |
| CG120814-01 | CTGCGCATGCTGCTGGCAGATCAGGGCCAGAGATGGAAGGAGGAGGTGGTGACCGTGC | |
| DNA | AGACGTGGCAGGAGGGCTCACTCAAAGCCTCCTGCCTATACGGGCAGCTCCCCAAGTT | |
| Sequence | CAAGGCAAGACCTTCATTGTGGGAGACCAGATCTCCTTCGCTGACTACAACCTGCTGG | |
| | ACTTGCTGCTGATCCATGAGGTCCTAGCCCCTGGCTGCCTGGATGCGTTCCCCCTGCT | |
| | CTCAGCATATGTGGGGCGCCTCAGTGCCCGGCCCAAGCTCAAGGCCTTCCTGGCCTCC | |
| | CCTGAGTACGTGAACCTCCCCATCAATGGCAACGGGAAACAGTGAGGGTTGGGGGGAC | |
| | TCTGAGCGGGAGGCAGAGTTTGCCTTCCTTTCTCCAGGACCAATAAAATTTCTAAGAG | |
| | ORF Start: ATG at 8 | ORF Stop: TGA at 242 |
| | SEQ ID NO:22 | 78 aa MW at 8958.4 kD |

| | |
|---|---|
| NOV8a, | MPPYTVVYFPVRGRCAALRMLLADQGQRWKEEVVTVETWQEGSLKASCLYCQLPKFKA |
| CG120814-01 | RPSLWETRSPSLTTTCWTCC |
| Protein | |
| Sequence | |

Further analysis of the NOV8a protein yielded the following properties shown in Table 8B.

TABLE 8B
Protein Sequence Properties NOV8a

| | |
|---|---|
| PSort | 0.7838 probability located in mitochondrial intermembrane |
| analysis: | space; 0.5486 probability located in microbody (peroxisome); |
| | 0.4465 probability located in mitochondrial matrix space; |
| | 0.1352 probability located in mitochondrial inner membrane |
| SignalP | Cleavage site between residues 17 and 18 |
| analysis: | |

A search of the NOV8a protein against the Geneseq database, a proprietary database that contains sequences published in patents and patent publication, yielded several homologous proteins shown in Table 8C.

TABLE 8C
Geneseq Results for NOV8a

| | | | Identities/ | |
|---|---|---|---|---|
| | | NOV8a | Similarities | |
| | Protein/ | Residues/ | for the | |
| Geneseq | Organism/Length | Match | Matched | Expect |
| Identifier | [Patent #, Date] | Residues | Region | Value |
| AAG02025 | Human secreted | 1 . . . 57 | 55/57 (96%) | 2e−27 |
| | protein, SEQ | 1 . . . 57 | 56/57 (97%) | |
| | ID NO: 6106 - | | | |
| | Homo sapiens, 126 aa. | | | |
| | [EP1033401-A2, | | | |
| | 06 SEP. 2000] | | | |
| AAW49014 | Human glutathione | 1 . . . 57 | 55/57 (96%) | 2e−27 |
| | S-transferase | 1 . . . 57 | 56/57 (97%) | |
| | GSTP1c variant - | | | |
| | Homo sapiens, 210 aa. | | | |
| | [WO9821359-A1, | | | |
| | 22 MAY 1998] | | | |
| AAW49013 | Human glutathione | 1 . . . 57 | 55/57 (96%) | 2e−27 |
| | S-transferase | 1 . . . 57 | 56/57 (97%) | |
| | GSTP1b variant - | | | |
| | Homo sapiens, 210 aa. | | | |
| | [WO9821359-A1, | | | |
| | 22 MAY 1998] | | | |
| AAW49012 | Human glutathione | 1 . . . 57 | 55/57 (96%) | 2e−27 |
| | S-transferase | 1 . . . 57 | 56/57 (97%) | |
| | GSTP1a - | | | |
| | Homo sapiens, 210 aa | | | |
| | [WO9821359-A1, | | | |
| | 22 MAY 1998] | | | |
| AAR05448 | Human GSH | 1 . . . 57 | 53/57 (92%) | 2e−24 |
| | transferase - Homo | 1 . . . 56 | 54/57 (93%) | |
| | sapiens, 208 aa. | | | |
| | [WO9001548-A, | | | |
| | 22 FEB. 1990] | | | |

In a BLAST search of public sequence databases, the NOV8a protein was found to have homology to the proteins shown in the BLASTP data in Table 8D.

TABLE 8D
Public BLASTP Results for NOV8a

| | | | Identities/ | |
|---|---|---|---|---|
| | | NOV8a | Similarities | |
| Protein | | Residues/ | for | |
| Accession | Protein/ | Match | the Matched | Expect |
| Number | Organism/Length | Residues | Portion | Value |
| A37378 | glutathione transferase | 1 . . . 57 | 55/57 (96%) | 4e−27 |
| | (EC 2.5.1.18) pi | 1 . . . 57 | 56/57 (97%) | |
| | [validated] - | | | |
| | human, 210 aa. | | | |
| E967676 | SYNTHETIC AMINO | 1 . . . 57 | 55/57 (96%) | 4e−27 |
| | ACID SEQUENCE | 1 . . . 57 | 56/57 (97%) | |
| | FROM THE HUMAN | | | |
| | GSH TRANSFERASE | | | |
| | PI GENE vectors, | | | |
| | 210 aa. | | | |
| CAA00533 | HUMAN GSH | 1 . . . 57 | 55/57 (96%) | 4e−27 |
| | TRANSFERASE P1 | 1 . . . 57 | 56/57 (97%) | |
| | GENE PROTEIN - | | | |
| | synthetic construct, | | | |
| | 210 aa. | | | |
| Q15690 | Glutathione | 1 . . . 57 | 55/57 (96%) | 4e−27 |
| | S-transferase-PIC - | 1 . . . 57 | 56/57 (97%) | |
| | Homo sapiens | | | |
| | (Human), 210 aa. | | | |
| O00460 | Glutathione | 1 . . . 57 | 55/57 (96%) | 4e−27 |
| | S-transferase | 1 . . . 57 | 56/57 (97%) | |
| | (Glutathione | | | |
| | S-transferase pi) - | | | |
| | Homo sapiens | | | |
| | (Human), 210 aa. | | | |

PFam analysis predicts that the NOV8a protein contains the domains shown in the Table 8E.

TABLE 8E
Domain Analysis of NOV8a

| | NOV8a | Identities/Similarities | Expect |
|---|---|---|---|
| Pfam Domain | Match Region | for the Matched Region | Value |
| GST_N | 3 . . . 67 | 16/80 (20%) | 2.6e−06 |
| | | 46/80 (38%) | |

### Example 9

The NOV9 clone was analyzed, and the nucleotide and encoded polypeptide sequences are shown in Table 9A.

TABLE 9A
NOV9 Sequence Analysis

| | SEQ ID NO:23 | 625 bp |
|---|---|---|
| NOV9a, | TCATGGCCTCCGGTAACATGCACATTGGAAAGCTCACCCCTGACTTCAAGGCCACTGC | |
| CG122768-01 | CGTGGTGGATGGCACCTACAGGGAGGTAAAGCTGTTGGACTACAGAGGGAAGCACGTG | |
| DNA | GTCCTCTTTTTCCATCCTCTGGACTTCACTTTTTTTTTTCCCACAGAGATCATCGCAT | |
| Sequence | TCAGCGACCATGCTGAGGGCTTCCGAAGCTGCAAAGTTGCAAAGTGCTGGGGACCTC | |
| | GGTGGGCTCACAGTTCACCCACCTGGCTTGGATCAACATCCCCCGGAAGGAGGGAGGC | |
| | TTTGAGTCCCTGGACACCCCTCTGCTTGCTGACGTGACCCTGAAGTTGTCTGAGAATT | |
| | ACGGCGTGTTGAAAACAGACGAGGGCATTGTCTGCAGGGGCCTCTTTATCATCCATGG | |
| | CAAGGATGTCCTTCCCCAGATCGCTGTTAATGATTGGCCTGTGGGACACTTTGTGGAT | |
| | GAGGCCCTGCGGCTGGTCCAGGCCTTCCAGTACACAGACGAGCACCCGGAAATTTGTC | |
| | CTGCTGGCTGGAAGCCTGGCAGTGACATGATCAAGCCCAGCGTGAATGACAGCAAGGA | |
| | ATATTTCTCCAAACACAACTAGGCTGGCTGATGGATCATGAGCTT | |
| | ORF Start: ATG at 3 | ORF Stop: TAG at 600 |
| | SEQ ID NO:24 | 199 aa MW at 22326.3 kD |

| | |
|---|---|
| NOV9a, | MASGNMHIGKLTPDFKATAVVDGTYREVKLLDYRGKHVVLFFHRLDFTFFFPTEIIAF |
| CG122768-01 | SDHAEGFRKLQSCKVLGTSVGSQFTHLAWTNIPRKEGGFESLDTPLLADVTLKLSENY |
| Protein | IGVLKTDEGIVCRGLFIIHGKDVLPQIAVNDWPVGHFVDEALRLVQAFQYTDEHREICP |
| Sequence | AGWKPGSDMIKPSVNDSKEYFSKHN |

Further analysis of the NOV9a protein yielded the following properties shown in Table 9B.

TABLE 9B
Protein Sequence Properties NOV9a

| | |
|---|---|
| PSort | 0.6400 probability located in microbody (peroxisome); 0.4500 |
| analysis: | probability located in cytoplasm; 0.1569 probability located |
| | in lysosome (lumen); 0.1000 probability located in |
| | mitochondrial matrix space |
| SignalP | No Known Signal Sequence Predicted |
| analysis: | |

A search of the NOV9a protein against the Geneseq database, a proprietary database that contains sequences published in patents and patent publication, yielded several homologous proteins shown in Table 9C.

TABLE 9C
Geneseq Results for NOV9a

| | | | Identities/ | |
|---|---|---|---|---|
| | | NOV8a | Similarities | |
| | Protein/ | Residues/ | for the | |
| Geneseq | Organism/Length | Match | Matched | Expect |
| Identifier | [Patent #, Date] | Residues | Region | Value |
| AAU78580 | Mouse | 1 . . . 199 | 155/199 (77%) | 1e−85 |
| | peroxiredoxin | 1 . . . 198 | 166/199 (82%) | |
| | II-1 (Prxll-1) | | | |
| | protein - | | | |
| | Mus sp, 198 aa. | | | |
| | [KR99066020-A, | | | |
| | 16 AUG. 1999] | | | |
| AAB68036 | Amino acid | 1 . . . 199 | 155/199 (77%) | 9e−85 |
| | sequence of the | 1 . . . 198 | 168/199 (83%) | |
| | acid form of | | | |
| | peroxyredoxin | | | |
| | TDX1 - Homo | | | |
| | sapiens, 198 aa. | | | |
| | [FR2798672-A1, | | | |
| | 23 MAR. 2001] | | | |
| ABG26215 | Novel human | 22 . . . 199 | 136/178 (76%) | 4e−74 |
| | diagnostic protein | 43 . . . 219 | 150/178 (83%) | |
| | #26206 - Homo | | | |
| | sapiens, 219 aa. | | | |
| | [W0200175067- | | | |
| | A2, | | | |
| | 11 OCT. 2001] | | | |
| ABG26215 | Novel human | 22 . . . 199 | 136/178 (76%) | 4e−74 |
| | diagnostic protein | 43 . . . 219 | 150/178 (83%) | |
| | #26206 - Homo | | | |
| | sapiens, 219 aa. | | | |
| | [WO200175067- | | | |
| | A2, | | | |
| | 11 OCT. 2001] | | | |
| AAW09794 | Natural killer | 1 . . . 199 | 138/199 (69%) | 2e−70 |
| | cell enhancing | 1 . . . 178 | 151/199 (75%) | |
| | factor B - | | | |
| | Homo sapiens, | | | |
| | 178 aa. | | | |
| | [U.S. Pat. No. | | | |
| | 5610286-A, | | | |
| | 11 MAR. 1997] | | | |

In a BLASTP search of public sequence databases, the NOV9a protein was found to have homology to the proteins shown in the BLASTP data in Table 9D.

TABLE 9D
Public BLASTP Results for NOV9a

| | | | Identities/ | |
|---|---|---|---|---|
| | | NOV9a | Similarities | |
| Protein | | Residues/ | for | |
| Accession | Protein/ | Match | the Matched | Expect |
| Number | Organism/Length | Residues | Portion | Value |
| P35704 | Peroxiredoxin 2 | 1 . . . 199 | 154/199 (77%) | 1e−84 |
| | (Thioredoxin | 1 . . . 198 | 165/199 (82%) | |
| | peroxidase 1) | | | |
| | (Thioredoxin- | | | |
| | dependent peroxide | | | |
| | reductase 1) | | | |
| | (Thiol-specific | | | |
| | antioxidant protein) | | | |
| | (TSA) - | | | |
| | Rattus norvegicus | | | |
| | (Rat), 198 aa. | | | |
| P32119 | Peroxiredoxin 2 | 1 . . . 199 | 155/199 (77%) | 2e−84 |
| | (Thioredoxin | 1 . . . 198 | 168/199 (83%) | |
| | peroxidase 1) | | | |
| | (Thioredoxin- | | | |
| | dependent peroxide | | | |
| | reductase | | | |
| | (TSA) (PRP) | | | |
| | (Natural killer | | | |
| | cell enhancing | | | |
| | factor B) | | | |
| | (NKEF-B) - | | | |
| | Homo sapiens | | | |
| | (Human), 198 aa. | | | |
| Q61171 | Peroxiredoxin 2 | 1 . . . 199 | 154/199 (77%) | 3e−84 |
| | (Thioredoxin | 1 . . . 198 | 165/199 (82%) | |
| | peroxidase 1) | | | |
| | (Thioredoxin- | | | |
| | dependent peroxide | | | |
| | reductase 1) | | | |
| | (Thiol-specific | | | |
| | antioxidant protein) | | | |
| | (TSA) - Mus | | | |
| | musculus (Mouse), | | | |
| | 198 aa. | | | |
| O88376 | Type II | 1 . . . 199 | 154/199 (77%) | 4e−84 |
| | peroxiredoxin 1 - | 1 . . . 198 | 165/199 (82%) | |
| | Mus musculus | | | |
| | (Mouse), 198 aa. | | | |
| Q9CWJ4 | Peroxiredoxin 2 - | 1 . . . 199 | 153/199 (76%) | 6e−84 |
| | Mus musculus | 1 . . . 198 | 165/199 (82%) | |
| | (Mouse), 198 aa. | | | |

PFam analysis predicts that the NOV9a protein contains the domains shown in the Table 9E.

TABLE 9E
Domain Analysis of NOV9a

| | NOV9a | Identities/Similarities | Expect |
|---|---|---|---|
| Pfam Domain | Match Region | for the Matched Region | Value |
| AhpC-TSA | 8 . . . 158 | 78/162 (48%) | 2.1e−49 |
| | | 121/162 (75%) | |

### Example 10

The NOV10 clone as analyzed, and the nucleotide and encoded polypeptide sequences are shown in Table 10A.

TABLE 10A
NOV10 Sequence Analysis

| | SEQ ID NO:25 | 1081 bp |
|---|---|---|
| NOV10a. | CTGGGGATGATGACGGATCTGAAGCAAAGCCATTCAGTGAGGCTGAATGATGGACCCT | |
| CG122786-01 | TCATGCCAGTGCTGGGATTTGGCACTTATGCTCCTGATCATGTAAGTGGACCCCAGGA | |
| DNA | GGCTGAAGTTTCTCCCAAAAGCCAGGCTGCCGAGGCCACCAAAGTGGCTATTGACGTA | |
| Sequence | GGCTTCCGCCATATTGATTCAGCATACTTATACCAAAATGAGGAGGAGGTTGGACAGG | |
| | CCATTTGGGAGAAGATCGCTGATGGTACCGTCAAGAGAGAGGAAATATTCTACACCAT | |
| | CAAGCTTTGGGCTACTTTCTTTCGGGCAGAATTGGTTCACCCGGCCCTAGAAAGGTCA | |
| | CTGAAGAAACTTGGACCGGACTATGTAGATCTCTTCATTATTCATGTACCATTTGCTA | |
| | TGAAGTTCTTTATCTTCTTTTCTATTTTCCAGCCTGGGAAAGAATTACTGCCAAAGGA | |
| | TGCCAGTGGAGAGATTATTTTAGAAACTGTGGAGCTTTGTGACACTTGGGAGGTACAG | |
| | GCCCTGGAGAAGTGCAAAGAAGCAGGTTTAACCAGGTCCATTGGGGTGTCCAATTTCA | |
| | ATCACAAGCTGCTGGAACTCATCCTCAACAAGCCAGGGCTCAAGTACAAGCCCACCTG | |
| | CAACCAGGTGCAGGTGGAATGTCACCCTTACCTCAACCAGAGCAAACTCCTGGAGTTC | |
| | TGCAAGTCCAAGGACATTGTTCTAGTTGCCTACAGTGCCCTGGGATCCCAAAGAGACC | |
| | CACAGTGGGTGGATCCCGACTGCCCACATCTCTTGGAGGAGCCGATCTTGAAATCCAT | |
| | TGCCAAGAAACACAGTGGAAGCCCAGGCCAGGTCGCCCTGCGCTACCAGCTGCAGCGG | |
| | GGAGTGGTGGTGCTGGCCAAGAGCTTCTCTCAGGAGAGAATCAAAGAGAACTTCCAGG | |
| | TATCCTTTCAGATTTTTGACTTTGAGTTGACTCCAGAGGACATGAAAGCCATTGATGG | |
| | CCTCAACAGAAATCTCCGATATGACAAGTTACAATTGGCTAATCACCCTTATTTTCCA | |
| | TTTTCTGAAGAATATTGACCATGAGCTATTGAACATT | |
| | ORF Start: ATG at 7 | ORF Stop: TGA at 1060 |
| | SEQ ID NO:26 | 351 aa MW at 40003.5 kD |

| | |
|---|---|
| NOV10a, | MMTDLKQSHSVRLNDGPFMPVLGFGTYAPDHVSGPQEAEVSPKSQAAEATKVAIDVGF |
| CG122786-01 | AARHIDSAYLYQNEEEVGQAIWEKIADGTVKREEIFYTTKLWATFFRAELVHPALERSLK |
| Protein | KLGPDYVDLFIIHVPFANKFFIFFSIFQPGKELLPKDASGEIILETVELCDTWEVQAL |
| Sequence | EKCKEAGLTRSIGVSNFNHKLLELILNKPGLKYKPTCNQVQVECHPYLNQSKLLEFCK |
| | SKDIVLVAYSALGSQRDPQWAAVDPDCPHLLEEPILKSIAKKHSGSPGQVALRYQLQRGV |
| | VVLAKSFSQERIKENFQVSFQIFDFELTPEDMKAIDGLNPAANLRYDKLQLANHPYPRFS |
| | EEY |

Further analysis of the NOV10a protein yielded the following, properties shown in Table 10B.

TABLE 10B
Protein Sequence Properties NOV10a

| | |
|---|---|
| PSort | 0.7000 probability located in plasma membrane; 0.5312 |
| analysis: | probability located in microbody (peroxisome); 0.2000 |
| | probability located in endoplasmic reticulum (membrane); |
| | 0.1000 probability located in mitochondrial inner membrane |
| SignalP | No Known Signal Sequence Predicted |
| analysis: | |

A search of the NOV10a protein against the Geneseq database, a proprietary database that contains sequences published in patents and patent publication, yielded several homologous proteins shown in Table 10C.

TABLE 10C
Geneseq Results for NOV10a

| | | | Identities/ | |
|---|---|---|---|---|
| | | NOV10a | Similarities | |
| | Protein/ | Residues/ | for the | |
| Geneseq | Organism/Length | Match | Matched | Expect |
| Identifier | [Patent #, Date] | Residues | Region | Value |
| ABB07529 | Human drug | 11 . . . 351 | 287/342 (83%) | e−157 |
| | metabolizing | 8 . . . 323 | 297/342 (85%) | |
| | enzyme (DME) | | | |
| | (ID: | | | |
| | 7478994CDI) - | | | |
| | Homo sapiens, | | | |
| | 323 aa. | | | |
| | [WO200204612- | | | |
| | A2, | | | |
| | 17 JAN. 2002] | | | |
| AAM79455 | Human protein | 4 . . . 351 | 222/349 (63%) | e−121 |
| | SEQ ID NO | 4 . . . 325 | 271/349 (77%) | |
| | 3101 - | | | |
| | Homo sapiens, | | | |
| | 325 aa. | | | |
| | [WO200157190- | | | |
| | A2, | | | |
| | 09 AUG. 2001] | | | |
| AAM78471 | Human protein | 4 . . . 351 | 222/349 (63%) | e−121 |
| | SEQ ID NO | 2 . . . 323 | 271/349 (77%) | |
| | 1133 - | | | |
| | Homo sapiens, | | | |
| | 323 aa. | | | |
| | [WO200157190- | | | |
| | A2, | | | |
| | 09 AUG. 2001] | | | |
| AAW14799 | Type 5 17-beta- | 4 . . . 351 | 222/349 (63%) | e−121 |
| | hydroxysteroid | 2 . . . 323 | 271/349 (77%) | |
| | dehydrogenase - | | | |
| | Homo sapiens, | | | |
| | 323 aa. | | | |
| | [WO9711162- | | | |
| | A1, | | | |
| | 27 MAR. 1997] | | | |
| AAB43444 | Human cancer | 1 . . . 351 | 218/353 (61%) | e−118 |
| | associated protein | 10 . . . 336 | 270/353 (75%) | |
| | sequence SEQ ID | | | |
| | NO: 889 - Homo | | | |
| | 21 SEP. 2000] | | | |

In a BLAST search of public sequence databases, the NOV10a protein was found to have homology to the proteins shown in the BLASTP data in Table 10D.

TABLE 10D
Public BLASTP Results for NOV10a

| | | | Identities/ | |
|---|---|---|---|---|
| | | NOV10a | Similarities | |
| Protein | | Residues/ | for | |
| Accession | Protein/ | Match | the Matched | Expect |
| Number | Organism/Length | Residues | Portion | Value |
| P05980 | Prostaglandin-F | 7 . . . 351 | 231/346 (66%) | e−124 |
| | synthase 1 | 4 . . . 323 | 271/346 (77%) | |
| | (EC 1.1.1.188) | | | |
| | (PGF synthase 1) | | | |
| | (PGF 1) | | | |
| | (Prostaglandin-D2 | | | |
| | 11 reductase 1) | | | |
| | (PGFS1) - | | | |
| | Bos taurus | | | |
| | (Bovine), 323 aa. | | | |
| P52897 | Prostaglandin-F | 7 . . . 351 | 231/346 (66%) | e−124 |
| | synthase 2 | 4 . . . 323 | 270/346 (77%) | |
| | (EC 1.1.1.188) | | | |
| | (PGF synthase 2) | | | |
| | (PGF 2) | | | |
| | (Prostaglandin-D2 | | | |
| | 11 reductase 2) | | | |
| | (PGFSII) - | | | |
| | Bos taurus | | | |
| | (Bovine). 323 aa. | | | |
| P52898 | Dihydrodiol | 11 . . . 351 | 229/342 (66%) | e−123 |
| | dehydrogenase 3 | 8 . . . 323 | 266/342 (76%) | |
| | (EC 1.-.-.-) | | | |
| | (Prostaglandin F | | | |
| | synthase) - | | | |
| | Bos taurus | | | |
| | (Bovine), 323 aa. | | | |
| P42330 | Aldo-keto | 4 . . . 351 | 222/349 (63%) | e−121 |
| | reductase family 1 | 2 . . . 323 | 271/349 (77%) | |
| | member C3 | | | |
| | (EC 1.1.1.-) | | | |
| | (Trans-1,2- | | | |
| | dihydrobenzene- | | | |
| | 1,2-diol | | | |
| | dehydrogenase) | | | |
| | (EC 1.3.1.20) | | | |
| | (Chlordecone | | | |
| | reductase homolog | | | |
| | HAKRb) | | | |
| | (HA1753) | | | |
| | (Dihydrodiol | | | |
| | dehydrogenase, | | | |
| | type 1) | | | |
| | (Dihydrodiol | | | |
| | dehydrogenase 3) | | | |
| | (DD3) (3- | | | |
| | alpha- | | | |
| | hydroxysteroid | | | |
| | dehydrogenase) | | | |
| | (3alpha-HSD) | | | |
| | (Prostaglandin F | | | |
| | synthase) | | | |
| | (EC 1.1.1.188) - | | | |
| | Homo sapiens | | | |
| | (Human), 323 aa. | | | |
| B57407 | 3alpha- | 4 . . . 351 | 222/349 (63%) | e−120 |
| | hydroxysteroid | 2 . . . 323 | 270/349 (76%) | |
| | dehydrogenase | | | |
| | (EC 1.1.1.-) II - | | | |
| | human, 323 aa. | | | |

PFam analysis predicts that the NOV10a protein contains the domains shown in the Table 10E.

TABLE 10E
Domain Analysis of NOV10a

| | NOV10a | Identities/Similarities | Expect |
|---|---|---|---|
| Pfam Domain | Match Region | for the Matched Region | Value |
| aldo_ket_red | 13 . . . 332 | 162/383 (42%) | 7.1e−124 |
| | | 269/383 (70%) | |

### Example 11

The NOV11 clone was analyzed, and the nucleotide and encoded polypeptide sequences are shown in Table 11A.

TABLE 11A
NOV11 Sequence Analysis

| | SEQ ID NO:27 | 698 bp |
|---|---|---|
| NOV11a, | AAAAAAATCTGCATGAGCATGTATCCACCCATTAATGGCCTTGACTGGGCCAATATTTh | |
| CG122795-01 | TTGTCGTCGGCGGATCTGCATGGGGTGGGTGTTCCACGCTGTTAATGAATGAACTCGA | |
| DNA | AAGGGTTTCGTTCGACCTGGCGTGTAATTTGCTGATTTGGGTGGGAGACCTTGTTGCC | |
| Sequence | CGCGGCGCGAAAAACGTCGAGTGCCTGAACTTGATTACTATGCCTTGGTTCCGGGCTG | |
| | TGCGAGGTAACCATGAGCAGATGATGATTGATGGGCTATCGGAGTATGGAAACGTTAA | |
| | CCACTGGCTGGAAAACGGCGGCGTGTGGTTCTTCAGTCTTGATTATGAAAAAGAGGTG | |
| | CTGGCTAAGGCTCTGGTTCATAAATCGGCCAGCCTGCCATTCGTCATCGAGCTGGTTA | |
| | CCGCTGAACGTAAAATCGTTATCTGCCACGCTGACTACCCGCATAACGAATATGCGTT | |
| | CGACAAGCCGGTCCCGAAAGACATGGTCATCTGGAATCGTGAACGGGTTAGCGACGCT | |
| | CAGGACGGCATTGTCTCGCCGATAGCTGGTGCTGATCTGTTTATCTTCGGCCACACCC | |
| | CTGCGCGCCAGCCCCTGAAGTATGCCAACCAGATGTACATCGATACTGGTGCCGTGTT | |
| | CTGCGGAAACCTCACGCTGGTACAGGTTCAAGGTGGTGCCCATGCGTAAACCATCCCG | |
| | CC | |
| | ORF Start: ATG at 13 | ORF Stop: TAA at 685 |
| | SEQ ID NO:28 | 224 aa MW at 24915.4 kD |

| | |
|---|---|
| NOV11a. | MSMYPPINGLDWANIFVVGGSAWGGCSTLLMNELERVSFDLACNLLIWVGDLVARGAK |
| CG122795-01 | NVECLNLITMPWFRAVRGNHEQMMIDGLSFYGNVNHWLENGGVWFFSLDYEKEVLAKA |
| Protein | LVHKSASLPFVIELVTAERKIVICHADYPHNEYAFDKRVPKDMVIWNRERVSDAQDGI |
| Sequence | VSPIAGADLFIFGHTPARQPLKYANQMYIDTGAVFCGNLTLVQVQGGAHA |

Further analysis of the NOV11a protein yielded the following properties shown in Table 11B.

TABLE 11B
Protein Sequence Properties NOV11a

| | |
|---|---|
| PSort | 0.5500 probability located in endoplasmic reticulum |
| analysis | (membrane); 0.3479 probability located in lysosome (lumen); |
| | 0.2518 probability located in microbody (peroxisome); 0.1000 |
| | probability located in endoplasmic reticulum (lumen) |
| SignalP | No Known Signal Sequence Predicted |
| analysis: | |

A search of the NOV11a protein against the Geneseq database, a proprietary database that contains sequences published in patents and patent publication, yielded several homologous proteins shown in Table 11C.

TABLE 11C
Geneseq Results for NOV11a

| | | | Identities/ | |
|---|---|---|---|---|
| | | NOV11a | Similarities | |
| | Protein/ | Residues/ | for the | |
| Geneseq | Organism/Length | Match | Matched | Expect |
| Identifier | [Patent #, Date] | Residues | Region | Value |
| ABG01589 | Novel human | 36 . . . 220 | 175/185 (94%) | e−101 |
| | diagnostic | 58 . . . 242 | 179/185 (96%) | |
| | protein #1580 - | | | |
| | Homo sapiens, | | | |
| | 634 aa. | | | |
| | [WO200175067- | | | |
| | A2, | | | |
| | 11 OCT. 2001] | | | |
| ABG01589 | Novel human | 36 . . . 220 | 175/185 (94%) | e−101 |
| | diagnostic | 58 . . . 242 | 179/185 (96%) | |
| | protein #1580 - | | | |
| | Homo sapiens, | | | |
| | 634, aa. | | | |
| | [WO200175067- | | | |
| | A2, | | | |
| | 11 OCT. 2001] | | | |
| ABG01590 | Novel human | 1 . . . 180 | 163/180 (90%) | 7e−91 |
| | diagnostic | 12 . . . 189 | 166/180 (91%) | |
| | protein #1581 - | | | |
| | Homo sapiens, | | | |
| | 515 aa. | | | |
| | [WO200175067- | | | |
| | A2, | | | |
| | 11 OCT. 2001] | | | |
| ABG01590 | Novel human | 1 . . . 180 | 163/180 (90%) | 7e−91 |
| | diagnostic | 12 . . . 189 | 166/180 (91%) | |
| | protein #1581 - | | | |
| | Homo sapiens, | | | |
| | 515 aa. | | | |
| | [WO200175067- | | | |
| | A2, | | | |
| | 11 OCT. 2001] | | | |
| ABG18236 | Novel human | 9 . . . 130 | 107/122 (87%) | 6e−56 |
| | diagnostic protein | 49 . . . 168 | 110/122 (89%) | |
| | #18227 - | | | |
| | Homo sapiens, | | | |
| | 193 aa. | | | |
| | [WO200175067- | | | |
| | A2, | | | |
| | 11 OCT. 2001] | | | |

In a BLAST search of public sequence databases, the NOV11a protein was found to have homology to the proteins shown in the BLASTP data in Table 11D.

TABLE 11D
Public BLASTP Results for NOV11a

| | | NOV11a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| P03772 | Serine/threonine | 1 . . . 220 | 152/220 (69%) | 5e−85 |
| | protein phosphatase | 1 . . . 218 | 176/220 (79%) | |
| | (EC 3.1.3.16)— | | | |
| | Bacteriophage | | | |
| | lambda, 221 aa. | | | |
| Q8X993 | Hypothetical 25.1 | 1 . . . 220 | 151/220 (68%) | 3e−84 |
| | kDa protein | 1 . . . 218 | 175/220 (78%) | |
| | (Putative serine/ | | | |
| | threonine protein | | | |
| | phosphatase)— | | | |
| | Escherichia coli | | | |
| | O157:H7, 221 aa. | | | |
| Q8X3X2 | Hypothetical protein | 81 . . . 220 | 103/140 (73%) | 1e−57 |
| | z0954—Escherichia | 1 . . . 140 | 118/140 (83%) | |
| | coli O157:H7, | | | |
| | 143 aa. | | | |
| Q8XCL4 | Protein phosphatase | 3 . . . 219 | 95/217 (43%) | 2e−41 |
| | 1 modulates | 8 . . . 219 | 127/217 (57%) | |
| | phosphoproteins, | | | |
| | signals protein | | | |
| | misfolding | | | |
| | (Phosphoprotein | | | |
| | phosphatase 1)— | | | |
| | Escherichia coli | | | |
| | O157:H7, 219 aa. | | | |
| F64945 | Phosphoprotein | 3 . . . 219 | 94/217 (43%) | 1e−40 |
| | phosphatase | 8 . . . 219 | 126/217 (57%) | |
| | (EC 3.1.3.16) 1, | | | |
| | serine/threonine | | | |
| | specific— | | | |
| | Escherichia coli | | | |
| | (strain K-12), | | | |
| | 219 aa. | | | |

PFam analysis predicts that the NOV11a protein contains the domains shown in the Table 11E.

TABLE 11E
Domain Analysis of NOV11a

| | | | |
|---|---|---|---|
| Pfam | NOV11a | Identities/Similarities | Expect |
| Domain | Match Region | for the Matched Region | Value |

No Significant Matches Found

### Example 12

The NOV12 clone was analyzed, and the nucleotide and encoded polypeptide sequences are shown in Table 12A.

TABLE 12A
NOV12 Sequence Analysis

| | SEQ ID NO:29 | 1540 bp |
|---|---|---|
| NOV12a, | CCAGACATGGGACTGGAGGACGAGCAAAAGATGCTTACCGAATCCGGAGATCCTGAGG | |
| CG122805-01 | AGGAGGAAGAGGAAGAGGAGGAATTAGTGATAGGACTGAGGCTTTCAGTGCATACTGG | |
| DNA | CAACCTTGGAAGGCAGGAATGTGAAACTTTTCCCTACTACTTAGCATCAGAATTGAAT | |
| Sequence | AAGGGAGACCGCATTCTGCCATTTCTGGCGGCAGTGTGGCTCTGCCAGCTGGCCTTCTGC | |
| | ACGGATCCCCTAACAACAGTGAGAGAGCAATGCGAGCAAGTTGGAGAAATGTGTAAGG | |
| | CCCGGGAGCGGCTAGAGCTCTGTGATGAGCGTGTATCCTCTCGATCACATACAGAAGA | |
| | GGATTGCACGGAGGAGCTCTTTGACTTCTTGCATGCGAGGGACCATTGCGTGGCCCAC | |
| | AAACTCTTTAACAACTTGAAATAAATGTGTGGACTTAATTCACCCCAGTCTTCATCAT | |
| | CTGGGCATCAGAATATTTCCTTATGGTTTTGGATGTACCATTTGTCTCTTATCTGTGT | |
| | AACTGTAAGTCACATGAA | |
| | ORF Start: ATG at 173 | ORF Stop: TAA at 428 |
| | SEQ ID NO:30 | 85 aa MW at 9953.2 kD |

| | |
|---|---|
| NOV12a, | MGDRILPFLAAVWLCQLAFCTDPLTTVREQCEQLEKCVKARERLELCDERVSSRSHTE |
| CG122805-01 | FDCTEELFDFLHARDHCVAHKLFNNLK |
| Protein | |
| Sequence | |

Further analysis of the NOV12a protein yielded the following properties shown in Table 12B.

TABLE 12B
Protein Sequence Properties NOV12a

| | |
|---|---|
| PSort | 0.6711 probability located in outside: 0.1000 probability |
| analysis: | located in endoplasmic reticulum (membrane); 0.1000 |
| | probability located in endoplasmic reticulum (lumen); |
| | 0.1000 probability located in lysosome (lumen) |
| SignalP | Cleavage site between residues 21 and 22 |
| analysis: | |

A search of the NOV12a protein against the Geneseq database, a proprietary database that contains sequences published in patents and patent publication, yielded several homolgous proteins shown in Table 12C.

TABLE 12C
Geneseq Results for NOV12a

| | | NOV12a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| ABG11307 | Novel human | 22 . . . 85 | 64/64 (100%) | 5e−33 |
| | diagnostic protein | 6 . . . 69 | 64/64 (100%) | |
| | #11298—Homo | | | |
| | sapiens, 69 aa. | | | |
| | [WO200175067-A2, | | | |
| | 11 OCT. 2001] | | | |
| AA013622 | Human polypeptide | 22 . . . 85 | 64/64 (100%) | 5e−33 |
| | SEQ ID NO | 30 . . . 93 | 64/64 (100%) | |
| | 27514—Homo | | | |
| | sapiens, 93 aa. | | | |
| | [WO200164835-A2, | | | |
| | 7 SEP. 2001] | | | |
| ABG11307 | Novel human | 22 . . . 85 | 64/64 (100%) | 5e−33 |
| | diagnostic protein | 6 . . . 69 | 64/64 (100%) | |
| | #11298—Homo | | | |
| | sapiens, 69 aa. | | | |
| | [WO200175067-A2, | | | |
| | 11 OCT. 2001] | | | |
| AA013554 | Human polypeptide | 22 . . . 85 | 57/64 (89%) | 4e−29 |
| | SEQ ID NO | 30 . . . 93 | 62/64 (96%) | |
| | 27446—Homo | | | |
| | sapiens, 93 aa. | | | |
| | [WO200164835-A2, | | | |
| | 7 SEP. 2001] | | | |
| AAO07352 | Human polypeptide | 22 . . . 85 | 53/64 (82%) | 2e−25 |
| | SEQ ID NO | 6 . . . 69 | 57/64 (88%) | |
| | 21244—Homo | | | |
| | sapiens, 75 aa. | | | |
| | [WO200164835-A2, | | | |
| | 7 SEP. 2001] | | | |

In a BLAST search of public sequence databases, the NOV12a protein was found to have homology to the proteins shown in the BLASTP data in Table 12D.

TABLE 12D
Public BLASTP Results for NOV12a

| | | NOV12a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| P07919 | Ubiquinol-cytochrome | 22 . . . 85 | 64/64 (100%) | 1e−32 |
| | C reductase complex | 28 . . . 91 | 64/64 (100%) | |
| | 11 kDa protein, mito- | | | |
| | chondrial precursor | | | |
| | (EC 1 10.2.2) | | | |
| | (Mitochondrial hinge | | | |
| | protein) (Cytochrome | | | |
| | C1, nonheme 11 kDa | | | |
| | protein) (Complex III | | | |
| | subunit VIII)—Homo | | | |
| | sapiens (Human), | | | |
| | 91 aa. | | | |
| S00219 | ubiquinol-cytochrome- | 22 . . . 85 | 63/64 (98%) | 7e−32 |
| | c reductase (EC | 28 . . . 91 | 63/64 (98%) | |
| | 1.10.2.2) 11 K protein | | | |
| | precursor—human, | | | |
| | 91 aa. | | | |
| P00126 | Ubiquinol-cytochrome | 22 . . . 85 | 61/64 (95%) | 2e−30 |
| | C reductase complex | 15 . . . 78 | 62/64 (96%) | |
| | 11 kDa protein | | | |
| | (EC 1.10.2.2) | | | |
| | (Mitochondrial hinge | | | |
| | protein) (Cytochrome | | | |
| | C1, nonheme 11 kDa | | | |
| | protein) (Complex III | | | |
| | subunit VIII)—Bos | | | |
| | taurus (Bovine), 78 aa. | | | |
| Q8SPH5 | Ubiquinol-cytochrome | 22 . . . 85 | 60/64 (93%) | 7e−30 |
| | c reductase hinge | 28 . . . 91 | 62/64 (96%) | |
| | protein—Macaca | | | |
| | fascicularis (Crab | | | |
| | eating macaque) | | | |
| | (Cynomolgus | | | |
| | monkey), 91 aa. | | | |
| P99028 | Ubiquinol-cytochrome | 22 . . . 85 | 60/64 (93%) | 7e−30 |
| | C reductase complex | 26 . . . 89 | 60/64 (93%) | |
| | 11 kDa protein, mito- | | | |
| | chondrial precursor | | | |
| | (EC 1.10.2.2) | | | |
| | (Mitochondrial hinge | | | |
| | protein) (Cytochrome | | | |
| | C1, nonheme 11 kDa | | | |
| | protein) (Complex III | | | |
| | subunit VIII)—Mus | | | |
| | musculus (Mouse), | | | |
| | 89 aa. | | | |

PFam analysis predicts that the NOV12a protein contains the domains shown in the Table 12E.

TABLE 12E
Domain Analysis of NOV12a

| Pfam | NOV12a | Identities/Similarities | Expect |
|---|---|---|---|
| Domain | Match Region | for the Matched Region | Value |
| UCR_hinge | 21 . . . 85 | 50/65 (77%) | 7.2e−44 |
| | | 64/65 (98%) | |

### Example 13

The NOV13 clone was analyzed, and the nucleotide and encoded polypeptide sequences are shown in Table 13A.

TABLE 13A
NOV13 Sequence Analysis

| | SEQ ID NO:31 | 3057 bp |
|---|---|---|
| NOV13a, | CGCGCAGCTGCCCCCATGGCTTTGCGGGGCGCCGCGGGAGCGACCGACACCCCGGTGT | |
| CG123100-01 | CCTCGGCCGGGGGAGCCCCCGGCGGCTCAGCGTCCTCGTCGTCCACCTCCTCGGGCGG | |
| DNA | CTCGGCCTCGGCGGGCGCGGGGCTGTGGGCCGCGCTCTATGACTACGAGGCTCGCGGC | |
| Sequence | GAGGACGAGCTGAGCCTGCGGCGCGGCCAGCTGGTGGAGGTGCTGTCGCAGGACGCCG | |
| | CCGTGTCGGGCGACGAGGGCTGGTGGGCAGGCCAGGTGCAGCGGCGCCTCGGCATCTT | |
| | CCCCCCCAACTACGTGGCTCCCTGCCGCCCGGCCGCCAGCCCCGCGCCGCCGCCCTCG | |
| | CGGCCCAGCTCCCCGGTACACGTCGCCTTCGAGCGGCTGGAGCTGAAGGAGCTCATCG | |
| | GCGCTGGGGGCTTCGGGCAGGTGTACCGCGCCACCTGGCAGGGCCAGGAGGTGGCCGT | |
| | GAAGGCGGCGCGCCAGGACCCGGAGCAGGACGCGGCGGCGGCTGCCGAGAGCGTGCGG | |
| | CGCGAGGCTCGGCTCTTCGCCATGCTGCGGCACCCCAACATCATCGAGCTGCGCGGCG | |
| | TGTGCCTGCAGCAGCCGCACCTCTGCCTGGTGCTGGAGTTCGCCCGCGGCGGAGCGCT | |
| | CAACCGAGCGCTGGCCGCTGCCAACGCCGCCCCGGACCCGCGCGCGCCCGGCCCCCGC | |
| | CGCGCGCGCCGCATCCCTCCGCACGTGCTGGTCAACTGGGCCGTGCAGATAGCGCGGG | |
| | GCATGCTCTACCTGCATGAGGAGGCCTTCGTGCCCATCCTGCACCGGGACCTCAAGTC | |
| | CAGCAACATTTTGCTACTTGAGAAGATAGAACATGATGACATCTGCAATAAAACTTTG | |
| | AAGATTACAGATTTTGGGTTGGCGAGGGAATGGCACAGGACCACCAAAATGAGCACAG | |
| | CAGGCACCTATGCCTGGATGGCCCCCGAAGTGATCAAGTCTTCCTTGTTTTCTAAAGGG | |
| | AAGCGACATCTGGAGCTATGGAGTGCTGCTGTGGGAAAACTGCTCACCGGAGAAGTCCCC | |
| | TATCGGGGCATTGATGGCCTCGCCGTGGCTTATGGGGTAGCAGTCAATAAACTCACTT | |
| | TGCCCATTCCATCCACCTGCCCTGAGCCGTTTGCCAAGCTCATGAAAGAATGCTGGCA | |
| | ACAAGACCCTCATATTCGTCCATCGTTTGCCTTAATTCTCGAACAGTTGACTGCTATT | |
| | GAAGGGGCAGTGATGACTGAGATGCCTCAAGAATCTTTTCATTCCATGCAAGATGACT | |
| | GGAAACTAGAAATTCAACAAATGTTTGATGAGTTGAGAACAAAGGAAAAGGAGCTGCG | |
| | ATCCCGGGAAGAGGAGCTGACTCGGGCGGCTCTGCAGCAGAAGTCTCAGGAGGAGCTG | |
| | CTAAAGCGGCGTGAGCAGCAGCTGGCAGAGCGCGAGATCGACGTGCTGGAGCGGGAAC | |
| | TTAACATTCTGATATTCCAGCTAAACCAGGAGAAGCCCAAGGTAAAGAAGAGGAAGGG | |
| | CAAGTTTAAGAGAAGTCGTTTAAAGCTCAAAGATGGACATCGAATCAGTTTACCAACA | |
| | GATTTCCAGCACAAGATAACCGTGCAGGCCTCTCCCAACTTGGACAAACGGCGGAGCC | |
| | TGAACAGCAGCAGTTCCAGTCCCCCGAGCAGCCCCACAATGATGCCCCGACTCCGAGC | |
| | CATACAGTGTGAGCTTGATGAAAGCAATAAAACTTGGGGAAGGAACACAGTCTTTCGA | |
| | CAAGAAGAATTTGAGGATGTAAAAAGGAATTTTAAGAAAAAAGGTTGTACCTGGGGAC | |
| | CAAGAAGAATTTGAGGATGTAAAAAGGAATTTTAAGAAAAAAGGTTGTACCTGGGGAC | |
| | CAAATTCCATTCAAATGAAAGATCCTAGTCAGGCCTACATTGATCTACCTCTTGGGAA | |
| | AGATGCTCAGAGAGAGAATCCTGCAGAAGCTGAAAGCTGGGAGGAGGCAGCCTCTGCG | |
| | AATGCTGCCACAGTCTCCATTGAGATGACTCCTACGAATAGTCTGAGTAGATCCCCCC | |
| | AGAGAAAGAAAACGGAGTCAGCTCTGTATGGGTGCACCGTCCTTCTGGCATCGGTGGC | |
| | TCTGGGACTGGACCTCAGAGAGCTTCATAAGCACAGGCTGCTGAAGAACCGTTGCCC | |
| | AAGGAAGAGAAGAAGAAACGAGAGGGAATCTTCCAGCGGGCTTCCAAGTCCCGCAGAA | |
| | GCGCCAGTCCTCCCACAAGCCTGCCATCCACCTGTGGGGAGGCCAGCAGCCCACCCTC | |
| | CCTGCCACTGTCAAGTGCCCTGGGCATCCTCTCCACACCTTCTTTCTCCACAAAGTGC | |
| | CTGCTGCAGATGGACAGTGAAGATCCACTGGTGGACAGTGCACCTGTCACTTGTGACT | |
| | CTGAGATGCTCACTCCGGATTTTTGTCCCACTGCCCCAGGAAGTGGTCGTGAGCCAGC | |
| | CCTCATGCCAAGACTTGACACTGATTGTAGTGTATCAAGAAACTTGCCGTCTTCCTTC | |
| | CTACAGCAGACATGTGGGAATGTACCTTACTGTGCTTCTTCAAAACATAGACCGTCAC | |
| | ATCACAGACGGACCATGTCTGATGGAAATCCGACCCCAAGTAGGTTGCTGCCACTCTG | |
| | CCCCTCACCTGCTCCTCACAGTCATCTGCCAAGGGAGGTCTCACCCAAGAAGCACAGC | |
| | ACTGTCCACATCGTGCCTCAGCGTCGCCCTGCCTCCCTGAGAAGCCGCTCAGATCTGC | |
| | CTCAGGCTTACCCACAGACAGCAGTGTCTCAGCTGGCACAGACTGCCTGTGTAGTGGG | |
| | TCGCCCAGGACCACATCCCACCCATTCCTCGCTGCCAAGGAGAGAACTAAATCCCAT | |
| | GTGCCTTCATTACTGGATGCTGACGTGGAAGGTCAGAGCAGGGACTACACTGTGCCAC | |
| | TGTGCAGAATGAGGAGCAAAACCAGCCGGCCATCTATATATGAACTGGAGAAGAATT | |
| | CCTGTCTTAAAAGTGCCTTACTGTTGTTTAAGCATTTTTTTAAGGTGAACAAATG | |
| | AACACAATGTATCTACCTTTGAACTGTTTCATGCTGCTGTGTTTTCAAAAGCTGTGGC | |
| | CATGTTCCTAAATTAGTAAGATATATCCAGCTTCTCAAAAA | |
| | ORF Start: ATG at 16 | ORF Stop: TAA at 2908 |
| | SEQ ID NO:32 | 964 aa MW at 106256.4 kD |

| | |
|---|---|
| NOV13a, | MALRGAAGATDTPVSSAGGAPGGSASSSSTSSGGSASAGAGLWAALYDYEARGEDELS |
| CG123100-01 | LRRGQLVEVLSQDAAVSGDEGWWAGQVQRRLGIFPANYVAPCRRAASPAPPPSRRSSR |
| Protein | VHVAFERLELKFLIGAGGFGQVYRATWQGQEVAVKAARQDPFQDAAAAAESVRREARL |
| Sequence | FAMLRHPNIIFLRGVCLQQPHLCLVLEFARGGALNRALAAANAAAPDPRARGPRRARRI |
| | PPHVLVNWAVQIARGMLYLHFEAFVPILHRDLLKSSNILLLEKIEHDDICNKTLKITDF |
| | GLAREWHRTTKMSTAGTYAWMAPEVIKSSLFSKGSDIWSYGVLLWEAALLTGEVPYRGID |
| | GLAVAYGVAVNKLTLRIPSTCPEPFAKLMKECWQQDPHIRPSFALILEQLTAIEGAVM |
| | TEMPQESFHSMQDDWKLEIQQMFDELRTKEKELRSREEELTRAALQQKSQEELLKRRE |
| | QQLAERAAIDVLERELNTLTFQLNQEKPKVKKRKGKFKRSRLKLKDGHRISLPTDFQHK |
| | ITVQASPNLDKRRSLNSSSSSPPSSPTMMPRLRAIQCELDESNKTWGRNTVFRQEEFE |
| | DVKRNFKKKGCTWGRNSIQMKDRSQAYIDLPLGKDAQRENPAEAESWEEAASANAATV |
| | SIEMTPTNSLSRSPQRKKTESALYGCTVLLASVALGLDLRELHKAQAAEEPLPKEEKK |
| | KREGIFQRASKSRRSASPPTSLPSTCGEASSRPSLPLSSALGILSTPSFSTKCLLQMD |
| | SEDPLVDSAPVTCDSEMLTPDFCPTAPGSGREPALMPRLDTDCSVSRNLPSSFLQQTC |
| | GNVPYCASSKHRPSHHRRTMSDGNPTPSRLLPLCPSPAPHSHLPREVSPKKHSTVHIV |
| | PQRRPASLRSRSDLPQAYPQTAVSQLAQTACVVGRPGPHPTQFLAAKERTKSHVPSLL |
| | DADVEGQSRDYTVPLCRMRSKTSRPSIYELEKEFLS |

Further analysis of the NOV13a protein yielded the following properties shown in Table 13B.

TABLE 13B
Protein Sequence Properties NOV13a

| | |
|---|---|
| PSort | 0.8500 probability located in endoplasmic reticulum |
| analysis: | (membrane); 0.8000 probability located in nucleus; 0.4400 |
| | probability located in plasma membrane; 0.3000 probability |
| | located in microbody (peroxisome) |
| SignalP | No Known Signal Sequence Predicted |
| analysis: | |

A search of the NOV13a protein against the Geneseq database, a proprietary database that contains sequences published in patents and patent publication, yielded several homologous proteins shown in Table 13C.

TABLE 13C
Geneseq Results for NOV13a

| | | | Identities/ | |
|---|---|---|---|---|
| | | NOV13a | Similarities | |
| | Protein/ | Residues/ | for the | |
| Geneseq | Organism/Length | Match | Matched | Expect |
| Identifier | [Patent #, Date] | Residues | Region | Value |
| AAB85513 | Human protein | 1 . . . 657 | 632/719 | 0.0 |
| | kinase SGK067— | | (87%) | |
| | Homo sapiens, | 1 . . . 719 | 641/719 | |
| | 719 aa. | | (88%) | |
| | [WO200155356- | | | |
| | A2, 2 AUG. | | | |
| | 2001] | | | |
| AAE21717 | Human PKIN-12 | 19 . . . 964 | 531/1115 | 0.0 |
| | protein—Homo | | (47%) | |
| | sapiens, 1097 aa. | 24 . . . 1097 | 662/1115 | |
| | [WO200218557- | | (58%) | |
| | A2, 7 MAR. | | | |
| | 2002] | | | |
| AAE11775 | Human kinase | 19 . . . 964 | 525/1069 | 0.0 |
| | (PKIN)-9 | | (49%) | |
| | protein—Homo | 24 . . . 1046 | 663/1069 | |
| | sapiens, 1046 aa. | | (61%) | |
| | [WO200181555- | | | |
| | A2, 1 NOV. | | | |
| | 2001] | | | |
| ABG11701 | Novel human | 516 . . . 951 | 389/510 | 0.0 |
| | diagnostic protein | | (76%) | |
| | #11692—Homo | 25 . . . 533 | 404/510 | |
| | sapiens, 639 aa. | | (78%) | |
| | [WO200175067- | | | |
| | A2, 11 OCT. | | | |
| | 2001] | | | |
| ABG11701 | Novel human | 516 . . . 951 | 389/510 | 0.0 |
| | diagnostic protein | | (76%) | |
| | #11692—Homo | 25 . . . 533 | 404/510 | |
| | sapiens, 639 aa | | (78%) | |
| | [WO200175067- | | | |
| | A2, 11 OCT. | | | |
| | 2001] | | | |

fit a BLAST search of public sequence databases, the NOV13a protein was found to have homology to the proteins shown in the BLASTP data in Table 13D.

TABLE 13D
Public BLASTP Results for NOV13a

| | | NOV13a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q8WWN1 | Mixed lineage | 1 . . . 964 | 928/1036 (89%) | 0.0 |
| | kinase 4beta— | 1 . . . 1036 | 941/1036 (90%) | |
| | Homo sapiens | | | |
| | (Human), | | | |
| | 1036 aa. | | | |
| Q8VDG6 | Similar to | 1 . . . 964 | 656/1035 (63%) | 0.0 |
| | mitogen-activated | 1 . . . 1001 | 735/1035 (70%) | |
| | protein kinase | | | |
| | kinase kinase 9— | | | |
| | Mus musculus | | | |
| | (Mouse), 1001 aa. | | | |
| Q9H1Y7 | DJ862P8.3 | 1 . . . 561 | 560/561 (99%) | 0.0 |
| | (Similar to | 1 . . . 561 | 561/561 (99%) | |
| | MAP3K10 | | | |
| | (Mitogen- | | | |
| | activated protein | | | |
| | kinase kinase | | | |
| | kinase 10))— | | | |
| | Homo sapiens | | | |
| | (Human), 564 aa | | | |
| | (fragment). | | | |
| Q8WWN2 | Mixed lineage | 1 . . . 561 | 558/561 (99%) | 0.0 |
| | kinase 4alpha— | 1 . . . 561 | 560/561 (99%) | |
| | Homo sapiens | | | |
| | (Human), 570 aa. | | | |
| Q9H2N5 | Mixed lineage | 43 . . . 730 | 413/701 (58%) | 0.0 |
| | kinase MLK1— | 5 . . . 675 | 511/701 (71%) | |
| | Homo sapiens | | | |
| | (Human), 1066 aa | | | |
| | (fragment). | | | |

PFam analysis predicts th at the NOV13a protein contains the domains shown in the Table 13E.

TABLE 13E
Domain Analysis of NOV13a

| Pfam | NOV13a | Identities/Similarities | Expect |
|---|---|---|---|
| Domain | Match Region | for the Matched Region | Value |
| SH3 | 41 . . . 100 | 23/63 (37%) | 3.1e−13 |
| | | 48/63 (76%) | |
| Pkinase | 124 . . . 398 | 101/314 (32%) | 4e−87 |
| | | 221/314 (70%) | |

### Example 14

The NOV14 clone as analyzed, and the nucleotide and encoded polypeptide sequences are shown in Table 14A.

TABLE 14A
NOV14 Sequence Analysis

| | SEQ ID NO:33 | 9930 bp |
|---|---|---|
| NOV14a, | CCGCGGGTGCCCCCGTGGCCGCCCAGTTCCGGCGTCCCCCCAGCCCAGCTCTCAGTGG | |
| CG124136-01 | CCATGCAGAAAGCCCGGGGCACGCGAGGCGAGGATGCGGGCACGAGGGCACCCCCCAG | |
| DNA | CCCCGGAGTGCCCCCGAAAAGGGCCAAGGTGGGGGCCGGCGGCGGGGCTCCTGTGGCC | |
| Sequence | GTGGCCGGGGCGCCAGTCTTCCTGCGGCCCCTGAAGAACGCGGCGGTGTGCGCGGGCA | |
| | GCGACGTGCGGCTGCGGGTGGTGGTGAGCGGGACGCCCCATCCCATCCTCCGCTGGTT | |
| | CCGGGATGGGCAGCTCCTGCCCGCGCCGGCCCCCGAGCCCAGCTGCCTGTGGCTGCGG | |
| | CGCTGCGGGGCGCAGGACGCCGGCGTGTACAGCTGCATGGCCCAGAACGAGCGGGGCC | |
| | GGGCCTCCTGCGAGGCGGTGCTCACAGTGCTGGAGGTCGGAGACTCAGAGACGGCTGA | |
| | GGATGACATCAGCGATGTGCAGGGAACCCAGCGCCTGGAGCTTCGGGATGACGGGGCC | |
| | TTCAGCACCCCCACGGGGGGTTCTGACACCCTGGTGGGCACCTCCCTGGACACACCCC | |
| | CGACCTCCGTGACAGGCACCTCAGAGGAGCAAGTGAGCTGGTGGGGCAGCGGGCAGAC | |
| | GGTCCTGGAGCAGGAAGCGGGCAGTGGGGGTGGCACCCGCCGCCTCCCGGGCAGCCCA | |
| | AGGCAAGCACAGGCAACCGGGGCCGGGCCACGGCACCTGGGGGTGGAGCCGCTGGTGC | |
| | GGGCATCTCGAGCTAATCTGGTGGGCGCAAGCTGGGGGTCAGAGGATAGCCTTTCCGT | |
| | GGCCAGTGACCTGTACGGCAGCGCATTCAGCCTGTACAGAGGACGGGCGCTCTCTATC | |
| | CACGTCAGCCTCCCTCAGAGCGGGTTGCGCAGGGAGGAGCCCGACCTTCAGCCTCAAC | |
| | TGGCCAGCGAAGCCCCACCCCGCCCTGCCCAGCCGCCTCCTTCCAAATCCGCGCTGCT | |
| | CCCCCCACCGTCCCCTCGGGTCGGGAAGCGGTCCCCGCCGGGACCCCCGGCCCAGCCC | |
| | GCGGCCACCCCCACGTCGCCCCACCGTCGCACTCAGGAGCCTGTGCTGCCCGAGGACA | |
| | CCACCACCGAAGAGAAGCGAGGGAAGAAGTCCAAGTCGTCCGGGCCCTCCCTGGCGGG | |
| | CACCGCGGAATCCCGACCCCAGACGCCACTGAGCGAGGCCTCAGGCCGCCTGTCGGCG | |
| | TCTTCGAGGAGCGACGGCGCAGCCTGGAGCGCAGCGACTCGCCGCCGGCGCCCCTGCG | |
| | GCCCTGGGTGCCCCTGCGCAAGGCCCGCTCTCTGGAGCAGCCCAAGTCGGAGCGCGGC | |
| | GCACCGTGGGGCACCCCCGGGGCCTCGCAGGAAGAACTGCGGGCGCCAGGCAGCGTGG | |
| | CCGAGCGGCGCCGCCTGTTCCAGCAGIAAAGCGGCCTCGCTGGACGAGCGCACGCGTCA | |
| | GCGCAGCCCGGCCTCAGACCTCGAGCTGCGCTTCGCCCAGGAGCTGGGCCGCATCCGC | |
| | CGCTCCACGTCGCGGGAGGAGCTGGTGCGCTCGCACGAGTCCCTGCGCGCCACGCTGC | |
| | AGCGTGCCCCATCCCCTCGAGAGCCCGGCGAGCCCCCGCTCTTCTCTCGGCCCTCCAC | |
| | CCCCAAGACATCGCGGGCCGTGAGCCCCGCCGCCGCCCAGCCGCCCTCTCCGAGCAGC | |
| | GCGGAGAAGCCGGGGGACGAGCCTGGGAGGCCCAGGAGCCGCGGGCCGGCGGGCAGGA | |
| | CAGAGCCGGGGGAAGGCCCGCAGCAGGAGGTTAGGCGTCGGGACCAATTCCCGCTGAC | |
| | CCGGAGCAGAGCCATCCAGGAGTGCAGGAGCCCTGTGCCGCCCCCCGCCGCCGATCCC | |
| | CCAGAGGCCAGGACGAAAGCACCCCCCGGTCGGAAGCCGGAGCCCCCGGCGCAGGCCG | |
| | TGCGCTTCCTGCCCTGGGCCACGCCGGGCCTGGAGGGCGCTGCTGTACCCCAGACCTT | |
| | GGAGAAGAACAGGGCGGGGCCTGAGGCAGAGAAGAGGCTTCGCAGAGGGCCGGAGGAG | |
| | GACGGTCCCTGGGGGCCCTGGGACCGCCGAGGGGCCCGCAGCCAGGGCAAAGGTCGCC | |
| | GGGCCCGGCCCACCTCCCCTGAGCTCGAGTCTTCGGATGACTCCTACGTGTCCGCTGG | |
| | AGAAGAGCCCCTAGAGGCCCCTGTGTTTGAGATCCCCCTGCAGAATGTGGTGGTGGCA | |
| | CCAGGGGCAGATGTGCTGCTCAAGTGTATCATCACTGCCAACCCCCCGCCCCAAGTGT | |
| | CCTGGCACAAGGATGCGTCAGCGCTGCGCAGCGAGGGCCGCCTCCTCCTCCGGGCTGA | |
| | GGGTGAGCGGCACACCCTGCTGCTCAGGGAGGCCAGGGCAGCAGATGCCGGGAGCTAT | |
| | ATGGCCACCGCCACCAACGAGCTGGGCCAGGCCACCTGTGCCGCCTCACTGACCCTGA | |
| | GACCCGGTGGGTCTACATCCCCTTTCAGCAGCCCCATCACCTCCGACGAGGAATACCT | |
| | GAGCCCCCCAGAGGAGTTCCCAGAGCCTGGGGAGACCTGGCCGCGAACCCCCACCATG | |
| | AAGCCCAGTCCCAGCCAGAACCGCCGTTCTTCTGACACTGGCTCCAAGGCACCCCCCA | |
| | CCTTCAAGGTCTCACTTATGGACCAGTCAGTAAGAGAAGGCCAAGATGTCATCATGAG | |
| | CATCCGCGTGCAGGGGGAGCCCAAGCCTGTGGTCTCCTGGCTGAGAAACCGCCAGCCC | |
| | GTGCGCCCAGACCAGCGGCGCTTTGCGGAGGAGGCTGAGGGTCGGCTCTGCCGGCTGC | |
| | GGATCCTGGCTGCAGAGCGTGGCGATGCTGGTTTCTACACTTGCAAAGCGGTCAATGA | |
| | GTATGGTGCTCGGCAGTGCGAGGCCCGCTTGAGGTCCGAGGACGTGGACGTGGGGGCC | |
| | GGGGAGATGGCGCTGTTTGAGTGCCTGGTGGCGGGGCCCACTGACGTGGAGGTGGATT | |
| | GGCTGTGCCGTGGCCGCCTGCTGCAGCCTGCACTGCTCAAATGCAAGATGCATTTCGA | |
| | TGGCCGCAAATGCAAGCTGCTACTTACATCTGTACATGAGGACGACAGTGGCGTCTAC | |
| | ACCTGCAAGCTCAGCACGGCCAAAGATGAGCTGACCTGCAGTGCCCGCCTGACCGTGC | |
| | GGCCCTCGTTGGCACCCCTGTTCACACGGCTGCTGGAAGATGTGGAGGTGTTGGAGGG | |
| | CCGAGCTGCCCCTTTCGACTGCAAGATCAGTGGCACCCCGCCCCCTGTTGTTACCTGG | |
| | ACTCATTTTGGCTGCCCCATGGAGGAGAGTGAGAACTTGCGGCTGCGGCAGGACGGGG | |
| | GTCTGCACTCACTGCACATTGCCCATGTGGCCAGCGAGGACGAGGGGCTCTATGCGGT | |
| | CAGTGCTGTTAACACCCATGGCCAGGCCCACTGCTCAGCCCAGCTCTATGTAGAAGAG | |
| | CCCCGGACAGCCGCCTCAGGCCCCAGCTCCAAGCTGGAGAAGATGCCATCCATTCCCG | |
| | AGGAGCCAGAGCAGGGTGAGCTGGAGCGGCTGTCCATTCCTGACTTCCTGCGGCCACT | |
| | GCAGGACCTGGAGGTGGGACTGGCCAAGGAGGCCATGCTAGAGTGCCAGGTGACCGGC | |
| | CTGCCCTACCCCACCATCAGCTGGTTCCACAATGGCCACCGCATCCAGAGCAGCGACG | |
| | ACCGGCGCATGACACAGTACAGGGATGTCCATCGCTTGGTGTTCCCTGCCGTGGGGCC | |
| | TCAGCACGCCGGTGTCTACAAGAGCGTCATTGCCAACAAGCTGGGCAAAGCTGCCTGC | |
| | TATGCCCACCTGTATGTCACAGATGTGGTCCCAGGCCCTCCAGATGGCGCCCCGCAGG | |
| | TGGTGGCTGTGACGGGGAGGATGGTCACACTCACATGGAACCCCCCCAGGAGTCTGGA | |
| | CATGGCCATCGACCCGGACTCCCTGACGTACACAGTGCAGCACCAGGTGCTGGGCTCG | |
| | GACCAGTGGACGGCACTGGTCACAGGCCTGCGGGAGCCAGGGTGGGCAGCCACAGGGC | |
| | TGCGTAAGGGGGTCCAGCACATCTTCCGGGTCCTCAGCACCACTGTCAAGAGCAGCAG | |
| | CAAGCCCTCACCCCCTTCTGAGCCTGTGCAGCTGCTGGAGCACGGCCCAACCCTGGAG | |
| | GAGGCCCCTGCCATGCTGGACAAACCAGACATCGTGTATGTGGTGGAGGGACAGCCTG | |
| | CCAGCGTCACCGTCACATTCAACCATGTGGAGGCCCAGGTCGTCTGGAGGAGCTGCCG | |
| | AGGGGCCCTCCTAGAGGCACGGGCCGGTGTGTACGAGCTGAGCCAGCCAGATGATGAC | |
| | CAGTACTGTCTTCGGATCTGCCGGGTGAGCCGCCGGGACATGGGGGCCCTCACCTGCA | |
| | CCGCCCGAAACCGTCACGGCACACAGACCTGCTCGGTCACATTGGAGCTGGCAGAGGC | |
| | CCCTCGGTTTGAGTCCATCATGGAGGACGTGGAGGTGGGGGCTGGGGAAACTGCTCGC | |
| | TTTGCGGTGGTGGTCGAGGGAAAACCACTGCCGGACATCATGTGGTACAAGGACGAGG | |
| | TGCTGCTGACCGAGAGCAGCCATGTGAGCTTCGTGTACGAGGAGAATGAGTGCTCCCT | |
| | GGTGGTGCTCAGCACGGGGGCCCAGGATGGAGGCGTCTACACCTGCACCGCCCAGAAC | |
| | CTGGCGGGTGAGGTCTCCTGCAAAGCAGAGTTGGCTGTGCATTCAGCTCAGACAGCTA | |
| | TGGAGGTCGAGGGGGTCGGGGAGGATGAGGACCATCGAGGAAGGAGACTCAGCGACTT | |
| | TTATGACATCCACCAGGAGATCGGCAGGGGTGCTTTCTCCTACTTGCGGCGCATAGTG | |
| | GAGCGTAGCTCCGGCCTGGAGTTTGCGGCCAAGTTCATCCCCAGCCAGGCCAAGCCAA | |
| | AGGCATCAGCGCGTCGGGAGGCCCGGCTGCTGGCCAGGCTCCAGCACGACTGTGTCCT | |
| | CTACTTCCATGAGGCCTTCGAGAGGCGCCGGGGACTGGTCATTGTCACCGAGCTCTGC | |
| | ACAGAGGAGCTGCTGGAGCGAATCGCCAGGAAACCCACCGTGTGTGAGTCTGAGATCC | |
| | GGGCCTATATGCGGCAGGTGCTAGAGGGAATACACTACCTGCACCAGAGCCACGTGCT | |
| | GCACCTCGATGTCAAGCCTGAGAACCTGCTGGTGTGGGATGGTGCTGCGGGCGAGCAG | |
| | CAGGTGCGGATCTGTGACTTTGGGAATGCCCAGGAGCTGACTCCAGGAGAGCCCCAGT | |
| | ACTGCCAGTATGGCACACCTGAGTTTGTAGCACCCGAGATTGTCAATCAGAGCCCCGT | |
| | GTCTGGAGTCACTGACATCTGGCCTGTGGGTGTTGTTGCCTTCCTGCTGTCTGACAGG | |
| | AATCTCCCCGTTTGTTGGGGAAATGACCGGACAACATTGATGAACATCCGAAACTACA | |
| | ACGTGGCCTTCGAGGAGACCACATTCCTGAGCCTGAGCAGGGAGGCCCGGGGCTTCCT | |
| | CATCAAAGTGTTGGTGCAGGACCGGCTGAGACCTACCGCAGAAGAGACCCTAGAACAT | |
| | CCTTGGTTCAAAACTCAGGCAAAGGGCGCAGAGGTGAGCACGGATCACCTGAAGCTAT | |
| | TCCTCTCCCGGCGGAGGTGGCAGCGCTCCCAGATCAGCTACAAATGCCACCTGGTGCT | |
| | GCGCCCCATCCCCGAGCTGCTGCGGGCCCCCCCAGAGCGGGTGTGGGTGACCATGCCC | |
| | AGAAGGCCACCCCCCAGTGGGGGGCTCTCATCCTCCTCGGATTCTGAAGAGGAAGAGC | |
| | TGGAAGAGCTGCCCTCAGTGCCCCGCCCACTGCAGCCCGAGTTCTCTGGCTCCCGGGT | |
| | GTCCCTCACAGACATTCCCACTGAGGATGAGGCCCTGGGGACCCCAGAGACTGGGGCT | |
| | GCCACCCCCATGGACTGGCAGGAGCAGGGAAGGGCTCCCTCTCAGGACCAGGAGGCTC | |
| | CCAGCCCAGAGGCCCTCCCCTCCCCAGGCCAGGAGCCCGCAGCTGGGGCTAGCCCCAG | |
| | GCGGGGAGAGCTCCGCAGGGGCAGCTCGGCTGAGAGCGCCCTGCCCCGGGCCGGGCCG | |
| | CGGGAGCTGGGCCGGGGCCTGCACAAGGCGGCGTCTGTGGAGCTGCCGCAGCGCCGGA | |
| | GCCCCGGCCCGGGAGCCACCCGCCTGGCCCGGGGAGGCCTGGGTGAGGGCGAGTATGC | |
| | CCAGAGGCTGCAGGCCCTGCGCCAGCGGCTGCTGCGGGGAGGCCCCGAGGATGGCAAG | |
| | GTCAGCGGCCTCAGGGGTCCCCTGCTGGAGAGCCTGGGGGGCCGTGCTCGGGACCCCC | |
| | GGATGGCACGAGCTGCCTCCAGCGAGGCAGCGCCCCACCACCAGCCCCCACTCGAGAA | |
| | CCGGGGCCTGCAAAAGAGCAGCAGCTTCTCCCAGGGTGAGGCGGAGCCCCGGGGCCGG | |
| | CACCGCCGAGCGGGGGCGCCCCTCGAGATCCCCGTGGCCAGGCTTGGGGCCCGTAGGC | |
| | TACAGGAGTCTCCTTCCCTGTCTGCCCTCAGCGAGGCCCAGCCATCCAGCCCTGCACG | |
| | GCCCAGCGCCCCCAAACCCAGTACCCCTAAGTCTGCAGAACCTTCTGCCACCACACCT | |
| | AGTGATGCTCCGCAGCCCCCCGCACCCCAGCCTGCCCAAGACAAGGCTCCAGAGCCCA | |
| | GGCCAGAACCAGTCCGAGCCTCCAAGCCTGCACCACCCCCCCAGGCCCTGCAAACCCT | |
| | AGCGCTGCCCCTCACACCCTATGCTCAGATCATTCAGTCCCTCCAGCTGTCAGGCCAC | |
| | GCCCAGGGCCCCTCGCAGGGCCCTGCCGCGCCGCCTTCAGAGCCCAAGCCCCACGCTG | |
| | CTGTCTTTGCCAGGGTGGCCTCCCCACCTCCGGGAGCCCCCGAGAAGCGCGTGCCCTC | |
| | AGCCGGGGGTCCCCCGGTGCTAGCCGAGAAAGCCCGAGTTCCCACGGTGCCCCCCAGG | |
| | CCAGGCAGCAGTCTCAGTAGCAGCATCGAAAACTTGGAGTCGGAGGCCGTGTTCGAGG | |
| | CCAAGTTCAAGCGCAGCCGCGAGTCGCCCCTGTCGCTGGGGCTGCGGCTGCTGAGCCG | |
| | TTCGCGCTCGGAGGAGCGCGGCCCCTTCCGTGGGGCCGAGGAGGAGGATGGCATATAC | |
| | CGGCCCAGCCCGGCGGGGACCCCGCTGGAGCTGGTGCGACGGCCTGAGCGCTCACGCT | |
| | CGGTGCAGGACCTCAGGGCTGTCGGAGAGCCTGGCCTCGTCCGCCGCCTCTCGCTGTC | |
| | ACTGTCCCAGCGGCTGCGGCGGACCCCTCCCGCGCAGCGCCACCCGGCCTGGGAGGCC | |
| | CGCGGCGGGGACGGAGAGAGCTCGGAGGGCGGGAGCTCGGCGCGGGGCTCCCCGGTGC | |
| | TGGCGATGCGCAGGCGGCTGAGCTTCACCCTGGAGCGGCTGTCCAGCCGATTGCAGCG | |
| | CAGTGGCAGCAGCGAGGACTCGGGGGGCGCGTCGGGCCGCAGCACGCCGCTGTTCGGA | |
| | CCGCTTCGCAGGGCCACGTCCGAGGGCGAGAGTCTGCGGCGCCTTGGCCTTCCGCACA | |
| | ACCAGTTGGCCGCCCAGGCCGGCGCCACCACGCCTTCCGCCGAGTCCCTGGGCTCCGA | |
| | GGCCAGCGCCACGTCGGGCTCCTCAGCCCCAGGGGAAAGCCGAAGCCGGCTCCGCTGG | |
| | GGCTTCTCTCGGCCGCGGAAGGACAAGGGGTTATCGCCACCAPACCTCTCTGCCAGCG | |
| | TCCAGGAGGAGTTGGGTCACCAGTACGTGCGCAGTGAGTCAGACTTCCCCCCAGTCTT | |
| | CCACATCAAACTCAAGGACCAGGTGCTGCTGGAGGGGGAGGCAGCCACCCTGCTCTGC | |
| | CTGCCAGCGGCCTGCCCTGCACCGCACATCTCCTGGATGAAAGACAAGAAGTCCTTGA | |
| | GGTCAGAGCCCTCAGTGATCATCGTGTCCTGCAAAGATGGGCGGCAGCTGCTCAGCAT | |
| | CCCCCGGGCGGGCAAGCGGCACGCCGGTCTCTATGAGTGCTCGGCCACCAACGTACTG | |
| | GGCAGCATCACCAGCTCCTGTACCGTGGCTGTGGCCCGAGTCCCAGGAAAGCTAGCTC | |
| | CTCCAGAGGTACCCCAGACCTACCAGGACACGGCGCTGGTGCTGTGGAAGCCGGGAGA | |
| | CAGCCGGGCACCTTGCACGTATACGCTGGAGCGGCGAGTGGATGGGGAGTCTGTGTGG | |
| | CACCCTGTGAGCTCAGGCATCCCCGACTGTTACTACAACGTGACCCACCTGCCAGTTG | |
| | GCGTGACTGTGAGGTTCCGTGTGGCCTGTGCCAACCGTGCTGGGCAGGGGCCCTTCAG | |
| | CAACTCTTCTGAGAAGGTCTTTGTCAGGGGTACTCAAGATTCTTCAGCTGTGCCATCT | |
| | GCTGCCCACCAAGAGGCCCCTGTCACCTCAAGGTCAGTCAGGGCCCGGCCTCCTGACT | |
| | CTCCTACCTCACTGGCCTCACCCCTAGCTCCTGCTGCCCCCACACCCCCGTCAGTCAC | |
| | TGTCAGCCCCTCATCTCCCCCCACACCTCCTAGCCAGGCCTTGTCCTCGCTCAAGGCT | |
| | GTGGGTCCACCACCCCAAACCCCTCCACGAAGACACAGGGGCCTGCAGGCTGCCCGGC | |
| | CAGCGGAGCCCACCCTACCCAGTACCCACGTCACCCCAAGTGAGCCCCAGCCTTTCGT | |
| | CCTTGACACTGGGACCCCGATCCCAGCCTCCACTCCTCAAGGGGTTAAACCAGTGTCT | |
| | TCCTCTACTCCTGTGTATGTGGTGACTTCCTTTGTGTCTGCACCACCAGCCCCTGAGC | |
| | CCCCAGCCCCTGAGCCCCCTCCTGAGCCTACCAAGGTGACTGTGCAGAGCCTCAGCCC | |
| | GGCCAAGGAGGTGGTCAGCTCCCCTGGGAGCAGTCCCCGkAGCTCTCCCAGGCCTGAG | |
| | GGTACCACTCTTCGACAGGGTCCCCCTCAGkAACCCTACACCTTCCTGGAGGAGAAAG | |
| | CCAGGGGCCGCTTTGGTGTTGTGCGAGCGTGCCGGGAGAATGCCACGGGGCGAACGTT | |
| | CGTGGCCAAGATCGTGCCCTATGCTGCCGAGGGCAAGCCGCGGGTCCTGCAGGAGTAC | |
| | GAGGTGCTGCGGACCCTGCACCACGAGCGGATCGTGTCCCTGCACGAGGCCTACATCA | |
| | CCCCTCGGTACCTCGTGCTCATTGCTGAGAGCTGTGGCAACCGGGAACTCCTCTGTGG | |
| | GCTCAGTGACAGGTTCCGGTATTCTGAGGATGACGTGGCCACTTACATGGTGCAGCTG | |
| | CTACAAGGCCTGGACTACCTCCACGGCCACCACGTCCTCCACCTAGACATCAAGCCAG | |
| | ACAACCTGCTGCTGGCCCCTGACAATGCCCTCAAGATTGTGGACTTTGGCAGTGCCCA | |
| | GCCCTACAACCCCCAGGCCCTTAGGCCCCTTGGCCACCGCACGGTGCACCTGACACTA | |
| | ATGTCCTTCTGGGTCTGGGTGTTGGCCTCCGGTCTGCATATGTCAATCAAGCTATCTT | |
| | CCCCAACAGGCTCAGTGGACGCTCCCCGTTCTATGAGCCAGACCCCCAGGAAACGGAG | |
| | GCTCGGATTGTGGGGGGCCGCTTTGATGCCTTCCAGCTGTACCCCAATACATCCCAGA | |
| | GCGCCACCCTCTTCTTGCGAAAGGTTCTCTCTGTACATCCCTGGTGAGTGAGCCCCAC | |
| | ACCTGCTATCCCCCAGTGTTACCTGCCCCTGGCCTGGCCTGTGCCAGAGATCTCCCAG | |
| | CTCCTCCCCTGCTCCTAGGAAGAAGTCTGCTGCTTCTACTAAATGGTCATACTACCCA | |
| | CCATTTAAAGCCTGAGGCAGCCCCGTGCAAGGCAGACTCACTGTCCCCATTCCGGCGA | |
| | CTGGGGAACTGAGCTCTTGAGCTGCCCAAGATCACACATGTAGGGGTGGGATCCAGGA | |
| | CTGGGACATGGGTCTGCGGGAGGACAGAGCCCCGGCAGCTCCCAGAGCTTCCTTCCAG | |
| | GTTCATCATCCC | |
| | ORF Start: ATG at 61 | ORF Stop: TGA at 9619 |
| | SEQ ID NO:34 | 3186 aa MW at 344940.7 kD |
| NOV14a, | MQKARGTRGEDAGTRAPPSPGVPPKRAKVGAGGGAPVAVAGAPVFLRPLKNAAVCAGS | |
| CG124136-01 | DVRLRVVVSGTPHPILRWFRDGQLLPARAPEPSCLWLRRCGAQDAGVYSCMAQNERGR | |
| Protein | ASCEAVLTVLEVGDSETAEDDISDVQGTQRLELRDDGAFSTPTGGSDTLVGTSLDTPP | |
| Sequence | TSVTGTSEEQVSWWGSGQTVLEQEAGSGGGTPRLPGSPRQAQATGAGPRHLGVEPLVR | |
| | ASRANLVGASWGSEDSLSVASDLYGSAFSLYRGRALSIHVSVPQSGLRREEPDLQPQL | |
| | ASEAPRRPAQPPPSKSALLPPPSPRVGKRSPPGPPAQPAATPTSPHRRTQEPVLPEDT | |
| | TTEEKRGKKSKSSGPSLAGTAESRPQTPLSEASGRLSALGRSPRLVRAGSRILDKLQF | |
| | FEERRRSLERSDSPPAPLRPWVPLRKARSLEQPKSERGAPWGTPGASQEELRAPGSVA | |
| | ERRRLFQQKAASLDERTRQRSPASDLELRFAQELGRIRRSTSREELVRSHESLRATLQ | |
| | RAPSPREPGEPPLFSRPSTPKTSRAVSPAAAQPPSPSSAEKPGDEPGRPRSRGPAGRT | |
| | EPGFGPQQEVRRRDQFPLTPSRAIQECRSPVPPPAADRPEARTKAPPGRKREPPAQAV | |
| | RFLPWATPGLEGAAVPQTLEKNRAGPEAEKRLRRGPEEDGPWGPWDRRGARSQGKGRR | |
| | ARPTSPELESSDDSYVSAGEEPLEAPVFEIPLQNVVVAPGADVLLKCIITANPPPQVS | |
| | WHKDGSALRSEGRLLLRAEGERHTLLLREARAADAGSYMATATNELGQATCAASLTVR | |
| | PGGSTSPFSSPITSDEEYLSPPEEFPEPGETWPRTPTMKPSRSQNRRSSDTGSAAPPT | |
| | FKVSLMDQSVREGQDVIMSIRVQGEPKPVVSWLRNRQPVRPDQRRFAEEAEGGLCRLR | |
| | ILAAERGDAGFYTCKAVNEYGARQCEARLRSEDVDVGAGEMALFECLVAGPTDVEVDW | |
| | LCRGRLLQPALLKCKMHFDGRKCKLLLTSVHEDDSGVYTCKLSTAKDELTCSARLTVR | |
| | PSLAPLFTRLLEDVEVLEGRAARFDCKISGTPPPVVTWTHFGCPMEESENLRLRQDGG | |
| | LHSLHIAHVGSEDEGLYAVSAVNTHGQAHCSAQLYVEEPRTAASGPSSKLEKMPSIPE | |
| | EPEQGELERLSIPDFLRPLQDLEVGLAKEAMLECQVTGLPYPTTSWPHNGHRIQSSDD | |
| | RRMTQYRDVHRLVFPAVGPQHAGVYKSVIANKLGKAACYAHLYVTDVVPGPPDGAPQV | |
| | VAVTGRMVTLTWNPPPSLDMAIDPDSLTYTVQHQVLGSDQWTALVTGLREPGWAATGL | |
| | RKGVQHIPRVLSTTVKSSSKPSPPSEPVQLLEHGPTLEFAPAMLDKPDIVYVVEGQPA | |
| | SVTVTFNHVFAQVVWRSCRGALLEARAGVYELSQPDDDQYCLRICRVSRRDMGALTCT | |
| | ARNRHGTQTCSVTLELAEAPRFESIMEDVEVGAGETARFAVVVEGKPLPDIMWYKDEV | |
| | LLTESSHVSFVYEENECSLVVLSTGAQDGGVYTCTAQNLAGEVSCAAELAVHSAQTAM | |
| | EVEGVGEDEDHRGRRLSDFYDIHQEIGRGAFSYLRRIVERSSGLEFAAKFIPSQAKPK | |
| | ASARREARLLARLQHDCVLYFHEAFERRRGLVIVTELCTEELLERIARKPTVCESEIR | |
| | AYMRQVLEGIHYLHQSHVLHLDVKPENLLVWDGAAGEQQVRICDFGNAQELTPGEPQY | |
| | CQYGTPEFVAPEIVNQSPVSGVTDIWPVGVVAFLLSDRNLPVCWGNDRTTLMNIRNYN | |
| | VAFEETTFLSLSREARGFLIKVLVQDRLRPTAEETLEHPWFKTQAKGAEVSTDHLKLF | |
| | LSRRRWQRSQISYKCHLVLRPIPELLRAPPERVWVTMPRRPPPSGGLSSSSDSEEEEL | |
| | EELPSVPRPLQPEFSGSRVSLTDIPTEDEALGTPETGAATPMDWQEQGRAPSQDQEAP | |
| | SPEALPSPGQEPAAGASPRRGELRRGSSAESALPRAGPRELGRGLHKAASVELPQRRS | |
| | PGPGATRLARGGLGEGEYAQRLQALRQRLLRGGPEDGKVSGLRGPLLESLGGRARDPR | |
| | MARAASSEAAPHHQPPLENRGLQKSSSFSQGEAEPRGRHRRAGAPLETPVARLGARRL | |
| | QESPSLSALSEAQPSSPARPSAPKPSTPKSAEPSATTPSDAPQPPAPQPAQDKAPEPR | |
| | PEPVRASKPAPPPQALQTLALPLTPYAQIIQSLQLSGHAQGPSQGPAAPPSEPKPHAA | |
| | VFARVASPPPGAPEKRVPSAGGPPVLAEKARVPTVPPRPGSSLSSSIENLESEAVFEA | |
| | KFKRSRESPLSLGLRLLSRSRSEERGPFRGAEEEDGIYRPSPAGTPLELVRRPERSRS | |
| | VQDLRAVGEPGLVRRLSLSLSQRLRRTPPAQRHPAWEARGGDGESSEGGSSARGSPVL | |
| | AMRRRLSFTLERLSSRLQRSGSSEDSGGASGRSTPLFGRLRRATSEGESLRRLGLPHN | |
| | QLAAQAGATTPSAESLGSEASATSGSSAPGESRSRLRWGFSRPRKDKGLSPPNLSASV | |
| | QEELGHQYVRSESDFPPVFHIKLKDQVLLEGEAATLLCLPAACPAPHISWMKDKKSLR | |
| | SEPSVITVSCKDGRQLLSIPRAGKRHAGLYECSATNVLGSITSSCTVAVARVPGKLAP | |
| | PEVPQTYQDTALVLWKPGDSRAPCTYTLERRVDGESVWHPVSSGIPDCYYNVTHLPVG | |
| | VTVRFRVACANRAGQGPFSNSSEKVFVRGTQDSSAVPSAAHQEAPVTSRSVRARPPDS | |
| | PTSLASPLAPAAPTPPSVTVSPSSPPTPPSQALSSLKAVGPPPQTPPRRHRGLQAARP | |
| | AEPTLPSTHVTPSEPQPFVLDTGTPIPASTPQGVKPVSSSTPVYVVTSFVSAPPAPEP | |
| | PAPEPPPEPTKVTVQSLSPAKEVVSSPGSSPRSSPRPEGTTLRQGPRQKPYTFLEEKA | |
| | RGRFGVVRACRENATGRTFVAKTVPYAAEGKPRVLQEYEVLRTLHHERTVSLHEAYIT | |
| | PRYLVLIAESCGNRELLCGLSDRFRYSEDDVATYMVQLLQGLDYLHGHHVLHLDIKPD | |
| | NLLLAPDNALKIVDFGSAQPYNPQALRPLGHRTVHLTLMSFWVWVLASGLHMSIKLSS | |
| | PTGSVDAPRSMSQTPRKRRLGLWGAALMPSSCTPIHPRAPPSSCERFSLYIPGE | |
| | SEQ ID NO:35 | 10122 bp |
| NOV14b, | CCGCGGGTGCCCCCGTGGCCGCCCAGTTCCGGCGTCCCCCCAGCCCAGCTCTCAGTGG | |
| CG124136-02 | CCATGCAGAAAGCCCGGGGCACGCGAGGCGAGGATGCGGGCACGAGGGCACCCCCCAG | |
| DNA | CCCCGGAGTGCCCCCGAAAAGGGCCAAGGTGGGGGCCGGCGGCGGGGCTCCTGTGGCC | |
| Sequence | GTGGCCGGGGCGCCAGTCTTCCTGCGGCCCCTGAAGAACGCGGCGGTGTGCGCGGGCA | |
| | GCGACGTGCGGCTGCGGGTGGTGGTGAGCGGGACGCCCCATCCCATCCTCCGCTGGTT | |
| | CCGGGATGGGCAGCTCCTGCCCGCGCCGGCCCCCGAGCCCAGCTGCCTGTGGCTGCGG | |
| | CGCTGCGGGGCGCAGGACGCCGGCGTGTACAGCTGCATGGCCCAGAACGAGCGGGGCC | |
| | GGGCCTCCTGCGAGGCGGTGCTCACAGTGCTGGAGGTCGGAGACTCAGAGACGGCTGA | |
| | GGATGACATCAGCGATGTGCAGGGAACCCAGCGCCTGGAGCTTCGGGATGACGGGGCC | |
| | TTCAGCACCCCCACGGGGGGTTCTGACACCCTGGTGGGCACCTCCCTGGACACACCCC | |
| | CGACCTCCGTGACAGGCACCTCAGAGGAGCAAGTGAGCTGGTGGGGCAGCGGGCAGAC | |
| | GGTCCTGGAGCAGGAAGCGGGCAGTGGGGGTGGCACCCGCCGCCTCCCGGGCAGCCCA | |
| | AGGCAAGCACAGGCAACCGGGGCCGGGCCACGGCACCTGGGGGTGGAGCCGCTGGTGC | |
| | GGGCATCTCGAGCTAATCTGGTGGGCGCAAGCTGGGGGTCAGAGGATAGCCTTTCCGT | |
| | GGCCAGTGACCTGTACGGCAGCGCATTCAGCCTGTACAGAGGACGGGCGCTCTCTATC | |
| | CACGTCAGCGTCCCTCAGAGCGGGTTGCGCAGGGAGGAGCCCGACCTTCAGCCTCAAC | |
| | TGGCCAGCGAAGCCCCACGCCGCCCTGCCCAGCCGCCTCCTTCCAAATCCGCGCTGCT | |
| | CCCCCCACCGTCCCCTCGGGTCGGGAAGCGGTCCCCGCCGGGACCCCCGGCCCAGCCC | |
| | GCGGCCACCCCCACGTCGCCCCACCGTCGCACTCAGGAGCCTGTGCTGCCCGAGGACA | |
| | CCACCACCGAAGAGAAGCGAGGGAAGAAGTCCAAGTCGTCCGGGCCCTCCCTGCCGGG | |
| | CACCGCGGAATCCCGACCCCAGACGCCACTGAGCGAGGCCTCAGGCCGCCTGTCGGCG | |
| | TTGGGCCGATCGCCTAGGCTGGTGCGCGCCGGCTCCCGCATCCTGGACAAGCTGCAGT | |
| | TCTTCGAGGAGCGACGGCGCAGCCTGGAGCGCAGCGACTCGCCGCCGGCGCCCCTGCG | |
| | GCCCTCGGTGCCCCTGCGCAAGGCCCGCTCTCTGGAGCAGCCCAAGTCGGAGCGCGGC | |
| | GCACCGTGGGGCACCCCCGGGGCCTCGCAGGAAGAACTGCGGGCGCCAGGCAGCGTGG | |
| | CCGAGCGGCGCCGCCTGTTCCAGCAGAAAGCGGCCTCGCTGGACGAGCGCACGCGTCA | |
| | GCGCAGCCCGGCCTCAGACCTCGAGCTGCGCTTCGCCCAGGAGCTGGGCCGCATCCGC | |
| | CGCTCCACGTCGCGGGAGGAGCTGGTGCGCTCGCACGAGTCCCTGCGCGCCACGCTGC | |
| | AGCGTGCCCCATCCCCTCGAGAGCCCGGCGAGCCCCCGCTCTTCTCTCGGCCCTCCAC | |
| | CCCCAAGACATCGCGGGCCGTGAGCCCCGCCGCCGCCCAGCCGCCCTCTCCGAGCAGC | |
| | GCGGAGAAGCCGGGGGACGAGCCTGGGAGGCCCAGGAGCCGCGGGCCGGCGGGCAGGA | |
| | CAGAGCCGGGGGAAGGCCCGCAGCAGGAGGTTAGGCGTCGGGACCAATTCCCGCTGAC | |
| | CCGGAGCAGAGCCATCCAGGAGTGCAGGAGCCCTGTGCCGCCCCCCGCCGCCGATCCC | |
| | CCAGAGGCCAGGACGAAAGCACCCCCCGGTCGGAAGCGGGAGCCCCCGGCGCAGGCCG | |
| | TGCGCTTCCTGCCCTGGGCCACGCCGGGCCTGGAGGGCGCTGCTGTACCCCAGACCTT | |
| | GGAGAAGAACAGGGCGGGGCCTGAGGCAGAGAAGAGGCTTCGCAGAGGGCCGGAGGAG | |
| | GACGGTCCCTGGGGGCCCTGGGACCGCCGAGGGGCCCGCAGCCAGGGCAAAGGTCGCC | |
| | GGGCCCGGCCCACCTCCCCTGAGCTCGAGTCTTCGGATGACTCCTACGTGTCCGCTGG | |
| | AGAAGAGCCCCTAGAGGCCCCTGTGTTTGAGATCCCCCTGCAGAATGTGGTGGTGGCA | |
| | CCAGGGGCAGATGTGCTGCTCAAGTGTATCATCACTGCCAACCCCCCGCCCCAAGTGT | |
| | CCTGGCACAAGGATGGGTCAGCGCTGCGCAGCGAGGGCCGCCTCCTCCTCCGGGCTGA | |
| | GGGTGAGCGGCACACCCTGCTGCTCAGGGAGGCCAGGGCAGCAGATGCCGGGAGCTAT | |
| | ATGGCCACCGCCACCAACGAGCTGGGCCAGGCCACCTGTGCCGCCTCACTGACCGTGA | |
| | GACCCGGTGGGTCTACATCCCCTTTCAGCAGCCCCATCACCTCCGACGAGGAATACCT | |
| | GAGCCCCCCAGAGGAGTTCCCAGAGCCTGGGGAGACCTGGCCGCGAACCCCCACCATG | |
| | AAGCCCAGTCCCAGCCAGAACCGCCGTTCTTCTGACACTGGCTCCAAGGCACCCCCCA | |
| | CCTTCAAGGTCTCACTTATGGACCAGTCAGTAAGAGAAGGCGAAGATGTCATCATGAG | |
| | CATCCGCGTGCAGGGGGAGCCCAAGCCTGTGGTCTCCTGGCTGAGAAACCGCCAGCCC | |
| | GTGCGCCCAGACCAGCGGCGCTTTGCGGAGGAGGCTGAGGGTGGGCTGTGCCGGCTGC | |
| | GGATCCTGGCTGCAGAGCGTGGCGATGCTGGTTTCTACACTTGCAAAGCGGTCAATGA | |
| | GTATGGTGCTCGGCAGTGCGAGGCCCGCTTGAGGTCCGAGGACGTGGACGTGGGGGCC | |
| | GGGGAGATGGCGCTGTTTGAGTGCCTGGTGGCGGGGCCCACTGACGTGGAGGTGGATT | |
| | GGCTGTGCCGTGGCCGCCTGCTGCAGCCTGCACTGCTCAAATGCAAGATGCATTTCGA | |
| | TGGCCGCAAATGCAAGCTGCTACTTACATCTGTACATGAGGACGACAGTGGCGTCTAC | |
| | ACCTGCAAGCTCAGCACGGCCAAAGATGAGCTGACCTGCAGTGCCCGGCTGACCGTGC | |
| | GGCCCTCGTTGGCACCCCTGTTCACACGGCTGCTGGAAGATGTGGAGGTGTTGGAGGG | |
| | CCGAGCTGCCCGTTTCGACTGCAAGATCAGTGGCACCCCGCCCCCTGTTGTTACCTGG | |
| | ACTCATTTTGGCTGCCCCATGGAGGAGAGTGAGAACTTGCGGCTGCGGCAGGACGGGG | |
| | GTCTGCACTCACTGCACATTGCCCATGTGGGCAGCGAGGACGAGGGGCTCTATGCGGT | |
| | CAGTGCTGTTAACACCCATGGCCAGGCCCACTGCTCAGCCCAGCTGTATGTAGAAGAG | |
| | CCCCGGACAGCCGCCTCAGGCCCCAGCTCGAAGCTGGAGAAGATGCCATCCATTCCCG | |
| | AGGAGCCAGAGCAGGGTGAGCTGGAGCGGCTGTCCATTCCTGACTTCCTGCGGCCACT | |
| | GCAGGACCTGGAGGTGGGACTGGCCAAGGAGGCCATGCTAGAGTGCCAGGTGACCGGC | |
| | CTGCCCTACCCCACCATCAGCTGGTTCCACAATGGCCACCGCATCCAGAGCAGCGACG | |
| | ACCGGCGCATGACACAGTACAGGGATGTCCATCGCTTGGTGTTCCCTGCCGTGGGGCC | |
| | TCAGCACGCCGGTGTCTACAAGAGCGTCATTGCCAACAAGCTGGGCAAAAGCTGCCTGC | |
| | TATGCCCACCTGTATGTCACAGATGTGGTCCCAGGCCCTCCAGATGGCGCCCCGCAGG | |
| | TGGTGGCTGTGACGGGGAGGATGGTCACACTCACATGGAACCCCCCCAGGAGTCTGGA | |
| | CATGGCCATCGACCCGGACTCCCTGACGTACACAGTGCAGCACCAGGTGCTGGGCTCG | |
| | GACCAGTGGACGGCACTGGTCACAGGCCTGCGGGAGCCAGGGTGGGCAGCCACAGGGC | |
| | TGCGTAAGGGGGTCCAGCACATCTTCCGGGTCCTCAGCACCACTGTCAAGAGCAGCAG | |
| | CAAGCCCTCACCCCCTTCTGAGCCTGTGCAGCTGCTGGAGCACGGCCCAACCCTGGAG | |
| | GAGGCCCCTGCCATGCTGGACAAACCAGACATCGTGTATGTGGTGGAGGGACAGCCTG | |
| | CCAGCGTCACCGTCACATTCAACCATGTGGAGGCCCAGGTCGTCTGGAGGAGCTGCCG | |
| | AGGGGCCCTCCTAGAGGCACGGGCCGGTGTGTACGAGCTGAGCCAGCCAGATGATGAC | |
| | CAGTACTGTCTTCGGATCTGCCGGGTGAGCCGCCGGGACATGGGGGCCCTCACCTGCA | |
| | CCGCCCGAAACCGTCACGGCACACAGACCTGCTCGGTCACATTGGAGCTGGCAGAGGC | |
| | CCCTCGGTTTGAGTCCATCATGGAGGACGTGGAGGTGGGGGCTGGGGAAACTGCTCGC | |
| | TTTGCGGTGGTGGTCGAGGGAAAACCACTGCCGGACATCATGTGGTACAAGGACGAGG | |
| | TGCTGCTGACCGAGAGCAGCCATGTGAGCTTCGTGTACGAGGAGAATGAGTGCTCCCT | |
| | GGTGGTGCTCAGCACGGGGGCCCAGGATGGAGGCGTCTACACCTGCACCGCCCAGAAC | |
| | CTGGCGGGTGAGGTCTCCTGCAAAGCAGAGTTGGCTGTGCATTCAGCTCAGACAGCTA | |
| | TGGAGGTCGAGGGGGTCGGGGAGGATGAGGACCATCGAGGAAGGAGACTCAGCGACTT | |
| | TTATGACATCCACCAGGAGATCGGCAGGGGTGCTTTCTCCTACTTGCGGCGCATAGTG | |
| | GAGCGTAGCTCCGGCCTGGAGTTTGCGGCCAAGTTCATCCCCAGCCAGGCCAAGCCAA | |
| | AGGCATCAGCGCGTCGGGAGGCCCGGCTGCTGGCCAGGCTCCAGCACGACTGTGTCCT | |
| | CTACTTCCATGAGGCCTTCGAGAGGCGCCGGGGACTGGTCATTGTCACCGAGCTCTGC | |
| | ACAGAGGAGCTGCTGGAGCGAATCGCCAGGAAACCCACCGTGTGTGAGTCTGAGATCC | |
| | GGGCCTATATGCGGCAGGTGCTAGAGGGAATACACTACCTGCACCAGAGCCACGTGCT | |
| | GCACCTCGATGTCAAGCCTGAGAACCTGCTGGTGTGGGATGGTGCTGCGGGCGAGCAG | |
| | CAGGTGCGGATCTGTGACTTTGGGAATGCCCAGGAGCTGACTCCAGGAGAGCCCCAGT | |
| | ACTGCCAGTATGGCACACCTGAGTTTGTAGCACCCGAGATTGTCAATCAGAGCCCCGT | |
| | GTCTGGAGTCACTGACATCTGGCCTGTGGGTCTTGTTGCCTTCCTGCTGTCTGACAGG | |
| | AATCTCCCCGTTTGTTGGGGAAATGACCGGACAACATTGATGAACATCCGAAACTACA | |
| | ACGTGGCCTTCGAGGAGACCACATTCCTGAGCCTGAGCAGGGAGGCCCGGGGCTTCCT | |
| | CATCAAAGTGTTGGTGCAGGACCGGCTGAGACCTACCGCAGAAGAGACCCTAGAACAT | |
| | CCTTGGTTCAAAACTCAGGCAAAGGGCGCAGAGGTGAGCACGGATCACCTGAAGCTAT | |
| | TCCTCTCCCGGCGGAGGTGGCAGCGCTCCCAGATCAGCTACAAATGCCACCTGGTGCT | |
| | GCGCCCCATCCCCGAGCTGCTGCGGGCCCCCCCAGAGCGGGTGTGGGTGACCATGCCC | |
| | AGAAGGCCACCCCCCAGTGGGGGGCTCTCATCCTCCTCGGATTCTGAAGAGGAAGAGC | |
| | TGGAAGAGCTGCCCTCAGTGCCCCGCCCACTGCAGCCCGAGTTCTCTGGCTCCCGGGT | |
| | GTCCCTCACAGACATTCCCACTGAGGATGAGGCCCTGGGGACCCCAGAGACTGGGGCT | |
| | GCCACCCCCATGGACTGGCAGGAGCAGGGAAGGGCTCCCTCTCAGGACCAGGAGGCTC | |
| | CCAGCCCAGAGGCCCTCCCCTCCCCAGGCCAGGAGCCCGCAGCTGGGGCTAGCCCCAG | |
| | GCGGGGAGAGCTCCGCAGGGGCAGCTCGGCTGAGAGCGCCCTGCCCCGGGCCGGGCCG | |
| | CGGGAGCTGGGCCGGGGCCTGCACAAGGCGGCGTCTGTGGAGCTGCCGCAGCGCCGGA | |
| | GCCCCGGCCCGGGAGCCACCCGCCTGGCCCGGGGAGGCCTGGGTGAGGGCGAGTATGC | |
| | CCAGAGGCTGCAGGCCCTGCGCCAGCGGCTGCTGCGGGGAGGCCCCGAGGATGGCAAG | |
| | GTCAGCGGCCTCAGGGGTCCCCTGCTGGAGAGCCTGGGGGGCCGTGCTCGGGACCCCC | |
| | GGATGGCACGAGCTGCCTCCAGCGAGGCAGCGCCCCACCACCAGCCCCCACTCGAGAA | |
| | CCGGGGCCTGCAAAAGAGCAGCAGCTTCTCCCAGGGTGAGGCGGAGCCCCGGGGCCGG | |
| | CACCGCCGAGCGGGGGCGCCCCTCGAGATCCCCGTGGCCAGGCTTGGGGCCCGTAGGC | |
| | TACAGGAGTCTCCTTCCCTGTCTGCCCTCAGCGAGGCCCAGCCATCCAGCCCTGCACG | |
| | GCCCAGCGCCCCCAAACCCAGTACCCCTAAGTCTGCAGAACCTTCTGCCACCACACCT | |
| | AGTGATGCTCCGCAGCCCCCCGCACCCCAGCCTGCCCAAGACAAGGCTCCAGAGCCCA | |
| | GGCCAGAACCAGTCCGAGCCTCCAAGCCTGCACCACCCCCCCAGGCCCTGCAAACCCT | |
| | AGCGCTGCCCCTCACACCCTATGCTCAGATCATTCAGTCCCTCCAGCTGTCAGGCCAC | |
| | GCCCAGGGCCCCTCGCAGGGCCCTGCCGCGCCGCCTTCAGAGCCCAAGCCCCACGCTG | |
| | CTGTCTTTGCCAGGGTGGCCTCCCCACCTCCGGGAGCCCCCGAGAAGCGCGTGCCCTC | |
| | AGCCGGGGGTCCCCCGGTGCTAGCCGAGAAAGCCCGAGTTCCCACGGTGCCCCCCAGG | |
| | CCAGGCAGCAGTCTCAGTAGCAGCATCGAAAACTTGGAGTCGGAGGCCGTGTTCGAGG | |
| | CCAAGTTCAAGCGCAGCCGCGAGTCGCCCCTGTCGCTGGGGCTGCGGCTGCTGAGCCG | |
| | TTCGCGCTCGGAGGAGCGCGGCCCCTTCCGTGGGGCCGAGGAGGAGGATGGCATATAC | |
| | CGGCCCAGCCCGGCGGGGACCCCGCTGGAGCTGGTGCGACGGCCTGAGCGCTCACGCT | |
| | CGGTGCACGACCTCAGGGCTGTCGGAGAGCCTGGCCTCGTCCGCCGCCTCTCGCTGTC | |
| | ACTGTCCCAGCGGCTGCGGCGGACCCCTCCCGCGCAGCGCCACCCGGCCTGGGAGGCC | |
| | CGCGGCGGGGACGGAGAGAGCTCGGAGGGCGGGAGCTCGGCGCGGGGCTCCCCGGTGC | |
| | TGGCGATGCGCAGGCGGCTGAGCTTCACCCTGGAGCGGCTGTCCAGCCGATTGCAGCG | |
| | CAGTGGCAGCAGCGAGGACTCGGGGGGCGCGTCGGGCCGCAGCACGCCGCTGTTCGGA | |
| | CGGCTTCGCAGGGCCACGTCCGAGGGCGAGAGTCTGCGGCGCCTTGGCCTTCCGCACA | |
| | ACCAGTTGGCCGCCCAGGCCGGCGCCACCACGCCTTCCGCCGAGTCCCTGGGCTCCGA | |
| | GGCCAGCGCCACGTCGGGCTCCTCAGCCCCAGGGGAAAGCCGAAGCCGGCTCCGCTGG | |
| | GGCTTCTCTCGGCCGCGGAAGGACAAGGGGTTATCGCCACCAAACCTCTCTGCCAGCG | |
| | TCCAGGAGGAGTTGGGTCACCAGTACGTGCGCAGTGAGTCAGACTTCCCCCCAGTCTT | |
| | CCACATCAAACTCAAGGACCAGGTGCTGCTGGAGGGGGAGGCAGCCACCCTGCTCTGC | |
| | CTGCCAGCGGCCTGCCCTGCACCGCACATCTCCTGGATGAAAGACAAGAAGTCCTTGA | |
| | GGTCAGAGCCCTCAGTGATCATCGTGTCCTGCAAAGATGGGCGGCAGCTGCTCAGCAT | |
| | CCCCCGGGCGGGCAAGCGGCACGCCGGTCTCTATGAGTGCTCGGCCACCAACGTACTG | |
| | GGCAGCATCACCAGCTCCTGTACCGTGGCTGTGGCCCGAGTCCCAGGAAAGCTAGCTC | |
| | CTCCAGAGGTACCCCAGACCTACCAGGACACGGCGCTGGTGCTGTGGAAGCCGGGAGA | |
| | CAGCCGGGCACCTTGCACGTATACGCTGGAGCGGCGAGTGGATGGGGAGTCTGTGTGG | |
| | CACCCTGTGAGCTCAGGCATCCCCGACTGTTACTACAACGTGACCCACCTGCCAGTTG | |
| | GCGTGACTGTGAGGTTCCGTGTGGCCTGTGCCAACCGTGCTGGGCAGGGGCCCTTCAG | |
| | CAACTCTTCTGAGAAGGTCTTTGTCAGGGGTACTCAAGATTCTTCAGCTGTGCCATCT | |
| | GCTGCCCACCAAGAGGCCCCTGTCACCTCAAGGTCAGTCAGGGCCCGGCCTCCTGACT | |
| | CTCCTACCTCACTGGCCTCACCCCTAGCTCCTGCTGCCCCCACACCCCCGTCAGTCAC | |
| | TGTCAGCCCCTCATCTCCCCCCACACCTCCTAGCCAGGCCTTGTCCTCGCTCAAGGCT | |
| | GTGGGTCCACCACCCCAAACCCCTCCACGAAGACACAGGGGCCTGCAGGCTGCCCGGC | |
| | CAGCGGAGCCCACCCTACCCAGTACCCACGTCACCCAAGTGAGCCCCAGCCTTTCGT | |
| | CCTTGACACTGGGACCCCGATCCCAGCCTCCACTCCTCAAGGGGTTAAACCAGTGTCT | |
| | TCCTCTACTCCTGTGTATGTGGTGACTTCCTTTGTGTCTGCACCACCAGCCCCTGAGC | |
| | CCCCAGCCCCTGAGCCCCCTCCTGAGCCTACCAAGGTGACTGTGCAGAGCCTCAGCCC | |
| | GGCCAAGGAGGTGGTCAGCTCCCCTGGGAGCAGTCCCCGAAGCTCTCCCAGGCCTGAG | |
| | GGTACCACTCTTCGACAGGGTCCCCCTCAGAAACCCTACACCTTCCTGGAGGAGAAAG | |
| | CCAGGGGCCGCTTTGGTGTTGTGCGAGCGTGCCGGGAGAATGCCACGGGGCGAACGTT | |
| | CGTGGCCAAGATCGTGCCCTATGCTGCCGAGGGCAAGCCGCGGGTCCTGCAGGAGTAC | |
| | GAGGTGATGCGGACCCTGCACCACGAGCGGATCATGTCCATGCACCAGGCCTACATCA | |
| | CCCCTCGGTACCTCGTGCTCATTGCTGAGAGCTGTGGCAACCGGGAACTCCTCTGTGG | |
| | GCTCAGTGACAGGTTCCGGTATTCTGAGGATGACGTGGCCACTTACATGGTGCAGCTG | |
| | CTACAAGGCCTGGACTACCTCCACGGCCACCACGTGCTCCACCTAGACATCAAGCCAG | |
| | ACAACCTGCTGCTGGCCCCTGACAATGCCCTCAAGATTGTGGACTTTGGCAGTGCCCA | |
| | GCCCTACAACCCCCAGGCCCTTAGGCCCCTTGGCCACCGCACGGGCACGCTGGAGTTC | |
| | ATGGCTCCGGAGATGGTGAAGGGAGAACCCATCGGCTCTGCCACGGACATCTGGGGAG | |
| | CGGGTGTGCTCACTTACATTATGCTCAGTGGACGCTCCCCGTTCTATGAGCCAGACCC | |
| | CCAGGAAACGGAGGCTCGGATTGTGGGGGGCCGCTTTGATGCCTTCCAGCTGTACCCC | |
| | AATACATCCCAGAGCGCCACCCTCTTCTTGCGAAAGGTTCTCTCTGTACATCCCTGGA | |
| | GCCGGCCCTCCCTGCAGGACTGCCTGGCCCACCCATGGTTGCAGGACGCCTACCTGAT | |
| | GAAGCTGCGCCGCCAGACGCTCACCTTCACCACCAACCGGCTCAAGGAGTTCCTGGGC | |
| | GAGCAGCGGCGGCGCCGGGCTGAGGCTGCCACCCGCCACAAGGTGCTGCTGCGCTCCT | |
| | ACCCTGGCGGCCCCTAGAGGCACGGACCACAGCCAGGCCTCGGGCTTCAAAACTGGGGTT | |
| | CCCACCAATGCCACGGGACATTCCAGGGCCCACGCTGAGCCAGGCGGGCCTGGGGCTT | |
| | CGGTTACCACCAGCAGCAACATCTGGCTGGGCTCTTACCTCATAGACCTTCAAGGACA | |
| | GAGACCCCAGGGCCTGGACCTGATGCCACCCCAGGCCAAAGCCAGAGTGGGAGACCCA | |
| | TTGGTCAGGCTCAGCAGGGTGGGAACAGGCAGAGGGACAAGAGGGGAATGGAGAAGTG | |
| | GAGAGGAAGGAATCGAGGGACAGGAAGG | |
| | ORF Start: ATG at 61 | ORF Stop: TAG at 9817 |
| | SEQ ID NO:36 | 3252 aa MW at 352828.6 kD |
| NOV14b, | MQKARGTRGEDAGTRAPPSPGVPPKRAKVGAGGGARVAVAGAPVFLRPLKNAAVCAGS | |
| CG124136-01 | DVRLRVVVSGTPHPILRWFRDGQLLPAPAPEPSCLWLRRCGAQDAGVYSCMAQNERGR | |
| Protein | ASCEAVLTVLEVGDSETAFDDISDVQGTQRLELRDDGAFSTPTGGSDTLVGTSLDTPP | |
| Sequence | TSVTGTSEEQVSWWGSGQTVLEQEAGSGGGTRRLRGSPRQAQATGAGPRHLGVEPLVR | |
| | ASRANLVGASWGSEDSLSVASDLYGSAFSLYRGRALSIHVSVPQSGLRREEPDLQPQL | |
| | ASEAPRRPAQPPPSKSALLPPPSPRVGKRSPPGPPAQPAATPTSPHRRTQEPVLREDT | |
| | TTEEKRGKKSKSSCPSLAGTASSRPQTPLSEASGRLSALORSPRLVRAGSRTLDKLQF | |
| | FEERRRSLERSDSPPAPLRPWVPLRKARSLEQPKSERGAPWGTPGASQEELRAPGSVA | |
| | ERRRLFQQKAASLDERTRQRSPASDLELRPAQSLGRIRRSTSREELVRSHESLRATLQ | |
| | RAPSPREPGEPPLFSRPSTPKTSRAVSPAAAQPPSPSSAEKPGDEPGRPRSRGPAGRT | |
| | EPGEGPQQEVRRRDQFPLTRSRAIQECRSPVPPPAADPPEARTKAPPGRKREPPAQAV | |
| | RFLPWATPGLEGAAVPQTLEKNRAGPSAEKRLRRGPEEDGPWGPWDRRGARSQGKGRR | |
| | ARPTSPELESSDDSYVSAGEEPLEAPVFSIPLQNVVVAPGADVLLKCIITAAPPPQVS | |
| | WHKDGSALRSEGRLLLRAFGERHTLLLREARAADAGSYMATATNELGQATCAASLTVR | |
| | PGGSTSPFSSPTTSDEEYLSPPEEFPEPGETWPRTPTMKPSPSQNRRSSDTGSKAPPT | |
| | FKVSLMDQSVREGQDVIMSIRVQGEPKPVVSWLRNRQPVRPDQRRFAEEAEGGLCRLR | |
| | IILAAERGDAGPYTCKAVNEYGARQCEARLRSEDVDVGAGEMALPECLVAGPTDVEVDW | |
| | LCRGRLLQPALLKCKMHFDGRKCKLLLTSVHEDDSGVYTCKLSTAKDELTCSARLTVR | |
| | PSLAPLFTRLLEDVEVLEGRAARFDCKISGTPPPVVTWTHFGCPMEESENLRLRQDGG | |
| | LHSLHTAHVGSEDEGLYAVSAVNTHGQAHCSAQLYVEEPRTAASGPSSKLEKMPSIPE | |
| | EPEQGELERLSIPDFLRPLQDLEVGLAKEAMLECQVTGLPYPTTSWFHNGHRTQSSDD | |
| | RRMTQYRDVHRLVFPAVGPQHAGVYKSVIANKLGKAACYAHLYVTDVVPGRPDGAPQV | |
| | VAVTGRMVTLTWNPPRSLDMAIDPDSLTYTVQHQVLGSDQWTALVTGLREPGAAATGL | |
| | RKGVQHIFRVLSTTVKSSSKPSPPSEPVQLLEHGPTLEEAPAMLDKPDIVYVVEGQPA | |
| | SVTVTFNHVEAQVVWRSCRGALLEARAGVYELSQRDDDQYCLRICRVSRRDMGALTCT | |
| | ARNRHGTQTCSVTLELAEAPRFESTMEDVEVGAGETARFAVVVEGKPLPDIMWYKDEV | |
| | LLTESSHVSFVYEENECSLVVLSTGAQDGGVYTCTAQNLAGEVSCAAELAVHSAQTAA | |
| | EVEGVGEDEDHRGRRLSDFYDTHQEIGRGAFSYLRRIVERSSGLEPAAKFIPSQAKPK | |
| | ASARREARLLARLQHDCVLYFHEAFERRRGLVIVTELCTEELLERIARKPTVCESETR | |
| | KYMRQVLEGIHYLHQSHVLHLDVKPENLLVWDGAAGEQQVRICDFGNAQELTPGEPQY | |
| | CQYGTPEFVAPEIVNQSPVSGVTDIWPVGVVAFLLSDRNLPVCWGNDRTTLMNIRNYN | |
| | VAFEETTFLSLSREARGPLIKVLVQDRLRPTAEETLEHPWFKTQAKGAEVSTDHLKLF | |
| | LSRRRWQRSQISYKCHLVLRPIPELLRARPERVWVTMPRRPPPSGGLSSSSDSEEEEL | |
| | EELPSVPRPLQPEFSGSRVSLTDIPTEDEALGTPETGAATPMDWQEQGRAPSQDQEAP | |
| | SPEALPSPGQEPAAGASPRRGELRRG8SAESALPRAGPRELGRGLHAAASVELPQRRS | |
| | PGPGATRLARGGLGEGEYAQRLQALRQRLLRGGPEDGKVSGLRGRLLESLGGRARDPR | |
| | MARAASSEAAPHHQPPLENRGLQKSSSFSQGEAEPRGRHRRAGAPLEIPVARLGARRL | |
| | QESPSLSALSEAQPSSPARPSAPKPSTPKSAEPSATTPSDAPQPRARQPAQDAAPEPR | |
| | PEPVRASKPAPPPQALQTLALRLTPYAQIIQSLQLSGHAQGPSQGPAAPPSEPKPHAA | |
| | VPARVASPPPGAPEKRVPSAGGPPVLAEKARVPTVPPRPGSSLSSSIENLESEAVFEA | |
| | KFKRSRESPLSLGLRLLSRSRSEERGPFRGAEEEDGIYRPSPAGTPLELVRRRERSRS | |
| | VQDLRAVGEPGLVRRLSLSLSQRLRRTPRAQRHPAWEARGGDGESSEGGSSARGSPVL | |
| | AMRRRLSFTLERLSSRLQRSGSSEDSGGASGRSTPLFGRLRRATSEGESLRRLGLPHN | |
| | QLAAQAGATTPSAESLGSEASATSGSSARGESRSRLRWGPSRPRKDKGLSPPNLSASV | |
| | QEELGHQYVRSESDFPPVFHIKLKDQVLLEGEAATLLCLRAACPAPHISWMKDKKSLR | |
| | SEPSVIIVSCKDGRQLLSIPRAGKRHAGLYECSATNVLGSITSSCTVAVARVPGKLAP | |
| | REVPQTYQDTALVLWKPGDSPAPCTYTLERRVDGESVWHPVSSGIRDCYYNVTHLPVG | |
| | VTVRFRVACANRAGQGPFSNSSEKVFVRGTQDSSAVPSAAHQEAPVTSRSVRARPPDS | |
| | PTSLASPLAPAAPTPPSVTVSPSSPPTPPSQALSSLKAVGPPPQTPPRRHRGLQAARP | |
| | AEPTLPSTHVTPSEPQPFVLDTGTPIPASTPQGVKPVSSSTPVYVVTSFVSAPPAPEP | |
| | PAREPPPEPTKVTVQSLSPAKEVVSSPGSSPRSSPRPEGTTLRQGPPQKPYTFLEFKA | |
| | RGRPGVVRACRENATGRTFVAKIVPYAAEGKPRVLQEYEVMRTLHHERTMSMHEAYIT | |
| | PRYLVLIAESCGNRELLCGLSDRFRYSEDDVATYMVQLLQGLDYLHGHHVLHLDIKPD | |
| | NLLLAPDNALKIVDFGSAQPYNPQALRPLGHRTGTLEFMAPEMVKGBPIGSATDIWGA | |
| | GVLTYIMLSGRSPFYEPDPQETEARIVGGRFDAFQLYPNTSQSATLFLRKVLSVHPWS | |
| | RPSLQDCLAHPWLQDAYLMKLRRQTLTFTTNRLKSFLGEQRRRRAEAATRHKVLLRSY | |
| | PGGP | |
| | SEQ ID NO:37 | 9698 bp |
| NOV14c, | CCGCGGGTGCCCCCGTGGCCGCCCAGTTCCGGCGTCCCCCCAGCCCAGCTCTCAGTGG | |
| CG124136-03 | CCATGCAGAAAGCCCGGGGCACGCGAGGCGAGGATGCGGGCACGAGGGCACCCCCCAG | |
| DNA | CCCCGGAGTGCCCCCGAAAAAGGGCCAAGGTGGGGGCCGGCGGCGGGGCTCCTGTGGCC | |
| Sequence | GTGGCCGGGGCGCCAGTCTTCCTGCGGCCCCTGAAGAACGCGGCGGTGTGCGCGGGCA | |
| | GCGACGTGCGGCTGCGGGTGGTGGTGAGCGGGACGCCCCAGCCCAGCCTCCGCTGGTT | |
| | CCGGGATGGGCAGCTCCTGCCCGCGCCGGCCCCCGAGCCCAGCTGCCTGTGGCTGCGG | |
| | CGCTGCGGGGCGCAGGACGCCGGCGTGTACAGCTGCATGGCCCAGAACGAGCGGGGCC | |
| | GGGCCTCCTGCGAGGCGGTGCTCACAGTGCTGGAGGTCGGAGACTCAGAGACGGCTGA | |
| | GGATGACATCAGCGATGTGCAGGGAACCCAGCGCCTGGAGCTTCGGGATGACGGGGCC | |
| | TTCAGCACCCCCACGGGGGGTTCTGACACCCTGGTGGGCACCTCCCTGGACACACCCC | |
| | CGACCTCCGTGACAGGCACCTCAGAGGAGCAAGTGAGCTGGTGGGGCAGCGGGCAGAC | |
| | AGCAGCGTCCCTCAGAGCGGGTTGCGCAGGGAGGAGCCCGACCTTCAGCCTCAACTGG | |
| | CCAGCGAAGCCCCACGCCGCCCTGCCCAGCCGCCTCCTTCCAAATCCGCGCTGCTCCC | |
| | CCCACCGTCCCCTCGGGTCGGGAAGCGGTCCCCGCCGGGACCCCCGGCCCAGCCCGCG | |
| | GCCACCCCCACGTCGCCCCACCGTCGCACTCAGGAGCCTGTGCTGCCCGAGGACACCA | |
| | CCACCGAAGAGAAGCGAGGGAAGAAGTCCAAGTCGTCCGGGCCCTCCCTGGCGGGCAC | |
| | CGCGGAATCCCGACCCCAGACGCCACTGAGCGAGGCCTCAGGCCGCCTGTCGGCGTTG | |
| | GGCCGATCGCCTAGGCTGGTGCGCGCCGGCTCCCGCATCCTGGACAAGCTGCAGTTCT | |
| | TCGAGGAGCGACGGCGCAGCCTGGAGCGCAGCGACTCGCCGCCGGCGCCCCTGCGGCC | |
| | CTGGGTGCCCCTGCGCAAGGCCCGCTCTCTGGAGCAGCCCAAGTCGGAGCGCGGCGCA | |
| | CCGTGGGGCACCCCCGGGGCCTCGCAGGAAGAACTGCGGGCGCCAGGCAGCGTGGCCG | |
| | AGCGGCGCCGCCTGTTCCAGCAGAAAGCGGCCTCGCTGGACGAGCGCACGCGTCAGCG | |
| | CAGCCCGGCCTCAGACCTCGAGCTGCGCTTCGCCCAGGAGCTGGGCCGCATCCGCCGC | |
| | TCCACGTCGCGGGAGGAGCTGGTGCGCTCGCACGAGTCCCTGCGCGCCACGCTGCAGC | |
| | GTGCCCCATCCCCTCGAGAGCCCGGCGAGCCCCCGCTCTTCTCTCGGCCCTCCACCCC | |
| | CAAGACATCGCGGGCCGTGAGCCCCGCCGCCGCCCAGCCGCCCTCTCCGAGCAGCGCG | |
| | GAGAAGCCGGGGGACGAGCCTGGGAGGCCCAGGAGCCGCGGGCCGGCGGGCAGGACAG | |
| | AGCCGGGGGAAGGCCCGCAGCAGGAGGTTAGGCGTCGGGACCAATTCCCGCTGACCCG | |
| | GAGCAGAGCCATCCAGGAGTGCAGGAGCCCTGTGCCGCCCCCCGCCGCCGATCCCCCA | |
| | GAGGCCAGGACGAAAGCACCCCCCGGTCGGAAGCGGGAGCCCCCGGCGCAGGCCGTGC | |
| | GCTTCCTGCCCTGGGCCACGCCGGGCCTGGAGGGCGCTGCTGTACCCCAGACCTTGGA | |
| | GAAGAACAGGGCGGGGCCTGAGGCAGAGAAGAGGCTTCGCAGAGGGCCGGAGGAGGAC | |
| | GGTCCCTGGGGGCCCTGGGACCGCCGAGGGGCCCGCAGCCAGGGCAAAGGTCGCCGGG | |
| | CCCGGCCCACCTCCCCTGAGCTCGAGTCTTCGGATGACTCCTACGTGTCCGCTGGAGA | |
| | AGAGCCCCTAGAGGCCCCTGTGTTTGAGATCCCCCTGCAGAATGTGGTGGTGGCACCA | |
| | GGGGCAGATGTGCTGCTCAAGTGTATCATCACTGCCAACCCCCCGCCCCAAGTGTCCT | |
| | GGCACAAGGATGGGTCAGCGCTGCGCAGCGAGGGCCGCCTCCTCCTCCGGGCTGAGGG | |
| | TGAGCGGCACACCCTGCTGCTCAGGGAGGCCAGGGCAGCAGATGCCGGGAGCTATATG | |
| | GCCACCGCCACCAACGAGCTGGGCCAGGCCACCTGTGCCGCCTCACTGACCGTGAGAC | |
| | CCGGTGGGTCTACATCCCCTTTCAGCAGCCCCATCACCTCCGACGAGGAATACCTGAG | |
| | CCCCCCAGAGGAGTTCCCAGAGCCTGGGGAGACCTGGCCGCGAACCCCCACCATGAAG | |
| | CCCAGTCCCAGCCAGAACCGCCGTTCTTCTGACACTGGCTCCAAGGCACCCCCCACCT | |
| | TCAAGGTCTCACTTATGGACCAGTCAGTAAGAGAAGGCCAAGATGTCATCATGAGCAT | |
| | CCGCGTGCAGGGGGAGCCCAAGCCTGTGGTCTCCTGGCTGAGAAACCGCCAGCCCGTG | |
| | CGCCCAGACCAGCGGCGCTTTGCGGAGGAGGCTGAGGGTGGGCTGTGCCGGCTGCGGA | |
| | TCCTGGCTGCAGAGCGTGGCGATGCTGGTTTCTACACTTGCAAAGCGGTCAATGAGTA | |
| | TGGTGCTCGGCAGTGCGAGGCCCGCTTGGAGGTCCGAGCACACCCTGAAAGCCGGTCC | |
| | CTGGCCGTGCTGGCCCCCCTGCAGGACGTGGACGTGGGGGCCGGGGAGATGGCGCTGT | |
| | TTGAGTGCCTGGTGGCGGGGCCCACTGACGTGGAGGTGGATTGGCTGTGCCGTGGCCG | |
| | CCTGCTGCAGCCTGCACTGCTCAATGCAAGATGCATTTCGATGGCCGCAAAAATGCAAG | |
| | CTGCTACTTACATCTGTACATGAGGACGACAGTGGCGTCTACACCTGCAAGCTCAGCA | |
| | CGGCCAAAGATGAGCTGACCTGCAGTGCCCGGCTGACCGTGCGGCCCTCGTTGGCACC | |
| | CCTGTTCACACGGCTGCTGGAAGATGTGGAGGTGTTGGAGGGCCGAGCTGCCCGTTTC | |
| | GACTGCAAGATCAGTGGCACCCCGCCCCCTGTTGTTACCTGGACTCATTTTGGCTGCC | |
| | CCATGGAGGAGAGTGAGAACTTGCGGCTGCGGCAGGACGGGGGTCTGCACTCACTGCA | |
| | CATTGCCCATGTGGGCAGCGAGGACGAGGGGCTCTATGCGGTCAGTGCTGTTAACACC | |
| | CATGGCCAGGCCCACTGCTCAGCCCAGCTGTATGTAGAAGAGCCCCGGACAGCCGCCT | |
| | CAGGCCCCAGCTCGAAGCTGGAGAAGATGCCATCCATTCCCGAGGAGCCAGAGCAGGG | |
| | TGAGCTGGAGCGGCTGTCCATTCCCGACTTCCTGCGGCCACTGCAGGACCTGGAGGTG | |
| | GGACTGGCCAAGGAGGCCATGCTAGAGTGCCAGGTGACCGGCCTGCCCTACCCCACCA | |
| | TCAGCTGGTTCCACAATGGCCACCGCATCCAGAGCAGCGACGACCGGCGCATGACACA | |
| | TACAAGAGCGTCATTGCCAACAAGCTGGGCAAAGCTGCCTGCTATGCCCACCTGTATG | |
| | TCACAGATGTGGTCCCAGGCCCTCCAGATGGCGCCCCGCAGGTGGTGGCTGTGACGGG | |
| | GAGGATGGTCACACTCACATGGAACCCCCCCAGGAGTCTGGACATGGCCATCGACCCG | |
| | GACTCCCTGACGTACACAGTGCAGCACCAGGTGCTGGGCTCGGACCAGTGGACGGCAC | |
| | TGGTCACAGGCCTGCGGGAGCCAGGGTGGGCAGCCACAGGGCTGCGTAAGGGGGTCCA | |
| | GCACATCTTCCGGGTCCTCAGCACCACTGTCAAGAGCAGCAGCAAGCCCTCACCCCCT | |
| | TCTGAGCCTGTGCAGCTGCTGGAGCACGGCCCAACCCTGGAGGAGGCCCCTGCCATGC | |
| | TGGACAAACCAGACATCGTGTATGTGGTGGAGGGACAGCCTGCCAGCGTCACCGTCAC | |
| | ATTCAACCATGTGGAGGCCCAGGTCGTCTGGAGGAGCTGCCGAGGGGCCCTCCTAGAG | |
| | GCACGGGCCGGTGTGTACGAGCTGAGCCAGCCAGATGATGACCAGTACTGTCTTCGGA | |
| | TCTGCCGGGTGAGCCGCCGGGACATGGGGGCCCTCACCTGCACCGCCCGAAACCGTCA | |
| | CGGCACACAGACCTGCTCGGTCACATTGGAGCTGGCAGAGGCCCCTCGGTTTGAGTCC | |
| | ATCATGGAGGACGTGGAGGTGGGGGCTGGGGAAACTGCTCGCTTTGCGGTGGTGGTCG | |
| | AGGGAAAACCACTGCCGGACATCATGTGGTACAAGGACGAGGTGCTGCTGACCGAGAG | |
| | CAGCCATGTGAGCTTCGTGTACGAGGAGAATGAGTGCTCCCTGGTGGTGCTCAGCACG | |
| | GGGGCCCAGGATGGAGGCGTCTACACCTGCACCGCCCAGAACCTGGCGGGTGAGGTCT | |
| | CCTGCAAAGCAGAGTTGGCTGTGCATTCAGCTCAGACAGCTATGGAGGTCGAGGGGGT | |
| | CGGGGAGGATGAGGACCATCGAGGAAGGAGACTCAGCGACTTTTATGACATCCACCAG | |
| | GAGATCGGCAGGGGTGCTTTCTCCTACTTGCGGCGCATAGTGGAGCGTAGCTCCGGCC | |
| | TGGAGTTTGCGGCCAAGTTCATCCCCAGCCAGGCCAAGCCAAAGGCATCAGCGCGTCG | |
| | GGAGGCCCGGCTGCTGGCCAGGCTCCAGCACGACTGTGTCCTCTACTTCCATGAGGCC | |
| | TTCGAGAGGCGCCGGGGACTGGTCATTGTCACCGAGCTCTGCACAGAGGAGCTGCTGG | |
| | AGCGAATCGCCAGGAAACCCACCGTGTGTGAGTCTGAGATCCGGGCCTATATGCGGCA | |
| | GGTGCTAGAGGGAATACACTACCTGCACCAGAGCCACGTGCTGCACCTCGATGTCAAG | |
| | CCTGAGAACCTGCTGGTGTGGGATGGTGCTGCGGGCGAGCAGCAGGTGCGGATCTGTG | |
| | ACTTTGGGAATGCCCAGGAGCTGACTCCAGGAGAGCCCCAGTACTGCCAGTATGGCAC | |
| | ACCTGAGTTTGTAGCACCCGAGATTGTCAATCAGAGCCCCGTGTCTGGAGTCACTGAC | |
| | ATCTGGCCTGTGGGTGTTGTTGCCTTCCTCTGTCTGACAGGAATCTCCCCGTTTGTTG | |
| | GGGAAAAATGACCGGACAACATTGATGAACATCCGAACTACAACGTGGCCTTCGAGGA | |
| | GACCACATTCCTGAGCCTGAGCAGGGAGGCCCGGGGCTTCCTCATCAAAGTGTTGGTG | |
| | CAGGACCGGCTGAGACCTACCGCAGAAGAGACCCTAGAACATCCTTGGTTCAAAACTC | |
| | AGGCAAAGGGCGCAGAGGTGAGCACGGATCACCTGAAGCTATTCCTCTCCCGGCGGAG | |
| | GTGGCAGCGCTCCCAGATCAGCTACAAATGCCACCTGGTGCTGCGCCCCATCCCCGAG | |
| | CTGCTGCGGGCCCCCCCAGAGCGGGTGTGGGTGACCATGCCCAGAAGGCCACCCCCCA | |
| | GTGGGGGGCTCTCATCCTCCTCGGATTCTGAAGAGGAAGAGCTGGAAGAGCTGCCCTC | |
| | AGTGCCCCGCCCACTGCAGCCCGAGTTCTCTGGCTCCCGGGTGTCCCTCACAGACATT | |
| | CCCACTGAGGATGAGGCCCTGGGGACCCCAGAGACTGGGGCTGCCACCCCCATGGACT | |
| | GGCAGGAGCAGGGAAGGGCTCCCTCTCAGGACCAGGAGCCTCCCAGCCCAGAGGCCCT | |
| | CCCCTCCCCAGGCCAGGAGCCCGCAGCTGGGGCTAGCCCCAGGCGGGGAGAGCTCCGC | |
| | ACGGGCAGCTCGGCTGAGAGCGCCCTGCCCCGGGCCGGGCCGCGGGAGCTGGGCCGGG | |
| | GCCTGCACAAGGCGGCGTCTGTGGAGCTGCCGCAGCGCCGGAGCCCCGGCCCGGGAGC | |
| | CACCCGCCTGGCCCGGGCAGGCCTGGGTGAGGGCGAGTATGCCCAGAGGCTGCAGGCC | |
| | CTGCGCCAGCGGCTGCTGCGGGGAGGCCCCGAGGATGGCAAGGTCAGCGGCCTCAGGG | |
| | GTCCCCTGCTGGAGAGCCTGGGGGGCCGTGCTCGGGACCCCCGGATGGCACGAGCTGC | |
| | CTCCAGCGAGGCAGCGCCCCACCACCAGCCCCCACTCGAGAACCGGGGCCTGCAAAAG | |
| | AGCAGCAGCTTCTCCCAGGGTGAGGCGGAGCCCCGGGGCCGGCACCGCCGAGCGGGGG | |
| | CGCCCCTCGAGATCCCCGTGGCCAGGCTTGGGGCCCGTAGGCTACAGGAGTCTCCTTC | |
| | CCTGTCTGCCCTCAGCGAGGCCCAGCCATCCAGCCCTGCACGGCCCAGCGCCCCCAAA | |
| | CCCAGTACCCCTAAGTCTGCAGAACCTTCTGCCACCACACCTAGTGATGCTCCGCAGC | |
| | CCCCCGCACCCCAGCCTGCCCAAGACAAGGCTCCAGAGCCCAGGCCAGAACCAGTCCG | |
| | AGCCTCCAAGCCTGCACCACCCCCCCAGGCCCTGCAAACCCTAGCGCTGCCCCTCACA | |
| | CCCTATGCTCAGATCATTCAGTCCCTCCAGCTGTCAGGCCACGCCCAGGGCCCCTCGC | |
| | AGGGCCCTGCCGCGCCGCCTTCAGAGCCCAAGCCCCACGCTGCTGTCTTTGCCAGGGT | |
| | GGCCTCCCCACCTCCGGGAGCCCCCGAGAAGCGCGTGCCCTCAGCCGGGGGTCCCCCG | |
| | GTGCTAGCCGAGAAAGCCCGAGTTCCCACGGTGCCCCCCAGGCCAGGCAGCAGTCTCA | |
| | GTAGCAGCATCGAAAACTTGGAGTCGGAGGCCGTGTTCGAGGCCAAGTTCAAGCGCAG | |
| | CCGCGAGTCGCCCCTGTCGCTGGGGCTGCGGCTGCTGAGCCGTTCGCGCTCGGAGGAG | |
| | CGCGGCCCCTTCCGTGGGGCCGAGGAGGAGGATGGCATATACCGGCCCAGCCCGGCGG | |
| | GGACCCCGCTGGAGCTGGTGCGACGGCCTGAGCGCTCACGCTCGGTGCAGGACCTCAG | |
| | GGCTGTCGGAGAGCCTGGCCTCGTCCGCCGCCTCTCGCTGTCACTGTCCCAGCGGCTG | |
| | CGGCGGACCCCTCCCGCGCAGCGCCACCCGGCCTGGGAGGCCCGCGGCGGGGACGGAG | |
| | AGAGCTCGGAGGGCGGGAGCTCGGCGCGGGGCTCCCCGGTGCTGGCGATGCGCAGGCG | |
| | GCTGAGCTTCACCCTGGAGCGGCTGTCCAGCCGATTGCAGCCCAGTGGCAGCAGCGAG | |
| | GACTCGGGGGGCGCGTCGGGCCGCAGCACGCCGCTGTTCGGACGGCTTCGCAGGGCCA | |
| | CGTCCGAGGGCGAGAGTCTGCGGCGCCTTGGCCTTCCGCACAACCAGTTGGCCGCCCA | |
| | GGCCGGCGCCACCACGCCTTCCGCCGAGTCCCTGGGCTCCGAGGCCAGCGCCACGTCG | |
| | GGCTCCTCAGCCCCAGGGGAAAGCCGAAGCCGGCTCCGCTGGGGCTTCTCTCGGCCGC | |
| | GGAAGGACAAGGGGTTATCGCCACCAAACCTCTCTGCCAGCGTCCAGGAGGAGTTGGG | |
| | TCACCAGTACGTGCGCAGTGAGTCAGACTTCCCCCCAGTCTTCCACATCAAACTCAAG | |
| | GACCAGGTGCTGCTGGAGGGGGAGGCAGCCACCCTGCTCTGCCTGCCAGCGGCCTGCC | |
| | CTGCACCGCACATCTCCTGGATGAAAGACAAGAAGTCCTTGAGGTCAGAGCCCTCAGT | |
| | GATCATCGTGTCCTGCAAAGATGGGCGGCAGCTGCTCAGCATCCCCCGGGCGGGCAAG | |
| | CGGCACGCCGGTCTCTATGAGTGCTCGGCCACCAACGTACTGGGCAGCATCACCAGCT | |
| | CCTGTACCGTGGCTGTGGCCCGAGTCCCAGGAAAGCTAGCTCCTCCAGAGGTACCCCA | |
| | GACCTACCAGGACACGGCGCTGGTGCTGTGGAAGCCGGGAGACAGCCGGGCACCTTGC | |
| | ACGTATACGCTGGAGCGGCGAGTGGATGGGGAGTCTGTGTGGCACCCTGTGAGCTCAG | |
| | GCATCCCCGACTGTTACTACAACGTGACCCACCTGCCAGTTGGCGTGACTGTGAGGTT | |
| | CCGTGTGGCCTGTGCCAACCGTGCTGGGCAGGGGCCCTTCAGCAACTCTTCTGAGAAG | |
| | GTCTTTGTCAGGGGTACTCAAGATTCTTCAGCTGTGCCATCTGCTGCCCACCAAGAGG | |
| | CCCCTGTCACCTCAAGGCCAGCCAGGGCCCGGCCTCCTGACTCTCCTACCTCACTGGC | |
| | CCCACCCCTAGCTCCTGCTGCCCCCACACCCCCGTCAGTCACTGTCAGCCCCTCATCT | |
| | CCCCCCACACCTCCTAGCCAGGCCTTGTCCTCGCTCAAGGCTGTGGGTCCACCACCCC | |
| | AAACCCCTCCACGAAGACACAGGGGCCTGCAGGCTGCCCGGCCAGCGGAGCCCACCCT | |
| | ACCCAGTACCCACGTCACCCCAAGTGAGCCCAAGCCTTTCGTCCTTGACACTGGGACC | |
| | CCGATCCCAGCCTCCACTCCTCAAGGGGTTAAACCAGTGTCTTCCTCTACTCCTGTGT | |
| | ATGTGGTGACTTCCTTTGTGTCTGCACCACCAGCCCCTGAGCCCCCAGCCCCTGAGCC | |
| | CCCTCCTGAGCCTACCAAGGTGACTGTGCAGAGCCTCAGCCCGGCCAAGGAGGTGGTC | |
| | AGCTCCCCTGGGAGCAGTCCCCGAAGCTCTCCCAGGCCTGAGGGTACCACTCTTCGAC | |
| | AGGGTCCCCCTCAGAAACCCTACACCTTCCTGGAGGAGAAAGCCAGGGGCCGCTTTGG | |
| | TGTTGTGCGAGCGTGCCGGGAGAATGCCACGGGGCGAACGTTCGTGGCCAAGATCGTG | |
| | CCCTATGCTGCCGAGGGCAAGCGGCGGGTCCTGCAGGAGTATGAGGTGCTGCGGACCC | |
| | TGCACCACGAGCGGATCATGTCCCTGCACGAGGCCTACATCACCCCTCGGTACCTCGT | |
| | GCTCATTGCTGAGAGCTGTGGCAACCGGGAACTCCTCTGTGGGCTCAGTGACAGGTTC | |
| | CGGTATTCTGAGGATGACGTGGCCACTTACATGGTGCAGCTGCTACAAGGCCTGGACT | |
| | ACCTCCACGGCCACCACGTGCTCCACCTAGACATCAAGCCAGACAACCTGCTGCTGGC | |
| | CCCTGACAATGCCCTCAAGATTGTGGACTTTGGCAGTGCCCAGCCCTACAACCCCCAG | |
| | GCCCTTAGGCCCCTTGGCCACCGCACGGGCACGCTGGAGTTCATGGCTCCGGAGATGG | |
| | TGAAGGGAGAACCCATCGGCTCTGCCACGGACATCTGGGGAGCGGGTGTGCTCACTTA | |
| | CATTATGCTCAGTGGACGCTCCCCGTTCTATGAGCCAGACCCCCAGGAAACGGAGGCT | |
| | CGGATTGTGGGGGGCCGCTTTGATGCCTTCCAGCTGTACCCCAATACATCCCAGAGCG | |
| | CCACCCTCTTCTTGCGAAAGGTTCTCTCTGTACATCCCTGGAGCCGGCCCTCCCTGCA | |
| | ACGCTCACCTTCACCACCAACCGGCTCAAGGAGTTCCTGGGCGAGCAGCGGCGGCGCC | |
| | GGGCTGAGGCTGCCACCCGCCACAAGGTGCTGCTGCGCTCCTACCCTGGCGGCCCCTA | |
| | GGCGGCCGCTAT | |
| | ORF Start: ATG at 61 | ORF Stop: TAG at 9685 |
| | SEQ ID NO:38 | 3208 aa MW at 348092.4 kD |
| NOV14c, | MQKARGTRGEDAGTRAPPSPGVPPKRAKVGAGGGAPVAVAGAPVFLRPLKNAAVCAGS | |
| CG124136-03 | IDVRLRVVVSGTPQPSLRWFRDGQLLPAPAPEPSCLWLRRCGAQDAGVYSCMAQNERGR | |
| Protein | AAASCEAVLTVLEVGDSETAEDDISDVQGTQRLELRDDGAFSTPTGGSDTLVGTSLDTPP | |
| Sequence | TSVTGTSEEQVSWWGSGQTVLEQEAGSGGGTRRLPGSPSSVPQSGLRREEPDLQPQLA | |
| | SEAPRRPAQPPPSKSALLPPPSPRVGKRSPPGPPAQPAATPTSPHRRTQEPVLPEDTT | |
| | TEEKRGKKSKSSGPSLAGTAESRPQTPLSEASGRLSALGRSPRLVRAGSRILDKLQFE | |
| | EERRRSLERSDSPPAPLRPWVPLRKARSLEQPKSERGAPWGTPGASQEELRAPGSVAE | |
| | RRRLFQQKAASLDERTRQRSPASDLELRFAQELGRIRRSTSREELVRSHESLRATLQR | |
| | APSPREPGEPPLFSRPSTPKTSRAVSPAAAQPPSPSSAEKPGDEPGRPRSRGPAGRTE | |
| | PGEGPQQEVRRRDQFPLTRSRAIQECRSPVPPPAADPPEARTKAPPGRKREPPAQAVR | |
| | FLPWATPGLEGAAVPQTLEKNRAGPEAEKRLRRGPEEDGPWGPWDRRGARSQGKGRRA | |
| | RPTSPELESSDDSYVSAGEEPLEAPVFETPLQNVVVAPGADVLLKCIITANPPPQVSW | |
| | HKDGSALRSEGRLLLRAEGERHTLLLREARAADAGSYMATATNELGQATCAASLTVRP | |
| | GGSTSPFSSPITSDEEYLSPPEEFPEPGETWPRTPTMKPSPSQNRRSSDTGSKAPPTF | |
| | KVSLMDQSVREGQDVIMSTRVQGEPKPVVSWLRNRQPVRPDQRRFAEEAFGGLCRLRT | |
| | LAAERGDAGFYTCKAVNEYGARQCEARLEVRAHPESRSLAVLAPLQDVDVGAGEMALF | |
| | ECLVAGPTDVEVDWLCRGRLLQPALLKCKMHFDGRKCKLLLTSVHEDDSGVYTCKLST | |
| | AKDELTCSARLTVRPSLAPLPTRLLEDVEVLEGRAARPDCKISGTPPPVVTWTHPGCP | |
| | MEESENLRLRQDGGLHSLHIAHVGSEDEGLYAVSAVNTHGQAHCSAQLYVEEPRTAAS | |
| | GPSSKLEKMPSIPEEPEQGELERLSIPDFLRPLQDLEVGLAKEAALECQVTGLPYPTI | |
| | SWFHNGHRIQSSDDRRMTQYRDVHRLVFPAVGPQHAGVYKSVIANKLGKAACYAHLYV | |
| | TDVVPGPPDGAPQVVAVTGRMVTLTWNPPRSLDMAIDPDSLTYTVQHQVLGSDQWTAL | |
| | VTGLREPGWAATGLRKGVQHIFRVLSTTVKSSSKPSPPSEPVQLLEHGPTLEEAPAML | |
| | DKPDIVYVVEGQPASVTVTFNHVEAQVVWRSCRGALLEARAGVYELSQPDDDQYCLRI | |
| | CRVSRRDMGALTCTARNRHGTQTCSVTLELAEAPRFESIMEDVFVGAGETARFAVVVE | |
| | GKPLPDIMWYKDEVLLTESSHVSFVYEENECSLVVLSTGAQDGGVYTCTAQNLAGEVS | |
| | CKAELAVHSAQTAMEVEGVGEDEDHRGRRLSDFYDIHQEIGRGAFSYLRRIVERSSGL | |
| | EFAAKPIPSQAKPKASARREARLLARLQHDCVLYFHEAFERRRGLVIVTELCTEELLE | |
| | RIARKPTVCESEIRAYMRQVLEGIHYLHQSHVLHLDVKPENLLVWDGAAGEQQVRICD | |
| | FGNAQELTPGEPQYCQYGTPEFVAPEIVNQSPVSGVTDIWPVGVVAFLCLTGISPFVG | |
| | ENDRTTLMNIRNYNVAFEETTFLSLSREARGFLIKVLVQDRLRPTAEETLEHPWFKTQ | |
| | AKGAEVSTDHLKLFLSRRRWQRSQTSYKCHLVLRPIPELLRAPPERVWVTMPRRPPPS | |
| | GGLSSSSDSEEEELEELPSVPRPLQPEFSGSRVSLTDIPTEDEALGTPETGAATPMDW | |
| | QEQGRAPSQDQEAPSPEALPSPGQEPAAGASPRRGELRRGSSAESALPRAGPRELGEG | |
| | LHKAASVELPQRRSPGPGATRLARGGLGEGEYAQRLQALRQRLLRGGPEDGKVSGLRG | |
| | PLLESLGGRARDPRMARAASSEAAPHHQPPLENRGLQKSSSFSQGEAEPRGRHRRAGA | |
| | PLEIPVARLGARRLQESPSLSALSEAQPSSPARPSAPKPSTPKSAEPSATTPSDAPQP | |
| | PAPQPAQDKAPEPRPEPVRASKPAPPPQALQTLALPLTPYAQIIQSLQLSGHAQGPSQ | |
| | GPAAPPSEPKPHAAVFARVASPPPGAPEKRVPSAGGPPVLAEKARVPTVPPRPGSSLS | |
| | SSTENLESEAVFEAKEKRSRESPLSLGLRLLSRSRSEERGPERGAEEEDGIYRPSPAG | |
| | TPLELVRRPERSRSVQDLRAVGEPGLVRRLSLSLSQRLRRTPPAQRHPAWEARGGDGE | |
| | SSEGGSSARGSPVLAMRRRLSFTLERLSSRLQRSGSSEDSGGASGRSTPLFGRLRRAT | |
| | SEGESLRRLGLPHNQLAAQAGATTPSAESLGSEASATSGSSAPGESRSRLRWGFSRPR | |
| | KDKGLSPPNLSASVQEELGHQYVRSESDFPPVFHIKLKDQVLLEGEAATLLCLPAACP | |
| | APHISWMKDKKSLRSEPSVIIVSCKDGRQLLSIPRAGKRHAGLYECSATNVLGSITSS | |
| | CTVAVARVPGKLAPPEVPQTYQDTALVLWKPGDSRAPCTYTLERRVDGESVWHPVSSG | |
| | IPDCYYNVTHLPVGVTVRFRVACANRAGQGPFSNSSEKVFVRGTQDSSAVPSAAHQEA | |
| | PVTSRPARARPPDSPTSLAPPLAPAAPTPPSVTVSRSSPPTPPSQALSSLAAVGPPPQ | |
| | TPPRRHRGLQAARPAERTLPSTHVTPSEPKRFVLDTGTPIPASTPQGVKPVSSSTRVY | |
| | VVTSFVSAPPAPEPPAPEPPPERTKVTVQSLSPAKEVVSSPGSSPPSSPRPEGTTLRQ | |
| | GPPQKPYTFLEEKARGRFGVVRACRENATGRTFVAKIVPYAAEGKRRVLQEYEVLRTL | |
| | HHERIMSLHEAYTTPRYLVLIAESCGNRELLCGLSDRPRYSEDDVATYMVQLLQGLDY | |
| | LHGHHVLHLDIKPDNLLLAPDNALKIVDPGSAQPYNPQALRPLGHRTGTLEFMAPEMV | |
| | KGEPIGSATDIWGAGVLTYIMLSGRSPFYEPDPQETEARIVGGRFDAFQLYPNTSQSA | |
| | TLPLRKVLSVHPWSRPSLQDCLAHPWLQDAYLMKLRRQTLTFTTNRLKEFLGEQRRRR | |
| | AEAATRHKVLLPSYPGGP | |
| | SEQ ID NO:39 | 860 bp |
| NOV14d, | ACGGGATCCACCATGGACCATCGAGGAAGGAGACTCAGCGACTTTTATGACATCCACC | |
| 283022671 | AGGAGATCGGCAGGGGTGCTTTCTCCTACTTGCGGCGCATAGTGGAGCGTAGCTCCGG | |
| DNA | CCTGGAGTTTGCGGCCAAGTTCATCCCCAGCCAGGCAAGCCAAGGCATCAGCGCGT | |
| Sequence | CGGGAGGCCCGGCTGCTGGCCAGGCTCCAGCACGACTGTGTCCTCTACTTCCATGAGG | |
| | CCTTCGAGAGGCGCCGGGGACTGGTCATTGTCACCGAGCTCTGCACAGAGGAGCTGCT | |
| | GGAGCGAATCGCCAGGAAACCCACCGTGTGTGAGTCTGAGATCCGGGCCTATATGCGG | |
| | CAGGTGCTAGAGGGAATACACTACCTGCACCAGAGCCACGTGCTGCACCTCGATGTCA | |
| | AGCCTGAGAACCTGCTGGTGTGGGATGGTGCTGCGGGCGAGCAGCAGGTGCGGATCTG | |
| | TGACTTTGGGAATGCCCAGGAGCTGACTCCAGGAGAGCCCCAGTACTGCCAGTATGGC | |
| | ACACCTGAGTTTGTAGCACCCGAGATTGTCAATCAGAGCCCCGTGTCTGGAGTCACTG | |
| | ACATCTGGCCTGTGGGTGTTGTTGCCTTCCTCTGTCTGACAGGAATCTCCCCGTTTGT | |
| | TGGGGAAAATGACCGGACAACATTGATGAACATCCGAAACTACAACGTGGCCTTCGAG | |
| | GAGACCACATTCCTGAGCCTGAGCAGGGAGGCCCGGGGCTTCCTCATCAAAGTGTTGG | |
| | TGCAGGACCGGCTGAGACCTACCGCAGAAGAGACCCTAGAACATCCTTGGTTCAAAAC | |
| | TCAGGCAAAGGGCGCACATCATCACCACCATCACTAGGCGGCCGCAAG | |
| | ORF Start: at 1 | ORF Stop: TAG at 847 |
| | SEQ ID NO:40 | 282 aa MW at 32254.3 kD |

| | |
|---|---|
| NOV14d, | TGSTMDHRGRRLSDFYDIHQEIGRGAFSYLRRIVERSSGLEFAAKFTPSQAKPKASAR |
| 28302267 | REARLLARLQHDCVLYFHEAFERRRGLVIVTELCTEELLERIARKPTVCESEIRAYMR |
| Protein | QVLEGIHYLHQSHVLHLDVKPENLLVWDGAAGEQQVRICDFGNAQELTPGEPQYCQYG |
| Sequence | TPEFVAPEIVNQSPVSGVTDIWRVGVVAFLCLTGISPFVGENDRTTLMNIRNYNVAFF |
| | ETTFLSLSREARGFLIKVLVQDRLRPTAEETLEHPWFKTQAKGAHHHHHH |

Sequence comparison of the above protein sequences yields the following sequence relationships shown in Table 14B.

TABLE 14B
Comparison of NOV14a against NOV14b through NOV14d.

| Protein | NOV14a Residues/ | Identities/Similarities |
|---|---|---|
| Sequence | Match Residues | for the Matched Region |
| NOV14b | 1 . . . 3114 | 2619/3114 (84%) |
| | 1 . . . 3114 | 2623/3114 (84%) |
| NOV14c | 1 . . . 3114 | 2532/3129 (80%) |
| | 1 . . . 3070 | 2536/3129 (80%) |
| NOV14d | 1572 . . . 1846 | 249/275 (90%) |
| | 2 . . . 276 | 249/275 (90%) |

Further analysis of the NOV14a protein yielded the following properties shown in Table 14C.

TABLE 14C
Protein Sequence Properties NOV14a

| | |
|---|---|
| PSort | 0.6000 probability located in endoplasmic reticulum |
| analysis: | (membrane); 0.3500 probability located in nucleus; 0.3000 |
| | probability located in microbody (peroxisome); 0.1000 |
| | probability located in mitochondrial inner membrane |
| SignalP | No Known Signal Sequence Predicted |
| analysis: | |

A search of the NOV14a protein against the Geneseq database, a proprietary database that contains sequences published in patents and patent publication, yielded several homologous proteins shown in Table 14D.

TABLE 14D
Geneseq Results for NOV14a

| | | | Identities/ | |
|---|---|---|---|---|
| | | NOV14a | Similarities | |
| | Protein/ | Residues/ | for the | |
| Geneseq | Organism/Length | Match | Matched | Expect |
| Identifier | [Patent #, Date] | Residues | Region | Value |
| AAE19160 | Human kinase | 960 . . . 3114 | 2133/2155 | 0.0 |
| | polypeptide | | (98%) | |
| | (PKIN-18)— | 88 . . . 2242 | 2137/2155 | |
| | Homo sapiens, | | (98%) | |
| | 2380 aa. | | | |
| | [WO200208399- | | | |
| | A2, 31 JAN. | | | |
| | 2002] | | | |
| AAB65635 | Novel protein | 967 . . . 3114 | 2127/2148 | 0.0 |
| | kinase, SEQ ID | | (99%) | |
| | NO: 162—Homo | 1 . . . 2148 | 2130/2148 | |
| | sapiens, 2286 aa | | (99%) | |
| | [WO200073469- | | | |
| | A2, 7 DEC. | | | |
| | 2000] | | | |
| AAY70078 | Human striated | 321 . . . 957 | 600/645 | 0.0 |
| | muscle | | (93%) | |
| | preferentially | 13 . . . 656 | 602/645 | |
| | expressed partial | | (93%) | |
| | protein—Homo | | | |
| | sapiens, 661 aa. | | | |
| | [WO200009689- | | | |
| | A2, 24 FEB. | | | |
| | 2000] | | | |
| AAW77048 | Human striated | 321 . . . 957 | 600/645 | 0.0 |
| | muscle | | (93%) | |
| | preferentially | 13 . . . 656 | 602/645 | |
| | expressed | | (93%) | |
| | protein—Homo | | | |
| | sapiens, 661 aa. | | | |
| | [WO9835040-A2, | | | |
| | 13 AUG. 1998] | | | |
| AAY70079 | Mouse striated | 365 . . . 957 | 553/594 | 0.0 |
| | muscle | | (93%) | |
| | preferentially | 4 . . . 597 | 566/594 | |
| | expressed partial | | (95%) | |
| | protein—Mus sp, | | | |
| | 602 aa. | | | |
| | [WO200009689- | | | |
| | A2, 24 FEB. | | | |
| | 2000] | | | |

In a BLAST search of public sequence databases, the NOV14a protein was found to have homology to the proteins shown in the BLASTP data in Table 14E.

TABLE 14E
Public BLASTP Results for NOV14a

| | | | Identities/ | |
|---|---|---|---|---|
| | | NOV14a | Similarities | |
| Protein | | Residues/ | for the | |
| Accession | Protein/ | Match | Matched | Expect |
| Number | Organism/Length | Residues | Portion | Value |
| Q9EQJ5 | Striated muscle- | 1 . . . 3114 | 2776/3134 | 0.0 |
| | specific serine/ | | (88%) | |
| | threonine protein | 1 . . . 3124 | 2865/3134 | |
| | kinase—Mus | | (90%) | |
| | musculus | | | |
| | (Mouse), 3262 aa. | | | |
| Q9P2P9 | KIAA1297 | 967 . . . 3114 | 2127/2148 | 0.0 |
| | protein—Homo | | (99%) | |
| | sapiens (Human), | 1 . . . 2148 | 2130/2148 | |
| | 2242 aa | | (99%) | |
| | (fragment). | | | |
| CAC16626 | Sequence 5 from | 1027 . . . 2315 | 388/1328 | e−123 |
| | Patent | | (29%) | |
| | WO0063381— | 620 . . . 1784 | 569/1328 | |
| | Homo sapiens | | (42%) | |
| | (Human), | | | |
| | 2596 aa. | | | |
| CAC16625 | Sequence 3 from | 1476 . . . 2315 | 299/873 | e−112 |
| | Patent | | (34%) | |
| | WO0063381— | 5 . . . 798 | 418/873 | |
| | Homo sapiens | | (47%) | |
| | (Human), | | | |
| | 1610 aa. | | | |
| Q15772 | Aortic | 850 . . . 957 | 108/108 | 4e−56 |
| | preferentially | | (100%) | |
| | expressed protein | 1 . . . 108 | 108/108 | |
| | 1 (APEG-1)— | | (100%) | |
| | Homo sapiens | | | |
| | (Human), 113 aa. | | | |

PFam analysis predicts that the NOV14a protein contains the domains shown in the Table 14F.

TABLE 14F
Domain Analysis of NOV14a

| Pfam | NOV14a | Identities/Similarities | Expect |
|---|---|---|---|
| Domain | Match Region | for the Matched Region | Value |
| Ig | 57 . . . 110 | 16/57 (28%) | 0.00069 |
| | | 40/57 (70%) | |
| Ig | 736 . . . 796 | 13/64 (20%) | 1.1e−06 |
| | | 43/64 (67%) | |
| Ig | 883 . . . 944 | 18/65 (28%) | 5.9e−07 |
| | | 47/65 (72%) | |
| Ig | 967 . . . 1028 | 19/65 (29%) | 1.2e−07 |
| | | 40/65 (62%) | |
| Ig | 1063 . . . 1119 | 17/60 (28%) | 0.012 |
| | | 39/60 (65%) | |
| Ig | 1187 . . . 1243 | 14/60 (23%) | 0.0018 |
| | | 41/60 (68%) | |
| Fn3 | 1267 . . . 1356 | 22/91 (24%) | 0.00055 |
| | | 55/91 (60%) | |
| Ig | 1484 . . . 1544 | 15/64 (23%) | 8e−05 |
| | | 39/64 (61%) | |
| Rhabd_nucleocap | 1587 . . . 1608 | 6/22 (27%) | 0.75 |
| | | 18/22 (82%) | |
| Pkinase | 1586 . . . 1839 | 73/297 (25%) | |
| | | 181/297 (61%) | 2.9e−47 |
| Ig | 2583 . . . 2644 | 14/65 (22%) | 9.6e−06 |
| | | 40/65 (62%) | |
| Fn3 | 2663 . . . 2745 | 20/87 (23%) | 0.064 |
| | | 51/87 (59%) | |
| Pkinase | 2951 . . . 3092 | 47/143 (33%) | 1.8e−37 |
| | | 110/143 (77%) | |

### Example 15

The NOV15 clone was analyzed and the neucleoticle and encoded polypeptide sequences are shorn in Table 15A.

TABLE 15A
NOV15 Sequence Analysis

| | SEQ ID NO:41 | 3109 bp |
|---|---|---|
| NOV15a, | AGGGGCTGAGGAGGTACTGGAAPAGAAAAAGAGGAGCAGGAGCTGGAGGAAGACGTGGAG | |
| CG124553-01 | GAGGAGCTGGAGGAGGATGAAGAGAAGGAGTGGGACGCCCACPACCCTGTGTAAGGAG | |
| DNA | CTCAAGTACTCCAAGGACCCGCCCCAGATATCCATCATATTCATCTTCGTGAACGAGG | |
| Sequence | CCCTGTCGGTGATCCTGCGGTCCGTGCACAGTGCCGTCAATCACACGCCCACACACCT | |
| | GCTGAAGGAAATCATTCTGGTGGATGACAACAGCGACGAAGAGGAGCTGAAGGTCCCC | |
| | CTAGAGGAGTATGTCCACAAACGCTACCCCGGGCTGGTGAAGGTGGTAAGAAATCAGA | |
| | AGAGGGAAGGCCTGATCCGCGCTCGCATTGAGGGCTGGAAGGTGGCTACCGGGCAGGT | |
| | CACTGGCTTCTTTGATGCCCACGTGGAATTCACCGCTGGCTGGGCTGAGCCGGTTCTA | |
| | TCCCGCATCCAGGAAAACCGGAAGCGTGTGATCCTCCCCTCCATTGACAACATCAAAC | |
| | AGGACAACTTTGAGGTGCAGCGGTACGAGAACTCGGCCCACGGGTACAGCTGGGAGCT | |
| | GTGGTGCATGTACATCAGCCCCCCAAAAGACTGGTGGGACGCCGGAGACCCTTCTCTC | |
| | CCCATCAGGACCCCAGCCATGATAGGCTGCTCGTTCGTGGTCAACAGGAAGTTCTTCG | |
| | GTGAAATTGGTCTTCTGGATCCTGGCATGGATGTATACGGAGGAGAAAATATTGAACT | |
| | GGGAATCAAGGTATGGCTCTGTGGGGGCAGCATGGAGGTCCTTCCTTGCTCACGGGTG | |
| | GCCCACATTGAGCGGAAGAAGAAGCCATATAATAGCAACATTGGCTTCTACACCAAGA | |
| | GGAATGCTCTTCGCGTTGCTGAGGTCTGGATGGACGATTACAAGTCTCATGTGTACAT | |
| | AGCGTGGAACCTGCCGCTGGAGAATCCGGGAATTGACATCGGTGATGTCTCCGAAAGA | |
| | AGAGCATTAAGGAAAAGTTTAAAGTGTAAGAATTTCCAGTGGTACCTGGACCATGTTT | |
| | ACCCAGAAATGAGAAGATACAATAATACCGTTGCTTACGGGGAGCTTCGCAACAACAAA | |
| | GGCAAAAGACGTCTGCTTGGACCAGGGGCCGCTGGAGAACCACACAGCAATATTGTAT | |
| | CCGTGCCATGGCTGGGGACCACAGCTTGCCCGCTACACCAAGGAAGGCTTCCTGCACT | |
| | TGGGTGCCCTGGGGACCACCACACTCCTCCCTGACACCCGCTGCCTGGTGGACAACTC | |
| | CAAGAGTCGGCTGCCCCAGCTCCTGGACTGCGACAAGGTCAAGAGCAGCCTGTACAAG | |
| | CGCTGGAACTTCATCCAGAATGGAGCCATCATGAACAAGGGCACGGGACGCTGCCTGG | |
| | AGGTGGAGAACCGGGGCCTGGCTGGCATCGACCTCATCCTCCGCAGCTGCACAGGTCA | |
| | GAGGTGGACCATTAAGAACTCCATCAAGTAGAGGGAGGGAGCTGGGGCACTGGAGCCT | |
| | GGCCCCCAGGACATGGCTGCTCCCCCCAACATCTGGACCAGCTGCCCTGGCGGAGAGA | |
| | CAGCAAGGGGCCGGCAGGTGCTCGATGGGCCCCCCAGGGCTTCTCCAGGGCAGCACAG | |
| | GGACCCCGGATGAAGACTCTGTCCCCCCTCAGGCATTCAGCTGCCCACAAGTTTCCTG | |
| | CACCCTGGAAAAGCCCCCCACCCTTCCTCTGGGAAACTGACAGCTGTCTTCCACAGCC | |
| | TCTGATGTGGACCTGGTACTGAGGAGCAAGACTGTCCAGTTCTCCTCCACATCTCCCA | |
| | TCCCAGAATCAGGATCTGGGACTGGCAGGGTCCCCTCCTGTGTCTCATCTCTTGCAGC | |
| | AGCAGCTGCTGAACTCCAGCCATCAACACGGTGGGAGGCAGCGGGGGCTTCAGCCATG | |
| | TCCTAGCTCCCCGCCCTAAAAGGAGGCAGTGAGGACCAGGCACTATTTCCTCCGAGGT | |
| | TACTTCTACCCAGATGACACCTGCCTGTTCACGCCCCAAGGCAGCTACTGCCCCTAAC | |
| | CCTTCCCACCAGGGTAGCTTTGGGCACTGCAGCTCTGGACTTTTCTGGCCCCTCCTGA | |
| | GATGACCTGATGGAGCTGATGCTTTCTCTCCTAATCCCTGGGCACTAGGCTCTTATCA | |
| | GTGTGCTTGGGCCAGCTCTCCTGCCTGTGTCTAGAGGAAGCCAGAGACAGAAATAGGC | |
| | TAAGCCTGCAGTAGGATCTCAGCCACAAGGGCCCCGCAGGATGGAGCTGGGTCAAGGA | |
| | CCAGGGAGCCCTGACTCCCAGAGGCTGCCACCGGGGAGAAGCAGCGGTCCTCCATCCA | |
| | GAACCTAAGGGCTGAAGCAAAGGCTGCCAGGACCCTTGAAGATGCTTTTGGCTCACCT | |
| | CATTTCACCCCACGCTCTGCTGGCTGGCAGAGGAGAAGGCAGTCGTTTCCTCTCTGAA | |
| | GAGTATTTTTTTCGATTGCCCTCTGGTTAGGGTGCACATATAAATCAGAGTTAATATA | |
| | TGAACGCGTGTGCATGCACAAGTGTGTGTGTGCCTGCGTGCTGTGCGTGGCAGGGTGT | |
| | GTGTGTGTGTGTCTGGCTGTGCGTTCCGGAGTGTGTGACGATGCTGACCTAGCTGTGT | |
| | GGCCTTGGGCTTGCTGCTTCATTACTCACCTGGATGGGGACGAGGGATGAGAAGGGTG | |
| | TGGGTTTGGCCCCATGTCACTGGCCGGAAGGATGTGTCTCAGCCCTGCCCTGTGGGGT | |
| | GCCCCCGATGGGAGGCTGTCCCATCTCCCAGTCCCCATCTCTTTTTCCCCACACTGTC | |
| | CCTGGCCAAGCCCTGCCCAGAGCTGAACCCTGTAGCTGCCCCCTTGCCCTGTGTGGGA | |
| | TTCGCAGTGTCTCATTTGGTGACGTCTTACTGGTGATCATCTCCTCACCCCATCTCCC | |
| | ACCTTGTGGAATAAATACATGTTAGCACTTCCCAGAGCAGCCTCCTTTGTGTCTTGAT | |
| | TTCTCCAGAACTGGAGGTGGGGAGGGGAGTGATGGAGACATAGGAGGAGAGCTTCTTT | |
| | GGCTTTGAGGGTTTAGTGTTACTTATTTATCTATTTATTCGAGATGGGGTCTTGCTCT | |
| | GTGGCCCAGGCTGGAGTGCAGTGGTGCAATCATGA | |
| | ORF Start: ATG at 75 | ORF Stop: TAG at 1479 |
| | SEQ ID NO:42 | 468 aa MW at 53596.1 kD |
| NOV15a, | MKRRSGTPTTLCKELKYSKDPPQISIIFIPVNEALSVILRSVHSAVNHTPTHLLKEII | |
| CG124553-01 | LVDDNSDEEELKVPLEEYVHKRYRGLVKVVRNQKREGLIRARTEGWKVATGQVTGFFD | |
| Protein | AHVEFTAGWAEPVLSRIQENRKRVILPSIDNIKQDNFEVQRYENSAHGYSWELWCMYI | |
| Sequence | SPPKDWWDAGDPSLPIRTPAMIGCSFVVNRKPFGEIGLLDPGMDVYGGENIELGIKVW | |
| | LCGGSMEVLPCSRVAHIERKKKRYNSNTGPYTKRNALRVAEVWMDDYKSHVYIAWNLP | |
| | LENPGIDIGDVSERRALRKSLKCKNFQWYLDHVYPEMRRYNNTVAYGELRNNKAKDVC | |
| | LDQGPLFNHTAILYPCHGWGPQLARYTKEGFLHLGALGTTTLLPDTRCLVDNSKSRLP | |
| | QLLDCDKVKSSLYKRWNFIQNGAIMNKGTGRCLEVENRGLAGIDLILRSCTGQRWTIK | |
| | NSIK | |
| | SEQ ID NO:43 | 580 bp |
| NOV15b, | CACCAGATCTTCCATCATATTCATCTTCGTGAACGAGGCCCTGTCGGTGATCCTGCGG | |
| 276644723 | TCCGTGCACAGTGCCGTCAATCACACGCCCACACACCTGCTGAAGGAAAAATCATTCTGG | |
| DNA | TGGATGACAAACAGCGACGAAGAGGAGCTGAAGGTCCCCCTAGAGGAGTATGTCCACAAA | |
| Sequence | ACGCTACCCCGGGCTGGTGAAGGTGGTAAGAATCAGAAGAGGGAAGGCCTGATCCGC | |
| | GCTCGCATTGAGGGCTGGAAGGTGGCTACCGGGCAGGTCACTGGCTTCTTTGATGCCC | |
| | ACGTGGAATTCACCGCTGGCTGGGCTGAGCCGGTTCTATCCCGCATCCAGGAAAACCG | |
| | GAAGCGTGTGATCCTCCCCTCCATTGACAACATCAAACAGGACAACTTTGAGGTGCAG | |
| | CGGTACGAGAACTCGGCCCACGGGTACAGCTGGGAGCTGTGGTGCATGTACATCAGCC | |
| | CCCCAAAAGACTGGTGGGACGCCGGAGACCCTTCTCTCCCCATCAGGACCCCAGCCAT | |
| | GATAGGCTGCTCGTTCGTGGTCAACAGGAAGTTCTTCGGTGAAATTGGTCTCGAGGGC | |
| | ORF Start: at 2 | ORF Stop: end of sequence |
| | SEQ ID NO:44 | 193 aa MW at 22163.1 kD |
| NOV15b, | TRSSIIFTFVNEALSVILRSVHSAVNHTPTHLLKEIILVDDNSDEEELKVPLEEYVHK | |
| 276644723 | RYPGLVKVVRNQKREGLTRARIEGWKVATGQVTGFFDAHVEFTAGWAEPVLSRIQENR | |
| Protein | KRVILPSIDNIKQDNFEVQRYENSAHGYSWELWCMYISPPKDWWDAGDPSLPIRTPAM | |
| Sequence | IGCSFVVNRKPFGEIGLEG | |
| | SEQ ID NO:45 | 495 bp |
| NOV15c, | CACCAGATCTTCCATCATATTCATCTTCGTGAACGAGGCCCTGTCGGTGATCCTGCGG | |
| 276644750 | TCCGTGCACAGTGCCGTCAATCACACGCCCACACACCTGCTGAAGGAAATCATTCTGG | |
| DNA | TGGATGACAACAGCGACGAAGAGGAGCTGAAGGTCCCCCTAGAGGAGTATGTCCACAA | |
| Sequence | ACGCTACCCCGGGCTGGTGAAGGTGGTAAGAAATCAGAAGAGGGAAGGCCTGATCCGC | |
| | GCTCGCATTGAGGGCTGGAAGGTGGCTACCGGGCAGGTCACTGGCTTCTTTGATGCCC | |
| | ACGTGGAATTCACCGCTGGCTGGGCTGAGCCGGTTCTATCCCGCATCCAGGAAAACCG | |
| | GAAGCGTGTGATCCTCCCCTCCATTGACAACATCAAACAGGACAACTTTGAGGTGCAG | |
| | CGGACCCCAGCCATGATAGGCTGCTCGTTCGTGGTCAACAGGAAGTTCTTCGGTGAAA | |
| | TTGGTCTCGAGGGCAAGGGCGAATTCCAGCA | |
| | ORF Start: at 2 | ORF Stop: at 494 |
| | SEQ ID NO:46 | 164 aa MW at 18699.3 kD |

| | |
|---|---|
| NOV15c, | TRSSITFIFVNEALSVILRSVHSAVNHTPTHLLKEIILVDDNSDEEFLKVPLEEYVHK |
| 276644750 | RYPGLVKVVRNQKREGLIRARIFGWKVATGQVTGFFDAHVFFTAGWAERVLSRTQENR |
| Protein | KRVILPSIDNIKQDNFEVQRTRANIGCSFVVNRKFFGEIGLFGKGEPQ |
| Sequence | |

sequence comparison of the above protein sequences yields the following sequence relationships shown in Table 15B.

TABLE 15B
Comparison of NOV15a against NOV15b and NOV15c.

| Protein | NOV15a Residues/ | Identities/Similarities |
|---|---|---|
| Sequence | Match Residues | for the Matched Region |
| NOV15b | 25 . . . 212 | 188/188 (100%) |
| | 4 . . . 191 | 188/188 (100%) |
| NOV15c | 25 . . . 216 | 155/192 (80%) |
| | 4 . . . 161 | 155/192 (80%) |

Further analysis of the NOV15a protein yielded the following properties shown in Table 15C.

TABLE 15C
Protein Sequence Properties NOV15a

| | |
|---|---|
| PSort | 0.7900 probability located in plasma membrane; 0.3488 |
| analysis: | probability located in microbody (peroxisome); 0.3000 |
| | probability located in Golgi body; 0.3000 probability located |
| | in nucleus |
| SignalP | No Known Signal Sequence Predicted |
| analysis: | |

A search of the NOV15a protein against the Geneseq database, a proprietary database that contains sequences published in patents and patent publication, yielded several homologous proteins shown in Table 15D.

TABLE 15D
Geneseq Results for NOV15a

| | | | Identities/ | |
|---|---|---|---|---|
| | | NOV15a | Similarities | |
| | Protein/ | Residues/ | for the | |
| Geneseq | Organism/Length | Match | Matched | Expect |
| Identifier | [Patent #, Date] | Residues | Region | Value |
| AAM41675 | Human polypeptide | 10 . . . 468 | 457/459 | 0.0 |
| | SEQ ID NO 6606— | | (99%) | |
| | Homo sapiens, | 102 . . . 560 | 457/459 | |
| | 560 aa. | | (99%) | |
| | [WO200153312-A1, | | | |
| | 26 JUL. 2001] | | | |
| AAM40865 | Human polypeptide | 167 . . . 468 | 302/302 | 0.0 |
| | SEQ ID NO 5796— | | (100%) | |
| | Homo sapiens, | 5 . . . 306 | 302/302 | |
| | 358 aa. | | (100%) | |
| | [WO200153312-A1, | | | |
| | 26 JUL. 2001] | | | |
| AAM39079 | Human polypeptide | 172 . . . 468 | 296/297 | 0.0 |
| | SEQ ID NO 2224— | | (99%) | |
| | Homo sapiens, | 1 . . . 297 | 296/297 | |
| | 297 aa. | | (99%) | |
| | [WO200153312-A1, | | | |
| | 26 JUL. 2001] | | | |
| AAM40398 | Human polypeptide | 101 . . . 468 | 237/370 | e−152 |
| | SEQ ID NO 3543— | | (64%) | |
| | Homo sapiens, | 29 . . . 398 | 298/370 | |
| | 402 aa. | | (80%) | |
| | [WO200153312-A1, | | | |
| | 26 JUL. 2001] | | | |
| AAM42184 | Human polypeptide | 160 . . . 468 | 198/311 | e−125 |
| | SEQ ID NO 7115— | | (63%) | |
| | Homo sapiens, | 1 . . . 311 | 250/311 | |
| | 315 aa. | | (79%) | |
| | [WO200153312-A1, | | | |
| | 26 JUL. 2001] | | | |

In a BLAST search of public sequence databases, the NOV15a protein as found to have homology to the proteins shown in the BLASTP data in Table 5E.

TABLE 15E
Public BLASTP Results for NOV15a

| | | NOV15a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| AAM62306 | Putative poly- | 10 . . . 468 | 457/459 (99%) | 0.0 |
| | peptide N-acetyl- | 140 . . . 598 | 457/459 (99%) | |
| | galactosaminyl- | | | |
| | transferase— | | | |
| | Homo sapiens | | | |
| | (Human), 598 aa. | | | |
| AAM62404 | Williams-Beuren | 10 . . . 468 | 447/459 (97%) | 0.0 |
| | syndrome critical | 140 . . . 596 | 450/459 (97%) | |
| | region gene 17— | | | |
| | Mus musculus | | | |
| | (Mouse), 596 aa. | | | |
| Q9GM01 | UDP-GalNAc: | 12 . . . 468 | 303/459 (66%) | 0.0 |
| | polypeptide N- | 144 . . . 602 | 377/459 (82%) | |
| | acetylgalactos- | | | |
| | aminyl- | | | |
| | transferase— | | | |
| | Macaca | | | |
| | fascicularis (Crab | | | |
| | eating macaque) | | | |
| | (Cynomolgus | | | |
| | monkey), 606 aa. | | | |
| Q9HCQ5 | UDP-GalNAc: | 12 . . . 468 | 302/459 (65%) | 0.0 |
| | polypeptide N- | 141 . . . 599 | 377/459 (81%) | |
| | acetylgalactos- | | | |
| | aminyl- | | | |
| | transferase— | | | |
| | Homo sapiens | | | |
| | (Human), 603 aa. | | | |
| Q9NY28 | UDP-N-acetyl- | 12 . . . 468 | 219/461 (47%) | e−129 |
| | alpha-D-galactos- | 171 . . . 629 | 310/461 (66%) | |
| | amine:poly- | | | |
| | peptide N-acetyl- | | | |
| | galactosaminyl- | | | |
| | transferase 8— | | | |
| | Homo sapiens | | | |
| | (Human), 637 aa. | | | |

PFam analysis predicts that the NOV15a protein contains the domains shown in the Table 15F.

TABLE 15F
Domain Analysis of NOV15a

| Pfam | NOV15a | Identities/Similarities | Expect |
|---|---|---|---|
| Domain | Match Region | for the Matched Region | Value |
| Glycos_transf_2 | 25 . . . 211 | 45/189 (24%) | 2.4e−31 |
| | | 143/189 (76%) | |
| Ricin_B_lectin | 428 . . . 466 | 13/47 (28%) | 0.14 |
| | | 29/47 (62%) | |

### Example 16

The NOV16 clone was analyzed, and the nucleotide and encoded polypeptide sequences are shown in Table 16A.

TABLE 16A
NOV16 Sequence Analysis

| | |
|---|---|
| SEQ ID NO: 47 | 2422 bp |

| | |
|---|---|
| NOV16a, | CGCCAAGGCAGCCGGCGCTGGCGATGGGAAGCGGCGTGGCCGCCGACACAGGCAGTGG |
| CG124691- | |
| 01 DNA | CAAAGTTTCCCAGACGTACACATCTGGACGCGCGGCTGCCGGCTACCCGTGACCCCTC |
| Sequence | |
| | TAGGAAGGGTTCAGGGATTTTTAATTTGGAAAAAAATCCACCTGGTTTCCTTTGTCAA |
| | GGTCTCTCCGGGTGGCCAGCGGCAGGAGCTGCAAACTTGGGCACGGCGGCTACACCGG |
| | CAGCGGACCGGGCTTTGGAGAACCTCGGGACTCAGGTGCTGAGGTGCCCAGCGGCTCC |
| | GGACGTGCTACGGGGTGCGAGCGCGGGGGAGTTCGGGGCGCACGACAAGGAAGGGCCC |
| | CCGGGAGCTCTATATGGAGGAAGGAGCCCAGAATGGTGTGCACCAGGAAGACCAAAAC |
| | TTTGGTGTCCACTTGCGTGATCCTGAGCGGCATGACTAACATCATCTGCCTGCTCTAC |
| | GTGGGCTGGGTCACCAACTACATCGCCAGCGTGTATGTGCGGGGGCAGGAGCCGGCGC |
| | CCGACAAGAAGCTGGAGGAAGACAAAGGGGACACTCTGAAGATTATTGAGCGGCTGGA |
| | CCACCTGGAGAATGTCATCAAGCAGCACATTCAAGAGGCTCCTGCCAAGCCTGAGGAG |
| | GCAGAGGCCGAGCCCTTCACAGACTCCTCTCTGTTTGCACACTGGGGCCAGGAGCTCA |
| | GCCCCGAAGGCCGGCGCGTGGCCCTGAAGCAATTCCAGTACTACGGCTACAACGCCTA |
| | CCTCAGCGACCGCCTGCCCCTGGACCGGCCCCTGCCTGACCTCAGACCCAGTGGGTGC |
| | CGTAACCTCTCATTTCCTGACAGCCTGCCAGAGGTGAGCATCGTGTTCATCTTCGTCA |
| | ATGAAGCGCTTTCAGTGCTGCTGCGCTCCATCCACTCGGCCATGGAACGCACGCCCCC |
| | ACATCTGCTCAAGGAGATCATTCTGGTGGATGACAACAGCAGTAACGAGGAACTGAAG |
| | GAGAAGCTGACCGAATATGTGGACAAGGTGAACAGCCAGAAGCCAGGCTTCATCAAAG |
| | TCGTGCGTCACAGCAAGCAGGAAGGCCTCATCCGCTCCAGGGTCAGTGGCTGGAGGGC |
| | GGCCACTGCCCCTGTGGTGGCACTCTTTGATGCCCACGTGGAGTTCAATGTGGGCTGG |
| | GCTGAACCTGTACTCACCCGCATCAAGGAGAACCGGAAGCGGATCATCTCGCCATCCT |
| | TTGATAACATCAAATATGACAACTTTGAGATAGAAGAGTACCCGCTGGCTGCCCAGGG |
| | CTTTGACTGGGAGCTGTGGTGCCGCTACCTAAATCCCCCCAAGGCCTGGTGGAAGCTG |
| | GAGAACTCCACAGCGCCAATCAGGAGCCCTGCCCTCATTGGCTGCTTCATTGTGGACC |
| | GGCAGTACTTCCAGGAGATCGGCCTGCTGGACGAAGGCATGGAAGTCTACGGGGGCGA |
| | GAATGTGGAGCTTGGGATCAGGGTGTGGCAGTGTGGCGGGAGTGTGGAGGTCCTGCCC |
| | TGCTCACGGATTGCCCACATTGAGCGAGCCCACAAGCCCTACACAGAGGACCTCACCG |
| | CCCATGTCCGCAGGAACGCTCTCAGGGTGGCTGAAGTCTGGATGGATGAATTTAAAAG |
| | CCACGTCTACATGGCATGGAACATACCGCAGGAGGACTCAGGAATTGACATGGGGGAC |
| | ATCACGGCAAGGAAGGCTCTCAGGAAACAGCTGCAGTGCAAGACCTTCCGGTGGTACC |
| | TGGTCAGCGTGTACCCAGAGATGAGGATGTACTCCGACATCATTGCCTATGGAGTGCT |
| | GCAGAATTCTCTGAAGACTGATTTGTGTCTTGACCAGGGGCCAGATACAGAGAATGTC |
| | CCCATCATGTACATCTGCCATGGGATGACGCCTCAGAACGTGTACTACACGAGCAGTC |
| | AGCAGATCCATGTGGGCATTCTGAGCCCCACCGTGGATGATGATGACAACCGATGCCT |
| | GGTGGACGTCAACAGCCGGCCCCGGCTCATCGAATGCAGCTACGCCAAAGCCAAGAGG |
| | ATGAAGCTGCACTGGCAGTTCTCTCAGGGAGGACCCATCCAGAACCGCAAGTCCAAGC |
| | GCTGTCTGAAGCTGCAGGAGAATAGCGACCTGGAGTTCGGCTTCCAGCTGGTGTTGCA |
| | GAAGTGCTCGGGCCAGCAAGGGAGCATCACCAACGTCCTGAGGAGCCTCGCGTCCTGA |
| | CCCACCGGGGCCACTTCCGTGCTGCCTCTTTGCTACTGTGTAGCACCTGCTGCAACAT |
| | TGCCTGCTGTCCACGTGGGGTTGTTTGGAGTCTGGGGAACCAGGTTAGTGGGCCCCCA |
| | AGAAGAGCTTTTTATTTCCTATTCAATTTTCATGGAGTTTATAGAAAGATGCTGATTG |
| | GTAGGTGATGGTATGATATCAAACTATTTTGCAGTTGTAAATAG |

| | |
|---|---|
| ORF Start: ATG at 381 | ORF Stop: TGA at 2202 |

| | | |
|---|---|---|
| SEQ ID NO: 48 | 607 aa | MW at 69438.7 kD |

| | |
|---|---|
| NOV16a, | MVCTRKTKTLVSTCVILSGMTNIICLLYVGWVTNYIASVYVRGQEPAPDKKLEFDKGD |
| CG124691- | |
| 01 Protein | TLKIIERLDHLENVIKQHIQEAPAKPEEAEAEPFTDSSLFAHWGQELSPEGRRVALKQ |
| Sequence | |
| | FQYYGYNAYLSDRLPLDRPLPDLRPSGCRNLSFPDSLPEVSIVFIFVNEALSVLLRSI |
| | HSAMERTPPHLLKEIILVDDNSSNEELKEKLTEYVDKVNSQKPGFIKVVRHSKQEGLI |
| | RSRVSGWRAATAPVVALFDAHVEFNVGWAEPVLTRIKENRKRIISPSFDNIKYDNFEI |
| | EEYPLAAQGFDWELWCRYLNPPKAWWKLENSTAPIRSPALIGCFIVDRQYFQEIGLLD |
| | EGMEVYGGENVELGIRVWQCGGSVEVLPCSRIAHIERAHKPYTEDLTAHVRRNALRVA |
| | EVWMDEFKSHVYMAWNIPQEDSGIDMGDITARKALRKQLQCKTFRWYLVSVYPEMRMY |
| | SDIIAYGVLQNSLKTDLCLDQGPDTENVPIMYICHGMTPQNVYYTSSQQIHVGILSPT |
| | VDDDDNRCLVDVNSRPRLIECSYAKAKRMKLHWQFSQGGPIQNRKSKRCLKLQENSDL |
| | EFGFQLVLQKCSGQQGSITNVLRSLAS |

| | |
|---|---|
| SEQ ID NO: 49 | 2422 bp |

| | |
|---|---|
| NOV16b, | CGCCAAGGCAGCCGGCGCTGGCGATGGGAAGCGGCGTGGCCGCCGACACAGGCAGTGG |
| CG124691- | |
| 01 DNA | CAAAGTTTCCCAGACGTACACATCTGGACGCGCGGCTGCCGGCTACCCGTGACCCCTC |
| Sequence | |
| | TAGGAAGGGTTCAGGGATTTTTAATTTGGAAAAAAATCCACCTGGTTTCCTTTGTCAA |
| | GGTCTCTCCGGGTGGCCAGCGGCAGGAGCTGCAAACTTGGGCACGGCGGCTACACCGG |
| | CAGCGGACCGGGCTTTGGAGAACCTCGGGACTCAGGTGCTGAGGTGCCCAGCGGCTCC |
| | GGACGTGCTACGGGGTGCGAGCGCGGGGGAGTTCGGGGCGCACGACAAGGAAGGGCCC |
| | CCGGGAGCTCTATATGGAGGAAGGAGCCCAGAATGGTGTGCACCAGGAAGACCAAAAC |
| | TTTGGTGTCCACTTGCGTGATCCTGAGCGGCATGACTAACATCATCTGCCTGCTCTAC |
| | GTGGGCTGGGTCACCAACTACATCGCCAGCGTGTATGTGCGGGGGCAGGAGCCGGCGC |
| | CCGACAAGAAGCTGGAGGAAGACAAAGGGGACACTCTGAAGATTATTGAGCGGCTGGA |
| | CCACCTGGAGAATGTCATCAAGCAGCACATTCAAGAGGCTCCTGCCAAGCCTGAGGAG |
| | GCAGAGGCCGAGCCCTTCACAGACTCCTCTCTGTTTGCACACTGGGGCCAGGAGCTCA |
| | GCCCCGAAGGCCGGCGCGTGGCCCTGAAGCAATTCCAGTACTACGGCTACAACGCCTA |
| | CCTCAGCGACCGCCTGCCCCTGGACCGGCCCCTGCCTGACCTCAGACCCAGTGGGTGC |
| | CGTAACCTCTCATTTCCTGACAGCCTGCCAGAGGTGAGCATCGTGTTCATCTTCGTCA |
| | ATGAAGCGCTTTCAGTGCTGCTGCGCTCCATCCACTCGGCCATGGAACGCACGCCCCC |
| | ACATCTGCTCAAGGAGATCATTCTGGTGGATGACAACAGCAGTAACGAGGAACTGAAG |
| | GAGAAGCTGACCGAATATGTGGACAAGGTGAACAGCCAGAAGCCAGGCTTCATCAAAG |
| | TCGTGCGTCACAGCAAGCAGGAAGGCCTCATCCGCTCCAGGGTCAGTGGCTGGAGGGC |
| | GGCCACTGCCCCTGTGGTGGCACTCTTTGATGCCCACGTGGAGTTCAATGTGGGCTGG |
| | GCTGAACCTGTACTCACCCGCATCAAGGAGAACCGGAAGCGGATCATCTCGCCATCCT |
| | TTGATAACATCAAATATGACAACTTTGAGATAGAAGAGTACCCGCTGGCTGCCCAGGG |
| | CTTTGACTGGGAGCTGTGGTGCCGCTACCTAAATCCCCCCAAGGCCTGGTGGAAGCTG |
| | GAGAACTCCACAGCGCCAATCAGGAGCCCTGCCCTCATTGGCTGCTTCATTGTGGACC |
| | GGCAGTACTTCCAGGAGATCGGCCTGCTGGACGAAGGCATGGAAGTCTACGGGGGCGA |
| | GAATGTGGAGCTTGGGATCAGGGTGTGGCAGTGTGGCGGGAGTGTGGAGGTCCTGCCC |
| | TGCTCACGGATTGCCCACATTGAGCGAGCCCACAAGCCCTACACAGAGGACCTCACCG |
| | CCCATGTCCGCAGGAACGCTCTCAGGGTGGCTGAAGTCTGGATGGATGAATTTAAAAG |
| | CCACGTCTACATGGCATGGAACATACCGCAGGAGGACTCAGGAATTGACATGGGGGAC |
| | ATCACGGCAAGGAAGGCTCTCAGGAAACAGCTGCAGTGCAAGACCTTCCGGTGGTACC |
| | TGGTCAGCGTGTACCCAGAGATGAGGATGTACTCCGACATCATTGCCTATGGAGTGCT |
| | GCAGAATTCTCTGAAGACTGATTTGTGTCTTGACCAGGGGCCAGATACAGAGAATGTC |
| | CCCATCATGTACATCTGCCATGGGATGACGCCTCAGAACGTGTACTACACGAGCAGTC |
| | AGCAGATCCATGTGGGCATTCTGAGCCCCACCGTGGATGATGATGACAACCGATGCCT |
| | GGTGGACGTCAACAGCCGGCCCCGGCTCATCGAATGCAGCTACGCCAAAGCCAAGAGG |
| | ATGAAGCTGCACTGGCAGTTCTCTCAGGGAGGACCCATCCAGAACCGCAAGTCCAAGC |
| | GCTGTCTGAAGCTGCAGGAGAATAGCGACCTGGAGTTCGGCTTCCAGCTGGTGTTGCA |
| | GAAGTGCTCGGGCCAGCAAGGGAGCATCACCAACGTCCTGAGGAGCCTCGCGTCCTGA |
| | CCCACCGGGGCCACTTCCGTGCTGCCTCTTTGCTACTGTGTAGCACCTGCTGCAACAT |
| | TGCCTGCTGTCCACGTGGGGTTGTTTGGAGTCTGGGGAACCAGGTTAGTGGGCCCCCA |
| | AGAAGAGCTTTTTATTTCCTATTCAATTTTCATGGAGTTTATAGAAAGATGCTGATTG |
| | GTAGGTGATGGTATGATATCAAACTATTTTGCAGTTGTAAATAG |

| | |
|---|---|
| ORF Start: ATG at 381 | ORF Stop: TGA at 2202 |

| | | |
|---|---|---|
| SEQ ID NO: 50 | 607 aa | MW at 69438.7 kD |

| | |
|---|---|
| NOV16b, | MVCTRKTKTLVSTCVILSGMTNIICLLYVGWVTNYIASVYVRGQEPARDKKLEEDKGD |
| CG124691- | |
| 01 Protein | TLKIIERLDHLENVIKQHIQEAPAKPEFAEAEPFTDSSLFAHWGQELSPEGRRVALKQ |
| Sequence | |
| | FQYYGYNAYLSDRLPLDRPLPDLRPSGCRNLSFPDSLPEVSIVFIFVNEALSVLLRSI |
| | HSAMERTPPHLLKEIILVDDNSSNEELKEKLTEYVDKVNSQKPGFIKVVRHSKQEGLI |
| | RSRVSGWRAATAPVVALFDAHVEFNVGWAEPVLTRIKENRKRIISPSFDNIKYDNFEI |
| | EEYPLAAQGFDWELWCRYLNPPKAWWKLENSTAPIRSPALIGCFIVDRQYFQEIGLLD |
| | EGMEVYGGENVELGTRVWQCGGSVEVLPCSRIAHIERAHKPYTEDLTAHVRRNALRVA |
| | EVWMDEFKSHVYMAWNIPQEDSGIDMGDITARKALRKQLQCKTFRWYLVSVYPEMRMY |
| | SDIIAYGVLQNSLKTDLCLDQGPDTENVPIMYICHGMTPQNVYYTSSQQIHVGILSPT |
| | VDDDDNRCLVDVNSRPRLIECSYAKAKRMKLHWQFSQGGPIQNRKSKRCLKLQENSDL |
| | EFGFQLVLQKCSGQQGSITNVLRSLAS |

| | |
|---|---|
| SEQ ID NO: 51 | 2422 bp |

| | |
|---|---|
| NOV16c, | CGCCAAGGCAGCCGGCGCTGGCGATGGGAAGCGGCGTGGCCGCCGACACAGGCAGTGG |
| CG124691- | |
| 01 DNA | CAAAGTTTCCCAGACGTACACATCTGGACGCGCGGCTGCCGGCTACCCGTGACCCCTC |
| Sequence | |
| | TAGGAAGGGTTCAGGGATTTTTAATTTGGAAAAAAATCCACCTGGTTTCCTTTGTCAA |
| | GGTCTCTCCGGGTGGCCAGCGGCAGGAGCTGCAAACTTGGGCACGGCGGCTACACCGG |
| | CAGCGGACCGGGCTTTGGAGAACCTCGGGACTCAGGTGCTGAGGTGCCCAGCGGCTCC |
| | GGACGTGCTACGGGGTGCGAGCGCGGGGGAGTTCGGGGCGCACGACAAGGAAGGGCCC |
| | CCGGGAGCTCTATATGGAGGAAGGAGCCCAGAATGGTGTGCACCAGGAAGACCAAAAC |
| | TTTGGTGTCCACTTGCGTGATCCTGAGCGGCATGACTAACATCATCTGCCTGCTCTAC |
| | GTGGGCTGGGTCACCAACTACATCGCCAGCGTGTATGTGCGGGGGCAGGAGCCGGCGC |
| | CCGACAAGAAGCTGGAGGAAGACAAAGGGGACACTCTGAAGATTATTGAGCGGCTGGA |
| | CCACCTGGAGAATGTCATCAAGCAGCACATTCAAGAGGCTCCTGCCAAGCCTGAGGAG |
| | GCAGAGGCCGAGCCCTTCACAGACTCCTCTCTGTTTGCACACTGGGGCCAGGAGCTCA |
| | GCCCCGAAGGCCGGCGCGTGGCCCTGAAGCAATTCCAGTACTACGGCTACAACGCCTA |
| | CCTCAGCGACCGCCTGCCCCTGGACCGGCCCCTGCCTGACCTCAGACCCAGTGGGTGC |
| | CGTAACCTCTCATTTCCTGACAGCCTGCCAGAGGTGAGCATCGTGTTCATCTTCGTCA |
| | ATGAAGCGCTTTCAGTGCTGCTGCGCTCCATCCACTCGGCCATGGAACGCACGCCCCC |
| | ACATCTGCTCAAGGAGATCATTCTGGTGGATGACAACAGCAGTAACGAGGAACTGAAG |
| | GAGAAGCTGACCGAATATGTGGACAAGGTGAACAGCCAGAAGCCAGGCTTCATCAAAG |
| | TCGTGCGTCACAGCAAGCAGGAAGGCCTCATCCGCTCCAGGGTCAGTGGCTGGAGGGC |
| | GGCCACTGCCCCTGTGGTGGCACTCTTTGATGCCCACGTGGAGTTCAATGTGGGCTGG |
| | GCTGAACCTGTACTCACCCGCATCAAGGAGAACCGGAAGCGGATCATCTCGCCATCCT |
| | TTGATAACATCAAATATGACAACTTTGAGATAGAAGAGTACCCGCTGGCTGCCCAGGG |
| | CTTTGACTGGGAGCTGTGGTGCCGCTACCTAAATCCCCCCAAGGCCTGGTGGAAGCTG |
| | GAGAACTCCACAGCGCCAATCAGGAGCCCTGCCCTCATTGGCTGCTTCATTGTGGACC |
| | GGCAGTACTTCCAGGAGATCGGCCTGCTGGACGAAGGCATGGAAGTCTACGGGGGCGA |
| | GAATGTGGAGCTTGGGATCAGGGTGTGGCAGTGTGGCGGGAGTGTGGAGGTCCTGCCC |
| | TGCTCACGGATTGCCCACATTGAGCGAGCCCACAAGCCCTACACAGAGGACCTCACCG |
| | CCCATGTCCGCAGGAACGCTCTCAGGGTGGCTGAAGTCTGGATGGATGAATTTAAAAG |
| | CCACGTCTACATGGCATGGAACATACCGCAGGAGGACTCAGGAATTGACATGGGGGAC |
| | ATCACGGCAAGGAAGGCTCTCAGGAAACAGCTGCAGTGCAAGACCTTCCGGTGGTACC |
| | TGGTCAGCGTGTACCCAGAGATGAGGATGTACTCCGACATCATTGCCTATGGAGTGCT |
| | GCAGAATTCTCTGAAGACTGATTTGTGTCTTGACCAGGGGCCAGATACAGAGAATGTC |
| | CCCATCATGTACATCTGCCATGGGATGACGCCTCAGAACGTGTACTACACGAGCAGTC |
| | AGCAGATCCATGTGGGCATTCTGAGCCCCACCGTGGATGATGATGACAACCGATGCCT |
| | GGTGGACGTCAACAGCCGGCCCCGGCTCATCGAATGCAGCTACGCCAAAGCCAAGAGG |
| | ATGAAGCTGCACTGGCAGTTCTCTCAGGGAGGACCCATCCAGAACCGCAAGTCCAAGC |
| | GCTGTCTGAAGCTGCAGGAGAATAGCGACCTGGAGTTCGGCTTCCAGCTGGTGTTGCA |
| | GAAGTGCTCGGGCCAGCAAGGGAGCATCACCAACGTCCTGAGGAGCCTCGCGTCCTGA |
| | CCCACCGGGGCCACTTCCGTGCTGCCTCTTTGCTACTGTGTAGCACCTGCTGCAACAT |
| | TGCCTGCTGTCCACGTGGGGTTGTTTGGAGTCTGGGGAACCAGGTTAGTGGGCCCCCA |
| | AGAAGAGCTTTTTATTTCCTATTCAATTTTCATGGAGTTTATAGAAAGATGCTGATTG |
| | GTAGGTGATGGTATGATATCAAACTATTTTGCAGTTGTAAATAG |

| | |
|---|---|
| ORF Start: ATG at 381 | ORF Stop: TGA at 2202 |

| | | |
|---|---|---|
| SEQ ID NO: 52 | 607 aa | MW at 69438.7 kD |

| | |
|---|---|
| NOV16c, | MVCTRKTKTLVSTCVILSGMTNIICLLYVGWVTNYIASVYVRGQEPARDKKLEEDKGD |
| CG 124691- | |
| 01 Protein | TLKIIERLDHLENVIKQHIQEARAKPEEAEAEPFTDSSLFAHWGQELSPEGRRVALKQ |
| Sequence | |
| | FQYYGYNAYLSDRLPLDRPLPDLRPSGCRNLSFPDSLPEVSIVFIFVNEALSVLLRSI |
| | HSAMERTPPHLLKEIILVDDNSSNEELKEKLTEYVDKVNSQKPGFIKVVRHSKQEGLI |
| | RSRVSGWRAATAPVVALFDAHVEFNVGWAEPVLTRIKENRKRIISPSFDNIKYDNFEI |
| | EEYPLAAQGFDWELWCRYLNPPKAWWKLENSTAPIRSPALIGCFIVDRQYFQEIGLLD |
| | EGMEVYGGENVELGIRVWQCGGSVEVLPCSRIAHIERAHKPYTEDLTAHVRRNALRVA |
| | EVWMDEFKSHVYMAWNIPQEDSGIDMGDITARKALRKQLQCKTFRWYLVSVYPEMRMY |
| | SDIIAYGVLQNSLKTDLCLDQGPDTENVPIMYTCHGMTPQNVYYTSSQQIHVGILSPT |
| | VDDDDNRCLVDVNSRPRLIECSYAKAKRMKLHWQFSQGGPIQNRKSKRCLKLQENSDL |
| | EFGFQLVLQKCSGQQGSITNVLRSLAS |

Sequence comparison of the above protean sequence yields the following sequence relationships shown in Table 16D.

TABLE 16B
Comparison of NOV16a against NOV16b and NOV16c.

| Protein | NOV16a Residues/ | Identities/Similarities |
|---|---|---|
| Sequence | Match Residues | for the Matched Region |
| NOV16b | 1 . . . 607 | 580/607 (95%) |
| | 1 . . . 607 | 580/607 (95%) |
| NOV16c | 1 . . . 607 | 580/607 (95%) |
| | 1 . . . 607 | 580/607 (95%) |

Further analysis of the NOV16a protein yielded the following properties shown in Table 16C.

TABLE 16C
Protein Sequence Properties NOV16a

| | |
|---|---|
| PSort | 0.6850 probability located in endoplasmic reticulum |
| analysis: | (membrane); 0.6400 probability located in plasma membrane; |
| | 0.4600 probability located in Golgi body; 0.1000 |
| | probability located in endoplasmic reticulum (lumen) |
| SignalP | Cleavage site between residues 44 and 45 |
| analysis: | |

A search of the NOV16a protein against the Geneseq database a proprietary database that contains sequences published in patents and patent publication yielded several homologous proteins shown in Table 16D.

TABLE 16D
Geneseq Results for NOV16a

| | | NOV16a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAM41675 | Human poly- | 105 . . . 603 | 283/503 (56%) | e−169 |
| | peptide SEQ ID | 65 . . . 560 | 368/503 (72%) | |
| | NO 6606—Homo | | | |
| | sapiens, 560 aa. | | | |
| | [WO200153312- | | | |
| | A1, 26 JUL. | | | |
| | 2001] | | | |
| ABB04283 | Human N-acetyl- | 351 . . . 607 | 252/257 (98%) | e−149 |
| | galactosamine | 1 . . . 257 | 254/257 (98%) | |
| | transferase-28 | | | |
| | polypeptide— | | | |
| | Homo sapiens, | | | |
| | 257 aa. | | | |
| | [WO200190369- | | | |
| | A1, 29 NOV. | | | |
| | 2001] | | | |
| AAM40398 | Human poly- | 236 . . . 603 | 201/371 (54%) | e−124 |
| | peptide SEQ ID | 29 . . . 398 | 274/371 (73%) | |
| | NO 3543—Homo | | | |
| | sapiens, 402 aa. | | | |
| | [WO200153312- | | | |
| | A1, 26 JUL. | | | |
| | 2001] | | | |
| AAB40597 | Human ORFX | 360 . . . 553 | 187/195 (95%) | e−106 |
| | ORF361 poly- | 1 . . . 193 | 190/195 (96%) | |
| | peptide sequence | | | |
| | SEQ ID NO: | | | |
| | 722—Homo | | | |
| | sapiens, 193 aa. | | | |
| | [WO200058473- | | | |
| | A2, 5 OCT. | | | |
| | 2000] | | | |
| AAB41739 | Human ORFX | 174 . . . 441 | 171/269 (63%) | e−105 |
| | ORF1503 poly- | 1 . . . 266 | 216/269 (79%) | |
| | peptide sequence | | | |
| | SEQ ID NO: | | | |
| | 3006—Homo | | | |
| | sapiens, 266 aa. | | | |
| | [WO200058473- | | | |
| | A2, 5 OCT. | | | |
| | 2000] | | | |

In a BLAST search of public sequence databases, the NOV 16a protein was found to have homology to the proteins shown in the BLASTP data in Table 16E.

TABLE 16E
Public BLASTP Results for NOV16a

| | | NOV16a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q9HCQ5 | UDP-GalNAc: | 57 . . . 603 | 292/552 (52%) | e−169 |
| | polypeptide N- | 58 . . . 599 | 390/552 (69%) | |
| | acetylgalactos- | | | |
| | aminyl- | | | |
| | transferase— | | | |
| | Homo sapiens | | | |
| | (Human), 603 aa. | | | |
| AAM62306 | Putative poly- | 105 . . . 603 | 283/503 (56%) | e−169 |
| | peptide N-acetyl- | 103 . . . 598 | 368/503 (72%) | |
| | galactosaminyl- | | | |
| | transferase— | | | |
| | Homo sapiens | | | |
| | (Human), 598 aa. | | | |
| Q9GM01 | UDP-GalNAc: | 70 . . . 603 | 287/540 (53%) | e−168 |
| | polypeptide N- | 72 . . . 602 | 385/540 (71%) | |
| | acetylgalactos- | | | |
| | aminyl- | | | |
| | transferase— | | | |
| | Macaca | | | |
| | fascicularis | | | |
| | (Crab eating | | | |
| | macaque) | | | |
| | (Cynomolgus | | | |
| | monkey), 606 aa. | | | |
| AAM62404 | Williams-Beuren | 105 . . . 603 | 285/503 (56%) | e−168 |
| | syndrome critical | 103 . . . 596 | 368/503 (72%) | |
| | region gene 17— | | | |
| | Mus musculus | | | |
| | (Mouse), 596 aa. | | | |
| Q9NY28 | UDP-N-acetyl- | 44 . . . 603 | 274/562 (48%) | e−159 |
| | alpha-D-galactos- | 80 . . . 629 | 380/562 (66%) | |
| | amine:poly- | | | |
| | peptide N-acetyl | | | |
| | galactosaminyl- | | | |
| | transferase 8— | | | |
| | Homo sapiens | | | |
| | (Human), 637 aa. | | | |

PFam analysis predicts that the NOV 16a protein contains tie domains shown in tie Table 16F

TABLE 16F
Domain Analysis of NOV16a

| Pfam | NOV16a | Identities/Similarities | Expect |
|---|---|---|---|
| Domain | Match Region | for the Matched Region | Value |
| Glycos_transf_2 | 157 . . . 345 | 42/192 (22%) | 1.4e−24 |
| | | 136/192 (71%) | |

### Example 17

The NOV17 clone was analyzed, and their nucleotide and encoded polypeptide sequences are shown in Table 17A.

TABLE 17A
NOV17 Sequence Analysis

| | |
|---|---|
| SEQ ID NO: 53 | 1132 bp |

| | |
|---|---|
| NOV17a, | GTTATGAAGTGCAAGGCTGCAGTTGCTTGGGAGGCTGGAAAGCCTCTCTCCATAGAGG |
| CG125169- | |
| 01 DNA | AGATAGAGGTGGCACCCCCAAAGGCTCATGAAGTTCGAATCAAGATCATTGCCACTGC |
| Sequence | |
| | GGTTTGCCACACCGACGCCTATACCCTGAGGGGAGCTGATCCTGAGGGTTGTTTTCCA |
| | GTGATCTTGGGACATGAAGGTGCCGGAATTGAGGAAAGTGTTGGCGAGGGAGTTACTA |
| | AGCTGAAGGCGGGTGACACTGTCATCCCACTTTACATCCCACAGTGTGGAGAATGCAA |
| | ATTTTGTCTATATCCTAAAACTAACCTTTGCCAGAAGATAAGAGTCACTCAAGGGAAA |
| | GGATTAATGCCAGATGGTACCAGCAGATTTACTTGCAAAGGAAAGACAATTTTGCGTT |
| | ACATGGGAACCAGCACATTTTCTGAATACACAGTTGTAGCTGATATCTCTGTTGCTAA |
| | AATAGATCCTTTAGCACCTTTGGATAAACTCTGCCTTCTAGGTTGTGGCATTTCAGCT |
| | GGTGATGGTGCTGCTGTGAACACTGCCAAGGTGGAACCTGGCTCTGTTTGTGCCGTCT |
| | TTGGTCTGGGAGGAGTTGGATTGGCAGTTATCAAGGGCTGTAAAGTGGCTGGTGCATC |
| | CCGGATCATTGGTGTGGACATCAATAAAGATAAATTTGCAAGGGCCAAAGAGTTTGGA |
| | GCCACTGAATGTATTAACCCTCAGGGTTTTAGTAAACCCATCCAGGAAGGGCTCATTG |
| | AGACGACTGATGGAGGAGTGGACTATTCCTTTGAATGTATTGGTAATGTGAAGGTCAT |
| | GAGAGCAGCACTTGAGGCTTATCACAAGGGCTGGGGAGTCAGCGTGGTGGTTGGAGTA |
| | GCTGCTTCAGGTGAAGAAATTGCCACTCGTCCATTCCAGCTGGTAACAGGTCGCACAT |
| | GGAAAGGAACTGCCTTTGGAGGATGGAAGAGTGTAGAAAGTGTCCCAAAGTTGGTGTC |
| | TGAATATATGTCCAAAAAAATAAAAGTTGATGAATTTGTGACTCACAATCTGTCTTTT |
| | GGTGAAATTAACAAAGCCTTTCAACTGATGCATTCTGGAAAGAGCATTCGAACTGTTG |
| | TAAAGATTTAATTCAAAAGAGAAAAATAAT |

| | |
|---|---|
| ORF Start: ATG at 4 | ORF Stop: TAA at 1111 |

| | | |
|---|---|---|
| SEQ ID NO: 54 | 369 aa | MW at 39154.1 kD |

| | |
|---|---|
| NOV17a, | MKCKAAVAWEAGKRLSIEEIEVAPPKAHEVRIKIIATAVCHTDAYTLRGADPEGCFPV |
| CG125169- | |
| 01 Protein | ILGHEGAGIEESVGEGVTKLKAGDTVIPLYIPQCGECKFCLYPKTNLCQKIRVTQGKG |
| Sequence | |
| | LMPDGTSRFTCKGKTILRYMGTSTFSEYTVVADISVAKIDPLAPLDKLCLLGCGISAG |
| | DGAAVNTAKVEPGSVCAVFGLGGVGLAVIKGCKVAGASRIIGVDINKDKFARAKEFGA |
| | TECINPQGFSKPIQEGLIETTDGGVDYSFECIGNVKVMRAALEAYHKGWGVSVVVGVA |
| | ASGEEIATRPFQLVTGRTWKGTAFGGWKSVESVPKLVSEYMSKKIKVDEFVTHNLSFG |
| | EINKAFQLMHSGKSIRTVVKI |

Further analysis of the NOV17a protein yielded the following properties shown in Table 17B.

TABLE 17B
Protein Sequence Properties NOV17a

| | |
|---|---|
| PSort | 0.7000 probability located in plasma membrane; 0.2000 |
| analysis: | probability located in endoplasmic reticulum (membrane); |
| | 0.1000 probability located in mitochondrial inner membrane; |
| | 0.0692 probability located in microbody (peroxisome) |
| SignalP | No Known Signal Sequence Predicted |
| analysis: | |

A search of the NOV17a protein against the Geneseq database, a proprietary database that contains sequences published in patents and patent publication, yielded several homologous proteins shown in Table 17C.

TABLE 17C
Geneseq Results for NOV17a

| | | NOV17a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAB43405 | Human cancer | 1 . . . 369 | 351/369 (95%) | 0.0 |
| | associated protein | 15 . . . 383 | 356/369 (96%) | |
| | sequence | | | |
| | SEQ ID NO: | | | |
| | 850—Homo | | | |
| | sapiens, 383 aa. | | | |
| | [WO200055350- | | | |
| | A1, 21 SEP. | | | |
| | 2000] | | | |
| ABB62511 | Drosophila | 3 . . . 368 | 258/368 (70%) | e−153 |
| | melanogaster | 11 . . . 378 | 301/368 (81%) | |
| | polypeptide SEQ | | | |
| | ID NO 14325— | | | |
| | Drosophila | | | |
| | melanogaster, | | | |
| | 379 aa. | | | |
| | [WO200171042- | | | |
| | A2, 27 SEP. | | | |
| | 2001] | | | |
| AAG45942 | Arabidopsis | 3 . . . 367 | 248/366 (67%) | e−144 |
| | thaliana protein | 10 . . . 375 | 291/366 (78%) | |
| | fragment SEQ ID | | | |
| | NO: 57741— | | | |
| | Arabidopsis | | | |
| | thaliana, 379 aa. | | | |
| | [EP1033405-A2, | | | |
| | 6 SEP. 2000] | | | |
| AAG45941 | Arabidopsis | 3 . . . 367 | 248/366 (67%) | e−144 |
| | thaliana protein | 26 . . . 391 | 291/366 (78%) | |
| | fragment SEQ ID | | | |
| | NO: 57740— | | | |
| | Arabidopsis | | | |
| | thaliana, 395 aa. | | | |
| | [EP1033405-A2, | | | |
| | 6 SEP. 2000] | | | |
| AAG16746 | Arabidopsis | 3 . . . 367 | 248/366 (67%) | e−144 |
| | thaliana protein | 10 . . . 375 | 291/366 (78%) | |
| | fragment SEQ ID | | | |
| | NO: 17509— | | | |
| | Arabidopsis | | | |
| | thaliana, 379 aa. | | | |
| | [EP1033405-A2, | | | |
| | 6 SEP. 2000] | | | |

In a BLAST search of public sequence databases, the NOV17a protein was found to have homology to the proteins shown in the BLAST data in Table 17D.

TABLE 17D
Public BLASTP Results for NOV17a

| | | NOV17a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| CAC27318 | Sequence 53 | 1 . . . 369 | 353/369 (95%) | 0.0 |
| | from Patent | 6 . . . 374 | 357/369 (96%) | |
| | WO0102600— | | | |
| | Homo sapiens | | | |
| | (Human), 374 aa. | | | |
| P11766 | Alcohol | 1 . . . 369 | 353/369 (95%) | 0.0 |
| | dehydrogenase class | 5 . . . 373 | 357/369 (96%) | |
| | III chi chain | | | |
| | (EC 1.1.1.1) | | | |
| | (Glutathione- | | | |
| | dependent | | | |
| | formaldehyde | | | |
| | dehydrogenase) | | | |
| | (EC 1.2.1.1) | | | |
| | (FDH)—Homo | | | |
| | sapiens (Human), | | | |
| | 373 aa. | | | |
| P19854 | Alcohol | 1 . . . 369 | 338/369 (91%) | 0.0 |
| | dehydrogenase | 5 . . . 373 | 354/369 (95%) | |
| | class III chain | | | |
| | (EC 1.1.1.1) | | | |
| | (Glutathione- | | | |
| | dependent | | | |
| | formaldehyde | | | |
| | dehydrogenase) | | | |
| | (EC 1.2.1.1) (FDH) | | | |
| | (FALDH)—Equus | | | |
| | caballus (Horse), | | | |
| | 373 aa. | | | |
| O19053 | Alcohol | 1 . . . 369 | 337/369 (91%) | 0.0 |
| | dehydrogenase | 5 . . . 373 | 347/369 (93%) | |
| | class III chain | | | |
| | (EC 1.1.1.1) | | | |
| | (Glutathione- | | | |
| | dependent | | | |
| | formaldehyde | | | |
| | dehydrogenase) | | | |
| | (EC 1.2.1.1) (FDH) | | | |
| | (FALDH)— | | | |
| | Oryctolagus | | | |
| | cuniculus | | | |
| | (Rabbit), 373 aa. | | | |
| P12711 | Alcohol | 1 . . .369 | 337/369 (91%) | 0.0 |
| | dehydrogenase | 5 . . . 373 | 350/369 (94%) | |
| | class III (EC | | | |
| | 1.1.1.1) (Alcohol | | | |
| | dehydrogenase 2) | | | |
| | (Glutathione- | | | |
| | dependent | | | |
| | formaldehyde | | | |
| | dehydrogenase) | | | |
| | (EC 1.2.1.1) (FDH) | | | |
| | (FALDH) (Alcohol | | | |
| | dehydrogenase- | | | |
| | B2)—Rattus | | | |
| | norvegicus (Rat), | | | |
| | 373 aa. | | | |

PFam analysis predicts that the NOV17a protein contains the domains shown in the Table 17E.

TABLE 17E
Domain Analysis of NOV17a

| Pfam | NOV17a | Identities/Similarities | Expect |
|---|---|---|---|
| Domain | Match Region | for the Matched Region | Value |
| adh_zinc | 14 . . . 369 | 146/463 (32%) | 1.9e−138 |
| | | 324/463 (70%) | |

### Example 18

The NOV18 clone was analyzed, and the neucleoticle and encoded polypeptide sequences are shown in Table 18A.

TABLE 18A
NOV18 Sequence Analysis

| | |
|---|---|
| SEQ ID NO: 55 | 701 bp |

| | |
|---|---|
| NOV18a, | GCGGTGTATGTGCGGCAATAACATGTCAACCCCGCTGCCCACCATCGTGCCCGCCCCC |
| CG125197- | |
| 01 DNA | CGGAAGGCCACCACTGAGGTGATTTTCCTGCATGGATTGGGAGATACTGGGCACGGAT |
| Sequence | |
| | GGGCAGAAGCCTTTGCCGGTATCATAAGTTCACATATCAAATATATCTGCCCGCATGC |
| | GCCTGTTAGGCCTGTTACATTAAATATGAACATAGCTATGCCTTCATGGTTTGATATT |
| | ATTGGGCTTTCACCAGATTCACAGGAGGATGAATCTGGGATTAAACAGGCAGCACAAA |
| | ATATAAAAGCTTTGATTGATCAAGAAGTGAAGAATGGCATTCCTTCTAACAGAATTAT |
| | TTTGGGAGGGTTTTCTCAGGGAGGAGCTTTATCTTTATATACTGCCCTTACCACGCAC |
| | CAGAAACTGGCAGGTGTCACTGCACTCAATTGCTGGCTTCCACTTTGGGCTTCCTTTC |
| | CACAGGGTCCTATCGGTGGTGCTAATAGAGATATTTCTATTCTCCAGTGCCACGGGGA |
| | TTGTGACCCTTTGGTTCCCCTGATGTTTGGTTCTCTTACGGTTGAAAAACTAAAAACA |
| | TTGGTGAATCCAGCCAATGTGACCTTTAAAACCTATGAAGGTATGATGCACAGTTCGT |
| | GTCAACAGGAAATGATGAATGTCAAGCAATTCATTGATAAACTCCTACCTCCAATTGA |
| | TTGAC |

| | |
|---|---|
| ORF Start: ATG at 8 | ORF Stop: TGA at 698 |

| | | |
|---|---|---|
| SEQ ID NO: 56 | 230 aa | MW at 24848.5 kD |

| | |
|---|---|
| NOV18a, | MCGNNMSTPLPTIVPAPRKATTEVIFLHGLGDTGHGWAEAFAGIISSHIKYICPHAPV |
| CG125197- | |
| 01 Protein | RPVTLNMNIAMPSWFDIIGLSPDSQEDESGIKQAAQNIKALIDQEVKNGIPSNRIILG |
| Sequence | |
| | GFSQGGALSLYTALTTHQKLAGVTALNCWLPLWASFPQGPIGGANRDISILQCHGDCD |
| | PLVPLMFGSLTVEKLKTLVNPANVTFKTYEGMMHSSCQQEMMNVKQFIDKLLPPID |

Further analysis of the NOV18a protein yielded the following properties shown in Table 18IB.

TABLE 18B
Protein Sequence Properties NOV18a

| | |
|---|---|
| PSort | 0.6500 probability located in cytoplasm; 0.2605 probability |
| analysis: | located in lysosome (lumen); 0.1000 probability located in |
| | mitochondrial matrix space; 0.0000 probability located in |
| | endoplasmic reticulum (membrane) |
| SignalP | No Known Signal Sequence Predicted |
| analysis: | |

A search of the NOV18a protein against the Geneseq database, a proprietary database that contains sequences published in patents and patent publication, yielded several homologous proteins shown in Table 18C.

TABLE 18C
Geneseq Results for NOV18a

| | | NOV18a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAU85134 | Human lysophospholipase I #2-Homo | 1 . . . 230 | 219/230 (95%) | e−127 |
| | sapiens, 230 aa. [WO200210185-A1, | 1 . . . 230 | 223/230 (96%) | |
| | 7 Feb. 2002] | | | |
| AAU85132 | Human lysophospholipase I #1-Homo | 1 . . . 230 | 219/230 (95%) | e−127 |
| | sapiens, 230 aa. [WO200210185-A1, | 1 . . . 230 | 223/230 (96%) | |
| | 7 Feb. 2002] | | | |
| ABG07277 | Novel human diagnostic protein #7268- | 1 . . . 230 | 219/230 (95%) | e−127 |
| | Homo sapiens, 275 aa. [WO200175067- | 46 . . . 275 | 223/230 (96%) | |
| | A2, 11 Oct. 2001] | | | |
| ABG07277 | Novel human diagnostic protein #7268- | 1 . . . 230 | 219/230 (95%) | e−127 |
| | Homo sapiens, 275 aa. [WO200175067- | 46 . . . 275 | 223/230 (96%) | |
| | A2, 11 Oct. 2001] | | | |
| AAB53451 | Human colon cancer antigen protein | 1 . . . 230 | 219/230 (95%) | e−127 |
| | sequence SEQ ID NO: 991-Homo | 34 . . . 263 | 223/230 (96%) | |
| | sapiens, 263 aa. [WO200055351-A1, | | | |
| | 21 Sep. 2000] | | | |

In a BLAST search of public sequence databases, the NOV 18a protein was found to have homologs,y to the proteins shown in the BLASTP data in Table 18D.

TABLE 18D
Public BLASTP Results for NOV18a

| | | NOV18a | | |
|---|---|---|---|---|
| Protein | | Residues/ | Identities/ | |
| Accession | | Match | Similarities for | Expect |
| Number | Protein/Organism/Length | Residues | the Matched Portion | Value |
| O75608 | Lysophospholipase (Acyl-protein | 1 . . . 230 | 219/230 (95%) | e−127 |
| | thioesterase-1) (Lysophospholipase I)- | 1 . . . 230 | 223/230 (96%) | |
| | Homo sapiens (Human), 230 aa. | | | |
| O77821 | Calcium-independent phospholipase | 1 . . . 230 | 202/230 (87%) | e−119 |
| | A2 isoform 2-Oryctolagus cuniculus | 1 . . . 230 | 213/230 (91%) | |
| | (Rabbit), 230 aa. | | | |
| P70470 | LYSOPHOSPHOLIPASE-Rattus | 1 . . . 230 | 203/230 (88%) | e−118 |
| | norvegicus (Rat), 230 aa. | 1 . . . 230 | 213/230 (92%) | |
| O77820 | Calcium-independent phospholipase | 14 . . . 230 | 202/217 (93%) | e−116 |
| | A2 isoform 1-Oryctolagus cuniculus | 3 . . . 219 | 207/217 (95%) | |
| | (Rabbit), 219 aa (fragment). | | | |
| Q9UQF9 | Lysophospholipase isoform-Homo | 1 . . . 230 | 204/230 (88%) | e−114 |
| | sapiens (Human), 214 aa. | 1 . . . 214 | 207/230 (89%) | |

PFam analysis predicts that the NOV18a protein contains the domains shown in the Table 18E.

TABLE 18E
Domain Analysis of NOV18a

| | | Identities/ | |
|---|---|---|---|
| Pfam | NOV18a | Similarities for | Expect |
| Domain | Match Region | the Matched Region | Value |
| abhydrolase_2 | 10 . . . 226 | 123/236 (52%) | 1.3e−108 |
| | | 193/236 (82%) | |

### Example 19

The NOV19 clone was analyzed, and the nucleotide and encoded polypeptide sequences are shown in Table 19A.

TABLE 19A

NOV19 Sequence Analysis

| | |
|---|---|
| SEQ ID NO: 57 | 2475 bp |

| | |
|---|---|
| NOV19a, | ACTCACTATAGGGCTCGAGCGGAGCTGCTGGCTGGAGAGGAGGGTGGACGAAGCTCTC |
| CG 125215- | |
| 01 DNA | TCTAGAAAGACATCCTGAGAGGACTTGGCAGGCCTGAATATGCATTGGCTGCGAAAAG |
| Sequence | |
| | TTCAGGGACTTTGCACCCTGTGGGGTACTCAGATGTCCAGCCGCACTCTCTACATTAA |
| | TAGTAGGCAACTGGTGTCCCTGCAGTGGGGCCACCAGGAAGTGCCGGCCAAGTTTAAC |
| | TTTGCTAGTGATGTGTTGGATCACTGGGCTGACATGGAGAAGGCTGGCAAGCGACTCC |
| | CAAGCCCAGCCCTGTGGTGGGTGAATGGGAAGGGGAAGGAATTAATGTGGAATTTCAG |
| | AGAACTGAGTGAAAACAGCCAGCAGGCAGCCAACGTCCTCTCGGGAGCCTGTGGCCTG |
| | CAGCGTGGGGATCGTGTGGCAGTGGTGCTGCCCCGAGTGCCTGAGTGGTGGCTGGTGA |
| | TCCTGGGCTGCATTCGAGCAGGTCTCATCTTTATGCCTGGAACCATCCAGATGAAATC |
| | CACTGACATACTGTATAGGTTGCAGATGTCTAAGGCCAAGGCTATTGTTGCTGGGGAT |
| | GAAGTCATCCAAGAAGTGGACACAGTGGCATCTGAATGTCCTTCTCTGAGAATTAAGC |
| | TACTGGTGTCTGAGAAAAGCTGTGATGGGTGGCTGAACTTCAAGAAACTACTAAATGA |
| | GGCATCCACCACTCATCACTGTGTGGAGACTGGAAGCCAGGAAGCATCTGCCATCTAC |
| | TTCACTAGTGGGACCAGTGGTCTTCCCAAGATGGCAGAACATTCCTACTCGAGCCTGG |
| | GCCTCAAGGCCAAGATGGATGCTGGTTGGACAGGCCTGCAAGCCTCTGATATAATGTG |
| | GACCATATCAGACACAGGTTGGATACTGAACATCTTGGGCTCACTTTTGGAATCTTGG |
| | ACATTAGGAGCATGCACATTTGTTCATCTCTTGCCAAAGTTTGACCCACTGGTTATTC |
| | TAAAGACACTCTCCAGTTATCCAATCAAGAGTATGATGGGTGCCCCTATTGTTTACCG |
| | GATGTTGCTACAGCAGGATCTTTCCAGTTACAAGTTCCCCCATCTACAGAACTGCCTC |
| | GCTGGAGGGGAGTCCCTTCTTCCAGAAACTCTGGAGAACTGGAGGGCCCAGACAGGAC |
| | TGGACATCCGAGAATTCTATGGCCAGACAGAAACGGGATTAACTTGCATGGTTTCCAA |
| | GACAATGAAAATCAAACCAGGATACATGGGAACGGCTGCTTCCTGTTATGATGTACAG |
| | GTTATAGATGATAAGGGCAACGTCCTGCCCCCCGGCACAGAAGGAGACATTGGCATCA |
| | GGGTCAAACCCATCAGGCCTATAGGCATCTTCTCTGGCTATGTGGAAAATCCCGACAA |
| | GACAGCAGCCAACATTCGAGGAGACTTTTGGCTCCTTGGAGACCGGGGAATCAAAGAT |
| | GAAGATGGGTATTTCCAGTTTATGGGACGGGCAGATGATATCATTAACTCCAGCGGGT |
| | ACCGGATTGGACCCTCGGAGGTAGAGAATGCACTGATGAAGCACCCTGCTGTGGTTGA |
| | GACGGCTGTGATCAGCAGCCCAGACCCCGTCCGAGGAGAGGTGGTGAAGGCATTTGTG |
| | ATACTGGCCTCGCAGTTCCTATCCCATGACCCAGAACAGCTCACCAAGGAGCTGCAGC |
| | AGCATGTGAAGTCAGTGACAGCCCCATACAAGTACCCAAGAAAGATAGAGTTTGTCTT |
| | GAACCTGCCCAAGACTGTCACAGGGAAAATTCAACGAACCAAACTTCGAGACAAGGAG |
| | TGGAAGATGTCCGGAAAAGCCCGTGCGCAGTGAGACATCTAGGAGACATTCATTTGGA |
| | TTCCCCTCTTCTTTCTCTTTCTTTTCCCTTTGGGCCCTTGGCCTTACTATGATGATAT |
| | GAGATTCTTTATGAAAGAACATGAATGTAAGTTTGTCTTGCCCTGGTTATTAGCCTTG |
| | GTTATTAGCACAAAACTTTACCATGTTAGATGTTGAAAGAAGAAAGGGAAGGAATGAG |
| | AGAGAGTGAAAAGGAGAGGGTAACAGAAAAAAAGGAAAGAAAAGTAAGTCAGGGAAAT |
| | ATTAAAAACTGCAAGGGAAAGCAATTGAAAAAGAAATAAAGTAGGGAAAGAAGGAGAG |
| | AGGAAGCAAGGGAAGGAGGAAGAAAGGAAAGAGGAGATGAAAGGGGGAGAAAAGATAG |
| | AAGAAAAATAATTGAAGGGAGAATCAGAAAAATAAAGAGAAGAAAGGAAAGAAATAAA |
| | GAGAGAAAGAGAAAGAAGAAAGAGCAAAAGAACACAAGAAAGAAAGAGAGGGAGAAAG |
| | AGAGGGAGAAAGGGAGAGAAAAAAATTGTAAAAATAAAAATAGTAAAAGAAACTGATA |
| | ACGAAAAGTAATGGAAGACAGGAAGAAAAGATAGAAGAAAAATAATTGAAGGGAGAAT |
| | CAGAAAAATAAAGAGAAGAAAGGAAAGAAATAAAGAGAG |

| | |
|---|---|
| ORF Start: ATG at 98 | ORF Stop: TGA at 1829 |

| | | |
|---|---|---|
| SEQ ID NO: 58 | 577 aa | MW at 64224.5 kD |

| | |
|---|---|
| NOV19a, | MHWLRKVQGLCTLWGTQMSSRTLYINSRQLVSLQWGHQEVPAKFNFASDVLDHWADME |
| CG125215- | |
| 01 Protein | KAGKRLPSPALWWVNGKGKELMWNFRELSENSQQAANVLSGACGLQRGDRVAVVLPRV |
| Sequence | |
| | PEWWLVILGCIRAGLIFMPGTIQMKSTDILYRLQMSKAKAIVAGDEVIQEVDTVASEC |
| | PSLRIKLLVSEKSCDGWLNFKKLLNEASTTHHCVETGSQEASAIYFTSGTSGLPKMAE |
| | HSYSSLGLKAKMDAGWTGLQASDIMWTISDTGWILNILGSLLESWTLGACTFVHLLPK |
| | FDPLVILKTLSSYPIKSMMGAPIVYRMLLQQDLSSYKFPHLQNCLAGGESLLPETLEN |
| | WRAQTGLDIREFYGQTETGLTCMVSKTMKIKPGYMGTAASCYDVQVIDDKGNVLPPGT |
| | EGDTGIRVKPIRPIGIFSGYVENPDKTAANIRGDFWLLGDRGIKDEDGYFQFMGRADD |
| | IINSSGYRIGPSEVENALMKHPAVVETAVISSPDPVRGEVVKAFVILASQFLSHDPEQ |
| | LTKELQQHVKSVTAPYKYPRKIEFVLNLPKTVTGKIQRTKLRDKEWKMSGKARAQ |

| | |
|---|---|
| SEQ ID NO: 59 | 1878 bp |

| | |
|---|---|
| NOV19b, | AGGGTGGACGAAGCTCTCTCTAGAAAGACATCCTGAGAGGACTTCGCAGGCCTGAACA |
| CG125215- | |
| 02 DNA | TGCATTGGCTGCGAAAAGTTCAGGGACTTTGCACCCTGTGGGGTACTCAGATGTCCAG |
| Sequence | |
| | CCGCACTCTCTACATTAATAGTAGGCAACTGGTGTCCCTGCAGTGGGGCCACCAGGAA |
| | GTTCCGGCCAAGTTTAACTTTGCTAGTGATGTGTTGGATCACTGGGCTGACATGGAGA |
| | AGGCTGGCAAGCGACTCCCAAGCCCAGCCCTGTGGTGGGTGAATGGGAAGGGGAAGGA |
| | ATTAATGTGGAATTTCAGAGAACTGAGTGAAAACAGCCGGCAGGCAGCCAACGTCCTC |
| | TCGGGAGCCTGTGGCCTGCAGCGTGGGGATCGTGTGGCAGTGATGCTGCCCCGAGTGC |
| | CTGAGTGGTGGCTGGTGATCCTGGGCTGCATTCGAGCAGGTCTCATCTTTATGCCTGG |
| | AACCATCCAGATGAAATCCACTGACATACTGTATAGGTTGCAGATGTCTAAGGCCAAG |
| | GCTATTGTTGCTGGGGATGAAGTCATCCAAGAAGTGGACACAGTGGCATCTGAATGTC |
| | CTTCTCTGAGAATTAAGCTACTGGTGTCTGAGAAAAGCTGCGATGGGTGGCTGAACTT |
| | CAAGAAACTACTAAATGAGGCATCCACCACTCATCACTGTGTGGAGACTGGAAGCCAG |
| | GAAGCATCTGCCATCTACTTCACTAGTGGGACCAGTGGTCTTCCCAAGATGGCAGAAC |
| | ATTCCTACTCGAGCCTGGGCCTCAAGGCCAAGATGGATGCTGGTTGGACAGGCCTGCA |
| | AGCCTCTGATATAATGTGGACCATATCAGACACAGGTTGGATACTGAACATCTTGGGC |
| | TCACTTTTGGAATCTTGGACATTAGGAGCATGCACATTTGTTCATCTCTTGCCAAAGT |
| | TTGACCCACTGGTTATTCTAAAGACACTCTCCAGTTATCCAATCAAGAGTATGATGGG |
| | TGCCCCTATTGTTTACCGGATGTTGCTACAGCAGGATCTTTCCAGTTACAAGTTCCCC |
| | CATCTACAGAACTGCCTCGCTGGAGGGGAGTCCCTTCTTCCAGAAACTCTGGAGAACT |
| | GGAGGGCCCAGACAGGACTGGACATCCGAGAATTCTATGGCCAGACAGAAACGGGATT |
| | AACTTGCATGGTTTCCAAGACAATGAAAATCAAACCAGGATACATGGGAACGGCTGCT |
| | TCCTGTTATGATGTACAGGTTATAGATGATAAGGGCAACGTCCTGCCCCCCGGCACAG |
| | AAGGAGACATTGGCATCAGGGTCAAACCCATCAGGCCTATAGGCATCTTCTCTGGCTA |
| | TGTGGAAAATCCCGACAAGACAGCAGCCAACATTCGAGGAGACTTTTGGCTCCTTGGA |
| | GACCGGGGAATCAAAGATGAAGATGGGTATTTCCAGTTTATGGGACGGGCAGATGATA |
| | TCATTAACTCCAGCGGGTACCGGATTGGACCCTCGGAGGTAGAGAATGCACTGATGAA |
| | GCACCCTGCTGTGGTTGAGACGGCTGTGATCAGCAGCCCAGACCCCGTCCGAGGAGAG |
| | GTGGTGAAGGCATTTGTGATACTGGCCTCGCAGTTCCTATCCCATGACCCAGAACAGC |
| | TCACCAAGGAGCTGCAGCAGCATGTGAAGTCAGTGACAGCCCCATACAAGTACCCAAG |
| | AAAGATAGAGTTTGTCTTGAACCTGCCCAAGACTGTCACAGGGAAAATTCAACGAACC |
| | AAACTTCGAGACAAGGAGTGGAAGATGTCCGGAAAAGCCCGTGCGCAGTGAGGCGTCT |
| | AGGAGACATTCATTTGGATTCCCCTCTTCTTTCTCTTTCTTTTCCCTTTGGGCCCTTA |
| | GCCTTACTATGATGATATGAGA |

| | |
|---|---|
| ORF Start: ATG at 58 | ORF Stop: TGA at 1789 |

| | | |
|---|---|---|
| SEQ ID NO: 60 | 577 aa | MW at 64284.7 kD |

| | |
|---|---|
| NOV19b, | MHWLRKVQGLCTLWGTQMSSRTLYINSRQLVSLQWGHQEVPAKFNFASDVLDHWADME |
| CG125215- | |
| 02 Protein | KAGKRLPSPALWWVNGKGKELMWNFRELSENSRQAANVLSGACGLQRGDRVAVMLPRV |
| Sequence | |
| | PEWWLVILGCIRAGLIFMPGTIQMKSTDILYRLQMSKAKAIVAGDEVIQEVDTVASEC |
| | PSLRIKLLVSEKSCDGWLNFKKLLNEASTTHHCVETGSQEASAIYFTSGTSGLPKMAE |
| | HSYSSLGLKAKMDAGWTGLQASDIMWTISDTGWILNILGSLLESWTLGACTFVHLLPK |
| | FDPLVILKTLSSYPIKSMMGAPIVYRMLLQQDLSSYKFPHLQNCLAGGESLLPETLEN |
| | WRAQTGLDIREFYGQTETGLTCMVSKTMKIKPGYMGTAASCYDVQVIDDKGNVLPPGT |
| | EGDIGIRVKPIRPIGIFSGYVENPDKTAANIRGDFWLLGDRGIKDEDGYFQFMGRADD |
| | IINSSGYRIGPSEVENALMKHPAVVETAVISSPDPVRGEVVKAFVILASQFLSHDPEQ |
| | LTKELQQHVKSVTAPYKYPRKIEFVLNLPKTVTGKIQRTKLRDKEWKMSGKARAQ |

Sequence comparison of the above protein sequences yields the following sequence relationships shown in Table 19B.

TABLE 19B
Comparison of NOV19a against NOV19b.

| | | Identities/ |
|---|---|---|
| Protein | NOV19a Residues/ | Similarities for |
| Sequence | Match Residues | the Matched Region |
| NOV19b | 1 . . . 577 | 575/577 (99%) |
| | 1 . . . 577 | 577/577 (99%) |

Further analysis of the NOV19a protein yielded the following properties shown in Table 19C.

TABLE 19C
Protein Sequence Properties NOV19a

| | |
|---|---|
| PSort | 0.6000 probability located in endoplasmic reticulum |
| analysis: | (membrane); 0.3686 probability located in microbody |
| | (peroxisome); 0.2058 probability located in mitochondrial |
| | inner membrane; 0.1000 probability located in plasma |
| | membrane |
| SignalP | Cleavage site between residues 20 and 21 |
| analysis: | |

A search of the NOV19a protein against the Geneses database, a proprietary database that contains sequences published in patents and patent publication, yielded several homologous proteins shown in Table 19D.

TABLE 19D
Geneseq Results for NOV19a

| | | NOV19a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAB43245 | Human ORFX ORF3009 polypeptide | 41 . . . 577 | 534/537 (99%) | 0.0 |
| | sequence SEQ ID NO: 6018-Homo | 1 . . . 537 | 536/537 (99%) | |
| | sapiens, 537 aa. [WO200058473-A2, | | | |
| | 5 Oct. 2000] | | | |
| AAM41894 | Human polypeptide SEQ ID NO 6825- | 246 . . . 574 | 316/329 (96%) | 0.0 |
| | Homo sapiens, 390 aa. [WO200153312- | 2 . . . 330 | 321/329 (97%) | |
| | A1, 26 Jul. 2001] | | | |
| AAU23625 | Novel human enzyme polypeptide | 263 . . . 577 | 307/315 (97%) | e−179 |
| | #711-Homo sapiens, 315 aa. | 1 . . . 315 | 307/315 (97%) | |
| | [WO200155301-A2, 2 Aug. 2001] | | | |
| AAU23060 | Novel human enzyme polypeptide | 250 . . . 577 | 310/337 (91%) | e−179 |
| | #146-Homo sapiens, 342 aa. | 6 . . . 342 | 313/337 (91%) | |
| | [WO200155301-A2, 2 Aug. 2001] | | | |
| ABB53263 | Human polypeptide #3-Homo sapiens, | 38 . . . 560 | 235/528 (44%) | e−126 |
| | 583 aa. [WO200181363-A1, | 43 . . . 567 | 334/528 (62%) | |
| | 1 Nov. 2001] | | | |

In a BLAST search of public sequence databases, the NOV19a protein was found to have homology to the proteins shown in the BLAST data in Table 19E.

TABLE 19E
Public BLASTP Results for NOV19a

| | | NOV19a | | |
|---|---|---|---|---|
| Protein | | Residues/ | Identities/ | |
| Accession | | Match | Similarities for | Expect |
| Number | Protein/Organism/Length | Residues | the Matched Portion | Value |
| O70490 | Kidney-specific protein-Rattus | 1 . . . 572 | 445/572 (77%) | 0.0 |
| | norvegicus (Rat), 572 aa. | 1 . . . 572 | 507/572 (87%) | |
| AAH31140 | Hypothetical 64.3 kDa protein-Mus | 1 . . . 574 | 437/575 (76%) | 0.0 |
| | musculus (Mouse), 575 aa. | 1 . . . 575 | 502/575 (87%) | |
| Q96LX4 | CDNA FLJ33088 fis, clone | 1 . . . 574 | 437/575 (76%) | 0.0 |
| | TRACH2000496, highly similar to | 1 . . . 575 | 501/575 (87%) | |
| | Rattus norvegicus kidney-specific | | | |
| | protein (KS) mRNA-Homo sapiens | | | |
| | (Human), 575 aa. | | | |
| Q9TVBS | Xenobiotic/medium-chain fatty | 4 . . . 568 | 330/575 (57%) | 0.0 |
| | acid: CoA ligase form XL-III precursor- | 1 . . . 574 | 428/575 (74%) | |
| | Bos taurus (Bovine), 577 aa. | | | |
| Q9BEA2 | Lipoate-activating enzyme precursor- | 4 . . . 568 | 329/575 (57%) | 0.0 |
| | Bos taurus (Bovine), 577 aa. | 1 . . . 574 | 427/575 (74%) | |

PFam analysis predicts that the NOV19a protein contains the domains shown in the Table 19F.

TABLE 19F
Domain Analysis of NOV19a

| | | Identities/ | |
|---|---|---|---|
| Pfam | | Similarities for | Expect |
| Domain | NOV19a Match Region | the Matched Region | Value |
| AMP-binding | 82 . . . 493 | 108/421 (26%) | 2.1e−90 |
| | | 287/421 (68%) | |

### Example 20

The NOV20 clone was analyzed, and the nucleotide and encoded polypeptide sequences are shown in Table 20A.

TABLE 20A
NOV20 Sequence Analysis

| | |
|---|---|
| SEQ ID NO: 61 | 3162 bp |

| | |
|---|---|
| NOV20a, | GAATNTCGCCCTTACACGTAGAGGAGAGAAAAGCGACCAAGATAAAAGTGGACAGAAG |
| CG125332- | |
| 02 DNA | AATAAGCGAGACTTTTTATCCATGAAACAGTCTCCTGCCCTCGCTCCGGAAGAGCGCT |
| Sequence | |
| | GCCGCAGAGCCGGGTCCCCAAAGCCGGTCTTGAGAGCTGATGACAATAACATGGGCAA |
| | TGGCTGCTCTCAGAAGCTGGCGACTGCTAACCTCCTCCGGTTCCTATTGCTGGTCCTG |
| | ATTCCATGTATCTGTGCTCTCGTTCTCTTGCTGGTGATCCTGCTTTCCTATGTTGGAA |
| | CATTACAAAAGGTCTATTTTAAATCAAATGGGAGTGAACCTTTGGTCACTGATGGTGA |
| | AATCCAAGGGTCCGATGTTATTCTTACAAATACAATTTATAACCAGAGCACTGTGGTG |
| | TCTACTGCACATCCCGACCAACACGTTCCAGCCTGGACTACGGATGCTTCTCTCCCAG |
| | GGGACCAAAGTCACAGGAATACAAGTGCCTGTATGAACATCACCCACAGCCAGTGTCA |
| | GATGCTGCCCTACCACGCCACGCTGACACCTCTCCTCTCAGTTGTCAGAAACATGGAA |
| | ATGGAAAAGTTCCTCAAGTTTTTCACATATCTCCATCGCCTCAGTTGCTATCAACATA |
| | TCATGCTGTTTGGCTGTACCCTCGCCTTCCCTGAGTGCATCATTGATGGCGATGACAG |
| | TCATGGACTCCTGCCCTGTAGGTCCTTCTGTGAGGCTGCAAAAGAAGGCTGTGAATCA |
| | GTCCTGGGGATGGTGAATTACTCCTGGCCGGATTTCCTCAGATGCTCCCAGTTTAGAA |
| | ACCAAACTGAAAGCAGCAATGTCAGCAGAATTTGCTTCTCACCTCAGCAGGAAAACGG |
| | AAAGCAATTGCTCTGTGGAAGGGGTGAGAACTTTCTGTGTGCCAGTGGAATCTGCATC |
| | CCCGGGAAACTGCAATGTAATGGCTACAACGACTGTGACGACTGGAGTGACGAGGCTC |
| | ATTGCAACTGCAGCGAGAATCTGTTTCACTGTCACACAGGCAAGTGCCTTAATTACAG |
| | CCTTGTGTGTGATGGATATGATGACTGTGGGGATTTGAGTGATGAGCAAAACTGTGAT |
| | TGCAATCCCACAACAGAGCATCGCTGCGGGGACGGGCGCTGCATCGCCATGGAGTGGG |
| | TGTGTGATGGTGACCACGACTGTGTGGATAAGTCCGACGAGGTCAACTGCTCCTGTCA |
| | CAGCCAGGGTCTGGTGGAATGCAGAAATGGACAATGTATCCCCAGCACGTTTCAATGT |
| | GATGGTGACGAGGACTGCAAGGATGGGAGTGATGAGGAGAACTGCAGCGTCATTCAGA |
| | CTTCATGTCAAGAAGGACACCAAAGATGCCTCTACAATCCCTGCCTTGATTCATGTGG |
| | TGGTAGCTCTCTCTGTGACCCGAACAACAGTCTGAATAACTGTAGTCAATGTGAACCA |
| | ATTACATTGGAACTCTGCATGAATTTGCCCTACAACAGTACAAGTTATCCAAATTATT |
| | TTGGCCACAGGACTCAAAAGGAAGCATCCATCAGCTGGGAGTCTTCTCTTTTCCCTGC |
| | ACTTGTTCAAACCAACTGTTATAAATACCTCATGTTCTTTTCTTGCACCATTTTGGTA |
| | CCAAAATGTGATGTGAATACAGGCGAGCATATCCCTCCTTGCAGGGCATTGTGTGAAC |
| | ACTCTAAAGAACGCTGTGAGTCTGTTCTTGGGATTGTGGGCCTACAGTGGCCTGAAGA |
| | CACAGATTGCAGTCAATTTCCAGAGGAAAATTCAGACAATCAAACCTGCCTGATGCCT |
| | GATGAATATGTGGAAGGTTGTAAAGAGAGAGATCTTTGGGAATGTCCATCCAATAAAC |
| | AATGTTTGAAGCACACAGTGATCTGCGATGGGTTCCCAGACTGCCCTGATTACATGGA |
| | CGAGAAAAACTGCTCATTTTGCCAAGATGATGAGCTGGAATGTGCAAACCATGCGTGT |
| | GTGTCACGTGACCTGTGGTGTGATGGTGAAGCCGACTGCTCAGACAGTTCAGATGAAT |
| | GGGACTGTGTGACCCTCTCTATAAATGTGAACTCCTCTTCCTTTCTGATGGTTCACAG |
| | AGCTGCCACAGAACACCATGTGTGTGCAGATGGCTGGCAGGAGATATTGAGTCAGCTG |
| | GCCTGCAAGCAGATGGGTTTAGGAGAACCATCTGTGACCAAATTGATACAGGAACAGG |
| | AGAAAGAGCCGCGGTGGCTGACATTACACTCCAACTGGGAGAGCCTCAATGGGACCAC |
| | TTTACATGAACTTCTAGTAAATGGGCAGTCTTGTGAGAGCAGAAGTAAAATTTCTCTT |
| | CTGTGTACTAAACAAGACTGTGGGCGCCGCCCTGCTGCCCGAATGAACAAAAGGATCC |
| | TTGGAGGTCGGACGAGTCGCCCTGGAAGGTGGCCATGGCAGTGTTCTCTGCAGAGTGA |
| | ACCCAGTGGACATATCTGTGGCTGTGTCCTCATTGCCAAGAAGTGGGTTCTGACAGTT |
| | GCCCACTGCTTCGAGGGGAGAGAGAATGCTGCAGTTTGGAAAGTGGTGCTTGGCATCA |
| | ACAATCTAGACCATCCATCAGTGTTCATGCAGACACGCTTTGTGAAGACCATCATCCT |
| | GCATCCCCGCTACAGTCGAGCAGTGGTGGACTATGACATCAGCATCGTTGGGCTGAGT |
| | GAAGACATCAGTGAGACTGGCTACGTCCGGCCTGTCTGCTTGCCCAACCCGGAGCAGT |
| | GGCTAGAGCCTGACACGTACTGCTATATCACAGGCTGGGGCCACATGGGCAATAAAAT |
| | GCCATTTAAGCTGCAAGAGGGAGAGGTCCGCATTATTTCTCTGGAACATTGTCAGTCC |
| | TACTTTGACATGAAGACCATCACCACTCGGATGATATGTGCTGGCTATGAGTCTGGCA |
| | CAGTTGATTCATGCATGGGTGACAGCGGTGGGCCTCTTGTTTGTGAGAAGCCTGGAGG |
| | ACGGTGGACATTATTTGGATTAACTTCATGGGGCTCCGTCTGCTTTTCCAAAGTCCTG |
| | GGGCCTGGCGTTTATAGTAATGTGTCATATTTCGTCGAATGGATTAAAAGACAGATTT |
| | ACATCCAGACCTTTCTCCTAAACTAATTATAAGGATGATCAGAGACTTTTGCCAGCTA |
| | CACTAAAAGAAAATGGCCTTCTTGACTGTG |

| | |
|---|---|
| ORF Start: ATG at 80 | ORF Slop: TAA at 3098 |

| | | |
|---|---|---|
| SEQ ID NO: 62 | 1006 aa | MW at 112463.8 kD |

| | |
|---|---|
| NOV20a, | MKQSPALAPEERCRRAGSPKPVLRADDNNMGNGCSQKLATANLLRFLLLVLIPCICAL |
| CG125332- | |
| 02 Protein | VLLLVILLSYVGTLQKVYFKSNGSEPLVTDGEIQGSDVILTNTIYNQSTVVSTAHPDQ |
| Sequence | |
| | HVPAWTTDASLPGDQSHRNTSACMNITHSQCQMLPYHATLTPLLSVVRNMEMEKFLKF |
| | FTYLHRLSCYQHIMLFGCTLAFPECIIDGDDSHGLLPCRSFCEAAKEGCESVLGMVNY |
| | SWPDFLRCSQFRNQTESSNVSRICFSPQQENGKQLLCGRGENFLCASGICIPGKLQCN |
| | GYNDCDDWSDEAHCNCSENLFHCHTGKCLNYSLVCDGYDDCGDLSDEQNCDCNPTTEH |
| | RCGDGRCIAMEWVCDGDHDCVDKSDEVNCSCHSQGLVECRNGQCIPSTFQCDGDEDCK |
| | DGSDEENCSVIQTSCQEGDQRCLYNPCLDSCGGSSLCDPNNSLNNCSQCEPITLELCM |
| | NLPYNSTSYPNYFGHRTQKEASISWESSLFPALVQTNCYKYLMFFSCTILVPKCDVNT |
| | GEHIPPCRALCEHSKERCESVLGIVGLQWPEDTDCSQFPEENSDNQTCLMPDEYVEGC |
| | KERDLWECPSNKQCLKHTVICDGFPDCPDYMDEKNCSFCQDDELECANHACVSRDLWC |
| | DGEADCSDSSDEWDCVTLSTNVNSSSFLMVHRAATEHHVCADGWQEILSQLACKQMGL |
| | GEPSVTKLIQEQEKERRWLTLHSNWESLNGTTLHELLVNGQSCESRSKISLLCTKQDC |
| | GRRPAARMNKRILGGRTSRPGRWPWQCSLQSEPSGHICGCVLIAKKWVLTVAHCFEGR |
| | ENAAVWKVVLGINNLDHPSVFMQTRFVKTIILHPRYSRAVVDYDISIVGLSEDISETG |
| | YVRPVCLPNPEQWLEPDTYCYITGWGHMGNKMPFKLQEGEVRIISLEHCQSYFDMKTI |
| | TTRMICAGYESGTVDSCMGDSGGPLVCEKPGGRWTLFGLTSWGSVCFSKVLGPGVYSN |
| | VSYFVEWIKRQIYIQTFLLN |

Further analysis of the NOV20a protein yielded the following properties shown in Table 20B.

TABLE 20B
Protein Sequence Properties NOV20a

| | |
|---|---|
| PSort | 0.9000 probability located in Golgi body; 0.7900 probability |
| analysis: | located in plasma membrane; 0.2000 probability located in |
| | endoplasmic reticulum (membrane); 0.1000 probability |
| | located in mitochondrial inner membrane |
| SignalP | Cleavage site between residues 68 and 69 |
| analysis: | |

A search of the NOV20a protein against the Geneseq database, a proprietary database that contains sequences published in patents and patent publication, yielded several homologous proteins shown in Table 20C.

TABLE 20C
Geneseq Results for NOV20a

| | | NOV20a | | |
|---|---|---|---|---|
| | Protein/Organism/ | Residues/ | Identities | |
| Geneseq | Length [Patent #, | Match | Similarities for | Expect |
| Identifier | Date] | Residues | the Matched Region | Value |
| ABB11975 | Human corin homologue, SEQ ID | 1 . . . 1006 | 1004/1042 (96%) | 0.0 |
| | NO: 2345-Homo sapiens, 1076 aa. | 35 . . . 1076 | 1004/1042 (96%) | |
| | [WO200157188-A2, 9 Aug. 2001] | | | |
| AAE06939 | Human corin protein-Homo sapiens, | 1 . . . 1006 | 1003/1042 (96%) | 0.0 |
| | 1042 aa. [WO200157194-A2, | 1 . . . 1042 | 1003/1042 (96%) | |
| | 9 Aug. 2001] | | | |
| AAY44426 | Human serine protease, Corin-Homo | 1 . . . 1006 | 1003/1042 (96%) | 0.0 |
| | sapiens, 1042 aa. [WO9964608-A1, | 1 . . . 1042 | 1003/1042 (96%) | |
| | 16 Dec. 1999] | | | |
| AAY44427 | Mouse Serine protease, Corin-Mus | 13 . . . 1004 | 820/1029 (79%) | 0.0 |
| | musculus, 1113 aa. [WO9964608-A1, | 81 . . . 1107 | 890/1029 (85%) | |
| | 16 Dec. 1999] | | | |
| AAW46917 | Amino acid sequence of a novel | 656 . . . 1006 | 348/351 (99%) | 0.0 |
| | human kallikrein-Homo sapiens, | 6 . . . 356 | 348/351 (99%) | |
| | 356 aa. [WO9803665-A1, | | | |
| | 29 Jan. 1998] | | | |

In a BLAST search of public sequence databases, the NOV20a protein was found to have homology to the proteins shown in the BLASTP data in Table 20D.

TABLE 20D
Public BLASTP Results for NOV20a

| | | NOV20a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9Y5Q5 | Atrial natriuteric peptide-converting | 1 . . . 1006 | 1003/1042 (96%) | 0.0 |
| | enzyme (EC 3.4.21.-) (pro-ANP- | 1 . . . 1042 | 1003/1042 (96%) | |
| | converting enzyme) (Corin) (Heart | | | |
| | specific serine proteinase ATC2)-Homo | | | |
| | sapiens (Human), 1042 aa. | | | |
| Q9Z319 | Atrial natriuteric peptide-converting | 13 . . . 1004 | 817/1029 (79%) | 0.0 |
| | enzyme (EC 3.4.21.-) (pro-ANP- | 81 . . . 1107 | 887/1029 (85%) | |
| | converting enzyme) (Corin) (Low density | | | |
| | lipoprotein receptor related protein 4)- | | | |
| | Mus musculus (Mouse), 1113 aa. | | | |
| Q9V4N6 | CG2105 protein-Drosophila | 455 . . . 998 | 191/575 (33%) | 9e−85 |
| | melanogaster (Fruit fly), 1379 aa. | 761 . . . 1329 | 286/575 (49%) | |
| Q95LS5 | Hypothetical 14.8 kDa protein-Macaca | 140 . . . 268 | 122/129 (94%) | 2e−69 |
| | fascicularis (Crab eating macaque) | 1 . . . 129 | 126/129 (97%) | |
| | (Cynomolgus monkey), 129 aa. | | | |
| P98072 | Enteropeptidase precursor (EC 3.4.21.9) | 619 . . . 995 | 137/387 (35%) | 2e−61 |
| | (Enterokinase)-Bos taurus (Bovine). | 659 . . . 1031 | 206/387 (52%) | |
| | 1035 aa. | | | |

PFam analysis predicts that the NOV20a protein contains the domains shown in the Table 20E.

TABLE 20E
Domain Analysis of NOV20a

| | | Identities/ | |
|---|---|---|---|
| Pfam | | Similarities for | Expect |
| Domain | NOV20a Match Region | the Matched Region | Value |
| Fz | 129 . . . 257 | 42/153 (27%) | 6.9e−39 |
| | | 105/153 (69%) | |
| ldl_recept_a | 267 . . . 304 | 19/44 (43%) | 9.3e−08 |
| | | 32/44 (73%) | |
| ldl_recept_a | 305 . . . 342 | 18/43 (42%) | 7.9e−10 |
| | | 30/43 (70%) | |
| ldl_recept_a | 344 . . . 379 | 21/43 (49%) | 8.3e−10 |
| | | 30/43 (70%) | |
| ldl_recept_a | 385 . . . 416 | 19/43 (44%) | 2.9e−09 |
| | | 28/43 (65%) | |
| Fz | 445 . . . 571 | 54/150 (36%) | 2.2e−52 |
| | | 108/150 (72%) | |
| ldl_recept_a | 578 . . . 618 | 16/43 (37%) | 0.0046 |
| | | 25/43 (58%) | |
| ldl_recept_a | 619 . . . 655 | 16/43 (37%) | 0.00099 |
| | | 28/43 (65%) | |
| Trypsin | 766 . . . 994 | 101/263 (38%) | 2e−75 |
| | | 179/263 (68%) | |

### Example 21

The NOV21 clone was analyzed, and the nucleotide and encoded polypeptide sequences are shown in Table 21A.

TABLE 21A
NOV21 Sequence Analysis

| | |
|---|---|
| SEQ ID NO: 63 | 4840 bp |

| | |
|---|---|
| NOV 21a, | CGCTCTCCCCGCCCCCTCCCTCCCTCGCAGGGGCCGAGCGAATGTAGCCCGCGAGAGA |
| CG125363- | |
| 01 DNA | AAATGGCGGCGGCGGCGGGGAATCGCGCCTCGTCGTCGGGATTCCCGGGCGCCAGGGC |
| Sequence | |
| | TACGAGCCCTGAGCAGCGCGGCGGAGAGGCCCTCAAGGCGAGCAGCGCGCCCGCGGCT |
| | GCCGCGGGACTGCTGCGGGAGGCGGGCAGCGGGGTGCCTGGCGAGCGGGCGGACTGGC |
| | GGCGGCGGCAGCTGCGCAAAGTGCGGAGTGTGGAGCTGGACCAGCTGCCTGAGCAGCC |
| | GCTCTTCCTTGCCGCCTCACCGCCGGCCTCCTCGACTTCCCCGTCGCCGGAGCCCGCG |
| | GACGCAGCGGGGAGTGGGACCGGCTTCCAGCCTGTGGCGGTGCCGCCGCCCCACGGAG |
| | CCGCCAGCCGCGGCGGCGCCCACCTTACCGAGTCGGTGGCGGCGCCGGACAGCGGCGC |
| | CTCGAGTCCCGCAGCGGCCGAGCCCGGGGAGAAGCGGGCGCCCGCCGCCGAGCCGTCT |
| | CCTGCAGCGGCCCCCGCCGGTCGTGAGATGGAGAATAAAGAAACTCTCAAAGGGTTGC |
| | ACAAGATGGATGATCGTCCAGAGGAACGAATGATCAGGGAGAAACTGAAGGCAACCTG |
| | TATGCCAGCCTGGAAGCACGAATGGTTGGAAAGGAGAAATAGGCGAGGGCCTGTGGTG |
| | GTAAAACCAATCCCAGTTAAAGGAGATGGATCTGAAATGAATCACTTAGCAGCTGAGT |
| | CTCCAGGAGAGGTCCAGGCAAGTGCGGCTTCACCAGCTTCCAAAGGCCGACGCAGTCC |
| | TTCTCCTGGCAACTCCCCATCAGGTCGCACAGTGAAATCAGAATCTCCAGGAGTAAGG |
| | AGAAAAAGAGTTTCCCCAGTGCCTTTTCAGAGTGGCAGAATCACACCACCCCGAAGAG |
| | CCCCTTCACCAGATGGCTTCTCACCATATAGCCCTGAGGAAACAAACCGCCGTGTTAA |
| | CAAAGTGATGCGGGCCAGACTGTACTTACTGCAGCAGATAGGGCCTAACTCTTTCCTG |
| | ATTGGAGGAGACAGCCCAGACAATAAATACCGGGTGTTTATTGGGCCTCAGAACTGCA |
| | GCTGTGCACGTGGAACATTCTGTATTCATCTGCTATTTGTGATGCTCCGGGTGTTTCA |
| | ACTAGAACCTTCAGACCCAATGTTATGGAGAAAAACTTTAAAGAATTTTGAGGTTGAG |
| | AGTTTGTTCCAGAAATATCACAGTAGGCGTAGCTCAAGGATCAAAGCTCCATCTCGTA |
| | ACACCATCCAGAAGTTTGTTTCACGCATGTCAAATTCTCATACATTGTCATCATCTAG |
| | TACTTCTACATCTAGTTCAGAAAACAGCATAAAGGATGAAGAGGAACAGATGTGTCCT |
| | ATTTGCTTGTTGGGCATGCTTGATGAAGAAAGTCTTACAGTGTGTGAAGACGGCTGCA |
| | GGAACAAGCTGCACCACCACTGCATGTCAATTTGGGCAGAAGAGTGTAGAAGAAATAG |
| | AGAACCTTTAATATGTCCCCTTTGTAGATCTAAGTGGAGATCTCATGATTTCTACAGC |
| | CACGAGTTGTCAAGTCCTGTGGATTCCCCTTCTTCCCTCAGAGCTGCACAGCAGCAAA |
| | CCGTACAGCAGCAGCCTTTGGCTGGATCACGAAGGAATCAAGAGAGCAATTTTAACCT |
| | TACTCATTATGGAACTCAGCAAATCCCTCCTGCTTACAAAGATTTAGCTGAGCCATGG |
| | ATTCAGGTGTTTGGAATGGAACTCGTTGGCTGCTTATTTTCTAGAAACTGGAATGTGA |
| | GAGAGATGGCCCTCAGGCGTCTTTCCCATGATGTCAGTGGGGCCCTGCTGTTGGCAAA |
| | TGGGGAGAGCACTGGAAATTCTGGGGGCAGCAGTGGAAGCAGCCCGAGTGGGGGAGCC |
| | ACCAGTGGGTCTTCCCAGACCAGTATCTCAGGAGATGTGGTGGAGGCATGCTGCAGCG |
| | TTCTGTCAATGGTCTGTGCTGACCCTGTCTACAAAGTGTACGTTGCTGCTTTAAAAAC |
| | ATTGAGAGCCATGCTGGTATATACTCCTTGCCACAGTTTAGCGGAAAGAATCAAACTT |
| | CAGAGACTTCTCCAGCCAGTTGTAGACACCATCCTAGTCAAATGTGCAGATGCGAATA |
| | GCCGCACAAGTCAGCTGTCCATATCAACACTGTTGGAACTGTGCAAAGGCCAAGCAGG |
| | AGAGTTGGCAGTTGGCAGAGAAATACTAAAAGCTGGATCCATTGGTATTGGTGGTGTT |
| | GATTATGTCTTAAATTGTATTCTTGGAAACCAAACTGAATCAAACAATTGGCAAGAAC |
| | TTCTTGGCCGCCTTTCTCTTATAGATAGACTGTTGTTGGAATTTCCTGCTGAATTTTA |
| | TCCTCATATTGTCAGTACTGATGTTTCACAAGCTGAGCCTGTTGAAATCAGGTATAAG |
| | AAGCTGCTGTCCCTCTTAACCTTTGCTTTGCAGTCCATTGATAATTCCCACTCAATGG |
| | TTGGCAAACTTTCCAGAAGGATCTACTTGAGTTCTGCAAGAATGGTTACTACAGTACC |
| | CCATGTGTTTTCAAAACTGTTAGAAATGCTGAGTGTTTCCAGTTCCACTCACTTCACC |
| | AGGATGCGTCGCCGTTTGATGGCTATTGCAGATGAGGTCGAAATTGCCGAAGCCATCC |
| | AGTTGGGCGTAGAAGACACTTTGGATGGTCAACAGGACAGCTTCTTGCAGGCATCTGT |
| | TCCCAACAACTATCTGGAAACCACAGAGAACAGTTCCCCTGAGTGCACAGTCCATTTA |
| | GAGAAAACTGGAAAAGGATTATGTGCTACAAAATTGAGTGCCAGTTCAGAGGACATTT |
| | CTGAGAGACTGGCCAGGATTTCAGTAGGACCTTCTAGTTCAACAACAACAACAACAAC |
| | AACAACAGAGCAACCAAAGCCAATGGTTCAAACAAAAGGCAGACCCCACAGTCAGTGT |
| | TTGAACTCCTCTCCTTTATCTCATCATTCCCAATTAATGTTTCCAGCCTTGTCAACCC |
| | CTTCTTCTTCTACCCCATCTGTACCAGCTGGCACTGCAACAGATGTCTCTAAGCATAG |
| | ACTTCAGGGATTCATTCCCTGCAGAATACCTTCTGCATCTCCTCAAACACAGCGCAAG |
| | TTTTCTCTACAATTCCACAGAAACTGTCCTGAAAACAAAGACTCAGATAAACTTTCCC |
| | CAGTCTTTACTCAGTCAAGACCCTTGCCCTCCAGTAACATACACAGGCCAAAGCCATC |
| | TCGACCTACCCCAGGTAATACAAGTAAACAGGGAGATCCCTCAAAAAATAGCATGACA |
| | CTTGATCTGAACAGTAGTTCCAAATGTGATGACAGCTTTGGCTGTAGCAGCAATAGTA |
| | GTAATGCTGTTATACCCAGTGACGAGACAGTGTTCACCCCAGTAGAGGAGAAATGCAG |
| | ATTAGATGTCAATACAGAGCTCAACTCCAGTATTGAGGACCTTCTTGAAGCATCTATG |
| | CCTTCAAGTGATACAACAGTAACTTTTAAGTCAGAAGTTGCTGTCCTGTCTCCTGAAA |
| | AGGCTGAAAATGATGATACCTACAAAGATGATGTGAATCATAATCAAAAGTGCAAAGA |
| | GAAGATGGAAGCTGAAGAAGAAGAAGCTTTAGCAATTGCCATGGCAATGTCAGCGTCT |
| | CAGGATGCCCTCCCCATAGTTCCTCAGCTGCAGGTTGAAAATGGAGAAGATATCATCA |
| | TTATTCAACAGGATACACCAGAGACTCTACCAGGACATACCAAAGCAAAACAACCGTA |
| | TAGAGAAGACACTGAATGGCTGAAAGGTCAACAGATAGGCCTTGGAGCATTTTCTTCT |
| | TGTTATCAGGCTCAAGATGTGGGAACTGGAACTTTAATGGCTGTTAAACAGGTGACTT |
| | ATGTCAGAAACACATCTTCTGAGCAAGAAGAAGTAGTAGAAGCACTAAGAGAAGAGAT |
| | AAGAATGATGAGCCATCTGAATCATCCAAACATCATTAGGATGTTGGGAGCCACGTGT |
| | GAGAAGAGCAATTACAATCTCTTCATTGAATGGATGGCAGGGGGATCGGTGGCTCATT |
| | TGCTGAGTAAATATGGAGCCTTCAAAGAATCAGTAGTTATTAACTACACTGAACAGTT |
| | ACTCCGTGGCCTTTCGTATCTCCATGAAAACCAAATCATTCACAGAGATGTCAAAGGT |
| | GCCAATTTGCTAATTGACAGCACTGGTCAGAGACTAAGAATTGCAGATTTTGGAGCTG |
| | CAGCCAGGTTGGCATCAAAAGGAACTGGTGCAGGAGAGTTTCAGGGACAATTACTGGG |
| | GACAATTGCATTTATGGCACCTGAGGTACTAAGAGGTCAACAGTATGGAAGGAGCTGT |
| | GATGTATGGAGTGTTGGCTGTGCTATTATAGAAATGGCTTGTGCAAAACCACCATGGA |
| | ATGCAGAAAAACACTCCAATCATCTTGCTTTGATATTTAAGATTGCTAGTGCAACTAC |
| | TGCTCCATCGATCCCTTCACATTTGTCTCCTGGTTTACGAGATGTGGCTCTTCGTTGT |
| | TTAGAACTTCAACCTCAGGACAGACCTCCATCAAGAGAGCTACTGAAGCATCCAGTCT |
| | TTCGTACTACATGGTAGCCAATTATGCAGATCAACTACAGTAGAAACAGGATGCTCAA |
| | CAAGAGAAAAAAAACTTGTGGGGAACCACATTGATATTCTACTGGCCATGATGCCACT |
| | GAACAGCTATGAACGAGGCCAGTGGGGAACCCTTACCTAAGTATGTGATTGACAAATC |
| | ATGATCTGTACCTAAGCTCAGTATGCAAAAGCCCAAACTAGTGCAGAAACTGTAAACT |

| | |
|---|---|
| ORF Start: ATG at 61 | ORF Stop: TAG at 4597 |

| | | |
|---|---|---|
| SEQ ID NO: 64 | 1512 aa | MW at 164748.2 kD |

| | |
|---|---|
| NOV21a, | MAAAAGNRASSSGFPGARATSPEQRGGEALKASSAPAAAAGLLREAGSGVPGERADWR |
| CG125363- | |
| 01 Protein | RRQLRKVRSVELDQLREQPLFLAASPPASSTSPSPEPADAAGSGTGFQPVAVPPPHGA |
| Sequence | |
| | ASRGGAHLTESVAAPDSGASSPAAAEPGEKRAPAAEPSPAAAPAGREMENKETLKGLH |
| | KMDDRPEERMIREKLKATCMPAWKHEWLERRNRRGPVVVKPIPVKGDGSEMNHLAAES |
| | PGEVQASAASPASKGRRSPSPGNSPSGRTVKSESPGVRRKRVSPVPFQSGRITPPRRA |
| | RSPDGFSPYSPEETNRRVNKVMRARLYLLQQIGPNSFLIGGDSPDNKYRVFIGPQNCS |
| | CARGTFCIHLLFVMLRVFQLEPSDPMLWRKTLKNFEVESLFQKYHSRRSSRIKAPSRN |
| | TIQKFVSRMSNSHTLSSSSTSTSSSENSIKDEEEQMCPICLLGMLDEESLTVCEDGCR |
| | NKLHHHCMSIWAEECRRNREPLICPLCRSKWRSHDFYSHELSSPVDSPSSLRAAQQQT |
| | VQQQPLAGSRRNQESNFNLTHYGTQQIPPAYKDLAEPWIQVFGMELVGCLFSRNWNVR |
| | EMALRRLSHDVSGALLLANGESTGNSGGSSGSSPSGGATSGSSQTSISGDVVEACCSV |
| | LSMVCADPVYKVYVAALKTLRAMLVYTPCHSLAERIKLQRLLQPVVDTILVKCADANS |
| | RTSQLSISTLLELCKGQAGELAVGREILKAGSIGIGGVDYVLNCILGNQTESNNWQEL |
| | LGRLCLIDRLLLEFPAEFYPHIVSTDVSQAEPVEIRYKKLLSLLTFALQSIDNSHSMV |
| | GKLSRRIYLSSARMVTTVPHVFSKLLEMLSVSSSTHFTRMRRRLMAIADEVEIAEAIQ |
| | LGVEDTLDGQQDSFLQASVPNNYLETTENSSPECTVHLEKTGKGLCATKLSASSEDIS |
| | ERLARISVGPSSSTTTTTTTTEQPKPMVQTKGRPHSQCLNSSPLSHHSQLMFPALSTP |
| | SSSTPSVPAGTATDVSKHRLQGFIPCRIPSASPQTQRKPSLQFHRNCPENKDSDKLSP |
| | VFTQSRPLPSSNIHRPKPSRPTPGNTSKQGDPSKNSMTLDLNSSSKCDDSFGCSSNSS |
| | NAVIPSDETVFTPVEEKCRLDVNTELNSSIEDLLEASMPSSDTTVTFKSEVAVLSPEK |
| | AENDDTYKDDVNHNQKCKEKMEAEEEEALAIAMAMSASQDALPIVPQLQVENGEDIII |
| | IQQDTPETLPGHTKAKQPYREDTEWLKGQQIGLGAFSSCYQAQDVGTGTLMAVKQVTY |
| | VRNTSSEQEEVVEALREEIRMMSHLNHPNIIRMLGATCEKSNYNLFIEWMAGGSVAHL |
| | LSKYGAFKESVVINYTEQLLRGLSYLHENQIIHRDVKGANLLIDSTGQRLRIADFGAA |
| | ARLASKGTGAGEFQGQLLGTIAFMAPEVLRGQQYGRSCDVWSVGCAIIEMACAKPPWN |
| | AEKHSNHLALIFKIASATTAPSIPSHLSPGLRDVALRCLELQPQDRRPSRELLKHPVF |
| | RTTW |

Further analysis of the NOV21a protein yielded the following properties shown in Table 21B.

TABLE 21B
Protein Sequence Properties NOV21a

| | |
|---|---|
| PSort | 0.8800 probability located in nucleus; 0.4689 probability |
| analysis: | located in mitochondrial matrix space: 0.3000 probability |
| | located in microbody (peroxisome); 0.1702 probability located |
| | in mitochondrial inner membrane |
| SignalP | No Known Signal Sequence Predicted |
| analysis: | |

A search of the NOV2a protien against the Geneseq database, a proprietor, database that contains sequences published in patents and patent publication, yielded several homologous proteins shown in Table 21C.

TABLE 21C
Geneseq Results for NOV21a

| | | NOV21a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| ABG04377 | Novel human diagnostic protein | 21 . . . 1512 | 1456/1495 (97%) | 0.0 |
| | #4368-Homo sapiens, 1495 aa. | 2 . . . 1495 | 1459/1495 (97%) | |
| | [WO200175067-A2, 11 Oct. 2001] | | | |
| AAG80184 | Human MEK kinase MEKK1 protein | 21 . . . 1512 | 1456/1495 (97%) | 0.0 |
| | fragment-Homo sapiens, 1495 aa. | 2 . . . 1495 | 1459/1495 (97%) | |
| | [WO200179501-A2, 25 Oct. 2001] | | | |
| AAB60291 | Human MEKK1-Homo sapiens, | 21 . . . 1512 | 1456/1495 (97%) | 0.0 |
| | 1495 aa. [U.S. Pat. No. 6168950-B1, | 2 . . . 1495 | 1459/1495 (97%) | |
| | 2 Jan. 2001] | | | |
| ABG04377 | Novel human diagnostic protein | 21 . . . 1512 | 1456/1495 (97%) | 0.0 |
| | #4368-Homo sapiens, 1495 aa. | 2 . . . 1495 | 1459/1495 (97%) | |
| | [WO200175067-A2, 11 Oct. 2001] | | | |
| ABG01872 | Novel human diagnostic protein | 46 . . . 1419 | 1342/1376 (97%) | 0.0 |
| | #1863-Homo sapiens, 1375 aa. | 1 . . . 1375 | 1345/1376 (97%) | |
| | [WO200175067-A2, 11 Oct. 2001] | | | |

In a BLAST search of public sequence databases, the NOV21a protein was found to have homology to the proteins shown in the BLASTP data in Table 21D.

TABLE 21D
Public BLASTP Results for NOV21a

| | | NOV21a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q13233 | Mitogen-activated protein kinase kinase | 21 . . . 1512 | 1456/1495 (97%) | 0.0 |
| | kinase 1 (EC 2.7.1.-) (MAPK/ERK | 2 . . . 1495 | 1459/1495 (97%) | |
| | kinase kinase 1) (MEK kinase 1) | | | |
| | (MEKK 1)-Homo sapiens (Human), | | | |
| | 1495 aa (fragment). | | | |
| P53349 | Mitogen-activated protein kinase kinase | 1 . . . 1512 | 1354/1519 (89%) | 0.0 |
| | kinase 1 (EC 2.7.1.-) (MAPK/ERK | 1 . . . 1493 | 1400/1519 (92%) | |
| | kinase kinase 1) (MEK kinase 1) | | | |
| | (MEKK 1)-Mus musculus (Mouse), | | | |
| | 1493 aa. | | | |
| Q62925 | Mitogen-activated protein kinase kinase | 1 . . . 1512 | 1343/1514 (88%) | 0.0 |
| | kinase 1 (EC 2.7.1.-) (MAPK/ERK | 1 . . . 1493 | 1387/1514 (90%) | |
| | kinase kinase 1) (MEK kinase 1) | | | |
| | (MEKK 1)-Rattus norvegicus (Rat), | | | |
| | 1493 aa. | | | |
| A46212 | MEK kinase-mouse, 687 aa. | 811 . . . 1512 | 628/702 (89%) | 0.0 |
| | | 1 . . . 687 | 649/702 (91%) | |
| A48084 | STE11 protein kinase homolog NPK1- | 1227 . . . 1506 | 121/288 (42%) | 6e−59 |
| | common tobacco, 706 aa. | 74 . . . 356 | 181/288 (62%) | |

PFam analysis predicts that the NOV21a protein contains the domains shown in the Table 21E.

TABLE 21E
Domain Analysis of NOV21a

| | | Identities/ | |
|---|---|---|---|
| Pfam | | Similarities for | Expect |
| Domain | NOV21a Match Region | the Matched Region | Value |
| PHD | 442 . . . 494 | 13/53 (25%) | 0.3 |
| | | 31/53 (58%) | |
| Pkinase | 1243 . . . 1508 | 87/305 (29%) | 5.9e−81 |
| | | 210/305 (69%) | |

### Example 22

The NOV22 clone was analyzed, and the nucleotide and encoded polypeptide sequences are shown in Table 22A.

TABLE 22A
NOV22 Sequence Analysis

| | |
|---|---|
| SEQ ID NO:65 | 2121 bp |

| | |
|---|---|
| NOV22a, | GGAGGAAGCGTGGAAATGTGCTTCCGGACAAAGCTCTCAGTATCCTGGGTGCCATTGT |
| CG126012- | |
| 01 DNA | TTCTTCTACTCAGCCGTGTTTTTTCTACTGAGACAGACAAACCCTCAGCCCAGGATAG |
| Sequence | |
| | CAGAAGCCGTGGGAGTTCAGGCCAACCGGCAGACCTGCTACAGGTTCTCTCTGCTGGT |
| | GACCACCCACCCCACAACCACTCAAGAAGCCTCATCAAAACATTGTTGGAGAAAACTG |
| | GGTGCCCACGGAGGAGAAACGGAATGCAAGGAGATTGCAATCTGTGTTTTCTTCCACA |
| | GTGCTTTGAACCAGATGCACTATTACTAATAGCTGGAGGAAATTTTGAAGATCAGCTT |
| | AGAGAAGAAGTGGTCCAGAGAGTTTCTCTTCTCCTTCTCTATTACATTATTCATCAGG |
| | AAGAGATCTGTTCTTCAAAGCTCAACATGAGTAATAAAGAGTATAAATTTTACCTACA |
| | CAGCCTACTGAGCCTCAGGCAGGATGAAGATTCCTCTTTCCTTTCACAGAATGAGACA |
| | GAAGATATCTTGGCTTTCACCAGCCAGTACTTTGACACTTCTCAAAGCCAGTGTATGG |
| | AAACCAAAACGCTGCAGAAAAAATCTGGAATAGTGAGCAGTGAAGGTGCTAATGAAAG |
| | TACGCTTCCTCAGTTGGCAGCCATGATCATTACTTTGTCCCTCCAGGGTGTTTGTCTG |
| | GGACAAGGAAACTTGCCTTCCCCAGACTACTTTACAGAATATATTTTCAGTTCCTTGA |
| | ATCGTACGAATACCCTCCGCCTATCAGAACTAGACCAACTCCTCAACACTCTCTGGAC |
| | CAGAAGTACTTGTATCAAAAATGAGAAAATCCATCAATTTCAAAGGAAACAAAACAAC |
| | ATAATAACCCATGATCAGGACTATTCTAATTTCTCTTCATCCATGGAAAAAGAGTCTG |
| | AGGATGGTCCAGTTTCCTGGGATCAGACCTGCTTCTCTGCTAGGCAGCTGGTGGAGAT |
| | ATTTCTACAGAAGGGCCTCTCACTCATTTCTAAGGAGGACTTTAAGCAAATGAGTCCA |
| | GGGATCATCCAGCAGCTCCTCAGCTGCTCCTGCCACTTACCCAAGGACCAACAAGCAA |
| | AGCTGCCACCTACCACTCTGGAGGAATACGGCTACAGCACGGTGGCTGTCACCCTTCT |
| | CACACTGGGCTCCATGCTGGGGACAGCGCTGGTCCTTTTCCATAGCTGTGAGGAGAAC |
| | TACAGGCTTATCTTACAGCTGTTTGTGGGCTTGGCCGTCGGGACACTGTCTGGGGACG |
| | CTCTGCTCCACCTTATCCCTCAGGTACTTGGTTTACATAAGCAGGAAGCCCCAGAATT |
| | TGGGCATTTCCATGAAAGCAAAGGTCATATTTGGAAACTGATGGGATTAATTGGAGGC |
| | ATCCATGGATTTTTCTTGATAGAAAAATGTTTTATTCTTCTTGTATCACCAAATGACA |
| | AGAAAAGCCCAGAAGATTCACAGGCAGCTGAAATGCCTATAGGCAGTATGACAGCCTC |
| | CAACAGAAAATGTAAAGCCATTAGCTTGTTAGCAATCATGATTCTGGTTGGGGACAGC |
| | CTGCATAATTTTGCAGATGGCCTAGCCATAGGAGCAGCCTTCTCATCATCATCCGAGT |
| | CAGGAGTGACCACTACGATTGCTATCTTGTGTCATGAAATCCCACATGAAATGGGAGA |
| | CTTTGCCGTGCTCTTAAGCTCTGGACTTTCTATGAAGACTGCCATCCTGATGAATTTT |
| | ATAAGCTCCCTAACTGCCTTCATGGGATTATACATTGGCCTTTCCGTGTCAGCTGATC |
| | CATGTGTTCAAGACTGGATCTTCACAGTCACTGCTGGGATGTTCTTATATTTATCCTT |
| | GGTTGAAATGCCTGAAATGACTCATGTTCAAACACAACGACCCTGGATGATGTTTCTC |
| | CTGCAAAACTTTGGATTGATCCTAGGTTGGCTTTCTCTCCTGCTCTTGGCTATATATG |
| | AGCAAAATATTAAAATATAAGTGAGGATCTTCAACATCTTTCAAAAATGCATTTATAT |
| | AGTCTTACTTTGTTTCTTTCATTGCACTCTATAATGATTTTTAAATTAAGAATTTTTT |
| | ATCTTAGGCAAAGTGTGTCTCTTTCAATTCATT |

| | |
|---|---|
| ORF Start: ATG at 16 | ORF Stop: TAA at 1990 |

| | | |
|---|---|---|
| SEQ ID NO: 66 | 658 aa | MW at 73339.6 kD |

| | |
|---|---|
| NOV22a, | MCFRTKLSVSWVPLFLLLSRVFSTETDKPSAQDSRSRGSSGQPADLLQVLSAGDHPPH |
| CG126012- | |
| 01 Protein | NHSRSLIKTLLEKTGCPRRRNGMQGDCNLCFLPQCFEPDALLLIAGGNFEDQLREEVV |
| Sequence | |
| | QRVSLLLLYYIIHQEEICSSKLNMSNKEYKFYLHSLLSLRQDEDSSFLSQNETEDILA |
| | FTRQYFDTSQSQCMETKTLQKKSGIVSSEGANESTLPQLAAMIITLSLQGVCLGQGNL |
| | PSPDYFTEYIFSSLNRTNTLRLSELDQLLNTLWTRSTCIKNEKIHQFQRKQNNIITHD |
| | QDYSNFSSSMEKESEDGPVSWDQTCFSARQLVEIFLQKGLSLISKEDFKQMSPGIIQQ |
| | LLSCSCHLPKDQQAKLPPTTLEEYGYSTVAVTLLTLGSMLGTALVLFHSCEENYRLIL |
| | QLFVGLAVGTLSGDALLHLIPQVLGLHKQEAPEFGHFHESKGHIWKLMGLIGGIHGFF |
| | LIEKCFILLVSPNDKKSPEDSQAAEMPIGSMTASNRKCKAISLLAIMILVGDSLHNFA |
| | DGLAIGAAFSSSSESGVTTTIAILCHEIPHEMGDFAVLLSSGLSMKTAILMNFISSLT |
| | AFMGLYIGLSVSADPCVQDWIFTVTAGMFLYLSLVEMPEMTHVQTQRPWMMFLLQNFG |
| | LILGWLSLLLLAIYEQNIKI |

Further analysis of the NOV22a protean yielded the following properties shown in Table 22D.

TABLE 22B
Protein Sequence Properties NOV22a

| | |
|---|---|
| PSort | 0.6400 probability located in plasma membrane; 0.4600 |
| analysis: | probability located in Golgi body; 0.3700 probability |
| | located in endoplasmic reticulum (membrane); 0.1000 |
| | probability located in endoplasmic reticulum (lumen) |
| SignalP | Cleavage site between residues 24 and 25 |
| analysis: | |

A search of the NOV22a protein against the Geneseq database, a proprietary database that contains sequences published in patents and patent publication, yielded several homologous proteins shown in Table 22C.

TABLE 22C
Geneseq Results for NOV22a

| | | NOV22a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAB42004 | Human ORFX ORF1768 polypeptide | 81 . . . 248 | 163/168 (97%) | 5e−87 |
| | sequence SEQ ID NO: 3536-Homo | 1 . . . 163 | 163/168 (97%) | |
| | sapiens, 163 aa. [WO200058473-A2, | | | |
| | 5 Oct. 2000] | | | |
| ABB14720 | Human nervous system related | 1 . . . 167 | 160/167 (95%) | 9e−87 |
| | polypeptide SEQ ID NO 3377-Homo | 38 . . . 199 | 160/167 (95%) | |
| | sapiens, 206 aa. [WO200159063-A2, | | | |
| | 16 Aug. 2001] | | | |
| AAU74620 | Oestrogen-regulated LIV-1 family | 190 . . . 656 | 183/526 (34%) | 3e−76 |
| | protein BAB24106_Mm-Mus | 140 . . . 658 | 279/526 (52%) | |
| | musculus, 660 aa. [WO200196372-A2, | | | |
| | 20 Dec. 2001] | | | |
| AAU69470 | Human purified secretory polypeptide | 331 . . . 465 | 110/136 (80%) | 8e−54 |
| | #39-Homo sapiens, 172 aa. | 1 . . . 136 | 117/136 (85%) | |
| | [WO200162918-A2, 30 Aug. 2001] | | | |
| AAB59035 | Breast and ovarian cancer associated | 481 . . . 656 | 88/177 (49%) | 4e−44 |
| | antigen protein sequence SEQ ID 743- | 26 . . . 202 | 122/177 (68%) | |
| | Homo sapiens, 204 aa. [WO200055173- | | | |
| | A1, 21 Sep. 2000] | | | |

In a BLAST search of public sequence databases, the NOV22a protein was found to have homology to the proteins shown in the BLASTP data in Table 22D.

TABLE 22D
Public BLASTP Results for NOV22a

| | | NOV22a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q96NN4 | CDNA FLJ30499 fis, clone | 1 . . . 658 | 651/659 (98%) | 0.0 |
| | BRAWH2000443, weakly similar to human | 1 . . . 654 | 652/659 (98%) | |
| | breast cancer, estrogen regulated LIV-1 | | | |
| | protein (LIV-1) mRNA-Homo sapiens | | | |
| | (Human), 654 aa. | | | |
| Q95KA5 | Hypothetical 72.8 kDa protein-Macaca | 1 . . . 657 | 629/658 (95%) | 0.0 |
| | fascicularis (Crab eating macaque) | 1 . . . 653 | 642/658 (96%) | |
| | (Cynomolgus monkey), 654 aa. | | | |
| Q96LF0 | BA570F3.1 (Novel protein (Possible | 187 . . . 554 | 367/368 (99%) | 0.0 |
| | ortholog of a hypothetical protein from | 1 . . . 368 | 368/368 (99%) | |
| | macaca fascicularis clone QmoA-11613) | | | |
| | similar to hypothetical proteins from other | | | |
| | model organisms.)-Homo sapiens | | | |
| | (Human), 368 aa (fragment). | | | |
| Q9DAT9 | 1600025H15Rik protein (RIKEN cDNA | 190 . . . 656 | 183/526 (34%) | 8e−76 |
| | 1600025H15 gene)-Mus musculus | 140 . . . 658 | 279/526 (52%) | |
| | (Mouse), 660 aa. | | | |
| Q9H6T8 | CDNA: FLJ21884 fis, clone HEP02863- | 39 . . . 656 | 199/664 (29%) | 4e−71 |
| | Homo sapiens (Human), 647 aa. | 21 . . . 645 | 310/664 (45%) | |

PFam analysis predicts that the NOV22a protein contains the domains shown in the Table 22E.

TABLE 22E
Domain Analysis of NOV22a

| | | Identities/ | |
|---|---|---|---|
| Pfam | | Similarities for | Expect |
| Domain | NOV22a Match Region | the Matched Region | Value |
| Zip | 504 . . . 650 | 59/178 (33%) | 4e−34 |
| | | 117/178 (66%) | |

### Example 23

The NOV23 clone was analyzed, and the nucleotide and encoded polypeptide sequences are shown in Table 23A.

TABLE 23A
NOV23 Sequence Analysis

| | |
|---|---|
| SEQ ID NO: 67 | 1152 bp |

| | |
|---|---|
| NOV23a, | TACTGCCGCAGCGGAGTTCAGAGGGCCCGGAGGTGGGAGACTTCCCACACGGTGACTG |
| CG126481- | |
| 01 DNA | AGATGTCGTCCACTGCGGCTTTTTACCTTCTCTCTACGCTAGGAGGATACTTGGTGAC |
| Sequence | |
| | CTCATTCTTGTTGCTTAAATACCCGACCTTGCTGCACCAGAGAAAGAAGCAGCGATTC |
| | CTCAGTAAACACATCTCTCACCGCGGAGGTGCTGGAGAAAATTTGGAGAATACAATGG |
| | CAGCCTTTCAGCATGCGGTTAAAATCGGAACTGATATGCTAGAATTGGACTGCCATAT |
| | CACAAAAGATGAACAAGTTGTAGCGTCACATGATGAGAATCTAAAGAGAGCAACTGGG |
| | GTCAATGTAAACATCTCTGATCTCAAATACTGTGAGCTCCCACCTTACCTTGGCAAAC |
| | TGGATGTCTCATTTCAAAGAGCATGCCAGTGTGAAGGAAAAGATAACCGAATTCCATT |
| | ACTGAAGGAAGTTTTTGAGGCCTTTCCTAACACTCCCATTAACATCGATATCAAAGTC |
| | AACAACAATGTGCTGATTAAGAAGGTATCAGAGTTGGTGAAGCGGTATAATCGAGAAC |
| | ACTTAACAGTGTGGGGTAATGCCAATTATGAAATTGTAGAAAAGTGCTACAAAGAGAA |
| | TTCAGATATTCCTATACTCTTCAGTCTACAACGTGTCCTGCTCATTCTTGGCCTTTTC |
| | TTCACTGGCCTCTTGCCCTTTGTGCCCATTCGAGAACAGTTTTTTGAAATCCCAATGC |
| | CTTCTATTATACTGAAGCTAAAAGAACCACACACCATGTCCAGAAGTCAAAAGTTTCT |
| | CATCTGGCTTTCTGATCTCTTACTAATGAGGAAAGCTTTGTTTGACCACCTAACTGCT |
| | CGAGGCATTCAAGTGTATATTTGGGTATTAAATGAAGAACAAGAATACAAAAGAGCTT |
| | TTGATTTGGGAGCAACTGGGGTGATGACAGACTATCCAACAAAGCTTAGGGATTTTTT |
| | ACATAACTTTTCAGCATAGAAAAAGAGGTACTTAGAAGTATTGAAGGAAAAAATGAAG |
| | ACCTAAGAAAAAAATATTTCATGATCATTTCCCTAAGCCATTTCCAGAATGGTAAAAG |
| | GTTTAATCAGTTTTTATTACCTCATTTTTAAGCCTGTATGAGAATGTAGA |

| | |
|---|---|
| ORF Start: ATG at 61 | ORF Stop: EAG at 1003 |

| | | |
|---|---|---|
| SEQ ID NO:68 | 314 aa | MW at 36138.7 kD |

| | |
|---|---|
| NOV23a, | MSSTAAFYLLSTLGGYLVTSFLLLKYPTLLHQRKKQRFLSKHISHRGGAGENLENTMA |
| CG126481- | |
| 01 Protein | AFQHAVKIGTDMLELDCHITKDEQVVASHDENLKRATGVNVNISDLKYCBLPPYLGKL |
| Sequence | |
| | DVSFQRACQCEGKDNRIPLLKEVFEAFPNTPTNIDIKVNNNVLIKKVSELVKRYNREH |
| | LTVWGNANYEIVEKCYKENSDIPILFSLQRVLLILGLFFTGLLPFVPIREQFFETPMP |
| | SIILKLKEPHTMSRSQKFLIWLSDLLLMRKALFDHLTARGIQVYIWVLNEEQEYKRAF |
| | DLGATGVMTDYPTKLRDFLHNFSA |

| | |
|---|---|
| SEQ ID NO:69 | 1070 bp |

| | |
|---|---|
| NOV23b, | AGTTCAGAGGGCCCGGAGGTGGGAGACTTCCCACACGGTGACTGAGATGTCGTCCACT |
| CG126481- | |
| 02 DNA | GCGGCTTTTTACCTTCTCTCTACGCTAGGAGGATACTTGGTGACCTCATTCTTGTTGC |
| Sequence | |
| | TTAAATACCCGACCTTGCTGCACCAGAGAAAGAAGCAGCGATTCCTCAGTAAACACAT |
| | CTCTCACCGCGGAGGTGCTGGAGAAAATTTGGAGAATACAATGGCAGCCTTTCAGCAT |
| | GCGGTTAAAATCGGAACTGATATGCTAGAATTGGACTGCCATATCACAAAAGATGAAC |
| | AAGTTGTAGTGTCACATGATGAGAATCTAAAGAGAGCAACTGGGGTCAATGTAAACAT |
| | CTCTGATCTCAAATACTGTGAGCTCCCACCTTACCTTGGCAAACTGGATGTCTCATTT |
| | CAAAGAGCATGCCAGTGTGAAGGAAAAGATAACCGAATTCCATTACTGAAGGAAGTTT |
| | TTGAGGCCTTTCCTAACACTCCCATTAACATCGATATCAAAGTCAACAACAATGTGCT |
| | GATTAAGAAGGTTTCAGAGTTGGTGAAGCGGTATAATCGAGAACACTTAACAGTGTGG |
| | GGTAATGCCAATTATGAAATTGTAGAAAAGTGCTACAAAGAGAATTCAGATATTCCTA |
| | TACTCTTCAGTCTACAACGTGTCCTGCTCATTCTTGGCCTTTTCTTCACTGGCCTCTT |
| | GCCCTTTGTGCCCATTCGAGAACAGTTTTTTGAAATCCCAATGCCTTCTATTATACTG |
| | AAGCTAAAAGAACCACACACCATGTCCAGAAGTCAAAAGTTTCTCATCTGGCTTTCTG |
| | ATCTCTTACTAATGAGGAAAGCTTTGTTTGACCACCTAACTGCTCGAGGCATTCAAGT |
| | GTATATTTGGGTATTAAATGAAGAACAAGAATACAAAAGAGCTTTTGATTTGGGAGCA |
| | ACTGGGGTGATGACAGACTATCCAACAAAGCTTAGGGATTTTTTACATAACTTTTCAG |
| | CATAGAAAAAGAGGTACTTAGAAGTATTGAATTAAAAAATGAAGACCTAAGAAAAAAA |
| | TATTTCATGATCATTTCCCTAAGCCA |

| | |
|---|---|
| ORF Start: ATG at 47 | ORF Stop: TAG at 989 |

| | | |
|---|---|---|
| SEQ ID NO:70 | 314 aa | MW at 36166.7 kD |

| | |
|---|---|
| NOV23b, | MSSTAAFYLLSTLGGYLVTSFLLLKYPTLLHQRKKQRFLSKHTSHRGGAGENLENTMA |
| CG126481- | |
| 02 Protein | AFQHAVKIGTDMLELDCHITKDEQVVVSHDENLKRATGVNVNISDLKYCELPPYLGKL |
| Sequence | |
| | DVSFQRACQCEGKDNRIPLLKEVFEAFPNTPINIDIKVNNNVLIKKVSELVKRYNREH |
| | LTVWGNANYEIVEKCYKENSDIPILFSLQRVLLILGLFFTGLLPFVPIREQFFEIPMP |
| | SIILKLKEPHTMSRSQKFLIWLSDLLLMRKALFDHLTARGIQVYIWVLNEEQEYKRAF |
| | DLGATGVMTDYPTKLRDFLHNFSA |

| | |
|---|---|
| SEQ ID NO:71 | 961 bp |

| | |
|---|---|
| NOV23c, | CACCGGATCCATGTCGTCCACTGCGGCTTTTTACCTTCTCTCTACGCTAGGAGGATAC |
| 278459554 | |
| DNA | TTGGTGACCTCATTCTTGTTGCTTAAATACCCGACCTTGCTGCACCAGAGAAAGAAGC |
| Sequence | |
| | AGCGATTCCTCAGTAAACACATCTCTCACCGCGGAGGTGCTGGAGAAAATTTGGAGAA |
| | TACAATGGCAGCCTTTCAGCATGCGGTTAAAATCGGAACTGATATGCTAGAATTGGAC |
| | TGCCATATCACAAAAGATGAACAAGTTGTAGTGTCACATGATGAGAATCTAAAGAGAG |
| | CAACTGGGGTCAATGTAAACATCTCTGATCTCAAATACTGTGAGCTCCCACCTTACCT |
| | TGGCAAACTGGATGTCTCATTTCAAAGAGCATGCCAGTGTGAAGGAAAAGATAACCGA |
| | ATTCCATTACTGAAGGAAGTTTTTGAGGCCTTTCCTAACACTCCCATTAACATCGATA |
| | TCAAAGTCAACAACAATGTGCTGATTAAGAAGGTTTCAGAGTTGGTGAAGCGGTATAA |
| | TCGAGAACACTTAACAGTGTGGGGTAATGCCAATTATGAAATTGTAGAAAAGTGCTAC |
| | AAAGAGAATTCAGATATTCCTATACTCTTCAGTCTACAACGTGTCCTGCTCATTCTTG |
| | GCCTTTTCTTCACTGGCCTCTTGCCCTTTGTGCCCATTCGAGAACAGTTTTTTGAAAT |
| | CCCAATGCCTTCTATTATACTGAAGCTAAAAGAACCACACACCATGTCCAGAAGTCAA |
| | AAGTTTCTCATCTGGCTTTCTGATCTCTTACTAATGAGGAAAGCTTTGTTTGACCACC |
| | TAACTGCTCGAGGCATTCAAGTGTATATTTGGGTATTAAATGAAGAACAAGAATACAA |
| | AAGAGCTTTTGATTTGGGAGCAACTGGGGTGATGACAGACTATCCAACAAAGCTTAGG |
| | GATTTTTTACATAACTTTTCAGCAGTCGACGGC |

| | |
|---|---|
| ORF Start: at 2 | ORF Stop: end of sequence |

| | | |
|---|---|---|
| SEQ ID NO: 72 | 320 aa | MW at 36683.2 kD |

| | |
|---|---|
| NOV23c, | TGSMSSTAAFYLLSTLGGYLVTSFLLLKYPTLLHQRKKQRFLSKHISHRGGAGENLEN |
| 278459554 | |
| Protein | TMAAFQHAVKIGTDMLELDCHTTKDEQVVVSHDENLKRATGVNVNISDLKYCELPPYL |
| Sequence | |
| | GKLDVSFQRACQCEGKDNRIPLLKEVFEAFPNTPINIDIKVNNNVLIKKVSELVKRYN |
| | REHLTVWGNANYEIVEKCYKENSDIPILFSLQRVLLILGLFFTGLLPFVPIREQFFEI |
| | PMPSIILKLKEPHTMSRSQKFLIWLSDLLLMRKALFDHLTARGIQVYIWVLNEEQEYK |
| | RAFDLGATGVMTDYPTKLRDFLHNFSAVDG |

| | |
|---|---|
| SEQ ID NO:73 | 865 bp |

| | |
|---|---|
| NOV23d, | CACCGGATCCAGAAAGAAGCAGCGATTCCTCAGTAAACACATCTCTCACCGCGGAGGT |
| 278463211 | |
| DNA | GCTGGAGAAAATTTGGAGAATACAATGGCAGCCTTTCAGCATGCGGTTAAAATCGGAA |
| Sequence | |
| | CTGATATGCTAGAATTGGACTGCCATATCACAAAAGATGAACAAGTTGTAGTGTCACA |
| | TGATGAGAATCTAAAGAGAGCAACTGGGGTCAATGTAAACATCTCTGATCTCAAATAC |
| | TGTGAGCTCCCACCTTACCTTGGCAAACTGGATGTCTCATTTCAAAGAGCATGCCAGT |
| | GTGAAGGAAAAGATAACCGAATTCCATTACTGAAGGAAGTTTTTGAGGCCTTTCCTAA |
| | CACTCCCATTAACATCGATATCAAAGTCAACAACAATGTGCTGATTAAGAAGGTTTCA |
| | GAGTTGGTGAAGCGGTATAATCGAGAACACTTAACAGTGTGGGGTAATGCCAATTATG |
| | AAATTGTAGAAAAGTGCTACAAAGAGAATTCAGATATTCCTATACTCTTCAGTCTACA |
| | ACGTGTCCTGCTCATTCTTGGCCTTTTCTTCACTGGCCTCTTGCCCTTTGTGCCCATT |
| | CGAGAACAGTTTTTTGAAATCCCAATGCCTTCTATTATACTGAAGCTAAAAGAACCAC |
| | ACACCATGTCCAGAAGTCAAAAGTTTCTCATCTGGCTTTCTGATCTCTTACTAATGAG |
| | GAAAGCTTTGTTTGACCACCTAACTGCTCGAGGCATTCAAGTGTATATTTGGGTATTA |
| | AATGAAGAACAAGAATACAAAAGAGCTTTTGATTTGGGAGCAACTGGGGTGATGACAG |
| | ACTATCCAACAAAGCTTAGGGATTTTTTACATAACTTTTCAGCAGTCGACGGC |

| | |
|---|---|
| ORF Start: at 2 | ORF Stop: end of sequence |

| | | |
|---|---|---|
| SEQ ID NO: 74 | 288 aa | MW at 33151.1 kD |

| | |
|---|---|
| NOV23d, | TGSRKKQRFLSKHISHRGGAGENLENTMAAFQHAVKIGTDMLELDCHITKDEQVVVSH |
| 278463211 | |
| Protein | DENLKRATGVNVNISDLKYCELPPYLGKLDVSFQRACQCEGKDNRIPLLKEVFEAFPN |
| Sequence | |
| | TPINIDIKVNNNVLIKKVSELVKRYNREHLTVWGNANYEIVEKCYKENSDIPILFSLQ |
| | RVLLILGLFFTGLLPFVPIREQFFEIPMPSIILKLKEPHTMSRSQKFLIWLSDLLLMR |
| | KALFDHLTARGIQVYIWVLNEEQEYKRAFDLGATGVMTDYPTKLRDFLHNFSAVDG |

| | |
|---|---|
| SEQ ID NO: 75 | 805 bp |

| | |
|---|---|
| NOV93e, | CACCGGATCCCACCGCGGAGGTGCTGGAGAAAATTTGGAGAATACAATGGCAGCCTTT |
| 278465805 | |
| DNA | CAGCATGCGGTTAAAATCGGAACTGATATGCTAGAATTGGACTGCCATATCACAAAAG |
| Sequence | |
| | ATGAACAAGTTGTAGTGTCACATGATGAGAATCTAAAGAGAGCAACTGGGGTCAATGT |
| | AAACATCTCTGATCTCAAATACTGTGAGCTCCCACCTTACCTTGGCAAACTGGATGTC |
| | TCATTTCAAAGAGCATGCCAGTGTGAAGGAAAAGATAACCGAATTCCATTACTGAAGG |
| | AAGTTTTTGAGGCCTTTCCTAACACTCCCATTAACATCGATATCAAAGTCAACAACAA |
| | TGTGCTGATTAAGAAGGTTTCAGAGTTGGTGAAGCGGTATAATCGAGAACACTTAACA |
| | GTGTGGGGTAATGCCAATTATGAAATTGTAGAAAAGTGCTACAAAGAGAATTCAGATA |
| | TTCCTATACTCTTCAGTCTACAACGTGTCCTGCTCATTCTTGGCCTTTTCTTCACTGG |
| | CCTCTTGCCCTTTGTGCCCATTCGAGAACAGTTTTTTGAAATCCCAATGCCTTCTATT |
| | ATACTGAAGCTAAAAGAACCACACACCATGTCCAGAAGTCAAAAGTTTCTCATCTGGC |
| | TTTCTGATCTCTTACTAATGAGGAAAGCTTTGTTTGACCACCTAACTGCTCGAGGCAT |
| | TCAAGTGTATATTTGGGTATTAAATGAAGAACAAGAATACAAAAGAGCTTTTGATTTG |
| | GGAGCAACTGGGGTGATGACAGACTATCCAACAAAGCTTAGGGTCGACGGC |

| | |
|---|---|
| ORF Start: at 2 | ORF Stop: end of sequence |

| | | |
|---|---|---|
| SEQ ID NO: 76 | 268 aa | MW at 30709.3 kD |

| | |
|---|---|
| NOV23e, | TGSHRGGAGENLENTMAAFQHAVKIGTDMLELDCHITKDEQVVVSHDENLKRATGVNV |
| 278465805 | |
| Protein | NISDLKYCELRPYLGKLDVSFQRACQCEGKDNRIPLLKEVFEAFPNTPINIDIKVNNN |
| Sequence | |
| | VLIKKVSELVKRYNREHLTVWGNANYEIVEKCYKENSDIPILFSLQRVLLILGLFFTG |
| | LLPFVPIREQFFETPMPSIILKLKEPHTMSRSQKFLIWLSDLLLMRKALFDHLTARGI |
| | QVYIWVLNEEQEYKRAFDLGATGVMTDYPTKLRVDG |

Sequence comparison of the above protein sequences yields the following sequence relationships shown in Table 23B.

TABLE 23B
Comparison of NOV23a against NOV23b through NOV23e

| | | Identities/ |
|---|---|---|
| | NOV23a Residues/ | Similarities for |
| Protein Sequence | Match Residues | the Matched Region |
| NOV23b | 1 . . . 314 | 301/314 (95%) |
| | 1 . . . 314 | 301/314 (95%) |
| NOV23c | 1 . . . 314 | 301/314 (95%) |
| | 4 . . . 317 | 301/314 (95%) |
| NOV23d | 33 . . . 314 | 269/282 (95%) |
| | 4 . . . 285 | 269/282 (95%) |
| NOV23e | 44 . . . 306 | 250/263 (95%) |
| | 3 . . . 265 | 250/263 (95%) |

Further analysis of the NOV23a protein yielded the following properties shown in Table 23C.

TABLE 23C
Protein Sequence Properties NOV23a

| | |
|---|---|
| PSort | 0.7300 probability located in plasma membrane; 0.6400 |
| analysis: | probability located in endoplasmic reticulum (membrane); |
| | 0.1000 probability located in endoplasmic reticulum |
| | (lumen); 0.1000 probability located in outside |
| SignalP | Cleavage site between residues 33 and 34 |
| analysis: | |

search of the NOV23a protein against the Geneseq database, a proprietary database that contains sequences published in patents and patent publications yielded several homologous proteins shown in Table 23D.

TABLE 23D
Geneseq Results for NOV23a

| | | NOV23a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAM49156 | Human Myb protein 32-Homo | 1 . . . 268 | 265/268 (98%) | e−152 |
| | sapiens, 289 aa. [CN1325886-A, | 1 . . . 268 | 266/268 (98%) | |
| | 12 Dec. 2001] | | | |
| ABB09007 | Human phosphodiesterase-3-Homo | 1 . . . 192 | 191/192 (99%) | e−109 |
| | sapiens, 210 aa. [WO200198471-A2, | 1 . . . 192 | 191/192 (99%) | |
| | 27 Dec. 2001] | | | |
| AAE05493 | Human phosphodiesterase-3 (HPDE- | 3 . . . 311 | 129/309 (41%) | 5e−70 |
| | 3)-Homo sapiens, 318 aa. | 2 . . . 310 | 198/309 (63%) | |
| | [WO200155358-A2, 2 Aug. 2001] | | | |
| AAU27639 | Human protein AFP471025-Homo | 3 . . . 303 | 125/301 (41%) | 1e−67 |
| | sapiens, 330 aa. [WO200166748-A2, | 2 . . . 302 | 192/301 (63%) | |
| | 13 Sep. 2001] | | | |
| AAM41071 | Human polypeptide SEQ ID NO 6002- | 68 . . . 311 | 104/244 (42%) | 9e−55 |
| | Homo sapiens, 300 aa. | 50 . . . 292 | 159/244 (64%) | |
| | [WO200153312-A1, 26 Jul. 2001] | | | |

In a BLAST search of public sequence databases, the NOV23a protein was found to have homology to the proteins shown in the BLASTP data in Table 23E.

TABLE 23E
Public BLASTP Results for NOV23a

| | | NOV23a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9CRY7 | 2610020H15Rik protein (RIKEN | 1 . . . 314 | 288/314 (91%) | e−168 |
| | cDNA 2610020H15 gene)-Mus | 1 . . . 314 | 299/314 (94%) | |
| | musculus (Mouse), 314 aa (fragment). | | | |
| Q9D4X7 | 2610020H15Rik protein-Mus | 1 . . . 314 | 287/314 (91%) | e−167 |
| | musculus (Mouse), 314 aa. | 1 . . . 314 | 298/314 (94%) | |
| Q9CT14 | 2610020H15Rik protein-Mus | 51 . . . 314 | 236/264 (89%) | e−137 |
| | musculus (Mouse), 341 aa (fragment). | 78 . . . 341 | 247/264 (93%) | |
| CAC88621 | Sequence 51 from Patent WO0166748- | 3 . . . 303 | 125/301 (41%) | 3e−67 |
| | Homo sapiens (Human), 330 aa. | 2 . . . 302 | 192/301 (63%) | |
| Q9D1C0 | 1110015E22Rik protein-Mus | 7 . . . 309 | 121/303 (39%) | 1e−64 |
| | musculus (Mouse), 330 aa. | 6 . . . 308 | 188/303 (61%) | |

PFam analysis predicts that the NOV23a protein contains the domains shown in the Table 23F.

TABLE 23F
Domain Analysis of NOV23a

| | | Identities/ | |
|---|---|---|---|
| Pfam | | Similarities for | Expect |
| Domain | NOV23a Match Region | the Matched Region | Value |
| GDPD | 45 . . . 306 | 60/283 (21%) | 3.6e−19 |
| | | 179/283 (63%) | |

### Example 24

The NOV24 clone was analyzed, and the nucleotide and encoded polypeptide sequences are shown in Table 24A.

TABLE 24A
NOV24 Sequence Analysis

| | |
|---|---|
| SEQ ID NO: 77 | 1092 bp |

| | |
|---|---|
| NOV24a, | CTTAGCGAGCGCTGGAGTTTGAAGAGCGGGCAGTGGCTGCACACGCCAAACTTTCCCT |
| CG127851- | |
| 01 DNA | ATGGCTTCGGTGACCAGGGCCGTGTTTGGAGAGCTGCCCTCGGGAGGAGGGACAGTGG |
| Sequence | |
| | AGAAGTTCCAGCTGCAGTCAGACCTCTTGAGAGTGGACATCATCTCCTGGGGCTGCAC |
| | GATCACAGCCCTAGAGGTCAAAGACAGGCAGGGGAGAGCCTCGGACGTGGTGCTTGGC |
| | TTCGCCGAGTTGGAAGTGTACCTCCAAAAGCAGCCATACTTTGGAGCAGTTATTGGGA |
| | GGGTGGCCAACCGAATCGCCAAAGGAACCTTCAAGGTGGATGGGAAGGAGTATCACCT |
| | GGCCATTAACAAGGAACCCAACAGTCTGCATGGAGGAGTCAGAGGGTTTGATAAAGTA |
| | CTATGGACCCCTCGGGTGCTGTCAAATGGCGTCCAGTTCTCGCGCATCAGTCCAGATG |
| | GTGAAGAAGGCTACCCCGGAGAGTTAAAAGTCTGGGTGACATACACCCTGGATGGCGG |
| | AGAGCTCATAGTCAACTACAGAGCACAAGCCAGTCAGGCCACACCAGTCAACCTGACC |
| | AACCATTCTTACTTCAACCTGGCAGGCCAGGCTTCCCCAAATATAAATGACCATGAAG |
| | TCACCATAGAAGCGGATACTTATTTGCCTGTGGATGAAACCCTGATTCCTACAGGTGA |
| | GGTTGCCCCAGTGCAAGGCACTGCATTCGACCTGAGAAAGCCAGTGGAGCTTGGAAAA |
| | CACCTGCAGGACTTCCATCTCAATGGTTTTGACCACAATTTCTGTCTGAAGGGATCTA |
| | AAGAAAAGCATTTTTGTGCAAGGGTGCATCATGCTGCAAGCGGGCGGGTACTAGAAGT |
| | ATACACCACCCAGCCCGGGGTCCAGTTTTACACGGGCAACTTCCTGGATGGCACATTA |
| | AAGGGCAAGAATGGAGCTGTCTATCCCAAGCACTCCGGTTTCTGCCTGGAGACTCAGA |
| | ACTGGCCTGATGCAGTCAATCAGCCCCGCTTCCCTCCTGTGCTGCTGAGGCCTGGTGA |
| | GGAGTATGACCACACCACCTGGTTCAAGTTTTCTGTGGCTTAAGGAAG |

| | |
|---|---|
| ORF Start: ATG at 59 | ORF Stop: TAA at 1085 |

| | | |
|---|---|---|
| SEQ ID NO: 78 | 342 aa | MW at 37807.4 kD |

| | |
|---|---|
| NOV24a, | MASVTRAVFGELRSGGGTVEKPQLQSDLLRVDIISWGCTITALEVKDRQGRASDVVLG |
| CG127851- | |
| 01 Protein | FAELEVYLQKQRYFGAVIGRVANRIAKGTFKVDGKEYHLAINKEPNSLHGGVRGFDKV |
| Sequence | |
| | LWTPRVLSNGVQFSRISPDGEEGYPGELKVWVTYTLDGGELIVNYRAQASQATPVNLT |
| | NHSYFNLAGQASPNINDHEVTIEADTYLPVDETLIPTGEVAPVQGTAFDLRKPVELGK |
| | HLQDFHLNGFDHNFCLKGSKEKHFCARVHHAASGRVLEVYTTQPGVQFYTGNFLDGTL |
| | KGKNGAVYPKHSGFCLETQNWPDAVNQPRFPPVLLRPGEEYDHTTWFKFSVA |

| | |
|---|---|
| SEQ ID NO: 79 | 1099 bp |

| | |
|---|---|
| NOV24b, | CGCCCTTCTTAGCGAGCGCTGGAGTTTGAAGAGCGGGCAGTGGCTGCACACGCCAAAC |
| CG127851- | |
| 02 DNA | TTTCCCTATGGCTTCGGTGACCAGGGCCGTGTTTGGAGAGCTGCCCTCGGGAGGAGGG |
| Sequence | |
| | ACAGTGGAGAAGTTCCAGCTGCAGTCAGACCTCTTGAGAGTGGACATCATCTCCTGGG |
| | GCTGCACGATCACAGCCCTAGAGGTCAAAGACAGGCAGGGGAGAGCCTCGGACGTGGT |
| | GCTTGGCTTCGCCGAGTTGGAAGGATACCTCCAAAAGCAGCCATACTTTGGAGCAGTT |
| | ATTGGGAGGGTGGCCAACCGAATCGCCAAAGGAACCTTCAAGGTGGATGGGAAGGAGT |
| | ATCACCTGGCCATTAACAAGGAACCCAACAGTCTGCATGGAGGAGTCAGAGGGTTTGA |
| | TAAAGTGCTCTGGACCCCTCGGGTGCTGTCAAATGGCGTCCAGTTCTCGCGCATCAGT |
| | CCAGATGGTGAAGAAGGCTACCCCGGAGAGTTAAAAGTCTGGGTGACATACACCCTGG |
| | ATGGCGGAGAGCTCATAGTCAACTACAGAGCACAAGCCAGTCAGGCCACACCAGTCAA |
| | CCTGACCAACCATTCTTACTTCAACCTGGCAGGCCAGGCTTCCCCAAATATAAATGAC |
| | CATGAAGTCACCATAGAAGCGGATACTTATTTGCCTGTGGATGAAACCCTGATTCCTA |
| | CAGGAGAAGTTGCCCCAGTGCAAGGCACTGCATTCGACCTGACAAAGCCAGTGGAGCT |
| | TGGAAAACACCTGCAGGACTTCCATCTCAATGGTTTTGACCACAATTTCTGTCTGAAG |
| | GGATCTAAAGAAAAGCATTTTTGTGCAAGGGTGCATCATGCTGCAAGCGGGCGGGTAC |
| | TAGAAGTATACACCACCCAGCCCGGGGTCCAGTTTTACACGGGCAACTTCCTGGATGG |
| | CACATTAAAGGGCAAGAATGGAGCTGTCTATCCCAAGCACTCCGGTTTCTGCCTGGAG |
| | ACTCAGAACTGGCCTGATGCAGTCAATCAGCCCCGCTTCCCTCCTGTGCTGCTGAGGC |
| | CTGGTGAGGAGTATGACCACACCACCTGGTTCAAGTTTTCTGTGGCTTAAGGAAG |

| | |
|---|---|
| ORF Start: ATG at 66 | ORF Stop: TAA at 1092 |

| | | |
|---|---|---|
| SEQ ID NO: 80 | 1342 aa | MW at 37710.2 kD |

| | |
|---|---|
| NOV24b, | MASVTRAVFGELPSGGGTVEKFQLQSDLLRVDIISWGCTITALEVKDRQGRASDVVLG |
| CG127851- | |
| 02 Protein | FAELEGYLQKQPYFGAVIGRVANRIAKGTFKVDGKEYHLAINKEPNSLHGGVRGFDKV |
| Sequence | |
| | LWTPRVLSNGVQFSRISPDGEEGYPGELKVWVTYTLDGGELIVNYRAQASQATPVNLT |
| | NHSYFNLAGQASPNINDHEVTIEADTYLPVDETLIPTGEVAPVQGTAFDLTKPVELGK |
| | HLQDFHLNGFDHNFCLKGSKEKHFCARVHHAASGRVLEVYTTQPGVQFYTGNFLDGTL |
| | KGKNGAVYPKHSGFCLETQNWPDAVNQPRFPPVLLRPGEEYDHTTWFKFSVA |

Sequence comparison of the above protein sequences yields the following sequence relationships shown in Table 24B.

TABLE 24B
Comparison of NOV24a against NOV24b.

| | | Identities/ |
|---|---|---|
| | NOV24a Residues/ | Similarities for |
| Protein Sequence | Match Residues | the Matched Region |
| NOV24b | 1 . . . 342 | 340/342 (99%) |
| | 1 . . . 342 | 340/342 (99%) |

Further analysis of the NOV24a protein yielded the following properties shown in Table 24C.

TABLE 24C
Protein Scquence Properties NOV24a

| | |
|---|---|
| PSort | 0.6400 probability located in microbody (peroxisome): 0.4500 |
| analysis: | probability located in cytoplasm; 0.2445 probability located |
| | in lysosome (lumen); 0.1000 probability located in |
| | mitochondrial matrix space |
| SignalP | No Known Signal Sequence Predicted |
| analysis: | |

A search of the NOV24a protein against the Geneseq database, a proprietary database that contains sequences published in patents and patent publication, yielded several homologous proteins shown in Table 24D.

TABLE 24D
Geneseq Results for NOV24a

| | | NOV24a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAR70142 | Porcine mutarotase (MUT) enzyme- | 3 . . . 342 | 305/340 (89%) | 0.0 |
| | Sus scrofa, 341 aa. [JP07039380-A, | 2 . . . 341 | 322/340 (94%) | |
| | 10 Feb. 1995] | | | |
| AAR72964 | Pig kidney cell mutarotase protein-Sus | 3 . . . 342 | 305/340 (89%) | 0.0 |
| | scrofa, 341 aa. [JP06253856-A, | 2 . . . 341 | 322/340 (94%) | |
| | 13 Sep. 1994] | | | |
| AAM40101 | Human polypeptide SEQ ID NO 3246- | 1 . . . 259 | 258/259 (99%) | e−150 |
| | Homo sapiens, 268 aa. [WO200153312- | 1 . . . 259 | 258/259 (99%) | |
| | A1, 26 Jul. 2001] | | | |
| AAG49126 | Arabidopsis thaliana protein fragment | 18 . . . 340 | 153/336 (45%) | 2e−76 |
| | SEQ ID NO: 62115-Arabidopsis | 8 . . . 340 | 215/336 (63%) | |
| | thaliana, 341 aa. [EP1033405-A2, | | | |
| | 6 Sep. 2000] | | | |
| AAG49127 | Arabidopsis thaliana protein fragment | 29 . . . 340 | 149/325 (45%) | 2e−74 |
| | SEQ ID NO: 62116-Arabidopsis | 1 . . . 322 | 209/325 (63%) | |
| | thaliana, 323 aa. [EP1033405-A2, | | | |
| | 6 Sep. 2000] | | | |

In a BLAST search of public sequence databases, the NOV24a protein was found to have homology to the proteins shown in the BLASTP data in Table 24E.

TABLE 24E
Public BLASTP Results for NOV24a

| | | NOV22a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q96C23 | Hypothetical 37.8 kDa protein- | 1 . . . 342 | 341/342 (99%) | 0.0 |
| | Homo sapiens (Human), 342 aa. | 1 . . . 342 | 341/342 (99%) | |
| Q9GKX6 | Aldose 1-epimerase (EC 3.1.3.3)- | 1 . . . 342 | 306/342 (89%) | 0.0 |
| | Sus scrofa (Pig), 342 aa. | 1 . . . 342 | 323/342 (93%) | |
| AAH28818 | Similar to hypothetical protein | 1 . . . 342 | 297/342 (86%) | 0.0 |
| | BC014916-Mus musculus | 1 . . . 342 | 318/342 (92%) | |
| | (Mouse), 342 aa. | | | |
| AAL62475 | BLOCK 25-Homo sapiens | 1 . . . 212 | 211/212 (99%) | e−120 |
| | (Human), 221 aa. | 1 . . . 212 | 211/212 (99%) | |
| Q9RDN0 | Putative aldose 1-epimerase- | 6 . . . 339 | 159/346 (45%) | 8e−81 |
| | Streptomyces coelicolor, 366 aa. | 20 . . . 364 | 220/346 (62%) | |

PFam analysis predicts that the NOV24a protein contains the domains shown in the Table 24F.

TABLE 24F
Domain Analysis of NOV24a

| | | Identities/ | |
|---|---|---|---|
| Pfam | NOV24a | Similarities for | Expect |
| Domain | Match Region | the Matched Region | Value |
| Aldose_epim | 8 . . . 340 | 140/371 (38%) | 2.4e−104 |
| | | 243/371 (65%) | |

### Example 25

The NOV25 clone was analyzed, and the nucleotide and encoded polypeptide sequences are shown in Table 25A.

TABLE 25A
NOV25 Sequence Analysis

| | |
|---|---|
| SEQ ID NO:81 | 1197 bp |

| | |
|---|---|
| NOV25a. | TTGATACTTCCGCAAATGGGAAAATCTGGAGTCCTGGAAAGCCCCAGGAGCTCTGATA |
| CG 127906- | |
| 01 DNA | TTCTCTGCACACACGCAGAGGCAAGTAACACACACACTTTGTTGCTGCAGAATGACTC |
| Sequence | |
| | GCGTTGGAGCCTTTTGTGCCAGGAGGAGGGGACCTGGTTTCTGGCTGGAATCAGAGAC |
| | TTTCCCAGTGGCTGTCTACGTCCCCGAGCCTTCTTCCCTCTGCAGACTCATGGCCCAT |
| | GGATCAGCCATGTGACTCGGGGAGCCTACCTGGAGGACCAGCTAGCCTGGGATTGGGG |
| | CCCTGATGGGGAGGAGACTGAGACACAGACTTGTCCCCCACACACAGAGCATGGTGCC |
| | TGTGGCCTGCGGCTGGAGGCTGCTCCAGTGGGGGTCCTGTGGCCCTGGCTGGCAGAGG |
| | TGCATGTGGCTGGTGATCGAGTCTGCACTGGGATCCTCCTGGCCCCAGGCTGGGTCCT |
| | GGCAGCCACTCACTGTGTCCTCAGGCCAGGCTCTACAACAGTGCCTTACATTGAAGTG |
| | TATCTGGGCCGGGCAGGGGCCAGCTCCCTCCCACAGGGCCACCAGGTATCCCGCTTGG |
| | TCATCAGCATCCGGCTGCCCCAGCACCTGGGACTCAGGCCCCCCCTGGCCCTCCTGGA |
| | GCTGAGCTCCCGGGTGGAGCCCTCCCCATCAGCCCTGCCCATCTGTCTCCACCCGGCG |
| | GGTATCCCCCCGGGGGCCAGCTGCTGGGTGTTGGGCTGGAAAGAACCCCAGGACCGAG |
| | TCCCTGTGGCTGCTGCTGTCTCCATCTTGACACAACGAATCTGTGACTGCCTCTATCA |
| | GGGCATCCTGCCCCCTGGAACCCTCTGTGTCCTGTATGCAGAGGGGCAGGAGAACAGG |
| | TGTGAGATGACCTCAGCACCGCCCCTCCTGTGCCAGATGACGGAAGGGTCCTGGATCC |
| | TCGTGGGCATGGCTGTTCAAGGGAGCCGGGAGCTGTTTGCTGCCATTGGTCCTGAAGA |
| | GGCCTGGATCTCCCAGACAGTGGGAGAGGCCAACTTCCTGCCCCCCAGTGGCTCCCCA |
| | CACTGGCCCACTGGAGGCAGCAATCTCTGCCCCCCAGAACTGGCCAAGGCCTCGGGAT |
| | CCCCGCATGCAGTCTACTTCCTGCTCCTGCTGACTCTCCTGATCCAGAGCTGAGGGGC |
| | TAGGGTCCCAGCACCACTTCCCCCTTCTCCACCCTCT |

| | |
|---|---|
| ORF Start: ATG at 16 | ORF Stop: TGA at 1153 |

| | | |
|---|---|---|
| SEQ ID NO: 82 | 379 aa | MW at 40786.3 kD |

| | |
|---|---|
| NOV25a, | MGKSGVLESPRSSDILCTHAEASNTHTLLLQNDSRWSLLCQEEGTWFLAGIRDFPSGC |
| CG127906- | |
| 01 Protein | LRPRAFFPLQTHGPWISHVTRGAYLEDQLAWDWGPDGEETETQTCPPHTEHGACGLRL |
| Sequence | |
| | EAAPVGVLWPWLAEVHVAGDRVCTGILLAPGWVLAATHCVLRPGSTTVPYIEVYLGRA |
| | GASSLPQGHQVSRLVISIRLPQHLGLRPPLALLELSSRVEPSPSALPICLHPAGIPPG |
| | ASCWVLGWKEPQDRVPVAAAVSILTQRICDCLYQGILPPGTLCVLYAEGQENRCEMTS |
| | APPLLCQMTEGSWILVGMAVQGSRELFAAIGPEEAWISQTVGEANFLPPSGSPHWPTG |
| | GSNLCPPELAKASGSPHAVYFLLLLTLLIQS |

Further analysis of the NOV25a protein yielded the following properties shown in Table 25B.

TABLE 25B
Protein Sequence Properties NOV25a

| | |
|---|---|
| PSort | 0.4526 probability located in microbody (peroxisome); 0.4500 |
| analysis: | probability located in cytoplasm; 0.2266 probability located |
| | in lysosome (lumen); 0.1000 probability located in |
| | mitochondrial matrix space |
| SignalP | No Known Signal Sequence Predicted |
| analysis: | |

A search of the NOV25a protein against the Geneseq database, a proprietary database that contains sequences published in patents and patent publication, yielded several homologous proteins shown in Table 25C.

TABLE 25C
Geneseq Results for NOV25a

| | | NOV25a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAM93568 | Human polypeptide, SEQ ID NO: 3347- | 32 . . . 379 | 347/348 (99%) | 0.0 |
| | Homo sapiens, 766 aa. [EP1130094- | 419 . . . 766 | 347/348 (99%) | |
| | A2, 5 Sep. 2001] | | | |
| AAU82753 | Amino acid sequence of novel human | 69 . . . 379 | 311/311 (100%) | 0.0 |
| | protease #52-Homo sapiens, 818 aa. | 508 . . . 818 | 311/311 (100%) | |
| | [WO200200860-A2, 3 Jan. 2002] | | | |
| ABG06892 | Novel human diagnostic protein #6883- | 69 . . . 379 | 300/311 (96%) | 0.0 |
| | Homo sapiens, 692 aa. | 392 . . . 692 | 300/311 (96%) | |
| | [WO200175067-A2, 11 Oct. 2001] | | | |
| ABG06892 | Novel human diagnostic protein #6883- | 69 . . . 379 | 300/311 (96%) | 0.0 |
| | Homo sapiens, 692 aa. | 392 . . . 692 | 300/311 (96%) | |
| | [WO200175067-A2, 11 Oct. 2001] | | | |
| AAE06934 | Human membrane-type serine protease | 108 . . . 312 | 70/225 (31%) | 2e−22 |
| | (MTSP) 4-S splice variant-Homo | 411 . . . 631 | 109/225 (48%) | |
| | sapiens, 658 aa. [WO200157194-A2, | | | |
| | 9 Aug. 2001] | | | |

In a BLAST search of public sequence databases, the NOV25a protein was found to have homology to the proteins shown in the BLASTP data in Table 25D.

TABLE 25D
Public BLASTP Results for NOV25a

| | | NOV25a | | |
|---|---|---|---|---|
| Protein | | Residues/ | Identities/ | |
| Accession | | Match | Similarities for | Expect |
| Number | Protein/Organism/Length | Residues | the Matched Portion | Value |
| CAC60381 | Sequence 9 from Patent WO0157194- | 108 . . . 312 | 70/225 (31%) | 5e−22 |
| | Homo sapiens (Human), 658 aa. | 411 . . . 631 | 109/225 (48%) | |
| CAC60380 | Sequence 7 from Patent WO0157194- | 108 . . . 312 | 70/225 (31%) | 5e−22 |
| | Homo sapiens (Human), 802 aa. | 555 . . . 775 | 109/225 (48%) | |
| CAC60379 | Sequence 5 from Patent WO0157194- | 125 . . . 312 | 64/201 (31%) | 2e−21 |
| | Homo sapiens (Human), 235 aa | 12 . . . 208 | 98/201 (47%) | |
| | (fragment) | | | |
| Q9QUL7 | Tryptase gamma precursor (EC | 118 . . . 346 | 77/249 (30%) | 6e−21 |
| | 3.4.21.-) (Transmembrane tryptase)- | 35 . . . 276 | 107/249 (42%) | |
| | Mus musculus (Mouse), 311 aa. | | | |
| Q9DB10 | 1300008A22Rik protein-Mus | 108 . . . 312 | 70/225 (31%) | 2e−20 |
| | musculus (Mouse), 799 aa. | 552 . . . 772 | 105/225 (46%) | |

PFam analysis predicts that the NOV25a protein contains the domains shown in the Table 25E.

TABLE 25E
Domain Analysis of NOV25a

| | | Identities/ | |
|---|---|---|---|
| Pfam | | Similarities for | Expect |
| Domain | NOV25a Match Region | the Matched Region | Value |
| Trypsin | 125 . . . 240 | 41/140 (29%) | 2.6e−09 |
| | | 74/140 (53%) | |

### Example 26

The NOV26 clone was analyzed, and the nucleotide and encoded polypeptide sequences are shown in Table 26A.

TABLE 26A
NOV26 Sequence Analysis

| | |
|---|---|
| SEQ ID NO: 83 | 2897 bp |

| | |
|---|---|
| NOV26a, | GGCACGAGGGCGATGGCGACGGTCGCAGCAAATCCAGCTGCTGCTGCGGCGGCTGTGG |
| CG128021- | |
| 01 DNA | CGGCGGCAGCGGCGGTGACTGAGGATAGAGAGCCACAGCACGAGGAGCTGCCAGGCCT |
| Sequence | |
| | GGACAGCCAGTGGCGCCAGATAGAAAACGGCGAGAGTGGGCGAGAACGTCCACTGCGG |
| | GCCGGCGAAAGCTGGTTCCTTGTGGAGAAGCACTGGTATAAGCAGTGGGAGGCATACG |
| | TGCAGGGAGGGGACCAGGACTCCAGCACCTTCCCTGGCTGCATCAACAATGCCACACT |
| | CTTTCAAGATGAGATAAACTGGCGCCTCAAGGAGGGACTGGTGGAAGGCGAGGATTAT |
| | GTGCTGCTCCCAGCAGCTGCTTGGCATTACCTGGTCAGCTGGTATGGTCTAGAGCATG |
| | GCCAGCCACCCATTGAACGCAAGGTCATAGAGCTGCCCAACATCCAGAAGGTCGAAGT |
| | GTACCCAGTAGAACTGCTGCTTGTCCGGCACAATGATTTGGGCAAATCTCACACTGTT |
| | CAGTTCAGCCATACCGATTCTATTGGCCTAGTATTGCGCACAGCTCGGGAGCGGTTTC |
| | TGGTGGAGCCCCAGGAAGACACTCGGCTTTGGGCCAAGAACTCAGAAGGCTCTTTGGA |
| | TAGGTTGTATGACACACACATCACGGTTCTCGATGCGGCCCTTGAGACTGGGCAGTTG |
| | ATCATCATGGAGACCCGCAAGAAAGATGGCACTTGGCCCAGCGCACAGCTGCATGTCA |
| | TGAACAACAACATGTCGGAAGAGGATGAGGACTTCAAGGGTCAGCCAGGCATCTGTGG |
| | CCTCACCAATCTGGGCAACACGTGCTTCATGAACTCGGCCCTGCAGTGCCTCAGCAAT |
| | GTGCCACAGCTCACCGAGTACTTCCTCAACAACTGCTACCTGGAGGAGCTCAACTTCC |
| | GCAACCCACTGGGCATGAAGGGTGAGATCGCAGAGGCCTATGCAGACCTGGTGAAGCA |
| | GGCGTGGTCTGGCCACCACCGCTCCATTGTGCCACATGTGTTCAAGAACAAGGTTGGC |
| | CATTTTGCATCCCAATTTCTGGGCTACCAGCAGCATGACTCTCAGGAGCTGCTGTCAT |
| | TCCTCCTGGACGGGCTGCATGAGGACCTTAATCGGGTGAAGAAGAAGGAGTATGTGGA |
| | GCTGTGCGATGCTGCTGGGCGACCGGATCAGGAGGTGGCACAGGAGGCATGGCAAAAC |
| | CACAAACGGCGGAACGATTCTGTGATCGTGGACACTTTCCACGGCCTCTTCAAGTCCA |
| | CGCTGGTGTGCCCCGATTGTGGCAATGTATCTGTGACCTTCGACCCCTTCTGCTACCT |
| | CAGTGTTCCACTGCTTATCAGCCACAAGAGGGTCTTGGAGGTCTTCTTTATCCCCATG |
| | GATCCGCGCCGCAAGCCAGAGCAGCACCGGCTCGTGGTCCCCAAGAAAGGCAAGATCT |
| | CGGATCTATGTGTGGCTCTGTCCAAACACACGGGCATCTCGCCAGAGAGGATGATGGT |
| | GGCTGATGTCTTCAGTCACCGCTTCTATAAGCTCTATCAGCTAGAGGAGCCTCTGAGC |
| | AGCATCTTGGACCGTGATGATATCTTCGTCTATGAGGTGTCAGGTCGCATTGAGGCCA |
| | TTGAGGGCTCAAGAGAGGACATCGTGGTTCCTGTCTACCTGCGGGAGCGCACCCCTGC |
| | CCGTGACTACAACAACTCCTACTACGGCCTGATGCTTTTTGGACACCCCCTCCTGGTA |
| | TCAGTGCCCCGGGACCGCTTCACCTGGGAGGGCCTGTATAACGTCCTGATGTACCGGC |
| | TCTCACGCTACGTGACCAAACCCAACTCAGATGATGAGGACGATGGGGATGAGAAAGA |
| | AGATGACGAGGAGGATAAAGATGACGTCCCTGGGCCCTCAACTGGGGGCAGCCTCCGA |
| | GACCCTGAGCCAGAGCAGGCTGGGCCCAGCTCTGGAGTCACGAACAGGTGCCCGTTCC |
| | TCCTGGACAATTGCCTTGGCACATCTCAGTGGCCCCCAAGGCGACGACGCAAGCAGCT |
| | GTTCACCCTGCAGACGGTGAACTCCAATGGGACCAGCGACCGCACAACCTCCCCTGAA |
| | GAAGTCCATGCCCAGCCGTACATTGCTATCGACTGGGAGCCAGAGATGAAGAAGCGTT |
| | ACTATGACGAGGTAGAGGCTGAGGGCTACGTGAAGCATGACTGCGTCGGGTACGTGAT |
| | GAAGAAGGCTCCCGTGCGGCTGCAGGAGTGCATTGAGCTCTTCACCACTGTGGAGACC |
| | CTGGAGAAGGAAAACCCCTGGTACTGCCCTTCCTGCAAGCAGCACCAGCTGGCAACCA |
| | AGAAGCTGGACCTGTGGATGCTGCCGGAGATTCTCATCATCCACCTGAAACGCTTTTC |
| | CTACACCAAGTTCTCCCGAGAGAAGCTGGACACCCTCGTGGAGTTTCCTATCCGGTCA |
| | GGGGCCAGGGAGAGGATGGCTGGGGGAAGGCAGGGAAAGGAGGGGGTGTACCAGTATT |
| | AACCCTCTCCCCACCCACAGGGACCTGGACTTCTCTGAGTTTGTCATCCAGCCACAGA |
| | ATGAGTCGAATCCGGAGCTGTACAAATATGACCTCATCGCGGTTTCCAACCATTATGG |
| | GGGCATGCGTGATGGACACTACACAACATTTGCCTGCAACAAGGACAGCGGCCAGTGG |
| | CACTACTTTGATGACAACAGCGTCTCCCCTGTCAATGAGAATCAGATCGAGTCCAAGG |
| | CAGCCTATGTCCTCTTCTACCAACGCCAGGACGTGGCGCGACGCCTGCTGTCCCCGGC |
| | CGGCTCATCTGGCGCCCCAGCCTCCCCTGCCTGCAGCTCCCCACCCAGCTCTGAGTTC |
| | ATGGATGTTAATTGAGAGCCCTGGGTCCTGCCACAGAAAAAAAAAAAAAAAAAAA |

| | |
|---|---|
| ORF Start: ATG at 13 | ORF Stop: TAA at 2494 |

| | | |
|---|---|---|
| SEQ ID NO: 84 | 827 aa | MW at 94655.9 kD |

| | |
|---|---|
| NOV26a, | MATVAANPAAAAAAVAAAAAVTEDREPQHEELPGLDSQWRQIENGESGRERPLRAGES |
| CG128021- | |
| 01 Protein | WFLVEKHWYKQWEAYVQGGDQDSSTFPGCINNATLFQDEINWRLKEGLVEGEDYVLLP |
| Sequence | |
| | AAAWHYLVSWYGLEHGQPPIERKVIELPNIQKVEVYPVELLLVRHNDLGKSHTVQFSH |
| | TDSIGLVLRTARERFLVEPQEDTRLWAKNSEGSLDRLYDTHITVLDAALETGQLIIME |
| | TRKKDGTWPSAQLHVMNNNMSEEDEDFKGQPGICGLTNLGNTCFMNSALQCLSNVPQL |
| | TEYFLNNCYLEELNFRNPLGMKGEIAEAYADLVKQAWSGHHRSIVPHVFKNKVGHFAS |
| | QFLGYQQHDSQELLSFLLDGLHEDLNRVKKKEYVELCDAAGRPDQEVAQEAWQNHKRR |
| | NDSVIVDTFHGLFKSTLVCPDCGNVSVTFDPFCYLSVPLLISHKRVLEVFFIPMDPRR |
| | KPEQHRLVVPKKGKISDLCVALSKHTGISPERMMVADVFSHRFYKLYQLEEPLSSILD |
| | RDDIFVYEVSGRIEAIEGSREDIVVPVYLRERTPARDYNNSYYGLMLFGHPLLVSVPR |
| | DRFTWEGLYNVLMYRLSRYVTKPNSDDEDDGDEKEDDEEDKDDVPGPSTGGSLRDPEP |
| | EQAGPSSGVTNRCPFLLDNCLGTSQWPPRRRRKQLFTLQTVNSNGTSDRTTSPEEVHA |
| | QPYIAIDWEPEMKKRYYDEVEAEGYVKHDCVGYVMKKAPVRLQECIELFTTVETLEKE |
| | NPWYCPSCKQHQLATKKLDLWMLPEILIIHLKRFSYTKFSREKLDTLVEFPIRSGARE |
| | RMAGGRQGKEGVYQY |

Further analysis of the NOV26a protein yielded the following properties shown in Table 26B.

TABLE 26B
Protein Sequence Properties NOV26a

| | |
|---|---|
| PSort | 0.5500 probability located in endoplasmic reticulum |
| analysis: | (membrane); 0.1900 probability located in lysosome |
| | (lumen); 0.1440 probability located in nucleus; 0.1000 |
| | probability located in endoplasmic reticulum (lumen) |
| SignalP | No Known Signal Sequence Predicted |
| analysis: | |

A search of the NOV26a protein against the Geneseq database, a proprietary database that contains sequences published in patents and patent publications, yielded several homologous proteins shown in Table 26C.

TABLE 26C
Geneseq Results for NOV26a

| | | NOV26a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAU31808 | Novel human secreted protein #2299- | 22 . . . 797 | 734/786 (93%) | 0.0 |
| | Homo sapiens, 1024 aa. | 19 . . . 804 | 745/786 (94%) | |
| | [WO200179449-A2, 25 Oct. 2001] | | | |
| AAY70014 | Human Protease and associated protein- | 53 . . . 806 | 368/820 (44%) | 0.0 |
| | 8 (PPRG-8)-Homo sapiens, 952 aa. | 24 . . . 827 | 512/820 (61%) | |
| | [WO200009709-A2, 24 Feb. 2000] | | | |
| AAW54094 | Homo sapiens BE455 sequence-Homo | 634 . . . 807 | 174/174 (100%) | e−102 |
| | sapiens, 290 aa. [WO9812327-A2, | 4 . . . 177 | 174/174 (100%) | |
| | 26 Mar. 1998] | | | |
| AAU82715 | Amino acid sequence of novel human | 85 . . . 502 | 171/455 (37%) | 1e−77 |
| | protease #14-Homo sapiens, 1604 aa. | 521 . . . 969 | 251/455 (54%) | |
| | [WO200200860-A2, 3 Jan. 2002] | | | |
| AAY92344 | Human cancer associated antigen | 106 . . . 521 | 166/442 (37%) | 2e−77 |
| | precursor from clone NY-REN-60- | 18 . . . 452 | 248/442 (55%) | |
| | Homo sapiens, 462 aa. | | | |
| | [WO200020587-A2, 13 Apr. 2000] | | | |

In a BLAST search of public sequence databases, the NOV26a protein was found to have homology to the proteins shown in the BLASTP data in Table 26D.

TABLE 26D
Public BLASTP Results for NOV26a

| | | NOV25a | | |
|---|---|---|---|---|
| Protein | | Residues/ | Identities/ | |
| Accession | | Match | Similarities for | Expect |
| Number | Protein/Organism/Length | Residues | the Matched Portion | Value |
| P51784 | Ubiquitin carboxyl-terminal hydrolase 11 | 231 . . . 807 | 576/577 (99%) | 0.0 |
| | (EC 3.1.2.15) (Ubiquitin thiolesterase 11) | 1 . . . 577 | 576/577 (99%) | |
| | (Ubiquitin-specific processing protease 11) | | | |
| | (Deubiquitinating enzyme 11)-Homo | | | |
| | sapiens (Human), 690 aa. | | | |
| Q99K46 | Similar to ubiquitin specific protease 11- | 231 . . . 807 | 493/589 (83%) | 0.0 |
| | Mus musculus (Mouse), 699 aa. | 1 . . . 587 | 538/589 (90%) | |
| Q921M8 | Similar to ubiquitous nuclear protein-Mus | 45 . . . 825 | 387/840 (46%) | 0.0 |
| | musculus (Mouse), 915 aa. | 4 . . . 817 | 514/840 (61%) | |
| Q9PWC6 | Ubiquitous nuclear protein-Gallus gallus | 53 . . . 806 | 372/820 (45%) | 0.0 |
| | (Chicken), 950 aa. | 24 . . . 825 | 514/820 (62%) | |
| Q9UNP0 | Deubiquitinating enzyme-Homo sapiens | 53 . . . 806 | 369/820 (45%) | 0.0 |
| | (Human), 952 aa. | 24 . . . 827 | 513/820 (62%) | |

PFam analysis predicts that the NOV26a protein contains the domains shown in the Table 26E.

TABLE 26E
Domain Analysis of NOV26a

| | | Identities/ | |
|---|---|---|---|
| Pfam | | Similarities for | Expect |
| Domain | NOV26a Match Region | the Matched Region | Value |
| UCH-1 | 266 . . . 297 | 19/32 (59%) | 2.3e−15 |
| | | 31/32 (97%) | |

### Example 27

The NOV27 clone was analyzed, and the nucleotide and encoded polypeptide sequences are shown in Table 27A.

TABLE 27A
NOV27 Sequence Analysis

| | |
|---|---|
| SEQ ID NO: 85 | 1552 bp |

| | |
|---|---|
| NOV27a, | CAGAAATCTCAGGTCAGAGGCACGGACAGCCTCTGGAGCTCTCGTCTGGTGGGACCAT |
| CG128291- | |
| 01 DNA | GAACTGCCAGCAGCTGTGGCTGGGCTTCCTACTCCCCATGACAGTCTCAGGCCGGGTC |
| Sequence | |
| | CTGGGGCTTGCAGAGGTGGCGCCCGTGGACTACCTGTCACAATATGGGTACCTACAGA |
| | AGCCTCTAGAAGGATCTAATAACTTCAAGCCAGAAGATATCACCGAGGCTCTGAGAGC |
| | TTTTCAGGAAGCATCTGAACTTCCAGTCTCAGGTCAGCTGGATGATGCCACAAGGGCC |
| | CGCATGAGGCAGCCTCGTTGTGGCCTAGAGGATCCCTTCAACCAGAAGACCCTTAAAT |
| | ACCTGTTGCTGGGCCGCTGGAGAAAGAAGCACCTGACTTTCCGCATCTTGAACCTGCC |
| | CTCCACCCTTCCACCCCACACAGCCCGGGCAGCCCTGCGTCAAGCCTTCCAGGACTGG |
| | AGCAATGTGGCTCCCTTGACCTTCCAAGAGGTGCAGGCTGGTGCGGCTGACATCCGCC |
| | TCTCCTTCCATGGCCGCCAAAGCTCGTACTGTTCCAATACTTTTGATGGGCCTGGGAG |
| | AGTCCTGGCCCATGCCGACATCCCAGAGCTGGGCAGTGTGCACTTCGACGAAGACGAG |
| | TTCTGGACTGAGGGGACCTACCGTGGGGTGAACCTGCGCATCATTGCAGCCCATGAAG |
| | TGGGCCATGCTCTGGGGCTTGGGCACTCCCGATATTCCCAGGCCCTCATGGCCCCAGT |
| | CTACGAGGGCTACCGGCCCCACTTTAAGCTGCACCCAGATGATGTGGCAGGGATCCAG |
| | GCTCTCTATGGGCCCCGTGGGAAGACCTATGCTTTCAAGGGGGACTATGTGTGGACTG |
| | TATCAGATTCAGGACCGGGCCCCTTGTTCCGAGTGTCTGCCCTTTGGGAGGGGCTCCC |
| | CGGAAACCTGGATGCTGCTGTCTACTCGCCTCGAACACAATGGATTCACTTCTTTAAG |
| | GGAGACAAGGTGTGGCGCTACATTAATTTCAAGATGTCTCCTGGCTTCCCCAAGAAGC |
| | TGAATAGGGTAGAACCTAACCTGGATGCAGCTCTCTATTGGCCTCTCAACCAAAAGGT |
| | GTTCCTCTTTAAGGGCTCCGGGTACTGGCAGTGGGACGAGCTAGCCCGAACTGACTTC |
| | AGCAGCTACCCCAAACCAATCAAGGGTTTGTTTACGGGAGTGCCAAACCAGCCCTCGG |
| | CTGCTATGAGTTGGCAAGATGGCCGAGTCTACTTCTTCAAGGGCAAAGTCTACTGGCG |
| | CCTCAACCAGCAGCTTCGAGTAGAGAAAGGCTATCCCAGAAATATTTCCCACAACTGG |
| | ATGCACTGTCGTCCCCGGACTATAGACACTACCCCATCAGGTGGGAATACCACTCCCT |
| | CAGGTACGGGCATAACCTTGGATACCACTCTCTCAGCCACAGAAACCACGTTTGAATA |
| | CTGACTGCTCACCCACAGACACAATCTTGGACATTAACCCCTGAGGCTCCACCACCCA |
| | CCCTTTCATTTCCCCCCCAGAAGCCTAAGGCCTAATAGCTGAAT |

| | |
|---|---|
| ORF Start: ATG at 57 | ORF Stop: TGA at 1452 |

| | | |
|---|---|---|
| SEQ ID NO: 86 | 465 aa | MW at 52665.2 kD |

| | |
|---|---|
| NOV27a | MNCQQLWLGFLLPMTVSGRVLGLAEVAPVDYLSQYGYLQKPLEGSNNFKPEDITEALR |
| CG128291- | |
| 01 Protein | AFQEASELPVSGQLDDATRARMRQPRCGLEDRFNQKTLKYLLLGRWRKKHLTFRILNL |
| Sequence | |
| | PSTLPPHTARAALRQAFQDWSNVAPLTPQEVQAGAADIRLSFHGRQSSYCSNTFDGPG |
| | RVLAHADIPELGSVHFDEDEFWTEGTYRGVNLRIIAAHEVGHALGLGHSRYSQALMAP |
| | VYEGYRPHFKLHPDDVAGIQALYGPRGKTYAFKGDYVWTVSDSGPGPLFRVSALWEGL |
| | VFLFKGSGYWQWDELARTDFSSYPKPIKGLFTGVPNQPSAAMSWQDGRVYFFKGKVYW |
| | RLNQQLRVEKGYPRNISHNWMHCRPRTIDTTRSGGNTTPSGTGITLDTTLSATETTFE |
| | Y |

Further analysis of the NOV27a protein yielded the following properties shown in Table 27B.

TABLE 27B
Protein Sequence Properties NOV27a

| | |
|---|---|
| PSort | 0.8650 probability located in lysosome (lumen); 0.3700 |
| analysis: | probability located in outside; 0.2801 probability located |
| | in microbody (peroxisome); 0.1000 probability located in |
| | endoplasmic reticulum (membrane) |
| SignalP | Cleavage site between residues 19 and 20 |
| analysis: | |

A search of the NOV27a protein against the Geneseq database, a proprietary database that contains sequences published in patents and patent publication, yielded several homologous proteins shown in Table 27C.

TABLE 27C
Geneseq Results for NOV27a

| | | NOV27a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAU78837 | Human matrix metalloproteinase 19 | 1 . . . 465 | 465/508 (91%) | 0.0 |
| | (MMP-19)-Homo sapiens, 508 aa. | 1 . . . 508 | 465/508 (91%) | |
| | [WO200211530-A1, 14 Feb. 2002] | | | |
| AAB84620 | Amino acid sequence of matrix | 1 . . . 465 | 465/508 (91%) | 0.0 |
| | metalloproteinase-19-Homo sapiens, | 1 . . . 308 | 465/508 (91%) | |
| | 508 aa. [WO200149309-A2, | | | |
| | 12 Jul. 2001] | | | |
| AAE10427 | Human matrix metalloprotinase-18P | 1 . . . 465 | 465/508 (91%) | 0.0 |
| | (MMP-18P) protein-Homo sapiens, | 1 . . . 508 | 465/508 (91%) | |
| | 508 aa. [WO200166766-A2, | | | |
| | 13 Sep. 2001] | | | |
| AAW16622 | Human metalloprotease MPRS-Homo | 1 . . . 465 | 465/508 (91%) | 0.0 |
| | sapiens, 508 aa. [WO9719178-A2, | 1 . . . 508 | 465/508 (91%) | |
| | 29 May 1997] | | | |
| AAW34075 | Human liver derived metalloprotease- | 1 . . . 465 | 465/508 (91%) | 0.0 |
| | Homo sapiens, 508 aa. [WO9740157- | 1 . . . 508 | 465/508 (91%) | |
| | A1, 30 Oct. 1997] | | | |

In a BLAST search of public sequence databases, the NOV27a protein was found to have homology to the proteins shown in the BLASTP data in Table 27D.

TABLE 27D
Public BLASTP Results for NOV27a

| | | NOV27a | | |
|---|---|---|---|---|
| Protein | | Residues | Identities | |
| Accession | | Match | Similarities for the | Expect |
| Number | Protein/Organism | Residues | Matched Portion | Value |
| Q99542 | Matrix metalloproteinase-19 precursor | 1 . . . 465 | 465/508 (91%) | 0.0 |
| | (EC 3.4.24.-) (MMP-19) (Matrix | 1 . . . 508 | 465/508 (91%) | |
| | Homo sapiens (Human), 508 aa. | | | |
| Q9JH10 | Matrix metalloproteinase-19 precursor | 1 . . . 464 | 373/507 (73%) | 0.0 |
| | (EC 3.4.24.-) (MMP-19) (Matrix | 1 . . . 506 | 411/507 (80%) | |
| | metalloproteinase RASI) - Mus | | | |
| | musculus (Mouse), 527 aa. | | | |
| Q9GTK3 | Matrix metalloproteinase I - Drosophila | 20 . . . 449 | 180/503 (35%) | 3e−69 |
| | melanogaster (Fruit fly), 567 aa. | 44 . . . 527 | 242/503 (47%) | |
| AAM48434 | RE62222p - Drosophila melanogaster | 20 . . . 449 | 179/503 (35%) | 5e−69 |
| | (Fruit fly), 584 aa. | 18 . . . 501 | 242/503 (47%) | |
| Q9W122 | CG4859 protein - Drosophila | 31 . . . 449 | 175/485 (36%) | 2e−68 |
| | melanogaster (Fruit fly), 568 aa. | 20 . . . 485 | 235/485 (48%) | |

PFam analysis predicts that the NOV27a protein contains the domains shown in the Table 27E.

TABLE 27E
Domain Analysis of NOV27a

| | NOV27a | Identities/Similarities | Expect |
|---|---|---|---|
| Pfam Domain | Match Region | for the Matched Region | Value |
| Peptidase₋M10 | 31 . . . 197 | 67/176 (38%) | 1.7e−26 |
| | | 119/176 (68%) | |
| Hemopexin | 251 . . . 292 | 20/50 (40%) | 0.0017 |
| | | 30/50 (60%) | |
| Hemopexin | 294 . . . 335 | 15/50 (30%) | 0.00014 |
| | | 34/50 (68%) | |
| Hemopexin | 337 . . . 384 | 17/50 (34%) | 2e−08 |
| | | 36/50 (72%) | |
| Hemopexin | 386 . . . 429 | 20/50 (40%) | 1.1e−11 |
| | | 34/50 (68%) | |

### Example 28

The NOV28 clone was analyzed, and the nucleotide and encoded polypeptide sequences are shown in Table 28A.

TABLE 28A
NOV28 Sequence Analysis

| | |
|---|---|
| SEQ ID NO: 87 | 4487 bp |

| | |
|---|---|
| NOV28a, | CCGCGTCCGCGGACGCGTGGGGGCGAGGGCCGCTGGGGCCGCGAAGTGGGGCGGCCGG |
| CG128380- | |
| 01 DNA | GTGGGCTACGAGCCGGGTCTGGGCTGAGGGGCGCGCCTTCGCCGTGGACCCCAGCCCG |
| Sequence | |
| | GCAACGGGAAGGCGAGCTCTCCTCCACCGTCCAAAGTAAACTTTGCCGCTCCTTCCGC |
| | GGCGCTCCCGAGTCCTCGCCGCCGCCGGGCCGCCGCAGTCCGCGAAGAGCCGTCCTGC |
| | GTCAGGGCCTCCTTCCCTGCCCCGGCGCGGGGCCACTGCGCCATGGACGCCACAGCAC |
| | TGGAGCGGGACGCTGTGCAGTTCGCCCGTCTGGCGGTTCAGCGCGACCACGAAGGCCG |
| | CTACTCCGAGGCGGTGTTTTATTACAAGGAAGCTGCACAAGCCTTAATTTATGCTGAG |
| | ATGGCAGGATCAAGCCTAGAAAATATTCAAGAAAAAATAACTGAGTATCTGGAAAGAG |
| | TTCAAGCTCTACATTCAGCAGTTCAGTCAAAGAGTGCTGATCCTTTGAAGTCAAAACA |
| | TCAGTTGGACTTAGAGCGTGCTCATTTCCTTGTTACACAAGCTTTTGATGAAGATGAA |
| | AAAGAGAATGTTGAAGATGCTATAGAATTGTACACAGAAGCTGTGGATCTCTGTCTGA |
| | AAACATCTTATGAAACTGCTGATAAAGTCCTGCAAAATAAACTGAAACAGTTGGCTCG |
| | ACAGGCACTAGACAGAGCAGAAGCGCTGAGTGAGCCTTTGACCAAGCCAGTTGGCAAA |
| | ATCAGTTCAACAAGTGTTAAGCCAAAGCCACCTCCAGTGAGAGCACATTTTCCACTGG |
| | GCGCTAATCCCTTCCTTGAAAGACCTCAGTCATTTATAAGTCCTCAGTCATGTGATGC |
| | ACAAGGACAGAGATACACAGCAGAAGAAATAGAAGTACTCAGGACAACATCAAAAATA |
| | AATGGTATAGAATATGTTCCTTTCATGAATGTTGACCTGAGAGAACGTTTTGCCTATC |
| | CAATGCCTTTCTGTGATAGATGGGGCAAGCTACCATTATCACCTAAACAAAAAACTAC |
| | ATTTTCCAAGTGGGTACGACCAGAAGACCTCACCAACAATCCTACAATGATATATACT |
| | GTGTCCAGTTTTAGCATAAAGCAGACAATAGTATCGGATTGCTCCTTTGTGGCATCAC |
| | TGGCCATCAGTGCAGCTTATGAAAGACGTTTTAATAAGAAGTTAATTACCGGCATAAT |
| | TTACCCTCAAAACAAGGATGGTGAACCAGAATACAATCCATGTGGGAAGTATATGGTA |
| | AAACTTCACCTCAATGGTGTCCCAAGAAAGGTGATAATTGATGACCAGTTACCTGTTG |
| | ATCACAAGGGAGAATTGCTCTGTTCTTATTCCAACAACAAAAGTGAATTATGGGTTTC |
| | TCTCATAGAAAAAGCATACATGAAAGTCATGGGAGGATATGATTTTCCAGGATCCAAC |
| | TCCAATATTGATCTTCATGCACTGACTGGCTGGATACCAGAAAGAATTGCTATGCATT |
| | CAGATAGCCAAACTTTCAGTAAGGATAATTCTTTCAGAATGCTTTATCAAAGATTTCA |
| | CAAAGGAGATGTCCTCATCACTGCGTCAACTGGAATGATGACAGAAGCTGAAGGAGAG |
| | AAGTGGGGTCTGGTTCCCACACACGCATATGCTGTTTTGGATATTAGAGAGTTCAAGG |
| | GGCTGCGATTTATCCAGTTGAAAAATCCTTGGAGTCATTTACGTTGGAAAGGAAGATA |
| | CAGTGAAAATGATGTAAAAAACTGGACTCCAGAGTTGCAAAAGTATTTAAACTTTGAT |
| | CCCCGAACAGCTCAGAAAATAGACAACGGAATATTTTGGATTTCCTGGGATGATCTCT |
| | GCCAGTATTATGATGTGATTTATTTGAGTTGGAATCCAGGTCTTTTTAAAGAATCAAC |
| | ATGTATTCACAGTACTTGGGATGCTAAGCAAGGACCTGTGAAAGATGCCTATAGCCTG |
| | GCCAACAACCCCCAGTACAAACTGGAGGTGCAGTGTCCACAGGGGGGTGCTGCAGTTT |
| | GGGTTTTGCTTAGTAGACACATAACAGACAAGGATGATTTTGCGAATAATCGAGAATT |
| | TATCACAATGGTTGTATACAAGACTGATGGAAAAAAAGTTTATTACCCAGCTGACCCA |
| | CCTCCATACATTGATGGAATTCGAATTAACAGCCCTCATTATTTGACTAAGATAAAGC |
| | TGACCACACCTGGCACCCATACCTTTACATTAGTGGTTTCTCAATATGAAAAACAGAA |
| | CACAATCCATTACACGGTTCGGGTATATTCAGCATGCAGCTTTACTTTTTCAAAGATT |
| | CCTTCACCATACACCTTATCAAAACGGATTAATGGAAAGTGGAGTGGTCAGAGTGCTG |
| | GAGGATGTGGAAATTTCCAAGAGACTCACAAAAATAACCCCATCTACCAATTCCATAT |
| | AGAAAAGACTGGGCCGTTACTGATTGAGCTACGAGGACCAAGGAGATCCTGGTCCCCA |
| | TGGCTTTCTGAGGAAATCTAGTGGTGACTATAGGTGTGGGTTTTGCTACCTGGAATTA |
| | GAAATATACCTTCTGGGATCTTCAATATCATTCCTAGTACCTTTTTGCCTAAACAAGA |
| | AGGACCTTTTTTCTTGGACTTTAATAGTATTATCCCCATCAAGATCACACAACTTCAG |
| | TGATGGAGAAATCTCAAGTTACTGGCTTTTATACTTACCAAACATCAGTTCTTCAAAT |
| | AAGGACGCAAATCTTCAGGACAGTAAGCAGAACAATCAGAATGGAATTAAATCTCTAA |
| | AAACGTGTTACAGTGGAATCTGGTGCTTGTCAGGGTGTTTGGTAAGAACTGTATATAG |
| | TCAGAATTACCTAAATCACCTAGAGGTACCGTTTACATGGTTTTGTGTATATAGAGTT |
| | GGCTTGCATTTTAGGGGCCATTTTGTATAAAAAGTGCATATGATTAAAATTAGACTCA |
| | GTCATCACTGTGAGATGCCTTTGCTAAGAGGATAAAGGAACTGAGACCAGATGAGAAA |
| | AAGAAAGGATATAGATTCCTTGAGTGGAATAGTGGGCTAGATTAATATACCGAAATAT |
| | TTCCATTGTTTCCCTTTTTTGCAGAGCATGTGGAAGTTAAACCTGCTTGATTCTACTA |
| | TACATCTTGGGCAACTAGTTACCAAATGAATTGTGCCACCATAACTGATTTTAATTTT |
| | GCATTATTTATGATTTTAAAATATTTGTTGCCCAGGTGTTATGAAAGAATAAACCTTT |
| | TAAGTATAGACTACCTTAGCATGAAGATGCTCATGCCTAAGAATGAAAATTGTTGAGG |
| | TTATCTCCCATTCAATCATGTAGCAAGAACTTAAAGAAATTCACTACTGCAGTTTTTA |
| | TTTTTAAAAAACAGTAATTGAGATATTGAAGACATTACAATTTAGTTTGTGTGGTCTT |
| | TTTTTAAATTGCTGTATCGTTCAGTCTCTTGTGGCAATAGCACTTTGAAGAAAATAGA |
| | GAATTTAATATATGGTGATTGGGATATGTAGCATTCAAAAAAAGTGAATTGCCAAGAT |
| | ACTGGTGTCATGTAAATTCCCACTTTACATAAAAACCCATCAGGACAGAATGATGCTC |
| | AATATTTTAAAATTCTAAAAATAGGGTGGGATTTTTCATTGTCTCTACTTTATAATTA |
| | TCAAAACTTATTTTGTATTGCTACTACCTTAAATTGAAATAAAATGTTTATACTTACG |
| | GATATTGCATAGTTTAAGTTAGATTTATTGAAAGATTTCATCTGTCGTGTTTCATGTA |
| | AATGAGAACAGATTATTTGCATGAAAATATATACTTCAACAAAAATCTGTTCTTTAAC |
| | AGAGTAGTGGTAGATTATTACACTAATGAGATTTCACTTTGGTAAATACTTCATGCTT |
| | TCAGTTTTAGCCTATTAATTTTAGGTGGACAAATTTAACAAGTTTTCTGTTACTTTTT |
| | AAAAAGAAAAAATCCAGAACATAAGAACTATATTATGAACACATGATTTGAACCTGTT |
| | GTGGTAAAGATCTTGTACAGGATGCAAACTAAAAACCTAATCCCTGCCATCAAATTTA |
| | TTAGAAGAGACCTATATATGAACAACTTAAAGGCACTGATTTCTATAATAGAGCTCTA |
| | AAAACATGCCACCAGTGTATGAATAAGGGAAAGATTAATTTTGGCTGGACCAATATAA |
| | AAAATTGTATTTGAAGAATTGATACTTTAACTTGGACCTTGAAGGTAAAGCTTCAAAA |
| | GACAGGTTACTGACCATTGAGTGTTTACTATGTACCCAATGTGTATATTTTTCTTTTT |
| | AATCTTCCCAATAGCTGAATAAAGTATAGATACTATTATTTGTACTTCTTACAATTGA |
| | GGAAATAAGCCTAAGAGATTAAAAGATTTTGCCCAGGGTTCACAAGCCTTCTTCCCTG |
| | AGCCCTGATTGAGCTGCTGTGTGTGTCTAATGGCACCCACAGTCACGGCCGTCTAGTC |
| | GAGGGAGGGACAAGATCTAGA |

| | |
|---|---|
| ORF Start: ATG at 275 | ORF Stop: TAG at 2513 |

| | | |
|---|---|---|
| SEQ ID NO: 88 | 746 aa | MW at 85355.1 kD |

| | |
|---|---|
| NOV28a | MDATALERDAVQFARLAVQRDHFGRYSEAVFYYKEAAQALIYAEMAGSSLENIQEKIT |
| CG128380- | |
| 01 Protein | EYLERVQALHSAVQSKSADPLKSKHQLDLERAHFLVTQAFDEDFKENVEDAIELYTEA |
| Sequence | |
| | VDLCLKTSYETADKVLQNKLKQLARQALDRAEALSEPLTKPVGKISSTSVKPKPPPVR |
| | AHFPLGANPFLERPQSFISPQSCDAQGQRYTAEEIEVLRTTSKINGIEYVPFMNVDLR |
| | ERFAYPMPFCDRWGKLRLSPKQKTTFSKWVRPEDLTNNPTMIYTVSSFSIKQTIVSDC |
| | SFVASLAISAAYERRFNKKLITGIIYPQNKDGEPEYNPCGKYMVKLHLNGVPRKVIID |
| | DQLPVDHKGELLCSYSNNKSELWVSLIEKAYMKVMGGYDFPGSNSNIDLHALTGWIPE |
| | RIAMHSDSQTFSKDNSFRMLYQRFHKGDVLITASTGMMTEAEGEKWGLVPTHAYAVLD |
| | IREFKGLRFIQLKNPWSHLRWKGRYSENDVKNWTPELQKYLNFDPRTAQKIDNGIFWI |
| | SWDDLCQYYDVIYLSWNPGLFKESTCIHSTWDAKQGPVKDAYSLANNPQYKLEVQCPQ |
| | GGAAVWVLLSRHITDKDDFANNREFITMVVYKTDGKKVYYPADPPPYIDGIRINSPHY |
| | LTKIKLTTPGTHTFTLVVSQYEKQNTIHYTVRVYSACSFTFSKIPSPYTLSKRINGKW |
| | SGQSAGGCGNFQETHKNNPIYQFHIEKTGPLLIELRGPRRSWSPWLSEEI |

Further analysis of the NOV28a protein yielded the following properties shown in Table 28B.

TABLE 28B
Protein Sequence Properties NOV28a

| | |
|---|---|
| PSort | 0.5736 probability located in mitochondrial matrix space; |
| analysis: | 0.5077 probability located in microbody (peroxisome); 0.2872 |
| | probability located in mitochondrial inner membrane; 0.2872 |
| | probability located in mitochondrial intermembrane space |
| SignalP | No Known Signal Sequence Predicted |
| analysis: | |

A search of the NOV28a protein against the Geneseq database, a proprietary database that contains sequences published in patents and patent publication, yielded several homologous proteins shown in Table 28C.

TABLE 28C
Geneseq Results for NOV28a

| | | NOV28a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAB67649 | Amino acid sequence of a human calpain | 1 . . . 736 | 735/736 (99%) | 0.0 |
| | protease designated 26176-Homo | 1 . . . 736 | 736/736 (99%) | |
| | sapiens, 813 aa. (WO200118216-A2, | | | |
| | 15 Mar. 2001] | | | |
| AAG04040 | Human secreted protein, SEQ ID NO. | 608 . . . 746 | 138/139 (99%) | 5e−80 |
| | 8121-Homo sapiens, 139 aa. | 1 . . . 139 | 138/139 (99%) | |
| | [EP1033401-A2, 6 Sep. 2000] | | | |
| ABB05604 | Mutant Aspergillus oryzae DEBY10.3 | 205 . . . 734 | 187/556 (33%) | 8e−74 |
| | protein SEQ ID NO: 17-Aspergillus | 104 . . . 632 | 279/556 (49%) | |
| | oryzae, 854 aa. [U.S. Pat. No. 6323002- | | | |
| | B1, 27 Nov. 2001] | | | |
| AAY97155 | PalB polypeptide of Aspergillus oryzae- | 205 . . . 734 | 187/556 (33%) | 8e−74 |
| | Aspergillus oryzae, 854 aa. | 104 . . . 632 | 279/556 (49%) | |
| | [WO200046375-A2, 10 Aug. 2000] | | | |
| AAY39872 | A. oryzae DEBY10.3 locus protein | 205 . . . 734 | 187/556 (33%) | 8e−74 |
| | sequence-Aspergillus oryzae, 854 aa. | 104 . . . 632 | 279/556 (49%) | |
| | [U.S. Pat. No. 5958727-A, 28 Sep. 1999] | | | |

In a BLAST search of public sequence databases, the NOV28a protein was found to have homology to the proteins shown in the BLASTP data in Table 28D.

TABLE 28D
Public BLASTP Results for NOV28a

| | | NOV28a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9Y6W3 | PalBH (EC 3.4.22.17)-Homo | 1 . . . 736 | 735/736 (99%) | 0.0 |
| | sapiens (Human), 813 aa | 1 . . . 736 | 736/736 (99%) | |
| Q9R1S8 | PalBH (EC 3.4.22.17)-Mus | 1 . . . 736 | 704/736 (95%) | 0.0 |
| | musculus (Mouse), 813 aa. | 1 . . . 736 | 719/736 (97%) | |
| Q9Z0P9 | Capn7-Mus musculus (Mouse), | 45 . . . 736 | 661/692 (95%) | 0.0 |
| | 769 aa. | 1 . . . 692 | 675/692 (97%) | |
| Q22143 | T04A8.16 protein- | 1 . . . 698 | 310/711 (43%) | e−167 |
| | Caenorhabditis elegans, 805 aa. | 1 . . . 701 | 435/711 (60%) | |
| Q9Y6Z8 | Calpain-like protease PALBORY | 205 . . . 734 | 187/556 (33%) | 2e−73 |
| | Aspergillus oryzae, 854 aa. | 104 . . . 632 | 279/556 (49%) | |

PFam analysis predicts that the NOV28a protein contains the domains shown in the Table 28E.

TABLE 28E
Domain Analysis of NOV28a

| | | Identities/ | |
|---|---|---|---|
| Pfam | | Similarities for | Expect |
| Domain | NOV28a Match Region | the Matched Region | Value |
| Peptidase_C2 | 231 . . . 537 | 82/353 (23%) | 2.4e−15 |
| | | 177/353 (50%) | |

### Example 29

The NOV29 clone was analyzed, and the nucleotide and encoded polypeptide sequences are shown in Table 29A.

TABLE 29A
NOV29 Sequence Analysis

| | |
|---|---|
| SEQ ID NO: 89 | 1323 bp |

| | |
|---|---|
| NOV29a, | ATGAGCAACTCCGTTCCTCTGCTCTGTTTCTGGAGCCTCTGCTATTGCTTTGCTGCGG |
| CG128439- | |
| 02 DNA | GGAGCCCCGTACCTTTTGGTCCAGAGGGACGGCTGGATGATAAGCTCCACAAACCCAA |
| Sequence | |
| | AGCTACACAGACTGAGGTCAAACCATCTGTGAGGTTTAACCTCCGCACCTCCAAGGAC |
| | CCAGAGCATGAAGGATGCTACCTCTCCGTCGGCCACAGCCAGCCCTTAGAAGACTGCA |
| | GTTTCAACATGACAGCTAAAACCTTTTTCATCATTCACGGATGGACGGAGAAGGACGA |
| | TTTTTCTCTCGGGAATGTCCACTTGATCGGCTACAGCCTCGGAGCGCACGTGGCCGGG |
| | TATGCAGGCAACTTCGTGAAAGGAACGGTGGGCCGAATCACAGGTTTGGATCCTGCCG |
| | GGCCCATGTTTGAAGGGGCCGACATCCACAAGAGGCTCTCTCCGGACGATGCAGATTT |
| | TGTGGATGTCCTCCACACCTACACGCGTTCCTTCGGCTTGAGCATTGGTATTCAGATG |
| | CCTGTGGGCCACATTGACATCTACCCCAATGGGGGTGACTTCCAGCCAGGCTGTGGAC |
| | TCAACGATGTCTTGGGATCAATTGCATATGGAACAATCACAGAGGTGGTAAAATGTGA |
| | GCATGAGCGAGCCGTCCACCTCTTTGTTGACTCTCTGGTGAATCAGGACAAGCCGAGT |
| | TTTGCCTTCCAGTGCACTGACTCCAATCGCTTCAAAAAGGGGATCTGTCTGAGCTGCC |
| | GCAAGAACCGTTGTAATAGCATTGGCTACAATGCCAAGAAAATGAGGAACAAGAGGAA |
| | CAGCAAAATGTACCTAAAAACCCGGGCAGGCATGCCTTTCAGAGTTTACCATTATCAG |
| | ATGAAAATCCATGTCTTCAGTTACAAGAACATGGGAGAAATTGAGCCCACCTTTTACG |
| | TCACCCTTTATGGCACTAATGCAGATTCCCAGACTCTGCCACTGGAAATAGTGGAGCG |
| | GATCGAGCAGAATGCCACCAACACCTTCCTGGTCTACACCGAGGAGGACTTGGGAGAC |
| | CTCTTGAAGATCCAGCTCACCTGGGAGGGGGCCTCTCAGTCTTGGTACAACCTGTGGA |
| | AGGAGTTTCGCAGCTACCTGTCTCAACCCCGCAACCCCGGACGGGAGCTGAATATCAG |
| | GCGCATCCGGGTGAAGTCTGGGGAAACCCAGCGGAAACTGACATTTTGTACAGAAGAC |
| | CCTGAGAACACCAGCATATCCCCAGGCCGGGAGCTCTGGTTTCGCAAGTGTCGGGATG |
| | GCTGGAGGATGAAAAACGAAACCAGTCCCACTGTGGAGCTTCCCTGA |

| | |
|---|---|
| ORF Start: ATG at 1 | ORF Stop: TGA at 1321 |

| | | |
|---|---|---|
| SEQ ID NO:90 | 440 aa | MW at 49902.3 kD |

| | |
|---|---|
| NOV29a, | MSNSVPLLCFWSLCYCFAAGSPVPFGPEGRLEDKLHKPKATQTEVKPSVRFNLRTSKD |
| CG128439- | |
| 02 Protein | PEHEGCYLSVGHSQPLEDCSFNMTAKTFFIIHGWTEKDDFSLGNVHLIGYSLGAHVAG |
| Sequence | |
| | YAGNFVKGTVGRITGLDPAGPMFEGADIHKRLSPDDADFVDVLHTYTRSFGLSIGIQM |
| | PVGHIDIYPNGGDFQPGCGLNDVLGSIAYGTITEVVKCEHERAVHLFVDSLVNQDKPS |
| | FAFQCTDSNRFKKGICLSCRKNRCNSIGYNAKKMRNKRNSKMYLKTRAGMPFRVYHYQ |
| | MKIHVFSYKNMGEIEPTFYVTLYGTNADSQTLPLEIVERIEQNATNTFLVYTEEDLGD |
| | LLKIQLTWEGASQSWYNLWKEFRSYLSQPRNPGRELNIRRIRVKSGETQRKLTFCTED |
| | PENTSISPGRELWFRKCRDGWRMKNETSPTVELP |

| | |
|---|---|
| SEQ ID NO 91 | 608 bp |

| | |
|---|---|
| NOV29b, | ATGAGCAACTCCGTTCCTCTGCTCTGTTTCTGGAGCCTCTGCTATTGCTTTGCTGCGG |
| 171826603 | |
| DNA | GGAGCCCCGTACCTTTTGGTCCAGAGGGACGGCTGGAAGATAAGCTCCACAAACCCAA |
| Sequence | |
| | AGCTACACAGACTGAGGTCAAACCATCTGTGAGGTTTAACCTCCGCACCTCCAAGGAC |
| | CCAGAGCATGAAGGATGCTACCTCTCCGTCGGCCACAGCCAGCCCTTAGAAGACTGCA |
| | GTTTCAACATGACAGCTAAAACCTTTTTCATCATTCACGGATGGACGGAGAAGGACGA |
| | TTTTTCTCTCGGGAATGTCCACTTGATCGGCTACAGCCTCGGAGCGCACGTGGCCGGG |
| | TATGCAGGCAACTTCGTGAAAGGAACGGTGGGCCGAATCACAGGTTTGGATCCTGCCG |
| | GGCCCATGTTTGAAGGGGCCGACATCCACAAGAGGCTCTCTCCGGACGATGCAGATTT |
| | TGTGGATGTCCTCCACACCTACACGCGTTCCTTCGGCTTGAGCATTGGTATTCAGATG |
| | CCTGTGGGCCACATTGACATCTACCCCAATGGGGGTGACTTCCAGCCAGGCTGTGGAC |
| | TCAACGATGTCTTGGGATCAATTGCCTA |

| | |
|---|---|
| ORF Start: ATG at 1 | ORF Stop: at 607 |

| | | |
|---|---|---|
| SEQ ID NO:92 | 202 aa | MW at 21878.6 kD |

| | |
|---|---|
| NOV29b, | MSNSVPLLCFWSLCYCFAAGSPVRFGPEGRLEDKLHKPKATQTEVKPSVRFNLRTSKD |
| 171826603 | |
| Protein | PEHEGCYLSVGHSQPLEDCSFNMTAKTFFIIHGWTEKDDFSLGNVHLIGYSLGAHVAG |
| Sequence | |
| | YAGNFVKGTVGRITGLDPAGPMFEGADIHKRLSPDDADFVDVLHTYTRSFGLSIGIQM |
| | PVGHIDIYPNGGDFQPGCGLNDVLGSIA |

Sequence comparison of the above protein sequences yields the following sequence relationships shown in Table 29B.

TABLE 29B
Comparison of NOV29a against NOV29b

| | | Identities/ |
|---|---|---|
| Protein | NOV29a Residues/ | Similarities for |
| Sequence | Match Residues | the Matched Region |
| NOV29b | 1 . . . 202 | 202/202 (100%) |
| | 1 . . . 202 | 202/202 (100%) |

Further analysis of the NOV29a protein yielded the following, properties shown in Table 29C.

TABLE 29C
Protein Sequence Properties NOV29a

| | |
|---|---|
| PSort | 0.3700 probability located in outside; 0.1900 probability |
| analysis: | located in lysosome (lumen); 0.1800 probability located in |
| | nucleus; 0.1213 probability located in microbody |
| | (peroxisome) |
| SiginalP: | Cleavage site between residues 21 and 22 |
| analysis: | |

A search of the NOV29a protein against the Geneseq database, a proprietary database that contains sequences published in patents and patent publication, yielded several homologous proteins shown in Table 29D.

TABLE 29D
Geneseq Results for NOV29a

| | | NOV29a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAO14635 | Human lipase endothelial (LIPG) | 1 . . . 440 | 431/500 (86%) | 0.0 |
| | protein-Homo sapiens, 500 aa. | 1 . . . 500 | 435/500 (86%) | |
| | [WO200216397-A2, 28 Feb. 2002] | | | |
| AAB19178 | Human LIPG, a triacylglycerol lipase | 1 . . . 440 | 431/500 (86%) | 0.0 |
| | enzyme designated LLGXL-Homo | 1 . . . 500 | 435/500 (86%) | |
| | sapiens, 500 aa. [WO200057837-A2, | | | |
| | 5 Oct. 2000] | | | |
| AAY23759 | Human endothelial cell lipase protein | 1 . . . 440 | 431/500 (86%) | 0.0 |
| | sequence-Homo sapiens, 500 aa. | 1 . . . 500 | 435/500 (86%) | |
| | [WO9932611-A1, 1 Jul. 1999] | | | |
| AAW59792 | Amino acid sequence of lipase like | 1 . . . 440 | 431/500 (86%) | 0.0 |
| | protein LLGXL-Homo sapiens, | 1 . . . 500 | 435/500 (86%) | |
| | 500 aa. [WO9824888-A2, | | | |
| | 11 Jun. 1998] | | | |
| AAY23760 | Mouse endothelial cell lipase protein | 1 . . . 439 | 341/499 (68%) | 0.0 |
| | sequence-Mus sp, 500 aa. | 1 . . . 499 | 383/499 (76%) | |
| | [WO9932611-A1, 1 Jul. 1999] | | | |

In a BLAST search of public sequence databases, the NOV29a protein was found to have homology to the proteins shown in the BLASTP data in Table 29E.

TABLE 29E
Public BLASTP Results for NOV29a

| | | NOV29a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9Y5X9 | Endothelial lipase-Homo sapiens | 1 . . . 440 | 431/500 (86%) | 0.0 |
| | (Human), 500 aa. | 1 . . . 500 | 435/500 (86%) | |
| QSVDU2 | Lipase, endothelial-Mus | 1 . . . 439 | 343/499 (68%) | 0.0 |
| | musculus (Mouse), 500 aa. | 1 . . . 499 | 384/499 (76%) | |
| Q9WVG5 | Endothelial lipase-Mus | 1 . . . 439 | 341/499 (68%) | 0.0 |
| | musculus (Mouse), 500 aa. | 1 . . . 499 | 383/499 (76%) | |
| Q98U13 | Lipoprotein lipase-Pagrus major | 94 . . . 435 | 187/347 (53%) | e−107 |
| | (Red sea bream) (Chrysophrys | 160 . . . 503 | 252/347 (71%) | |
| | major), 511 aa. | | | |
| Q98U12 | Lipoprotein lipase-Pagrus major | 94 . . . 439 | 188/351 (53%) | e−106 |
| | (Red sea bream) (Chrysophrys | 160 . . . 507 | 253/351 (71%) | |
| | major), 510 aa. | | | |

PFam analysis predicts that the NOV29a protein contains the domains shown in the Table 29F.

TABLE 29F
Domain Analysis of NOV29a

| | | Identities/ | |
|---|---|---|---|
| Pfam | NOV29a | Similarities for | Expect |
| Domain | Match Region | the Matched Region | Value |
| Lipase | 21 . . . 284 | 114/379 (30%) | 9.4e−71 |
| | | 209/379 (55%) | |
| Chitin_synth | 361 . . . 370 | 6/10 (60%) | 0.85 |
| | | 9/10 (90%) | |
| PLAT | 287 . . . 423 | 26/147 (18%) | 4.2e−26 |
| | | 110/147 (75%) | |

### Example 30

The NOV30 clone was analyzed, and the nucleotide and encoded polypeptide sequences are shown in Table 30A.

TABLE 30A
NOV30 Sequence Analysis

| | |
|---|---|
| SEQ ID NO: 93 | 3067 bp |

| | |
|---|---|
| NOV30a, | AAGGCAATTAAGGCGCCCATTTCAGAAGAGTTACAGCCGTGAAAATTACTCAGCAGTG |
| CG128489- | |
| 01 DNA | CAGTTGGCTGAGAAGAGGAAAAAAGGTCAGGTTGTAAAGCTTTTTATTTTTCCATTTT |
| Sequence | |
| | CTAAGAGAAATTCATCATTGGAACTTGTAAAGTGGCCCAAGAGTGGCTGTAATTTGGG |
| | CCATTATAGCAGGTATGGGTGGCGTCTCTCAGCAAAGCTGACTGACTGACTGATGAGT |
| | GCTGTTTGCAATGACCTCCGCTGGAACATAATGAGAGCGCTCGCTGTGCTGTCTGTCA |
| | CGCTGGTTATGGCCTGCACAGAAGCCTTCTTCCCCTTCATCTCGAGAGGGAAAGAACT |
| | CCTTTGGGGAAAGCCTGAGGAGTCTCGTGTCTCTAGCGTCTTGGAGGAAAGCAAGCGC |
| | CTGGTGGACACCGCCATGTACGCCACGATGCAGAGAAACCTCAAGAAAAGAGGAATCC |
| | TTTCTCCAGCTCAGCTTCTGTCTTTTTCCAAACTTCCTGAGCCAACAAGCGGAGTGAT |
| | TGCCCGAGCAGCAGAGATAATGGAAACATCAATACAAGCGATGAAAAGAAAAGTCAAC |
| | CTGAAAACTCAACAATCACAGCATCCAACGGATGCTTTATCAGAAGATCTGCTGAGCA |
| | TCATTGCAAACATGTCTGGATGTCTCCCTTACATGCTGCCCCCAAAATGCCCAAACAC |
| | TTGCCTGGCGAACAAATACAGGCCCATCACAGGAGCTTGCAACAACAGAGACCACCCC |
| | AGATGGGGCGCCTCCAACACGGCCCTGGCACGATGGCTCCCTCCAGTCTATGAGCACG |
| | GCTTCAGTCAGCCCCGAGGCTGGAACCCCGGCTTCTTGTACAACGGGTTCCCACTGCC |
| | CCCGGTCCGGGAGGTGACAAGACATGTCATTCAAGTTTCAAATGAGGTTGTCACAGAT |
| | GATGACCGCTATTCTGACCTCCTGATGGCATGGGGACAATACATCGACCACGACATCG |
| | CGTTCACACCACAGAGCACCAGCAAAGCTGCCTTCGGGGGAGGGGCTGACTGCCAGAT |
| | GACTTGTGAGAACCAAAACCCATGTTTTCCCATACAACTCCCGGAGGAGGCCCGGCCG |
| | GCCGCGGGCACCGCCTGTCTGCCCTTCTACCGCTCTTCGGCCGCCTGCGGCACCGGGG |
| | ACCAAGGCGCGCTCTTTGGGAACCTGTCCACGGCCAACCCGCGGCAGCAGATGAACGG |
| | GTTGACCTCGTTCCTGGACGCGTCCACCGTGTATGGCAGCTCCCCGGCCCTAGAGAGG |
| | CAGCTGCGGAACTGGACCAGTGCCGAAGGGCTGCTCCGCGTCCACGCGCGCCTCCGGG |
| | ACTCCGGCCGCGCCTACCTGCCCTTCGTGCCGCCACGCGCGCCTTCGGCCTGTGCGCC |
| | CGAGCCCGGCATCCCCGGAGAGACCCGCGGGCCCTGCTTCCTGGCCGGAGACGGCCGC |
| | GCCAGCGAGGTCCCCTCCCTGACGGCACTGCACACGCTGTGGCTGCGCGAGCACAACC |
| | GCCTGGCCGCGGCGCTCAAGGCCCTCAATGCGCACTGGAGCGCGGACGCCGTGTACCA |
| | GGAGGCGCGCAAGGTCGTGGGCGCTCTGCACCAGATCATCACCCTGAGGGATTACATC |
| | CCCAGGATCCTGGGACCCGAGGCCTTCCAGCAGTACGTGGGTCCCTATGAAGGCTATG |
| | ACTCCACCGCCAACCCCACTGTGTCCAACGTGTTCTCCACAGCCGCCTTCCGCTTCGG |
| | CCATGCCACGATCCACCCGCTGGTGAGGAGGCTGGACGCCAGCTTCCAGGAGCACCCC |
| | GACCTGCCCGGGCTGTGGCTGCACCAGGCTTTCTTCAGCCCATGGACATTACTCCGTG |
| | GAGGTTACAATGAGTGGAGGGAGTTCTGCGGCCTGCCTCGCCTGGAGACCCCCGCTGA |
| | CCTGAGCACAGCCATCGCCAGCAGGAGCGTGGCCGACAAGATCCTGGACTTGTACAAG |
| | CATCCTGACAACATCGATGTCTGGCTGGGAGGCTTAGCTGAAAACTTCCTCCCCAGGG |
| | CTCGGACAGGGCCCCTGTTTGCCTGTCTCATTGGGAAGCAGATGAAGGCTCTGCGGGA |
| | TGGTGACTGGTTTTGGTGGGAGAACAGCCACGTCTTCACGGATGCACAGAGGCGTGAG |
| | CTGGAGAAGCACTCCCTGTCTCGGGTCATCTGTGACAACACTGGCCTCACCAGGGTGC |
| | CCATGGATGCCTTCCAAGTCGGCAAATTCCCCGAAGACTTTGAGTCTTGTGACAGCAT |
| | CCCTGGCATGAACCTGGAGGCCTGGAGGGAAACCTTTCCTCAAGACGACAAGTGTGGC |
| | TTCCCAGAGAGCGTGGAGAATGGGGACTTTGTGCACTGTGAGGAGTCTGGGAGGCGCG |
| | TGCTGGTGTATTCCTGCCGGCACGGGTATGAGCTCCAAGGCCGGGAGCAGCTCACTTG |
| | CACCCAGGAAGGATGGGATTTCCAGCCTCCCCTCTGCAAAGATGTGAACGAGTGTGCA |
| | GACGGTGCCCACCCCCCCTGCCACGCCTCTGCGAGGTGCAGAAACACCAAAGGCGGCT |
| | TCCAGTGTCTCTGCGCGGACCCCTACGAGTTAGGAGACGATGGGAGAACCTGCGTAGA |
| | CTCCGGGAGGCTCCCTCGGGCGACTTGGATCTCCATGTCGCTGGCTGCTCTGCTGATC |
| | GGAGGCTTCGCAGGTCTCACCTCGACGGTGATTTGCAGGTGGACACGCACTGGCACTA |
| | AATCCACACTGCCCATCTCGGAGACAGGCGGAGGAACTCCCGAGCTGAGATGCGGAAA |
| | GCACCAGGCCGTAGGGACCTCACCGCAGCGGGCCGCAGCTCAGGACTCGGAGCAGGAG |
| | AGTGCTGGGATGGAAGGCCGGGATACTCACAGGCTGCCGAGAGCCCTCTGAGGGCAAA |
| | GTGGCAGGACACTGCAGAACAGCTTCATGTTCCCAAAATCACCGTACGACTCTTTTCC |
| | AAACACAGGCAAATCGGAAATCAGCAGGACGACTGTTTTCCCAACACGGGTAAATCTA |
| | GTACCATGTCGTAGTTACTCTCAGGCATGGATGAATAAATGTTATAGCTGC |

| | |
|---|---|
| ORF Start: ATG at 227 | ORF Stop: TGA at 2891 |

| | | |
|---|---|---|
| SEQ ID NO: 94 | 888 aa | MW at 98085.8 kD |

| | |
|---|---|
| NOV30a, | MSAVCNDLRWNIMRALAVLSVTLVMACTEAFFPFISRGKELLWGKPEESRVSSVLEES |
| CG128489- | |
| 01 Protein | KRLVDTAMYATMQRNLKKRGILSPAQLLSFSKLPEPTSGVIARAAEIMETSIQAMKRK |
| Sequence | |
| | VNLKTQQSQHPTDALSEDLLSIIANMSGCLPYMLPPKCPNTCLANKYRPITGACNNRD |
| | HPRWGASNTALARWLPPVYEDGFSQPRGWNPGFLYNGFPLPPVREVTRHVIQVSNEVV |
| | TDDDRYSDLLMAWGQYIDHDIAFTRQSTSKAAFGGGADCQMTCENQNPCFPIQLPEEA |
| | RPAAGTACLPFYRSSAACGTGDQGALFGNLSTANPRQQMNGLTSFLDASTVYGSSPAL |
| | ERQLRNWTSAEGLLRVHARLRDSGRAYLPFVPPRAPSACAPEPGIPGETRGPCFLAGD |
| | GRASEVPSLTALHTLWLREHNRLAAALKALNAHWSADAVYQEARKVVGALHQIITLRD |
| | YIPRILGPEAFQQYVGPYEGYDSTANPTVSNVFSTAAFRFGHATIHPLVRRLDASFQE |
| | HPDLRGLWLHQAFFSPWTLLRGGYNEWREFCGLPRLETPADLSTAIASRSVADKILDL |
| | YKHPDNIDVWLGGLAENFLPRARTGPLFACLIGKQMKALRDGDWFWWENSHVFTDAQR |
| | RELEKHSLSRVICDNTGLTRVPMDAFQVGKFPEDFESCDSIPGMNLEAWRETFRQDDK |
| | CGFPESVENGDFVHCEESGRRVLVYSCRHGYELQGREQLTCTQEGWDFQPPLCKDVNE |
| | CADGAHPPCHASARCRNTKGGFQCLCADPYELGDDGRTCVDSGRLPRATWISMSLAAL |
| | LIGGFAGLTSTVICRWTRTGTKSTLPISETGGGTPELRCGKHQAVGTSPQRAAAQDSE |
| | QESAGMEGRDTHRLPRAL |

Further analysis of the NOV30a protein yielded the following properties shown in Table 30B.

TABLE 30B
Protein Sequence Properties NOV30a

| | |
|---|---|
| PSort | 0.4600 probability located in plasma membrane: 0.1676 |
| analysis: | probability located in microbody (peroxisome); 0.1000 |
| | probability located in endoplasmic reticulum (membrane); |
| | 0.1000 probability located in endoplasmic reticulum (lumen) |
| SignalP | Cleavage site between residues 31 and 32 |
| analysis: | |

A search of the NOV30a protein against the Geneseq database, a proprietary database that contains sequences published in patents and patent publication, yielded several homologous proteins shown in Table 30C.

TABLE 30C
Geneseq Results for NOV30a

| | | NOV30a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAR75689 | Human thryoid peroxidase-Homo | 13 . . . 888 | 873/933 (93%) | 0.0 |
| | sapiens, 933 aa. [EP655502-A, | 1 . . . 933 | 875/933 (93%) | |
| | 31 May 1995] | | | |
| AAW48781 | Thyroid peroxidase-Homo sapiens, | 13 . . . 888 | 872/933 (93%) | 0.0 |
| | 948 aa. [WO9820354-A2, | 16 . . . 948 | 876/933 (93%) | |
| | 14 May 1998] | | | |
| AAW48782 | Thyroid peroxidase deletion mutant- | 13 . . . 802 | 771/847 (91%) | 0.0 |
| | Homo sapiens, 852 aa. [WO9820354- | 16 . . . 851 | 776/847 (91%) | |
| | A2, 14 MAY 1998] | | | |
| AAW48791 | Thyroid peroxidase deletion mutant 10- | 13 . . . 741 | 704/786 (89%) | 0.0 |
| | Homo sapiens, 881 aa. | 16 . . . 790 | 708/786 (89%) | |
| | [WO9820354-A2, 14 May 1998] | | | |
| AAW48790 | Thyroid peroxidase deletion mutant 9- | 13 . . . 570 | 556/615 (90%) | 0.0 |
| | Homo sapiens, 740 aa. | 16 . . . 630 | 558/615 (90%) | |
| | [WO9820354-A2, 14 May 1998] | | | |

In a BLAST search of public sequence databases, the NOV30a protein was found to have homology to the proteins shown in the BLASTP data in Table 30D.

TABLE 30D
Public BLASTP Results for NOV30a

| | | NOV30a | | |
|---|---|---|---|---|
| Protein | | Residues/ | Identities/ | |
| Accession | | Match | Similarities for | Expect |
| Number | Protein/Organism/Length | Residues | the Matched Portion | Value |
| P07202 | Thyroid peroxidase precursor (EC | 13 . . . 888 | 874/933 (93%) | 0.0 |
| | 1.11.1.8) (TPO)-Homo sapiens | 1 . . . 933 | 876/933 (93%) | |
| | (Human), 933 aa. | | | |
| OPHUIT | iodide peroxidase (EC 1.11.1.8) | 13 . . . 888 | 872/933 (93%) | 0.0 |
| | precursor, thyroid-human, 933 aa. | 1 . . . 933 | 874/933 (93%) | |
| AAA61217 | Thyroid peroxidase-Homo sapiens | 13 . . . 888 | 868/933 (93%) | 0.0 |
| | (Human), 933 aa. | 1 . . . 933 | 871/933 (93%) | |
| P14650 | Thyroid peroxidase precursor (EC | 13 . . . 874 | 633/919 (68%) | 0.0 |
| | 1.11.1.8) (TPO)-Rattus norvegicus | 1 . . . 905 | 718/919 (77%) | |
| | (Rat), 914 aa. | | | |
| P09933 | Thyroid peroxidase precursor (EC | 13 . . . 876 | 620/926 (66%) | 0.0 |
| | 1.11.1.8) (TPO)-Sus scrofa (Pig), | 1 . . . 924 | 703/926 (74%) | |
| | 926 aa. | | | |

PFam analysis predicts that the NOV30a protein contains the domains shown in the Table 30E.

TABLE 30E
Domain Analysis of NOV30a

| | | Identities/ | |
|---|---|---|---|
| Pfam | NOV30a | Similarities for | Expect |
| Domain | Match Region | the Matched Region | Value |
| An_peroxidase | 162 . . . 658 | 208/622 (33%) | 1.5e−122 |
| | | 374/622 (60%) | |
| Sushi | 697 . . . 749 | 18/63 (29%) | 2.5e−06 |
| | | 38/63 (60%) | |
| EGF | 755 . . . 793 | 15/47 (32%) | 1.2e−08 |
| | | 33/47 (70%) | |

### Example 31

The NOV31 clone was analyzed, and the nucleotide and encoded polypeptide sequences are shown in Table 31A.

TABLE 31A

NOV31 Sequence Analysis

| | |
|---|---|
| | SEQ ID NO: 95 2921 bp |
| NOV31a. | GGGCTCCGGAGGCCATGCCGGCGTTGGCGCGCGACGGCGGCCAGCTGCCGCTGCTCGT |
| CG128825-01 | |
| DNA | TGTTTTTTCTGCCATGATATTTGGGACTATTACAAATCAAGATCTGCCTGTGATCAAG |
| Sequence | |
| | TGTGTTTTAATCAATCATAAGAACAATGATTCATCAGTGGGGAAGTCATCATCATATC |
| | CCATGGTATCAGAATCCCCGGAAGACCTCGGGTGTGCGTTGAGACCCCAGAGCTCAGG |
| | GACAGTGTACGAAGCTGCCGCTGTGGAAGTGGATGTATCTGCTTCCATCACACTGCAA |
| | GTGCTGGTCGATGCCCCAGGGAAACATTTCCTGTCTCTGGGTCTTTAAGCACAGCTCCC |
| | TGAATTGCCAGCCACATTTTGATTTACAAAACAGAGGAGTTGTTTCCATGGTCATTTT |
| | GAAAATGACAGAACCCAAAGCTGGAGAATACCTACTTTTTATTCAGAGTGAAGCTACC |
| | AATTACACAATATTGTTTACAGTGAGTATAAGAAATACCCTGCTTTACACATTAAGAA |
| | GACCTTACTTTAGAAAAATGGAAAACCAGGACGCCCTGGTCTGCATATCTGAGAGCGT |
| | TCCAGAGCCGATCGTGGAATGGGTGCTTTGCGAATTCACAAGGGGGAGCTGTAAAGAA |
| | GAAAGTCCAGCTGTTGTTAAAAAGGAGGAAAAAGTGCTTCATGAATTATTTGGGATGG |
| | ACATAAGGTGCTGTGCCAGAATGAACTGGGCAAGGGAATGCACCAGGCTGTTCACAAT |
| | AGATCTAATCAACTCCTCAGACCACATTGCCACCAATTATTTCTTTAAAGTAGGGGAA |
| | CCCTTATGGATAAGGTGCAAAGCTGTTCATGTGCAACCATGATTCGGGCTCACCTGGG |
| | AAATTAGAACAAAAGCACTCGAGGAGGGCAACTACTTTGAGATGAGTACCTATTCAAC |
| | AACAGAACTATGATACGGATTCTGTTTGCTTTTGTATCATCAGTGGCAAGAAAACGAC |
| | ACCGGATACTACACTTGTTCCTCTTCAAAGCATCCCAGTCAATCAGCTTTGGTTACCA |
| | TCGTAGAAAAGGGATTTATAAATGCTACCAATTCAAGTGAAGATTATGAAATTGACCA |
| | ATATGAAGAGTTTTGTTTTTCTGTCAGGTTTAAAAGCCTACCCACAATCAGATGTACG |
| | TGGACCTTCTCTCGAAAATCATTTCCTTGTGAGCAAAAGGGTCTTGATAACGGATACA |
| | GCATATCCAAGTTTTGCAATCATAAGCACCAGCCAGGAGAATATATATTCCATGCAGA |
| | AAATGATGATGCCCAATTTACCAAAATGTTCACGCTGAATATAAGAAGGAAACCTCAA |
| | GTGCTCGCAGAAGCATCGGCAAGTCAGGCGTCCTGTTTCTCGGATGGATACCCATTAC |
| | CATCTTGGACCTGGAAGAAGTGTTCAGACAAGTCTCCCAACTGCACAGAAGAGATCAC |
| | AGAAGGAGTCTGGAATAGAAAGGCTAACAGAAAAGTGTTTGGACAGTGGGTGTCGAGC |
| | AGTACTCTAAACATGAGTGAAGCCATAAAAGGGTTCCTGGTCAAGTGCTGTGCATACA |
| | ATTCCCTTGGCACATCTTGTGAGACGATCCTTTTAAACTCTCCAGGCCCCTTCCCTTT |
| | CATCCAAGACAACATCTCATTCTATGCAACAATTGGTGTTTGTCTCCTCTTCATTGTC |
| | GTTTTAACCCTGCTAATTTGTCACAAGTACAAAAAGCAATTTAGGTATGAAAGCCAGC |
| | TACAGATGGTACAGGTGACCGGCTCCTCAGATAATGAGTACTTCTACGTTGATTTCAG |
| | AGAATATGAATATGATCTCAAATGGGAGTTTCCAAGAGAAAATTTAGAGTTTGGGAAG |
| | GTACTAGGATCAGGTGCTTTTGGAAAAGTGATGAACGCAACAGCTTATGGAATTAGCA |
| | AAACAGGAGTCTCAATCCAGGTTGCCGTCAAAATGCTGAAAGAAAAAGCAGACAGCTC |
| | TGAAAGAGAGGCACTCATGTCAGAACTCAAGGTGATGACCCAGCTGGGAAGCCACGAG |
| | AATATTGTGAACCTGCTGGGCGCGTGCACACTGTCAGGACCAATTTACTTGATTTTTG |
| | AATACTGTTGCTATGGTGATCTTCTCAACTATCTAAGAAGTAAAAGAGAAAAATTTCA |
| | CAGGACTTGAACAGAGATTTTCAAGGAACACAATTTCAGTTTTTACCCCACTTTCCAA |
| | TCACATCCAAATTCCAGCATGCCTGGTTCAAGAGAAGTTCAGATACACCCGGACTCGG |
| | ATCAAATCTCAGGGCTTCATGGGAATTCATTTCACTCTGAAGATGAAATTGAATATGA |
| | AAACCAAAAAAGGCTGGAAGAAGAGGAGGACTTGAATGTGCTTACATTTGAAGATCTT |
| | CTTTGCTTTGCATATCAAGTTGCCAAAGGAATGGAATTTCTGGAATTTAAGTCGGCCC |
| | GTCTGCCTGTAAAATGGATGGCCCCCGAAAGCCTGTTTGAAGGCATCTACACCATTAA |
| | GAGTGATGTCTGGTCATATGGAATATTACTGTGGGAAATCTTCTCACTTGGTGTGAAT |
| | CCTTACCCTGGCATTCCGGTTGATGCTAACTTCTACAAACTGATTCAAAATGGATTTA |
| | AAATGGATCAGCCATTTTATGCTACAGAAGAAATATACATTATAATGCAATCCTGCTG |
| | GGCTTTTGACTCAAGGAAACGGCCATCCTTCCCTAATTTGACTTCGTTTTTAGGATGT |
| | CAGCTGGCAGATGCAGAAGAAGCGATGTATCAGAATGTGGATGGCCGTGTTTCGGAAT |
| | GTCCTCACACCTACCAAAACAGGCGACCTTTCAGCAGAGAGATGGATTTGGGGCTACT |
| | CTCTCCGCAGGCTCAGGTCGAAGATTCGTAGAGGAACAATTTAGTTTTAAGGACTTCA |
| | TCCCTCCACCTATCCCTAACA |
| | ORF Start: ATG at 15 ORF Stop: TAG at 2871 |
| | SEQ ID NO: 96 952 aa MW at 108375.0kD |
| NOV31a. | MPALARDGGQLRLLVVFSANTFGTTTNQDLPVIKCVLINHKNNDSSVGKSSSYPMVSE |
| CG128825-01 | |
| Protein | SPEDLGCALRPQSSGTVYEAAAVEVDVSASITLQVLVDAPGNISCLWVPKHSSLNCQP |
| Sequence | |
| | HFDLQNRGVVSMVILKMTETQAGEYLLFIQSEATNYTILFTVSIRNTLLYTLRRPYFR |
| | KMENQDALVCISESVPEPIVEWVLCDSQGFSCKEESPAVVKKEEKVLHELFGMDIRCC |
| | ARNELGRECTRLFTIDLNQTRQTTLRQLFLKVGEPLWIRCKAVHVNHGFGLTWELENK |
| | ALEEGNYFEMSTYSTNRTMIRILFAFVSSVARNDTGYYTCSSSKHPSQSALVTIVEKG |
| | FINATNSSEDYETDQYEEFCFSVRFKAYPQIRCTWTFSRKSPPCEQKGLDNGYSISKF |
| | CNHKHQPGEYIFHAENDDAQFTKMFTLNIRRKPQVLAEASASQASCFSDGYPLPSWTW |
| | KKCSDKSPNCTEEITEGVWNRKANRKVFGQWVSSSTLHMSEAIKGFLVKCCAYNSLGT |
| | SCETILLNSPGPFPFIQDNISFYATIGVCLLFIVVLTLLICHKYKKQFRYESQLQMVQ |
| | VTGSSDNEYFYVDFREYEYDLKWEFPRENLEFGKVLGSGAPGKVMNATAYGISKTGVS |
| | IQVAVKMLKEKADSSEREALMSELKVMTQLGSHENIVNLLGACTLSGPIYLTPEYCCY |
| | GDLLNYLRSKREKFHRTWTEIFKEHNFSFYRTFQSHPNSSMPGSREVQTHPDSDQISG |
| | LHGNSFHSEDEIEYENQKRLEEEEDLNVLTFEDLLCFAYQVAKGMEFLEFKSARLPVK |
| | WMAPESLFEGIYTIKSDVWSYGILLWEIFSLGVNPYPGIPVDANFYKLIQNGFKMDQP |
| | FYATEETYIIMQSCWAFDSRKRPSFPNLTSFLGCQLADAEEAMYQNVDGRVSECPHTY |
| | QNRRPFSREMDLGLLSPQAQVEDS |
| | SEQ ID NO: 97 3270 bp |
| NOV31b, | ATGCCGGCGTTGGCGCGCGACGGCGGCCAGCTGCCGCTGCTCGTTGTTTTTTCTGCAA |
| CG128825-02 | |
| DNA | TGATATTTGGGACTATTACAAATCAGATCTGCCTGTGATCAAAGTGTGTTTTAATCAA |
| Sequence | |
| | TCATAAGAACAATGATTCATCAGTGGGGAAGTCATCATCATATCCCATGGTATCAGAA |
| | TCCCCGGAAGACCTCGGGTGTGCGTTGAGACCCCAGAGCTCAGGGACAGTGTACGAAC |
| | GTGCCGCTGTGGAAGTGGATGTATCTGCTTCCATCACACTGCAAGTGCTGGTCGATGC |
| | CCCAGGGAACATTTCCTGTCTCTGGGTCTTTAAGCACAGCTCCCTGAATTGCCAGCCA |
| | CATTTTGATTTACAAAACAGAAGGAGTTGTTTCCATGGTCATTTTGGAATGACAGAAA |
| | CCCAAGCTGGAGAATACCTACTTTTTATTCAGAGTGAAGCTACCAATTACACAATATT |
| | GTTTACAGTGAAGTATAAGAATACCCTGCTTTACACATTAAGAAGACCTTACTTTAGA |
| | AAAATGGAAAACCAGGACGCCCTGGTCTGCATATCTGAGAGCGTTCCAGAGCCGATCG |
| | TGGAATGGGTGCTTTGCGATTCACAGGGGGAAAGCTGTAAAGAAGAAAGTCCAGCTCT |
| | TGTTAAAAAGGAGGAAAAGTGCTTCATGAATTATTTGGGATGGACATAAGGTGCTGT |
| | GCCAGAATGAACTGGGCAGGGAATGCACCAGGCTGTTCACAATAGATCTAAATCAAA |
| | CTCCTCAGACCACATTGCCACAATTATTTCTTAAGTAGGGGAACCCTTATGGATAAG |
| | GTGCAAAGCTGTTCATGTGAACCATGGATTCGGGCTCACCTGGGAATTAGAAAACAA |
| | GCACTCGAGGAGGGCAACTACTTTGAGATGAGTACCTATTCAACAACAGAACTATGA |
| | TACGGATTCTGTTTGCTTTTGTATCATCAGTGGCAAGAACGACACCGGATACTACAC |
| | TTGTTCCTCTTCAAGCATCCCAGTCAATCAGCTTTGGTTACCATCGTAGAAAAGGGA |
| | TTTATAATGCTACCAATTCAAGTGAAGATTATGAAATTGACCAATATGAAGAGTTTT |
| | GTTTTTCTGTCAGGTTTAAAGCCTACCCACAATCAGATGTACGTGGACCTTCTCTCG |
| | AAAATCATTTCCTTGTGAGCAAAGGGTCTTGATAACGGATACAGCATATCCAAGTTT |
| | TGCAATCATAAGCACCAGCCAGGAGAATATATATTCCATGCAGAAATGATGATGCCC |
| | AATTTACCAAAATGTTCACGCTGATATAAGAAGGAAACCTCAAGTGCTCGCAGAAGC |
| | ATCGGCAAGTCAGGCGTCCTGTTTCTCGGATGGATACCCATTACCATCTTGGACCTGG |
| | AAGAAGTGTTCAGACAAGTCTCCCAACTGCACAGAAGAGATCACAGAAGGAGTCTGGA |
| | ATAGAAAGGCTAACAGAAAAGTGTTTGGACAGTGGGTGTCGAGCAGTACTCTAAACAT |
| | GAGTGAAGCCATAAAAGGGTTCCTGGTCAAGTGCTGTGCATACAATTCCCTTGGCACA |
| | TCTTGTGAGACGATCCTTTTAACTCTCCAGGCCCCTTCCCTTTCATCCAAAGACAACA |
| | TCTCATTCTATGCAACAATTGGTGTTTGTCTCCTCTTCATTGTCGTTTTAACCCTGCT |
| | AATTTGTCACAAGTACAAAAAGCAATTTAGGTATGAAGCCAAGCTACAGATGGTACAG |
| | GTGACCGGCTCCTCAGATAATGAGTACTTCTACGTTGATTTCAGAGAATATGAATATG |
| | ATCTCAAATGGGAGTTTCCAAGAGAAAATTTAGAGTTTGGGAAGGTACTAGGATCAGG |
| | TGCTTTTGGAAAGTGATGAACGCAACAGCTTATGGAAATTAGCAAAACAGGAGTCTCA |
| | ATCCAGGTTGCCGTCAAAATGCTGAAAGAAAAGCAGACAGCCTCTGAAAGAGAGGCAC |
| | TCATGTCAGAACTCAAGATGATGACCCAGCTGGGAAGCCACGAGAATATTGTGAACCT |
| | GCTGGGGGCGTGCACACTGTCAGGACCAATTTACTTGATTTTTGAATACTGTTGCTAT |
| | GGTGATCTTCTCAACTATCTAAGAAGTAAAAGAGAAAAATTTCACAGGACTTGGACAG |
| | AGATTTTCAAGGAACACAATTTCAGTTTTTACCCCACTTTCCAATCACATCCAAATTC |
| | CAGCATGCCTGGTTCAAGAGAAGTTCAGATACACCCGGACTCGGATCAAATCTCAGGG |
| | CTTCATGGGAATTCATTTCACTCTGAAGATGAAATTGAATATGAAAACCAAAAAAGGC |
| | TGGAAGAAGAGGAGGACTTGAATGTGCTTACATTTGAAGATCTTCTTTGCTTTGCATA |
| | TCAAGTTGCCAAAGGAATGGAATTTCTGGAATTTAAGTCGTGTGTTCACAGAGACCTG |
| | GCCGCCAGGAACGTGCTTGTCACCCACGGGAAAGTGGTGAAGATATGTGACTTTGGAT |
| | TGGCTCGAGATATCATGAGTGATTCCAACTATGTTGTCAGGGGCAATGCCCGTCTGCC |
| | TGTAAAATGGATGGCCCCCGAAAGCCTGTTTGAAGGCATCTACACCATTAAGAGTGAT |
| | GTCTGGTCATATGGAATATTACTGTGGGAAATCTTCTCACTTGGTGTGAATCCTTACC |
| | CTGGCATTCCGGTTGATGCTAACTTCTACAAACTGATTCAAAATGGATTTAAAATGGA |
| | TCAGCCATTTTATGCTACAGAGAAATATACATTATAAATGCAATCCTGCTGGGCTTTT |
| | GACTCAAGGAAACGGCCATCCTTCCCTAATTTGACTTCGTTTTTAGGATGTCAGCTGG |
| | CAGATGCAGAAGAAGCGAAACTGTGGAAAATCCCTGAGACAATGAAAGCAGTTAAAAT |
| | TGCACCGCAGAGGGAAAACCCACCACAGAGGATGCCTGGGAAAAACAAGGACAAGGGT |
| | AACACAAAGGCAGCAAGAAGTCCTGGGACACTGCAGAAGTTCTGAAGCAGGAGCAGCC |
| | ACATGGTGAAATCAACATAAGATTAAATATGTATCAGAATGTGGATGGCCGTGTTTCG |
| | GAATGTCCTCACACCTACCAAAACAGGCGACCTTTCAGCAGAGAGATGGATTTGGGGC |
| | TACTCTCTCCGCAGGCTCAGGTCGAAGATTCGTAGAGGAACAATTTAGTTTTAAGGAC |
| | TTCATCCCTCCACCTATCCCTAACAGGCTGTAGATTACCAAAACAAGATTAATTTCAT |
| | CACTAAAAGAAAATCTATTATC |
| | ORF Start: ATG at 1 ORF Stop: TGA at 3001 |
| | SEQ ID NO: 98 1000 aa MW at 113678.6kD |
| NOV31b, | MPALARDGGQLPLLVVFSAMIFGTTTNQDLPVIKCVLINHKNNDSSVGKSSSYPMVSE |
| CG128825-02 | |
| Protein | SPEDLGCALRPQSSGTVYERAAVEVDVSASITLQVLVDAPGNISCLWVFKHSSLNCQP |
| Sequence | |
| | HFDLQNRGVVSMVILKMTETQAGEYLLFTQSEATNYTTLFTVSIRNTLLYTLRRRYFR |
| | KMENQDALVCISESVPEPIVEWVLCDSQGESCKEESPAVVKKEEKVLHELFGMDIRCC |
| | ARNELGRECTRLFTIDLNQTPQTTLPQLFLKVGEPLWIRCKAVHVNHGFGLTWELENK |
| | ALEEGNYFEMSTYSTNRTMIRTLFAFVSSVARNDTGYYTCSSSKHPSQSALVTIVEKG |
| | FINATNSSEDYEIDQYEEFCFSVRFKAYPQIRCTWTFSRKSFPCEQKGLDNGYSISKE |
| | CNHKHQPGEYTFHAENDDAQFTKMFTLNIRRKPQVLAEASASQASCPSDGYPLPSWTW |
| | KKCSDKSPNCTEEITEGVWNRKANRKVFGQWVSSSTLNMSEATKGFLVKCCAYNSLGT |
| | SCETILLNSPGPFPFIQDNISFYATIGVCLLFIVVLTLLICHKYKKQFRYESQLQMVQ |
| | VTGSSDNEYPYVDFREYEYDLKWEFPRENLEFGKVLGSGAFGKVMNATAYGISKTGVS |
| | IQVAVKMLKEKADSSEREALMSELKMMTQLGSHENIVNLLGACTLSGPIYLIFEYCCY |
| | GDLLNYLRSKREKFHRTWTEIFKEHNFSFYPTFQSHPNSSMRGSREVQTHPDSDQISG |
| | LHGNSFHSEDEIEYENQKRLEEEEDLNVLTFEDLLCFAYQVAKGMEFLEFKSCVHRDL |
| | AARNVLVTHGKVVKICDFGLARDIMSDSNYVVRGNARLPVKWMAPESLFEGIYTIKSD |
| | VWSYGILLWEIFSLGVNPYPGIRVDANFYKLIQNGFKMDQRFYATEEIYIIMQSCWAF |
| | DSRKRRSFPNLTSFLGCQLADAEEAKLWKIPETMKAVKIAPQRENPPQRMPGKNKDKG |
| | NTKAARSPGTLQKF |

Sequence comprising of the above protein sequences yields the following sequences relationships shown in Table 31B.

TABLE 31B
Comparison of NOV31a against NOV31b

| | | Identities/ |
|---|---|---|
| Protein | NOV31a Residues/ | Similarities for |
| Sequence | Match Residues | the Matched Region |
| NOV31b | 1 . . . 912 | 910/953 (95%) |
| | 1 . . . 953 | 911/953 (95%) |

Further analysis of the NOV31a protein yielded the following properties shown in Table 31C.

TABLE 31C
Protein Sequence Properties NOV31a

| | |
|---|---|
| PSort | 0.4600 probability located in plasma membrane; 0.1662 |
| analysis: | probability located in microbody (peroxisome); 0.1000 |
| | probability located in endoplasmic reticulum (membrane); |
| | 0.1000 probability located in endoplasmic reticulum (lumen) |
| SignalP | Cleavage site between residues 28 and 29 |
| analysis: | |

A search of the NOV31a protein against the Geneseq database, a proprietary database that contains sequences published in patents and patent publication, yielded several homologous proteins shown in Table 31D.

TABLE 31D
Geneseq Results for NOV31a

| | | NOV31a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAR75961 | Human STK-1-Homo sapiens, 993 | 1 . . . 952 | 947/993 (95%) | 0.0 |
| | aa. [WO9519175-A, 20 Jul. 1995] | 1 . . . 993 | 949/993 (95%) | |
| AAY08617 | Human flk-2 protein-Homo sapiens, | 1 . . . 952 | 946/993 (95%) | 0.0 |
| | 993 aa. [U.S. Pat. No. 5912133-A, | 1 . . . 993 | 948/993 (95%) | |
| | 15 Jun. 1999] | | | |
| AAW19873 | Human flk-2 receptor-Homo | 1 . . . 952 | 946/993 (95%) | 0.0 |
| | sapiens, 993 aa. [U.S. Pat. No. 5621090-A, | 1 . . . 993 | 948/993 (95%) | |
| | 15 Apr. 1997] | | | |
| AAR97419 | Murine foetal liver kinase 2-Mus | 1 . . . 952 | 946/993 (95%) | 0.0 |
| | musculus, 993 aa. [U.S. Pat. No. 5548065-A, | 1 . . . 993 | 948/993 (95%) | |
| | 20 Aug. 1996] | | | |
| AAR67536 | Human flk-2-Homo sapiens, 993 aa | 1 . . . 952 | 946/993 (95%) | 0.0 |
| | [U.S. Pat. No. 5367057-A, 22 Nov. 1994] | 1 . . . 993 | 948/993 (95%) | |

In a BLAST search of public sequence databases, the NOV31a protein was found to have homology to the proteins shown in the BLASTP data in Table 31E.

TABLE 31E
Public BLASTP Results for NOV31a

| | | NOV31a | | |
|---|---|---|---|---|
| Protein | | Residues/ | Identities/ | |
| Accession | | Match | Similarities for | Expect |
| Number | Protein/Organism/Length | Residues | the Matched Portion | Value |
| P36888 | FL cytokine receptor precursor (EC | 1 . . . 952 | 946/993 (95%) | 0.0 |
| | 2.7.1.112) (Tyrosine-protein kinase | 1 . . . 993 | 948/993 (95%) | |
| | receptor FLT3) (Stem cell tyrosine kinase | | | |
| | 1) (STK-1) (CD135 antigen)-Homo | | | |
| | sapiens (Human), 993 aa. | | | |
| A36873 | protein-tyrosine kinase (EC 2.7.1.112) | 1 . . . 952 | 946/994 (95%) | 0.0 |
| | STK-1 precursor-human, 993 aa. | 1 . . . 993 | 948/994 (95%) | |
| S18827 | Flt3 protein-mouse, 1000 aa. | 1 . . . 950 | 812/994 (81%) | 0.0 |
| | | 1 . . . 994 | 867/994 (86%) | |
| Q00342 | FL cytokine receptor precursor (EC | 1 . . . 912 | 788/956 (82%) | 0.0 |
| | 2.7.1.112) (Tyrosine-protein kinase | 1 . . . 956 | 841/956 (87%) | |
| | receptor flk-2) (Fetal liver kinase 2) | | | |
| | musculus (Mouse), 992 aa. | | | |
| O97745 | Mast/stem cell growth factor receptor-Sus | 47 . . . 917 | 282/911 (30%) | e−103 |
| | scrofa (Pig), 923 aa (fragment). | 20 . . . 894 | 437/911 (47%) | |

PFam analysis predicts that the NOV31a protein contains the domains shown in the Table 31F.

TABLE 31F
Domain Analysis of NOV31a

| | | Identities/ | |
|---|---|---|---|
| Pfam | NOV31a | Similarities for | Expect |
| Domain | Match Region | the Matched Region | Value |
| Ig | 265 . . . 332 | 14/70 (20%) | 2.1e−06 |
| | | 45/70 (64%) | |
| Pkinase | 610 . . . 710 | 35/102 (34%) | 2.7e−24 |
| | | 84/102 (82%) | |
| Pkinase | 782 . . . 803 | 6/22 (27%) | 0.98 |
| | | 22/22 (100%) | |
| Pkinase | 810 . . . 898 | 26/124 (21%) | 4.2e−18 |
| | | 66/124 (53%) | |

### Example 32

The NOV32 clone was analyzed and the nucleotide and encoded poly,peptide sequences are shown in Table 32A.

TABLE 32A
NOV32 Sequence Analysis+HZ,1/46

| | |
|---|---|
| | SEQ ID NO 99 5347 bp |
| NOV32a. | AAATCGAAGCAAACATGTCTGGAGAAGTGCGTTTGAGGCAGTTGGAGCAGTTTATTTT |
| CG128891-01 | |
| DNA | GGACGGGCCCGCTCAGACCAATGGGCAGTGCTTCAGTGTGGAGACATTACTGGATATA |
| Sequence | |
| | CTCATCTGCCTTTATGATGAATGCAATAATTCTCCATTGAGAAGAGAGAAGAACATTC |
| | TCGAATACCTAGAATGGGGTGCTAAACCATTTACTTCTAAAGTGAAACAAATGCGATT |
| | ACATAGAGAAGACTTTGAAATATTAAAGGTGATTGGTCGAGGAGCTTTTGGGGAGGTT |
| | GCTGTAGTAAAACTAAAAAATGCAGATAAAGTGTTTGCCATGAAAATATTGAATAAAT |
| | GGGAAATGCTGAAAAGAGCTGAGACAGCATGTTTTCGTGAAGAAAGGGATGTATTAGT |
| | GAATGGAGACAATAAATGGATTACAACCTTGCACTATGCTTTCCAGGATGACAATAAC |
| | TTATACCTGGTTATGGATTATTATGTTGGTGGGGATTTGCTTACTCTACTCAGCAAAT |
| | TTGAAGATAGATTGCCTGAAGATATGGCTAGATTTTACTTGGCTGAGATGGTGATAGC |
| | AATTGACTCAGTTCATCAGCTACATTATGTACACAGAGACATTAAACCTGACAATATA |
| | CTGATGGATATGAATGGACATATTCGGTTAGCAGATTTTGGTTCTTGTCTGAAGCTGA |
| | TGGAAGATGGAACGGTTCAGTCCTCAGTGGCTGTAGGAACTCCAGATTATATCTCTCC |
| | TGGTCTTTGGGGGTCTGTATGTATGAAATGCTTTACGGAGAAACACCATTTTATGCAG |
| | AATCGCTGGTGGAGACATACGGAAAAATCATGAACCACAAAGAGAGGTTTCAGTTTCC |
| | AGCCCAAGTGACTGATGTGTCTGAAAATGCTAAGGATCTTATTCGAAGGCTCATTTGT |
| | AGCAGAGAACATCGACTTGGTCAAATGGAATAGAAGACTTTAAGAAACACCCATTTT |
| | TCAGTGGAATTGATTGGGATAATATTCGGAACTGTGAAGCACCTTATATTCCAGAAGT |
| | TAGTAGCCCAACAGATACATCGAATTTTGATGTAGATGATGATTGTTTAAAAAATTCT |
| | GAAACGATGCCCCCACCAACACATACTGCATTTTCTGGCCACCATCTGCCATTTGTTG |
| | GTTTTACATATACTAGTAGCTGTGTACTTTCTGATCGGAGCTGTTTAAGAGTTACGGC |
| | TGGTCCCACCTCACTGGATCTTGATGTTAATGTTCAGAGGACTCTAGACAACAACTTA |
| | GCAACTGAAGCTTATGAAAGAAGAATTAAGCGCCTTGAGCAAGAAAAACTTGAACTCA |
| | GTAGAAAACTTCAAGAGTCAACACAGACTGTCCAAGCTCTGCAGTATTCAACTGTTGA |
| | TGGTCCACTAACAGCAAGCAAAGATTTAGAAATAAAACTTAAAAGAAGAAAAATTGAA |
| | AAACTAAGAAAACAAGTAACAGAATCAAGTCATTTGGAACAGCAACTTGAAGAAGCTA |
| | ATGCTGTGAGGCAAGAACTAGATGATGCTTTTAGACAAATCAAGGCTTATGAAAAACA |
| | AATCAAAACGTTACAACAAGAAAGAGAAGATCTAAATAAGGAACTAGTCCAGGCTAGT |
| | GAGCGATTAAAAAACCAATCCAAAGAGCTGAAAGACGCACACTGTCAGAGGAAACTGG |
| | CCATGCAGGAATTCATGGAGATCAATGAGCGGCTAACAGAATTGCACACCCAAAAACA |
| | GAAACTTGCTCGCCATGTCCGAGATAAGGAAGAAGAGGTGGACCTGGTGATGCAAAAA |
| | GTTGAAAGCTTAAGGCAAGAACTGCGCAGAACAGAAAGAGCCAAAAAAGAGCTGGAAG |
| | TTCATACAGAAGCTCTAGCTGCTGAAGCATCTAAGACAGGAAAGCTACGTGACAAGAG |
| | TGAGCACTAATTCTAAGCAACTGGAAAATGAATTGGAGGGACTGAAAAAGCACAAATT |
| | AGTTACTCACCAGGAGTATGCAGCATAGAACATCAGCAAAGAGATAACCAAACTAAGA |
| | CTGATTTGGAAAAGAAAAGTATCTTTTATGAAGAAGAATTATCTAAAAGAGAAGGAAT |
| | ACATGCAAATGAAAATAAAAAATCTTAAGAAGAACTGCATGATTCAGAAGGTCAGCAA |
| | CTTGCTCTCAACAAAGAAATTATGATTTTAAAAGACAAATTGGAAAAAACCAGAAGAG |
| | AAAGTCAAAGTGAAAGGGAGGAATTTGAAAGTGAGTTCAAACAACAATATGAACGAGA |
| | AAAAGTGTTGTTAACTGAAGAAAATAAAAAGCTGACGAGTGAACTTGATAAGCTTACT |
| | ACTTTGTATGAGAACTTAAGTATACACAACCAGCAGTTAGAAGAAGAGGTTAAAGATC |
| | TAGCAGACAAGAAGAATCAGTTGCACATTGGGAAGCCCAAATCACAGAAATAAATTCA |
| | GTGGGTCAGCGATGAAAAGGATGCACGATGGTATCTTCAGGCCTTAGCTTCTAAAATG |
| | ACTGAAGAATTGGAGGCATTAAGAAATTCCAGCTTGGGTACACGAGCAACAGTAAGCT |
| | TCTATGATATGCCCTGGAAAATGCGTCGTTTTGCGAAACTGGATATGTCAGCTAGACT |
| | GGAGTTGCAGTCGGCTCTGGATGCAGAATAAAAGAGCCAAACAGGCCATCCAAGAGAG |
| | TTGAATAAAGTTAAAGCATCTAATATCATAACAGAAAAACTAAAAGATTCAGAGAAGA |
| | AGAACTTGGAACTACTCTCAGAAATCGAACAGCTGATAAAGGACACTGAAGAGCTTAG |
| | ATCTGAAAAGGGTATGGAGCACCAAGACTCACAGCATTCTTTCTTGGCATTTTTGAAT |
| | ACGCCTACCGATGCTCTGGATCAATTTGAATCTCCATCCTGTACTCCAGCTAGCAAAG |
| | GCAGACGTGTAAGAGACTCCACTCCACTTTCAGTTCACACACCAACCTTAAGGAAAAA |
| | AGGATGTCCTGGTTCAACTGGCTTTCCACCTAAGCGCAAGACTCACCAGTTTTTTGTA |
| | AAATCTTTTACTACTCCTACCAAGTGTCATCAGTGTACCTCCTTGATGGTGGGTTTAA |
| | TAAGACAAGGGCTGTTCATGTGAAGTGTGTGGATTCTCATGCCATATAACTTGTGTAA |
| | CAAAGCTCCAACCACTTGTCCAGTTCCTCCTGAACAGACAAAAGGTCCCCTGGGTATA |
| | GATCCTCAGAAAGGAATAGGAACAGCATATGAAGGTCATGTCAGGATTCCTAAGCCAG |
| | CTGGAGTGAAGAAAGGGTGGCAGAGAGCACTGGCTATAGTGTGTGACTTCAAACTCTT |
| | TCTGTACGATATTGCTGAAGGAAAAGCATCTCAGCCCAGTGTTGTCATTAGTCAAGTG |
| | ATTGACATGAGGAGGGATGAAGAATTTTCTGTGAGTTCAGTCTTGGCTTCTGATGTTA |
| | TCCATGCAAGTCGGAAAGATATACCCTGTATATTTAGGGTCACAGCTTCCCAGCTCTC |
| | AGCATCTAATAACAAATGTTCAATCCTGATGCTAGCAGACACTGAGAATGAGAAGAAT |
| | AAGTGGGTGGGAGTGCTGAGTGAATTGCACAAGATTTTGAAGAAAAACAAATTCAGAG |
| | ACCGCTCAGTCTATGTTCCCAAGAGGCTTATGACAGCACTCTAACCCCTCATTAAAAC |
| | AACCCAGGCAGCCGCAATCATAGAATCATGAAGAATTGCTTTGGGAAACGAAGAAGGG |
| | TCGTCATGTACGACTTTTTCCTATGTCAGCATTGGATGGGCGAGAGACCGATTTTTAC |
| | AAGCTGTCAGAAACTAAAGGGTGTCAAACCGTAACTTCTGGAAAGGTGCGCCATGGAG |
| | GAGCAAGACCCGTCACAGAAAATTTAAAGAAATTCAAGTCCCATATAATGTCCAGTGG |
| | ATTTATTGCACATCAACCAATGGATGCTATCTGCGCAGTTGAGATCTCCAGTAAAGAA |
| | TATCTGCTGTGTTTTAACAGCATTGGGATATACACTGACTGCCAGGGCCGAAGATCTA |
| | GACAACAGGAATTGATGTGGCCAGCAAATCCTTCCTCTTGTTGTAAGATTCTCTACAA |
| | TGCACCATATCTCTCGGTGTACAGTGPAAATGCAGTTGATATCTTTGATGTGAAACTCC |
| | ATGGAATGGATTCAGACTCTTCCTCTCAAAAAGGTACGACCCTTAAAACAATGAAGGAT |
| | CATTAAATCTTTTAGGGTTGGAGACCATTAGAATTAATATATTTCAAAAATAAGATGGC |
| | AGAAGGGGACGAACTGGTAGTACCTGTTACATCAGATAATAGTCGGAAAACAAATGGTT |
| | AGAAACATTAACAATAAGCGGCGTTATTCCTTCAGAGTCCCAGAAGAGGAAAGGATGC |
| | AGCAGAGGAGGGAAATGCTACGAGATCCAGAATGAGAAATAAATTAAATTTCTAATCC |
| | AACTAATTTTAATCACATAGCACACATGGGTCCTGGAGATGGAATACAGATCCTGAAA |
| | GATCTGCCCATGAACCCTCGGCCTCAGGAAAGTCGGACAGTATTCAGTGGCTCAGTCA |
| | GTATTCCATCTATCACCAAATCCCGCCCTGAGCCAGGCCGCTCCATGAGTGCTAGCAG |
| | TGGCTTGTCAGCATCATCCGCACAGAATGGCAGCGCATTAAAAGAGGGATTCTCTGGA |
| | GGAAGCTACAGTGCCAAGCGGCAGCCCATGCCCTCCCCGTCAGAGGGCTCTTTGTCCT |
| | CTGGAGGCATGGACCAAGGAAGTGATGCCCCAGCGAGGGACTTTGACGGAGAGGACTC |
| | TGACTCTCCGAGGCATTCCACAGCTTCCACAGTTCCAACCTAAGCAAGCCCCCCAAGC |
| | CCAGCTTCACCCCGAAAAACCAAGAGCCTCTCCCTGGAGAGCACTGACCGCGGGAGCT |
| | GGGACCCGTGAGCTGCCTCAGCACTGGGACCTCTCGCTCTCCGCTCCCTGCCACTCGC |
| | CTCCTCTCACTTTCATCTCTTCCCTCCACCTCGCCTGCTCGGCCTGAAAGCCACCAGG |
| | GGCTGGCAGCA |
| | ORF Start: ATG at 15 ORF Stop: GA at 5229 |
| | SEQ ID NO: 100 1738 aa MW at 198155.8kD |
| NOV32a. | MSGEVRLRQLEQFILDGPAQTNGQCFSVETLLDILTCLYDECNNSPLRREKNTLEYLE |
| CG18891-01 | |
| Protein | WGAKPFTSKVKQMRLHREDFEILKVIGRGAFGEVAVVKLKNADKVFAMKILNKWEMLK |
| Sequence | |
| | RAETACFREERDVLVNGDNKWITTLHYAFQDDNNLYLVMDYYVGGDLLTLLSKEEDRL |
| | PEDMARFYLAEMVIATDSVHQLHYVHRDIKPDNILMDMNGHIRLADFGSCLKLMEDGT |
| | VQSSVAVGTPDYISPEILQAMEDGKGRYGPFCDWWSLGVCMYEMLYGETRFYAESLVE |
| | TYGKTMNHKERFQFPAQVTDVSENAKDLIRRLICSRFHRLGQNGIEDFKKHPFFSGID |
| | WDNIRNCEAPYIPEVSSRTDTSNFDVDDDCLKNSETMPPPTHTAFSGHHLPFVGFTYT |
| | SSCVLSDRSCLRVTAGPTSLDLDVNVQRTLDNNLATEAYERRIKRLEQEKLELSRKLQ |
| | ESTQTVQALQYSTVDGPLTASKDLEIKNLKEEIEKLRKQVTESSHLEQQLEFANAVRQ |
| | ELDDAFRQIKAYFKQIKTLQQEREDLNKELVQASERLKNQSKELKDAHCQRKLAMQEF |
| | MEINERLTELHTQKQKLARHVRDKEEEVDLVMQKVESLRQELRRTERAKKELEVHTEA |
| | LAAEASKDRKLREQSEHYSKQLENELEGLKQKQISYSPGVCSIEHQQEITKLKTDLEK |
| | KSIFYEEELSKREGIHANEIKNLKKELHDSEGQQLALNKEIMILKDKLEKTRRESQSE |
| | REEFESEFKQQYEREKVLLTEENKKLTSELDKLTTLYENLSIHNQQLEEEVKDLADKK |
| | ESVAHWEAQITEITQWVSDEKDARWYLQALASKMTEELEALRNSSLGTRATVSFYDMP |
| | WKMRRFAKLDMSARLELQSALDAEIRAKQAIQEELNKVKASNIITEKLKDSEKKNLEL |
| | LSETEQLTKDTEELRSEKGMEHQDSQHSFLAFLNTRTDALDQFESPSCTPASKGRRVR |
| | DSTPLSVHTPTLRKKGCPGSTGFPPKRKTHQFFVKSFTTPTKCHQCTSLMVGLTRQGC |
| | SCEVCGFSCHITCVNKAPTTCPVPPEQTKGPLGIDPQKGIGTAYEGHVRIPKPAGVKK |
| | GWQRALAIVCDFKLFLYDIAEGKASQPSVVTSQVIDMRRDEEFSVSSVLASDVIHASR |
| | KDIPCIFRVTASQLSASNNKCSILMLADTENEKNKWVGVLSELHKTLKKNKFRDRSVY |
| | VPKEAYDSTLRLIKTTQAAAIIDHERTALGNEEGLFVVHVTKDEIIRVGDNKKIHQIE |
| | LIPNDQLVAVISGRNRHVRLFPMSALDGRETDFYKLSETKGCQTVTSGKVRHGALTCL |
| | CVAMKRQVLCYELFQSKTRHRKFKEIQVPYNVQWMATFSEQLCVGFQSGFLRYPLNGE |
| | GNPYSMLHSNDHTLSFTAHQPMDAICAVEISSKEYLLCFNSIGIYTDCQGRRSRQQEL |
| | MWPANPSSCCKILYNAPYLSVYSENAVDIFDVNSMEWIQTLPLKKVRPLNNEGSLNLL |
| | GLETTRLIYFKNKMAEGDELVVPETSDNSRKQMVRNINNKRRYSFRVPEEERMQQRRE |
| | MLRDPEMRNKLISNPTNPNHIAHMGPGDGIQILKDLPMNPRPQESRTVFSGSVSTPSI |
| | TKSRPEPGRSMSASSGLSASSAQNGSALKREFSGGSYSAKRQPMPSPSEGSLSSGGMD |
| | QGSDAPARDFDGEDSDSPRHSTASNSSNLSSPPSPASPRKTKSLSLESTDRGSWDP |
| | SEQ ID NO: 101 5875 bp |
| NOV32b, | AAATCGAAGCAAACATGTCTGGAGAAGTGCGTTTGAGGCAGTTGGAGCAGTTTATTTT |
| CG128891-02 | |
| DNA | GGACGGGCCCGCTCAGACCAATGGGCAGTGCTTCAGTGTGGAGACATTACTGGATATA |
| Sequence | |
| | CTCATCTGCCTTTATGATGAATGCAATAATTCTCCATTGAGAAGAGAGAAGAACATTC |
| | TCGAATACCTAGAATGGGGTGCTAAACCATTTACTTCTAkAGTGAAACAAATGCGATT |
| | ACATAGAGAAGACTTTGAAATATTAAAGGTGATTGGTCGAGGAGCTTTTGGGGAGGTT |
| | GCTGTAGTAAAACTAAAAAATGCAGATAAAGTGTTTGCCATGAAAATATTGAATAAAT |
| | GGGAAATGCTGAAAAGAGCTGAGACAGCATGTTTTCGTGAAGAAAGGGATGTATTAGT |
| | GAATGGAGACAATAAATGGATTACAACCTTGCACTATGCTTTCCAGGATGACAATAAC |
| | TTATACCTGGTTATGGATTATTATGTTGGTGGGGATTTGCTTACTCTACTCAGCAAAT |
| | TTGAAGATAGATTGCCTGAAGATATGGCTAGATTTTACTTGGCTGAGATGGTGATAGC |
| | AATTGACTCAGTTCATCAGCTACATTATGTACACAGAGACATTAAACCTGACAATATA |
| | CTGATGGATATGAATGGACATATTCGGTTAGCAGATTTTGGTTCTTGTCTGAAGCTGA |
| | TGGAAGATGGAACGGTTCAGTCCTCAGTGGCTGTAGGAACTCCAGATTATATCTCTCC |
| | TGAAATCCTTCAAGCCATGGAAGATGGAAAAGGGAGATATGGACCTGAATGTGACTGG |
| | TGGTCTTTCGGGGTCTGTATGTATGAAATGCTTTACGGAGAAACACCATTTTATGCAG |
| | AATCGCTGGTGGAGACATACGGAAAAATCATGAACCACAAAGAGAGGTTTCAGTTTCC |
| | AGCCCAAGTGACTGATGTGTCTGAAAATGCTAAGGATCTTATTCGAAGGCTCATTTGT |
| | AGCAGAGAACATCGACTTGGTCAAAATGGAATAGAAGACTTTAAGAAACACCCATTTT |
| | TCAGTGGAATTGATTGGGATAATATTCGGAACTGTGAAGCACCTTATATTCCAGAAGT |
| | TAGTAGCCCAACAGATACATCGAATTTTGATGTAGATGATGATTGTTTAAAAAATTCT |
| | GAAACGATGCCCCCACCAACACATACTGCATTTTCTGGCCACCATCTGCCATTTGTTG |
| | GTTTTACATATACTAGTAGCTGTGTACTTTCTGATCGGAGCTGTTTAAGAGTTACGGC |
| | TGGTCCCACCTCACTGGATCTTGATGTTAATGTTCAGAGGACTCTAGACAACAACTTA |
| | GGAACTGAAGCTTATGAAAGAAGAATTAAGCGCCTTGAGCAAGAAAAACTTGAACTCA |
| | GTAGAAAACTTCAAGAGTCAACACAGACTGTCCAAGCTCTGCAGTATTCAACTGTTGA |
| | TGGTCCACTAACAGCAAGCAAAGATTTAGAAATAAAAAACTTAAAAGAAGAAATTGAA |
| | AAACTAAGAAAACAAGTAACAGAATCAAGTCATTTGGAACAGCAACTTGAAGAAGCTA |
| | ATGCTGTGAGGCAAGAACTAGATGATGCTTTTAGACAAATCAAGGCTTATGAAAAACA |
| | AATCAAAACGTTACAACAAGAAAGAGAAGATCTAAATAAGGAACTAGTCCAGGCTAGT |
| | GAGCGATTAAAAAACCAATCCAAAGAGCTGAAAGACGCACACTGTCAGAGGPAACTGG |
| | CCATGCAGGAATTCATGGAGATCAATGAGCGGCTAACAGAATTGCACACCCAAAAACA |
| | GAAACTTGCTCGCCATGTCCGAGATAAGGAAGAAGAGGTGGACCTGGTGATGCAAAAA |
| | GTTGAAAGCTTAAGGCAAGAACTGCGCAGAACAGAAAGAGCCAPAAAAGAGCTGGAAG |
| | TTCATACAGAAGCTCTAGCTGCTGAAGCATCTAAAGACAGGAAGCTACGTGAACAGAG |
| | TGAGCACTATTCTAAGCAACTGGAAAATGAATTGGAGGGACTGAAGCAAAAACAAATT |
| | AGTTACTCACCAGGAGTATGCAGCATAGAACATCAGCAAGAGATAACCAAACTAAAGA |
| | CTGATTTGGAAAAGAAAAGTATCTTTTATGAAGAAGAATTATCTAAAAGAGAAGGAAT |
| | ACATGCAAATGAAATAAAAAATCTTAAGAAAGAACTGCATGATTCAGAAGCTCAGCAA |
| | CTTGCTCTCAACAAAGAAATTATGATTTTAAAAGACAAATTGGAAAAAACCAGAAGAG |
| | AAAGTCAAAGTGAAAGGGAGGAATTTGAAAGTGAGTTCAAACAACAATATGAACGAGA |
| | AAAAGTGTTGTTAACTGAAGAAAATAAAAAGCTGACGAGTGAACTTGATAAGCTTACT |
| | ACTTTGTATGAGAACTTAAGTATACACAACCAGCAGTTAGAAGAAGAGGTTAAAGATC |
| | TAGCAGACAAGAAAGAATCAGTTGCACATTGGGAAGCCCAAATCACAGAAATAATTCA |
| | GTGGGTCAGCGATGPAAAGGATGCACGATGGTATCTTCAGGCCTTAGCTTCTAAAATG |
| | ACTGAAGAATTGGAGGCATTAAGAAATTCCAGCTTGGGTACACGAGCAACAGTAAGCT |
| | TCTATGATATGCCCTGGAAAATGCGTCGTTTTGCGAAACTGGATATGTCAGCTAGACT |
| | GGAGTTGCAGTCGGCTCTGGATGCAGAAATAAGAGCCAAACAGGCCATCCAAGAAGAG |
| | TTGAATAAAGTTAAAGCATCTAATATCATAACAGAAAAACTAAAAGATTCAGAGAAGA |
| | AGAACTTGGAACTACTCTCAGAAATCGAACAGCTGATAAAGGACACTGAAGAGCTTAG |
| | ATCTGAAAAGGGTATGGAGCACCAAGACTCACAGCATTCTTTCTTGGCATTTTTGAAT |
| | ACGCCTACCGATGCTCTGGATCAATTTGAATCTCCATCCTGTACTCCAGCTAGCAAAG |
| | GCAGACGTGTAAGAGACTCCACTCCACTTTCAGTTCACACACCAACCTTAAGGAAAAA |
| | AGGATGTCCTGGTTCAACTGGCTTTCCACCTAAGCGCAAGACTCACCAGTTTTTTGTA |
| | AAATCTTTTACTACTCCTACCAAGTGTCATCAGTGTACCTCCTTGATGGTGGGTTTAA |
| | TAAGACAGGGCTGTTCATGTGAAGTGTGTGGATTCTCATGCCATATAACTTGTGTAAA |
| | CAAAGCTCCAACCACTTGTCCAGTTCCTCCTGAACAGACAAAAGGTCCCCTCGGTATA |
| | GATCCTCAGAAAGGAATAGGAACAGCATATGAAGGTCATGTCAGGATTCCTAAGCCAG |
| | CTGGAGTGAAGAAGGGTGGCAGAGAGCACTGGCTATAGTGTGTGACTTCAAAACTCTT |
| | TCTGTACGATATTGCTGGAGGAAAAGCATCTCAGCCCAGTGTTGTCATTAGTCAAGTG |
| | ATTGACATGAGGGATGAAGAATTTTCTGTGAGTTCAGTCTTGGCTTCTGATGTTATCC |
| | ATGCAAGTCGAAAAGATATACCCTGTATATTTAGGGTCACAGCTTCCCAGCTCTCAGC |
| | ATCTAATAACAAATGTTCAATCCTGATGCTAGCAGACACTGAGAATGAGAAGAATAAG |
| | TGGGTGGGAGTGCTGAGTGAATTGCACAAGATTTTGAAGAAAAACAAATTCAGAGACC |
| | GCTCAGTCTATGTTCCCAAAGAGGCTTATGACAGCACTCTACCCCTCATTAAAACAAC |
| | CCAGGCAGCCGCAATCATAGATCATGAAGAATTGCTTTGGGAACGAAAGAAAGGGTTA |
| | TTTGTTGTACATGTCACCAAGATGAATTATTAGAGTTGGTGACAATAAAGAAAGATTC |
| | ATCAGATTGAACTCATTCCAAATGATCAGCTTGTTGCTGTGATCTCAGGACGAAATCG |
| | TCATGTACGACTTTTTCCTATGTCAGCATTGGATGGGCGACAGACCGATTTTTACAAG |
| | CTCTCAGAAACTAAAGGGTGTCAAACCGTAACTTCTGGAAAGGTGCGCCATGGAGCTC |
| | TCACATGCCTGTGTGTGGCTATGAAAAGGCAGGTCCTCTGTTATGAACTATTTCAGAG |
| | CAAGACCCGTCACAGAAAATTTAAAGAAATTCAAGTCCCATATAATGTCCAGTGGATG |
| | GCAATCTTCAGTGAACAACTCTGTGTGGGATTCCAGTCAGGATTTCTAAGATACCCCT |
| | TGAATGGAGAGGAAATCCATACAGTATGCTCCATTCAAAATGACCATACACTATCATT |
| | TATTGCACATCAACCAATGGATGCTATCTGCGCAGTTGAGATCTCCAGTAAAGAATAT |
| | CTGCTGTGTTTTAACAGCATTGGGATATACACTGACTGCCAGGGCCGAAGATCTAGAC |
| | AACAGGAATTGATGTGGCCAGCAAATCCTTCCTCTTGTTGTTACAATGCACCATATCT |
| | CTCGGTGTACAGTGAAAATGCAGTTGATATCTTTGATGTGAAACTCCATGGATGGATT |
| | CAGACTCTTCCTCTCAAAAAGGTTCGACCCTTAAACAATGAAGGATCATTAAATCTTT |
| | TAGGGTTGGAGACCATTAGATTAATATATTTCAAATAAAGATGGCAGAAAGGGGACGA |
| | ACTGGTAGTACCTGAAACATCAGATAATAGTCGGAAACAAATGGTTAGAAACATTAAC |
| | AATAAGCGGCGTTATTCCTTCAGAGTCCCAGAAGAGGAAAGGATGCAGCAGAGGAGGG |
| | AAATGCTACGAGATCCAGAATGAGAATAAATTAAATTTCTAAATCCAACTAATTTTAA |
| | TCACATAGCACACATGGGTCCTGGAGATGGAATACAGATCCTGAAAGATCTGCCCATG |
| | AACCCTCGGCCTCAGGAAAGTCGGACAGTATTCAGTGGCTCAGTCAGTATTCCATCTA |
| | TCACCAAATCCCGCCCTGAGCCAGGCCGCTCCATGAGTGCTAGCAGTGGCTTGTCAGC |
| | AAGTAAGTGCCGGGGCTACAGGAAGGTGCCTCTGAGACAGGGTGACCCCCAGCTCCCT |
| | CCCCTCCTGTCCCGTGGGTGACATGTCCTTCACTTACGTGTGCCCATTGCATTCTCAA |
| | GTCGCTGCAGTGTCTCAGACCCTGCTGGGTAATGCCTAATAGGCACAAATGCAGTTGT |
| | TAAGAAAATAGTCCCAGAGTCCTTCTAGAGTGTACAGGCCATCTGGGAGACAGACAAA |
| | TATGATTACAAATTGTGATGATAAGGCTCTGAAGGAAGTAAACAGCATACATTAAATA |
| | GAGAATAACAAGGGGTAGCTGTTAGGGATGAATCCCTACTTGGCAGAATAATTAGGAA |
| | AATCACTCCCTAGAGGTGGAGTCATGTTTGAGTAATGTTTGGTTAACTGAAAGAAGGC |
| | TAGTATGGCTACATGGTAGTGGTGAGGAAGTAACAAAAATTAGAGCGGGGTAGCAGGT |
| | AAGGGTCAGACCAGCAGGGACTTGAAGACCAAGGTAAGACATTTTTTACTTTATTCAA |
| | AAGGAAAAGGAAGACTTTTAAGTAGGGAAGAATTTTCTTTCAATTTACATTCTTAAAA |
| | CAATCCTGCGGGCTGCCAAGTGGAGAATGGACTAGAGGCAGGAAGAGTGGAAGCCAGC |
| | ATCCAGATAGGAGACTCCTAGAGTGGTCCCAATGGAAACCAATGAGGGCTTGGGATGC |
| | AGCAGGGGCAGAAGGAGAGAAGATGGTAGATTCTCCAGATATATTTTCAGAGTTAAAA |
| | GCAGTAAGACTTGATGATGAATTAGTCATGGAAAGTAAGGGAGAGAGTTAAAAGATGA |
| | CTCCCAGACTTCCTGCTAGGGCCTTAGTATGATACCATTTACTCCCATTTACCACCGT |
| | TTAGAAGGGGCTGAGGCAGGACATTCCACGCATGTCCAAAAGGTCCCGAGGTAGCAAA |
| | AAAAAAAAAAAAAAAA |
| | ORE Start: ATG at 15 ORF Stop: TGA at 5007 |
| | SEQ ID NO: 102 1664 aa MW at 190605.2kD |
| NOV32b, | MSGEVRLRQLEQFILDGPAQTNGQCFSVETLLDILICLYDECNNSPLRREKNILEYLE |
| CG128891-02 | |
| Protein | WGAKPFTSKVKQMRLHREDFEILKVIGRGAFGEVAVVKLKNADKVFAMKILNKWEMLK |
| Sequence | |
| | RAETACFREERDVLVNGDNKWITTLHYAFQDDNNLYLVMDYYVGGDLLTLLSKFEDRL |
| | PEDMARFYLAEMVIAIDSVHQLHYVHRDTKPDNILMDMNGHIRLADPGSCLKLMEDGT |
| | VQSSVAVGTPDYISPEILQAMEDGKGRYGRECDWWSLGVCMYEMLYGETPFYAESLVE |
| | TYGKTMNHKERPQPPAQVTDVSENAKDLIRRLICSREHRLGQNGIEDFKKHPFFSGID |
| | WDNIRNCEAPYTPEVSSPTDTSNFDVDDDCLKNSETMPPPTHTAPSGHHLPPVGFTYT |
| | SSCVLSDRSCLRVTAGPTSLDLDVNVQRTLDNNLATEAYERRIKRLEQEKLELSRKLQ |
| | ESTQTVQALQYSTVDGPLTASKDLFIKNLKEEIEKLRKQVTESSHLEQQLEEANAVRQ |
| | ELDDAPRQIKAYEKQIKTLQQEREDLNKELVQASERLKNQSKELKDAHCQRKLAMQEF |
| | MEINERLTELHTQKQKLARHVRDKEEEVDLVMQKVESLRQELRRTERAKKELEVHTEA |
| | LAAEASKDRKLREQSEHYSKQLENELFGLKQKQISYSPGVCSIEHQQEITKLKTDLEK |
| | KSIFYEEELSKREGIHANETKNLKKELHDSEGQQLALNKEIMILKDKLEKTRRESQSE |
| | REEFESEFKQQYEREKVLLTFENKKLTSELDKLTTLYENLSTHNQQLEEEVKDLADKK |
| | ESVAHWEAQITEIIQWVSDEKDARWYLQALASKMTEELEALRNSSLGTRATVSFYDMP |
| | WKMRRFAKLDMSARLELQSALDAEIRAKQAIQEELNKVKASNIITEKLKDSEKKNLEL |
| | LSEIEQLIKDTEELRSEKGMEHQDSQHSFLAPLNTPTDALDQFESPSCTPASKGRRVR |
| | DSTPLSVHTPTLRKKGCPGSTGFRRKRKTHQFFVKSPTTPTKCHQCTSLMVGLIRQGC |
| | SCEVCGFSCHITCVNKAPTTCRVPPEQTKGPLGIDPQKGIGTAYEGHVRIPKPAGVKK |
| | GWQRALAIVCDFKLFLYDIAGGKASQPSVVISQVIDMRDEEFSVSSVLASDVTHASRK |
| | DIPCIFRVTASQLSASNNKCSILMLADTENEKNKWVGVLSELHKILKKNKFRDRSVYV |
| | PKEAYDSTLRLIKTTQAAAIIDHERIALGNEEGLPVVHVTKDEIIRVGDNKKIHQIEL |
| | IPNDQLVAVISGRNRHVRLFRMSALDGRETDFYKLSETKGCQTVTSGKVRHGALTCLC |
| | VAMKRQVLCYELFQSKTRHRKPKEIQVPYNVQWMAIFSEQLCVGFQSCPLRYPLNGEG |
| | NPYSMLHSNDHTLSFIAHQPMDAICAVEISSKEYLLCFNSIGIYTDCQGRRSRQQELM |
| | WPANPSSCCYNARYLSVYSENAVDIFDVNSMEWIQTLPLKKVRPLNNEGSLNLLGLET |
| | IRLIYFKNKMAEGDELVVRFTSDNSRKQMVRNINNKRRYSFRVPEEERMQQRREMLRD |
| | PEMRNKLISNPTNFNHIAHMGPGDGIQILKDLPMNRRPQESRTVFSGSVSIPSITKSR |
| | PEPGRSMSASSGLSASKCRGYRKVPLRQGDPQLPPLLSRG |
| | SEQ ID NO: 103 874 bp |
| NOV32c, | CACCGGATCCAAAACAACCCAGGCAGCCGCAATCATAGATCATGAAAGAATTGCTTTG |
| 276585662 | |
| DNA | GGAACGAAGAAGGGTTATTTGTTGTACATGTCACCAAAGATGAAATTAATTAGAGTTG |
| Sequence | |
| | GTGACAATAAGAAGATTCATCAGATTGAACTCATTCCAAATGATCAGCTTGTTGCTGT |
| | GATCTCAGGACGAAATCGTCATGTACGACTTTTTCCTATCTCAGCATTGGATGGGCGA |
| | GAGACCGATTTTTACAAGCTGTCAGAAACTAAAGGGTGTCAAACCGTAACTTCTGGAA |
| | AGGTGCGCCATGGAGCTCTCACATGCCTGTGTGTGGCTATGAAAAGGCAGGTCCTCTG |
| | TTATGAACTATTTCAGAGCAAGACCCGTCACAGAAAATTTAAAGAAATTCAAGTCACA |
| | TATAATGTCCAGTGGATGGCAATCTTCAGTGAACAACTCTGTGTGGGATTCCAGTCAG |
| | GATTTCTAAGATACCCCTTGAATGGAGAAGGAAATCCATACAGTATGCTCCATTCAAA |
| | TGACCATACACTATCATTTATTGCACATCAACCAATGGATGCTATCTGCGCAGTTGAG |
| | ATCTCCAGTAAAGAATATCTGCTGTGTTTTAACAGCATTGGGATATACACTGACTGCC |
| | AGGGCCGAAGATCTAGACAACAGGAATTGATGTGGCCAGCAAATCCTTCCTCTTGTTG |
| | TTACAATGCACCATATCTCTCGGTGTACAGTGAAAATGCAGTTGATATCTTTGATGTG |
| | AACTCCATGGAATGGATTCAGACTCTTCCTCTCAAAAAGGTTCGACCCTTAAACAATG |
| | AAGGATCATTAATCTTTTAGGGTTGGAGACCATTAGATTAAATATATTTCAAACTCGA |
| | GGGC |
| | ORE Start: at 2 ORE Stop: end of sequence |
| | SEQ ID NO: 104 291 aa MW at 33043.6kD |
| NOV32c, | TGSKTTQAAAIIDHERIALGNEEGLFVVHVTKDEIIRVGDNKKIHQIELTPNDQLVAV |
| 276585662 | |
| Protein | LSGRNRHVRLFPMSALDGRETDFYKLSETKGCQTVTSGKVRHGALTCLCVAMKRQVLC |
| Sequence | |
| | YELFQSKTRHRKFKEIQVPYNVQWMATFSEQLCVGFQ8GELRYPLNGEGNPYSMLHSN |
| | DHTLSFIAHQPMDAICAVEISSKEYLLCFNSIGIYTDCQGRRSRQQELMWPANPSSCC |
| | YNAPYLSVYSENAVDIFDVNSMEWIQTLPLKKVRPLNNEGSLNLLGLETIRLIYFKLE |
| | G |

Sequence comparison of the above protein sequences yields the following sequence relationships shown in Table 32B.

TABLE 32B
Comparison of NOV32a against NOV32b and NOV32c

| | | Identities/ |
|---|---|---|
| Protein | NOV32a Residues/ | Similarities for |
| Sequence | Match Residues | the Matched Region |
| NOV32b | 1 . . . 1633 | 1566/1633 (95%) |
| | 1 . . . 1629 | 1566/1633 (95%) |
| NOV32c | 1232 . . . 1519 | 272/288 (94%) |
| | 4 . . . 288 | 272/288 (94%) |

Further analysis of the NOV32a protein yielded the following properties shown in Table 32C.

TABLE 32C
Protein Sequence Properties NOV32a

| | |
|---|---|
| PSort | 0.9800 probability located in nucleus; 0.3000 probability |
| analysis: | located in microbody (peroxisome); 0.1000 probability located |
| | in mitochondrial matrix space; 0.1000 probability located in |
| | lysosome (lumen) |
| SignalP | No Known Signal Sequence Predicted |
| analysis: | |

A search of the NOV32a protein against the Geneseq database, a proprietary database that contains sequences published in patents and patent publication, yielded several homologous proteins shown in Table 32D.

TABLE 32D
Geneseq Results for NOV32a

| | | NOV32a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAE21707 | Human PKIN-2 protein-Homo | 1 . . . 1738 | 1712/1740 (98%) | 0.0 |
| | sapiens, 1719 aa. [WO200218557-A2, | 1 . . . 1719 | 1714/1740 (98%) | |
| | 7 Mar. 2002] | | | |
| AAB42069 | Human ORFX ORF1833 polypeptide | 1 . . . 1737 | 1062/1779 (59%) | 0.0 |
| | sequence SEQ ID NO: 3666-Homo | 1 . . . 1727 | 1349/1779 (75%) | |
| | sapiens, 1728 aa. [WO200058473-A2, | | | |
| | 5 Oct. 2000] | | | |
| ABG13880 | Novel human diagnostic protein | 1 . . . 1602 | 1017/1626 (62%) | 0.0 |
| | #13871-Homo sapiens, 1797 aa. | 1 . . . 1607 | 1286/1626 (78%) | |
| | [WO200175067-A2, 11 Oct. 2001] | | | |
| ABB13880 | Novel human diagnostic protein | 1 . . . 1602 | 1017/1626 (62%) | 0.0 |
| | #13871-Homo sapiens, 1797 aa. | 1 . . . 1607 | 1286/1626 (78%) | |
| | [WO200175067-A2, 11 Oct. 2001] | | | |
| ABB60342 | Drosophila melanogaster polypeptide | 21 . . . 1627 | 684/1688 (40%) | 0.0 |
| | SEQ ID NO 7818-Drosophila | 44 . . . 1599 | 988/1688 (58%) | |
| | melanogaster, 1637 aa. | | | |
| | [WO200171042-A2, 27 Sep. 2001] | | | |

In a BLAST search of public sequence databases, the NOV32a protein was found to have homology to the proteins shown in the BLASTP data in Table 32E.

TABLE 32E
Public BLASTP Results for NOV32a

| | | NOV32a | Identities/ | |
|---|---|---|---|---|
| Protein | | Residues/ | Similarities for | |
| Accession | | Match | the Matched | Expect |
| Number | Protein/Organism/Length | Residues | Portion | Value |
| O54874 | Mytonic dystrophy kinase-related | 1 . . . 1738 | 1657/1741 (95%) | 0.0 |
| | Cdc42-binding kinase-Rattus | 1 . . . 1732 | 1700/1741 (97%) | |
| | norvegicus (Rat), 1732 aa. | | | |
| Q9ULU5 | K1AA1124 protein-Homo sapiens | 1 . . . 1737 | 1062/1762 (60%) | 0.0 |
| | (Human), 1760 aa (fragment). | 50 . . . 1759 | 1349/1762 (76%) | |
| Q9Y5S2 | CDC42-binding protein kinase beta- | 1 . . . 1737 | 1061/1762 (60%) | 0.0 |
| | Homo sapiens (Human), 1711 aa. | 1 . . . 1710 | 1349/1762 (76%) | |
| O54875 | Myotonic dystrophy kinase-related | 1 . . . 1726 | 1050/1748 (60%) | 0.0 |
| | Cdc42-binding kinase MRCK-beta- | 1 . . . 1702 | 1334/1748 (76%) | |
| | Rattus norvegicus (Rat), 1702 aa. | | | |
| Q9W1B0 | GEK protein (LD24220P)- | 21 . . . 1627 | 684/1688 (40%) | 0.0 |
| | Drosophila melanogaster (Fruit fly), | 44 . . . 1599 | 988/1688 (58%) | |
| | 1637 aa. | | | |

PFam analysis predicts that the NOV32a protein contains the domains shown in the Table 32F.

TABLE 32F
Domain Analysis of NOV32a

| | | Identities/ | |
|---|---|---|---|
| Pfam | NOV32a | Similarities for | Expect |
| Domain | Match Region | the Matched Region | Value |
| Pkinase | 78 . . . 344 | 86/303 (28%) | 1.9e−61 |
| | | 205/303 (68%) | |
| pkinase_C | 345 . . . 373 | 16/31 (52%) | 2.3e−08 |
| | | 25/31 (81%) | |
| K-box | 468 . . . 554 | 23/105 (22%) | 0.34 |
| | | 54/105 (51%) | |
| DAG_PE-bind | 1016 . . . 1065 | 21/51 (41%) | 1.9e−14 |
| | | 38/51 (75%) | |
| PH | 1086 . . . 1205 | 16/120 (13%) | 1.2e−06 |
| | | 82/120 (68%) | |
| CNH | 1232 . . . 1519 | 64/381 (17%) | 1.4e−11 |
| | | 188/381 (49%) | |

### Example 33

The NOV33 clone was analyzed and the nucleotide and encoded polypeptide sequences are shown in Table 33A.

TABLE 33A
NOV33 Sequence Analysis

| | |
|---|---|
| | SEQ ID NO: 105 3117 bp |
| NOV33a. | TAGGAGTGAACACTGCACAGGAATCTCTGCCCATCTCAGGAGAAACCAAACTTGGGGA |
| CG131490-01 | |
| DNA | AAATGTTTGCGGTCCACTTGATGGCATTTTACTTCAGCAAGCTGAAGGAGGACCAGAT |
| Sequence | |
| | CAAGAAGGTGGACAGGTTCCTGTATCACATGCGGCTCTCCGATGACACCCTTTTGGAC |
| | ATCATGAGGCGGTTCCGGGCTGAGATGGAGAAGGGCCTGGCAAAGGACACCAACCCCA |
| | CGGCTGCAGTGAAGATGTTGCCCACCTTCGTCAGGGCCATTCCCGATGGTTCCGAAAA |
| | TGGGGAGTTCCTTTCCCTGGATCTCGGAGGGTCCAAGTTCCGAGTGCTGAAGGTGCAA |
| | GTCGCTGAAGAGGGGAAGCGACACGTGCAGATGGAGAGTCAGTTCTACCCAACGCCCA |
| | ATGAAATCATCCGCGGGAACGGCACAGAGCTGTTTGAATATGTAGCTGACTGTCTGGC |
| | AGATTTCATGAAGACCNAGATTTAAGCATAAGAAAAATTGCCCCTTGGCCTAACTTTT |
| | TCTTTCCCCTGTCGACAGACTAAACTGGAAGAGGGTGTCCTACTTTCGTGGACAAAAA |
| | AGTTTAAGGCACGAGGAGTTCAGGACACGGATGTGGTGAGCCGTCTGACCAAAGCCAT |
| | GAGAAGACACAAGGACATGGACGTGGACATCCTGGCCCTGGTCAATGACACCGTGGGG |
| | ACCATGATGACCTGTGCCTATGACGACCCCTACTGCGAAGTTGGTGTCATCATCGGAA |
| | CTGGCACCAATGCGTGTTACATGGAGGACATGAGCAACATTGACCTGGTGGAGGGCGA |
| | CGAGGGCAGGATGTGCATCAACACAGAGTGGGGGGCCTTCGGGGACGACGGGGCCCTG |
| | GAGGACATTCGCACTGAGTTCGACAGGGAGCTGGACCTCGGCTCTCTCAACCCAGGAA |
| | AGCAACTGTTCGAGAAGATGATCAGTGGCCTGTACCTGGGGGAGCTTGTCAGGCTTAT |
| | CTTGCTGAAGATGGCCAAGGCTGGCCTCCTGTTTGGTGGTGAGAAATCTTCTGCTCTC |
| | CACACTAAGGGCAAGATCGAAACACGGCACGTGGCTGCCATGGAGAAGTATAAAGAAG |
| | GCCTTGCTAATACAAGAGAGATCCTGGTGGACCTGGGTCTGGAACCGTCTGAGGCTGA |
| | CTGCATTGCCGTCCAGCATGTCTGTACCATCGTCTCCTTCCGCTCGGCCAATCTCTGT |
| | GCAGCAGCTCTGGCGGCCATCCTGACACGCCTCCGGGAGAACAAGAAGGTGGAACGGC |
| | TCCGGACCACAGTGGGCATGGACGGCACCCTCTACAAGATACACCCTCAGTACCCAAA |
| | ACGCCTGCACAAGGTGGTGAGGAAACTGGTCCCAAGCTGTGATGTCCGCTTCCTCCTG |
| | TCAGAGAGTGGCAGCACCAAGGGGGCCGCCATGGTGACCGCGGTGGCCTCCCGCGTGC |
| | AGGCCCAGCGGAAGCAGATCGACAGGGTGCTGGCTTTGTTCCAGCTGACCCGAGAGCA |
| | GCTCGTGGACGTGCAGGCCAAGATGCGGGCTGAGCTGGAGTATGGGCTGAAGAAGAAG |
| | AGCCACGGGCTGGCCACGGTCAGGATGCTGCCCACCTACGTCTGCGGGCTGCCGGACG |
| | GCACAGAGAAAGGAAAGTTTCTCGCCCTGGATCTTGGGGGAACCAACTTCCGGGTCCT |
| | CCTGGTGAAGATCAGAAGTGGACGGAGGTCAGTGCGPATGTACAACAAGATCTTCGCC |
| | ATCCCCCTGGAGATCATGCAGGGCACTGGTGAGGAGCTCTTTGATCACATTGTGCAGT |
| | GCATCGCCGACTTCCTGGACTACATGGGCCTCAAGGGAGCCTCCCTACCTTTGGGCTT |
| | CACATTCTCATTTCCCTGCAGGCAGATGAGCATTGACAAGGGAACACTCATAGGGTGG |
| | ACCAAAGGTTTCAAGGCCACTGACTGTGAAGGGGAGGACGTGGTGGACATGCTCAGGG |
| | AAGCCATCAAGAGGAGAAACGAGTTTGACCTGGACATTGTTGCAGTCGTGAATGATAC |
| | AGTGGGGACCATGATGACCTGTGGCTATGAAGATCCTAATTGTGAGATTGGCCTGATT |
| | GCAGGAACAGGCAGCAACATGTGCTACATGGAGGACATGAGGAACATCGAGATGGTGG |
| | AGGGGGGTGAAGGGAAGATGTGCATCAATACAGAGTGGGGAGGATTTGGAGACAATGG |
| | CTGCATAGATGACATCTGGACCCGATACGACACGGAGGTGGATGAGGGGTCCTTGAAT |
| | CCTGGCAAGCAGAGATACGAGAAAATGACCAGTGGGATGTACTTGGGGGAGATTGTGC |
| | GGCAGATCCTGATCGACCTGACCAAGCAGGGTCTCCTCTTCCGAGGGCAGATTTCAGA |
| | GCGTCTCCGGACCAGGGGCATCTTCGAAACCAAGTTCCTGTcCCAGATCGAAAGCGAT |
| | CGGCTGGCCCTTCTCCAGGTCAGGAGGATTCTGCAGCAGCTGGGCCTGGACAGCACGT |
| | GTGAGGACAGCATCGTGGTGAAGGAGGTGTGCGGACGCGTGTCCCGGCGGGCGGCCCA |
| | GCTCTGCGGTGCTGGCCTGGCCGCTATAGTGGAAAAAAGGAGAGAAGACCAGGGGCTA |
| | GAGCACCTGAGGATCACTGTGGGTGTGGACGGCACCCTGTACAAGCTGCACCCTCACT |
| | TTTCTAGAATATTGCAGGAAACTGTGAAGGAACTAGCCCCTCGATGTGATGTGACATT |
| | CATGCTGTCAGAAGATGGCAGTGGAAAAGGGGCAGCACTGATCACTGCTGTGGCCAAG |
| | AGGTTACAGCAGGCACAGAAGGAGAACTAGGAACCCCTGGGATTGGACCTGATGCATC |
| | TTGGATACTGAACAGCTTTTCCTCTGGCAGATCAGTTGGTCAGAGAGCAATCGGCACC |
| | CTCCTGGCTGACCTCACCTTCTGGATGGCCGAAAGAGAACCCCAGGTTCTCGGGTACT |
| | CTTAGTATCTTGTACTGGATTTGCAGTGACATTACATGACATCTCTATTTGGTATATT |
| | TGGGCCAAAATGGGCCAACTTATGAAATCAAAGTGTCTGTCCTGAGAGATCCCCTTTC |
| | AACACATTGTTCAGGTGAGGCTTGAGCTGTCAATTCTCTATGG |
| | ORF Start: ATG at 61 ORF Stop: TAG at 2812 |
| | SEQ ID NO: 106 917 aa MW at 102628.8kD |
| NOV33a. | MFAVHLMAFYFSKLKEDQIKKVDRFLYHMRLSDDTLLDIMRRFRAEMEKGLAKDTNPT |
| CG131490-01 | |
| Protein | AAVKMLPTFVRAIPDGSENGEFLSLDLGGSKFRVLKVQVAEEGKRHVQMESQFYPTPN |
| Sequence | |
| | EIIRGNGTELFEYVADCLADFMKTKDLKHKKLPLGLTFSFPCRQTKLEEGVLLSWTKK |
| | FKARGVQDTDVVSRLTKAMRRHKDMDVDILALVNDTVGTMMTCAYDDPYCEVGVIIGT |
| | GTNACYMEDMSNIDLVEGDEGRMCINTEWGAPGDDGALEDIRTEFDRELDLGSLNPGK |
| | QLFEKMTSGLYLGELVRLILLKMAKAGLLPGGEKSSALHTKGKIETRHVAAMEKYKEG |
| | LANTREILVDLGLEPSEADCIAVQHVCTIVSFRSANLCAAALAATLTRLRENKKVERL |
| | RTTVGMDGTLYKIHRQYPKRLHKVVRKLVPSCDVRFLLSESGSTKGAAMVTAVASRVQ |
| | AQRKQTDRVLALFQLTREQLVDVQAKMRAELFYGLKKKSHGLATVRMLPTYVCGLPDG |
| | TEKGKFLALDLGGTNFRVLLVKIRSGRRSVRMYNKIFAIPLEIMQGTGEELFDHIVQC |
| | TADPLDYMGLKGASLPLGFTFSFPCRQMSIDKGTLIGWTKGFKATDCEGEDVVDMLRE |
| | ATKRRNEFDLDIVAVVNDTVGTMMTCGYEDPNCEIGLIAGTGSNMCYMEDMRNIEMVE |
| | GGEGKMCTNTEWGGFGDNGCTDDTWTRYDTEVDEGSLNPGKQRYEKMTSGMYLGETVR |
| | QILIDLTKQGLLFRGQISERLRTRGIFETKFLSQIESDRLALLQVRRILQQLGLDSTC |
| | EDSIVVKEVCGRVSRRAAQLCGAGLAATVEKRREDQGLEHLRITVGVDGTLYKLHPHF |
| | SRILQETVKELAPRCDVTFMLSEDGSGKGAALTTAVAKRLQQAQKEN |
| | SEQ ID NO: 107 2277 bp |
| NOV33b, | TAGGAGTGAACACTGCACAGGAATCTCTGCCCATCTCAGGAGAAACCAAACTTGGGGA |
| CG131490-02 | |
| DNA | AAATGTTTGCGGTCCACTTGATGGCATTTTACTTCAGCAAGCTGAAGGAGGACCAGAT |
| Sequence | |
| | CAAGAAGGTGGACAGGTTCCTGTATCACATGCGGCTCTCCGATGACACCCTTTTGGAC |
| | ATCATGAGGCGGTTCCGGGCTGAGATGGAGAAGGGCCTGGCAAAGGACACCAACCCCA |
| | CGGCTGCAGTGAAGATGTTGCCCACCTTCGTCAGGGCCATTCCCGATGGTTCCGAAAA |
| | TGGGGAGTTCCTTTCCCTGGATCTCGGAGGGTCCAAGTTCCGAGTGCTGAAGGTGCAA |
| | GTCGCTGAAGAGGGGAAGCGACACGTGCAGATGGAGAGTCAGTTCTACCCAACGCCCA |
| | ATGAAATCATCCGCGGGAACGGCACAGAGCTGTTTGAATATGTAGCTGACTGTCTGGC |
| | AGATTTCATGAAGACCAAAGATTTAAAGCATAAGAAATTGCCCCTTGGCCTAACTTTT |
| | TCTTTCCCCTGTCGACAGACTAAACTGGAAGAGGGTGTCCTACTTTCGTGGACAAAAA |
| | AGTTTAAGGCACGAGGAGTTCAGGACACGGATGTGGTGAGCCGTCTGACCAAAGCCAT |
| | GAGAAGACACAAGGACATGGACGTGGACATCCTGGCCCTGGTCAATGACACCGTGGGG |
| | ACCATGATGACCTGTGCCTATGACGACCCCTACTGCGAAGTTGGTGTCATCATCGGAA |
| | CTGGCACCAATGCGTGTTACATGGAGGACATGAGCAACATTGACCTGGTGGAGGGCGA |
| | CGAGGGCAGGATGTGCATCAACACAGAGTGGGGGGCCTTCGGGGACGACGGGGCCCTG |
| | GAGGACATTCGCACTGAGTTCGACAGGGAGCTGGACCTCGGCTCTCTCAACCCAGGAA |
| | AGCAACTGTTCGAGAAGATGATCAGTGGCCTGTACCTGGGGGAGCTTGTCAGGCTTAT |
| | CTTGCTGAAGATGGCCAAGGCTGGCCTCCTGTTTGGTGGTGAGAAATCTTCTGCTCTC |
| | CACACTAAGGGCAAGATCGAACACGGCACGTGGCTGCCATGGAGAAGTATAAAAGAAG |
| | GCCTTGCTAATACAAGAGAGATCCTGGTGGACCTGGGTCTGGAACCGTCTGAGGCTGA |
| | CTGCATTGCCGTCCAGCATGTCTGTACCATCGTCTCCTTCCGCTCGGCCAATCTCTGT |
| | GCAGCAGCTCTGGCGGCCATCCTGACACGCCTCCGGGAGAACAAGAAGGTGGAACGGC |
| | TCCGGACCACAGTGGGCATGGACGGCACCCTCTACAAGATACACCCTCAGTACCCAAA |
| | ACGCCTGCACAAGGTGGTGAGGAAACTGGTCCCAAGCTGTGATGTCCGCTTCCTCCTG |
| | TCAGAGAGTGGCAGCACCAAGGGGGCCGCCATGGTGACCGCGGTGGCCTCCCGCGTGC |
| | AGGCCCAGCGGAAGCAGATCGACAGGGTGCTGGCTTTGTTCCAGCTGACCCGAGAGCA |
| | GCTCGTGGACGTGCAGGCCAAGATGCGGGCTGAGCTGGAGTATGGGCTGAAGAAGAAG |
| | AGCCACGGGCTGGCCACGGTCAGGATGCTGCCCACCTACGTCTGCGGGCTGCCGGACG |
| | GCACAGAGAAAGGAAAGTTTCTCGCCCTGGATCTTGGGGGAACCAACTTCCGGGTCCT |
| | CCTGGTGAAGATCAGAAGTGGACGGAGGTCAGTGCGAATGTACAACAAGATCTTCGCC |
| | ATCCCCCTGGAGATCATGCAGGGCACTGGTGAGGAGCTCTTTGATCACATTGTGCAGT |
| | GCATCGCCGACTTCCTGGACTACATGGGCCTCAAGGGAGCCTCCCTACCTTTGGGCTT |
| | CACATTCTCATTTCCCTGCAGGCAGATGAGCATTGACAAGGGAACACTCATAGGGTGG |
| | ACCAAAGGTTTCAAGGCCACTGACTGTGAAGGGGAGGACGTGGTGGACATGCTCAGGG |
| | AAGCCATCAAGAGGAGAAACGAGTTTGACCTGGACATTGTTGCAGTCGTGAATGATAC |
| | AGTGGGGACCATGATGACCTGTGGCTATGAAGATCCTAATTGTGAGATTGGCCTGATT |
| | GCAGGAACAGGCAGCAACATGTGCTACATGGAGGACATGAGGAACATCGAGATGGTGG |
| | AGGGGGGTGAAGGGAAGATGTGCATCTGTTTTTCATTTTGCCTGTGGTTTGTGTTGCA |
| | GGTGTTGATAGTTGTTTTAAGGATTGTTAGGTATAGGAAATCCAGTAAATTAATAAAA |
| | AAATTTTGATTTTCC |
| | ORF Start: ATG at 61 ORF Stop: TGA at 2269 |
| | SEQ ID NO: 108 736 aa MW at 82680.6kD |
| NOV33b, | MFAVHLMAFYFSKLKEDQTKKVDRFLYHMRLSDDTLLDTMRRFRAEMEKGLAKDTNPT |
| CG131490-02 | |
| Protein | AAVKMLPTFVRAIPDGSENGEFLSLDLGGSKFRVLKVQVAEEGKRHVQMESQFYPTPN |
| Sequence | |
| | EIIRGNGTELFEYVADCLADFMKTKDLKHKKLPLGLTFSFPCRQTKLEEGVLLSWTKK |
| | FKARGVQDTDVVSRLTKAMRRHKDMDVDILALVNDTVGTMMTCAYDDPYCEVGVIIGT |
| | GTNACYMEDMSNTDLVEGDEGRMCINTEWGAFGDDGALEDIRTEFDRELDLGSLNPGK |
| | QLPEKMISGLYLGELVRLILLKMAKAGLLFGGEKSSALHTKGKTETRHVAAMEKYKEG |
| | LANTREILVDLGLEPSEADCIAVQHVCTIVSPRSANLCAAALAAILTRLRENKKVERL |
| | RTTVGMDGTLYKIHPQYPKRLHKVVRKLVPSCDVRFLLSESGSTKGAAMVTAVASRVQ |
| | AQRKQIDRVLALFQLTREQLVDVQAKMRAELEYGLKKKSHGLATVRMLPTYVCGLPDG |
| | TEKGKFLALDLGGTNFRVLLVKIRSGRRSVRMYNKIFAIPLEIMQGTGEELFDHIVQC |
| | IADFLDYMGLKGASLPLGFTFSPPCRQMSIDKGTLIGWTKGFKATDCEGEDVVDMLRE |
| | AIKRRNEFDLDIVAVVNDTVGTMMTCGYEDPNCEIGLIAGTGSNMCYMEDMRNIEMVE |
| | GGEGKMCTCFSFCLWFVLQVLIVVLRIVRYRKSSKLIKKF |

Sequence comparison of the above protein sequences yields the following sequence relationships shown in Table 33B.

TABLE 33B
Comparison of NOV33a against NOV33b

| | | Identities/ |
|---|---|---|
| Protein | NOV33a Residues/ | Similarities for |
| Sequence | Match Residues | the Matched Region |
| NOV33b | 1 . . . 704 | 689/704 (97%) |
| | 1 . . . 704 | 689/704 (97%) |

Further analysis of the NOV33a protein yielded the following, properties shown in Table 33C.

TABLE 33C
Protein Sequence Properties NOV33a

| | |
|---|---|
| PSort | 0.6000 probability located in nucleus; 0.3000 probability |
| analysis: | located in microbody (peroxisome); 0.1000 probability located |
| | in mitochondrial matrix space; 0.1000 probability located in |
| | lysosome (lumen) |
| SignalP | Cleavage site between residues 18 and 19 |
| analysis: | |

A search of the NOV33a protein against the Geneseq database, a proprietary database that contains sequences published in patents and patent publication, yielded several homologous proteins shown in Table 33D.

TABLE 33D
Geneseq Results for NOV33a

| | | NOV33a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAE19159 | Human kinase polypeptide (PKIN-17)- | 1 . . . 917 | 915/917 (99%) | 0.0 |
| | Homo sapiens, 917 aa. | 1 . . . 917 | 915/917 (99%) | |
| | [WO200208399-A2, 31 Jan. 2002] | | | |
| ABB04582 | Human hexokinase 50365-Homo | 1 . . . 917 | 914/917 (99%) | 0.0 |
| | sapiens, 917 aa. [WO200190325-A2, | 1 . . . 917 | 914/917 (99%) | |
| | 29 Nov. 2001] | | | |
| ABB97216 | Novel human protein SEQ ID NO: 484- | 1 . . . 911 | 649/912 (71%) | 0.0 |
| | Homo sapiens, 917 aa. | 1 . . . 912 | 781/912 (85%) | |
| | [WO200222660-A2, 21 Mar. 2002] | | | |
| AAW37428 | Rat hexokinase I- Rattus sp, 918 aa. | 1 . . . 911 | 638/912 (69%) | 0.0 |
| | [WO9726357-A1, 24 Jul. 1997] | 1 . . . 912 | 780/912 (84%) | |
| AAW37442 | Rat hexokinase I- Rattus sp, 918 aa. | 1 . . . 911 | 638/912 (69%) | 0.0 |
| | [WO9726322-A2, 24 Jul. 1997] | 1 . . . 912 | 780/912 (84%) | |

In a BLAST search of public sequence datbases, the NOV33a protein was found to have homology to the proteins shown in the BLASTP data in Table 33E.

TABLE 33E
Public BLASTP Results for NOV33a

| | | NOV33a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD19394 | Sequence 1 from Patent WO0190325- | 1 . . . 917 | 914/917 (99%) | 0.0 |
| | Homo sapiens (Human), 917 aa. | 1 . . . 917 | 914/917 (99%) | |
| Q91W97 | Similar to hexokinase 1-Mus | 1 . . . 916 | 834/916 (91%) | 0.0 |
| | musculus (Mouse), 915 aa. | 1 . . . 914 | 882/916 (96%) | |
| P19367 | Hexokinase,type I (EC 2.7.1.1) (HK | 1 . . . 911 | 648/912 (71%) | 0.0 |
| | 1) (Brain form hexokinase)-Homo | 1 . . . 912 | 781/912 (85%) | |
| | sapiens (Human), 917 aa. | | | |
| P05708 | Hexokinase, type I (EC 2.7.1.1) (HK | 1 . . . 911 | 642/912 (70%) | 0.0 |
| | 1) (Brain form hexokinase)-Rattus | 1 . . . 912 | 782/912 (85%) | |
| | norvegicus (Rat), 918 aa. | | | |
| Q96EH2 | Unknown (Protein for | 241 . . . 917 | 675/677 (99%) | 0.0 |
| | IMAGE: 4563921)-Homo sapiens | 1 . . . 677 | 675/677 (99%) | |
| | (Human), 677 aa (fragment). | | | |

PFam analysis predicts that the NOV33a protein contains the domains shown in the Table 33F.

TABLE 33F
Domain Analysis of NOV33a

| | | Identities/ | |
|---|---|---|---|
| Pfam | NOV33a | Similarities for | Expect |
| Domain | Match Region | the Matched Region | Value |
| hexokinase | 16 . . . 463 | 238/483 (49%) | 7.4e−249 |
| | | 400/483 (83%) | |
| hexokinase | 464 . . . 910 | 264/482 (55%) | 1.8e−280 |
| | | 406/482 (84%) | |

### Example 34

The NOV34 clone was analyzed, and the nucleotide and encoded polypeptide sequences are shown in Table 34A.

TABLE 34A
NOV34 Sequence Analysis

| | |
|---|---|
| | SEQ ID NO: 109 767 bp |
| NOV34a, | TGAACTGAAAATCAGAATCCTGGGCCTCACTCCCAGAGGATCTGATCTACATGTGTGG |
| CG131881-01 | |
| DNA | AGATGCCCAGGAATCTGCTTTATTCTCTTTTGTCCTCCCACCTGTCCCCCCATTTCAG |
| Sequence | |
| | CACCTCGGTAACCTCTGCCAAAGTGGCTGTGAATGGCGTTCAGCTGCATTACCAGCAG |
| | ACTGCAGAGGGAGATCACGCAGTCCTGCTACTTCCTGGGATGTTAGGAAGTGGAGAGA |
| | CTGATTTTGGACCTCAGCTCAGAACCTCAATAAGAAGCTCTTCAACGGTGGTCGCCTG |
| | GGATCCTCGAGGCTATGGACATTCCAGGCCCCCAGATCGCGATTTCCCAGCAGACTTT |
| | TTTGAAAGGGATGCAAAGATGCCTGTTGATTTGATGAAGGCGCTGAAGTTTAAGAAGG |
| | TTTCTCTGCTGGGGTGGAGTGATGGGGGCATAACCGCACTCATTGCTGCTGCAAAATA |
| | TCCATCTTACATCCACAAGATGGTGATCTGGGGCGCCAACGCCTACGTCACTGACGAA |
| | GACAGCATGATATATGAGGGTAAAATCTGCCGGCACCTGCTGCCCCGGGTCCAGTGCC |
| | CCGCCTTGATTGTGCACGGTGAGAAGGATCCTCTGGTCCCACGGTTTCATGCCGACTT |
| | CATTCATAAGCACGTGAAAGGCTCACGGCTGCATTTGATGCCAGAAGGCAAACACAAC |
| | CTGCATTTGCGTTTTGCAGATGAATTCAACAAGTTAGCAGAAGACTTCCTACAATGAG |
| | AATGCACACTCTC |
| | ORF Start: ATG at 61 ORF Stop: TGA at 751 |
| | SEQ ID NO: 110 230 aa MW at 25792.4kD |
| NOV34a, | MPRNLLYSLLSSHLSPHFSTSVTSAKVAVNGVQLHYQQTGEGDHAVLLLRGMLGSGET |
| CG131881-01 | |
| Protein | DFGPQLKNLNKKLFTVVAWDPRGYGHSRPPDRDFPADFFERDAKDAVDLMKALKFKKV |
| Sequence | |
| | SLLGWSDGGITALTAAAKYPSYTHKMVTWGANAYVTDEDSMTYEGNICRHLLPRVQCP |
| | ALIVHGEKDPLVPRFHADFTHKHVKGSRLHLMPEGKHNLHLRFADEFNKLAEDFLQ |
| | SEQ ID NO: 111 953 bp |
| NOV34b. | ATGGAACTGAAAATTCAGAATCCTGGGCCTCACTCCCAGAGGATCTGATCTACATGTG |
| CG131881-03 | |
| DNA | TGGAGATGCCCAGGAATCTGCTTTATTCTCTTTTGTCCTCCCACCTGTCCCCCCATTT |
| Sequence | |
| | CAGCACCTCGGTAACCTCTGCCAAAGTGGCTGTGAATGGCGTTCAGCTGCATTACCAG |
| | CAGACTGGAGAGGGAGATCACGCAGTCCTGCTACTTCCTGGGATGTTAGGAAGTGGAG |
| | AGACTGATTTTGGACCTCAGCTCAAGAACCTCAATAAGAAGCTCTTCACGGTGGTCGC |
| | CTGGGATCCTCGAGGCTATGGACATTCCAGGCCCCCAGATCGCGATTTCCCAGCAGAC |
| | TTTTTTGAAAGGGATGCAAAAGATGCTGTTGATTTGATGAAGGCGCTGAAGTTTAAGA |
| | AGGTTTCTCTGCTGGGGTGGAGTGATGGGGGCATAACCGCACTCATTGCTGCTGCAAA |
| | ATATCCATCTTACATCCACAAGATGGTGATCTGGGGCGCCAACGCCTACGTCACTGAC |
| | GAAGACAGCATGATATATGAGGGCATCCGAGATGTTTCCAAATGGAGTGAGAGAACAA |
| | GAAAGCCTCTAGAAGCCCTCTATGGGTAACATCTGCCGGCACCTGCTGCCCCGGGTCC |
| | AGTGCCCCGCCTTGATTGTGCACGGTGAGAAGGATCCTCTGGTCCCACGGTTTCATGC |
| | CGACTTCATTCATAAGCACGTGAAAGGCTCACGGTTTGGATGGCGTCAGAAGGAATGC |
| | CTGAAGAAGTGATATGCCATGTTGCTGCCCAGTTTCACACTGGAAGAGATCCTGTGCA |
| | AAGATCCAGCGGCCTGCTTTGGGTTCCAGTAAACACAAAAGCTGCATTTGATGCCAGA |
| | AGGCAAACACAACCTGCATTTGCGTTTTGCAGATGAATTCAACAAGTTAGCAGAAGAC |
| | TTCCTACAATGAGAATGCACACTCC |
| | ORF Start: ATG at 64 ORF Stop: TAA at 607 |
| | SEQ ID NO: 112 181 aa MW at 20115.7kD |
| NOV34b, | MPRNLLYSLLSSHLSPHFSTSVTSAKVAVNGVQLHYQQTGEGDHAVLLLPGMLGSGET |
| CG131881-03 | |
| Protein | DFGPQLKNLNKKLFTVVAWDPRGYGHSRPPDRDFPADFFERDAKDAVDLMKALKFKKV |
| Sequence | |
| | SLLGWSDGGITALTAAAKYPSYTHKMVIWGANAYVTDEDSMIYEGIRDVSKWSERTRK |
| | PLEALYG |
| | SEQ ID NO: 113 828 bp |
| NOV34c, | GGAACTGAAAATTCAGAATCCTGGGCCTCACTCCCAGAGGATCTGATCTACATGTGTG |
| CG131881-04 | |
| DNA | GAGATGCCCAGGAATCTGCTTTATTCTCTTTTGTCCTCCCACCTGTCCCCCCATTTCG |
| Sequence | |
| | GCACCTCGGTAACCTCTGCCAAAGTGGCTGTGAATGGCGTTCAGCTGCATTACCAGCA |
| | GACTGGAGAGGGAGATCACGCAGTCCTGCTACTTCCTGGGATGTTAGGAAGTGGAGAG |
| | ACTGATTTTGGACCTCAGCTCAAGAACCTCAATAAGAAGCTCTTCACAGTGGTCGCCT |
| | GGGATCCTCGAGGCTATGGACATTCCAGGCCCCCAGATCGCGATTTCCCAGCAGACTT |
| | TTTTGAkAGGGATGCAAAAGATGCTGTTGATTTGATGAAGGCGCTGAAGTTTAAGAAG |
| | GTTTCTCTGCTGGGGTGGAGTGATGGGGGCATAACCGCACTCATTGCTGCTGCAAAAT |
| | ATCCATCTTACATCCACAAGATGGTGATCTGGGGCGCCAACGCCTACGTCACTGACGA |
| | AGACAGCATGATATATGAGGGCATCCGAGATGTTTCCATGGAGTGAGAGAAAACAAGA |
| | AAGCCTCTAGAAGCCCTCTATGGGTAACATCTGCCGGCACCTGCTGCCCCGGGTCCAG |
| | TGCCCCGCCTTGATTGTGCACGGTGAGGAGGATCCTCTGGTCCCACGGTTTCATGCCG |
| | ACTTCATTCATAAGCACGTGAAAGGCTCACGGCTGCATTTGATGCCAGAAGGCAAACA |
| | CAACCTGCATTTGCGTTTTGCAGATGAATTCAACAAGTTAGCAGAAGACTTCCTACAA |
| | TGAGAATGCACACTCC |
| | ORF Start: ATG at 62 ORF Stop: TAA at 605 |
| | SEQ ID NO: 114 181 aa MW at 20085.7kD |
| NOV34c | MPRNLLYSLLSSHLSPHFGTSVTSAKVAVNGVQLHYQQTGEGDHAVLLLPGMLGSGET |
| CG131881-04 | |
| Protein | DFGPQLKNLNKKLFTVVAWDPRGYGHSRPPDRDPPADFFERDAKDAVDLMKALKFKKV |
| Sequence | |
| | SLLGWSDGGITALIAAAKYPSYIHKMVIWGANAYVTDEDSMIYEGTRDVSKWSERTRK |
| | PLEALYG |
| | SEQ ID NO: 115 1028 bp |
| NOV34d, | GGAACTGAAAATTCAGAATCCTGGGCCTCACTCCCAGAGGATCTGATCTACATGTGTG |
| CG131881-05 | |
| DNA | GAGATGCCCAGGAATCTGCTTTATTCTCTTTTGTCCTCCCACCTGTCCCCCCATTTCA |
| Sequence | |
| | GCACCTCGGTAACCTCTGCCAAAGTGGCTGTGAATGGCGTTCAGCTGCATTACCAGCA |
| | GACTGGAGAGGGAGATCACGCAGTCCTGCTACTTCCTGGGATGTTAGGAAGTGGAGAG |
| | ACTGATTTTGGACCTCAGCTCAAGAACCTCAATAAGAAGCTCTTCACGGTGGTCGCCT |
| | GGGATCCTCGAGGCTATGGACATTCCAGGCCCCCAGATCGCGATTTCCCAGCAGACTT |
| | TTTTGAAAGGGATGCAAAAGATGCTGTTGATTTGATGAAGGCGCTGAAGTTTAAGAAG |
| | GTTTCTCTGCTGGGGTGGAGTGATGGGGGCATAACCGCACTCATTGCTGCTGCAAAAT |
| | ATCCATCTTACATCCACAAGATGGTGATCTGGGGCGCCAACGCCTACGTCACTGACGA |
| | AGACAGCATGATATATGAGGGCATCCGAGATGTTTCCAAATGGAGTGAGAGAACAAGA |
| | AAGCCTCTAGAAGCCCTCTATGGGTAACATCTGCCGGCACCTGCTGCCCCGGGTCCAG |
| | TGCCCCGCCTTGATTGTGCACGGTGAGAAGGATCCTCTGGTCCCACGGTTTCATGTCG |
| | ACTTCATTCATAAGCACGTGAAAGGCTCACGGTGGGGCTTTCTAGAAGAAGCAGAATG |
| | AAAAAGGAAAATATTTAGTTTCTGAATAAAAAGGGGCTATTGGCAACCAGGTTTGGAT |
| | GGCGTCAGAAGGAATGCCTGAAGAAGTGATATGCCATGTTGCTGCCCAGTTTCACACT |
| | GGAAGAGATCCTGTGCAAAGATCCAGCGGCCTGCTTTGGGTTCCAGTAAACACAAAAG |
| | CTGCATTTGATGCCAGAAGGCAAACACAACCTGCATTTGCGTTTTGCAGATGAATTCA |
| | ACAAGTTAGCAGAAGACTTCCTACAATGAGAATGCACACTCC |
| | ORF Start: ATG at 62 ORF Stop: TAA at 605 |
| | SEQ ID NO: 116 181 aa MW at 20115.7kD |
| NOV34d, | MPRNLLYSLLSSHLSPHFSTSVTSAKVAVNGVQLHYQQTGEGDHAVLLLPGMLGSGET |
| CG131881-05 | |
| Protein | DFGPQLKNLNKKLFTVVAWDPRGYGHSRPPDRDFPADFFERDAKDAVDLMKALKFKKV |
| Sequence | |
| | SLLGWSDGGTTALIAAAKYPSYTHKMVTWGANAYVTDEDSMIYEGIRDVSKWSERTRK |
| | PLEALYG |

Sequence comparison of the above protein sequences yields the following sequence relationships shown in Table 34B.

TABLE 34B
Comparison of NOV34a against NOV34b through NOV34d

| | | Identities/ |
|---|---|---|
| Protein | NOV34a Residues/ | Similarities for |
| Sequence | Match Residues | the Matched Region |
| NOV34b | 1 . . . 161 | 144/161 (89%) |
| | 1 . . . 161 | 144/161 (89%) |
| NOV34c | 1 . . . 161 | 149/161 (92%) |
| | 1 . . . 161 | 149/161 (92%) |
| NOV34d | 1 . . . 161 | 144/161 (89%) |
| | 1 . . . 161 | 144/161 (89%) |

Further analysis of the NOV34a protein yielded the following properties shown in Table 34C.

TABLE 34C
Protein Sequence Properties NOV34a

| | |
|---|---|
| PSort | 0.7403 probability located in microbody (peroxisome); |
| analysis: | 0.2112 probability located in lysosome (lumen); 0.1000 |
| | probability located in mitochondrial matrix space; 0.0000 |
| | probability located in endoplasmic reticulum (membrane) |
| SignalP | No Known Signal Sequence Predicted |
| analysis: | |

A search of the NOV34a protein against the Geneseq database, a proprietary database that contains sequences published in patents and patent publication, yielded several homologous proteins shown in Table 34D.

TABLE 34D
Geneseq Results for NOV34a

| | | NOV34a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| ABG22318 | Novel human diagnostic protein #22309- | 1 . . . 230 | 222/279 (79%) | e−122 |
| | Homo sapiens, 284 aa. | 6 . . . 284 | 226/279 (80%) | |
| | [WO200175067-A2, 11 Oct. 2001] | | | |
| ABG22318 | Novel human diagnostic protein #22309- | 1 . . . 230 | 222/279 (79%) | e−122 |
| | Homo sapiens, 284 aa. | 6 . . . 284 | 226/279 (80%) | |
| | [WO200175067-A2, 11 Oct. 2001] | | | |
| ABB61473 | Drosophila melanogaster polypeptide | 23 . . . 228 | 97/252 (38%) | 2e−47 |
| | SEQ ID NO 11211-Drosophila | 22 . . . 273 | 141/252 (55%) | |
| | melanogaster, 278 aa. [WO200171042- | | | |
| | A2, 27 Sep. 2001] | | | |
| AAW00549 | Protein sequence of BA 70.1 fragment- | 160 . . . 219 | 58/60 (96%) | 5e−30 |
| | Homo sapiens, 99 aa. | 40 . . . 99 | 59/60 (97%) | |
| | [U.S. Pat. No. 5536647-A, 16 Jul. 1996] | | | |
| AAU34331 | Staphylococcus aureus cellular | 21 . . . 142 | 45/132 (34%) | 2e−09 |
| | proliferation protein #607- | 1 . . . 26 | 66/132 (49%) | |
| | Staphylococcus aureus, 241 aa. | | | |
| | [WO200170955-A2, 27 Sep. 2001] | | | |

In a BLAST search of public sequence datbases, the NOV34a protein was found to have homology to the proteins shown in the BLASTP data in Table 34E.

TABLE 34E
Public BLASTP Results for NOV34a

| | | NOV34a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q13855 | Biphenyl hydrolase-related protein- | 1 . . . 230 | 230/274 (83%) | e−129 |
| | Homo sapiens (Human), 274 aa. | 1 . . . 274 | 230/274 (83%) | |
| Q8R164 | Similar to RIKEN cDNA | 18 . . . 230 | 186/257 (72%) | e−104 |
| | 2010012D11 gene-Mus musculus | 35 . . . 291 | 201/257 (77%) | |
| | (Mouse), 291 aa. | | | |
| Q8R589 | Similar to RIKEN cDNA | 18 . . . 230 | 185/257 (71%) | e−103 |
| | 2010012D11 gene-Mus musculus | 35 . . . 291 | 200/257 (76%) | |
| | (Mouse), 291 aa. | | | |
| Q9DCC6 | 2010012D11Rik protein-Mus | 18 . . . 230 | 185/257 (71%) | e−103 |
| | musculus (Mouse), 291 aa. | 35 . . . 291 | 200/257 (76%) | |
| Q9CYD0 | 5730533B08Rik protein-Mus | 18 . . . 161 | 123/144 (85%) | 1e−69 |
| | musculus (Mouse), 245 aa. | 43 . . . 186 | 136/144 (94%) | |

PFam analysis predicts that the NOV34a protein contains the domains shown in the Table 34F.

TABLE 34F
Domain Analysis of NOV34a

| | | Identities/ | |
|---|---|---|---|
| Pfam | NOV34a | Similarities for | Expect |
| Domain | Match Region | the Matched Region | Value |
| DLH | 167 . . . 200 | 12/34 (35%) | 0.26 |
| | | 29/34 (85%) | |
| abhydrolase | 72 . . . 229 | 46/235 (20%) | 0.0097 |
| | | 120/235 (51%) | |

### Example 35

The NOV35 clone was analyzed, and the nucleotide and encoded polypeptide sequences are shown in Table 35A.

TABLE 35A
NOV35 Sequence Analysis

| | |
|---|---|
| | SEQ ID NO: 117 1218 bp |
| NOV35a, | GCGGCGGCTGCCCGGCGGCCCGGGCGCGCGGCGCTTCGCCATGTACACCTTCGTCGTA |
| CG133535-01 | |
| DNA | CGCGATGAGAACAGCAGCGTCTACGCCGAGGTCTCCCGGCTGCTCCTCGCCACCGGCC |
| Sequence | |
| | ACTGGAAGAGGCTGCGGCGAGACAACCCCAGATTCAACCTGATGCTGGGAGAGAGGAA |
| | TCGGCTGCCCTTCGGGAGACTGGGTCACGAGCCCGGGCTGGTACAGTTGGTGAATTAC |
| | TACAGGGGTGCTGACAAACTGTGTCGCAAAGCTTCTTTAGTGAAGCTAATCAAGACAA |
| | GCCCTGAACTGGCTGAGTCCTGCACATGGTTCCCTGAATCTTATGTGATTTATCCAAC |
| | CAATCTCAAGACTCCAGTTGCTCCAGCACAGAATGGAATTCAGCCACCAATCAGTAAC |
| | TCAAGGACAGATGAAAGAGAATTCTTTCTCGCCTCTTATAACAGAAAGAAAGAGGATG |
| | GAGAGGGCAACGTTTGGATTGCAAAGTCATCAGCCGGTGCCAAAGGTGAGGGCATTCT |
| | CATCTCCTCAGAGGCTTCAGAGCTTCTCGATTTCATAGACAACCAGGGCCAAGTGCAC |
| | GTGATCCAGAAATATCTTGAGCACCCTCTGCTGCTTGAGCCAGGTCATCGCAAGTTTG |
| | ACATTCGAAGCTGGGTCTTGGTGGATCATCAGTATAATATCTACCTCTATAGAGAGGG |
| | TGTGCTTCGGACTGCTTCAGAACCATATCATGTTGATAATTTCCAAGACAAAACCTGC |
| | CATTTGACCAATCACTGCATTCAAAAAGAGTATTCAAAGAACTACGGGAAGTATGAAG |
| | AAGGAAATGAAATGTTCTTCAAGGAGTTCAATCAGTACCTAACAAGTGCTTTGAACAT |
| | TACCCTAGAAAGTAGTATCTTACTACAAATCAAACATATAATCAGGAACTGCCTCCTG |
| | AGCGTGGAGCCTGCCATTAGCACCAAGCACCTCCCTTACCAGAGCTTCCAGCTCTTCG |
| | GCTTTGACTTCATGGTCGATGAGGAGCTGAAGGTGTGGCTCATTGAGGTCAACGGTGC |
| | CCCTGCATGTGCTCAGAAGCTCTATGCAGAACTGTGCCAAGGCATCGTGGACATAGCC |
| | ATTTCCAGTGTCTTCCCACCCCCAGATGTGGAGCAACCTCAGACCCAGCCAGCTGCCT |
| | TCATCAAGCTGTGACAGAGGGCACTCCCTGCTGCCTTGGAAAAAGCACGGGGTCCTGC |
| | ORF Start: ATG at 41 ORF Stop: TGA at 1172 |
| | SEQ ID NO: 118 377 aa MW at 43211.8kD |
| NOV35a, | MYTFVVRDENSSVYAEVSRLLLATGHWKRLRRDNPRFNLMLGERNRLPFGRLGHEPGL |
| CG133535-01 | |
| Protein | VQLVNYYRGADKLCRKASLVKLIKTSPELAESCTWFPESYVIYPTNLKTPVARAQNGI |
| Sequence | |
| | QPPISNSRTDEREFFLASYNRKKEDGEGNVWIAKSSAGAKGFGILISSEASELLDFID |
| | NQGQVHVIQKYLEHPLLLFRGHRKFDIRSWVLVDHQYNIYLYREGVLRTASEPYHVDN |
| | FQDKTCHLTNHCTQKEYSKNYGKYEEGNEMFFKEFNQYLTSALNITLFSSILLQIKHI |
| | IRNCLLSVEPATSTKHLPYQSFQLFGFDFMVDEELKVWLIEVNGAPACAQKLYAELCQ |
| | GIVDIAISSVFPPPDVEQPQTQRAAFIKL |

Further analysis of the NOV35a protein yielded the following properties shown in Table 35B.

TABLE 35B
Protein Sequence Properties NOV35a

| | |
|---|---|
| PSort | 0.4641 probability located in mitochondrial matrix space; |
| analysis: | 0.3581 probability located in microbody (peroxisome); 0.1627 |
| | probability located in mitochondrial inner membrane; 0.1627 |
| | probability located in mitochondrial intermembrane space |
| SignalP | No Known Signal Sequence Predicted |
| analysis: | |

A search of the NOV35a protein against the Geneseq database, a proprietary database that contains sequences published in patents and patent publication, yielded several homologous proteins shown in Table 35C.

TABLE 35B
Geneseq Results for NOV35a

| | Protein/Organism/ | NOV35a | Identities/ | |
|---|---|---|---|---|
| Geneseq | Length [Patent #, | Residues/ | Similarities for | Expect |
| Identifier | Date] | Residues | the Matched Region | Value |
| AAM79068 | Human protein SEQ ID NO 1730- | 5 . . . 377 | 373/373 (100%) | 0.0 |
| | Homo sapiens, 377 aa. | 5 . . . 377 | 373/373 (100%) | |
| | [WO200157190-A2, 9 Aug. 2001] | | | |
| AAM80052 | Human protein SEQ ID NO 3698- | 5 . . . 156 | 152/152 (100%) | 2e−85 |
| | Homo sapiens, 190 aa. | 9 . . . 160 | 152/152 (100%) | |
| | [WO200157190-A2, 9 Aug. 2001] | | | |
| ABG12642 | Novel human diagnostic protein | 106 . . . 251 | 136/146 (93%) | 8e−77 |
| | #12633-Homo sapiens, 146 aa. | 1 . . . 146 | 140/146 (95%) | |
| | [WO200175067-A2, 11 Oct. 2001] | | | |
| ABG12642 | Novel human diagnostic protein | 106 . . . 251 | 136/146 (93%) | 8e−77 |
| | #12633-Homo sapiens, 146 aa. | 1 . . . 146 | 140/146 (95%) | |
| | [WO200175067-A2, 11 Oct. 2001] | | | |
| ABG09620 | Novel human diagnostic protein | 218 . . . 347 | 117/130 (90%) | 2e−63 |
| | #9611-Homo sapiens, 185 aa. | 1. . . 130 | 117/130 (90%) | |
| | [WO200175067-A2, 11 Oct. 2001] | | | |

In a BLAST search of public sequence datbases, the NOV35a protein was found to have homology to the proteins shown in the BLASTP data in Table 35D.

TABLE 35D
Public BLASTP Results for NOV35a

| | | NOV35a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q8VEG2 | Hypothetical 43.1 kDa protein-Mus | 1 . . . 377 | 359/377 (95%) | 0.0 |
| | musculus (Mouse), 377 aa. | 1 . . . 377 | 369/377 (97%) | |
| P38160 | Tubulin-tyrosine ligase (EC | 1 . . . 377 | 360/379 (94%) | 0.0 |
| | 6.3.2.25) (TTL)-Sus scrofa (Pig), | 1 . . . 379 | 369/379 (96%) | |
| | 379 aa. | | | |
| QSR11.7 | Hypothetical 43.1 kDa protein-Mus | 1 . . . 377 | 358/377 (94%) | 0.0 |
| | musculus (Mouse), 377 aa. | 1 . . . 377 | 368/377 (96%) | |
| Q9QXJ0 | Tubulin-tyrosine ligase (EC | 1 . . . 377 | 357/377 (94%) | 0.0 |
| | 6.3.2.25) (TTL)-Rattus norvegicus | 1 . . . 377 | 368/377 (96%) | |
| | (Rat), 377 aa. | | | |
| P38584 | Tubulin-tyrosine ligase (EC | 1 . . . 377 | 354/377 (93%) | 0.0 |
| | 6.3.2.25) (TTL)-Bos taurus | 1 . . . 377 | 368/377 (96%) | |
| | (Bovine), 377 aa. | | | |

PFam analysis predicts that the NOV35a protein contains the domains shown in the Table 35E.

TABLE 35E
Domain Analysis of NOV35a

| Pfam | NOV35a | for the | Expect |
|---|---|---|---|
| Domain | Match Region | Matched Region | Value |
| TTL | 81 . . . 367 | 108/334 (32%) | 2.1e−108 |
| | | 254/334 (76%) | |

### Example 36

The NOV36 clone was analyzed, and the nucleotide and encoded polypeptide sequences are shown in Table 36A.

TABLE 36A
NOV36 Sequence Analysis

| | |
|---|---|
| | SEQ ID NO: 119 4562 bp |
| NOV36a, | AGAGGAGACTTGATCTCTAGTTCATTCTGGAACTCCGCCTGGGATTGTGCACTGTCCA |
| CG133558-01 | |
| DNA | GGGTCCTGAAACATGAACCAAACTGCCAGCGTGTCCCATCACATCAAGTGTCAACCCT |
| Sequence | |
| | CAAAAACAATCAAGGAACTGGGAAGTAACAGCCCTCCACAGAGAAACTGGAAGGGAAT |
| | TGCTATTGCTCTGCTGGTGATTTTAGTTGTATGCTCACTCATCACTATGTCAGTCATC |
| | CTCTTAACCCCAGGTTTAGATGAACTCACAAATTCGTCAGAAACCAGATTGTCTTTGG |
| | AAGACCTCTTTAGGAAAGACTTTGTGCTTCACGATCCAGAGGCTCGGTGGATCAATGG |
| | TAAGGATGTGGTGTATAAAAGCGAGAATGGACATGTCATTAAACTGAATATAGAAACA |
| | AATGCTACCACATTATTATTGGAAAACACAACTTTTGTAACCTTCAAAGCATCAAGAC |
| | ATTCAGTTTCACCAGATTTAAAATATGTCCTTCTGGCATATGATGTCAAACAGATTTT |
| | TCATTATTCGTATACTGCTTCATATGTGATTTACAACATACACACTAGGGAAGTTTGG |
| | GAGTTAAATCCTCCAGAAGTAGAGGACTCCGTCTTGCAGTACGCGGCCTGGGGTGTCC |
| | AAGGGCAGCAGCTGATTTATATTTTTGAAAATAATATCTACTATCAACCTGATATAAA |
| | GAGCAGTTCATTGCGACTGACATCTTCTGGAAAAGAAGAAATAATTTTTAATGGGATT |
| | GCTGACTGGTTATATGAAGAGGAACTCCTGCATTCTCACATCGCCCACTGGTGGTCAC |
| | CAGATGGAGAAAGACTTGCCTTCCTGATGATAAATGACTCTTTGGTACCCACCATGGT |
| | TATCCCTCGGTTTACTGGAGCGTTGTATCCCAAAGGAAAGCAGTATCCGTATCCTAAG |
| | GCAGGTCAAGTGAACCCAACAATAAAATTATATGTTGTAAACCTGTATGGACCAACTC |
| | ACACTTTGGAGCTCATGCCACCTGACAGCTTTAAAATCAAGAGATACTATATCACTAT |
| | GGTTAAATGGGTAAGCAATACCAAGACTGTGGTAAGATGGTTAAACCGACCTCAGAAC |
| | ATCTCCATCCTCACAGTCTGTGAGACCACTACAGGTGCTTGTAGTAAAAAATATGAGA |
| | TGACATCAGATACGTGGCTCTCTCAGCAGAATGAGGAGCCCGTGTTTTCTAGAGACGG |
| | CAGCAAATTCTTTATGACAGTGCCTGTTAAGCAAGGGGGACGTGGAGAATTTCACCAC |
| | ATAGCTATGTTCCTCATCCAGAGTAAAAGTGAGCAAATTACCGTGCGGCATCTGACAT |
| | CAGGAAACTGGGAAGTGATAAAGATCTTGGCATACGATGAAACTACTCAAAAAATTTA |
| | CTTTCTGAGCACTGAATCTTCTCCCAGAGGAAGGCAGCTGTACAGTGCTTCTACTGAA |
| | GGATTATTGAATCGCCAATGCATTTCATGTAATTTCATGAAAGAACAATGTACATATT |
| | TTGATGCCAGTTTTAGTCCCATGAATCAACATTTCTTATTATTCTGTGAAGGTCCAAG |
| | GGTCCCAGTGGTCAGCCTACATAGTACGGACAACCCAGCAAAATATTTTATATTGGAA |
| | AGCAATTCTATGCTGAAGGAAGCTATCCTGAAGAAGAAGATAGGAAAGCCAGAAATTA |
| | AAATCCTTCATATTGACGACTATGAACTTCCTTTACAGTTGTCCCTTCCCAAAGATTT |
| | TATGGACCGAAACCAGTATGCTCTTCTGTTAATAATGGATGAAGAACCAGGAGGCCAG |
| | CTGGTTACAGATAAGTTCCATATTGACTGGGATTCCGTACTCATTGACATGGATAATG |
| | TCATTGTAGCAAGATTTGATGGCAGAGGAAGTGGATTCCAGGGTCTGAAAATTTTGCA |
| | GGAGATTCATCCAAGATTAGGTTCAGTAGAAGTAAAGGACCAAATAACAGCTGTGAAA |
| | TTTTTGCTGAAACTGCCTTACATTGACTCCAAAAGATTAAGCATTTTTGGAAAGGGTT |
| | ATGGTGGCTATATTGCATCAATGATCTTAAAATCAGATGAAAAGCTTTTTAAATGTGG |
| | ATCCGTGGTTGCACCTATCACAGACTTGAAATTGTATGCCTCAGCTTTCTCTGAAAGA |
| | TACCTTGGGATGCCATCTAAGGAAGAAAGCACTTACCAGGCAGCCAGTGTGCTACATA |
| | ATGTTCATGGCTTGAAAGAAGAAAATATATTAATAATTCATGGAACTGCTGACACAAA |
| | AGTTCATTTCCAACACTCAGCAGAATTAATCAAGCACCTAATAAAGCTGGAAGTGAAT |
| | TATACTATGCAGGTCTACCCAGATGAAGGTCATAACGTATCTGAGAAGAGCAAGTATC |
| | ATCTCTACAGCACAATCCTCAAATTCTTCAGTGATTGTTTGAAGGAAGAAATATCTGT |
| | GCTACCACAGGAACCAGAAGAGATGAATAATGGACCGTATTTATACAGAACTGAAAGG |
| | GAATATTGAGGCTCAATGAAACCTGACAAAGAGACTGTAATATTGTAGTTGCTCCAGA |
| | ATGTCAAGGGCAGCTTACCGAGATGTCACTGGAGCAGCACGCTCAGAGACAGTGAACT |
| | AGCATTTGAATACACAAGTCCAAGTCTACTGTGTTGCTAGGGGTGCAGAACCCGTTTC |
| | TTTGTATGAGAGAGGTCAAGGGTTGGTTTCCTGGGAGAAAAATTAGTTTTGCATTAAG |
| | TAGGAGTAGTGCATGTTTTCTTCTGTTATCCCCCTGTTTGTTCTGTAACTAGTTGCTC |
| | TCATTTTAATTTCACTGGCCACCATCATCTTTGCATATAATGCACAATCTATCATCTG |
| | TCCTACAGTCCCTGATCTTTCATGGCTGAGCTGCAATCTAACACTTTACTGTACCTTT |
| | ATAATAAGTGCAATTCTTTCATTGTCTATTATTGTGCTTAAGAAAATATTCAGTTAAT |
| | AAAAAACAGAGTATTTTATGTAATTTCTGTTTTTAAAAAGACATTATTAAATGGGTCA |
| | AGGACATATAGAAAGTGTGGATTTCAGCACCTTCCAAAGTTCAGCCAGTTATCAGTAG |
| | ATACAATATCTTTAATGAACACACGAGTGTATGTCTCACAATATATATACACAAAGTG |
| | TGCATATACAGTTAATGAAACTATCTTTAAATGTTATTCATGCTATAAAGAGTAAACG |
| | TTTGATGAATTAGAAGAGATGCTCTTTTCCAAGCTATAATGGATGCTTTGTTTAATGA |
| | GCCAAATATGATGAAACATTTTTTCCAATTCAAATTCTAGCTATTGCTTTCCTATAAA |
| | TGTTTGGGTTGTGTTTGGTATTGTTTTTAGTGGTTAATAGTTTTCCAGTTGCATTTAA |
| | TTTTTTGAATATGATACCTTGTCACATGTAAATTAGATACTTAAATATTAAATTATAG |
| | TTTCTGATAAAGAAATTTTGTTAACAATGCAATGCCACTGAGTGCTATTTTGCTCTTT |
| | TGGTGGAGAAGGCTTTTTTCAAAACTCTTGGTCCTTTTACTTCTTTCTCTCAGTGCAG |
| | AATCAATTCTCATTTTCATCGTAAAAGCAAATAGCTGGATTATTTCATTTGCCAGTTT |
| | CTATTTAGTATTCCATGCCTGCCCAATTCATCTGTTACTGTTTAATTTCAATTCTTCT |
| | GGTGAGAATTAGAAATGAAATATTTTTTATTCATTGGCCAAAAAGTTCACAGACAGCA |
| | GTGTTTGCTATTTACTTTGAATTGAAGGCACAAAATGCATCAATTCCTGTGCTGTGTT |
| | GACTTGCAGTAGTAAGTAACTGAGAGCATAAAATAAACCTGACTGTATGAAGTCAATT |
| | TAAGTGATGAGAACATTTAACTTTGGTGACTAAAGTCAGAATATCTTCTCACTTCACT |
| | TAAGGGATCTTCCAGAAGATATCTAAAAGTCTGTAATAAGCTTAGAAGTTCAGATAAA |
| | TCTAGGCAGGATACTGCATTTTTGTGGTTTTAAAAAAGTCCTTAGGACAGACTGAATT |
| | ATCATAACTTATGGCATCAGGAGGAAACTTTAAAATATCAAGGAATCACTCAGTCACC |
| | CTCCTGTTTTGTTGAAGGATCAACCCCAAATTCTGGGTATTTGAGTACATGTGAATCA |
| | TGGATTTGGTATTCAACTTTTTCCCTGGATGCTTTGGAATCGTGTCTTCCATGCTCCA |
| | TTGGGTTCAATTTAAAATAGGAGAGGCTTTCTCTTCTGAAAGATCCATTTTAGGTCTT |
| | TTTCAAGAATAGTGAACACATTTTTTAACAAAATAAGTTGTAATTTTAAAAGGAAAGT |
| | TTTGCCTATTTTATTAAGATGGAAATTTCTTTTTAGGCTAATTTGAAATCCAACTGAA |
| | GCTTTTTAACCAATATTTTAAATTTGAACCACTAGAGTTTTTTATGATGCAAATGATT |
| | ATGTTGTCTGAAAGGTGTGGTTTTATTGAATGTCTATTTGAGTATCATTTAAAAAGTA |
| | TTTGCCTTTTACTGTCATCATTTCTCTTGTTTTATTATTATTATCAATGTTTATCTAT |
| | TTTTCAATTAATTTAATACAGTTTCTAATGTGAAAGAC |
| | ORF Start: ATG at 71 ORF Stop: TAA at 2465 |
| | SEQ ID NO: 120 1798 aa MW at 91066.5kD |
| NOV36a, | MNQTASVSHHTKCQPSKTTKELGSNSPPQRNWKGIAIALLVTLVVCSLTTMSVTLLTP |
| CG133558-01 | |
| Protein | GLDELTNSSETRLSLEDLPRKDPVLHDPEARWINGKDVVYKSENGHVIKLNTETNATT |
| Sequence | |
| | LLLENTTFVTFKASRHSVSRDLKYVLLAYDVKQIFHYSYTASYVIYNTHTREVWELNP |
| | PEVEDSVLQYAAWGVQGQQLIYIFENNIYYQPDTKSSSLRLTSSGKEEIIFNGIADWL |
| | YEEELLHSHIAHWWSPDGERLAFLMINDSLVPTMVIPRFTGALYPKGKQYPYPKAGQV |
| | NPTTKLYVVNLYGPTHTLELMPPDSFKSREYYITMVKWVSNTKTVVRWLNRPQNTSIL |
| | TVCETTTGACSKKYEMTSDTWLSQQNEEPVFSRDGSKFFMTVPVKQGGRGEFHHIAMF |
| | LIQSKSFQITVRHLTSGNWEVTKILAYDETTQKTYFLSTESSPRGRQLYSASTEGLLN |
| | RQCISCNFMKEQCTYFDASFSPMNQHFLLPCEGPRVPVVSLHSTDNPAKYFILESNSM |
| | LKEAILKKKIGKPEIKTLHIDDYELPLQLSLPKDFMDRNQYALLLIMDEEPGGQLVTD |
| | KFHIDWDSVLIDMDNVIVARFDGRGSGFQGLKILQEIHRRLGSVEVKDQITAVKFLLK |
| | LPYTDSKRLSIFGKGYGGYIASMILKSDEKLFKCGSVVAPITDLKLYASAFSERYLGM |
| | PSKEESTYQAASVLHNVHGLKEENILIIHGTADTKVHFQHSAELTKHLIKAGVNYTMQ |
| | VYPDEGHNVSEKSKYHLYSTTLKFFSDCLKEEISVLPQEPEEDE |

Further analysis of the NOV36a protein yielded the following properties shown in Table 36B.

TABLE 36B
Protein Sequence Properties NOV36a

| | |
|---|---|
| PSort | 0.7900 probability located in plasma membrane; 0.3000 |
| analysis: | probability located in Golgi body; 0.2426 probability |
| | located in microbody (peroxisome); 0.2000 probability |
| | located in endoplasmic reticulum (membrane) |
| SignalP | Cleavage site between residues 53 and 54 |
| analysis: | |

A search of the NOV36a protein against the Geneseq database, a proprietary database that contains sequences published in patents and patent publication, yielded several homologous proteins shown in Table 36C.

TABLE 36C
Geneseq Results for NOV36a

| | | NOV36a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| ABB04588 | Human aminopeptidase 21956-Homo | 1 . . . 798 | 794/798 (99%) | 0.0 |
| | sapiens, 796 aa. [WO200192493-A2 | 1 . . . 796 | 794/798 (99%) | |
| | 6 Dec. 2001] | | | |
| AAB11748 | Rat dipeptidyl peptidase IV (DPPIV)- | 32 . . . 782 | 271/773 (35%) | e−138 |
| | Rattus sp. 767 aa. [JP2000143699-A, | 5 . . . 763 | 442/773 (57%) | |
| | 6 May 2000] | | | |
| ABB08991 | Human dipeptidyl peptidase IV-Homo | 32 . . . 782 | 267/773 (34%) | e−136 |
| | sapiens, 766 aa. | 5 . . . 762 | 439/773 (56%) | |
| | [U.S. Pat. No. 6337069-B1, | | | |
| | 8 Jan. 2002] | | | |
| AAG78417 | Human dipeptidyl peptidase IV amino | 32 . . . 782 | 267/773 (34%) | e−136 |
| | acid sequence-Homo sapiens, 766 aa. | 5 . . . 762 | 439/773 (56%) | |
| | [WO200179473-A2, 25 Oct. 2001] | | | |
| AAR40909 | Sequence encoded by human CD26 | 32 . . . 782 | 267/773 (34%) | e−136 |
| | cDNA-Homo sapiens, 766 aa. | 5 . . . 762 | 439/773 (56%) | |
| | [WO9316102-A, 19 Aug. 1993] | | | |

In a BLAST search of public sequence datbases, the NOV36a protein was found to have homology to the proteins shown in the BLASTP data in Table 36D.

TABLE 36D
Public BLASTP Results for NOV36a

| | | NOV36a | | |
|---|---|---|---|---|
| Protein | | Residues/ | Identities/ | |
| Accession | | Match | Similarities for | Expect |
| Number | Protein/Organism/Length | Residues | the Matched Portion | Value |
| CAD20410 | Sequence 1 from Patent WO0192493- | 1 . . . 798 | 794/798 (99%) | 0.0 |
| | Homo sapiens (Human), 796 aa. | 1 . . . 796 | 794/798 (99%) | |
| Q9P236 | KIAA1492 protein-Homo sapiens | 88 . . . 798 | 709/711 (99%) | 0.0 |
| | (Human), 711 aa (fragment). | 1 . . . 711 | 709/711 (99%) | |
| Q9Z218 | Dipeptidyl peptidase IV like protein | 1 . . . 797 | 414/806 (51%) | 0.0 |
| | (Dipeptidyl aminopeptidase-related | 1 . . . 804 | 567/806 (69%) | |
| | protein) (Dipeptidylpeptidase VI) (DPPX) | | | |
| | (Dipeptidylpeptidase 6) (Dipeptidyl | | | |
| | peptidase-like protein 6)-Mus musculus | | | |
| | (Mouse), 804 aa. | | | |
| I68600 | dipeptidyl aminopeptidase like protein- | 20 . . . 798 | 411/784 (52%) | 0.0 |
| | human, 803 aa. | 19 . . . 800 | 555/784 (70%) | |
| P42658 | Dipeptidyl peptidase IV like protein | 21 . . . 798 | 411/783 (52%) | 0.0 |
| | (Dipeptidyl aminopeptidase-related | 82 . . . 862 | 554/783 (70%) | |
| | protein) (Dipeptidylpeptidase VI) (DPPX)- | | | |
| | Homo sapiens (Human), 865 aa. | | | |

PFam analysis predicts that the NOV36a protein contains the domains shown in the Table 36E.

TABLE 36E
Domain Analysis of NOV36a

| | | Identities/ | |
|---|---|---|---|
| Pfam | NOV36a | Similarities for | Expect |
| Domain | Match Region | the Matched Region | Value |
| DPPIV_N_term | 71 . . . 580 | 199/571 (35%) | 7.1e−73 |
| | | 405/571 (71%) | |
| Peptidase_S9 | 582 . . . 658 | 27/81 (33%) | 6.6e−21 |
| | | 52/81 (64%) | |
| DLH | 721 . . . 761 | 16/41 (39%) | 0.14 |
| | | 33/41 (80%) | |

### Example 37

The NOV37 clone was analyzed, and the nucleotide and encoded polypeptide sequences are shown in Table 37A.

TABLE 37A
NOV37 Sequence Analysis

| | |
|---|---|
| | SEQ ID NO: 121 2057 bp |
| NOV37a, | ATTATATTCCAAATCAGCATGGGCATTAACCATGACAATGACCACCCATCGTGTGCTG |
| CC133589-01 | |
| DNA | ATCGTCTTCATATCATGTCTGGTGAATGGATTAAAGGACAGAATCTTGGTGACGTTTC |
| Sequence | |
| | ATGGTCTCGATGTAGCAAGGAAGATTTGGAAAGATTTCTCAGGTCAAAGGCCAGTAAC |
| | TGCTTGCTACAAACAAATCCGCAGAGTGTCAATTCTGTGATGGTTCCCTCCAAGCTGC |
| | CAGGGATGACATACACTGCTGATGAACAATGCCAGATCCTTTTTGGGCCATTGGCTTC |
| | TTTTTGTCAGGAGATGCAGCATGTTATTTGCACAGGATTATGGTGCAAGGTAGAAGGT |
| | GAGAAAGAATGCAGAACCAAGCTAGACCCACCAATGGATGGAACTGACTGTGACCTTG |
| | GTAAGATTCTGAAGCAAGGGATTGTAATGGTCCCAGAAAACAATACAGAATATGTGAG |
| | AATCCACCTTGTCCTGCAGGTTTGCCTGGATTCAGAGACTGGCAATGTCAGGCTTATA |
| | GTGTTAGAACTTCCTCCCCAAAGCATATACTTCAGTGGCAAGCTGTCCTGGATGAAGT |
| | TGACTCTTAAATACATTAAGGTGGCTGCCCACCCCCATGGTTCTTGGAACACTCGTGT |
| | GCCCTGCTTGGTTGCTGTGTTGTTAACACCTACCCGGCTTTCCTACTACATCTCTGAA |
| | AAACCATGTGCCTTGTTTTGCTCTCCTGTTGGAAAAGAACAGCCTATTCTTCTATCAG |
| | AAAAAGTGATGGATGGAACTTCTTGTGGCTATCAGGGATTAGATATCTGTGCAAATGG |
| | CAGGTGCCAGAAAGTTGGCTGTGATGGTTTATTAGGGTCTCTTGCAAGAGAAGATCAT |
| | TGTGGTGTATGCAATGGCAATGGAAAATCATGCAAGATCATTAAAGGGGATTTTAATC |
| | ACACCAGAGGAGCAGGTTATGTAGAAGTGCTGGTGATACCTGCTGGAGCAAGAAGAAT |
| | CAAAGTTGTGGAGGAAAAGCCGGCACATAGCTATTTAGCTCTCCGAGATGCTGGCAAA |
| | CAGTCTATTAATAGTGACTGGAAGATTGAACACTCTGGAGCCTTCAATTTGGCTGGAA |
| | CTACCGTTCATTATGTAAGACGAGGCCTCTGGGAGAAGATCTCTGCCAAAGGTCCTAC |
| | TACAGCACCTTTACATCTTCTGGTGCTCCTGTTTCAGGATCAGAATTATGGTCTTCAC |
| | TATGAATACACTATCCCATCAGACCCTCTTCCAGAAAACCAGAGCTCTAAAGCACCTG |
| | AGCCCCTCTTCATGTGGACACACACAAGCTGGGAAGATTGCGATGCCACTTGTGGAGG |
| | AGGAGAAAGGAAGACAACAGTGTCCTGCACAAAAATCATGAGCAAAAATATCAGCATT |
| | GTGGACAATGAGAAATGCAAATACTTAACCAAGCCAGAGCCACAGATTCGAAAGTGCA |
| | ATGAGCAACCATGTCAAACAAGGGAATATCTAATAAGTCGTGTGAGTGCTACAAGCCA |
| | GGCAATAGAGAGCAAAGAAAAGGCCAGTCCCCATTGGTTGAATGGAGAAGCCCTTCTA |
| | GGAGGAATGGGCGTGGGGCTGGCTGTCAAGGATCCAGGCACAGGATTCTACAAATATC |
| | ATGAGGTGAAAATAGAAAGTGTTTGGTGGATGATGACAGAATGGACCCCTTGTTCACG |
| | AACTTGTGGAAAAGGAATGCAGAGCAGACAAGTGGCCTGTACCCAACAACTGAGCAAT |
| | GGAACACTGATTAGAGCCCGAGAGAGGGACTGCATTGGGCCCAAGCCCGCCTCTGCCC |
| | AGCGCTGTGAGGGCCAGGACTGCATGACCGTGTGGGAGGCGGGAGTGTGGTCTGAGTG |
| | TTCAGTCAAGTGTGGCAAAGGCATACGTCATCGGACCGTTAGATGTACCAACCCAAGA |
| | AAGAAGTGTGTCCTCTCTACCAGACCCAGGGAGGCTGAAGACTGTGAGGATTATTCAA |
| | AATGCTATGTGTGGCGAATGGGTGACTGGTCTAAGGTGAGAACCATTCTGTATATTCT |
| | CAGTAATAGGTTTCAATAATGTCAGCA |
| | ORF Start: ATG at 19 ORF Stop: TAA at 2047 |
| | SEQ ID NO: 122 676 aa MW at 75430.0kD |
| NOV37a. | MGINHDNDHPSCADGLHIMSGEWIKGQNLGDVSWSRCSKEDLERFLRSKASNCLLQTN |
| CG133389-01 | |
| Protein | PQSVNSVMVPSKLPGMTYTADEQCQILFGPLASFCQEMQHVICTGLWCKVEGEKECRT |
| Sequence | |
| | KLDPPMDGTDCDLGKILKQGIVMVPENNTEYVRTHLVLQVCLDSETGNVPLIVLELPP |
| | QSTYFSGKLSWMKLTLKYTKVAAHRHGSWNTRVRCLVAVLLTPTRLSYYTSEKPCALF |
| | CSPVGKEQPILLSEKVMDGTSCGYQGLDICANGRCQKVGCDGLLGSLAREDHCGVCNG |
| | NGKSCKIIKGDFNHTRGAGYVEVLVIPAGARRIKVVEEKPAHSYLALRDAGKQSINSD |
| | WKIEHSGAPNLAGTTVHYVRRGLWEKISAKGPTTAPLHLLVLLFQDQNYGLHYEYTIP |
| | SDPLPENQSSKAPEPLFMWTHTSWEDCDATCGGGERKTTVSCTKIMSKNTSTVDNEKC |
| | KYLTKPEPQTRKCNEQPCQTREYLISRVSATSQAIESKEKASPHWLNGEALLGGMGVG |
| | LAVKDPGTGFYKYHEVKIESVWWMMTEWTPCSRTCGKGMQSRQVACTQQLSNGTLIRA |
| | RERDCIGPKPASAQRCEGQDCMTVWEAGVWSECSVKCGKGIRHRTVRCTNPRKKCVLS |
| | TRPREAEDCEDYSKCYVWRMGDWSKVRTILYTLSNRFQ |
| | SEQ ID NO 123 3977 bp |
| NOV37b, | GGGAAGAACCGCGAGATGCGCCTGACTCACATCTGCTGCTGCTGCCTCCTTTACCAGC |
| CG133589-02 | |
| DNA | TGGGGTTCCTGTCGAATGGGATCGTTTCAGAGCTGCAGTTCGCCCCCGACCGCGAGGA |
| Sequence | |
| | GTGGGAAGTCGTGTTTCCTGCGCTCTGGCGCCGGGAGCCGGTGGACCCGGCTGGCGGC |
| | AGCGGGGGCAGCGCGGACCCGGGCTGGGTGCGCGGCGTTGGGGGCGGCGGAAGCGCCC |
| | GGGCGCAGGCTGCCGGCAGCTCACGCGAGGTGCGCTCTGTGGCTCCGGTGCCTTTGGA |
| | GGAGCCCGTGGAGGGCCGATCAGAGTCCCGGCTCCGGCCCCCGCCGCCGTCGGAGGGT |
| | GAGGAGGACGAGGAGCTCGAGTCGCAGGAGCTGCCGCGGGGATCCAGCGGGGCTGCCG |
| | CCTTGTCCCCGGGCGCCCCGGCCTCGTGGCAGCCGCCGCCTCCCCCGCAGCCGCCCCC |
| | GTCCCCGCCCCCGGCCCAGCATGCCGAGCCGGATGGCGACGAAGTGTTGCTGCGGATC |
| | CCGGCCTTCTCTCGGGACCTGTACCTGCTGCTCCGGAGAGACGGCCGCTTCCTGGCGC |
| | CGCGCTTCGCAGTGGAACAGCGGCCAAATCCCGGCCCCGGCCCCACGGGGGCAGCATC |
| | CGCCCCGCAACCTCCCGCGCCACCAGACGCAGGCTGCTTCTACACCGGAGCTGTGCTG |
| | CGGCACCCTGGCTCGCTGGCTTCTTTCAGCACCTGTGGAGGTGGCCTGATGGGATTTA |
| | TACAGCTCAATGAGGACTTCATATTTATTGAGCCACTCAATGATACAATGGCCATAAC |
| | AGGTCACCCACACCGTGTATATAGGCAGAAAAGGTCCATGGACGAAAAGGTCACAGAG |
| | AAGTCAGCTCTTCACAGTCATTACTGTGGTATCATTTCAGATAAAGGAAGACCTAGGT |
| | CTAGAAAAATAGCAGAAAGTGGAAGAGGGAAACGATATTCATACAAATTACCTCAAGA |
| | ATACAACATAGAGACTGTAGTGGTTGCAGACCCAGCAATGGTTTCCTATCATGGAGCA |
| | GATGCAGCCAGGAGATTCATTCTAACCATCTTAAATATGGTATTTAACCTTTTCCAAC |
| | ACAAGAGTCTGGGTGTGCAGGTCAATCTTCGTGTGATAAAGCTTATTCTGCTCCATGA |
| | AACTCCACCAGAACTATATATTGGGCATCATGGAGAAAAAATGCTAGAGAGTTTTTGT |
| | AAGTGGCAACATGAAGAATTTGGCAAAAAGAATGATATACATTTAGAGATGTCAACAA |
| | ACTGGGGGGAAGACATGACTTCAGTGGATGCAGCTATACTTATAACAAGGAAAGATTT |
| | CTGTGTGCACAAAGATGAACCATGTGATACTGTTGGTATAGCTTACTTGAGTGGAATG |
| | TGTAGTGAAAAGAGAAAATGTATTATTGCTGAAGACAATGGCTTGAATCTTGCTTTTA |
| | CAATTGCTCATGAATGGGTCACAACATGGGCATTAACCATGACAAATGACCACCCATC |
| | GTGTGCTGATGGTCTTCATATCATGTCTGGTGAATGGATTAAAGGACAGAATCTTGGT |
| | GACGTTTCATGGTCTCGATGTAGCAAAGGAAGATTTGGAAGATTTCTCAGGTCAAAGG |
| | CCAGTAACTGCTTGCTACAAACAAATCCGCAGAGTGTCAATTCTGTGATGGTTCCCTC |
| | CAAGCTGCCAGGGATGACATACACTGCTGATGAACAATGCCAGATCCTTTTTGGGCCA |
| | TTGGCTTCTTTTTGTCAGGAGATGCAGCATGTTATTTGCACAGGATTATGGTGCAAGG |
| | TAGAAGGTGAGAAAGAATGCAGAACCAAGCTAGACCCACCAATGGATGGAACTGACTG |
| | TGACCTTGGTAAGTGGTGTAAGGCTGGAGAATGTACCAGCAGGACCTCAGCACCTGAA |
| | CATCTGGCCGGAGAGTGGAGCCTGTGGAGTCCTTGTAGCCGAACCTGCAGTGCTGGGA |
| | TCAGCAGTCGAGAGCGCAAATGTCCTGGGCTAGATTCTGAAGCAAGGGATTGTAATGG |
| | TCCCAGAAAACAATACAGAATATGTGAGAATCCACCTTGTCCTGCAGGTTTGCCTGGA |
| | TTCAGAGACTGGCAATGTCAGGCTTATAGTGTTAGAACTTCCTCCCCAAAGCATATAC |
| | TTCAGTGGCAAGCTGTCCTGGATGAAGkAAAACCATGTGCCTTGTTTTGCTCTCCTGT |
| | TGGAAAAGAACAGCCTATTCTTCTATCAGAAAAAGTGATGGATGGAACTTCTTGTGGC |
| | TATCAGGGATTAGATATCTGTGCAAATGGCAGGTGCCAGAAAGTTGGCTGTGATGGTT |
| | TATTAGGGTCTCTTGCAAGAGAAGATCATTGTGGTGTATGCAATGGCAATGGAAAATC |
| | ATGCAAGATCATTAAAGGGGATTTTAATCACACCAGAGGAGCAGGTTATGTACAAGTG |
| | CTGGTGATACCTGCTGGAGCAAGAAGAATCAGTTGTGGAGGAAAAAAGCCGGCACATA |
| | GCTATTTAGCTCTCCGAGATGCTGGCAACAGTCTATTTAATAGTGACTGGAGAATTGA |
| | ACACTCTGGAGCCTTCAATTTGGCTGGAACTACCGTTCATTATGTAAGACGAGGCCTC |
| | TGGGAGAAGATCTCTGCCAAAGGTCCTACTACAGCACCTTTACATCTTCTGGTGCTCC |
| | TGTTTCAGGATCAGAATTATGGTCTTCACTATGAATACACTATCCCATCAGACCCTCT |
| | TCCAGAAAACCAGAGCTCTAAAGCACCTGAGCCCCTCTTCATGTGGACACACACAAGC |
| | TGGGAAGATTGCGATGCCACTTGTGGAGGAGGAGAAAGGAAGACAACAGTGTCCTGCA |
| | CAAAAATCATGAGCAAAAATATCAGCATTGTGGACAATGAGATGCAAAAATACTTAAC |
| | CAAGCCAGAGACCACAGATTCGAAAGTGCAATGAGCAACCATGTCACAAAGGGAATAT |
| | CTAATAAGTCGTGTGAGTGCTACAAGCCAGGCAATAGAGAGCAAAGAAAAGGCCAGTC |
| | CCCATTGGTTGAATGGAGAAGCCCTTCTAGGAGGAATGGGCGTGGGGCTGGCTGTCAA |
| | GGATCCAGGCACAGGATTCTACAAATATCATGAGGTGAAAATAGAAAGTGTTTGGTGG |
| | ATGATGACAGAATGGACCCCTTGTTCACGAACTTGTGGAAAAGGAATGCAGAGCAGAC |
| | AAGTGGCCTGTACCCAACAACTGAGCAATGGAACACTGATTAGAGCCCGAGAGAGGGA |
| | CTGCATTGGGCCCAAGCCCGCCTCTGCCCAGCGCTGTGAGGGCCAGGACTGCATGACC |
| | GTGTGGGAGGCGGGAGTGTGGTCTGAGTGTTCAGTCAAGTGTGGCAAAGGCATACGTC |
| | ATCGGACCGTTAGATGTACCAACCCAAGAAAGAAGTGTGTCCTCTCTACCAGACCCAG |
| | GGAGGCTGAAGACTGTGAGGATTATTCAAAATGCTATGTGTGGCGAATGGGTGACTGG |
| | TCTAAGTGCTCAATTACCTGTGGCAAAGGAATGCAGTCCCGTGTAATCCAATGCATGC |
| | ATAAGATCACAGGAAGACATGGAAATGAATGTTTTTCCTCAGAAAAACCTGCAGCATA |
| | CAGGCCATGCCATCTTCAACCCTGCAATGAGAAAATTAATGTAAATACCATAACATCA |
| | CCCAGACTGGCTGCTCTGACTTTCAAGTGCCTGGGAGATCAGTGGCCAGTGTACTGCC |
| | GAGTGATACGTGAAAAGAACCTATGTCAGGACATGCGGTGGTATCAGCGCTGCTGTGA |
| | AACATGCAGGGACTTCTATGCCCAAAAGCTGCAGCAGAAGAGTTGACCTCTAGCAGGC |
| | TGGCTGGATCACAGCTCTTGGCAATTACATTATTTATAAACACACACACTAGCATGTT |
| | TTTCAGACCAAATATTATCAGATTACATATAATTTAATCAAATTAATTTATTTTTTTG |
| | CCTGCCAAACATCCAATGTGGTCCTTGTTTTGG |
| | ORF Start: ATG at 16 ORF Stop: TGA at 3814 |
| | SEQ ID NO: 124 1266 aa MW at 140434.5kD |
| NOV37b, | MRLTHICCCCLLYQLGFLSNGIVSELQFAPDREEWEVVFPALWRREPVDPAGGSGGSA |
| CG133589-02 | |
| Protein | DPGWVRGVGGGGSARAQkAGSSREVRSVAPVPLEEPVEGRSESRLRPPPPSEGEEDEE |
| Sequence | |
| | LESQFLPRGSSGAkALSPGAPASWQPPPPPQPPPSRPPAQHAEPDGDEVLLRIPAFSR |
| | DLYLLLRRDGRFLAPRFAVEQRPNPGPGPTGAASAPQPPAPPDAGCFYTGAVLRHPGS |
| | LASFSTCGGGLMGPIQLNEDFIFTERLNDTMAITGHPHRVYRQKREMEEKVTEKSALH |
| | SHYCGTISDKCRPRSRKIAESGRGKRYSYKLPQEYNIETVVVADPAMVSYHGADAARR |
| | FILTILNMVFNLFQHKSLGVQVNLRVIKLILLHETPPELYIGHHCEKMLESPCKWQHE |
| | EFGKKNDIHLEMSTNWGEDMTSVUKAILITPKDFCVHKDEPCDTVGIAYLSGMCSEKR |
| | KCIIAEDNGLNLAFTIAHEMGHNMGINHDNDHPSCADGLHTMSGEWIKGQNLGDVSWS |
| | RCSKEDLERFLRSKASNCLLQTHPQSVNSVMVPSKLPGMTYTADEQCQILFGPLASFC |
| | QEMQHVICTGLWCKVEGEKECRTKLDPPMDGTDCDLGKWCKAGECTSRTSAPEHLAGE |
| | WSLWSPCSRTCSAGISSRERKCPGLDSEARDCNGPRKQYRICENPPCPAGLPGPRDWQ |
| | CQAYSVRTSSPKHILQWQAVLDEEKPCALFCSPVGKEQPILLSEKVMDGTSCGYQGLD |
| | ICANGRCQKVGCDGLLGSLARPDHCGVCNGNGKSCKIIKGDPNHTRGAGYVEVLVIPA |
| | GARRIKVVEEKPAHSYLALRDAGKQSINSDWKIEHSGAFNLAGTTVHYVRRGLWEKIS |
| | AKGPTTAPLHLLVLLFQDQNYGLHYEYTIPSDPLPENQSSKAPEPLFMWTHTSWEDCD |
| | ATCGGGERKTTVSCTKIMSKNISIVDNEKCKYLTKPEPQIRKCNEQPCQTREYLISRV |
| | SATSQAIESKEKASPHWLNGEALLGGMGVGLAVKDPGTGFYKYHEVKIESVWWMMTEW |
| | TPCSRTCGKGMQSRQVACTQQLSNGTLIRARERDCIGPKPASAQRCEGQDCMTVWEAG |
| | VWSECSVKCGKCIRHRTVRCTNPRKKCVLSTRPREAEDCEDYSKCYVWRMGDWSKCSI |
| | TCGKGMQSRVIQCMHKITGRHGNECFSSEKPAAYRPCHLQPCNEKINVNTITSPRLAA |
| | LTFKCLGDQWPVYCRVIREKNLCQDMRWYQRCCETCRDFYAQKLQQKS |

Sequence comparison of the above protein sequences yields the following sequence relationships shown in Table 37B.

TABLE 37B
Comparison of NOV37a against NOV37b

| | | Identities/ |
|---|---|---|
| Portein | | Similarities for |
| Sequence | Match Residues | the Matched Region |
| NOV37b | 1 . . . 663 | 574/687 (83%) |
| | 488 . . . 1157 | 585/687 (84%) |

Further analysis of the NOV37a protein yielded the following properties shown in Table 37C.

TABLE 37C
Protein Sequence Properties NOV37a

| | |
|---|---|
| PSort | 0.3000 probability located in microbody (peroxisome); 0.3000 |
| analysis: | probability located in nucleus; 0.1000 probability located in |
| | mitochondrial matrix space; 0.1000 probability located in |
| | lysosome (lumen) |
| SignalP | No Known Signal Sequence Predicted |
| analysis: | |

A search of the NOV37a protein against the Geneseq database a proprietary database that contains sequences published in patents and patent publication, yielded several homologous proteins shown in Table 37D.

TABLE 37D
Geneseq Results for NOV37a

| | | NOV37a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| ABG01904 | Novel human diagnostic protein #1895- | 1 . . . 633 | 632/633 (99%) | 0.0 |
| | Homo sapiens, 634 aa. | 1 . . . 633 | 632/633 (99%) | |
| | [WO200175067-A2, 11 Oct. 2001] | | | |
| ABG01904 | Novel human diagnostic protein #1895- | 1 . . . 633 | 632/633 (99%) | 0.0 |
| | Homo sapiens, 634 aa. | 1 . . . 633 | 632/633 (99%) | |
| | [WO200175067-A2, 11 Oct. 2001] | | | |
| AAE21003 | Human protease #5-Homo sapiens, | 1 . . . 647 | 450/679 (66%) | 0.0 |
| | 969 aa. [WO200229026-A2, | 250 . . . 900 | 490/679 (71%) | |
| | 11 Apr. 2002] | | | |
| AAE21002 | Human protease #4-Homo sapiens, | 1 . . . 647 | 450/679 (66%) | 0.0 |
| | 1213 aa. [WO200229026-A2, | 494 . . . 1144 | 490/679 (71%) | |
| | 11 Apr. 2002] | | | |
| AAU72900 | Human metalloprotease partial protein | 1 . . . 647 | 450/679 (66%) | 0.0 |
| | sequence #12-Homo sapiens, 1094 aa. | 375 . . . 1025 | 489/679 (71%) | |
| | [WO200183782-A2, 8 Nov. 2001] | | | |

In a BLAST search of public sequence datbases, the NOV37a protein was found to have homology to the proteins shown in the BLASTP data in Table 37E.

TABLE 37E
Public BLASTP Results for NOV37a

| | | NOV37a | | |
|---|---|---|---|---|
| Protein | | Match | Identities/ | Expect |
| Number | Protein/Organism/Length | Residues | Matched Portion | Value |
| Q8TE59 | ADAMTS-19-Homo sapiens | 1 . . . 647 | 449/679 (66%) | 0.0 |
| | (Human), 1207 aa. | 488 . . . 1138 | 489/679 (71%) | |
| QSTE56 | Metalloprotease disintegrin 17, with | 224 . . . 647 | 184/434 (42%) | e−101 |
| | thrombospondin domains-Homo | 628 . . . 1023 | 256/434 (58%) | |
| | sapiens (Human), 1095 aa. | | | |
| CAC38921 | Sequence 2 from Patent WO0131034- | 2 . . . 608 | 207/637 (32%) | 7e−75 |
| | Homo sapiens (Human), 1686 aa. | 395 . . . 999 | 295/637 (45%) | |
| Q9EPX2 | Papilin-Mus musculus (Mouse), | 220 . . . 647 | 149/455 (32%) | 2e−56 |
| | 1280 aa. | 108 . . . 534 | 218/455 (47%) | |
| Q9U8G8 | Lacunin precursor-Manduca sexta | 222 . . . 663 | 153/464 (32%) | 7e−54 |
| | (Tobacco hawkmoth) (Tobacco | 143 . . . 593 | 212/464 (44%) | |
| | hornworm), 3198 aa. | | | |

PFam analysis predicts that the NOV37a protein contains the domains Shown in the Table 37F.

TABLE 37F
Domain Analysis of NOV37a

| | | Identities/ | |
|---|---|---|---|
| Pfam | NOV37a | Similarities for | Expect |
| Domain | Match Region | the Matched Region | Value |
| tsp_1 | 426 . . . 482 | 13/62 (21%) | 0.091 |
| | | 40/62 (65%) | |
| tsp_1 | 542 . . . 601 | 18/67 (27%) | 0.011 |
| | | 40/67 (60%) | |
| tsp_1 | 603 . . . 653 | 22/57 (39%) | 0.00014 |
| | | 36/57 (63%) | |

### Example 38

The NOV38 clone was analyzed, and the nucleotide and encoded polypeptide sequences are shown in Table 38A.

TABLE 38A
NOV38 Sequence Analysis

| | |
|---|---|
| | SEQ ID NO: 125 735 bp |
| NOV38a, | AGTGGCAAGATGGCGTCCCTGGATCGGGTGAAGGTACTGGTGTTGGGAGACTCAGGTG |
| CG133668-01 | |
| DNA | TTGGGAAATCTTCGTTAGTCCATCTCCTATGCCAAAATCAAGTGCTGGGAAATCCATC |
| Sequence | |
| | ATGGACTGTGGGCTGCTCAGTGGATGTCAGAGTTCATGATTACAAAGAAGGAACCCCA |
| | GAAGAGAAGACCTACTACATAGAATTATGGGATGTTGGAGGCTCTGTGGGCAGTGCCA |
| | GCAGCGTGAAAAGCACAAGAOCAGTATTCTACAACTCCGTAAATGGTATTATTTTCGT |
| | ACACGACTTAACAAATAAGAAGTCCTCCCAAAACTTGCGTCGTTGGTCATTGGAAGCT |
| | CTCAACAGGGATTTGGTGCCAACTGGAGTCTTGGTGACAAATGGGGATTATGATCAAG |
| | AACAGTTTGCTGATAACCAAATACCACTGTTGGTAATAGGGACTAAACTGGACCAGAT |
| | TCATGAAACAAAGCGCCATGAAGTTTTAACTAGGACTGCTTTCCTGGCTGAGGATTTC |
| | AATCCAGAAGAAATTAATTTGGACTGCACAAATCCACGGTACTTAGCTGCAGGTTCTT |
| | CCAATGCTGTCAAGCTCAGTAGGTTTTTTGATAAGGTCATAGAGAAGAGATACTTTTT |
| | AAGAGAAGGTAATCAGATTCCAGGCTTTCCTGATCGGAAAAGATTTGGGGCAGGAACA |
| | TTAAAGAGCCTTCATTATGACTGAATTACACTCATCCTT |
| | ORF Start: ATG at 10 ORF Stop: TGA at 718 |
| | SEQ ID NO: 126 236 aa MW at 26422.6kD |
| NOV38a, | MASLDRVKVLVLGDSGVGKSSLVHLLCQNQVLGNPSWTVGCSVDVRVHDYKEGTPEEK |
| CG133668-01 | |
| Protein | TYYIELWDVGGSVGSASSVKSTRAVFYNSVNGIIFVHDLTNKKSSQNLRRWSLEALNR |
| Sequence | |
| | DLVPTGVLVTNGDYDQEQFADNQIPLLVIGTKLDQIHETKRHEVLTRTAFLAEDFNPE |
| | EINLDCTNPRYLAAGSSNAVKLSRFFDKVIEKRYFLREGNQIPGFPDRKRFGAGTLKS |
| | LHYD |
| | SEQ ID NO: 127 739 bp |
| NOV38b, | AGTGGCAAGATCGCGTCCCTGGATCGGGTGAAGGTACTGGTGTTGGGAGACTCAGGTG |
| CG133668-02 | |
| DNA | TTGGGAAATCTTCGTTAGTCCATCTCCTATGCCAkAATCAAGTGCTGGGAAATCCATC |
| Sequence | |
| | ATGGACTGTGGGCTGCTCAGTGGATGTCAGAGTTCATGATTACAAAGAAGGAACCCCA |
| | GAAGAGAAGACCTACTACATAGAATTATGGGATGTTGGAGGCTCTGTGGGCAGTGCCA |
| | GCAGCGTGAAAAGCACAAGAGCAGTATTCTACAACTCCGTAAATGGTATTATTTTCGT |
| | ACACGACTTAACAAATAAGAAGTCCTCCCAAAACTTGCGTCGTTGGTCATTGGAAGCT |
| | CTCAACAGGGATTTGGTGCCAACTGGAGTCTTGGTGACAAATGGGGATTATGATCAAG |
| | AACAGTTTGCTGATAACCAAATACCACTGTTGGTAATAGGGACTAAACTGGACCAGAT |
| | TCATGAAACAAAGCGCCATGAAGTTTTAACTAGGACTGCTTTCCTGGCTGAGGATTTC |
| | AATCCAGAAGAAATTAATTTGGACTGCACAAATCCACGGTACTTAGCTGCAGGTTCCT |
| | CCAATGCTGTCAAGCTCAGTAGGTTTTTTGATAAGGTCATAGAGAAGAGATACTTTTT |
| | AAGAGAAGGTAATCAGATTCCAGGCTTTCCTGATCGGAAAAGATTTGGGGCAGGAACA |
| | TTAAAGAGCCTTCATTATGACTGAATTACACTCATCCTAAGGG |
| | ORF Start: at 10 ORF Stop: TGA at 718 |
| | SEQ ID NO: 128 236 aa MW at 26404.5kD |
| NOV38b, | IASLDRVKVLVLGDSGVGKSSLVHLLCQNQVLGNPSWTVGCSVDVRVHDYKEGTREEK |
| CG133668-02 | |
| Protein | TYYIELWDVGGSVGSASSVKSTRAVFYNSVNGIIFVHDLTNKKSSQNLRRWSLEALNR |
| Sequence | |
| | DLVPTGVLVTNGDYDQEQFADNQIPLLVIGTKLDQTHETKRHEVLTRTAFLAEDFNPE |
| | EINLDCTNPRYLAAGSSNAVKLSRFFDKVIEKRYFLREGNQTPGFPDRKRFGAGTLKS |
| | LHYD |

Sequence comparison of the above protein sequences yields tile following, sequence relationships shown in Table 38B.

TABLE 38B
Comparison of NOV38a against NOV38b

| | | Identities/ |
|---|---|---|
| Protein | NOV38a Residues/ | Similarities for |
| Sequence | Match Residues | the Matched Region |
| NOV38b | 1 . . . 236 | 222/236 (94%) |
| | 1 . . . 236 | 223/236 (94%) |

Further analysis of the NOV38a protein yielded the following properties shown in Table 38C.

TABLE 38C
Protein Sequence Properties NOV38a

| | |
|---|---|
| analysis: | (peroxisome); 0.1000 probability located in mitochondrial |
| | matrix space; 0.1000 probability located in lysosome (lumen) |
| SignalP | No Known Signal Sequence Predicted |
| analysis: | |

A search of the NOV38a protein against the Geneseq database, a proprietary database that contains sequences published in patents and patent publication yielded several homologous proteins shown in Table 38D.

TABLE 38D
Geneseq Results for NOV38a

| | | NOV38a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAE21568 | Human G-protein (47324) polypeptide- | 1 . . . 236 | 236/236 (100%) | e−136 |
| | Homo sapiens, 236 aa. [WO200218425- | 1 . . . 236 | 236/236 (100%) | |
| | A2, 7 Mar. 2002] | | | |
| AAU17369 | Novel signal transduction pathway | 1 . . . 231 | 210/231 (90%) | e−114 |
| | protein, Seq ID 934-Homo sapiens, | 4 . . . 232 | 212/231 (90%) | |
| | 269 aa [WO200154733-A1, | | | |
| | 2 Aug. 2001] | | | |
| AAY12450 | Human 5′ EST secreted protein SEQ ID | 1 . . . 125 | 121/125 (96%) | 3e−64 |
| | NO: 481-Homo sapiens 125 aa. | 1 . . . 125 | 121/125 (96%) | |
| | [WO9906548-A2, 11 Feb. 1999] | | | |
| ABB60970 | Drosophila melanogaster polypeptide | 1 . . . 231 | 113/264 (42%) | 2e−47 |
| | SEQ ID NO 9702-Drosophila | 6 . . . 264 | 157/264 (58%) | |
| | melanogaster, 279 aa. [WO200171042- | | | |
| | A2, 27 Sep. 2001] | | | |
| AAG49196 | Arabidopsis thaliana protein fragment | 6 . . . 150 | 54/155 (34%) | 4e−19 |
| | SEQ ID NO. 62211-Arabidopsis | 228 . . . 369 | 87/155 (55%) | |
| | thaliana, 606 aa. [EP1033405-A2, | | | |
| | 6 Sep. 2000] | | | |

In a BLAST search of public sequence datbases, the NOV38a protein was found to have homology to the proteins shown in the BLASTP data in Table 38E.

TABLE 38E
Public BLASTP Results for NOV38a

| | | NOV38a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q8WUD3 | Similar to RIKEN cDNA | 1 . . . 236 | 234/236 (99%) | e−134 |
| | 4930553C05 gene−Homo sapiens | 1 . . . 236 | 234/236 (99%) | |
| | (Human), 236 aa. | | | |
| Q9D4V7 | 4930553C05Rik protein-Mus | 1 . . . 236 | 218/236 (92%) | e−124 |
| | musculus (Mouse), 236 aa. | 1 . . . 236 | 221/236 (93%) | |
| Q9D0M6 | 4930553C05Rik protein-Mus | 1 . . . 129 | 123/129 (95%) | 1e−66 |
| | musculus (Mouse), 129 aa. | 1 . . . 129 | 124/129 (95%) | |
| Q8SZD5 | RE04047p-Drosophila | 1 . . . 231 | 113/264 (42%) | 4e−47 |
| | melanogaster (Fruit fly), 274 aa. | 1 . . . 259 | 157/264 (58%) | |
| Q9VXA9 | CG4789 protein-Drosophila | 1 . . . 231 | 113/264 (42%) | 4e−47 |
| | melanogaster (Fruit fly), 279 aa. | 6 . . . 264 | 157/264 (58%) | |

PFam analysis predicts that the NOV38a protein contains the domains shown in the Table 38F.

TABLE 38F
Domain Analysis of NOV38a

| | | Identities/ | |
|---|---|---|---|
| Pfam | | Similarities for | Expect |
| Domain | NOV38a Match Region | the Matched Region | Value |
| Ras | 8 . . . 231 | 42/239 (18%) | 1e−06 |
| | | 144/239 (60%) | |

### Example 39

The NOV39 clone was analyzed, and the nucleotide and encoded polypeptide sequences are shown in Table 39A.

TABLE 39A
NOV39 Sequence Analysis

| | |
|---|---|
| | SEQ ID NO: 129 3771 bp |
| NOV39a, | GGAGCCCTCCAGAGCGCTTCTCGGCTGCCTAGCGAGCGCCGCCGCTGCCGCCCCGCCG |
| CG133750-01 | |
| DNA | GGGGAGGATGGAGCAGGGGCCGGGGCCGAGGAGGAGGAGGAGGAGGAGGAGGAGGCGG |
| Sequence | |
| | CGGCGGCGGTGGGCCCCGGGGCAGCTGGGCTGCGACGCGCCGCTGCCCTACTGGACGG |
| | CCGTGTTCGAGTACGAGGCGGCGGGCGAGGACGAGCTGACCCTGCGGCTGGGCGACGT |
| | GGTGGAGGTCCTCTCCAAGGACTCGCAGGTGTCCGGCGACGAGGGCTGGTGGACCGGG |
| | CAGCTGAACCAGCGGGTGGGCATCTTCCCCAGCAACTACGTGACCCCGCGCAGCGCCT |
| | TCTCCAGCCGCTGCCAGCCCGGCGGCGAGGACCCCAGTTGCTACCCGCCCATTCAGTT |
| | GTTAGAPATTGATTTTGCGGAGCTCACCTTGGAAGAGATTATTGGCATCGGGGGCTTT |
| | GGGAAGGTCTATCGTGCTTTCTGGATAGGGGATGAGGTTCCTGTGAAAGCAGCTCGCC |
| | ACGACCCTGATGAGGACATCAGCCAGACCATAGAGAATGTTCCCCAAGAGGCCAAGCT |
| | CTTCGCCATGCTGAAGCACCCCAACATCATTGCCCTAAGAGGGGTATGTCTGAAGGAG |
| | CCCAACCTCTGCTTGGTCATGGAGTTTGCTCGTGGAGGACCTTTGAATAGAGTGTTAT |
| | CTGGGAAAAGGATTCCCCCAGACATCCTGGTGAATTGGGCTGTGCAGATTGCCAGACG |
| | GATGAACTACTTACATGATGAGGCAATTGTTCCCATCATCCACCGCGACCTTAAGTCC |
| | AGCAACATATTGATCCTCCAGAAGGTGGAGAATGGAGACCTGAGCAACAAGATTCTGA |
| | AGATCACTGATTTTGGCCTGGCTCGGGAATGGCACCGAACCACCAAGATGAGTGCGGC |
| | AGGGACGTATGCTTGGATGGCACCCGAAGTCATCCGGGCCTCCATGTTTTCCAAAGGC |
| | AGTGATGTGTGGAGCTATGGGGTGCTACTTTGGGAGTTGCTGACTGGTGAGGTGCCCT |
| | TTCGAGGCATTGATGGCTTAGCAGTCGCTTATGGAGTGGCCATGAACAAACTCGCCCT |
| | TCCTATTCCTTCTACGTGCCCAGAACCTTTTGCCAAACTCATGGAAGACTGCTGGAAT |
| | CCTGATCCCCACTCACGACCATCTTTCACGAATATCCTGGACCAGCTAACCACCATAG |
| | AGGAGTCTGGTTTCTTTGAAATCCCCAAGGACTCCTTCCACTGCCTGCAGGACAACTG |
| | GAAACACGAGATTCAGGAGATGTTTCACCAACTCAGGGCCAAAGAAAAGGAACTTCGC |
| | ACCTGGGAGGAGGAGCTGACGCGGGCTGCACTGCAGCAGAAGAACCAGGAGGAACTGC |
| | TGCGGCGTCGGGAGCAGGAGCTGGCCGAGCGGGAGATTGACATCCTGGAACGGGAGCT |
| | CAACATCATCATCCACCAGCTGTGCCAGGAGAAGCCCCGGGTGAAGAAACGCAAGGGC |
| | AAGTTCAGGAAGAGCCGGCTGAAGCTCAAGGATGGCAACCGCATCAGCCTCCCTTCTG |
| | ATTTCCAGCACAAGTTCACGGTGCAGGCCTCCCCTACCATGGATAAAAGGAAGAGTCT |
| | TATCAACAGCCGCTCCAGTCCTCCTGCAAGCCCCACCATCATTCCTCGCCTTCGAGCC |
| | ATCCAGTTGACACCACGTGAAAGCAGCAAAACCTGGGGCAGGAGCTCAGTCGTCCCAA |
| | GCCAGGGACGCTTGGTCAGAAAGAGCTTGCCTCGGGAGATGAAGGATCCCCTCAGAGA |
| | CGTGAGAAAGCTAATGGTTTAAGTACCCCATCAGAATCTCCACATTTCCACTTGGGCC |
| | TCAAGTCCCTGGTAGATGGATATAAGCAGTGGTCGTCCAGTGCCCCCAACCTGGTGAA |
| | GGGCCCAAGGAGTAGCCCGGCCCTGCCAGGGTTCACCAGCCTTATGGAGATGGCCTTG |
| | CTGGCAGCCAGTTGGGTGGTGCCCATCGACATTGAAGAGCATGAGCACAGTGAAGGCC |
| | CAGGCACTGGAGAGAGTCGCCTACAGCATTCACCCAGCCAGTCCTACCTCTGTATCCC |
| | ATTCCCTCGTGGAGAGGATGGCGATGGCCCCTCCAGTGATCGAATCCATGAGGAGCCC |
| | ACCCCAGTCATCTCGGCCACGAGTACCCCTCAGCTGACGCCAACCAACAGCCTCAAGC |
| | GGGGCGGTGCCCACCACCGCCGCTGCGAGGTGGCTCTGCTCGGCTGTGGGCCTGTTCT |
| | GGCAGCCACAGGCCTACGGTTTGACTTGCTGGAAGCTGGCAAGTGCCAGCTGCTTCCC |
| | CTGGAGGAGCCTGAGCCACCAGCCCGGGAGGAGAAGAAAAGACGGGAGGGTCTTTTTC |
| | ACAGGTCCAGCCGTCCTCGTCGGAGCACCAGCCCCCCATCCCGAAAGCTTTTCATGAA |
| | GGAGGAGCCCATGCTGTTGCTAGGAGACCCCTCTGCCTCCCTGACGCTGCTCTCCCTC |
| | TCCTCCATCTCCGAGTGCAACTCCACACGCTCCCTGCTGCGCTCCGACAGCGATGAAA |
| | TTGTCGTGTATGAGATGCCAGTCAGCCCAGTCGAGGCCCCTCCCCTGAGTCCATGTAC |
| | CCACAACCCCCTGGTCAATGTCCGAGTAGAGCGCTTCAAACGACATCCTAACCAATCT |
| | CTGACTCCCACCCATGTCACCCTCACCACCCCCTCGCAGCCCAGCAGTCACCGGCGGA |
| | CTCCTTCTGATGGGGCCCTTAAGCCAGAGACTCTCCTACCCAGCACGAGCCCCTCCAG |
| | CAATCGGTTGAGCCCCAGTCCTGGACCAGGAATGTTGAAAACCCCCAGTCCCAGCCGA |
| | GACCCAGGTGAATTCCCCCGTCTCCCTGACCCCAATGTGGTCTTCCCCCCAACCCCAA |
| | GGCGCTGGAACACTCAGCACGACTCTACCTTGGAGAGACCCAAGACTCTGGAGTTTCT |
| | GCCTCGGCCGCGTCCTTCTGCCAACCGGCAACGGCTGGACCCTTGGTGGTTTGTGTCC |
| | CCCAGCCATGCCCGCAGCACCTCCCCACCCAACAGCTCCAGCACAGAGACGCCCAGCA |
| | ACCTGGACTCCTCCTTTGCTAGCAGTAGCAGCACTGTAGAGGAGCGGCCTGGACTTCC |
| | AGCCCTGCTCCCGTTCCAGGCAGGGCCGCTGCCCCCGACTGAGCGGACGCTCCTGGAC |
| | CTGGATGCAGAGGGGCAGAGTCAGGACAGCACCGTGCCGCTGTGCAGAGCGGAACTGA |
| | ACACACACAGGCCTGCCCCTTATGAGATCCAGCAGGAGTTCTGGTCTTAGCACGAAAA |
| | GGATTGGGGCGGGCAAGGGCGACAGCCAGCGGAGATGAGGGGAGCTGGCGGGCACAGC |
| | CCTTTCTCAGGGTTCGACCCCCTGAGATCCAGCCCTACTTCTTGCACTGATAATGCAC |
| | TTTGAAGATGGAAGGGATGGAAACAGGGCCACTTCAGAGGGTCTCCTGCCCTGCAGGG |
| | CCTTTCTACCCGTGTCCACTGGAGGGGCTGTGGCCATCAGCTCTGGCTGTGTAGCGGA |
| | GGAAGGGGTGCATGCATGTCCCCCACCCTCCACAGTCTTCCTTGCCTTTAGAGTGACC |
| | CTGCACAGTCACTCAGCCAAATCTGTCTGCTGCTCCCTCTCCTCAGCCAGTTGGGTGT |
| | GCCCA |
| | ORF Start: ATG at 66 ORF Stop: TAG at 3354 |
| | SEQ ID NO: 130 1096 aa MW at 122187.8kD |
| NOV39a, | MEQGPGPRRRRRRRRRRRRRWAPGQLGCDAPLPYWTAVFEYEAAGEDELTLRLGDVVE |
| CG133750-01 | |
| Protein | VLSKDSQVSGDEGWWTGQLNQRVGTPPSNYVTPRSAFSSRCQPGGEDPSCYPPIQLLE |
| Sequence | |
| | TDFAELTLEEIIGIGGFGKVYRAFWIGDEVAVKAARHDPDEDISQTIENVRQEAKLFA |
| | MLKHPNTIALRGVCLKEPNLCLVMEFARGGPLNRVLSGKRIPPDILVNWAVQIARGMN |
| | YLHDEAIVPIIHRDLKSSNTLILQKVENGDLSNKILKITDFGLAREWHRTTKMSAAGT |
| | YAWMAPEVIRASMFSKGSDVWSYGVLLWELLTGEVPFRGIDGLAVAYGVAMNKLALPI |
| | PSTCPEPFAKLMEDCWNPDPHSRPSFTNILDQLTTIEESGFFEMPKDSFHCLQDNWKH |
| | EIQEMFDQLRAKFKELRTWEEELTRAALQQKNQEELLRRREQELAEREIDILERELNI |
| | IIHQLCQEKPRVKKRKGKFRKSRLKLKDGNRISLPSDFQHKFTVQASPTMDKRKSLIN |
| | SRSSPPASPTIIPRLRAIQLTPGESSKThGRSSVVPKEEGEEEEKRAPKKKGRTWGPG |
| | TLGQKELASGDEGSPQRREKANGLSTPSESPHPHLGLKSLVDGYKQWSSSAPNLVKGP |
| | RSSPALPGFTSLMEMALLAASWVVPIDIEEDEDSEGPGSGESRLQHSPSQSYLCIPFP |
| | RGEDGDGPSSDGIHEEPTPVNSATSTPQLTPTNSLKRGGAHHRRCEVALLGCGAVLAA |
| | TGLGPDLLEAGKCQLLPLEEPEPPAREEKKRREGLPQRSSRPRRSTSPPSRKLFKKEE |
| | PMLLLGDPSASLTLLSLSSISECNSTRSLLRSDSDEIVVYEMPVSPVEAPPLSPCTHN |
| | PLVNVRVERFKRDPNQSLTPTHVTLTTPSQPSSHRRTPSDGALKPETLLASRSPSSNG |
| | LSPSPGAGMLKTPSPSRDPGEFPRLPDPNVVPPPTPRRWNTQQDSTLERPKTLEFLPR |
| | PRPSANRQRLDPWWFVSPSHARSTSPANSSSTETPSNLDSCFASSSSTVEERPGLPAL |
| | LPFQAGPLPPTERTLLDLDAEGQSQDSTVPLCRAELNTHRPAPYEIQQEFWS |

Further analysis of the NOV39a protein yielded the following properties shown in Table 39B.

TABLE 39B
Protein Sequence Properties NOV39a

| | |
|---|---|
| PSort | 0.7999 probability located in mitochondrial inner |
| analysis: | membrane; 0.6064 probability located in nucleus; 0.6000 |
| | probability located in mitochondrial matrix space; 0.6000 |
| | probability located in mitochondrial intermembrane space |
| SignalP | No Known Signal Sequence Predicted |
| analysis: | |

A search of the NOV39a protein against the Geneseq database, a proprietary database that contains sequences published in patents and patent publication, yielded several homologous proteins shown in Table 39C.

TABLE 39C
Geneseq Results for NOV39a

| | | NOV39a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAE21717 | Human PKIN-12 protein-Homo | 4 . . . 1096 | 1037/1109 (93%) | 0.0 |
| | sapiens, 1097 aa. [WO200218557-A2, | 26 . . . 1097 | 1038/1109 (93%) | |
| | 7 Mar. 2002] | | | |
| AAE11775 | Human kinase (PKIN)-9 protein- | 4 . . . 1096 | 991/1093 (90%) | 0.0 |
| | Homo sapiens, 1046 aa. | 26 . . . 1046 | 993/1093 (90%) | |
| | [WO200181555-A2, 1 Nov. 2001] | | | |
| AAB85513 | Human protein kinase SGK067-Homo | 35 . . . 733 | 420/722 (58%) | 0.0 |
| | sapiens, 719 aa. [WO200155356-A2, | 43 . . . 712 | 520/722 (71%) | |
| | 2 Aug. 2001] | | | |
| ABB58999 | Drosophila melanogaster polypeptide | 35 . . . 560 | 274/526 (52%) | e−147 |
| | SEQ ID NO 3789 -Drosophila | 48 . . . 541 | 350/526 (66%) | |
| | melanogaster, 1020 aa. | | | |
| | [WO200171042-A2, 27 Sep. 2001] | | | |
| AAU78826 | Multiple lineage kinase 1 (MLK1)- | 62 . . . 251 | 189/190 (99%) | e−109 |
| | Unidentified, 194 aa. [WO200214536- | 5 . . . 194 | 190/190 (99%) | |
| | A2, 21 Feb. 2002] | | | |

In a BLAST search of public sequence databases, the NOV39a protein was found to have homology to the proteins shown in the BLASTP data in Table 39D.

TABLE 39D
Public BLASTP Results for NOV39a

| | | NOV39a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9H2N5 | Mixed lineage kinase MLK1-Homo | 31 . . . 1096 | 1066/1066 (100%) | 0.0 |
| | sapiens (Human), 1066 aa | 1 . . . 1066 | 1066/1066 (100%) | |
| | (fragment). | | | |
| AAH30944 | Similar to mitogen-activated protein | 331 . . . 1096 | 694/769 (90%) | 0.0 |
| | kinase kinase kinase 9-Mus musculus | 1 . . . 732 | 709/769 (91%) | |
| | (Mouse), 732 aa (fragment). | | | |
| Q02779 | Mitogen-activated protein kinase | 33 . . . 1078 | 574/1066 (53%) | 0.0 |
| | kinase kinase 10 (EC 2.7.1.37) | 19 . . . 950 | 689/1066 (63%) | |
| | (Mixed lineage kinase 2) (Protein | | | |
| | kinase MST)-Homo sapiens | | | |
| | (Human), 954 aa. | | | |
| Q8WWN1 | Mixed lineage kinase 4beta-Homo | 35 . . . 1096 | 540/1112 (48%) | 0.0 |
| | sapiens (Human), 1036 aa. | 43 . . . 1036 | 688/1112 (61%) | |
| Q8VDG6 | Similar to mitogen-activated protein | 35 . . . 1094 | 491/1085 (45%) | 0.0 |
| | kinase kinase kinase 9-Mus musculus | 29 . . . 999 | 635/1085 (58%) | |
| | (Mouse), 1001 aa. | | | |

PFam analysis predicts that the NOV39a protein contains the domains shown in the Table 39E.

TABLE 39E
Domain Analysis of NOV39a

| | | Identities/ | |
|---|---|---|---|
| Pfam | | Similarities for | Expect |
| Domain | NOV39a Match Region | the Matched Region | Value |
| SH3 | 33 . . . 92 | 25/63 (40%) | 7.8e−15 |
| | | 50/63 (79%) | |
| Pkinase | 122 . . . 381 | 100/300 (33%) | 3.2e−94 |
| | | 217/300 (72%) | |

### Example 40

The NOV40 clone was analyzed, and the nucleotide and encoded polypeptide sequences are shown in Table 40A.

TABLE 40A
NOV40 Sequence Analysis

| | |
|---|---|
| | SEQ ID NO: 131 4803 bp |
| NOV40a, | AGAAGGAAGTGGCCTGGTGGATACACACCTGTTCTCTGCAGGCTCTTTCCTTGTCATG |
| CG133819-01 | |
| DNA | TTTCTCCCCTGGGGTTTGCAGCCTGGCTTTTCATTTTTAGTATCCTTCTGAAAGAAGA |
| Sequence | |
| | GAGAAAAATTTTCAGCAAAGAAGGCAAGTAAAAGATGAAAATTAAATTATGAGAATTA |
| | AAAAGACAACATTGAGCAGAGACATGAAAAAGGAAGGGAGGAAAAGGTGGAAAAGAAA |
| | AGAAGACAAGAAGCGAGTAGTGGTCTCTAACTTGCTCTTTGAAGGATGGTCTCACAAA |
| | GAGAACCCCAACAGACATCATCGTGGGAATCAAATCAAGACCAGCAAGTACACCGTGT |
| | TGTCCTTCGTCCCCAAAAACATTTTTGAGCAGCTACACCGGTTGGCCAATCTCTATTT |
| | TGTGGGCATTGCGGTTCTGAATTTTATCCCTGTGGTCAATGCTTTCCAGCCTGAGGTG |
| | AGCATGATACCAATCTGTGTTATCCTGGCAGTCACTGCCATCAAGGACGCTTGGGAAG |
| | ACCTCCGGAGGTACAAATCGGATAAAGTCATCAATAACCGAGAGTGCCTCATCTACAG |
| | CAGAAAAGAGCAGACCTATGTGCAGAAGTGCTGGAAGGATGTGCGCGTGCGAGACTTC |
| | ATCCAAATGAAATGCAATGAGATTGTCCCAGCAGACATACTCCTCCTTTTTTCCTCTG |
| | ACCCCAATGGGATATGCCATCTGGAAACTGCCAGCTTGGATGGAGAGACAAACCTCAA |
| | GCAAAGACGTGTCGTGAAGGGCTTCTCACAGCAGGAGGTACAGTTCGAACCAGAGCTT |
| | TTCCACAATACCATCGTGTGTGAGAAACCCAACAACCACCTCAACAAATTTAAGGGTT |
| | ATATGGAGCATCCTGACCAGACCAGGACTGGCTTTGGCTGTGAGAGTCTTCTGCTTCG |
| | AGGCTGCACCATCAGAAACACCGAGATGGCTGTTGGCATTGTCATCTATGCAGGCCAT |
| | GAGACGAAAGCCATGCTGAACAACAGTGGCCCCCGGTACAAACGCAGCAAGATTGAGC |
| | GGCGCATGAATATAGACATCTTCTTCTGCATTGGGATCCTCATCCTCATGTGCCTTAT |
| | TGGAGCTGTAGGTCACAGCATCTGGAATGGGACCTTTGAAAGACACCCTCCCTTCGAT |
| | GTGCCAGATGCCAATGGCAGCTTCCTTCCCAGTGCCCTTGGGGGCTTCTACATGTTCC |
| | TCACAATGATCATCCTGCTCCAGGTGCTGATCCCCATCTCTTTGTATGTCTCCATTGA |
| | GCTGGTGAAGCTCGGGCAAGTGTTCTTCTTGAGCAATGACCTTGACCTGTATGATGAA |
| | GAGACCGATTTATCCATTCAATGTCGAGCCCTCAACATCGCAGAGGACTTGGGCCAGA |
| | TCCAGTACATCTTCTCCGATAAGACGGGGACCCTGACAGAGAACAAGATGGTGTTCCG |
| | ACGTTGCACCATCATGGGCAGCGAGTATTCTCACCAAGAAAATGCTAAGCGACTGGAG |
| | ACCCCAAGGAGCTGGACTCAGATGGTGAAAGAGTGGACCCAATACCAATGCCTGTCCT |
| | TCTCGGCTAGATGGGCCCAGGATCCAGCAACTATGAGAAGCCAAAAAGGTGCTCAGCC |
| | TCTGAGGAGGAGCCAGAGTGCCCGGGTGCCCATCCAGGGCCACTACCGGCAAAGGTCT |
| | ATGGGGCACCGTGAAAGCTCACAGCCTCCTGTGGCCTTCAGCAGCTCCATAGAAAAAG |
| | ATGTAACTCCAGATAAAAACCTACTGACCAAGGTTCGAGATGCTGCCCTGTGGTTGGA |
| | GACCTTGTCAGACAGCAGACCTGCCAAGGCTTCCCTCTCCACCACCTCCTCCATTGCT |
| | GATTTCTTCCTTGACTTAACCATCTGCAACTCTGTCATGGTGTCCACAACCACCGAGC |
| | CCAGGCAGAGGGTCACCATCAAACCCTCAAGCAAGGCTCTGGGGACGTCCCTGGAGAA |
| | GATTCAGCAGCTCTTCCAGAAGTTGAAGCTATTGAGCCTCAGCCAGTCATTCTCATCC |
| | ACTGCACCCTCTGACACAGACCTCGGGGAGAGCTTAGGGGCCAACGTGGCCACCACAG |
| | ACTCGGATGAGAGAGATGATGCATCTGTGTGCAGTGGAGGTGACTCCACTGATGACGG |
| | TGGCTACAGGAGCAGCATGTGGGACCAGGGCGACATCCTGGAGTCTGGGTCAGGCACT |
| | TCCTTGGAGGAGGCATTGGAGGCCCCAGCCACAGACCTGGCCAGGCCTGAGTTCTGTT |
| | ACGAGGCTGAGAGCCCTGATGAGGCCGCCCTGGTGCACGCTGCCCATGCCTACAGCTT |
| | CACACTAGTGTCCCGGACACCTGAGCAGGTGACTGTGCGCCTGCCCCAGGGCACCTGC |
| | CTCACCTTCAGCCTCCTCTGCACCCTGGGCTTTGACTCTGTCAGGAAGAGAATGTCTG |
| | TGGTTGTGAGGCACCCACTGACTGGCGAGATTGTTGTCTACACCAAGGGTGCTGACTC |
| | GGTCATCATGGACCTGCTGGAAGACCCAGCCTGCGTACCTGACATTAATATGGAAAAG |
| | AAGCTGAGAPAAATCCGAGCCCGGACCCAAAAGCATCTAGACTTGTATGCAAGAGATG |
| | GCCTGCGCACACTATGCATTGCCAAGAAGGTTGTAAGCGAAGAGGACTTCCGGAGATG |
| | GGCCAGTTTCCGGCGTGAGGCTGAGGCATCCCTCGACAACCGAGATGAGCTTCTCATG |
| | GAAACTGCACAGCATCTGGAGAATCAACTCACCTTACTTGGAGCCACTGGGATCGAAG |
| | ACCGGCTGCAGGAAGGAGTTCCAGATACGATTGCCACTCTGCGGGAGGCTGGGATCCA |
| | GCTCTGGGTCCTGACTGGAGATAAGCAGGAGACAGCGGTCAACATTGCCCATTCCTGC |
| | AGACTGTTAAATCAGACCGACACTGTTTATACCATCAATACAGAGAATCAGGAGACCT |
| | GTGAATCCATCCTCAATTGTGCATTGGAAGAGCTAAAGCAATTTCGTGAACTACAGAA |
| | GCCAGACCGCAAGCTCTTTGGATTCCGCTTACCTTCCAAGACACCATCCATCACCTCA |
| | GAGCTGTGGTTCCAGAAGCTGGATTGGTCATCGATGGGTAAGACATTGAATGCCATCT |
| | TCCAGGGAAAGCTAGAGAAGAAGTTTCTGGAATTGACCCAGTATTGTCGGTCCGTCCT |
| | GTGCTGCCGCTCCACGCCACTCCAGAAGAGTATGATAGTCAAGCTGGTGCGAGACAAG |
| | TTGCGCGTCATGACCCTTTCCATAGGTGATGGAGCAAATGATGTAAGCATGATTCAAG |
| | CTGCTGATATTGGAATTGGAATATCTGGACAGGAAGGCATGCAGGCTGTCATGTCCAG |
| | CGACTTTGCCATCACCCGCTTTAAGCATCTCAAGAAGTTGCTGCTCGTGCATGGCCAC |
| | TGGTGTTACTCGCGCCTGGCCAGGATGGTGGTGTACTACCTCTACAAGAACGTGTGCT |
| | ACGTCAACCTGCTCTTCTGGTATCAGTTCTTCTGTGGTTTCTCCAGCTCCACCATGAT |
| | TGATTACTGGCAGATGATATTCTTCAATCTCTTCTTTACCTCCTTGCCTCCTCTTGTC |
| | TTTGGAGTCCTTGACAAAGACATCTCTGCAGAAACACTCCTGGCATTGCCTGAGCTAT |
| | ACAAGAGTGGCCAGAACTCTGAGTGCTATAACCTGTCGACTTTCTGGATTTCTATGGT |
| | GGATGCATTCTACCAGAGCCTCATCTGTTTCTTTATCCCTTACCTGGCCTATAAGGGC |
| | TCTGATATAGATGTCTTTACCTTTGGGACACCAATCAACACCATCTCCCTCACCACAA |
| | TCCTTTTGCACCAGGCAATGGAAATGAAGACATGGACCATTTTCCACGGAGTCGTGCT |
| | CCTCGGCAGCTTCCTGATGTACTTTCTGGTATCCCTCCTGTACAATGCCACCTGCGTC |
| | ATCTGCAACAGCCCCACCAATCCCTATTGGGTGATGGAAGGCCAGCTCTCAAACCCCA |
| | CTTTCTACCTCGTCTGCTTTCTCACACCAGTTGTTGCTCTTCTCCCAAGATACTTTTT |
| | CCTGTCTCTGCAAGGAACTTGTGGGAAGTCTCTAATCTCAAAAGCTCAGAAAATTGAC |
| | AAACTCCCCCCAGACAAAAGAAACCTGGAAATCCAGAGTTGGAGAAGCAGACAGAGGC |
| | CTGCCCCTGTCCCCGAAGTGGCTCGACCAACTCACCACCCAGTGTCATCTATCACAGG |
| | ACAGGACTTCAGTGCCAGCACCCCAAAGAGCTCTAACCCTCCCAAGAGGAAGCATGTG |
| | GAAGAGTCAGTACTCCACGAACAGAGATGTGGCACGGAGTGCATGAGGGATGACTCAT |
| | GCTCAGGGGACTCCTCAGCTCAACTCTCATCCGGGGAGCACCTGCTGGGACCTAACAG |
| | GATAATGGCCTACTCAAGAGGACAGACTGATATGTGCCGGTGCTCAAAGAGGAGCAGC |
| | CATCGCCGATCCCAGAGTTCACTGACCATATGAGGAGCTGCAGAAATCTGTACAAACT |
| | CAACAGAGGCCACCTAGTCACTGGTCCACATAACCCTTGACCCCTTCTTCTTCATAGA |
| | GGAAACAATGTGCCAGTCTTATTCTTTTCTTCAACAACCTTGACTTCCATGGAGGAAG |
| | TGCTGGCCCCAAGGGGTCTGACACAAAGACGGGAAACCCAGTCGGCCTCTAGTTTTCT |
| | GCTGCTCTCAGGCAGCACATCTTGCAAACAGTTTGGAGAAGGAGGCTGTTTTTGTTGA |
| | ATCGAGTTCTCAAATCGGTTTAGACCAAAGCCATTCTTCTGACCCTC |
| | ORF Start: ATG at 165 ORF Stop. TGA at 4497 |
| | SEQ ID NO: 132 1444 aa MW at 163004.1kD |
| NOV40a, | MRIKKTTLSRDMKKEGRKRWKRKEDKKRVVVSNLLFEGWSHKENPNRHHRGNQIKTSK |
| CG133819-01 | |
| Protein | YTVLSFVPKNIEEQLHRLANLYFVGIAVLNFIPVVNAFQPEVSMIPICVILAVTAIKD |
| Sequence | |
| | AWEDLRRYKSDKVINNRECLTYSRKEQTYVQKCWKDVRVGDFIQMKCNEIVPADILLL |
| | FSSDPNGICHLETASLDGETNLKQRRVVKGPSQQEVQFEPELFHNTIVCEKPNNHLNK |
| | FKGYMEHPDQTRTGFGCESLLLRGCTIRNTEMAVGIVIYAGHETKAMLNNSGPRYKRS |
| | KIERRMNIDIFFCIGTLILMCLIGAVGHSIWNGTFEEHPPFDVPDANGSFLPSALGGF |
| | YMFLTMITLLQVLIPISLYVSIELVKLGQVFFLSNDLDLYDEETDLSIQCRALNIAED |
| | LGQIQYIPSDKTGTLTENKMVFRRCTIMGSEYSHQENAKRLETPKELDSDGEEWTQYQ |
| | CLSFSARWAQDPATMRSQKGAQRLRRSQSARVPTQGHYRQRSMGHRESSQPPVAFSSS |
| | IEKDVTPDKNLLTKVRDAALWLETLSDSRPAKASLSTTSSIADFFLDLTTCNSVMVST |
| | TTEPRQRVTTKPSSKALGTSLEKIQQLFQKLKLLSLSQSFSSTAPSDTDLGESLGANV |
| | ATTDSDERDDASVCSGGDSTDDGGYRSSMWDQGDILESGSGTSLEEALEAPATDLARP |
| | EFCYEAESPDEAALVHAAHAYSFTLVSRTPEQVTVRLPQGTCLTFSLLCTLGPDSVRK |
| | RMSVVVRHPLTGEIVVYTKGADSVIMDLLEDPACVPDTNMEKKLRKIRARTQKHLDLY |
| | ARDGLRTLCIAKKVVSEEDFRRWASFRREAEASLDNRDELLMETAQHLENQLTLLGAT |
| | GIEDRLQEGVPDTTATLREAGIQLWVLTGDKQETAVNIAHSCRLLNQTDTVYTINTEN |
| | QETCESTLNCALEELKQFRELQKPDRKLPGFRLPSKTPSITSEAVVPEAGLVIDGKTL |
| | NAIFQGKLEKKFLELTQYCRSVLCCRSTPLQKSMTVKLVRDKLRVMTLSTGDGANDVS |
| | MIQAADIGIGISGQEGMQAVMSSDFATTRFKHLKKLLLVHGHWCYSRLARMVVYYLYK |
| | NVCYVNLLFWYQFFCGFSSSTMIDYWQMIFFNLPFTSLPPLVFGVLDKDISAETLLAL |
| | PELYKSGQNSECYNLSTFWISMVDAFYQSLTCFFIPYLAYKGSDIDVFTFGTPINTIS |
| | LTTILLHQAMEMKTWTIFHGVVLLGSFLMYFLVSLLYNATCVICNSPTNPYWVMEGQL |
| | SNPTFYLVCFLTPVVALLPRYFFL8LQGTCGKSLISKAQKIDKLPPDKRNLEIQSWRS |
| | RQRPAPVPEVARPTHHPVSSITGQDFSASTPKSSNPPKRKHVEESVLHEQRCGTECMR |
| | DDSCSGDSSAQLSSGEHLLGPNRTMAYSRGQTDMCRCSKRSSHRRSQSSLTI |

Further analysis of the NOV40a protein yielded the following properties shown in Table 40B.

TABLE 40B
Protein Sequence Properties NOV40a

| | |
|---|---|
| PSort | 0.6000 probability located in plasma membrane; 0.5165 |
| analysis: | probability located in mitochondrial inner membrane; |
| | 0.4000 probability located in Golgi body; 0.3200 |
| | probability located in nucleus |
| SignalP | No Known Signal Sequence Predicted |
| analysis: | |

A search of the NOV40a protein against the Geneseq database, a proprietary database that contains sequences published in patents and patent publication, yielded several homologous proteins shown in Table 40C.

TABLE 40C
Geneseq Results for NOV40a

| | | NOV40a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAE21185 | Human TRICH-29 protein-Homo | 14 . . . 1444 | 1313/1489 (88%) | 0.0 |
| | sapiens, 1519 aa. [WO200212340- | 34 . . . 1519 | 1356/1489 (90%) | |
| | A2, 14 Feb. 2002] | | | |
| AAE01984 | Human ATPase-related protein #7- | 52 . . . 1333 | 693/1296 (53%) | 0.0 |
| | Homo sapiens, 1426 aa. | 74 . . . 1351 | 916/1296 (70%) | |
| | [WO200134778-A2, 17 May 2001] | | | |
| AAE01982 | Human ATPase-related protein #5- | 52 . . . 1234 | 649/1197 (54%) | 0.0 |
| | Homo sapiens, 1270 aa. | 74 . . . 1252 | 849/1197 (70%) | |
| | [WO200134778-A2, 17 May 2001] | | | |
| AAU14142 | Human novel protein #13-Homo | 296 . . . 1375 | 545/1108 (49%) | 0.0 |
| | sapiens, 1194 aa. [WO200155437- | 1 . . . 1077 | 712/1108 (64%) | |
| | A2, 2 Aug. 2001] | | | |
| AAU14378 | Human novel protein #249-Homo | 296 . . . 1322 | 531/1039 (51%) | 0.0 |
| | sapiens, 1070 aa. [WO200155437- | 1 . . . 1010 | 689/1039 (66%) | |
| | A2, 2 Aug. 2001] | | | |

In a BLAST search of public sequence datbases, the NOV40a protein was found to have homology to the proteins shown in the BLASTP data in Table 40D.

TABLE 40D
Public BLASTP Results for NOV40a

| | | NOV40a | | |
|---|---|---|---|---|
| Protein | | Residues/ | Identities/ | |
| Accession | | Match | Similarities for | Expect |
| Number | Protein/Organism/Length | Residues | the Matched Portion | Value |
| O94823 | Potential phospholipid-transporting | 531 . . . 1444 | 913/914 (99%) | 0.0 |
| | ATPase VB (EC 3.6.3.1)-Homo | 1 . . . 914 | 913/914 (99%) | |
| | sapiens (Human), 914 aa (fragment). | | | |
| O54827 | Potential phospholipid-transporting | 9 . . . 1406 | 718/1445 (49%) | 0.0 |
| | ATPase VA (EC 3.6.3.1)-Mus | 13 . . . 1435 | 946/1445 (64%) | |
| | musculus (Mouse), 1508 aa. | | | |
| Q96914 | Putative aminophospholipid | 16 . . . 1375 | 713/1401 (50%) | 0.0 |
| | translocase (Aminophospholipid- | 15 . . . 1382 | 933/1401 (65%) | |
| | transporting ATPase)-Homo sapiens | | | |
| | (Human), 1499 aa. | | | |
| AAM20894 | P locus fat-associated ATPase-Mus | 141 . . . 1406 | 648/1300 (49%) | 0.0 |
| | musculus (Mouse), 1354 aa | 1 . . . 1281 | 854/1300 (64%) | |
| | (fragment). | | | |
| O60312 | Potential phospholipid-transporting | 326 . . . 1375 | 535/1077 (49%) | 0.0 |
| | ATPase VC (EC 3.6.3.1)-Homo | 1 . . . 1046 | 694/1077 (63%) | |
| | sapiens (Human), 1163 aa | | | |
| | (fragment). | | | |

PFam analysis predicts that the NOV40a protein contains the domains shown in the Table 40E.

TABLE 40E
Domain Analysis of NOV40a

| | | Identities/ | |
|---|---|---|---|
| Pfam | | Similarities for | Expect |
| Domain | NOV40a Match Region | the Matched Region | Value |
| Hydrolase | 410 . . . 1059 | 35/657 (5%) | 0.61 |
| | | 384/657 (58%) | |

### Example 41

The NOV41 clone was analyzed, and the nucleotide and encoded polypeptide sequences are shown in Table 41A.

TABLE 41A
NOV41 Sequence Analysis

| | |
|---|---|
| | SEQ ID NO: 133 569 bp |
| NOV41a, | TGCTTTGCAGATGCTGCCGTCGGGAGCTCTGTATTACCAGCCATGGTCAACCCCACCG |
| CG134375-01 | |
| DNA | TGTTCTTCCACATCTCTGTCGACGGTGAGTCCTTGGGCCGCATCTCTTTTGAGCTGTT |
| Sequence | |
| | TGCAGACAAGTTTCCAAAGACAGCAGAAAACTTTTGTGCTCTGAATACTGGAGAGAAA |
| | GGATTTGGTTACAAGGGTTGCTGCTTTCACAGAATTATTCCAGGGTTTATGTGTCATG |
| | GTGGTGACTTCACACACCATAATGGCACTGGTGGCAAGTCAATCTACGGGGAGAAAGT |
| | TGATGATGACAACTTCATCCTGAAGCATACAGGTCCTGGCATATTGTCCATGGCAAAT |
| | GCTGGACCCAACACAAATGGTTCCCAGTTTTTCATCTGCACTGCCAAGTCTGAGTGGT |
| | TGGATAGCAGCATGTGGTCATTGGCAAGGTGAGAAAGAAGCATGAATATTGTGGAGGC |
| | CATGGAGCACTTTGGGTCCAGGAATGGCAAGACCAGCAAGAAGGTCACCATTCCTGAC |
| | TTTGGACAACTCGAATAAGTTTGACTTGTGTTTTATCTTAACCACTG |
| | ORF Start: ATG at 43 ORF Stop: TAA at 538 |
| | SEQ ID NO: 134 165 aa MW at 18025.4kD |
| NOV41a, | MVNPTVFFHI8VDGESLGRISPELFADKFPKTAENFCALNTGEKGPGYKGCCFHRIIP |
| CG134375-01 | |
| Protein | GFMCHGGDFTHHNGTGGKSIYGEKVDDDNPILKHTGPGTLSMANAGPNTNGSQPFICT |
| Sequence | |
| | AKSFWLDSKHVVIGKVKEGMNIVEAMEHFGSRNGKTSKKVTIPDFGQLE |

Further analysis of the NOV41 a protein yielded the following properties shown in Table 41B.

TABLE 41B
Protein Sequence Properties NOV41a

| | |
|---|---|
| PSort | 0.6400 probability located in microbody (peroxisome); |
| analysis: | 0.4500 probability located in cytoplasm; 0.1000 probability |
| | located in mitochondrial matrix space; 0.1000 probability |
| | located in lysosome (lumen) |
| SignalP | No Known Signal Sequence Predicted |
| analysis: | |

A search of the NOV41at protein against the Geneseq database a proprietary database that contains sequences published in patents and patent publication, yielded several homologous proteins shown in Table 41C.

TABLE 41C
Geneseq Results for NOV41a

| | | NOV41a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAU01195 | Human cyclophilin A protein-Homo | 1 . . . 165 | 145/165 (87%) | 5e−84 |
| | sapiens, 165 aa. [WO200132876-A2, | 1 . . . 165 | 152/165 (91%) | |
| | 10 May 2001] | | | |
| AAW56028 | Calcineurin protein-Mammalia, 165 aa. | 1 . . . 165 | 145/165 (87%) | 5e−84 |
| | [WO9808956-A2, 5 Mar. 1998] | 1 . . . 165 | 152/165 (91%) | |
| AAG65275 | Haematopoietic stem cell proliferation | 2 . . . 165 | 144/164 (87%) | 2e−83 |
| | agent related human protein #2-Homo | 1 . . . 164 | 151/164 (91%) | |
| | sapiens, 164 aa. [JP2001163798-A, | | | |
| | 19 Jun. 2001] | | | |
| AAP90431 | Cyclophilin-Homo sapiens (human), | 2 . . . 165 | 144/164 (87%) | 2e−83 |
| | 164 aa. [EP326067-A, 2 Aug. 1989] | 1 . . . 164 | 151/164 (91%) | |
| AAG03831 | Human secreted protein, SEQ ID NO: | 1 . . . 165 | 144/165 (87%) | 3e−83 |
| | 7912-Homo sapiens, 165 aa. | 1 . . . 165 | 151/165 (91%) | |
| | [EP1033401-A2, 6 Sep. 2000] | | | |

In a BLAST search of public sequence datbases, the NOV41 a protein was found to have homology to the proteins shown in the BLASTP data in Table 41D.

TABLE 41D
Public BLASTP Results for NOV41a

| | | NOV41a | | |
|---|---|---|---|---|
| Protein | | Residues/ | Identities/ | |
| Accession | | Match | Similarities for | Expect |
| Number | Protein/Organism/Length | Residues | the Matched Portion | Value |
| CAC39529 | Sequence 26 from Patent WO0132876- | 1 . . . 165 | 145/165 (87%) | 1e−83 |
| | Homo sapiens (Human), 165 aa. | 1 . . . 165 | 152/165 (91%) | |
| Q9BRU4 | Peptidylprolyl isomerase A (cyclophilin | 1 . . . 165 | 144/165 (87%) | 4e−83 |
| | A)-Homo sapiens (Human), 163 aa. | 1 . . . 165 | 151/165 (91%) | |
| P05092 | Peptidyl-prolyl cis-trans isomerase A | 2 . . . 165 | 144/164 (87%) | 4e−83 |
| | (EC 5.2.1.8) (PPlase) (Rotamase) | 1 . . . 164 | 151/164 (91%) | |
| | (Cyclophilin A) (Cyclosporin A-binding | | | |
| | protein)-Homo sapiens (Human),, 164 | | | |
| | aa. | | | |
| P04374 | Peptidyl-prolyl cis-trans isomerase A | 2 . . . 164 | 143/163 (87%) | 1e−82 |
| | (EC 5.2.1.8) (PPlase) (Rotamase) | 1 . . . 163 | 150/163 (91%) | |
| | (Cyclophilin A) (Cyclosporin A-binding | | | |
| | protein)-Bos taurus (Bovine), and, 163 | | | |
| | aa. | | | |
| Q961X3 | Peptidylprolyl isomerase A (cyclophilin | 1 . . . 165 | 144/165 (87%) | 1e−82 |
| | A)-Homo sapiens (Human), 165 aa. | 1 . . . 165 | 151/165 (91%) | |

PFam analysis predicts that the NOV41 a protein contains the domains shown in the Table 41E.

TABLE 41E
Domain Analysis of NOV41a

| | | Identities/ | |
|---|---|---|---|
| Pfam | | Similarities for | Expect |
| Domain | NOV41a Match Region | the Matched Region | Value |
| pro_isomerase | 5 . . . 165 | 110/180 (61%) | 1.4e−93 |
| | | 144/180 (80%) | |

### Example 42

The NOV42 clone was analyzed, and the nucleotide and encoded polypeptide sequences are shown in Table 42A.

TABLE 42a
NOV42 Sequence Analysis

| | |
|---|---|
| | SEQ ID NO: 135 568 bp |
| NOV42a, | TAACCCATCTCCCTCACTCTTCCTGGGCACCACAGACATTCTCAAGTCCCCCCTGGAT |
| CG135546-01 | |
| DNA | GGGGGGCCCGGGCTGTGGCAAAGGGACACAGTGCAAGAATATGGCGACCAAGTACGGC |
| Sequence | |
| | TCTGCCATGTGGGGCTGGACCAGCTACTGAGACACAGAGGCTCAAAGGAGCACGCAGC |
| | GGGGCCGGCAGATCCGTGACATCACGCTGCAGGGGCTCCTGGTGCCCGCGGGCATCAT |
| | CCCAGATATGGTCAGTGACAACATGTTGTCCCGCCCGGAGAGCCGGGGCTTCCTCATC |
| | GATGGCTTTCCCCAGGAGGTGAAGCAGGCCATGGAGTTTGAGCGCATCGTGAGTGGCC |
| | CTGAAGTGTGGGTGTGGGTGGGCCAGGCCCCCAGCGTCGTCATCGTGTTTGACTGCTC |
| | CATGGAGACGATGCTCCGACGAGTGCTACACTGGGGCCAGGTGGAGCACCGGGCAGAC |
| | TCTTGACCTACCAGCGCAATAACCTGCTCTGAAACGTAGGTGCTCC |
| | ORF Start: ATG at 57 ORF Stop: TGA at 552 |
| | SEQ ID NO: 136 165 aa MW at 18653.3kD |
| NOV42a, | MGGPGCGKGTQCKNMATKYGFCHVGLDQLLRQEAQRSTQRGRQIRDITLQGLLVPAGI |
| CG135546-01 | |
| Protein | IPDMVSDNMLSRPESRGPLIDGFPQEVKQANEFERIVSGPEVWVWVGQARSVVIVFDC |
| Sequence | |
| | SMETMLRRVLHWGQVEHRADDSELAIHQRLDTHYTLCEPVLTYQRNNLL |

Further analysis of the NOV42a protein yielded the following properties shown in Fable 42B.

TABLE 42B
Protein Sequence Properties NOV42a

| | |
|---|---|
| PSort | 0.6500 probability located in cytoplasm; 0.2470 |
| analysis: | probability located in lysosome (lumen); 0.1000 |
| | probability located in mitochondrial matrix space; |
| | 0.0661 probability located in microbody (peroxisome) |
| SignalP | No Known Signal Sequence Predicted |
| analysis: | |

A search of the NOV42a protein against the Geneseq database, a proprietary database that contains sequences published in patents and patent publication, yielded several homologous proteins shown in Table 42C.

TABLE 42C
Geneseq Results for NOV42a

| | | NOV42a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAR10650 | Adenylate kinase-Sus scrofa, 194 aa. | 1 . . . 159 | 66/160 (41%) | 4e−30 |
| | [EP412526-A, 13 Feb. 1991] | 14 . . . 163 | 98/160 (61%) | |
| AAP93318 | Amino acid sequence of swine | 1 . . . 159 | 66/160 (41%) | 2e−29 |
| | adenylate kinase (SAK)-Sus scrofa, | 14 . . . 163 | 96/160 (59%) | |
| | 193 aa. [JP01051087-A, 27 Feb. 1989] | | | |
| AAU17301 | Novel signal transduction pathway | 1 . . . 159 | 66/160 (41%) | 4e−29 |
| | protein, Seq ID 866-Homo sapiens, 386 | 205 . . . 354 | 98/160 (61%) | |
| | aa. [WO200154733-A1, 2 Aug. 2001] | | | |
| AAU17300 | Novel signal transduction pathway | 1 . . .159 | 66/160 (41%) | 4e−29 |
| | protein, Seq ID 865-Homo sapiens, 245 | 65 . . . 214 | 98/160 (61%) | |
| | aa. [WO200154733-A1, 2 Aug. 2001] | | | |
| AAE11776 | Human kinase (PKIN)-10 protein- | 1 . . . 159 | 66/160 (41%) | 4e−29 |
| | Homo sapiens, 357 aa. [WO200181555- | 176 . . . 325 | 98/160 (61%) | |
| | A2, 1 Nov. 2001] | | | |

In a BLAST search of public sequence datbases, the NOV42a protein was found to have homology to the proteins shown in the BLASTP data in Table 42D.

TABLE 42D
Public BLASTP Results for NOV42a

| | | NOV42a | | |
|---|---|---|---|---|
| Protein | | Residues/ | Identities/ | |
| Accession | | Match | Similarities for | Expect |
| Number | Protein/Organism/Length | Residues | the Matched Portion | Value |
| P12115 | Adenylate kinase (EC 2.7.4.3) (ATP- | 1 . . . 159 | 66/160 (41%) | 2e−31 |
| | AMP transphosphorylase)-Cyprinus | 13 . . . 162 | 102/160 (63%) | |
| | carpio (Common carp), 193 aa. | | | |
| P05081 | Adenylate kinase isoenzymne 1 (EC | 1 . . . 16 | 66/162 (40%) | 2e−30 |
| | 2.7.4.3) (ATP-AMP transphosphorylase) | 15 . . . 166 | 103/162 (62%) | |
| | (AK1) (Myokinase)-Gallus gallus | | | |
| | (Chicken), 194 aa. | | | |
| Q920P5 | Adenylate kinase isozyme 5-Mus | 1 . . . 159 | 67/160 (41%) | 1e−29 |
| | musculus (Mouse), 193 aa. | 13 . . . 162 | 99/160 (61%) | |
| P00571 | Adenylate kinase isoenzyme 1 (EC | 1 . . . 159 | 66/160 (41%) | 1e−29 |
| | 2.7.4.3) (ATP-AMP transphosphorylase) | 14 . . . 163 | 98/160 (61%) | |
| | (AK1) (Myokinase)-Sus scrofa (Pig), | | | |
| | 194 aa. | | | |
| K1HUA | adenylate kinase (EC 2.7.4.3) 1 | 1 . . . 159 | 66/160 (41%) | 1e−29 |
| | (tentative sequence)-human, 194 aa. | 14 . . . 163 | 98/160 (61%) | |

PFam analysis predicts that the NOV42a protein contains the domains shown in the Table 42E.

TABLE 42E
Domain Analysis of NOV42a

| | | Identities/ | |
|---|---|---|---|
| Pfam | NOV42a | Similarities for | Expect |
| Domain | Match Region | the Matched Region | Value |
| adenylatekinase | 1 . . . 159 | 51/189 (27%) | 2.1e−25 |
| | | 110/189 (58%) | |

### Example 43

The NOV43 clone was analyzed, and the nucleotide and encoded polypeptide sequences are shown in Table 43A.

TABLE 43A
NOV43 Sequence Analysis

| | |
|---|---|
| | SEQ ID NO: 137 2876 bp |
| NOV43a, | TTTTTACCTGGAGTTGTATACTATGTGGACCGTTACTGGAAAGAATGGTTTTTTCATT |
| CG136321-01 | |
| DNA | TATGGAATCTTAGAGTTGGAAGGGATTTCAAGGTTAAAAGTTGTGCTCCTTGATATTG |
| Sequence | |
| | TAGGTGAAGAAATGGAGGCTCCAGGAGGTGATATGAGTAACCCACTGTCACAAGGCCA |
| | AACCTTTGAGGAAGCTGATAAGAATGGTGACGGCTTGCTGAATATTGAAGAGATACAT |
| | CAGCTGATGCATAAACTGAATGTTAATCTGCCCCGAAGAAAAGTCAGACAAATGTTTC |
| | AGGAAGCCGACACAGATGAGAATCAGGGAAACTTTGACATTTGAGAGTTCTGTGTTTT |
| | TTACAAAATGATGTCTTTGAGACGAGACCTTTATTTGTTACTTTTGAGCTACAGTGAC |
| | AAGAAAGATCACCTAACTGTGGAAGAACTGGCTCAGTTTTTGAAGGTGGAGCAAAAGA |
| | TGAATAATGTGACAACGGACTATTGTCTTGACATCATAAAGAAGTTTGAAGTTTCAGA |
| | AGAAAATAAGGTGAAAAATGTTCTTGGCATAGAAGGCTTCACGAACTTCATGCGTAGT |
| | CCTGCCTGTGACATATTTAACCCATTGCACCATGAAGTGTACCAAGACATGGATCAGC |
| | CCCTCTGCAACTACTACATTGCTTCCTCTCACAATACATACCTGACTGGAGACCAGCT |
| | CCTTTCTCAGTCCAAAGTGGATATGTATGCACGGGTGCTGCAAGAGGGCTGTCGCTGT |
| | GTGGAAGTTGACTGTTGGGATGGCCCAGATGGAGAGCCAGTAGTACATCATGGTTACA |
| | CTCTCACTTCAAAAATTCTCTTCAGAGATGTTGTGGAGACCATCAACAAGCATGCCTT |
| | TGTGAAGAATGAGTTTCCTGTTATATTGTCTATCGAGAATCACTGCAGTATCCAGCAG |
| | CAAGGAAGATTGCTCAGTACCTGAAAGGAATAATTCGGAGACAAACTGGACCTGTCAT |
| | CTGTTGATACAGGGGAGTGCAAGCAGCTTCCAAGCCCTCAAAGTTTGAAAGGCAAAAT |
| | TCTAGTGAAGGGTAAGAAGTTGCCTTATCACCTTGGGGATGATGCAGAGGAAGGGGAA |
| | GTTTCCGATGAGGACAGTGCAGATGAAATTGAAGACGAGTGCAAATTCAAGCTCCATT |
| | ATAGTAATGGGACCACTGAGCATCAGGTGGAATCTTTCATAAGGAAAAAACTGGAGTC |
| | ACTGTTAAAAGAATCTCAAATTCGAGATAAAGAAGATCCTGATAGTTTCACAGTGCGG |
| | GCACTACTGAAGGCCACGCATGAAGGCTTAAATGCACACCTGAAGCAGAGTCCAGATG |
| | TAAAGGAAAGTGGAAAGAAATCACATGGACGATCCCTCATGACCAACTTTGGAAAACA |
| | TAAGAAAACTACAAAATCACGGTCTAAATCTTACAGTACTGATGATGAGGAAGACACA |
| | CAGCAGAGTACTGGCAAGGAGGGTGGCCAGCTGTACAGATTGGGTCGCCGAAGGAAAA |
| | CCATGAAGCTCTGCCGAGAACTCTCTGATTTGGTTGTGTACACAAACTCCGTGGCCGC |
| | TCAGGACATTGTGGATGACGGAACCACAGGAAATGTGTTATCATTCAGTGAAACAAGA |
| | GCACATCAGGTTGTTCAGCAAAAATCAGAGCAGTTCATGATTTATAATCAAAAGCAAC |
| | TCACGAGGATTTACCCCTCTGCCTACCGCATTGATTCCAGTAACTTCAACCCTCTCCC |
| | CTACTGGAACGCAGGCTGCCAGCTAGTGGCACTGAATTATCAATCTGAAGGACGAATG |
| | ATGCAGTTAAACCGAGCCAAATTCAAGGCAAATGGCAATTGTGGCTATGTCCTCAAAC |
| | CCCAGCAAATGTGCAAAGGTACTTTCAACCCTTTCTCTGGTGACCCTCTTCCTGCCAA |
| | CCCCAAAAAGCAGCTCATCCTGAAAGTTATCAGTGGACAGCAACTCCCCAAACCTCCA |
| | GACTCCATGTTTGGAGATCGAGGCGAGATCATTGACCCTTTTGTTGAAGTTGAAATTA |
| | TTGGATTGCCAGTAGATTGTTGTAAAGATCAAACCCGTGTGGTAGATGACAATGGATT |
| | TAACCCTGTGTGGGAAGAAACACTGACATTTACAGTACACATGCCAGAAATAGCTTTG |
| | GTTCGGTTCCTTGTGTGGGATCACGATCCCATTGGACGAGACTTTGTTGGACAAAGAA |
| | CTGTGACCTTCAGCAGCTTAGTGCCTGGCTACCGGCATGTCTATTTGGAAGGACTGAC |
| | AGAAGCATCCATATTTGTACACATAACCATCAATGAAATCTATGGAAAGAACAGACAA |
| | CTCCAGGGTCTGAAGGGACTGTTCAATAAGAATCCTAGGCACAGTTCTTCAGAAAACA |
| | ATTCCCATTATGTACGGAAGCGATCCATTGGAGATAGTATTCTGCGACGCACAGCTAG |
| | CGCCCCAGCCAAAGGCAGGAAAAAGAGCAATGGGCTTCCAAGAAAAATGGTGGAGATA |
| | AAGGATTCTGTGTCCGAGGCCACAAGAGATCAAGATGGCGTGCTGAGGAGGACCACAC |
| | GCAGTTTGCAAGCACGCCCTGTCTCTATGCCTGTTGACAGAAACCTTCTGGGAGCTTT |
| | GTCGCTGCCTGTATCTGAAACAGCAAAAGACATTGAAGGAAAAGAAAACTCTCTAGAC |
| | TCTAGCTTTTGCAGGCCGACTGAGCAGGCTAAGCAGAAAAATGTGCAAGTGCCTTTCC |
| | CCAGACAGTTAGAATGTGTAATGAAGATGGAAATTTCCGAGACCTGAATCCCCAAACC |
| | CAGACTGATCTCTCTTCTCTTCTTGAATATAAAAGTAAGCTGGCAAGATTTAAAAAAC |
| | TGAACCCAAATAAATATTCATCATTTTTTTCTTC |
| | ORF Start: ATG at 23 ORF Stop: TGA at 2771 |
| | SEQ ID NO: 138 916 aa MW at 104019.2kD |
| NOV43a, | MWTVTGKNGFFIYGILELEGISRLKVVLLDIVGEEMEAPGGDMSNPLSQGQTFEEADK |
| CG136321-01 | |
| Protein | NGDGLLNIEEIHQLMHKLNVNLPRRKVRQMFQEADTDENQGTLTFEEFCVFYKMMSLR |
| Sequence | |
| | RDLYLLLLsYsDKKDHLTVEELAQFLKVEQKMNNVTTDYCLDIIKKFEVSEENKVKNV |
| | LGTEGPTNFMRSPACDIFNPLHHEVYQDMDQPLCNYYIASSHNTYLTGDQLLSQSKVD |
| | MYARVLQEGCRCVEVDCWDGPDGERVVHHGYTLTSKILFRDVVETTNKHAFVKNEFPV |
| | ILSIENHCSIQQQRKTAQYLKGIFGDKLDLSSVDTGECKQLPSPQSLKGKTLVKGKKL |
| | PYHLGDDAEEGEVSDEDSADEIEDECKFKLHYSNGTTEHQVESFIRKKLESLLKESQI |
| | RDKEDPDSFTVRALLKATHEGLNAHLKQSPDVKESGKKSHGRSLMTNFGKHKKTTKSR |
| | SKSYSTDDEEDTQQSTGKEGGQLYRLGRRRKTMKLCPELSDLVVYTNSVAAQDIVDDG |
| | TTGNVLSFSETRAHQVVQQKSEQFMIYNQKQLTRIYPSAYRIDSSNFNPLPYWNAGCQ |
| | LVALNYQSEGRMMQLNRAKPKANGNCGYVLKPQQMCKGTFNPFSGDRLPANPKKQLIL |
| | KVISGQQLPKPPDSMFGDRGEIIDPFVEVEIIGLPVDCCKDQTRVVDDNGFNPVWEET |
| | LTFTVHMPEIALVRFLVWDHDPIGRDFVGQRTVTFSSLVPGYRHVYLEGLTEASIFVH |
| | ITINEIYGKNRQLQGLKGLPNKNRRHSSSENNSHYVRKRSIGDRILRRTASAPAKGRK |
| | KSKMGFQEMVETKDSVSEATRDQDGVLRRTTRSLQARPVSMPVDRNLLGALSLPVSET |
| | AKDIEGKEN8LDSSFCRPTEQAKAEMCKVPFPRQLECVMKMEISET |

Further analysis of the NOV43a protein yielded the following properties shown in Table 43B.

TABLE 43B
Protein Sequence Properties NOV43a

| | |
|---|---|
| PSort | 0.9600 probability located in nucleus; 0.3000 probability |
| analysis: | located in microbody (peroxisome); 0.1000 probability located |
| | in mitochondrial matrix space; 0.1000 probability located in |
| | lysosome (lumen) |
| SignalP | No Known Signal Sequence Predicted |
| analysis: | |

A search of the NOV43a protein against the Geneseq database, a proprietary database that contains sequences published in patients and patent publication, yielded several homologous proteins shown in Table 43C.

TABLE 43C
Geneseq Results for NOV43a

| | | NOV43a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| ABG13669 | Novel human diagnostic protein #13660- | 118 . . . 881 | 764/784 (97%) | 0.0 |
| | Homo sapiens, 787 aa. [W0200175067- | 1 . . . 784 | 764/784 (97%) | |
| | A2, 11 Oct. 2001] | | | |
| ABG13669 | Novel human diagnostic protein #13660- | 118 . . . 881 | 764/784 (97%) | 0.0 |
| | Homo sapiens, 787 aa. [WO200175067- | 1 . . . 784 | 764/784 (97%) | |
| | A2, 11 Oct. 2001] | | | |
| ABB08205 | Human lipid metabolism enzyme-5 | 51 . . . 834 | 505/823 (61%) | 0.0 |
| | (LME-5)-Homo sapiens, 1239 aa. | 171 . . . 989 | 623/823 (75%) | |
| | [WO200185956-A2, 15 Nov. 2001] | | | |
| AAB95125 | Human protein sequence SEQ ID | 451 . . . 916 | 466/466 (100%) | 0.0 |
| | NO: 17124-Homo sapiens, 466 aa. | 1 . . . 466 | 466/466 (100%) | |
| | [EP1074617-A2, 7 Feb. 2001] | | | |
| ABB07493 | Human lipid metabolism molecule | 51 . . . 481 | 271/433 (62%) | e−157 |
| | (LMM) polypeptide (ID: 2965233CD1)- | 173 . . . 604 | 340/433 (77%) | |
| | Homo sapiens, 621 aa. [WO200204490- | | | |
| | A2, 17 Jan. 2002] | | | |

In a BLAST search of public sequence datbases, the NOV43a protein was found to have homology to the proteins shown in the BLASTP data in Table 43D.

TABLE 43D
Public BLASTP Results for NOV43a

| | | NOV43a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9UPT3 | KIAA1069 protein-Homo sapiens | 118 . . . 881 | 764/784 (97%) | 0.0 |
| | (Human), 787 aa (fragment). | 1 . . . 784 | 764/784 (97%) | |
| Q9H9U2 | CDNA FLJ12548 fis, clone | 451 . . . 916 | 466/466 (100%) | 0.0 |
| | NT2RM4000657, weakly similar to 1- | 1 . . . 466 | 466/466 (100%) | |
| | phosphatidylinositol-4,5-bisphosphate | | | |
| | phosphodiesterase delta 1 (EC 3.1.4.11)- | | | |
| | Homo sapiens (Human), 466 aa. | | | |
| Q8TEH5 | FLJ00222 protein-Homo sapiens | 444 . . . 834 | 243/395 (61%) | e−138 |
| | (Human), 656 aa (fragment). | 15 . . . 406 | 304/395 (76%) | |
| Q8WUS6 | Hypothetical 75.7 kDa protein-Homo | 577 . . . 834 | 179/261 (68%) | e−101 |
| | sapiens (Human), 716 aa (fragment). | 1. . . 261 | 211/261 (80%) | |
| Q91UZ1 | Phospholipase C beta 4-Mus musculus | 102 . . . 757 | 933/687 (33%) | 4e−89 |
| | (Mouse), 1175 aa. | 205 . . . 820 | 351/687 (50%) | |

PFam analysis predicts that the NOV43a protein contains the domains shown in the Table 43E.

TABLE 43E
Domain Analysis of NOV43a

| | | Identities/ | |
|---|---|---|---|
| Pfam | NOV43a | Similarities for | Expect |
| Domain | Match Region | the Matched Region | Value |
| efhand | 48 . . . 26 | 11/29 (38%) | 0.016 |
| | | 22/29 (76%) | |
| RrnaAD | 76 . . . 111 | 6/42 (14%) | 0.13 |
| | | 27/42 (64%) | |
| efhand | 84 . . . 113 | 10/30 (33%) | 0.027 |
| | | 27/30 (90%) | |
| PI-PLC-X | 202 . . . 347 | 80/153 (52%) | 1.1e−68 |
| | | 122/153 (80%) | |
| PI-PLC-Y | 502 . . . 616 | 50/128 (39%) | 6.8e−42 |
| | | 82/128 (64%) | |
| C2 | 636 . . . 728 | 38/102 (37%) | 1.8e−27 |
| | | 78/102 (76%) | |

### Example 44

The NOV44 clone was analyzed, and the nucleotide and encoded polypeptide sequences arc shown in Table 44A.

TABLE 44A
NOV44 Sequence Analysis

| | |
|---|---|
| | SEQ ID NO: 139 1742 bp |
| NOV44a, | TAATTTAAACCAGTGTTTGTGCGGTTCTGATTCATCTGCTGTGGTTCCCGAAGCTTGA |
| CG136648-01 | |
| DNA | GATCTAAGGAGTACAGGGTCTTTTGTGATGACAATATGACTAATAGTAAAGGAAGATC |
| Sequence | |
| | TATTACCGATAAAACAAGTGGTGGTCCAAGTAGTGGAGGAGCTTTTGTAGATTGGACT |
| | TTACGTTTAAACACAATTCAATCCGACAAGTTTTTAAATTTACTCTTGAGTATGGTTC |
| | CAGTGATTTACCAGAAAAACCAAGAAGACAGGCACAAAAAAGCAAACGGCATTTGGCA |
| | AGATGGATATCAACTGCAGTACAGACTTTTAGTAATAGATCTGAGCAACACATGGAGT |
| | ATCACAGTTTCTCAGAGCAGTCTTTTCATGCCAATAATGGGCACGCATCATCAAGCTG |
| | CAGCCAAAAGTATGATGACTATGCCAATTGTAATTACTGTGATGGAAGGGAGACTTCA |
| | GAAACCACTGCCATGTTACAAGATGAAGATATATCTAGTGATGGTGATGAAGATGCTA |
| | TTGTAGAAGTGACCCCAAAATTACCAAAGGAATCCAGTGGCATCATGGCATTGCAAAT |
| | ACTTGTGCCCTTTTTGCTAGCTCGTTTTGGAACAGTTTCAGCTGGCATGGTACTGGAT |
| | ATAGTACAGCACTGGGAGGTGTTCAGAAAAGTTACAGAAGTTTTCATTTTAGTCCCTG |
| | CACTTCTTGGTCTCAAAGGGAACTTGGAAATGACATTGGCATCCAGATTATCCACTGC |
| | AGTAAATATTGGGAAGATGGATTCACCCATTGAAAAGTGGAACCTAATAATTGGCAAC |
| | TTGGCTTTAAAGCAGGGAATAATAATGGTTGGGGTTATCGTTGGTTCAAAGAAGACTG |
| | GTATAAATCCTGATAATGTTGCTACACCCATTGCTGCTAGTTTTGGCGACCTTATAAC |
| | TCTTGCCATATTGGCTTGGATAAGTCAGGGCTTATACTCCTGTCTTGAGACCTATTAC |
| | TACATTTCTCCATTAGTTGGTGTATTTTTCTTGGCTCTAACCCCTATTTGGATTATAA |
| | TAGCTGCCAAACATCCAGCCACAAGAACAGTTCTCCACTCAGGCTGGGAGCCTGTCAT |
| | AACAGCTATGGTTATAAGTAGCATTGGGGGCCTTATTCTGGACACAACTGTATCAGAC |
| | CCAAACTTGGTTGGGATTGTTGTTTACACGCCAGTTATTAATGGTATTGGTGGTAATT |
| | TGGTGGCCATTCAGGCTAGCAGGATTTCTACCTACCTCCATTTACATAGCATTCCAGG |
| | AGAATTGCCTGATGAACCCAPAGGTTGTTACTACCCATTTAGAACTTTCTTTGGTCCA |
| | GGAGTAAATAATAAGTCTGCTCAAGTTCTACTGCTTTTAGTGATTCCTGGACATTTAA |
| | TTTTCCTCTACACTATTCATTTGATGAAAACTGGTCATACTTCTTTAACTATAATCTT |
| | CATAGTAGTGTATTTATTTGGCGCTGTGTTACAGGTATTTACCTTGCTGTGGATTGCT |
| | GACTGGATGGTCCATCACTTCTGGAGGAAAGGAAAGGACCCGGATAGTTTCTCCATCC |
| | CCTACCTAACAGCATTGGGTGATCTGCTCGGGACAGCTCTGTTAGCCTTAAGTTTTCA |
| | TTTTCTTTGGCTTATTGGAGATCGAGATGGAGATGTTGGAGACTAATAAATTCTACAA |
| | ACTGCTCTCAAGTTACCAAGGAAGAAAATACACGACAACCACTTATCGCTCTTTTTCA |
| | AA |
| | ORF Start: ATG at 342 ORF Stop: TAA at 1668 |
| | SEQ ID NO: 140 442 aa MW at 48201.3kD |
| NOV44a, | MEYHSFSEQSFHANNGHASSSCSQKYDDYANCNYCDGRETSETTAMLQDEDISSDGDE |
| CG136648-01 | |
| Protein | DAIVEVTPKLPKESSGTMALQILVPFLLAGFGTVSAGMVLDIVQHWEVFRKVTEVFIL |
| Sequence | |
| | VPALLGLKGNLEMTLASRLSTAVNIGKMDSPIEKWNLITGNLALKQGITMVGVIVGSK |
| | KTGINPDNVATPIAASFGDLITLAILAWISQGLYSCLETYYYISPLVGVFFLALTPIW |
| | IIIAAKHPATRTVLHSGWEPVTTAMVISSTGGLILDTTVSDPNLVGIVVYTPVINGIG |
| | GNLVAIQASRTSTYLHLHSIPGELPDEPKGCYYPFRTFFGPGVNNKSAQVLLLLVIPG |
| | HLIFLYTIHLMKSGHTSLTIIFIVVYLFGAVLQVFTLLWTADWMVHHFWRKGKDPDSF |
| | SIPYLTALGDLLGTALLALSFHFLWLIGDRDGDVGD |

Further analysis of the NOV44a protein yielded the following properties shown in Table 44B.

TABLE 44B
Protein Sequence Properties NOV44a

| | |
|---|---|
| PSort | 0.6000 probability located in plasma membrane; 0.4000 |
| analysis: | probability located in Golgi body: 0.3000 probability |
| | located in endoplasmic reticulum (membrane); 0.3000 |
| | probability located in microbody (peroxisome) |
| SignalP | No Known Signal Sequence Predicted |
| analysis: | |

A search of the NOV44a protein against the Geneseq database, a proprietary database that contains sequences published in patents and patent publication, yielded several homologous proteins shown in Table 44C.

TABLE 44C
Geneseq Results for NOV44a

| | | NOV44a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAB95482 | Human protein sequence SEQ ID | 1 . . . 442 | 442/490 (90%) | 0.0 |
| | NO: 18007-Homo sapiens, 490 aa | 1 . . . 490 | 442/490 (90%) | |
| | [EP1074617-A2, 7 Feb. 2001] | | | |
| ABB08638 | Human transporter protein SEQ ID NO | 42 . . . 442 | 274/453 (60%) | e−148 |
| | 2-Homo sapiens, 513 aa. | 62 . . . 513 | 328/453 (71%) | |
| | [WO200190360-A2, 29 Nov. 2001] | | | |
| AAM47910 | Human initiation factor 46-Homo | 85 . . . 433 | 189/398 (47%) | 1e−92 |
| | sapiens, 414 aa. [CN1307045-A, | 1 . . . 397 | 246/398 (61%) | |
| | 8 Aug. 2001] | | | |
| AAB93857 | Human protein sequence SEQ ID | 64 . . . 433 | 172/382 (45%) | 7e−78 |
| | NO: 13719-Homo sapiens, 438 aa. | 48 . . . 421 | 233/382 (60%) | |
| | [EP1074617-A2, 7 Feb. 2001] | | | |
| AAB94260 | Human protein sequence SEQ ID | 64 . . . 421 | 165/376 (43%) | 2e−75 |
| | NO: 14667-Homo sapiens, 464 aa. | 48 . . . 422 | 228/376 (59%) | |
| | [EP1074617-A2, 7 Feb. 2001] | | | |

In a BLAST search of public sequence datbases, the NOV44a protein was found to have homology to the proteins shown in the BLASTP data in Table 44D.

TABLE 44D
Public BLASTP Results for NOV44a

| | | NOV44a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q96JW4 | CDNA FLJ14932 fis, clone | 1 . . . 442 | 442/490 (90%) | 0.0 |
| | PLACE1009639-Homo sapiens | 1 . . . 490 | 442/490 (90%) | |
| | (Human), 490 aa. | | | |
| Q9H0E5 | Hypothetical 53.3 kDa protein- | 1 . . . 442 | 441/490 (90%) | 0.0 |
| | Homo sapiens (Human), 490 aa. | 1 . . . 490 | 441/490 (90%) | |
| Q9HAB1 | Hypothetical 47.2 kDa protein- | 64 . . . 433 | 172/382 (45%) | 2e−77 |
| | Homo sapiens (Human), 438 aa. | 48 . . . 421 | 233/382 (60%) | |
| Q9H9I6 | CDNA FLJ12718 fis, clone | 64 . . . 421 | 165/376 (43%) | 5e−75 |
| | NT2RP1001286-Homo sapiens | 48 . . . 422 | 228/376 (59%) | |
| | (Human), 464 aa. | | | |
| Q9NX30 | CDNA FLJ20473 fis, clone | 64 . . . 421 | 165/376 (43%) | 5e−75 |
| | KAT07092-Homo sapiens | 48 . . . 422 | 228/376 (59%) | |
| | (Human), 471 aa. | | | |

PFam analysis predicts that the NOV44a protein contains the domains shown in the Table 44E.

TABLE 44E
Domain Analysis of NOV44a

| | | Identities/ | |
|---|---|---|---|
| Pfam | NOV44a | Similarities for | Expect |
| Domain | Match Region | the Matched Region | Value |
| MgtE | 116 . . . 204 | 29/137 (21%) | 3.2e−06 |
| | | 77/137 (56%) | |
| MgtE | 282 . . . 428 | 31/153 (20%) | 7.4e−07 |
| | | 106/153 (69%) | |

### Example 45

The NOV45 clone was analyzed, and the nucleotide and encoded polypeptide sequences are shown in Table 45A.

TABLE 45A
NOV45 Sequence Analysis

| | |
|---|---|
| | SEQ ID NO 141 2200 bp |
| NOV45a | TGCAGCCTCCAGCCAGAAGGATGGGGTGGCTCCCACTCCTGCTGCTTCTGACTCAATG |
| CG54479-01 | |
| DNA | CTTAGGGGTCCCTGGGCAGCGCTCGCCATTGAATGACTTCGAGGTGCTCCGGGGCACA |
| Sequence | |
| | GAGCTACAGCGGCTGCTACAAGCGGTGGTGCCCGGGCCTTGGCAGGAGGATGTGGCAG |
| | ATGCTGAAGAGTGTGCTGGTCGCTGTGGGCCCTTAATGGACTGCCGGGCGTTCCACTA |
| | CAATGTGAGCAGCCATGGTTGCCAACTGCTGCCATGGACTCAACACTCACCCCACACG |
| | AGGCTGCGGCATTCTGGGCGCTGTGACCTCTTCCAGGAGAAAGACTACATACGGACCT |
| | GCATCATGAACAATGGGGTTGGGTACCGGGGCACCATGGCCACGACCGTGGGTGGCCT |
| | GTCCTGCCAGGCTTGGAGCCACAAGTTCCCGAACGATCACAGGTACATGCCCACGCTC |
| | CGGAATGGCCTGGAAGAGAACTTCTGCCGTAACCCTGATGGCGACCCCGGAGGTCCTT |
| | GGTGCCACACAACAGACCCTCCCGTGCGCTTCCAGAGCTGCGGCATCAAATCCTGCCG |
| | GTCTGCCGCGTGTGTCTGGTGCAATGGCGAGGAATACCGCGGCGCGGTAGACCGCACC |
| | GAGTCAGGGCGCGAGTGCCAGCGCTGGGATCTTCAGCACCCGCACCAGCACCCCTTCG |
| | AGCCGGGCAAGTACCCCCACCAAGGTCTGGACGACAACTATTGCCGGAATCCTGACGG |
| | CTCCGAGCGGCCATGGTGCTACACTACGGATCCGCAGATCGAGCGAGAATTCTGTGAC |
| | CTCCCCCGCTGCGGTTCCGAGGCACAGCCCCGCCAAGAGGCCACAAGTGTCAGCTGCT |
| | TCCGCGGGAAGGGTGAGGGCTACCGGGGCACAGCCAATACCACCACCGCGGGCGTACC |
| | TTGCCAGCGTTGGGACGCGCAAATCCCGCATCAGCACCGATTTACGCCAGAAAAATAC |
| | GCGTGCAAGGACCTTCGGGAGAACTTCTGCCGGAACCCCGACGGCTCAGAGGCGCCCT |
| | GGTGCTTCACACTGCGGCCCGGCATGCGCGTGGGCTTTTGCTACCAGATCCGGCGTTG |
| | TACAGACGACGTGCGGCCCCAGGGTTGCTACCACGGCGCGGGGGAGCAGTACCGCGGC |
| | ACGGTCAGCAAGACCCGCAAGGGTGTCCAGTGCCAGCGCGCGTCCGCTGAGACGCCGC |
| | ACAAGCCGCAGTTTACCTTTACCTCCGAACCGCATGCACAACTGGAGGAGAACTTCTG |
| | CCGCGACCCAGATGGGGATAGCTATGGGCCCTGGTGCTACACGATGGACCCAAGGACC |
| | CCATTCGACTACTGTGCCCTGCGACGCTGCGCTGATGACCAGCCGCCATCAATCCTGG |
| | ACCCCCCCGACCAGGTGCACTTTGAGAAGTGTGGCAAGAGGGTGGATCGGCTGGATCA |
| | GCGTTGTTCCAAGCTGCGCGTGGCTGGGGGCCATCCGGGCAACTCACCCTGGACAGTC |
| | AGCTTGCGGAATAGGCAGGGCCAGCATTTCTGCGGGGGGTCTCTAGTGAAGGAGCAGT |
| | GGATACTGACTGCCCGGCAGTGCTTCTCCTCCAGCCATATGCCTCTCACGGGCTATGA |
| | GGTATGGTTGGGCACCCTGTTCCAGAACCCACAACATGGAGAGCCAGGCCTACAGCGG |
| | GTCCCAGTAGCCAAGATGCTGTGTGGGCCCTCAGGCTCTCAGCTTGTCCTGCTCAAGC |
| | TGGAGAGATCTGTGACCCTGAACCAGCGTGTGGCCCTGATCTGCCTGCCGCCTGAATG |
| | GTATGTGGTGCCTCCAGGGACCAAGTGTGAGATTGCAGGCCGGGGTGAGACCAAAGGT |
| | ACGGGTAATGACACAGTCCTAAATGTGGCCTTGCTGAATGTCATCTCCAACCAGGAGT |
| | GTAACATCAAGCACCGAGGACATGTGCGGGAGAGCGAGATGTGCACTGAGGGACTGTT |
| | GGCCCCTGTGGGGGCCTGTGAGGGGGGTGACTACGGGGGCCCACTTGCCTGCTTTACC |
| | CACAACTGCTGGGTCCTGGAAGGAATTAGAATCCCCAACCGAGTATGCGCAAGGTCGC |
| | GCTGGCCAGCCGTCTTCACACGTGTCTCTGTGTTTGTGGACTGGATTCACAAGGTCAT |
| | GAGACTGGGTTAGGCCCAGCCTTGACGCCATATGCTTTGGGGAGGACAAAACTT |
| | ORF Start: ATG at 21 ORF Stop: TAG at 2157 |
| | SEQ ID NO: 142 712 aa MW at 80097.8kD |
| NOV45a, | MGWLPLLLLLTQCLGVPGQRSPLNDFEVLRGTELQRLLQAVVPGPWQEDVADAEECAG |
| CG54479-01 | |
| Protein | RCGPLMDCRAFHYNVSSHGCQLLRWTQHSPHTRLRHSGRCDLFQEKDYIRTCIMNNGV |
| Sequence | |
| | GYRGTMATTVGGLSCQAWSHKFPNDHRYMPTLRNGLEENFCRNPDGDPGGPWCHTTDP |
| | AVRFQSCGIKSCRSAACVWCNGEEYRGAVDRTESGRECQRWDLQHPHQHPFEPGKYPD |
| | QGLDDNYCRNPDGSERPWCYTTDPQIEREFCDLPRCGSEAQPRQEATSVSCFRGKGEG |
| | YRGTANTTTAGVPCQRWDAQIPHQHRFTPEKYACKDLRENFCRNPDGSEAPWCFTLRP |
| | GMRVGFCYQTRRCTDDVRPQGCYHGAGEQYRGTVSKTRKGVQCQRASAETPHKPQFTF |
| | TSEPHAQLEENFCRDPDGDSYGPWCYTMDPRTPFDYCALRRCADDQPPSILDPPDQVQ |
| | FEKCGKRVDRLDQRCSKLRVAGGHPGNSPWTVSLRNRQGQHPCGGSLVKEQWILTARQ |
| | CFSSSHMPLTGYEVWLGTLFQNPQHGEPGLQRVPVAKMLCGPSGSQLVLLKLERSVTL |
| | NQRVALICLPPEWYVVPPGTKCETAGRGETKGTGNDTVLNVALLNVTSNQECNTKHRG |
| | HVRESEMCTEGLLAPVGACEGGDYGGPLACFTHNCWVLEGTRIPNRVCARSRWPAVFT |
| | RVSVPVDWTHKVMRLG |
| | SEQ ID NO: 143 1710 bp |
| NOV45b, | ATGACTTCCAGGTGCTCCGGGGCACAGAGCTACCTGCTACATGCGGTGGTGCCTGGGC |
| CG54479-02 | |
| DNA | CTTGGCAGGAGGATGTGGCAGATGCTGAAGAGTGTGCTGGTCGCTGTGGGCCCTTAAC |
| Sequence | |
| | GGACTGCTGGGCCTTCCACTACAATGTGAGCAGCCATGGTTGCCAACTGCTGCCATGG |
| | ACTCAACACTCGCCCCACTCAAGGCTGTGGCATTCTGGGCGCTGTGACCTCTTCCAGA |
| | AGAAAGACTACATACGGACCTGCATCATGAACAATGGGGTTGGGTACCGGGGCACCAT |
| | GGCCACGACCGTGGGTGGCCTGTCCTGCCAGGCTTGGAGCCACAAGTTCCCGAATGAT |
| | CACAAGTACATGCCCACGCTCCGGAATGGCCTGGAAGAGAACTTCTGCCATAACCCTG |
| | ATGGCGACCCCGGAGGTCCTTGGTGCCACACAACAGACCCTGCCGTGCGCTTCCAGAG |
| | CTGCGGCATCAAATCCTGCCGGGTGGCCGCGTGTGTCTGGTGCAATGGCGAGGAATAC |
| | CGCGGCGCGGTAGACCGCACCGAGTCAGGGCGCGAGTGCCAGCGCTGGGATCTTCAGC |
| | ACCCGCACCAGCACCCCTTCGAGCCGGGCAAGTACCTCGACCAAGGTCTGGACGACAA |
| | CTATTGCCGGAATCCTGACGGCTCCGAGCGGCCATGGTGCTACACTACGGATCCGCAG |
| | ATCGAGCGAGAATTCTGTGACCTCCCCCGCTGCGGTTCCGAGGCACAGCCCCGCCAAG |
| | AGGCCACAAGTGTCAGCTGCTTCCGCGGGAAGGGTGAGGGCTACCGGGGCACAGCCAA |
| | TACCACCACCGCGGGCGTACCTTGCCAGCGTTGGGACGCGCAAATCCCGCATCAGCAC |
| | CGATTTACGCCAGAAAAATACGCGTGCAAGGACCTTCGGGAGAACTTCTGCCGGAACC |
| | CCGACGGCTCAGAGGCGCCCTGGTGCTTCACACTGCGGCCCGGCATGCGCGTGGGCTT |
| | TTGCTACCAGATCCGGCGTTGTACAGACGACGTGCGGCCCCAGGACTGCTACCACGGC |
| | GCGGGGGAGCAGTACCGCGGCACGGTCAGCAAGACCCGCAAGGGTGTCCAGTGCCAGC |
| | GCGCGTCCGCTGAGACGCCGCACAAGCCGCAGTTCACGTTTACCTCCGAACCGCATGC |
| | ACAACTGGAGGAGAACTTCTGCCAGGACCCAGATGGGGATAGCCATGGGCCCTGGTGC |
| | TACACGATGGACCCAAGGACCCCATTCGACTACTGTGCCCTGCGACGCTGCGCTGATG |
| | ACCAGCCGCCATCAATCCTGGACCCCCCCACAGACCAGGTGCAGTTTGAGAAGTGTGG |
| | CAAGAGGGTGGATCGGCTGGATCAGCGTCGTTCCAAGCTGCGCGTGGCTGGGGGCCAT |
| | CCGGGCAACTCACCCTGGACAGTCAGCTTGGGGAATCGGAGGCAGGGCCAGCATTTCT |
| | GCGGGGGGTCTCTAGTGAAGGAGCAGTGGATACTGACTGCCCGGCAGTGCTTCTCCTC |
| | CCATATGCCTCTCACGGGCTATGAGGTATGGTTGGGCACCCTGTTCCAGAACCCACAA |
| | CATGGAGAGCCAGGCCTACAGCGGGTCCCAGTAGCCAAGATGCTGTGTGGGCCCTCAG |
| | GCTCCCAGCTTGTCCTGCTCAAGCTGGAGAGATCTGTGACCCTGAACCAGCGTGTGGC |
| | CCTGATCTGCCTGCCGCCTGAATGATAT |
| | ORF Start: ATG at 1 ORF Stop: TGA at 1705 |
| | SEQ ID NO: 144 568 aa MW at 64180.3kD |
| NOV45b, | MTSRCSGAQSYLLHAVVPGPWQEDVADAEFCAGRCGPLTDCWAPHYNVSSHGCQLLPW |
| CG54479-02 | |
| Protein | TQHSPHSRLWHSGRCDLFQKKDYTRTCIMNNGVGYRGTMATTVGGLSCQAWSHKFPND |
| Sequence | |
| | HKYMPTLRNGLEENFCHNPDGDRGGPWCHTTDRAVRFQ8CGIKSCRVAACVWCNGEEY |
| | RGAVDRTESGRECQRWDLQHPHQHPFEPGKYLDQGLDDNYCRNPDGSERPWCYTTDPQ |
| | IEREFCDLPRCGSEAQPRQEATSVSCFRGKGEGYRGTANTTTAGVPCQRWDAQIPHQH |
| | RFTPEKYACKDLRENFCRNPDGSEAPWCFTLRPGMRVGFCYQIRRCTDDVRPQDCYHG |
| | AGEQYRGTVSKTRKGVQCQRASAETPHKPQFTFTSEPHAQLEENFCQDPDGDSHGPWC |
| | YTMDPRTRFDYCALRRCADDQPPSILDPPTDQVQFEKCGKRVDRLDQRRSKLRVAGGH |
| | PGNSPWTVSLGNRRQGQHFCGGSLVKEQWILTARQCFSSHMPLTGYEVWLGTLFQNPQ |
| | HGEPGLQRVPVAKMLCGPSGSQLVLLKLERSVTLNQRVALICLPPE |
| | SEQ ID NO: 145 1011 bp |
| NOV45c, | AAGCTTTGCATCATGAACAATGGGGTTGGGTACCGGGGCACCATGGCCACGACCGTGG |
| CG54479-03 | |
| DNA | GTGGCCTGCCCTGCCAGGCTTGGAGCCACAAGTTCCCAAATGATCACAAGTACACGCC |
| Sequence | |
| | CACTCTCCGGAATGGCCTGGAAGAGAACTTCTGCCGTAACCCTGATGGCGACCCCGGA |
| | GGTCCTTGGTGCTACACAACAGACCCTGCTGTGCGCTTCCAGAGCTGCGGCATCGAAT |
| | CCTGCCGGGAGGCCGCGTGTGTCTGGTGCAATGGCGAGGAATACCGCGGCGCGGTAGA |
| | CCGCACGGAGTCAGGGCGCGAGTGCCAGCGCTGGGATCTTCAGCACCCGCACCAGCAC |
| | CCCTTCGAGCCGGGCAAGTTCCTCGACCAAGGTCTGGACGACAACTATTGCCGGAATC |
| | CTGACGGCTCCGAGCGGCCATGGTGCTACACTACGGATCCGCAGATCGAGCGAGAGTT |
| | CTGTGACCTCCCCCGCTGCGGGTCCGAGGCACAGCCCCGCCAAGAGGCCACAACTGTC |
| | AGCTGCTTCCGCGGGAAGGGTGAGGGCTACCGGGGCACAGCCAATACCACCACTGCCG |
| | GCGTACCTTGCCAGCGTTGGGACGCGCAPATCCCTCATCAGCACCGATTTACGCCAGA |
| | AAAATACGCGTGCAAAGACCTTCGGGAGAACTTCTGCCGGAACCCCGACGGCTCAGAG |
| | GCGCCCTGGTGCTTCACACTGCGGCCCGGCATGCGCGCGGCCTTTTGCTACCAGATCC |
| | GGCGTTGTACAGACGACGTGCGGCCCCAGGGGGAGCAGTACCGCGGCACGGTCAGCAA |
| | GACCCGCAAGGGTGTCCAGTGCCAGCGCTGGTCCGCTGAGACGCCGCACAAGCCGCAG |
| | TTCACGTTTACCTCCGAiCCGCATGCACAACTGGAGGAGAACTTCTGCCGGAACCCAG |
| | ATGGGGATAGCCATGGGCCCTGGTGCTACACGATGGACCCAAGGACCCCATTCGACTA |
| | CTGTGCCCTGCGACGCTGCCTCGAG |
| | ORF Start: at 7 ORF Stop: at 1006 |
| | SEQ ID NO: 146 333 aa MW at 38129.9kD |
| NOV45c, | CIMNNGVGYRGTMATTVGGLPCQAWSHKFPNDHKYTPTLRNGLEENFCRNPDGDPGGR |
| CG54479-03 | |
| Protein | WCYTTDPAVRFQSCGIESCREAACVWCNGEEYRGAVDRTESGRECQRWDLQHPHQHPF |
| Sequence | |
| | EPGKFLDQGLDDNYCRNPDGSERPWCYTTDPQIEREFCDLRRCGSEAQPRQEATTVSC |
| | FRGKGFGYRGTANTTTAGVPCQRWDAQIPHQHRFTPEKYACKDLRENFCRNPDGSEAP |
| | WCFTLRPGMRAAFCYQIRRCTDDVRRQGEQYRGTVSKTRKGVQCQRWSAETPHKPQFT |
| | FTSEPHAQLEENFCRNPDGDSHGPWCYTMDPRTPFDYCALRRC |
| | SEQ ID NO: 147 1881 bp |
| N0V45d, | ACACATTACTGACATGTATGCCCACCTGACCTGCACCCACTCATGCCCACTCTGCAGG |
| CG54479-04 | |
| DNA | GCAGCCCTCGCCATTGAATGACTTCCAGGTGCTCCGGGGCACAGAGCTACCTGCTACA |
| Sequence | |
| | TGCGGTGGTGCCTGGGCCTTGGCAGGAGGATGTGGCAGATGCTGAAGAGTGTGCTGGT |
| | CGCTGTGGGCCCTTAACGGACTGCTGGGCCTTCCACTACAATGTGAGCAGCCATGGTT |
| | GCCAACTGCTGCCATGGACTCAACACTCGCCCCACTCAAGGCTGTGGCATTCTGGGCG |
| | CTGTGACCTCTTCCAGAAGAAAGACTACATACGGACCTGCATCATGAACAATGGGGTT |
| | GGGTACCGGGGCACCATGGCCACGACCGTGGGTGGCCTGTCCTGCCAGGCTTGGAGCC |
| | ACAAGTTCCCGAATGATCACAAGTACATGCCCACGCTCCGGAATGGCCTGGAAGAGAA |
| | CTTCTGCCATAACCCTGATGGCGACCCCGGAGGTCCTTGGTGCCACACAACAGACCCT |
| | GCCGTGCGCTTCCAGAGCTGCGGCATCAAATCCTGCCGGGTGGCCGCGTGTGTCTGGT |
| | GCAATGGCGAGGAATACCGCGGCGCGGTAGACCGCACCGAGTCAGGGCGCGAGTGCCA |
| | GCGCTGGGATCTTCAGCACCCGCACCAGCACCCCTTCGAGCCGGGCAGGTTCCTCGAC |
| | CAAGGTCTGGACGACAACTATTGCCGGAATCCTGACGGCTCCGAGCGGCCATGGTGCT |
| | ACACTACGGATCCGCAGATCGAGCGAGAATTCTGTGACCTCCCCCGCTGCGGTTCCGA |
| | GGCACAGCCCCGCCAAGAGGCCACAAGTGTCAGCTGCTTCCGCGGGAAGGGTGAGGGC |
| | TACCGGGGCACAGCCAATACCACCACCGCGGGCGTACCTTGCCAGCGTTGGGACGCGC |
| | AAATCCCGCATCAGCACCGATTTACGCCAGAAAAATACGCGTGCAAGGACCTTCGGGA |
| | GAACTTCTGCCGGAACCTCGACGGCTCAGAGGCGCCCTGGTGCTTCACACTGCGGCCC |
| | GGCATGCGCGTGGGCTTTTGCTACCAGATCCGGCGTTGTACAGACGACGTGCGGCCCC |
| | AGGACTGCTACCACGGCGCGGGGGAGCAGTACCGCGGCACGGTCAGCAAGACCCGCAA |
| | GGGTGTCCAGTGCCAGCGCGCGTCCGCTGAGACGCCGCACAAGCCGCAGTTCACGTTT |
| | ACCTCCGAACCGCATGCACAACTGGAGGAGAACTTCTGCCAGACCCCAGATGGGGATA |
| | GCCATGGGCCCTGGTGCTACACGATGGACCCAAGGACCCCATTCGACTACTGTGCCCT |
| | GCGACGCTGCGCTGATGACCAGCCGCCATCAATCCTGGACCCCCCCGACCAGGTGCAG |
| | TTTGAGAAGTGTGGCAAGAGGGTGGATCGGCTGGATCAGCGTCGTTCCAAGCTGCGCG |
| | TGGCTGGGGGCCATCCGGGCAACTCACCCTGGACAGTCAGCTTGGGGAATCGGCAGGG |
| | CCAGCATTTCTGCGGGGGGTCTCTAGTGAAGGAGCAGTGGATACTGACTGCCCGGCAG |
| | TGCTTCTCCTCCCAGCATATGCCTCTCACGGGCTATGAGGTATGGTTGGGCACCCTGT |
| | TCCAGAACCCACAACATGGAGAGCCAGGCCTACAGCGGGTCCCAGTAGCCAAGATGCT |
| | GTGTGGGCCCTCAGGCTCCCAGCTTGTCCTGCTCAAGCTGGAGAGGTCTGTGACCCTG |
| | AACCAGCGTGTGGCCCTGATCTGCCTGCCGCCTGAATGATATGTGGTGCCTCCAGGGA |
| | CCAAGTGTGAGATTGCAGGCCGGGGTGAGACCAAAGGTAAGAGCATAGTGCACAGGAC |
| | TGCTGGTGGCCAGGAGGCCCAGCCC |
| | ORF Start: ATG at 76 ORF Stop: TGA at 1777 |
| | SEQ ID NO: 148 567 aa MW at 64065.2kD |
| NOV45d, | MTSRCSGAQSYLLHAVVPGPWQEDVADAEECAGRCGPLTDCWAFHYNVSSHGCQLLPW |
| CG54479-04 | |
| Protein | TQHSPHSRLWHSGRCDLFQKKDYIRTCIMNNGVGYRGTMATTVGGLSCQAWSHKFPND |
| Sequence | |
| | HKYMPTLRNGLEENFCHNPDGDPGGRWCHTTDPAVRFQSCGIKSCRVAACVWCNGEEY |
| | RGAVDRTESGRECQRWDLQHPHQHPFEPGRFLDQGLDDNYCRNPDGSERPWCYTTDPQ |
| | IEREFCDLPRCGSEAQPRQEATSVSCFRGKGEGYRGTANTTTAGVPCQRWDAQIPHQH |
| | RFTPEKYACKDLRENFCRNLDGSEAPWCFTLRPGMRVGFCYQIRRCTDDVRPQDCYHG |
| | AGEQYRGTVSKTRKGVQCQRASAETPHKPQFTFTSEPHAQLEENFCQTPDGDSHGPWC |
| | YTMDPRTPPDYCALRRCADDQPPSILDPPDQVQFEKCGKRVDRLDQRRSKLRVAGGHP |
| | GNSPWTVSLGNRQGQHFCGGSLVKEQWILTARQCFSSQHMPLTGYEVWLGTLFQNPQH |
| | GEPGLQRVRVAKMLCGPSGSQLVLLKLERSVTLNQRVALICLPPE |
| | SEQ ID NO: 149 1698 bp |
| NOV45e. | ATGACTTCTAGGTGCTCCGGGGCACAGAGCTACCTACAAGCGGTGGTGCCCGGGCCTT |
| CG54479-03 | |
| DNA | GGCAGGAGGATGTGGCAGATGCTGAAGAGTGTGCTGGTCGCTGTGGGCCCTTAATGGA |
| Sequence | |
| | CTGCGCGTTCCACTACAATGTGAGCAGCCATGGTTGCCAACTGCTGCCATGGACTCAA |
| | CACTCACCCCACACGAGGCTGCGGCATTCTGGGCGCTGTGACCTCTTCCAGGAGAAAG |
| | ACTACATACGGACCTGCATCATGAACAATGGGGTTGGGTACCGGGGCACCATGGCCAC |
| | GACCGTGGGTGGCCTGTCCTGCCAGGCTTGGAGCCACAAGTTCCCGAACGATCACCAG |
| | TACATGCCCACGCTCCGGAATGGCCTGGAAGAGAACTTCTGCCGTAACCCTGATGGCG |
| | ACCCCGGAGGTCCTTGGTGCCACACAACAGACCCTGCCGTGCGCTTCCAGAGCTGCGG |
| | CATCAAATCCTGCCGGGTGGCCGCGTGTGTCTGGTGCAATGGCGAGGAATACCGCGGC |
| | GCGGTAGACCGCACCGAGTCAGGGCGCGAGTGCCAGCGCTGGGATCTTCAGCACCCGC |
| | ACCAGCACCCCTTCGAGCCGGGCAAGTTCCTCGACCAAGGTCTGGACGACAACTATTG |
| | CCGGAATCCTGACGGCTCCGAGCGGCCATGGTGCTACACTACCGATCCGCAGATCGAG |
| | CGAGAATTCTGTGACCTCCCCCGCTGCGGTTCCGAGGCACAGCCCCGCCAAGAGGCCA |
| | CAAGTGTCAGCTGCTTCCGCGGGAAGGGTGAGGGCTACCGGGGCACAGCCAATACCAC |
| | CACCGCGGGCGTACCTTGCCAGCGTTGGGACGCGCAAATCCCGCATCAGCACCGATTT |
| | ACGCCAGAAAAATACGCGTGCAAGGACCTTCGGGAGAACTTCTGCCGGAACCCCGACG |
| | GCTCAGAGGCGCCCTGGTGCTTCACACTGCGGCCCGGCATGCGCGTGGGCTTTTGCTA |
| | CCAGATCCGGCGTTGTACAGACGACGTGCGGCCCCAGGACTGCTACCACGGCGCGGGG |
| | GAGCAGTACCGCGGCACGGTCAGCAAGACCCGCAAGGGTGTCCAGTGCCAGCGCGGGT |
| | CCGCTGAGACGCCGCACAAGCCGCAGTTCACGTTTACCTCCGAACCGCATGCACAACT |
| | GGAGGAGAACTTCTGCCAGGACCCAGATGGGGATAGCCATGGGCCCTGGTGCTACACG |
| | ATGGACCCAAGGACCCCATTCGACTACTGTGCCCTGCGACGCTGCGCTGATGACCAGC |
| | CGCCATCAATCCTGGACCCCCCCGACCAGGTGCAGTTTGAGAAGTGTGGCAAGAGGGT |
| | GGATCGGCTGGATCAGCGTTGTTCCAAGCTGCGCGTGGCTGGGGGCCATCCGGGCAAC |
| | TCACCCTGGACAGTCAGCTTGCGGAATAGGCAGGGCCAGCATTTCTGCGGGGGGTCTC |
| | TAGTGAAGGAGCAGTGGATACTGACTGCCCGGCAGTGCTTCTCCTCCAGCCATATGCC |
| | TCTCACGGGCTATGAGGTATGGTTGGGCACCCTGTTCCAGAACCCACAACATGGAGAG |
| | CCAGGCCTACAGCGGGTCCCAGTAGCCAAGATGCTGTGTGGGCCCTCAGGCTCTCAGC |
| | TTGTCCTGCTCAAGCTGGAGAGGTCTGTCACCCTGAACCAGCGTGTGGCCCTGATCTG |
| | CCTGCCGCCTGAATGA |
| | ORF Start: ATG at 1 ORF Stop: TGA at 1696 |
| | SEQ ID NO: 150 565 aa MW at 63751.8kD |
| NOV45e, | MTSRCSGAQSYLQAVVPGPWQEDVADAEECAGRCGPLMDCAFHYNVSSHGCQLLPWTQ |
| CG54479-05 | |
| Protein | HSPHTRLRHSGRCDLFQEKDYIRTCIMNNGVGYRGTMATTVGGLSCQAWSHKFPNDHQ |
| Sequence | |
| | YMPTLRNGLEENFCRNPDGDPGGPWCHTTDPAVRFQSCGIKSCRVAACVWCNGEEYRG |
| | AVDRTESGRECQRWDLQHPHQHPFEPGKFLDQGLDDNYCRNRDGSERPWCYTTDPQIE |
| | REFCDLRRCGSEAQPRQEATSVSCFRGKGEGYRGTANTTTAGVPCQRWDAQIPHQHRF |
| | TPEKYACKDLRENFCRNPDGSEAPWCFTLRPGMRVGFCYQIRRCTDDVRPQDCYHGAG |
| | EQYRGTVSKTRKGVQCQRGSAETPHKRQFTFTSEPHAQLEENFCQDPDGDSHGPWCYT |
| | MDRRTPFDYCALRRCADDQPPSILDPPDQVQPEKCGKRVDRLDQRCSKLRVAGGHPGN |
| | SPWTVSLRNRQGQHFCGGSLVKEQWILTARQCFSSSHMPLTGYEVWLGTLFQNPQHGE |
| | PGLQRVPVAKMLCGPSGSQLVLLKLERSVTLNQRVALICLPPE |
| | SEQ ID NO: 151 2066 bP |
| NOV45f, | ACAGGTTTCACAACTTCCCGGATGGGGCTGTGGTGGGTCACAGTGCAGCCTCCAGCCA |
| CG54479-06 | |
| DNA | GAAGGATGGGGTGGCTCCCACTCCTGCTGCTTCTGACTCAATGCTTAGGGGTCCCTGG |
| Sequence | |
| | GCAGCGCTCGCCATTGAATGACTTCCAAGTGCTCCGGGGCACAGAGCTACAGCACCTG |
| | CTACATGCGGTGGTGCCCGGGCCTTGGCAGGAGGATGTGGCAGATGCTGAAGAGTGTG |
| | CTGGTCGCTGTGGGCCCTTAATGGACTGCCGGGCCTTCCACTACAACGTGAGCAGCCA |
| | TGGTTGCCAACTGCTGCCATGGACTCAACACTCGCCCCACACGAGGCTGCGGCGTTCT |
| | GGGCGCTGTGACCTCTTCCAGAAGAAAGACTACGTACGGACCTGCATCATGAACAATG |
| | GGGTTGGGTACCGGGGCACCATGGCCACGACCGTGGGTGGCCTGCCCTGCCAGGCTTG |
| | GAGCCACAAGTTCCCGAATGATCACAAGTACACGCCCACTCTCCGGAATGGCCTGGAA |
| | GAGAACTTCTGCCGTAACCCTGATGGCGACCCCGGAGGTCCTTGGTGCTACACAACAG |
| | ACCCTGCTGTGCGCTTCCAGAGCTGCGGCATCAAATCCTGCCGGGAGGCCGCGTGTGT |
| | CTGGTGCAATGGCGAGGAATACCGCGGCGCGGTAGACCGCACGGAGTCAGGGCGCGAG |
| | TGCCAGCGCTGGGATCTTCAGCACCCGCACCAGCACCCCTTCGAGCCGGGCAAGTTCC |
| | TCGACCAAGGTCTGGACGACAACTATTGCCGGAATCCTGACGGCTCCGAGCGGCCATG |
| | GTGCTACACTACGGATCCGCAGATCGAGCGAGAGTTCTGTGACCTCCCCCGCTGCGGG |
| | TCCGAGGCACAGCCCCGCCAAGAGGCCACAACTGTCAGCTGCTTCCGCGGGAAGGGTG |
| | AGGGCTACCGGGGCACAGCCAATACCACCACTGCGGGCGTACCTTGCCAGCGTTGGGA |
| | CGCGCAAATCCCTCATCAGCACCGATTTACGCCAGAAAAATACGCGTGCAAAGACCTT |
| | CGGGAGAACTTCTGCCGGAACCCCGACGGCTCAGAGGCGCCCTGGTGCTTCACACTGC |
| | GGCCCGGCATGCGCGCGGCCTTTTGCTACCAGATCCGGCGTTGTACAGACGACGTGCG |
| | GCCCCAGGACTGCTACCACGGCGCAGGGGAGCAGTACCGCGGCACGGTCAGCAAGACC |
| | CGCAAGGGTGTCCAGTGCCAGCGCTGGTCCGCTGAGACGCCGCACAAGCCGCAGTTCA |
| | CGTTTACCTCCGAACCGCATGCACAACTGGAGGAGAACTTCTGCCGGAACCCAGATGG |
| | GGATAGCCATGGGCCCTGGTGCTACACGATGGACCCAAGGACCCCATTCGACTACTGT |
| | GCCCTGCGACGCTGCGCTGATGACCAGCCGCCATCAATCCTGGACCCCCCAGACCAGG |
| | TGCAGTTTGAGAAGTGTGGCAAGAGGGTGGATCGGCTGGATCAGCGGCGTTCCAAGCT |
| | GCGCGTGGTTGGGGGCCATCCGGGCAACTCACCCTGGACAGTCAGCTTGCGGAATCGG |
| | CAGGGCCAGCATTTCTGCGGGGGGTCTCTAGTGAAGGAGCAGTGGATACTGACTGCCC |
| | GGCAGTGCTTCTCCTCCTGCCATATGCCTCTCACGGGCTATGAGGTATGGTTGGGCAC |
| | CCTGTTCCAGAACCCACAGCATGGAGAGCCAAGCCTACAGCGGGTCCCAGTAGCCAAG |
| | ATGGTGTGTGGGCCCTCAGGCTCCCAGCTTGTCCTGCTCAAGCTGGAGAGATCTGTGA |
| | CCCTGAACCAGCGTGTGGCCCTGATCTGCCTGCCCCCTGAATGGTATGTGGTGCCTCC |
| | AGGGACCAAGTGTGAGGGTGACTACGGGGGCCCACTTGCCTGCTTTACCCACAACTGC |
| | TGGGTCCTGGAAGGAATTATAATCCCCAACCGAGTATGCGCAAGGTCCCGCTGGCCAG |
| | CTGTCTTCACGCGTGTCTCTGTGTTTGTGGACTGGATTCACAAGGTCATGAGACTGGG |
| | TTAGGCCCAGCCTTGATGCCATATGCCTTGGGGAGG |
| | ORF Start: ATG at 22 ORF Stop: TAG at 2032 |
| | SEQ ID NO: 152 670 aa MW at 76160.6kD |
| NOV45f, | MGLWWVTVQPPARRMGWLPLLLLLTQCLGVPGQRSPLNDFQVLRGTELQHLLHAVVPG |
| CG54479-06 | |
| Protein | PWQEDVADAEECAGRCGPLMDCRAFHYNVSSHGCQLLPWTQHSPHTRLRRSGRCDLFQ |
| Sequence | |
| | KKDYVRTCIMNNGVGYRGTMATTVGGLPCQAWSHKFPNDHKYTPTLRNGLEENPCRNP |
| | DGDPGGPWCYTTDPAVRFQSCGIKSCREAACVVCNGEEYRGAVDRTESGRECQRWDLQ |
| | HPHQHPFEPGKFLDQGLDDNYCRNPDGSERPWCYTTDPQIEREFCDLPRCGSEAQPRQ |
| | EATTVSCFRGKGEGYRGTANTTTAGVPCQRWDAQIPHQHRFTPEKYACKDLRENFCRN |
| | PDGSEAPWCFTLRPGMRAAFCYQIRRCTDDVRPQDCYHGAGEQYRGTVSKTRKGVQCQ |
| | RWSAETPHKPQETFTSEPHAQLEENFCRNPDGDSHGPWCYTMDPRTPFDYCALRRCAD |
| | DQPPSTLDPPDQVQFEKCGKRVDRLDQRRSKLRVVGGHPGNSPWTVSLRNRQGQHFCG |
| | GSLVKEQWILTARQCFSSCHMPLTGYEVWLGTLFQNPQHGEPSLQRVPVAKMVCGPSG |
| | SQLVLLKLERSVTLNQRVALICLPPEWYVVPPGTKCEGDYGGPLACFTHNCWVLEGII |
| | IPNRVCARSRWPAVFTRVSVFVDWIHKVMRLG |

Sequence comparison of the above protein sequences yields the following sequence relationships shown in Table 45B.

TABLE 45B
Comparison of NOV45a against NOV45b through NOV45f

| | | Identities/ |
|---|---|---|
| Protein | NOV45a Residues/ | Similarities for |
| Sequence | Match Residues | the Matched Region |
| NOV45b | 37 . . . 592 | 540/558 (96%) |
| | 12 . . . 568 | 545/558 (96%) |
| NOV45c | 110 . . . 448 | 319/339 (94%) |
| | 1 . . . 333 | 326/339 (96%) |
| NOV45d | 37 . . . 592 | 536/556 (96%) |
| | 12 . . . 567 | 543/556 (97%) |
| NOV45e | 35 . . . 592 | 547/558 (98%) |
| | 9 . . . 565 | 551/558 (98%) |
| NOV45f | 1 . . . 712 | 605/712 (84%) |
| | 15 . . . 670 | 619/712 (85%) |

Further analysis of the NOV45a protein yielded the following, properties shown in Table 45C.

TABLE 45C
Protein Sequence Properties NOV45a

| | |
|---|---|
| PSort | 0.4202 probability located in lysosome (lumen); 0.3700 |
| analysis: | probability located in outside; 0.1270 probability located |
| | in microbody (peroxisome); 0.1000 probability located in |
| | endoplasmic reticulum (membrane) |
| SignalP | Cleavage site between residues 19 and 20 |
| analysis: | |

A search of the NOV45a protein against the Geneseq database a proprietary database that contains sequences published in patents and patent publication, yielded several homologous proteins shown in Table 45D.

TABLE 45D
Geneseq Results for NOV45a

| | | NOV45a | | |
|---|---|---|---|---|
| | Protein/Organism/ | Residues/ | Identities/ | |
| Geneseq | Length [Patent #, | Match | Similarities for | Expect |
| Identifier | Date] | Residues | the Matched Region | Value |
| AAE16349 | Human MSP precursor-like protein, | 1 . . . 712 | 712/712 (100%) | 0.0 |
| | POLY13-Homo sapiens, 712 aa. | 1 . . . 712 | 712/712 (100%) | |
| | [WO200185767-A2, 15 Nov. 2001] | | | |
| AAW14270 | Human growth factor L5/3-Homo | 1 . . . 712 | 683/712 (95%) | 0.0 |
| | sapiens, 711 aa. [U.S. Pat. No. 5606029-A, | 1 . . . 711 | 693/712 (96%) | |
| | 25 Feb. 1997] | | | |
| AAR66602 | Human L5/3 tumour suppressor | 1 . . . 712 | 683/712 (95%) | 0.0 |
| | protein-Homo sapiens, 711 aa. | 1 . . . 711 | 693/712 (96%) | |
| | [U.S. Pat. No. 5315000-A, 24 May 1994] | | | |
| AAY31157 | Human macrophage stimulating | 1 . . . 712 | 682/712 (95%) | 0.0 |
| | protein-Homo sapiens, 711 aa. | 1 . . . 711 | 692/712 (96%) | |
| | [U.S. Pat. No. 5948892-A, 7 Sep. 1999] | | | |
| AAW82789 | Human MSP protein-Homo sapiens, | 1 . . . 712 | 682/712 (95%) | 0.0 |
| | 711 aa. [WO9855141-A1, | 1 . . . 711 | 692/712 (96%) | |
| | 10 Dec. 1998] | | | |

In a BLAST search of public sequence datbases, the NOV45a protein was found to have homology to the proteins shown in the BLASTP data in Table 45E.

TABLE 45E
Public BLASTP Results for NOV45a

| | | NOV45a | | |
|---|---|---|---|---|
| Protein | | Residues/ | Identities/ | |
| Accession | | Match | Similarities for | Expect |
| Number | Protein/Organism/Length | Residues | the Matched Portion | Value |
| P26927 | Hepatocyte growth factor-like protein | 1 . . . 712 | 682/712 (95%) | 0.0 |
| | precursor (Macrophage stimulatory | 1 . . . 711 | 692/712 (96%) | |
| | protein) (MSP) (Macrophage stimulating | | | |
| | protein)-Homo sapiens (Human), 711 aa. | | | |
| A40332 | macrophage-stimulating protein 1 | 1 . . . 711 | 555/720 (77%) | 0.0 |
| | precursor-mouse, 716 aa. | 1 . . . 715 | 621/720 (86%) | |
| P70521 | Macrophage stimulating protein precursor- | 1 . . . 711 | 556/720 (77%) | 0.0 |
| | Rattus norvegicus (Rat), 716 aa. | 1 . . . 715 | 616/720 (85%) | |
| P26928 | Hepatocyte growth factor-like protein | 1 . . . 711 | 554/720 (76%) | 0.0 |
| | precursor (Macrophage stimulatory | 1 . . . 715 | 620/720 (85%) | |
| | 716 aa. | | | |
| Q91XG8 | Hepatocyte growth factor-like-Mus | 1 . . . 711 | 552/720 (76%) | 0.0 |
| | musculus (Mouse), 716 aa. | 1 . . . 715 | 619/720 (85%) | |

PFam analysis predicts that the NOV45a protein contains the domains shown in the Table 45F.

TABLE 45F
Domain Analysis of NOV45a

| | NOV45a | Identities/Similarities | Expect |
|---|---|---|---|
| Pfam Domain | Match Region | for the Matched Region | Value |
| PAN | 18 . . . 106 | 23/110 (21%) | 3.6e−15 |
| | | 67/110 (61%) | |
| kringle | 110 . . . 186 | 41/85 (48%) | 1.3e−42 |
| | | 69/85 (81%) | |
| kringle | 191 . . . 268 | 48/85 (56%) | 1.7e−48 |
| | | 74/85 (87%) | |
| kringle | 283 . . . 361 | 44/85 (52%) | 1.9e−49 |
| | | 74/85 (87%) | |
| kringle | 370 . . . 448 | 42/85 (49%) | 4.3e−42 |
| | | 73/85 (86%) | |
| trypsin | 484 . . . 705 | 87/263 (33%) | 1e−45 |
| | | 160/263 (61%) | |

### Example 46

The NOV46 clone was analyzed, and the nucleotide and encoded polypeptide sequences are shown in Table 46A.

TABLE 46A
NOV46 Sequence Analysis

| | |
|---|---|
| | SEQ ID NO: 153 2412 bp |
| NOV46a, | ATGGGAAGCCAGTAACACTGTGGCCTACTATCTCTTCCGTGGTGCCATCTACATTTTT |
| CG56649-01 | |
| DNA | GGGACTCGGGAATTATGAGGTAGAGGTGGAGGCGGAGCCGGATGTCAGAGGTCCTGAA |
| Sequence | |
| | ATAGTCACCATGGGGGAAAATGATCCGCCTGCTGTTGAAGCCCCCTTCTCATTCCGAT |
| | CGCTTTTTGGCCTTGATGATTTGAAAATAAGTCCTCTTGCACCAGATGCAGATGCTGT |
| | TGCTGCACAGATCCTGTCACTGCTGCCATTGAAGTTTTTTCCAATCATCGTCATTGGG |
| | ATCATTGCATTGATATTAGCACTGGCCATTGGTCTGGGCATCCACTTCGACTGCTCAG |
| | GGAAGTACAGATGTCGCTCATCCTTTAAGTGTATCGAGCTGATAGCTCGATGTGACGG |
| | AGTCTCGGATTGCAAAGACGGGGAGGACGAGTACCGCTGTGTCCGGGTGGGTGGTCAG |
| | AATGCCGTGCTCCAGGTGTTCACAGCTGCTTCGTGGAAGACCATGTGCTCCGATGACT |
| | GGAAGGGTCACTACGCAAATGTTGCCTGTGCCCAACTGGGTTTCCCAAGCTATGTGAG |
| | TTCAGATAACCTCAGAGTGAGCTCGCTGGAGGGGCAGTTCCGGGAGGAGTTTGTGTCC |
| | ATCGATCACCTCTTGCCAGATGACAAGGTGACTGCATTACACCACTCAGTATATGTGA |
| | GGGAGGGATGTGCCTCTGGCCACGTGGTTACCTTGCAGTGCACAGCCTGTGGTCATAG |
| | AAGGGGCTACAGCTCACGCATCGTGGGTGGAAACATGTCCTTGCTCTCGCAGTGGCCC |
| | TGGCAGGCCAGCCTTCAGTTCCAGGGCTACCACCTGTGCGGGGGCTCTGTCATCACGC |
| | CCCTGTGGATCATCACTGCTGCACACTGTGTTTATGACTTGTACCTCCCCAAGTCATG |
| | GACCATCCAGGTGGGTCTAGTTTCCCTGTTGGACAATCCAGCCCCATCCCACTTGGTG |
| | GAGAAGATTGTCTACCACAGCAAGTACAAGCCAAAGAGGCTGCGCAATGACATCGCCC |
| | TTATGAAGCTGGCCGGGCCACTCACGTTCAATGAAATGATCCAGCCTGTGTGCCTGCC |
| | CAACTCTGAAGAGAACTTCCCCGATGGAAAAGTGTGCTGGACGTCAGGATGGGGGGCC |
| | ACAGAGGATGGAGGTGACCCCTCCCCTGTCCTGAACCACGCGGCCGTCCCTTTGATTT |
| | CCAACAAGATCTGCAACCACAGGGACGTGTACCGTGGCATCATCTCCCCCTCCATGCT |
| | CTGCGCGGGCTACCTGACGGGTGGCGTGGACAGCTGCCAGGGGGACAGCGGGGGGCCC |
| | CTGGTGTGTCAACAGAGGAGGCTGTGGAAGTTAGTGGGAGCGACCAGCTTTGGCATCG |
| | GCTGCGCAGAGGTGAACAAGCCTGGGGTGTACACCCGTGTCACCTCCTTCCTGGACTG |
| | GATCCACGAGCAGATGGAGAGAGACCTAAAAACCTGAAGAGGAAGGGGACAAGTAGCC |
| | ACCTGAGTTCCTGAGGTGATGAAGACAGCCCGATCCTCCCCTGGACTCCCGTGTAGGA |
| | ACCTGCACACGAGCAGACACCCTTGGAGCTCTGAGTTCCGGCACCAGTAGCAGGCCCG |
| | AAAGAGGCACCCTTCCATCTGATTCCAGCACAACCTTCAAGCTGCTTTTTGTTTTTTG |
| | TTTTTTTGAGGTGGAGTCTCGCTCTGTTGCCCAGGCTGGAGTGCAGTGGCGAAATCCC |
| | TGCTCACTGCAGCCTCCGCTTCCCTGGTTCAAGCGATTCTCTTGCCTCAGCTTCCCCA |
| | GTAGCTGGGACCACAGGTGCCCGCCACCACACCCAACTAATTTTTGTATTTTTAGTAG |
| | AGACAGGGTTTCACCATGTTGGCCAGGCTGCTCTCAAACCCCTGACCTCAAATGATGT |
| | GCCTGCTTCAGCCTCCCACAGTGCTGGGATTACAGGCATGGGCCACCACGCCTAGCCT |
| | CACGCTCCTTTCTGATCTTCACTAAGAACAAAAGAAGCAGCAACTTGCAAGGGCGGCC |
| | TTTCCCACTGGTCCATCTGGTTTTCTCTCCAGGGTCTTGCAAAATTCCTGACGAGATA |
| | AGCAGTTATGTGACCTCACGTGCAAAGCCACCAACAGCCACTCAGAAAAGACGCACCA |
| | GCCCAGAAGTGCAGAACTGCAGTCACTGCACGTTTTCATCTCTAGGGACCAGAACCAA |
| | ACCCACCCTTTCTACTTCCAAGACTTATTTTCACATGTGGGGAGGTTAATCTAGGAAT |
| | GACTCGTTTAAGGCCTATTTTCATGATTTCTTTGTAGCATTTGGTGCTTGACGTATTA |
| | TTGTCCTTTGATTCCAAATAATATGTTTCCTTCCCTCATTGTCTGGCGTGTCTGCGTG |
| | GACTGGTGACGTGAATCAAAATCATCCACTGAAA |
| | ORF Start: ATG at 126 ORF Stop: TGA at 1485 |
| | SEQ ID NO: 154 453 aa MW at 49333.0kD |
| NOV46a. | MGFNDPPAVEAPFSFRSLFGLDDLKISPVAPDADAVAAQILSLLPLKFFPIIVIGIIA |
| CG56649-01 | |
| Protein | LILALAIGLGIHFDCSGKYRCRSSFKCIELIARCDGVSDCKDGEDEYRCVRVGGQNAV |
| Sequence | |
| | LQVFTAASWKTMCSDDWKGHYANVACAQLGFPSYVSSDNLRVSSLEGQFREEFVSIDH |
| | LLPDDKVTALHHSVYVREGCASGHVVTLQCTACGHRRGYSSRIVGGNMSLLSQWPWQA |
| | SLQFQGYHLCGGSVITPLWIITAAHCVYDLYLPKSWTIQVGLVSLLDNPAPSHLVEKI |
| | VYHSKYKRKRLGNDIALMKLAGPLTFNEMIQPVCLPNSEENFRDGKVCWTSGWGATED |
| | GGDASRVLNHAAVPLISNKICNHRDVYGGIISPSMLCAGYLTGGVDSCQGDSGGRLVC |
| | QERRLWKLVGATSFGIGCAEVNKPGVYTRVTSPLDWIHEQMERDLKT |
| | SEQ ID NO: 155 1167 bp |
| NOV46b, | GGTACCATCCACTTCGACTGCTCAGGGAAGTACAGATGTCGCTCATCCTTTAAGTGTA |
| 169427553 | |
| DNA | TCGAGCTGATAGCTCGATGTGACGGAGTCTCGGATTGCAAAGACGGGGAGGACGAGTA |
| Sequence | |
| | CCGCTGTGTCCGGGTGAGTGGTCAGAATGCCGTGCTCCAGGTGTTCACAGCTGCTTCG |
| | TGGAAGACCATGTGCTCCGATGACTGGAAGGGTCACTACGCAAATGTTGCCTGTGCCC |
| | AACTGGGTTTCCCAAGCTATGTGAGTTCAGATAACCTCAGAGTGAGCTCGCTGGAGGG |
| | GCAGTTCCGGGAGGAGTTTGTGTCCATCGATCACCTCTTGCCAGATGACAAGGTGACT |
| | GCATTACACCACTCAGTATATGTGAGGGAGGGATGTGCCTCTGGCCACGTGGTTACCT |
| | TGCAGTGCACAGCCTGTGGTCATAGAAGGGGCTACAGCTCACGCATCGTGGGTGGAAA |
| | CATGTCCTTGCTCTCGCAGTGGCCCTGGCAGGCCAGCCTTCAGTTCCAGGGCTACCAC |
| | CTGTGCGGGGGCTCTGTCATCACGCCCCTGTGGATCATCACTGCTGCACACTGTGTTT |
| | ATGATTTGTACCTCCCCAAGTCATGGACCATCCAGGTGGGTCTAGTTTCCCTGTTGGA |
| | CAATCCAGCCCCATCCCACTTGGTGGAGAAGATTGTCTACCACAGCAAGTACAAGCCA |
| | AAGAGGCTGGGCAATGACATCGCCCTTATGAAGCTGGCCGGGCCACTCACGTTCAATG |
| | AAATGATCCAGCCTGTGTGCCTGCCCAACTCTGAAGAGAACTTCCCCGATGGAAAAGT |
| | GTGCTGGACGTCAGGATGGGGGGCCACAGAGGATGGAGGTGACGCCTCCCCTGTCCTG |
| | AACCACGCGGCCGTCCCTTTGATTTCCAACAAGATCTGCAACCACAGGGACGTGTACG |
| | GTGGCATCATCTCCCCCTCCATGCTCTGCGCGGGCTACCTGACGGGTGGCGTGGACAG |
| | CTGCCAGGGGGACAGCGGGGGGCCCCTGGTGTGTCAAGAGAGGAGGCTGTGGAAGTTA |
| | GTGGGAGCGACCAGCTTTGGCATCGGCTGCGCAGAGGTGAACAAGCCTGGGGTGTACA |
| | CCCGTGTCACCTCCTTCCTGGACTGGATCCACGAGCAGATGGAGAGAGACCTAAAAAC |
| | CCTCGAG |
| | ORF Start: at 1 ORF Stop: end of sequence |
| | SEQ ID NO: 156 389 aa MW at 42724.2kD |
| NOV46b, | GTIHFDCSGKYRCRSSFKCIELIARCDGVSDCKDGEDEYRCVRVSGQNAVLQVFTAAS |
| 169427553 | |
| Protein | WKTMCSDDWKGHYANVACAQLGFPSYVSSDNLRVSSLEGQFREEFVSIDHLLPDDKVT |
| Sequence | |
| | ALHHSVYVREGCASGHvVTLQCTACGHRRGYSSRIVGGNMSLLSQWPWQASLQFQGYH |
| | LCGGSVITPLWIITAAHCVYDLYLPKSWTIQVGLVSLLDNPAPSHLVEKIVYHSKYKP |
| | KRLGNDIALMKLAGPLTFNEMIQPVCLPNSEENFPDGKVCWTSGWGATEDGGDASPVL |
| | NHAAVPLISNKICNHRDVYGGIISPSMLCAGYLTGGVDSCQGDSGGPLVCQERRLWKL |
| | VGATSFGIGCAEVNKPGVYTRVTSFLDWIHEQMERDLKTLE |

Sequence comparison of the above protein sequences yields the following sequence relationships shown in Table 46B.

TABLE 46B
Comparison of NOV46a against NOV46b

| | NOV46a Residues/ | Identities/Similiarities |
|---|---|---|
| Protein Sequence | Match Residues | for the Matched Region |
| NOV46b | 69 . . . 453 | 384/385 (99%) |
| | 3 . . . 387 | 384/385 (99%) |

Further analysis of the NOV46a protein yielded the following properties shown in Table 46C.

TABLE 46C
Protein Sequence Properties NOV46a

| | |
|---|---|
| PSort | 0.6000 probability located in endoplasmic reticulum |
| analysis: | (membrane); 0.4413 probability located in microbody |
| | (peroxisome); 0.1000 probability located in mitochondrial |
| | inner membrane; 0.1000 probability located in plasma |
| | membrane |
| SignalP | Cleavage site between residues 69 and 70 |
| analysis: | |

A search of the NOV46a protein against the Geneseq database, a proprietary database that contains sequences published in patents and patent publication, yielded several homologous proteins shown in Table 46D.

TABLE 46D
Geneseq Results for NOV46a

| | Protein/ | NOV46a | Identities/ | |
|---|---|---|---|---|
| Geneseq | Organism/Length | Match | Matched | Expect |
| Identifier | [Patent #, Date] | Residues | Region | Value |
| AAE06935 | Human membrane- | 1 . . . 453 | 453/453 | 0.0 |
| | type serine | 1 . . . 453 | (100%) | |
| | protease (MTSP)6 - | | 453/453 | |
| | Homo sapiens, 453 aa. | | (100%) | |
| | [WO200157194-A2, | | | |
| | 09 AUG. 2001] | | | |
| AAU29055 | Human PRO polypeptide | 1 . . . 453 | 453/453 | 0.0 |
| | sequence #32 - Homo | 1 . . . 453 | (100%) | |
| | sapiens, 453 aa. | | 453/453 | |
| | [WO200168848-A2, | | (100%) | |
| | 20 SEP. 2001] | | | |
| AAB44250 | Human PRO382 | 1 . . . 453 | 452/453 | 0.0 |
| | (UNQ323) protein | 1 . . . 453 | (99%) | |
| | sequence SEQ ID | | 453/453 | |
| | NO: 69 - Homo | | (99%) | |
| | sapiens, 453 aa. | | | |
| | [WO200053756-A2, | | | |
| | 14 SEP. 2000] | | | |
| AAU82745 | Amino acid sequence of | 1 . . . 453 | 453/454 | 0.0 |
| | novel human protease | 1 . . . 454 | (99%) | |
| | #44 - Homo sapiens, | | 453/454 | |
| | 454 aa. | | (99%) | |
| | [WO200200860-A2, | | | |
| | 03 JAN. 2002] | | | |
| AAY41694 | Human PRO382 protein | 1 . . . 453 | 452/453 | 0.0 |
| | sequence - Homo | 1 . . . 452 | (99%) | |
| | sapiens, 452 aa. | | 452/453 | |
| | [WO9946281-A2, | | (99%) | |
| | 16 SEP. 1999] | | | |

In a BLAST search of public sequence datbases, the NOV46a protein was found to have homology to the proteins shown in the BLASTP data in Table 46E.

TABLE 46E
Public BLASTP Results for NOV46a

| | | | Identities/ | |
|---|---|---|---|---|
| | | | Similari- | |
| | | NOV46a | ties | |
| Protein | | Residues/ | for the | |
| Accession | Protein/ | Match | Matched | Expect |
| Number | Organism/Length | Residues | Portion | Value |
| CAC60382 | Sequence 11 from | 1 . . . 453 | 453/453 | 0.0 |
| | Patent WO0157194 - | 1 . . . 453 | (100%) | |
| | Homo sapiens | | 453/453 | |
| | (Human), 453 aa. | | (100%) | |
| P57727 | Transmembrane | 1 . . . 453 | 453/454 | 0.0 |
| | protease, | 1 . . . 454 | (99%) | |
| | serine 3 (EC 3.4.21.-) | | 453/454 | |
| | (Serine protease | | (99%) | |
| | TADG-12) (Tumor | | | |
| | associated | | | |
| | differentially- | | | |
| | expressed | | | |
| | gene-12 protein) - | | | |
| | Homo sapiens | | | |
| | (Human), 454 aa. | | | |
| Q8VDE0 | TMPRSS3 protein - | 1 . . . 453 | 402/453 | 0.0 |
| | Mus musculus | 1 . . . 453 | (88%) | |
| | (Mouse), 453 aa. | | 427/453 | |
| | | | (93%) | |
| Q8WY52 | Potential serine | 1 . . . 324 | 316/324 | 0.0 |
| | protease TMPRSS3 - | 1 . . . 324 | (97%) | |
| | Homo sapiens | | 317/324 | |
| | (Human), 344 aa. | | (97%) | |
| Q96T73 | Epitheliasin - | 52 . . . 450 | 188/411 | 4e−92 |
| | Homo sapiens | 89 . . . 491 | (45%) | |
| | (Human), 492 aa. | | 242/411 | |
| | | | (58%) | |

PFam analysis predicts that the NOV46a protein contains the domains shown in the Table 46F.

TABLE 46F
Domain Analysis of NOV46a

| | NOV46a | Identities/Similarities | Expect |
|---|---|---|---|
| Pfam Domain | Match Region | for the Matched Region | Value |
| Idl_recept_a | 71 . . . 109 | 15/43 (35%) | 0.00092 |
| | | 29/43 (67%) | |
| SRCR | 110 . . . 205 | 22/117 (19%) | 0.038 |
| | | 63/117 (54%) | |
| trypsin | 217 . . . 443 | 107/261 (41%) | 3.2e−92 |
| | | 179/261 (69%) | |

### Example 47

The NOV47 clone was analyzed, and the nucleotide and encoded polypeptide sequences are shown in Table 47A.

TABLE 47A
NOV47 Sequence Analysis

| | |
|---|---|
| | SEQ ID NO: 157 3149bp |
| NOV47a, | CTAAAGTTTTTTTCTTTGAATGACAGAACTACAGCATAATGCGTGGCTTCAACCTGCT |
| CG57209-01 | |
| DNA | CCTCTTCTGGGGATGTTGTGTTATGCACAGCTGGGAAGGGCACATAAGACCCACACGG |
| Sequence | |
| | AAACCAAACACAAAGGGTAATAACTGTAGAGACAGTACCTTGTGCCCAGCTTATGCCA |
| | CCTGCACCAATACGGTGGACAGTTACTATTGCACTTGCAAACAAGGCTTCCTGTCCAG |
| | CAATGGGCAAAATCACTTCAAGGATCCAGGAGTGCGATGCPAAGATATTGATGAATGT |
| | TCTCAAAGCCCCCAGCCCTGTGGTCCTAACTCATCCTGCAAAAACCTGTCAGGGAGGT |
| | ACAAGTGCAGCTGTTTAGATGGTTTCTCTTCTCCCACTGGAAATGACTGGGTCCCAGG |
| | AAAGCCGGGCAATTTCTCCTGTACTGATATCAATGAGTGCCTCACCAGCAGGGTCTGC |
| | CCTGAGCATTCTGACTGTGTCAACTCCATGGGAAGCTACAGTTGCAGCTGTCAAGTTG |
| | GATTCATCTCTAGAAACTCCACCTGTGAAGACGTGAATGAATGTGCAGATCCAAGAGC |
| | TTGCCCAGAGCATGCAACTTGTAATAACACTGTTGGAAACTACTCTTGTTTCTGCAAC |
| | CCAGGATTTGAATCCAGCAGTGGCCACTTGAGTTGCCAGGGTCTCAAAGCATCGTGTG |
| | AAGATATTGATGAATGCACTGAAATGTGCCCCATCAATTCAACATGCACCAACACTCC |
| | TGGGAGCTACTTTTGCACCTGCCACCCTGGCTTTGCACCAAGCAGTGGACAGTTGAAT |
| | TTCACAGACCAAGGAGTGGAATGTAGAGATATTGATGAGTGCCGCCAAGATCCATCAA |
| | CCTGTGGTCCTAATTCTATCTGCACCAATGCCCTGGGCTCCTACAGCTGTGGCTGCAT |
| | TGTAGGCTTTCATCCCAATCCAGAAGGCTCCCAGAAAGATGGCAACTTCAGCTGCCAA |
| | AGGGTTCTCTTCAAATGTAAGGAAGATGTGATACCCGATAATAAGCAGATCCAGCAAT |
| | GCCAAGAGGGAACCGCAGTGAAACCTGCATATGTCTCCTTTTGTGCACAAATAAATAA |
| | CATCTTCAGCGTTCTGGACAAAGTGTGTGAAAATAAAACGACCGTAGTTTCTCTGAAG |
| | AATACAACTGAGAGCTTTGTCCCTGTGCTTAAACAAATATCCATGTGGACTAAATTCA |
| | CCAAGGAAGAGACGTCCTCCCTGGCCACAGTCTTCCTGGAGAGTGTGGAAAGCATGAC |
| | ACTGGCATCTTTTTGGAAACCCTCAGCAAATGTCACTCCGGCTGTTCGGGCGGAATAC |
| | TTAGACATTGAGAGCAAAGTTATCAACAAAGAATGCAGTGAAGAGAATGTGACGTTGG |
| | ACTTGGTAGCCAAGGGGGATAAGATGAAGATCGGGTGTTCCACAATTGAGGAATCTGA |
| | ATCCACAGAGACCACTGGTGTGGCTTTTGTCTCCTTTGTGGGCATGGAATCGGTTTTA |
| | AATGAGCGCTTCTTCCAAGACCACCAGGCTCCCTTGACCACCTCTGAGATCAAGCTGA |
| | AGATGAATTCTCGAGTCGTTGGGGGCATAATGACTGGAGAGAAGAAAGACGGCTTCTC |
| | AGATCCAATCATCTACACTCTGGAGAACGTTCAGCCAAAGCAGAAGTTTGAGAGGCCC |
| | ATCTGTGTTTCCTGGAGCACTGATGTGAAGGGTGGAAGATGGACATCCTTTGGCTGTG |
| | TGATCCTGGAAGCTTCTGAGACATATACCATCTGCAGCTGTAATCAGATGGCAAATCT |
| | TGCCGTTATCATGGCGTCTGGGGAGCTCACGATGGACTTTTCCTTGTACATCATTAGC |
| | CATGTAGGCATTATCATCTCCTTGGTGTGCCTCGTCTTGGCCATCGCCACCTTTCTGC |
| | TGTGTCGCTCCATCCGAAATCACAACACCTACCTCCACCTGCACCTCTGCGTGTGTCT |
| | CCTCTTGGCGAAGACTCTCTTCCTCGCCGGTATACACAAGACTGACAACAAGACGGGC |
| | TGCGCCATCATCGCGGGCTTCCTGCACTACCTTTTCCTTGCCTGCTTCTTCTGGATGC |
| | TGGTGGAGGCTGTGATACTGTTCTTGATGGTCAGAAACCTGAAGGTGGTGAATTACTT |
| | CAGCTCTCGCAACATCAAGATGCTGCACATCTGTGCCTTTGGTTATGGGCTGCCGATG |
| | CTGGTGGTGGTGATCTCTGCCAGTGTGCAGCCACAGGGCTATGGAATGCATAATCGCT |
| | GCTGGCTGAATACAGAGACAGGGTTCATCTGGAGTTTCTTGGGGCCAGTTTGCACAGT |
| | TATAGTGATCAACTCCCTTCTCCTGACCTGGACCTTGTGGATCCTGAGGCAGAGGCTT |
| | TCCAGTGTTAATGCCGAAGTCTCAACGCTAAAAGACACCAGGTTACTGACCTTCAAGG |
| | CCTTTGCCCAGCTCTTCATCCTGGGCTGCTCCTGGGTGCTGGGCATTTTTCAGATTGG |
| | ACCTGTGGCAGGTGTCATGGCTTACCTGTTCACCATCATCAACAGCCTGCAGGGGGCC |
| | TTCATCTTCCTCATCCACTGTCTGCTCAACGGCCAGGTACGAGAAGAATACAAGAGGT |
| | GGATCACTGGGAAGACGAAGCCCAGCTCCCAGTCCCAGACCTCAAGGATCTTGCTGTC |
| | CTCCATGCCATCCGCTTCCAAGACGGGTTAAAGCCTTTCTTGCTTTCAAATATGCTAT |
| | GGAGCCACAGTTGAGGACAGTAGTTTCCTGCAGGAGCCTACCCTGAAATCTCTTCTCA |
| | GCTTAACATGGAAATGAGGATCCCACCAGCCCCAGAACCCTCTGGGGAAGAATGTTGG |
| | GGGCCGTCTTCCTGTGGTTGTATGCACTGATGAGAAATCAGACGTTTCTGCTCCAAAC |
| | GACCATTTTATCTTCGTGCTCTGCAACTTCTTCAATTCCAGAGTTTCTGAGAACAGAC |
| | CCAAATTCAATGGCATGACCAAGAACACCTGGCTACCATTTTGTTTTCTCCTGCCCTT |
| | GTTGGTGCATGGTTCTAAGCGTGCCCCTCCAGCGCCTATCATACGCCTGACACAGAGA |
| | ACCTCTCAATAAATGATTTGTCGCCTGTCTGACTGATTTACCCTAAAAAAAAAAAAAA |
| | AAAAAAAAAAAAAAAAA |
| | ORF Start: ATG at 39 ORF Stop: TAA at 2697 |
| | SEQ ID NO: 158 886 aa MW at 97679.1kD |
| NOV47a. | MRGFNLLLFWGCCVMHSWEGHIRPTRKPNTKGNNCRDSTLCRAYATCTNTVDSYYCTC |
| CG57209-01 | |
| Protein | KQGFLSSNGQNHFKDPGVRCKDIDECSQSRQPCGPNSSCKNLSGRYKCSCLDGFSSPT |
| Sequence | |
| | GNDWVPGKPGNFSCTDINECLTSRVCPEHSDCVNSMGSYSCSCQVGFISRNSTCEDVN |
| | ECADRRACPEHATCNNTVGNYSCFCNPGFESSSGHLSCQGLKASCEDIDECTEMCPIN |
| | STCTNTPGSYFCTCHPGPAPSSGQLNFTDQGVECRDIDECRQDPSTCGPNSICTNALG |
| | SYSCGCIVGFHPNPEGSQKDGNFSCQRVLFKCKEDVIPDNKQIQQCQEGTAVKPAYVS |
| | FCAQINNIFSVLDKVCENKTTVVSLKNTTESPVPVLKQISMWTKPTKEETSSLATVFL |
| | ESVESMTLASFWKPSANVTPAVRAEYLDIESKVINKECSEENVTLDLVAKGDKMKIGC |
| | STIEESESTETTGVAFVSFVGMESVLNERFFQDHQAPLTTSEIKLKMNSRVVGGIMTG |
| | EKKDGFSDPIIYTLENVQPKQKFFRPICVSWSTDVKGGRWTSFGCVILEASETYTTCS |
| | CNQMANLAVIMASGELTMDFSLYIISHVGIIISLVCLVLAIATFLLCRSIRNHNTYLH |
| | LHLCVCLLLAKTLFLAGIHKTDNKTGCAIIAGFLHYLFLACPFWMLVEAVILFLMVRN |
| | LKVVNYFSSRNIKMLHICAFGYGLPMLVVVISASVQPQGYGMHNRCWLNTETGFIWSF |
| | LGPVCTVIVINSLLLTWTLWILRQRLSSVNAEVSTLKDTRLLTFKAFAQLFILGCSWV |
| | LGIFQIGPVAGVMAYLFTIINSLQGAPIFLIHCLLNGQVREEYKRWITGKTKPSSQSQ |
| | TSRILLSSMPSASKTG |
| | SEQ ID NO: 159 12851 bp |
| NOV47b. | GCTCCTCTTCTGGGGTGTTGTGTTATGCACAGCTGGGAAGGGCACATAAGACCCACAC |
| CG57209-04 | |
| DNA | GGAAACCAAACACAAAGGGTAATAACTGTAGAGACAGTACCTTGTGCCCAGCTTATGC |
| Sequence | |
| | CACCTGCACCAATACAGTGGACAGTTACTATTGCGCTTGCAAACAAGGCTTCCTGTCC |
| | AGCAATGGGCAAAATCACTTCAAGGATCCAGGAGTGCGATGCAAAGATATTGATGAAT |
| | GTTCTCAAAGCCCCCAGCCCTGTGGTCCTAACTCATCCTGCAAAAACCTGTCAGGGAG |
| | GTACAAGTGCAGCTGTTTAGATGGTTTCTCTTCTCCCACTGGAAATGACTGGGTCCCA |
| | GGAAAGCCGGGCAATTTCTCCTGTACTGATATCAATGAGTGCCTCACCAGCAGCGTCT |
| | GCCCTGAGCATTCTGACTGTGTCAACTCCATGGGAAGCTACAGTTGTAGCTGTCAAGT |
| | TGGATTCATCTCTAGAAACTCCACCTGTGAAGACGTGGATGAATGTGCAGATCCAAGA |
| | GCTTGCCCAGAGCATGCAACTTGTAATAACACTGTTGGAAACTACTCTTGTTTCTGCA |
| | ACCCAGGATTTGAATCCAGCAGTGGCCACTTGAGTTTCCAGGGTCTCAAAGCATCGTG |
| | TGAAGATATTGATGAATGCACTGAAATGTGCCCCATCAATTCAACATGCACCAACACT |
| | CCTGGGAGCTACTTTTGCACCTGCCACCCTGGCTTTGCACCAAGCAATGGACAGTTGA |
| | ATTTCACAGACCAAGGACTGGAATGTAGAGATATTGATGAGTGCCGCCAAGATCCATC |
| | AACCTGTGGTCCTAATTCTATCTGCACCAATGCCCTGGGCTCCTGCAGCTGTGGCTGC |
| | ATTGCAGGCTTTCATCCCAATCCAGAAGGCTCCCAGAAAGATGGCAACTTCAGCTGCC |
| | AAAGGGTTCTCTTCAAATGTAAGGAAGATGTGATACCCGATAATAAGCAGATCCAGCA |
| | ATGCCAAGAGGGkACCGCAGTGAAACCTGCATATGTCTCCTTTTGTGCACAAATAAAT |
| | AACATCTTCAGCGTTCTGGACAAAGTGTGTGAAAATAAAACGACCGTAGTTTCTCTGA |
| | AGAATACAACTGAGAGCTTTGTCCCTGTGCTTAAACAAATATCCACGTGGACTAAATT |
| | CACCAAGGAAGAGACGTCCTCCCTGGCCACAGTCTTCCTGGAGAGTGTGGAkAGCATG |
| | ACACTGGCATCTTTTTGGAAACCCTCAGCAAATGTCACTCCGGCTGTTCGGACGGAAT |
| | ACTTAGACATTGAGAGCAAAGTTATCAACAAAGAATGCAGTGAAGAGAATGTGACGTT |
| | GGACTTGGTAGCCAAGGGGGATAAGATGAAGATCGGGTGTTCCACAATTGAGGAATCT |
| | GAATCCACAGAGACCACTGGTGTGGCTTTTGTCTCCTTTGTGGGCATGGAATCGGTTT |
| | TAAATGAGCGCTTCTTCCAAGACCACCAGGCTCCCTTGACCACCTCTGAGATCAAGCT |
| | GAAGATGAATTCTCGAGTCGTTGGGGGCATAATGACTGGAGAGAAGAAAGACGGCTTC |
| | TCAGATCCAATTATCTACACTCTGGAGAACGTTCAGCCAAAGCAGAAGTTTGAGAGGC |
| | CCATCTGTGTTTCCTGGAGCACTGATGTGAAGGGTGGAAGATGGACATCCTTTGGCTG |
| | TGTGATCCTGGAAGCTTCTGAGACATATACCATCTGCAGCTGTAATCAGATGGCAAAT |
| | CTTGCCGTTATCATGGCGTCTGGGGAGCTCACGATGGGCTGCGCCATCATCGCGGGCT |
| | TCCTGCACTACCTTTTCCTTGCCTGCTTCTTCTGGATGCTGGTGGAGGCTGTGATACT |
| | GTTCTTGATGGTCAGAAACCTGAAGGTGGTGAATTACTTCAGCTCTCGCAACATCAAG |
| | ATGCTGCACATCTGTGCCTTTGGTTATGGGCTGCCGATGCTGGTGGTGGTGATCTCTG |
| | CCAGTGTGCAGCCACAGGGCTATGGAATGCATAATCGCTGCTGGCTGAATACAGAGAC |
| | AGGGTTCATCTGGAGTTTCTTGGGGCCAGTTTGCACAGTTATAGTGATCAACTCCCTT |
| | CTCCTGACCTGGACCTTGTGGATCCTGAGGCAGAGGCTTTCCAGTGTTAATGCCGAAG |
| | TCTCAACGCTAAAAGACACCAGGTTACTGACCTTCAAGGCCTTTGCCCAGCTCTTCAT |
| | CCTGGGCTGCTCCTGGGTGCTGGGCATTTTTCAGATTGGACCTGTGGCAGGTGTCATG |
| | GCTTACCTGTTCACCATCATCAACAGCCTGCACGGGGCCTTCATCTTCCTCATCCACT |
| | GTCTGCTCAACGGCCAGGTACGAGAAGAATACAAGAGGTGGATCACTGGGAAGACGAA |
| | GCCCAGCTCCCAGTCCCAGACCTCAAGGATCTTGCTGTCCTCCATGCCATCCGCTTCC |
| | AAGACGGGTTAAAGTCCTTTCTTGCTTTCAAATATGCTATGGAGCCACAGTTGAGGAC |
| | AGTAGTTTCCTGCAGGAGCCTACCCTGAAATCTCTTCTCAGCTTAACATGGAAATGAG |
| | GATCCCACCAGCCCCAGAACCCTCTGGGGAAGAATGTTGGGGGCCGTCTTCCTGTGGT |
| | TGTATGCACTGATGAGAAATCAGGCGTTTCTGCTCCAAACGACCATTTTATCTTCGTG |
| | CTCTGCAACTTCTTCAATTCCAGAGTTTCTGAGAACAGACCCAAATTCAATGGCATGA |
| | CCAAGAACACCTGGCTACCATTTTGTTTTCTCCTGCCCTTGTTGGTGCATGGTTCTAA |
| | GCGTGCCCCTCCAGCGCCTATCATACGCCTGACACAGAGAACCTCTCAATAAATGATT |
| | TGTCGCCTG |
| | ORF Start: at 13 ORF Stop: TAA at 2446 |
| | SEQ ID NO: 160 811 aa MW at 89011.6kD |
| NOV47b, | GCCVMHSWEGHIRPTRKPNTKGNNCRDSTLCPAYATCTNTVDSYYCACKQGFLSSNGQ |
| CG57209-04 | |
| Protein | NHFKDPGVRCKDIDECSQSPQPCGPNSSCKNLSGRYKCSCLDGFSSPTGNDWVPGKPG |
| Sequence | |
| | NFSCTDTNECLTSSVCPEHSDCVNSMGSYSCSCQVGFTSRNSTCEDVDECADPRACPE |
| | HATCNNTVGNYSCFCNPGFESSSGHLSFQGLKASCEDIDECTEMCPINSTCTNTPGSY |
| | FCTCHPGFAPSNGQLNFTDQGVECRDIDECRQDPSTCGPNSICTNALGSCSCGCIAGF |
| | HPNPEGSQKDGNFSCQRVLFKCKEDVIPDNKQIQQCQEGTAVKPAYVSFCAQINNIFS |
| | VLDKVCENKTTVVSLKNTTESFVPVLKQISTWTKPTKEETSSLATVFLESVESMTLAS |
| | FWKPSANVTPAVRTEYLDIESKVINKECSEENVTLDLVAKGDKMKIGCSTTEESESTE |
| | TTGVAFVSFVGMESVLNERFFQDHQAPLTTSEIKLKMNSRVVGGIMTGEKKDGFSDPT |
| | IYTLENVQPKQKFERPICVSWSTDVKGGRWTSFGCVILEASETYTTCSCNQMANLAVI |
| | MASGELTMGCAIIAGFLHYLFLACFFWMLVEAVILFLMVRNLKVVNYFSSRNIKMLHI |
| | CAFGYGLPMLVVVTSASVQPQGYGMHNRCWLNTETGFIWSFLGPVCTVIVINSLLLTW |
| | TLWILRQRLSSVNAEVSTLKDTRLLTFKAFAQLFILGCSWVLGIFQTGPVAGVMAYLF |
| | TIINSLQGAFIFLIHCLLNGQVREEYKRWTTGKTKPSSQSQTSRILLSSMPSASKTG |
| | SEQ ID NO: 161 1764 bp |
| NOV47c, | AGATCTTGGGAAGGGCACATAAGACCCACACGGAAACCAAACACAAAGGGTAATAACT |
| 165275217 | |
| DNA | GTAGAGACAGTACCTTGTGCCCAGCTTATGCCACCTGCACCAATACAGTGGACAGTTA |
| Sequence | |
| | CTATTGCACTTGCAAACAAGGCTTCCTGTCCAGCAATGGGCAAAATCACTTCAAGGAT |
| | CCAGGAGTGCGATGCAAAGATATTGATGAATGTTCTCAAAGCCCCCAGCCCTGTGGTC |
| | CTAACTCATCCTGCAAAAACCTGTCAGGGAGGTACAAGTGCAGCTGTTTAGATGGTTT |
| | CTCTTCTCCCACTGGAAATGACTGGGTCCCAGGAAAGCCGGGCAATTTCTCCTGTACT |
| | GATATCAATGAGTGCCTCACCAGCAGGGTCTGCCCTGAGCATTCTGACTGTGTCAACT |
| | CCATGGGAAGCTACAGTTGCAGCTGTCAAGTTGGATTCATCTCTAGAAACTCCACCTG |
| | TGGAGACGTGAATGAATGTGCAGATCCAAGAGCTTGCCCAGAGCATGCAACTTGTAAT |
| | AACACTGTTGGAAACTACTCTTGTTTCTGCAACCCAGGATTTGAATCCAGCAGTGGCC |
| | ACTTGAGTTTCCAGGGTCTCAAAGCATCGTGTGAAGATATTGATGAATGCACTGAAAT |
| | GTGCCCCATCAATTCAACATGCACCAACACTCCTGGGAGCTACTTTTGCACCTGCCAC |
| | CCTGGCTTTGCACCAAGCAATGGACAGTTGAATTTCACAGACCAAGGAGTGGAATGTA |
| | GAGATATTGATGAGTGCCGCCAAGATCCATCAAACCTGTGGTCCTATTCTATCTGCAC |
| | CAATGCCCTGGGCTCCTACAGCTGTGGCTGCATTGTAGGCTTTCATCCCAATCCAGAA |
| | GGCTCCCAGAAAGATGGCAACTTCAGCTGTCAAAGGGTTCTCTTCAAATGTAAGGAAG |
| | ATGTGATACCCGATAATAAGCAGATCCAGCAATGCCAAGAGGGAACCGCAGTGAAACC |
| | TGCATATGTCTCCTTTTGTGCACAAATAAATAACATCTTCAGCGTTCTGGACAAAGTG |
| | TGTGAAAATAAAACGACCGTAGTTTCTCTGAAGAATACAACTGAGAGCTTTGTCCCTG |
| | TGCTTAAACAAATATCCACGTGGACTAAATTCACCAAGGAAGAGACGTCCTCCCTGGC |
| | CACAGTCTTCCTGGAGAGTGTGGAAAGCATGACACTGGCATCTTTTTGGAAACCCTCA |
| | GCAAATGTCACTCCGGCTGTTCGGACGGAATACTTAGACATTGAGAGCAAAGTTATCA |
| | ACAAAGAATGCAGTGAAGAGAATGTGACGTTGGACTTGGTAGCCAAGGGGGATAAGAT |
| | GAAGATCGGGTGTTCCACAATTGAGGAATCTGAATCCACAGAGACCACTGGTGTGGCT |
| | TTTGTCTCCTTTGTGGGCATGGAATCGGTTTTAAATGAGCGCTTCTTCCAAGACCACC |
| | AGGCTCCCTTGACCACCTCTGAGATCAAGCTGAAGATGAATTCTCGAGTCGTTGGGGG |
| | CATAATGACTGGAGAGAAGAAAGACGGCTTCTCAGATCCAATCATCTACACTCTGGAG |
| | AACGTTCAGCCAAAGCAGAAGTTTGAGAGGCCCATCTGTGTTTCCTGGAGCACTGATG |
| | TGAAGGGTGGAAGATGGACATCCTTTGGCTGTGTGATCCTGGAAGCTTCTGAGACATA |
| | TACCATCTGCAGCTGTAATCAGATGGCAAATCTTGCCGTTATCATGGCGTCTGCGGAG |
| | CTCACGGTCGACAAGGGCGAATTT |
| | ORF Start: at 1 ORF Stop: end of sequence |
| | SEQ ID NO: 162 588 aa MW at 64167.2kD |
| NOV47c, | RSWEGHIRPTRKPNTKGNNCRDSTLCPAYATCTNTVDSYYCTCKQGFLSSNGQNHPKD |
| 165275217 | |
| Protein | PGVRCKDIDECSQSPQPCGPNSSCKNLSGRYKCSCLDGFSSPTGNDWVPGKPGNFSCT |
| Sequence | |
| | DINECLTSRVCPEHSDCVNSMGSYSCSCQVGFISRNSTCGDVNECADPRACPEHATCN |
| | NTVGNYSCPCNPGFESSSGHLSPQGLKASCEDIDECTEMCPINSTCTNTPGSYPCTCH |
| | PGFAPSNGQLNFTDQGVECRDIDECRQDPSTCGRNSICTNALGSYSCGCIVGFHPNPE |
| | GSQKDGNFSCQRVLFKCKEDVIPDNKQIQQCQEGTAVKPAYVSPCAQINNIFSVLDKV |
| | CENKTTVVSLKNTTESFVPVLKQISTWTKFTKEETSSLATVFLESVESMTLASFWKPS |
| | ANVTPAVRTEYLDIESKVINKECSEENVTLDLVAKGDKMKIGCSTIEESESTETTGVA |
| | FVSFVGMESVLNERFFQDHQAPLTTSEIKLKMNSRVVGGIMTGEKKDGFSDPIIYTLE |
| | NVQPKQKFERPICVSWSTDVKGGRWTSFGCVILEASETYTTCSCNQMANLAVIMASGE |
| | LTVDKGEF |

Sequence comparison of the above protein sequences yields the following sequence relationships shown in Table 47B.

TABLE 47B
Comparison of NOV47a against NOV47b and NOV47c

| | NOV47a Residues/ | Identities/Similarities |
|---|---|---|
| Protein Sequence | Match Residues | for the Matched Region |
| NOV47b | 11 . . . 886 | 783/876 (89%) |
| | 1 . . . 811 | 788/876 (89%) |
| NOV47c | 17 . . . 599 | 565/583 (96%) |
| | 2 . . . 584 | 567/583 (96%) |

Further analysis of the NOV47a protein yielded the following properties shown in Table 47C.

TABLE 47C
Protein Sequence Properties NOV47a

| | |
|---|---|
| PSort | 0.6850 probability located in endoplasmic reticulum |
| analysis: | (membrane); 0.6400 probability located in plasma membrane: |
| | 0.4600 probability located in Golgi body; 0.1000 probability |
| | located in endoplasmic reticulum (lumen) |
| SignalP | Cleavage site between residues 18 and 19 |
| analysis: | |

A search of the NOV47a protein against the Geneseq database, a proprietary database that contains sequences published in patents and patent publication, yielded several homologous proteins shown in Table 47D.

TABLE 47D
Geneseq Results for NOV47a

| | | | Identities/ | |
|---|---|---|---|---|
| | | | Similari- | |
| | | NOV47a | ties | |
| | Protein/ | Residues/ | for the | |
| Geneseq | Organism/Length | Match | Matched | Expect |
| Identifier | [Patent #, Date] | Residues | Region | Value |
| AAB71869 | Human EMR1 seven | 1 . . . 886 | 886/886 | 0.0 |
| | transmembrane | 1 . . . 886 | (100%) | |
| | domain - Homo | | 886/886 | |
| | sapiens, 886 aa. | | (100%) | |
| | [WO200109328-A1, | | | |
| | 08 FEB. 2001] | | | |
| AAB01249 | Human EMR1 | 1 . . . 886 | 880/886 | 0.0 |
| | hormone receptor - | 1 . . . 880 | (99%) | |
| | Homo sapiens, 880 aa. | | 880/886 | |
| | [WO200034473-A2, | | (99%) | |
| | 15 JUN. 2000] | | | |
| AAE17043 | Human CD 97 | 74 . . . 872 | 272/853 | e−122 |
| | protein - Homo | 16 . . . 817 | (31%) | |
| | sapiens, 835 aa. | | 422/853 | |
| | [WO200202602-A2, | | (48%) | |
| | 10 JAN. 2000] | | | |
| AAB15728 | Human CD97 protein - | 74 . . . 872 | 272/853 | e−122 |
| | Homo sapiens, 835 aa. | 16 . . . 817 | (31%) | |
| | [WO200052039-A2, | | 422/853 | |
| | 08 SEP. 2000] | | (48%) | |
| AAY41090 | Human CD97 protein - | 74 . . . 872 | 272/853 | e−122 |
| | Homo sapiens, | 16 . . . 817 | (31%) | |
| | 835 aa. | | 422/853 | |
| | [WO9945111-A1, | | (48%) | |
| | 10 SEP. 1999] | | | |

In a BLAST search of public sequence datbases, the NOV47a protein was found to have homology to the proteins shown in the BLASTP data in Table 47E.

TABLE 47E
Public BLASTP Results for NOV47a

| | | | Identities/ | |
|---|---|---|---|---|
| | | | Similari- | |
| | | NOV47a | ties | |
| Protein | | Residues/ | for the | |
| Accession | Protein/ | Match | Matched | Expect |
| Number | Organism/Length | Residues | Portion | Value |
| Q14246 | Cell surface | 1 . . . 886 | 886/886 | 0.0 |
| | glycoprotein EMR1 | 1 . . . 886 | (100%) | |
| | precursor (EMR1 | | 886/886 | |
| | hormone receptor) - | | (100%) | |
| | Homo sapiens | | | |
| | (Human), 886 aa. | | | |
| Q61549 | Cell surface | 1 . . . 886 | 606/937 | 0.0 |
| | glycoprotein EMR1 | 1 . . . 931 | (64%) | |
| | precursor (EMR1 | | 709/937 | |
| | hormone receptor) | | (74%) | |
| | (Cell surface | | | |
| | glycoprotein F4/80) - | | | |
| | Mus musculus | | | |
| | (Mouse), 931 aa. | | | |
| Q9BY15 | EGF-like | 229 . . . 871 | 245/644 | e−127 |
| | module-containing | 31 . . . 625 | (38%) | |
| | mucin-like | | 370/644 | |
| | receptor EMR3 - | | (57%) | |
| | Homo sapiens | | | |
| | (Human), 652 aa. | | | |
| O00718 | CD97 - Homo | 74 . . . 872 | 272/853 | e−121 |
| | sapiens (Human), | 16 . . . 817 | (31%) | |
| | 835 aa. | | 422/853 | |
| | | | (48%) | |
| P48960 | Leucocyte antigen | 74 . . . 872 | 270/853 | e−120 |
| | CD97 precursor - | 16 . . . 817 | (31%) | |
| | Homo sapiens | | 420/853 | |
| | (Human), 835 aa. | | (48%) | |

PFam analysis predicts that the NOV47a protein contains the domains shown in the Table 47F.

TABLE 47F
Domain Analysis of NOV47a

| | NOV47a | Identities/Similarities | Expect |
|---|---|---|---|
| Pfam Domain | Match Region | for the Matched Region | Value |
| EGF | 35 . . . 70 | 13/47 (28%) | 0.29 |
| | | 26/47 (55%) | |
| TILa | 34 . . . 89 | 16/58 (28%) | 0.42 |
| | | 36/58 (62%) | |
| EGF | 176 . . . 212 | 15/47 (32%) | 0.0038 |
| | | 25/47 (53%) | |
| EGF | 225 . . . 255 | 13/47 (28%) | 0.29 |
| | | 23/47 (49%) | |
| GPS | 546 . . . 596 | 19/54 (35%) | 1.5e−18 |
| | | 46/54 (85%) | |
| 7tm_2 | 599 . . . 851 | 96/276 (35%) | 9.2e−104 |
| | | 228/276 (83%) | |

### Example 48

The NOV48 clone was analyzed, and the nucleotide and encoded polypeptide sequences are shown in Table 48A.

TABLE 48A
NOV48 Sequence Analysis

| | |
|---|---|
| | SEQ ID NO: 163 4080 bp |
| NOV48a, | GAGTGGAGTTCTGGAGGAATGTTTACCAGACACAGAGCCCAGAGGGACAGCGCCCAGA |
| CG59325-01 | |
| DNA | GCCCAGATAGAGAGACACGGCCTCACTGGCTCAGCACCAGGGTCCCCTTCCCCCTCCT |
| Sequence | |
| | CAGCTCCCTCCCTGGCCCCTTTAAGGAAAGAGCTGATCCTCTCCTCTCTTGAGTTAACC |
| | CCTGATTGTCCAGGTGGCCCCTGGCTCTGGCCTGGTGGGCGGAGGCAAAGGGGGAGCC |
| | AGGGGCGGAGAAAGGGTTGCCCAAGTCTGGGAGTGAGGGAAGGAGGCAGGGGTGCTGA |
| | GAAGGCGGCTGCTGGGCAGAGCCGGTGGCAAGGGCCTCCCCTGCCGCTGTGCCAGGCA |
| | GGCAGTGCCAAATCCGGCGAGCCTGGAGCTGGGGGGAGGGCCGGGGACAGCCCGGCCC |
| | GCTGCCCCCTCCCCCGCTGGGAGCCCAGCAACTTCTGAGGAAAGTTTGGCACCCATGG |
| | CGTGGCGGTGCCCCAGGATGGGCAGGGTCCCGCTGGCCTGGTGCTTGGCGCTGTGCGG |
| | CTGGGCGTGCATGGCCCCCAGGGGCACGCAGGCTGAAGAAAGTCCCTTCGTGGGCAAC |
| | CCAGGGAATATCACAGGTGCCCGGGGACTCACGGGCACCCTTCGGTGTCAGCTCCAGG |
| | TTCAGGGAGAGCCCCCCGAGGTACATTGGCTTCGGGATGGACAGATCCTGGAGCTCGC |
| | GGACAGCACCCAGACCCAGGTGCCCCTGGGTGAGGATGAACAGGATGACTGGATAGTG |
| | GTCAGCCAGCTCAGAATCACCTCCCTGCAGCTTTCCGACACGGGACAGTACCAGTGTT |
| | TGGTGTTTCTGGGACATCAGACCTTCGTGTCCCAGCCTGGCTATGTTGGGCTGGAGGG |
| | CTTGCCTTACTTCCTGGAGGAGCCCGAAGACAGGACTGTGGCCGCCAACACCCCCTTC |
| | AACCTGAGCTCCCAAGCTCAGGGACCCCCAGAGCCCGTGGACCTACTCTGGCTCCAGG |
| | ATGCTGTCCCCCTGGCCACGGCTCCAGGTCACGGCCCCCAGCGCAGCCTGCATGTTCC |
| | AGGGCTGAACAAGACATCCTCTTTCTCCTGCGAAGCCCATAACGCCAAGGGGGTCACC |
| | ACATCCCGCACAGCCACCATCACAGTGCTCCCCCAGCAGCCCCGTAACCTCCACCTGG |
| | TCTCCCGCCAACCCACGGAGCTGGAGGTGGCTTGGACTCCAGGCCTGAGCGGCATCTA |
| | CCCCCTGACCCACTGCACCCTGCAGGCTGTGCTGTCAGACGATGGGATGGGCATCCAG |
| | GCGGGAGAACCAGACCCCCCAGAGGAGCCCCTCACCTCGCAAGCATCCGTGCCCCCCC |
| | ATCAGCTTCGGCTAGGCAGCCTCCATCCTCACCCCCCTTATCACATCCGCGTGGCATG |
| | CACCAGCAGCCAGGGCCCCTCATCCTGGACCCACTGGCTTCCTGTGGAGACGCCGGAG |
| | GGAGTGCCCCTGGGCCCCCCTGAGAACATTAGTGCTACGCGGAATGGGAGCCAGGCCT |
| | TCGTGCATTGGCAAGAGCCCCGGGCGCCCCTGCAGGGTACCCTGTTAGGGTACCGGCT |
| | GGCGTATCAAGGCCAGGACACCCCAGAGGTGCTAATGGACATAGGGCTAAGGCAAGAG |
| | GTGACCCTGGAGCTGCAGGGGGACGGGTCTGTGTCCAATCTGACAGTGTGTGTGGCAG |
| | CCTACACTGCTGCTGGGGATGGACCCTGGAGCCTCCCAGTACCCCTGGAGGCCTGGCG |
| | CCCAGGGGAAGCACAGCCAGTCCACCAGCTGGTGAAGGAACCTTCAACTCCTGCCTTC |
| | TCGTGGCCCTGGTGGTATGTACTGCTAGGAGCAGTCGTGGCCGCTGCCTGTGTCCTCA |
| | TCTTGGCTCTCTTCCTTGTCCACCGGCGAAAGAAGGAGACCCGTTATGGAGAAGTGTT |
| | TGAACCAACAGTGGAAAGAGGTGAACTGGTAGTCAGGTACCGCGTGCGCAAGTCCTAC |
| | AGAAGCTGCGGGATGTGATGGTGGACCGGCACAAGGTGGCCCTGGGGAAGACTCTGGG |
| | AGAGGGAGAGTTTGGAGCTGTGATGGAAGGCCAGCTCAACCAGGACGACTCCATCCTC |
| | AAGGTGGCTGTGAAGACGATGAAGATTGCCATCTGCACGAGGTCAGAGCTGGAGGATT |
| | TCCTGAGTGAAGCGGTCTGCATGAAGGAATTTGACCATCCCAACGTCATGAGGCTCAT |
| | CGGTGTCTGTTTCCAGGGTTCTGAACGAGAGAGCTTCCCACCACCTGTGGTCATCTTA |
| | CCTTTCATGAAACATGGAGACCTACACAGCTTCCTCCTCTATTCCCGGCTCGGGGGCC |
| | AGCCAGTGTACCTGCCCACTCAGATGCTAGTGAAGTTCATGGCAGACATCGCCAGTGG |
| | CATGGAGTATCTGAGTACCAAGAGATTCATACACCGGGACCTGGCGGCCAGGAACTGC |
| | ATGCTGAATGAGAACATGTCCGTGTGTGTGGCGGACTTCGGGCTCTCCAAGAAGATCT |
| | ACAATGGGGACTACTACCGCCAGGGACGTATCGCCAAGATGCCAGTCAAGTGGATTGC |
| | CATTGAGAGTCTAGCTGACCGTGTCTACACCAGCAAGAGCGATGTGTGGTCCTTCGGG |
| | GTGACAATGTGGGAGATTGCCACAAGAGGCCAAACCCCATATCCGGGCGTGGAGAACA |
| | GGATGGACTGTATGCCTTGATGTCGCGGTGCTGGGAGCTAAATCCCCAGGACCGGCCA |
| | AGTTTTACAGAGCTGCGGGAAGATTTGGAGAACACACTGAAGGCCTTGCCTCCTGCCC |
| | AGGAGCCTGACGAAATCCTCTATGTCAACATGGATGAGGGTGGAGGTTATCCTGAACC |
| | CCCTGGAGCTGCAGGAGGAGCTGACCCCCCAACCCAGCCAGACCCTAAGGATTCCTGT |
| | AGCTGCCTCACTGCGGCTGAGGTCCATCCTGCTGGACGCTATGTCCTCTGCCCTTCCA |
| | CAACCCCTAGCCCCGCTCAGCCTGCTGATAGGGGCTCCCCAGCAGCCCCAGGGCAGGA |
| | GGATGGTGCCTGAGACAACCCTCCACCTGGTACTCCCTCTCAGGATCCAAGCTAAGCA |
| | CTGCCACTGGGGGAAACTCCACCTTCCCACTTTCCCACCCCACGCCTTATCCCCACTT |
| | GCAGCCCTGTCTTCCTACCTATCCCACCTCCATCCCAGACAGGTCCCTGGCCTTCTCT |
| | GTGCAGTAGCATCACCTTGAAAGCAGTAGCATCACCATCTGTAAAAGGAAGGGGTTGG |
| | ATTGCAATATCTGAAGCCCTCCCAGGTGTTAACATTCCAAGACTCTAGAGTCCAAGGT |
| | TTAAAGAGTCTAGATTCAAAGGTTCTAGGTTTCAAAGATGCTGTGAGTCTTTGGTTCT |
| | AAGGACCTGAAATTCCAAAGTCTCTAATTCTATTAAAGTGCTAAGGTTCTAAGGCCTA |
| | CTTTTTTTTTTTTTTTTTTTTTTTTTTTTTTTTTTGCGATAGAGTCTCACTGTGTCAC |
| | CCAGGCTGGAGTGCAGTGGTGCAATCTCGCCTCACTGCAACCTTCACCTACCGAGTTC |
| | AAGTGATTTTCCTGCCTTGGCCTCCCAAGTAGCTGGGATTACAGGTGTGTGCCACCAC |
| | ACCCGGCTAATTTTTATATTTTTAGTAGAGACAGGGTTTCACCATGTTGGCCAGGCTG |
| | GTCTAAAACTCCTGACCTCAAGTGATCTGCCCACCTCAGCCTCCCAAAGTGCTGAGAT |
| | TACAGGCATGAGCCACTGCACTCAACCTTAAGACCTACTGTTCTAAAGCTCTGACATT |
| | ATGTGGTTTTAGATTTTCTGGTTCTAACATTTTTGATAAAGCCTCAAGGTTTTAGGTT |
| | CTAAGTTCTAAGATTCTGATTTTAGGAGCTAAAGGCTCTATGAGTCTAGATGTTTATT |
| | CTTCTAGAGTTCAGAGTCCTTAAAATGTAAGATTATAGATTCTAAAGATTCTATAGTT |
| | CTAGACATGGAGGTTCTAAG |
| | ORF Start ATG at 461 ORF Stop: TGA at 3143 |
| | SEQ ID NO: 164 894 aa MW at 98274.7kD |
| NOV48a, | MAWRCPRMGRVPLAWCLALCGWACMAPRGTQAEESPFVGNPGNTTGARGLTGTLRCQL |
| CG59325-01 | |
| Protein | QVQGEPPEVHWLRDGQILELADSTQTQVPLGEDEQDDWIVVSQLRITSLQLSDTGQYQ |
| Sequence | |
| | CLVFLGHQTFVSQPGYVGLEGLPYFLEEPEDRTVAANTPFNLSCQAQGPPEPVDLLWL |
| | QDAVPLATAPGHGPQRSLHVPGLNKTSSFSCEAHNAKGVTTSRTATITVLPQQPRNLH |
| | LVSRQPTELEVAWTPGLSGIYPLTHCTLQAVLSDDGMGIQAGEPDPPEEPLTSQASVP |
| | PHQLRLGSLHPHPPYHIRVACTSSQGPSSWTHWLPVETPEGVPLGPPENISATRNGSQ |
| | AFVHWQEPRAPLQGTLLGYRLAYQGQDTPEVLMDIGLRQEVTLELQGDGSVSNLTVCV |
| | AAYTAAGDGPWSLPVPLEAWRPGEAQPVHQLVKEPSTPAFSWPWWYVLLGAVVAAACV |
| | LTLALFLVHRRKKETRYGEVFEPTVERGELVVRYRVRKSYSRRTTEATLNSLGISEEL |
| | KEKLRDVMVDRHKVALGKTLGEGEFGAVMEGQLNQDDSILKVAVKTMKIAICTRSELE |
| | DFLSEAVCMKEFDHPNVMRLIGVCFQGSERESFPAPVVILPFMKHGDLHSFLLYSRLG |
| | GQPVYLPTQMLVKFMADTASGMEYLSTKRFTHRDLAARNCMLNENMSVCVADFGLSKK |
| | IYNGDYYRQGRIAKMPVKWIAIESLADRVYTSKSDVWSFGVTMWEIATRGQTPYPGVE |
| | NSEIYDYLRQGNRLKQPADCLDGLYALMSRCWELNPQDRRSFTELREDLENTLKALPP |
| | AQEPDEILYVNMDEGGGYPEPPGAAGGADPPTQRDRKDSCSCLTAAEVHPAGRYVLCP |
| | STTPSPAQPADRGSPAAPGQEDGA |
| | SEQ ID NO: 165 2196 bp |
| NOV48b, | AGCAACTTCTGAGGAAAGTTTGCACCCATGGCGTGGCGGTGCCCCAGGATGGGCAGGG |
| CG59325-03 | |
| DNA | TCCCGCTGGCCTGGTGCTTGGCGCTGTGCGGCTGGGCGTGCATGGCCCCCAGGGGCAC |
| Sequence | |
| | GCAGGCTGAAGAAAGTCCCTTCGTGGGCAACCCAGGGAATATCACAGGTGCCCGTGAG |
| | TCCCCGGGCACCCTTCGGTGTCAGCTCCAGGTTCAGGGAGAGCCCCCCGAGGTACATT |
| | GGCTTCGGGATGGACAGATCCTGGAGCTCGCGGACAGCACCCAGACCCAGGTGCCCCT |
| | GGGTGAGGATGAACAGGATGACTGGATAGTGGTCAGCCAGCTCAGAATCACCTCCCTG |
| | CAGCTTTCCGACACGGGACAGTACCAGTGTTTGGTGTTTCTGGGACATCAGACCTTCG |
| | TGTCCCAGCCTGGCTATGTTGGGCTGGAGGGCTTGCCTTACTTCCTGGAGGAGCCCGA |
| | AGACAGGACTGTGGCCGCCAACACCCCCTTCAACCTGAGCTGCCAAGCTCAGGGACCC |
| | CCAGAGCCCGTGGACCTACTCTGGCTCCAGGATGCTGTCCCCCTGGCCACGGCTCCAG |
| | GTCACGGCCCCCAGCGCAGCCTGCATGTTCCAGGGCTGAACAAGACATCCTCTTTCTC |
| | CTGCGAAGCCCATAACGCCAAGGGGGTCACCACATCCCGCACAGCCACCATCACAGTG |
| | CTCCCCCAGCAGCCCCGTAACCTCCACCTGGTCTCCCACCAGCTGGTGAAGGAATCTT |
| | CAACTCCTGCCTTCTCGTGGCCCTGGTGGTATGTACTGCTAGGAGCAGTCGTGGCCGC |
| | TGCCTGTGTCCTCATCTTGGCTCTCTTCCTTGTCCACCGGCGAAAGAAGGAGACCCGT |
| | TATGGAGAAGTGTTTGAACCAACAGTGGATAGAGGTGAACTGGTAGTCAGGTACCGCG |
| | TGCGCAAGTCCTACAGTCGTCGGACCACTGAAGCTACCTTGAACAGCCTGGGCATCAG |
| | TGAAGAGCTGAAGGAGAAGCTGCGGGATGTGATGGTGGACCGGCACAAGGTGGCCCTG |
| | GGGAAGACTCTGGGAGAGGGAGAGTTTGGAGCTGTGATGGAAGGCCAGCTCAACCAGG |
| | ACGACTCCATCCTCAAGGTGGCTGTGAAGACGATGAAGATTGCCATCTGCACGAGGTC |
| | AGAGCTGGAGGATTTCCTGAGTGAAGCGGTCTGCATGAAAGGAATTTGACCATCCCAC |
| | GTCATGAGGCTCATCGGTGTCTGTTTCCAGGGTTCTGAACGAGAGAGCTTCCCAGCAC |
| | CTGTGGTCATCTTACCTTTCATGAAACATGGAGACCTACACAGCTTCCTCCTCTATTC |
| | CCGGCTCGGGGACCAGCCAGTGTACCTGCCCACTCAGATGCTAGTGAAGTTCATGGCA |
| | GACATCGCCAGTGGCATGGAGTATCTGAGTACCAAGAGATTCATACACCGGGACCTGG |
| | CGGCCAGGAACTGCATGCTGAATGAGAACATGTCCGTGTGTGTGGCGGACTTCGGGCT |
| | CTCCAAGAAGATCTACAATGGGGACTACTACCGCCAGGGACGTATCGCCAAGATGCCA |
| | GTCAAGTGGATTGCCATTGAGAGTCTAGCTGACCGTGTCTACACCAGCAAGAGCGATG |
| | TGTGGTCCTTCGGGGTGACAATGTGGGAGATTGCCACAAGAGGCCAAACCCCATATCC |
| | GGGCGTGGAGAACAGCGAGATTTATGACTATCTGCGCCAGGGAAATCGCCTGAAGCAG |
| | CCTGCGGACTGTCTGGATGGACTGTATGCCTTGATGTCGCGGTGCTGGGAGCTAAATC |
| | CCCAGGACCGGCCAAGTTTTACAGAGCTGCGGGAAGATTTGGAGAACACACTGAAGGC |
| | CTTGCCTCCTGCCCAGGAGCCTGACGAAATCCTCTATGTCAACATGGATGAGGGTGGA |
| | GGTTATCCTGAACCCCCTGGAGCTGCAGGAGGAGCTGACCCCCCAACCCAGCCAGACC |
| | CTAAGGATTCCTGTAGCTGCCTCACTGCGGCTGAGGTCCATCCTGCTGGACGCTATGT |
| | CCTCTGCCCTTCCACAACCCCTAGCCCCGCTCAGCCTGCTGATAGGGGCTCCCCAGCA |
| | GCCCCAGGGCAGGAGGATGGTGCCTGAGACAACCCTCCACCTGGTACTCCCTCTCAGG |
| | ATCCAAGCTAAGCACTGCCACTGGGGAAAACTCCACCTTCCCACTTTCCC |
| | ORF Start: ATG at 28 ORF Stop: TGA at 2113 |
| | SEQ ID NO 166 695 aa MW at 76986.9kD |
| NOV48b | MAWRCPRMGRVPLAWCLALCGWACMAPRGTQAEESPFVGNPGNITGARESPGTLRCQL |
| CG59325-03 | |
| Protein | QVQGEPPEVHWLRDGQTLELADSTQTQVPLGEDEQDDWIVVSQLRITSLQLSDTGQYQ |
| Sequence | |
| | CLVFLGHQTFVSQPGYVGLEGLPYFLEEPEDRTVAANTPFNLSCQAQGPPEPVDLLWL |
| | QDAVPLATAPGHGPQRSLHVPGLNKTSSFSCEAHNAKGVTTSRTATTTVLPQQPRNLH |
| | LVSHQLVKESSTPAFSWPWWYVLLGAVVAAACVLILALFLVHRRKKETRYGEVFEPTV |
| | DRGELVVRYRVRKSYSRRTTEATLNSLGISEELKEKLRDVMVDRHKVALGKTLGEGEF |
| | GAVMEGQLNQDDSILKVAVKTMKTAICTRSELEDFLSEAVCMKEFDHPNVMRLIGVCF |
| | QGSERESFPAPVVILPFMKHGDLHSFLLYSRLGDQPVYLPTQMLVKFMADIASGMEYL |
| | STKRFIHRDLAARNCMLNENMSVCVADFGLSKKTYNGDYYRQGRIAKMPVKWIAIESL |
| | ADRVYTSKSDVWSFGVTMWEIATRGQTPYPGVENSEIYDYLRQGNRLKQPADCLDGLY |
| | ALMSRCWELNPQDRPSFTELREDLENTLKALPPAQEPDEILYVNMDEGGGYPEPPGAA |
| | GGADPPTQPDPKDSCSCLTAAEVHPAGRYVLCPSTTPSPAQPADRGSPAAPGQEDGA |
| | SEQ ID NO: 167 13999 bp |
| NOV48c, | GAGTGGAGTTCTGGAGGAATGTTTACCAGACACAGAGCCCAGAGGGACAGCGCCCAGA |
| CG59325-04 | |
| DNA | GCCCAGATAGAGAGACACGGCCTCACTGGCTCAGCACCAGGGTCCCCTTCCCCCTCCT |
| Sequence | |
| | CAGCTCCCTCCCTGGCCCCTTTAAGAAAGAGCTGATCCTCTCCTCTCTTGAGTTAACC |
| | CCTGATTGTCCAGGTGGCCCCTGGCTCTGGCCTGGTGGGCGGAGGCAAAGGGGGAGCC |
| | AGGGGCGGAGAAAGGGTTGCCCAAGTCTGGGAGTGAGGGAAGGAGGCAGGGGTGCTGA |
| | GAAGGCGGCTGCTGGGCAGAGCCGGTGGCAAGGGCCTCCCCTGCCGCTGTGCCAGGCA |
| | GGCAGTGCCAAATCCGGGGAGCCTGGAGCTGGGGGGAGGGCCGGGGACAGCCCGGCCC |
| | GCTGCCCCCTCCCCCGCTGGGAGCCCAGCAACTTCTGAGGAAAGTTTGGCACCCATGG |
| | CGTGGCGGTGCCCCAGGATGGGCAGGGTCCCGCTGGCCTGGTGCTTGGCGCTGTGCGG |
| | CTGGGCGTGCATGGCCCCCAGGGGCACGCAGGCTGAAGAAAGTCCCTTCGTGGGCAAC |
| | CCAGGGAATATCACAGGTGCCCGGGGACTCACGGGCACCCTTCGGTGTCAGCTCCAGG |
| | TTCAGGGAGAGCCCCCCGAGGTACATTGGCTTCGGGATGGACAGATCCTGGAGCTCGC |
| | GGACAGCACCCAGACCCAGGTGCCCCTGGGTGAGGATGAACAGGATGACTGGATAGTG |
| | GTCAGCCAGCTCAGAATCACCTCCCTGCAGCTTTCCGACACGGGACAGTACCAGTGTT |
| | TGGTGTTTCTGGGACATCAGACCTTCGTGTCCCAGCCTGGCTATGTTGGGCTGGAGGG |
| | CTTGCCTTACTTCCTGGAGGAGCCCGAAGACAGGACTGTGGCCGCCAACACCCCCTTC |
| | AACCTGAGCTGCCAAGCTCAGGGACCCCCAGAGCCCGTGGACCTACTCTGGCTCCAGG |
| | ATGCTGTCCCCCTGGCCACGGCTCCAGGTCACGGCCCCCAGCGCAGCCTGCATGTTCC |
| | AGTGCTCCCCCAGCAGCCCCGTAACCTCCACCTGGTCTCCCGCCAACCCACGGAGCTG |
| | GAGGTGGCTTGGACTCCAGGCCTGAGCGGCATCTACCCCCTGACCCACTGCACCCTGC |
| | AGGCTGTGCTGTCAGACGATGGGATGGGCATCCAGGCGGGAGAACCAGACCCCCCAGA |
| | GGAGCCCCTCACCTCGCAAGCATCCGTGCCCCCCCATCAGCTTCGGCTAGGCAGCCTC |
| | CATCCTCACCCCCCTTATCACATCCGCGTGGCATGCACCAGCAGCCAGGGCCCCTCAT |
| | CCTGGACCCACTGGCTTCCTGTGGAGACGCCGGAGGGAGTGCCCCTGGGCCCCCCTGA |
| | GAACATTAGTGCTACGCGGAATGGGAGCCAGGCCTTCGTGCATTGGCAAGAGCCCCGG |
| | GCGCCCCTGCAGGGTACCCTGTTAGGGTACCGGCTGGCGTATCAAGGCCAGGACACCC |
| | CAGAGGTGCTAATGGACATAGGGCTAAGGCAAGAGGTGACCCTGGAGCTGCAGGGGGA |
| | CGGGTCTGTGTCCAATCTGACAGTGTGTGTGGCAGCCTACACTGCTGCTGGGGATGGA |
| | CCCTGGAGCCTCCCAGTACCCCTGGAGGCCTGGCGCCCAGGGGAAGCACAGCCAGTCC |
| | ACCAGCTGGTGAAGGAACCTTCAACTCCTGCCTTCTCGTGGCCCTGGTGGTATGTACT |
| | GCTAGGAGCAGTCGTGGCCGCTGCCTGTGTCCTCATCTTGGCTCTCTTCCTTGTCCAC |
| | CGGCGAAAGAAGGAGACCCGTTATGGAGAAGTGTTTGAACCAACAGTGGAAAGAGGTG |
| | CTTGAACAGCCTGGGCATCAGTGAAGAGCTGAAGGAGAAGCTGCGGGATGTGATGGTG |
| | GACCGGCACAAGGTGGCCCTGGGGAAGACTCTGGGAGAGGGAGAGTTTGGAGCTGTGA |
| | TGGAAGGCCAGCTCAACCAGGACGACTCCATCCTCAAGGTGGCTGTGAAGACGATGAA |
| | GATTGCCATCTGCACGAGGTCAGAGCTGGAGGATTTCCTGAGTGAAGCGGTCTGCATG |
| | AAGGAATTTGACCATCCCAACGTCATGAGGCTCATCGGTGTCTGTTTCCAGGGTTCTG |
| | AACGAGAGAGCTTCCCAGCACCTGTGGTCATCTTACCTTTCATGAAACATGGAGACCT |
| | ACACAGCTTCCTCCTCTATTCCCGGCTCGGGGGCCAGCCAGTGTACCTGCCCACTCAG |
| | ATGCTAGTGAAGTTCATGGCAGACATCGCCAGTGGCATGGAGTATCTGAGTACCAAGA |
| | GATTCATACACCGGGACCTGGCGGCCAGGAACTGCATGCTGAATGAGAACATGTCCGT |
| | GTGTGTGGCGGACTTCGGGCTCTCCAAGAAGATCTACAATGGGGACTACTACCGCCAG |
| | GGACGTATCGCCAAGATGCCAGTCAAGTGGATTGCCATTGAGAGTCTAGCTGACCGTG |
| | TCTACACCAGCAAGAGCGATGTGTGGTCCTTCGGGGTGACAATGTGGGAGATTGCCAC |
| | AAGAGGCCAAACCCCATATCCGGGCCTGGAGAACAGCGAGATTTATGACTATCTGCGC |
| | CAGGGAAATCGCCTGAAGCAGCCTGCGGACTGTCTGGATGGACTGTATGCCTTGATGT |
| | CGCGGTGCTGGGAGCTAAATCCCCAGGACCGGCCAAGTTTTACAGAGCTGCGGGAAGA |
| | TTTGGAGAACACACTGAAGGCCTTGCCTCCTGCCCAGGAGCCTGACGAAATCCTCTAT |
| | GTCAACATGGATGAGGGTGGAGGTTATCCTGAACCCCCTGGAGCTGCAGGAGGAGCTG |
| | ACCCCCCAACCCAGCCAGACCCTAAGGATTCCTGTAGCTGCCTCACTGCGGCTGAGGT |
| | CCATCCTGCTGGACGCTATGTCCTCTGCCCTTCCACAACCCCTAGCCCCGCTCAGCCT |
| | GCTGATAGGGGCTCCCCAGCAGCCCCAGGGCAGGAGGATGGTGCCTGAGACAACCCTC |
| | CACCTGGTACTCCCTCTCAGGATCCAAGCTAAGCACTGCCACTGGGGGAAACTCCACC |
| | TTCCCACTTTCCCACCCCACGCCTTATCCCCACTTGCAGCCCTGTCTTCCTACCTATC |
| | CCACCTCCATCCCAGACAGGTCCCTGGCCTTCTCTGTGCAGTAGCATCACCTTGAAAG |
| | CAGTAGCATCACCATCTGTAAAAGGAAGGGGTTGGATTGCAATATCTGAAGCCCTCCC |
| | AGGTGTTAACATTCCAAGACTCTAGAGTCCAAGGTTTAAAGAGTCTAGATTCAAAGGT |
| | TCTAGGTTTCAAAGATGCTGTGAGTCTTTGGTTCTAAGGACCTGAAATTCCAAAGTCT |
| | CTAATTCTATTAAAGTGCTAAGGTTCTAAGGCCTACTTTTTTTTTTTTTTTTTTTTTT |
| | TTTTTTTTTTTTGCGATAGAGTCTCACTGTGTCACCCAGGCTGGAGTGCAGTGGTGCA |
| | ATCTCGCCTCACTGCAACCTTCACCTACCGAGTTCAAGTGATTTTCCTGCCTTGGCCT |
| | CCCAAGTAGCTGGGATTACAGGTGTGTGCCACCACACCCGGCTAATTTTTATATTTTT |
| | AGTAGAGACAGGGTTTCACCATGTTGGCCAGGCTGGTCTAAAACTCCTGACCTCAAGT |
| | GATCTGCCCACCTCAGCCTCCCAAAGTGCTGAGATTACAGGCATGAGCCACTGCACTC |
| | AACCTTAAGACCTACTGTTCTAAAGCTCTGACATTATGTGGTTTTAGATTTTCTGGTT |
| | CTAACATTTTTGATAAAGCCTCAAGGTTTTAGGTTCTAAAGTTCTAAGATTCTGATTT |
| | TAGGAGCTAAGGCTCTATGAGTCTAGATGTTTATTCTTCTAGAGTTCAGAGTCCTTAA |
| | AATGTAAGATTATAGATTCTAAAGATTCTATAGTTCTAGACATGGAGGTTCTAAG |
| | ORF Start: ATG at 461 ORF Stop. TGA at 3062 |
| | SEQ ID NO: 168 867 aa MW at 95509.6kD |
| NOV48c, | MAWRCPRMGRVPLAWCLALCGWACMAPRGTQAEESPFVGNPGNITGARGLTGTLRCQL |
| CG59325-04 | |
| Protein | QVQGEPPEVHWLRDGQILELADSTQTQVPLGEDEQDDWIVVSQLRITSLQLSDTGQYQ |
| Sequence | |
| | CLVFLGHQTFVSQPGYVGLEGLPYFLEEPEDRTVAANTPFNLSCQAQGPPEPVDLLWL |
| | QDAVPLATAPGHGPQRSLHVPVLPQQPRNLHLVSRQPTELEVAWTPGLSGIYRLTHCT |
| | LQAVLSDDGMGIQAGEPDPPEEPLTSQA3VPPHQLRLGSLHPHPPYHIRVACTSSQGP |
| | SSWTHWLPVETPEGVPLGPRENISATRNGSQAFVHWQEPRAPLQGTLLGYRLAYQGQD |
| | TPEVLMDIGLRQEVTLELQGDGSVSNLTVCVAAYTAAGDGPWSLPVPLEAWRPGEAQP |
| | VHQLVKERSTPAFSWPWWYVLLGAVVAAACVLILALFLVHRRKKETRYGEVFBPTVER |
| | GELVVRYRVRKSYSRRTTEATLNSLGISEELKEKLRDVMVDRHKVALGKTLGEGEFGA |
| | VMEGQLNQDDSILKVAVKTMKTAICTRSELEDFLSEAVCMKEPDHPNVMRLIGVCFQG |
| | SERESFPAPVVILPFMKHGDLHSFLLYSRLGGQPVYLPTQMLVKFMADIASGMEYLST |
| | KRFIHRDLAARNCMLNENMSVCVADFGLSKKIYNGDYYRQGRIAKMPVKWIAIESLAD |
| | RVYTSKSDVWSFGVTMWEIATRGQTPYPGVENSEIYDYLRQGNRLKQPADCLDGLYAL |
| | MSRCWELNPQDRPSFTELREDLENTLKALPPAQEPDEILYVNMDEGGGYPEPPGAAGG |
| | ADPPTQPDPKDSCSCLTAAEVHPAGRYVLCPSTTPSPAQPADRGSPAAPGQEDGA |
| | SEQ ID NO: 169 1245 bp |
| NOV48d, | AAGCTTGAAGAAAGTCCCTTCGTGGGCAACCCAGGGAATATCACAGGTGCCCGGGGAC |
| 72557413 | |
| DNA | TCACGGGCACCCTTCGGTGTCAGCTCCAGGTTCAGGGAGAGCCCCCCGAGGTACATTG |
| Sequence | |
| | GCTTCGGGATGGACAGATCCTGGAGCTCGCGGACAGCACCCAGACCCAGGTGCCCCTG |
| | GGTGAGGGTGAACAGGATGACTGGATAGTGGTCAGCCAGCTCAGAATCACCTCCCTGC |
| | AGCTTTCCGACACGGGACAGTACCAGTGTTTGGTGTTTCTGGGACATCAGACCTTCGT |
| | GTCCCAGCCTGGCTATGTTGGGCTGGAGGGCTTGCCTTACTTCCTGGAGGAGCCCGAA |
| | GACAGGACTGTGGCCGCCAACACCCCCTTCAACCTGAGCTGCCAAGCTCAGGGACCCC |
| | CAGACCCCGTGGACCTACTCTGGCTCCAGGATGCTGTCCCCCTGGCCACCGCTCCAGG |
| | TCACGCCCCCCAGCGCAGCCTGCATGTTCCAGGGCTGAACAAGACATCCTCTTTCTCC |
| | TCCCCCAGCAGCCCCGTAACCTCCACCTGGTCTCCCGCCAACCCACGGAGCTGGAGGT |
| | CCCTCACCTCGCAAGCATCCGTGCCCCCCCATCAGCTTCGGCTAGGCAGCCTCCATCC |
| | TCACACCCCTTATCACATCCGCGCGGCATGCACCAGCAGCCAGGGCCCCTCATCCTGG |
| | ACCCACTGGCTTCCTGTGGAGACGCCGGAGGGAGTGCCCCTGGGCCCCCCTGAGAACA |
| | TTAGTGCTACGCGGAATGGGAGCCAGGCCTTCGTGCATTGGCAAGAGCCCCGGGCGCC |
| | CCTGCAGGGTACCCTGTTAGGGTACCGGCTGGCGTATCAAGGCCAGGACACCCCAGAG |
| | GTGCTAATGGACATAGGGCTAAGGCAAGAGGTGACCCTGGAGCTGCAGGGGGACGGGT |
| | CTGTGTCCAATCTGACAGTGTGTGTGGCAGCCTACACTGCTGCTGGGGATGGACCCTG |
| | GAGCCTCCCAGTACCCCTGGAGGCCTGGCGCCCAGTGAAGGAACCTTCAACTCCTGCC |
| | TTCTCGTGGCCCTGGTGGTATCTCGAG |
| | ORF Start at 1 ORF Stop: end of sequence |
| | SEQ ID NO: 170 415 aa MW at 45089.2kD |
| NOV48d, | KLEESPFVGNPGNITGARGLTGTLRCQLQVQGEPPEVHWLRDGQILELADSTQTQVPL |
| 17557413 | |
| Protein | GEGEQDDWIVVSQLRITSLQLSDTGQYQCLVFLGHQTFVSQPGYVGLEGLRYFLEERE |
| Sequence | |
| | DRTVAANTRFNLSCQAQGRPEPVDLLWLQDAVPLATAPGHGPQRSLHVPGLNKTSSFS |
| | CEAHNAKGVTTSRTATITVLPQQPRNLHLVSRQPTELEVAWTPGLSGIYPLTHCTLQA |
| | VLSDDGMGIQAGELDPPEFPLTSQASVPPHQLRLGSLHPHTPYHIRAACTSSQGPSSW |
| | THWLPVETPEGVPLGPRENISATRNGSQAFVHWQFPRAPLQGTLLGYRLAYQGQDTPE |
| | VLMDTGLRQEVTLELQGDGSVSNLTVCVAAYTAAGDGRWSLPVRLEAWRPVKEPSTPA |
| | FSWPWWYLE |
| | SEQ ID NO: 171 1191 bp |
| NOV48c, | AAGCTTGAAGAAAGTCCCTTCGTGGGCAACCCAGGGAATATCACAGGTGCCCGGGGAC |
| 172557493 | |
| DNA | TCACGGGCACCCTTCGGTGTCAGCTCCAGGTTCAGGGAGAGCCCCCCGAGGTACATTG |
| Sequence | |
| | GCTTCGGGATGGACAGATCCTGGAGCTCGCGGACAGCACCCAGACCCAGGTGCCCCTG |
| | GGTGAGGATGAACAGGATGACTGGATAGTGGTCAGCCAGCTCAGAATCACCTCCCTGC |
| | AGCTTTCCGACACGGGACAGTACCAGTGTTTGGTGTTTCTGGGACATCAGACCTTCGT |
| | GTCCCAGCCTGGCTATGTTGGGCTGGACGGCTTGCCTTACTTCCTGGAGGAGCCCGAA |
| | GACAGGACTGTGGCCGCCAACACCCCCTTCAACCTGAGCTGCCAAGCTCAGGGACCCC |
| | CAGAGCCCGTGGACCTACTCTGGCTCCAGGATGCTGTCCCCCTGGCCACGGCTCCAGG |
| | TCACGGCCCCCAGCGCAGCCTGCATGTTCCAGTGCTCCCCCAGCAGCCCCGTAACCTC |
| | CACCTGGTCTCCCGCCAACCCACGGAGCTGGAGGTGGCTTGGACTCCAGGCCTGAGCG |
| | GCATCTACCCCCTGACCCACTGCACCCTGCAGGCTGTGCTGTCAGACGATGGGATGGG |
| | CATCCAGGCGGGAGAACCAGACCCCCCAGAGGAGCCCCTCACCTCGCAAGCATCCGTG |
| | CCCCCCCATCAGCTTCGGCTAGGCAGCCTCCATCCTCACACCCCTTATCACATCCGCG |
| | TGGCATGCACCAGCAGCCAGGGCCCCTCATCCTGGACCCACTGGCTTCCTGTGGAGAC |
| | GCCGGAGGGAGTGCCCCTGGGCCCCCCTGAGAACATTAGTGCTACGCGGAATGGGAGC |
| | ACCGGCTGGCGTATCAAGGCCAGGACACCCCAGAGGTGCTAATGGACATAGGGCTAAG |
| | GCAAGAGGTGACCCTGGAGCTGCAGGGGGACGGGTCTGTGTCCAATCTGACAGTGCGT |
| | GTGGCAGCCTACACTGCTGCTGGGGATGGACCCTGGAGCCTCCCAGTACCCCTGGAGG |
| | CCTGGCGCCCAGGGCTAGCACAGCCAGTCCACCAGCTGGTGAAGGAACCTTCAACTCC |
| | TGCCTTCTCGTGGCCCTGGTGGTATCTCGAG |
| | ORF Start at 1 ORF Stop: end of sequence |
| | SEQ ID NO: 172 397 aa MW at 43406.3kD |
| NOV48e, | KLEESPFVGNPGNITGARGLTGTLRCQLQVQGEPPEVHWLRDGQILELADSTQTQVPL |
| 172557493 | |
| Protein | GEDEQDDWIVVSQLRITSLQLSDTGQYQCLVFLGHQTFVSQPGYVGLEGLPYFLEEPE |
| Sequence | |
| | DRTVAANTPFNLSCQAQGPPEPVDLLWLQDAVPLATAPGHGPQRSLHVPVLPQQPRNL |
| | HLVSRQPTELEVAWTPGLSGIYPLTHCTLQAVLSDDGMGTQAGEPDPPEEPLTSQASV |
| | PPHQLRLGSLHPHTPYHIRVACTSSQGPSSWTHWLPVETPEGVPLGPPENISATRNGS |
| | QAFVHWQEPRAPLQGTLLGYRLAYQGQDTPEVLMDIGLRQEVTLELQGDGSVSNLTVR |
| | VAAYTAAGDGPWSLPVPLEAWRPGQAQPVHQLVKEPSTPAFSWPWWYLE |
| | SEQ ID NO: 173 1272 bp |
| NOV48f | AAGCTTGAAGAAAGTCCCTTCGTGGGCAACCCAGGGAATATCACAGGTGCCCGTGAGT |
| 172557606 | |
| DNA | CCCCGGGCACCCTTCGGTGTCAGCTCCAGGTTCAGGGAGAGCCCCCCGAGGTACATTG |
| Sequence | |
| | GCTTCGGGATGGACAGATCCTGGAGCTCGCGGACAGCACCCAGACCCAGGTGCCCCTG |
| | GGTGAGGATGAACAGGATGACTGGATAGTGGTCAGCCAGCTCAGAATCACCTCCCTGC |
| | AGCTTTCCGACACGGGACAGTACCAGTGTTTGGTGTTTCTGGGACATCAGACCTTCGT |
| | GTCCCAGCCTGGCTATGTTGGGCTGGAGGGCTTGCCTTACTTCCTGGAGGAGCCCGAA |
| | GACAGGACTGTGGCCGCCAACACCCCCTTCAACCTGAGCTGCCAAGCTCAGGGACCCC |
| | CAGAGCCCGTGGACCTACTCTGGCTCCAGGATGCTGTCCCCCTGGCCACGGCTCCAGG |
| | TCACGGCCCCCAGCGCAGCCTGCATGTTCCAGGGCTGAACAAGACATCCTCTTTCTCC |
| | TGCGAAGCCCATAACGCCAAGGGGGTCACCACATCCCGCACAGCCACCATCACAGTGC |
| | TCCCCCAGCAGCCCCGTAACCTCCACCTGGTCTCCCGCCAACCCACGGAGCTGGAGGT |
| | GGCTTGGACTCCAGGCCTGAGCGGCATCTACCCCCTGACCCACTGCACCCTGCAGGCT |
| | GTGCTGTCAGACGATGGGATGGGCATCCAGGCGGGAGAACCAGACCCCCCAGAGGAGC |
| | CCCTCACCTCGCAAGCATCCGTGCCCCCCCATCAGCTTCGGCTAGGCAGCCTCCATCC |
| | TCACACCCCTTATCACATCCGCGTGGCATGCACCAGCAGCCAGGGCCCCTCATCCTGG |
| | ACCCACTGGCTTCCTGTGGAGACGCCGGAGGGAGTGCCCCTGGGCCCCCCTGAGAACA |
| | TTAGTGCTACGCGGAATGGGAGCCAGGCCTTCGTGCATTGGCAAGAGCCCCGGGCGCC |
| | CCTGCAGGGTACCCTGTTAGGGTACCGGCTGGCGTATCAAGGCCAGGACACCCCAGAG |
| | GTGCTAATGGACATAGGGCTAAGGCAAGAGGTGACCCTGGAGCTGCAGGGGGACGGGT |
| | CTGTGTCCAATCTGACAGTGTGTGTGGCAGCCTACACTGCTGCTGGGGATGGACCCTG |
| | GAGCCTCCCAGTACCCCTGGAGGCCTGGCGCCCAGGGCAAGCACAGCCAGTCCACCAG |
| | CTGGTGAAGGAACCTTCAACTCCTGCCTTCTCGTGGCCCTGGTGGTATCTCGAG |
| | ORF Start: at 1 ORF Stop: end of sequence |
| | SEQ ID NO: 174 424 aa MW at 46160.3kD |
| NOV48f, | KLEESPFVGNPGNITGARESPGTLRCQLQVQGEPPEVHWLRDGQILELADSTQTQVPL |
| 172557606 | |
| Protein | GEDEQDDWIVVSQLRITSLQLSDTGQYQCLVFLGHQTFVSQPGYVGLEGLPYFLEERE |
| Sequence | |
| | DRTVAANTPFNLSCQAQGPPEPVDLLWLQDAVPLATAPGHGPQRSLHVPGLNKTSSFS |
| | CEAHNAKGVTTSRTATITVLPQQPRNLHLVSRQPTELEVAWTPGLSGIYPLTHCTLQA |
| | THWLPVETPEGVPLGPPENISATRNGSQAFVHWQEPRAPLQGTLLGYRLAYQGQDTPE |
| | VLMDIGLRQEVTLELQGDGSVSNLTVCVAAYTAAGDGPWSLPVPLEAWRPGQAQPVHQ |

Sequence comparison of the above protein sequences yields the following sequence relationships shown in Table 48B.

TABLE 48B
Comparison of NOV48a against NOV48b through NOV48f

| | NOV48a Residues/ | Identities/Similarities |
|---|---|---|
| Protein Sequence | Match Residues | for the Matched Region |
| NOV48b | 435 . . . 894 | 400/460 (86%) |
| | 236 . . . 695 | 401/460 (86%) |
| NOV48c | 1 . . . .894 | 810/894 (90%) |
| | 1 . . . .867 | 810/894 (90%) |
| NOV48d | 33 . . . 453 | 407/421 (96%) |
| | 3 . . . 414 | 408/421 (96%) |
| NOV48e | 33 . . . 453 | 390/421 (92%) |
| | 3 . . . 396 | 392/421 (92%) |
| NOV48f | 33 . . . 453 | 415/421 (98%) |
| | 3 . . . 423 | 417/421 (98%) |

Further analysis of the NOV48a protein yielded the following properties shown in Table 48C.

TABLE 48C
Protein Sequence Properties NOV48a

| | |
|---|---|
| PSort | 0.4600 probability located in plasma membrane; 0.1129 |
| analysis: | probability located in microbody (peroxisome); 0.1000 |
| | probability located in endoplasmic reticulum (membrane); |
| | 0.1000 probability located in endoplasmic reticulum (lumen) |
| SignalP | Cleavage site between residues 33 and 34 |
| analysis: | |

A search of the NOV48a protein against the Geneseq database, a proprietary database that contains sequences published in patents and patent publication, yielded several homologous proteins shown in Table 48D.

TABLE 48D
Geneseq Results for NOV48a

| | | | Identities/ | |
|---|---|---|---|---|
| | | | Similari- | |
| | | NOV48a | ties | |
| | Protein/ | Residues/ | for the | |
| Geneseq | Organism/Length | Match | Matched | Expect |
| Identifier | [Patent #, Date] | Residues | Region | Value |
| AAB90763 | Human shear stress- | 1 . . . 894 | 894/894 | 0.0 |
| | response protein | 1 . . . 894 | (100%) | |
| | SEQ ID NO: 26 - | | 894/894 | |
| | Homo sapiens, | | (100%) | |
| | 894 aa. | | | |
| | [WO200125427-A1, | | | |
| | 12 APR. 2001] | | | |
| AAR85753 | Human axl receptor - | 1 . . . 894 | 891/894 | 0.0 |
| | Homo sapiens, | 1 . . . 894 | (99%) | |
| | 894 aa. | | 892/894 | |
| | [U.S. Pat. 5468634-A, | | (99%) | |
| | 21 NOV. 1995] | | | |
| ABG22182 | Novel human | 1 . . . 894 | 887/895 | 0.0 |
| | diagnostic protein | 53 . . . 947 | (99%) | |
| | #22173 -Homo | | 890/895 | |
| | sapiens, | | (99%) | |
| | 947 aa. | | | |
| | [WO200175067-A2, | | | |
| | 11 OCT. 2001] | | | |
| ABG22182 | Novel human | 1 . . . 894 | 887/895 | 0.0 |
| | diagnostic protein | 53 . . . 947 | (99%) | |
| | #22173 -Homo | | 890/895 | |
| | sapiens, | | (99%) | |
| | 947 aa. | | | |
| | [WO200175067-A2, | | | |
| | 11 OCT. 2001] | | | |
| AAU84262 | Human endometrial | 1 . . . .894 | 882/894 | 0.0 |
| | cancer related | 1 . . . .885 | (98%) | |
| | protein, AXL - | | 883/894 | |
| | Homo sapiens, 885 aa. | | (98%) | |
| | [WO200209573-A2, | | | |
| | 07 FEB. 2002] | | | |

In a BLAST search of public sequence datbases, the NOV48a protein was found to have homology to the proteins shown in the BLASTP data in Table 48E.

TABLE 48E
Public BLASTP Results for NOV48a

| | | | Identities/ | |
|---|---|---|---|---|
| | | | Similari- | |
| | | NOV48a | ties | |
| Protein | | Residues/ | for the | |
| Accession | Protein/ | Match | Matched | Expect |
| Number | Organism/Length | Residues | Portion | Value |
| A41527 | protein-tyrosine | 1 . . . 894 | 891/894 | 0.0 |
| | kinese | 1 . . . 894 | (99%) | |
| | (EC 2.7.1.112) axl | | 892/894 | |
| | precursor, major | | (99%) | |
| | splice form - | | | |
| | human, 894 aa. | | | |
| P30530 | Tyrosine-protein | 8 . . . 894 | 887/887 | 0.0 |
| | kinase receptor | 1 . . . 887 | (100%) | |
| | UFO precursor | | 887/887 | |
| | (EC 2.7.1.112) | | (100%) | |
| | (AXL oncogene) - | | | |
| | Homo sapiens | | | |
| | (Human), 887 aa. | | | |
| Q8V1A0 | Rat Axl longform - | 8 . . . 894 | 781/888 | 0.0 |
| | Rattus norvegicus | 1 . . . 888 | (87%) | |
| | (Rat), 888 aa. | | 816/888 | |
| | | | (90%) | |
| Q00993 | Tyrosine-protein | 8 . . . 894 | 779/888 | 0.0 |
| | kinase receptor | 1 . . . 888 | (87%) | |
| | UFO precursor | | 814/888 | |
| | (EC 2.7.1.112) | | (90%) | |
| | (Adhesion-related | | | |
| | kinase) - Mus | | | |
| | musculus (Mouse), | | | |
| | 888 aa. | | | |
| Q8V199 | Rat Axl shortform - | 8 . . . 894 | 776/888 | 0.0 |
| | Rattus norvegicus | 1 . . . 879 | (87%) | |
| | (Rat). 879 aa. | | 809/888 | |
| | | | (90%) | |

PFam analysis predicts that the NOV48a protein contains the domains shown in the Table 48F.

TABLE 48F
Domain Analysis of NOV48a

| | NOV48a | Identities/Similarities | Expect |
|---|---|---|---|
| Pfam Domain | Match Region | for the Matched Region | Value |
| ig | 49 . . . 119 | 18/73(25%) | 1.2e-07 |
| | | 48/73(66%) | |
| ig | 153 . . . 207 | 8/59(14%) | 0.053 |
| | | 37/59(63%) | |
| fn3 | 225 . . . 321 | 21/100(21%) | 7.6e-05 |
| | | 68/100(68%) | |
| fn3 | 334 . . . 418 | 20/87(23%) | 5.1e-10 |
| | | 62/87(71%) | |
| pkinase | 536 . . . 803 | 80/303 (26%) | 1.9e-71 |
| | | 212/303 (70%) | |

### Example 49

The NOV49 clone was analyzed, and the nucleotide and encoded polypeptide sequences are shown in Table 49A.

TABLE 49A
NOV49 Sequence Analysis

| | |
|---|---|
| | SEQ ID NO 175 406 bp |
| NOV49a. | GGAAGATGGCGGAACAGGCTACCAAGTCCGTGCTGTTTGTGTGTCTGGGTAACATTTG |
| CG59582-03 | |
| DNA | TCGATCACCCATTGCAGAAGCAGTTTTCAGGAAACTTGTAACCGATCAAAACATCTCA |
| Sequence | |
| | GAGAATATTACCAAAGAAGATTTTGCCACATTTGATTATATACTATGTATGGATGAAA |
| | GCAATCTGAGAGATTTGAATAGAAAAAGTAATCAAGTTAAAACCTGCAAAGCTAAAAT |
| | TGAACTACTTGGGAGCTATGATCCACATAAACAACTTATTATTGAAGATCCCTATTAT |
| | GGGAATGACTCTGACTTTGAGACGGTGTACCAGCAGTGTGTCAGGTGCTGCAGAGCGT |
| | TCTTGGAGAAGGCCCACTGAGGCAGGTTCGTGCCCTGCTGCGGCCAGCCTGACTAGAC |
| | ORF Start: at 9 ORF Stop: TGA at 366 |
| | SEQ ID NO: 176 119 aa MW at 13669.4kD |
| NOV49a. | AEQATKSVLFVCLGNICRSRIAEAVFRKLVTDQNISENITKEDFATFDYILCMDESNL |
| CG59582-03 | |
| Protein | RDLNRKSNQVKTCKAKIELLGSYDPQKQLIIEDPYYGNDSDPETVYQQCVRCCRAFLE |
| Sequence | |
| | KAH |

Further analysis of the NOV49a protein yielded the following properties shown in Table 49B.

TABLE 49B
Protein Sequence Properties NOV49a

| | |
|---|---|
| PSort | 0.5500 probability located in endoplasmic reticulum |
| analysis | (membrane); 0.1900 probability |
| | located in lysosome (lumen). |
| | 0.1000 probability located in endoplasmic reticulum |
| | (lumen); 0.1000 probability located in outside |
| SignalP | Cleavage site between residues 25 and 26 |
| analysis: | |

A search of the NOV49a protein against the Geneseq database, a proprietary database that contains sequences published in patents and patent publication, yielded several homologous proteins shown in Table 49C.

TABLE 49C
Geneseq Results for NOV49a

| | | NOV49a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAU30795 | Novel human secreted protein #1286- | 1 . . . 119 | 103/170(60%) | 1e-43 |
| | Homo sapiens, 246 aa. [WO200179449- | 77 . . . 246 | 106/170(61%) | |
| | A2, 25-OCT-2001] | | | |
| AAU30794 | Novel human secreted protein #1285 - | 15 . . . 87 | 55/90(61%) | 3e-20 |
| | Homo sapiens, 102 aa. [WO200179449- | 13 . . . 102 | 61/90(67%) | |
| | A2, 25-OCT-2001] | | | |
| AAE05979 | Zygosaceharomyces rouxii PPPase 2 | 7 . . . 116 | 55/152(36%) | 2e-18 |
| | protein-Zygosaceharomyces rouxii, 160 | 8 . . . 157 | 74/152(48%) | |
| | aa. [WO200 153306-A2, 26-JUL-2001] | | | |
| AAE05978 | Zygosaccharomyces rouxii PPPase 1 | 7 . . . 116 | 53/152(34%) | 3e-17 |
| | protein-Zygosaccharomyces | 8 . . . 157 | 73/152(47%) | |
| | rouxii, 160 aa. [WO200153306-A2, | | | |
| | 26-JUL-2001] | | | |
| ABB71773 | Drosophila melanogaster polypeptide | 4 . . . 94 | 47/132(35%) | 1e-11 |
| | SEQ ID NO 42111 - Drosophila | 293 . . . 422 | 63/132(47%) | |
| | melanogaster, 424 aa. [WO200171042- | | | |
| | A2. 27-SEP-2001] | | | |

In a BLAST search of public sequence datbases, the NOV49a protein was found to have homology to the proteins shown in the BLASTP data in Table 49D.

TABLE 49D
Public BLASTP Results for NOV49a

| | | NOV49a | Identities/ | |
|---|---|---|---|---|
| Protein | | Residues/ | Similarities for | |
| Accession | | Match | the Matched | Expect |
| Number | Protein/Organism/Length | Residues | Portion | Value |
| A38148 | protein-tyrosine-phosphatase(EC | 1 . . . 119 | 119/157(75%) | 1e-59 |
| | 3.1 3.48), low molecular weight, splice | 2 . . . 158 | 119/157(75%) | |
| | form f [validated] - human. 158 aa. | | | |
| AAH07422 | Acid phosphatase 1, soluble - Homo | 1 . . . 119 | 119/157(75%) | 1e-59 |
| | sapiens(Human), 158 aa. | 2 . . . 158 | 119/157(75%) | |
| P24667 | Red cell acid phosphatase 1, isozyme S | 1 . . . 119 | 119/157(75%) | 1e-59 |
| | (EC 3.1.3.2)(ACPI)(Low molecular | 1 . . . 157 | 119/157(75%) | |
| | weight phosphotyrosine protein | | | |
| | phosphatase)(EC 3.1.3.48)(Adipocyte | | | |
| | acid phosphatase, isozyme beta) - Homo | | | |
| | sapiens(Human), 157 aa. | | | |
| P24666 | Red cell acid phosphatase 1, isozyme F | 1 . . . 119 | 119/157(75%) | 1e-59 |
| | (EC 3.1.3.2)(ACPI)(Low molecular | 1 . . . 157 | 119/157(75%) | |
| | weight phosphotyrosine protein | | | |
| | phosphatase)(EC 3.1.3.48)(Adipocyte | | | |
| | acid phosphatase, isozyme alpha) - Homo | | | |
| | sapiens(Human), 157 aa. | | | |
| A53874 | protein-tyrosine-phosphatase(EC 3.1.3.48) | 1 . . . 119 | 107/157(68%) | 2e-54 |
| | isoenzyme AcPI - rat, 157 aa. | 1 . . . 157 | 114/157(72%) | |

PFam analysis predicts that the NOV49a protein contains the domains shown in the Table 49E.

TABLE 49E
Domain Analysis of NOV49a

| Pfam | NOV49a | Identities/Similarities | Expect |
|---|---|---|---|
| Domain | Match Region | for the Matched Region | Value |
| LMWPc | 6 . . . 117 | 46/162(28%) | 4.6e-35 |
| | | 108/162(67%) | |

### Example B

Sequencing Methodology and Identification of NOVX Clone

1. GeneCalling™ Technology: This is a proprietary method of performing differential gene expression profiling between two or more samples developed at CuraGen and described by Shimkets. et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes where ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology: The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary, cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) there then transferred from E.coli into a CuraGen Corporation proprietary yeast strain (disclosed in U.S. Pat. Nos. 6.0,57,101 and 6,083,693, incorporated herein by reference in their entireties).

Gal4-binding domain (Gal4-BD) fusions of a CuraGen Corporation proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

7. Construction of the mammalian expression vector pCEP4/Sec. The oligonucleotide primers. pSec-V5-His Forward (5′-CTCGTC CTCGAG GGT AAG CCT ATC CCT AAC-3′)(SEQ ID NO: 369) and the pSec-V5-His Reverse (5′-CTCGTC GGGCCCCTGATCAGCGGGTTTTAAAC-3′)(SEQ ID NO: 370), were designed to amplify a fragment from the pcDNA3.1-V5His (Invitrogen, Carlsbad, Calif.) expression vector. The PCR product was digested with XhoI and ApaI and ligated into the XhoI/ApaI digested pSecTag2 B vector (Invitrogen, Carlsbad Calif.). The correct structure of the resulting vector, pSecV5His, was verified by DNA sequence analysis. The vector pSecV5His was digested with PmeI and NheI, and the PmeI-NheI fragment was ligated into the BamHI/Klenow and NheI treated vector pCEP4 (Invitrogen, Carlsbad, Calif.). The resulting vector was named as pCEP4/Sec.

Table 50 represents the expression of CG59325-02 in human embryonic kidney 293 cells. A 1.2 kb BamHI-XhoI fragment containing the CG59325-02 sequence was subcloned into BamHI-XhoI digested pCEP4/Sec to generate plasmid 998. The resulting plasmid 998 was transfected into 293 cells using the LipofectaminePlus reagent following the manufacturer's instructions (Gibco/BRL). The cell pellet and supernatant were harvested 72 h post transfection and examined for CG59325-02 expression by Western blot (reducing conditions) using an anti-V5 antibody. Table 50 shows that CG59325-02 is expressed as a 50 kDa protein secreted by 293 cells.

[image]

8. Construction of the mammalian expression vector pCEP4/Sec. The oligonucleotide primers, pSec-V5-His Forward (5′-CTCGTC CTCGAG GGT AAG CCT ATC CCT AAC-3) )(SEQ ID NO: 369) and the pSec-V5-His Reverse (5′-CTCGTC GGGCCCCTGATCAGCGGGTTTAAAC-3′)(SEQ ID NO: 370), were destined to amplify a fragment from the pcDNA-3.1-V5His (Invitrogen, Carlsbad, Calif.) expression vector. The PCR product was digested with XhoI and ApaI and ligated into the XhoI/ApaI digested pSecTag2 B vector (Invitrogen, Carlsbad Calif.). The correct structure of the resulting vector, pSecV5His, was verified by DNA sequence analysis. The vector pSecV5His was digested with PmeI and NheI, and the PmeI-NheI fragment was ligated into the BamHI/Klenow and NheI treated vector pCEP4 (Invitrogen, Carlsbad, Calif.). The resulting vector was named as pCEP4/Sec.

Table 51 represents the CG57209-03 protein secreted by 293 cells. A 1.7 kb BamHI-XhoI fragment containing the CG57209-03 sequence was subcloned into BamHI-XhoI digested pCEP4/Sec to generate plasmid 820. The resulting plasmid 820 was transfected into 293 cells using the LipofectaminePlus reagent following the manufacturer's instructions (Gibco/BRL). The cell pellet and supernatant were harvested 72 h post transfection and examined for CG57209-03 expression by Western blot (reducing conditions) using an anti-V5 antibody. Table 51 shows that CG57209-03 is expressed as a 85 kDa protein secreted by 293 cells.

[image]

### Example C

Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM-® 7700 or an ABI PRISM® 7900 HT Sequence Detection System. Various collections of samples are assembled on the plates, and referred to as Panel 1 (containing normal tissues and cancer cell lines). Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_panel (containing normal tissue and samples from autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1× TaqMan® Universal Master mix (Applied Biosystems; catalog No. 4324020), following the manufacturer's instructions.

Probes and primers ere designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C. maximum primer difference=2° C. probe does not have 5′G, probe Tm must be 10° C. greater than primer Tm, amplicon size 75 bp to 100 bp. The probes and primers selected (see below) were synthesized by Synthegen (Houston, Tex. USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers. 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working faith sscDNA samples, normalized sscDNA was used as described previously for RNA samples. PCR reactions containing one or two sets of probe and primers were set up as described previously, using 1× TaqMan® Universal Master mix (Applied Biosystems; catalog No. 4324020), following the manufacturer's instructions. PCR amplification was performed as follows: 95° C. 10 min, then 40 cycles of 95° C. or 15 seconds, 60° C. for 1 minute. Results were analyzed and processed as described previously.

Panels 1, 1.1, 1.2, and 1.3D

The plates for Panels 1. 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are snidely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

General_screening_panel_v1.4, v1.5 and v1.6

The plates for Panels 1.4, 1.5, and 1.6 include 2 control wells (genomic DNA control and chemistry control) and 94 wells containing cDNA from various samples. The samples in Panels 1.4. 1.5, and 1.6 arc broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panels 1.4, 1.5. and 1.6 are widely available through the American Type Culture Collection (ATCC), a repository for cultured cell lines, and were cultured using the conditions recommended by the ATCC. The normal tissues found on Panels 1.4. 1.5. and 1.6 are comprised of pools of samples derived from all major organ Systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D, 2.2, 2.3 and 2.4

The plates for Panels 2D, 2.2, 2.3 and 2.4 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI) or from Ardais or Clinomics). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI/CHTN/Ardais/Clinomics). Unmatched RNA samples from tissues without malignancy (normal tissues) were also obtained from Ardais or Clinomics. This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition. RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

HASS Panel v 1.0

The HASS panel v 1.0 plates are comprised of 93 cDNA samples and two controls. Specifically. 81 of these samples are derived from cultured human cancer cell lines that had been subjected to serum starvation, acidosis and anoxia for different time periods as well as controls for these treatments. 3 samples of human primary cells. 9 samples of malignant brain cancer (4 medulloblastomas and 5 glioblastomas) and 2 controls. The human cancer cell lines are obtained from ATCC (American Type Culture Collection) and fall into the following tissue groups: breast cancer, prostate cancer, bladder carcinomas, pancreatic cancers and CNS cancer cell lines. These cancer cells are all cultured under standard recommended conditions. The treatments used (serum starvation, acidosis and anoxia) have been previously published in the scientific literature. The primary human cells were obtained from Clonetics (Walkersville, Md.) and were grown in the media and conditions recommended by Clonetics. The malignant brain cancer samples are obtained as part of a collaboration (Henry Ford Cancer Center) and are evaluated by a pathologist prior to CuraGen receiving the samples. RNA was prepared from these samples using the standard procedures. The genomic and chemistry control wells have been described previously.

ARDAIS Panel v 1.0

The plates for ARDAIS panel v 1.0 generally include 2 control wells and 22 test samples composed of RNA isolated from human tissue procured by surgeons working in close cooperation with Ardais Corporation. The tissues are derived from human lung malignancies (lung adenocarcinoma or lung squamous cell carcinoma) and in cases where indicated many malignant samples have “matched margins” obtained from noncancerous lung tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). Unmatched malignant and non-malignant RNA samples from lungs were also obtained from Ardais. Additional information from Ardais provides a gross histopathological assessment of tumor differentiation grade and stage. Moreover, most samples include the original surgical pathology report that provides information regarding the clinical state of the patient.

Panel 3D, 3.1 and 3.2

The plates of Panel 3D, 3.1 and 3.2 are comprised of 94 cDNA samples and two control samples Specifically, 92 of these samples are derived from cultured human cancer cell lines. 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancel, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells ale all cultured under standard recommended conditions and RNA extracted using the standard procedures. The cell lines in panel 3D, 3.1, 3.2, 1, 1.1., 1.2, 1.3D, 1.4, 1.5, and 1.6 are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml. IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using, Ficoll. LAK cells were prepared from these cells by culture in DMEM 5% FCS (Hyclone). 100 μM non essential amino acids (Gibco/Life Technologies. Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% /FCS (Hyclone). 100 μM non essential amino acids (Gibco), 1 mM sodium pyruvate (Gibco), mercaptoethanol 5.5×10⁻⁵M (Gibco), and 10mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples ere obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone). 100 μM non essential amino acids (Gibco). 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using, CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM noni essential amino acids (Gibco). 1 M sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3. ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amilo acids (Gibco), 1 mM sodium pyruvate (Gibco), mercaptoethanol 5.5×10⁻⁵M (Gibco), and 10 mM Hepes (Gibco) and IL.-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential aminio acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs anid 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5.5×10⁵ cells/ml for 8 days, changing the media every 3 days and adjusting the cell concentration to 5.5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential aminio acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5ng,/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

Al_comprehensive panel_v1.0

The plates for AI_comprehensive panel_v 1.0 include two control wells and 89 test samples comprised of cDNA isolated from surgical and postmortem human tissues obtained from the Backus Hospital and Clinomics (Frederick. Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1 anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the Al_comprehensive panel_v1.0 panel, the following abbreviations are used:

Al=Autoimmunity

Syn=Synovial

Normal=No apparent disease

Rep22 /Rep20=individual patients

RA=Rheumatoid arthritis

Backus=From Backus Hospital

OA=Osteoarthritis

(SS)(BA)(MF)=Individual patients

Adj=Adjacent tissue

Match control=adjacent tissues

-M=Male

-F=Female

COPD=Chronic obstructive pulmonary disease

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adiocyte differentiation was induced in donor progenitor cells obtained from Osirus (a division of Clonetics/Bio Wittaker) in triplicate, except for Donor 3U which had only two replicates. Scientists at Clonetics isolated, grew and differentiated human mesenchymal stem cells (HuMSCs) for CuraGen based on the published protocol found in Mark F. Pittener, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, Adipose Midway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubules uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease. Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex). Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions ere chosen to encompass all levels of neurodegeneration in AD. The hippocampus is a region of early and severe neuronal loss in AD the temporal cortex is known to show neurodegeneration in AD after the hippocampus, the parietal cortex shown moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel the following abbreviations are used:

AD=Alzheimer's disease brain: patient was demented and showed AD-like pathology upon autopsy

Control=Control brains; patient not demented, showing no neuropathology

Control (Path)=Control brains; patient not demented but showing sever AD-like pathology

SupTemporal Ctx=Superior Temporal Cortex

Inf Temporal Ctx=Inferior Temporal Cortex

A. CG102071-03: MAP Kinase Phosphatase-like.

Expression of gene CG102071-03 was assessed using the primer-probe set Ag6815, described in Table AA. Results of the RTQ-PCR runs are shown in Tables AB and AC.

TABLE AA

Probe Name Ag6815

| | | | | |
|---|---|---|---|---|
| Primers | Sequences | Length | Start Position | SEQ ID No |

| | | | | |
|---|---|---|---|---|
| Forward | 5′-ttgcacttcttgacatagg-3′ | 119 | 142 | 177 |
| Probe | TET-5′-acctctgcaaggtctgctcgttactat-3′-TAMRA | 27 | 167 | 178 |
| Reverse | 5′-gtcacttcattgggtatcag-3′ | 20 | 229 | 179 |

TABLE AB
General_screening_panel_v1.6

| | Rel. |
|---|---|
| | Exp.(%) |
| | Ag6815, |
| | Run |
| Tissue Name | 278019589 |
| Adipose | 1.4 |
| Melanoma*Hs688(A).T | 6.9 |
| Melanoma*Hs688(B).T | 8.4 |
| Melanoma*M14 | 0.2 |
| Melanoma*LOXIMVI | 11.9 |
| Melanonia*SK-MEL-5 | l4.4 |
| Squamous cell carcinoma SCC-4 | 21.3 |
| Testis Pool | 3 0 |
| Prostate ca.*(bone met) PC-3 | 7.4 |
| Prostate Pool | 2.8 |
| Placenta | 4.9 |
| Uterus Pool | 0.0 |
| Ovarian ca. OVCAR-3 | 31.6 |
| Ovarian ca. SK-OV-3 | 29 7 |
| Ovarian ca. OVCAR-4 | 26.6 |
| Ovarian ca. OVCAR-5 | 32.1 |
| Ovarian ca. IGROV-1 | 27.0 |
| Ovarian ca. OVCAR-8 | 5.7 |
| Ovary | 1.7 |
| Breast ca. MCF-7 | 34.2 |
| Breast ca. MDA-MB-231 | 77.9 |
| Breast ca. BT 549 | 12.2 |
| Breast ca. T47D | 12.1 |
| Breast ca. MDA-N | 0.0 |
| Breast Pool | 3.6 |
| Trachea | 2.8 |
| Lung | 2.0 |
| Fetal Lung | 3.7 |
| Lung ca. NCI-N417 | 4.9 |
| Lung ca. LX-1 | 20.4 |
| Lung ca. NCI-H146 | 2.2 |
| Lung ca. SHP-77 | 0.6 |
| Lung ca. A549 | 35.6 |
| Lung ca. NCI-H526 | 1.6 |
| Lung ca. NCI-H23 | 23.3 |
| Lung ca. NCI-H460 | 3.2 |
| Lung ca. HOP-62 | 16.2 |
| Lung ca. NCI-H1522 | 41.8 |
| Liver | 1.1 |
| Fetal Liver | 2.8 |
| Liver ca. HepG2 | 1.0 |
| Kidney Pool | 2.2 |
| Fetal Kidney | 0.0 |
| Renal ca. 786-0 | 39.8 |
| Renal ca. A498 | 3.9 |
| Renal ca. ACHN | 1.2 |
| Renal ca. UO-31 | 45.7 |
| Renal ca. TK-10 | 76.8 |
| Bladder | 11.3 |
| Gastric ca. (liver met.) NCI-N87 | 53.2 |
| Gastric ca. KATO III | 80.7 |
| Colon ca. SW-948 | 15.6 |
| Colon ca. SW480 | 100.0 |
| Colon ca.*(SW480 met) SW620 | 24.0 |
| Colon ca. HT29 | 25.2 |
| Colon ca. HCT-116 | 30.6 |
| Colon ca. CaCo-2 | 6.6 |
| Colon cancer tissue | 9.6 |
| Colon ca. SW1116 | 18.3 |
| Colon ca. Colo-205 | 6.1 |
| Colon ca. SW-48 | 8.9 |
| Colon Pool | 3.2 |
| Small Intestine Pool | 0.2 |
| Stomach Pool | 0.8 |
| Bone Marrow Pool | 0.9 |
| Fetal Heart | 0.4 |
| Heart Pool | 1.2 |
| Lymph Node Pool | 2.2 |
| Fetal Skeletal Muscle | 1.4 |
| Skeletal Muscle Pool | 0.0 |
| Spleen Pool | 2.5 |
| Thymus Pool | 1.8 |
| CNS cancer(glio/astro) U87-MG | 70.7 |
| CNS cancer(glio/astro) U-118-MG | 14.0 |
| CNS cancer(neuro;met) SK-N-AS | 36.6 |
| CNS cancer(astro) SF-539 | 15.9 |
| CNS cancer(astro) SNB-75 | 40.3 |
| CNS cancer(glio) SNB-19 | 31.0 |
| CNS cancer(glio) SF-295 | 39.0 |
| Brain(Amygdala) Pool | 3.3 |
| Brain(cerebellum) | 2.2 |
| Brain(fetal) | 2.0 |
| Brain(Hippocampus) Pool | 1.8 |
| Cerebral Cortex Pool | 1.0 |
| Brain(Substantia nigra) Pool | 1.0 |
| Brain(Thalamus) Pool | 3.6 |
| Brain(whole) | 0.9 |
| Spinal Cord Pool | 2.7 |
| Adrenal Gland | 4.5 |
| Pituitary gland Pool | 0.6 |
| Salivary Gland | 3.1 |
| Thyroid(female) | 4.0 |
| Pancreatic ca. CAPAN2 | 42.6 |
| Pancreas Pool | 3.1 |

TABLE AC
Panel 4.1D

| | Rel. |
|---|---|
| | Exp.(%) |
| | Ag6815, |
| | Run |
| Tissue Name | 278022637 |
| Secondary Th1 act | 28.7 |
| Secondary Th2 act | 65.5 |
| Secondary Tr1 act | 26.4 |
| Secondary Th1 rest | 1.9 |
| Secondary Th2 rest | 12.9 |
| Secondary Tr1 rest | 9.2 |
| Primary Th1 act | 17.4 |
| Primary Th2 act | 55.1 |
| Primary Tr1 act | 54.7 |
| primary Th1 rest | 0.0 |
| Primary Th2 rest | 4.1 |
| Primary Tr1 rest | 0.7 |
| CD45RA CD4 lymphocyte act | 54.0 |
| CD45RO CD4 lymphocyte act | 39.5 |
| CD8 lymphocyte act | 6.9 |
| Secondary CD8 lymphocyte rest | 0.0 |
| Secondary CD8 lymphocyte act | 6.5 |
| CD4 lymphocyte none | 1.8 |
| 2ry Th1/Th2/Tr1_anti-CD95 CH11 | 10.0 |
| LAK cells rest | 12.2 |
| LAK cells IL-2 | 3.0 |
| LAK cells IL-2 + IL-12 | 0.0 |
| LAK cells IL-2 + IFN gamma | 0.0 |
| LAK cells IL-2 + IL-18 | 2.5 |
| LAK cells PMA/ionomycin | 1.0 |
| NK Cells IL-2 rest | 72.7 |
| Two Way MLR 3 day | 20.3 |
| Two Way MLR 5 day | 6.0 |
| Two Way MLR 7 day | 3.6 |
| PBMC rest | 1.8 |
| PBMC PWM | 9.6 |
| PBMC PHA-L | 10.5 |
| Ramos(B cell) none | 17.2 |
| Ramos(B cell) ionomycin | 78.5 |
| B lymphocytes PWM | 3.4 |
| B lymphocytes CD40L and IL-4 | 25.2 |
| EOL-1 dbcAMP | 25.3 |
| EOL-1 dbcAMP PMA/ionomycin | 7.6 |
| Dendritic cells none | 8.8 |
| Dendritic cells LPS | 6.3 |
| Dendritic cells anti-CD40 | 13.2 |
| Monocytes rest | 7.1 |
| Monocytes LPS | 45.7 |
| Macrophages rest | 8.4 |
| Macrophages LPS | 21.0 |
| HUVEC none | 23.8 |
| HUVEC starved | 27.9 |
| HUVEC IL-1beta | 40.9 |
| HUVEC IFN gamma | 38.4 |
| HUVEC TNF alpha + IFN gamma | 24.0 |
| HUVEC TNF alpha + IL4 | 21.0 |
| HUVEC IL-11 | 10.5 |
| Lung Microvascular EC none | 86.5 |
| Lung Microvascular EC TNFalpha + IL-1beta | 27.0 |
| Microvascular Dermal EC none | 5.5 |
| Microsvasular Dermal EC TNFalpha + | 0.0 |
| IL-1beta | |
| Bronchial epithelium TNFalpha + | 27.2 |
| IL1beta | |
| Small airway epithelium none | 0.0 |
| Small airway epithelium TNFalpha + | 5.8 |
| IL-1beta | |
| Coronery artery SMC rest | 63.7 |
| Coronery artery SMC TNFalpha + IL-1beta | 32.1 |
| Astrocytes rest | 12.9 |
| Astrocytes TNFalpha + IL-1beta | 7.1 |
| KU-812(Basophil) rest | 0.0 |
| KU-812(Basophil) PMA/ionomycin | 0.0 |
| CCD1106(Keratinocytes) none | 100.0 |
| CCD1106(Keratinocytes) TNFalpha + | 9.7 |
| IL-1beta | |
| Liver cirrhosis | 5.6 |
| NCI-H292 none | 0.9 |
| NCI-H292 IL-4 | 6.5 |
| NCI-H292 IL-9 | 3.8 |
| NCI-H292 IL-13 | 1.9 |
| NCI-H292 IFN gamma | 0.0 |
| HPAEC none | 0.0 |
| HPAEC TNFalpha + IL-1beta | 18.8 |
| Lung fibroblast none | 11.0 |
| Lung fibroblast TNF alpha + IL-1beta | 22.5 |
| Lung fibroblast IL-4 | 9.5 |
| Lung fibroblast IL-9 | 17.6 |
| Lung fibroblast IL-13 | 12.9 |
| Lung fibroblast IFN gamma | 27.2 |
| Dermal fibroblast CCD1070 rest | 57.4 |
| Dermal fibroblast CCD1070 TNF alpha | 94.0 |
| Dermal fibroblast CCD1070 IL-1 beta | 26.2 |
| Dermal fibroblast IFN gamma | 6.6 |
| Dermal fibroblast IL-4 | 11.7 |
| Dermal Fibroblasts rest | 8.5 |
| Neutrophils TNFa + LPS | 2.6 |
| Neutrophils rest | 5.5 |
| Colon | 3.1 |
| Lung | 0.0 |
| Thymus | 0.0 |
| Kidney | 25.9 |

CNS_neurodegeneration_v1.0 Summary: Ag6815 Results from one experiment with this gene are not included. The amp plot indicates that there were experimental difficulties with this run.

General_screening_panel_v1.6 Summary: Ag6815 Highest expression of this gene is seen in a colon cancer cell line (CT=28.7). This gene is widely expressed in this panel, with prominent levels of expression in all cancer cell lines, including brain, pancreatic, renal, gastric, lung, breast, ovarian, and melanoma cancer cell lines. This expression profile suggests a role for this gene product in cell survival and proliferation. Modulation of this gene product may be useful in the treatment of cancer.

Among tissues with metabolic function, this gene is expressed at low but significant levels in adipose, adrenal gland, pancreas, thyroid, fetal skeletal muscle, and adult and fetal liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

This gene is also expressed at loss but significant levels in the CNS, including the hippocampus, thalamus, substantia nigra, amygdala, cerebellum and cerebral cortex. Therefore, therapeutic modulation of the expression or function of this gene may be useful in the treatment of neurologic disorders. Such as Alzheimer's disease, Parkinson's disease, schizophrenia, multiple sclerosis, stroke and epilepsy.

Panel 4.1D Summary: Ag6815 Highest expression is seen in untreated keratinocytes. (CT=31.3). Moderate levels of expression are seen in several untreated or resting cell types, including NK cells, coronary artery SMCs, lung microvascular endothelial cells, as well as in activated primary and secondary T cells. In addition, this gene is expressed at low but significant levels in many other samples on this pane. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

B. CG102734-01 and CG102734-02: RAS-Related Protein RAB-4A.

Expression of gene CG102734-01 and CG102734-02 was assessed using the primer-probe set Ag4213, described in Table BA. Results of the RTQ-PCR runs are shown in Tables BB and BC.

TABLE BA

Probe Name Ag4213

| | | | | |
|---|---|---|---|---|
| Primers | Sequences | Length | Start Position | SEQ ID No: |

| | | | | |
|---|---|---|---|---|
| Forward | 5′-gaaaagasaatttsgsgtgttc-3′ | 22 | 870 | 180 |
| Probe | 5′-ccagtcaaagtggcacagcaaatcat-3′-TAMRA | 26 | 898 | 181 |
| Reverse | 5′-catctaacggtgttgtccattt-3′ | 22 | 936 | 182 |

TABLE BB
General_screening_panel_v1.4

| | Rel. |
|---|---|
| | Exp.(%) |
| | Ag4213, |
| | Run |
| Tissue Name | 213323527 |
| Adipose | 6.1 |
| Melanoma*Hs688(A).T | 24.0 |
| Melanoma*Hs688(B).T | 32.5 |
| Melanoma*M14 | 26.8 |
| Melanoma*LOXIMVI | 12.1 |
| Melanoma*SK-MEL-5 | 18.8 |
| Cell carcinoma SCC-4 | 17.2 |
| Testis Pool | 10.6 |
| Prostate ca.(bone met) PC-3 | 52 5 |
| Prostate Pool | 21.8 |
| Placenta | 9.5 |
| Uterus Pool | 6.1 |
| Ovarian ca. OVCAR-3 | 49.3 |
| Ovarian ca. SK-OV-3 | 100.0 |
| Ovarian ca. OVCAR-4 | 12.3 |
| Ovarian ca. OVCAR-5 | 40.1 |
| Ovarian ca. IGROV-1 | 26.2 |
| Ovarian ca. OVCAR-8 | 24.7 |
| Ovary | 19.8 |
| Breast ca. MCF-7 | 62.0 |
| Breast ca. MDA-MB-231 | 34.6 |
| Breast ca. BT 549 | 47.6 |
| Breast ca. T47D | 97.9 |
| Breast ca. MDA-N | 0.0 |
| Breast Pool | 22.1 |
| Trachea | 37.9 |
| Lung | 10.7 |
| Fetal Lung | 16.7 |
| Lung ca. NCI-N417 | 2.8 |
| Lung ca. LX-1 | 46.7 |
| Lung ca. NCI-H146 | 8.2 |
| Lung ca. SHP-77 | 20.4 |
| Lung ca. A549 | 37.9 |
| Lung ca. NCI-H526 | 6.0 |
| Lung ca. NCI-H23 | 73.2 |
| Lung ca. NCI-H460 | 55.5 |
| Lung ca. HOP-62 | 20.6 |
| Lung ca. NCI-H522 | 60.7 |
| Liver | 4.1 |
| Fetal Liver | 36.6 |
| Liver ca. HepG2 | 81.2 |
| Kidney Pool | 34.2 |
| Fetal Kidney | 13.6 |
| Renal ca. 786-0 | 17.6 |
| Renal ca. A498 | 4.4 |
| Rcnal ca. ACHN | 21.3 |
| Renal ca. UO-31 | 7.4 |
| Renal ca. TK-10 | 47.0 |
| Bladder | 30.8 |
| Gastric ca.(liver met.) NCI-N87 | 38.2 |
| Gastric ca. KATO III | 49.0 |
| Colon ca SW-948 | 16.5 |
| Colon ca. SW480 | 49.7 |
| Colon ca*(SW480 met)SW620 | 30.8 |
| Colon ca. HT29 | 31.9 |
| Colon ca HCT-116 | 69.7 |
| Colon ca. CaCo-2 | 39.5 |
| Colon cancer tissue | 23.2 |
| Colon ca. SW1116 | 4.7 |
| Colon ca. Colo-205 | 23.8 |
| Colon ca. SW-48 | 23.8 |
| Colon Pool | 18.4 |
| Small Intestine Pool | 16.5 |
| Stomach Pool | 17.7 |
| Bone Marrow Pool | 8.0 |
| Fetal Heart | 6.2 |
| Heart Pool | 10.1 |
| Lymph Node Pool | 19.5 |
| Fetal Skeletal Muscle | 5.9 |
| Skeletal Muscle Pool | 16.6 |
| Spleen Pool | 7.1 |
| Thymus Pool | 11.4 |
| CNS cancer(glio/astro)U87-MG | 9.7 |
| CNS cancer(glio/astro)U-118-MG | 0.0 |
| CNS cancer(neuro;met)SK-N-AS | 35.4 |
| CNS cancer(astro)SF-539 | 9.8 |
| CNS cancer(astro)SNB-75 | 41.8 |
| CNS cancer(glio)SNB-19 | 24.1 |
| CNS cancer(glio)SF-295 | 32.1 |
| Brain(Amygdala)Pool | 25.9 |
| Brain(cerebellum) | 35.6 |
| Brain(fetal) | 41.2 |
| Brain(Hippocampus)Pool | 21.3 |
| Cerebral Cortex Pool | 19.8 |
| Brain(Substantia nigra)Pool | 24.0 |
| Brain(Thalamus)Pool | 39.8 |
| Brain(whole) | 38.2 |
| Spinal Cord Pool | 20.9 |
| Adrenal Gland | 14.1 |
| Pituitary gland Pool | 4.0 |
| Salivary Gland | 26.6 |
| Thyroid(Female) | 13.1 |
| Pancreatic ca. CAPAN2 | 40.3 |
| Pancreas Pool | 24.7 |

TABLE BC
Panel 5 Islet

| | Rel. |
|---|---|
| | Exp.(%) |
| | Ag4213, |
| | Run |
| Tissue Name | 174269009 |
| 97457_Patient-02go_adipose | 9.1 |
| 97476_Patient-07sk_skeletal | 15.1 |
| muscle | |
| 97477_Patient-07ut_uterus | 22.4 |
| 97478_Patient-07pl_placenta | 9.7 |
| 99167_Bayer Patient 1 | 40.9 |
| 97482_Patient-08ut_uterus | 17.3 |
| 97483_Patient-08pl_placenta | 8.4 |
| 97486_Patient-09sk_skeletal | 6.2 |
| muscle | |
| 97487_Patient-09ut_uterus | 17.8 |
| 97488_Patient-09pl_placenta | 7.9 |
| 97492_Patient-10ut_uterus | 25.2 |
| 97493_Patient-10pl_placenta | 26.1 |
| 97495_Patient-11go_adipose | 7.4 |
| 97496_Patient-11sk_skeletal | 18.8 |
| muscle | |
| 97497_Patient-11ut_uterus | 1.0 |
| 97498_Patient-11pl_placenta | 8.8 |
| 97500_Patient-12go_adipose | 10.7 |
| 97501_Patient-12sk_skeletal | 70.2 |
| muscle | |
| 97502_Patient-12ut_uterus | 46.3 |
| 97503_Patient-12pl_placenta | 10.6 |
| 94721_Donor 2 U - | 19.6 |
| A_Mesenchymal Stem Cells | |
| 94722_Donor 2 U - | 16.4 |
| B_Mesenchymal Stem Cells | |
| 94723_Donor 2 U - | 26.2 |
| C_Mesenchymal Stem Cells | |
| 94709_Donor 2 AM - A_adipose | 21.6 |
| 94710_Donor 2 AM - B_adipose | 19.2 |
| 94711_Donor 2 AM - C_adipose | 9.6 |
| 94712_Donor 2 AD - A_adipose | 23.2 |
| 94713_Donor 2 AD - B_adipose | 35.8 |
| 94714_Donor 2 AD - C_adipose | 21.2 |
| 94742_Donor 3 U - A_Mesenchymal Stem | 8.2 |
| Cells | |
| 94743_Donor 3 U - B_Mesenchymal Stem | 17.2 |
| Cells | |
| 94730_Donor 3 AM - A_adipose | 20.2 |
| 94731_Donor 3 AM - B_adipose | 10.7 |
| 94732_Donor 3 AM - C_adipose | 9.8 |
| 94733_Donor 3 AD - A_adipose | 23.5 |
| 94734_Donor 3 AD - B_adipose | 13.1 |
| 94735_Donor 3 AD - C_adipose | 0.9 |
| 77138_Liver_HepG2untreated | 100.0 |
| 73556_Heart_Cardiac stromal cells | 6.9 |
| (primary) | |
| 81735_Small Intestine | 20.6 |
| 72409_Kidney_Proximal Convoluted | 9.2 |
| Tubule | |
| 82685_Small intestine_Duodenum | 24.8 |
| 90650_Adrenal_Adrenocortical adenoma | 9.5 |
| 72410_Kidney_HRCE | 20.3 |
| 72411_Kidney_HRE | 15.7 |
| 73139_Uterus_Uterine smooth muscle cells | 3.7 |

General_screening_panel_v1.4 Summary: Ag4213 Highest expression of this gene is seen in an ovarian cancer cell line (CT=26). This gene is widely expressed in this panel, with high to moderate expression seen in all cancer cell lines on this panel, including brain, colon, gastric, lung, breast, ovarian, and melanoma cancer cell lines. This expression profile suggests a role for this gene product in cell survival and proliferation. Modulation of this gene product may be useful in the treatment of cancer.

Among tissues with metabolic function, this gene is expressed at moderate levels in pituitary, adipose, adrenal gland, pancreas, thyroid, and adult and fetal skeletal muscle, heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes. In addition, this gene is expressed at much higher levels in fetal tissue (CT=27.5) when compared to expression in the adult counterpart (CT=30.5). Thus, expression of this gene may be used to differentiate between the fetal and adult source of this tissue.

This gene is also expressed at moderate levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 5 Islet Summary: Ag4213 Highest expression of this gene is seen in a liver derived cell line (CT=29). In addition, moderate levels of expression are seen in metabolic tissues, including placenta, skeletal muscle and human islet cells. Rab4 has been shown to participate both in the intracellular retention of glucose transporter containing vesicles and in the insulin signaling pathway leading to glucose transporter translocation. (Le Marchand-Brustel, J Recept Signal Transduct Res 1999 January-July,19(1-4):217-28). Thus the expression of this putative Rab4 protein in tissues with metabolic function suggests that therapeutic modulation of the expression or function of this gene product may be of use in the treatment of insulin resistance, and associated obesity and type II diabetes.

C. CG 112785-01: G PCR.

Expression of gene CG112785-01 was assessed using the primer-probe set Ag4463, described in Table CA.

TABLE CA

Probe Name Ag4463

| Primers | Sequences | Length | Start Position | SEQ ID No |
|---|---|---|---|---|
| Forward | 5′-atcctaacccctttgtcacatt-3′ | 22 | 1085 | 183 |
| Probe | TET-5′-tgcttgatggttttattcctttccaca-3′-TAMRA | 27 | 1115 | 184 |
| Reverse | 5′-ggcataacaaagaagcaattca-3′ | 22 | 1151 | 185 |

CNS_neurodegeneration_v1.0 Summary: Ag4463 Expression of this gene is low/undetectable in all samples on this panel (CTs>35). (Data not shown.) The amp plot indicates that there is a high probability of a probe failure.

General_screening_panel_v1.4 Summary: Ag4463 Expression of this gene is low/undetectable in all samples on this panel (CTs>35). (Data not Shown.) The amp plot indicates that there is a high probability of a probe failure.

Panel 4.1D Summary: Ag4463 Expression of this gene is low/undetectable in all samples on this panel (CTs>35). (Data not shown.) The amp plot indicates that there is a high probability of a probe failure.

General Oncology Screening panel_v_{—}2.4 Summary: Ag4463 Expression of this gene is low/undetectable in all samples on this panel (CTs>35). (Data not shown.) The amp plot indicates that there is a high probability of a probe failure.

D. CG116818-02: Pyruvate Carboxylase Precursor.

Expression of gene CG116818-02 was assessed using, the primer-probe set Ag4745, described in Table DA.

TABLE DA

Probe Name Ag4745

| | | | | |
|---|---|---|---|---|
| Primers | Sequences | Length | Start Position | SEQ ID No |

| | | | | |
|---|---|---|---|---|
| Forward | 5′-gccaaggagaacaacgtagat-3′ | 21 | 405 | 186 |
| Probe | TET-5′-accctggctacgggttcctttctgag-3′-TAMRA | 26 | 433 | 187 |
| Reverse | 5′-ctgccaccactttgatgtctat-3′ | 22 | 471 | 188 |

CNS_neurodegeneration_v1.0 Summary: Ag4745 Expression of this gene is low/undetectable in all samples on this panel (CTs>35). (Data not shown.)

General_screening panel_v1.4 Summary: Ag4745 Expression of this (gene is low/undetectable in all samples on this panel (CTs>35). (Data not shown.)

Panel 4.1D Summary: Ag4745 Expression of this gene is low/undetectable in all samples on this panel (CTs>35). (Data not shown.)

Panel 5 Islet Summary: Ag4745 Expression of this gene is low/undetectable in all samples on this panel (CTs>35). (Data not shown.)

E. CG117653-02: Human ATP Binding Cassette ABCG1 (ABC8).

Expression of gene CG117653-02 was assessed using the primer-probe set Ag4881. described in Table EA. Results of the RTQ-PCR runs are shown in Tables EB and EC.

TABLE EA

Probe Name Ag4881

| | | | | |
|---|---|---|---|---|
| Primers | Sequences | Length | Start Position | SEQ ID No |

| | | | | |
|---|---|---|---|---|
| Forward | 5′-accaagaagqtcttgagcaact-3′ | 22 | 1360 | 189 |
| Probe | TET-5′-cttctccatgctgttcctcatgttcg-3′-TAMRA | 26 | 1395 | 190 |
| Reverse | 5′-caggggaaatgtcagaacagta-3′ | 22 | 191 | |

TABLE EB
General_screening_panel_v1.5

| | Rel. |
|---|---|
| | Exp.(%) |
| | Ag4881, |
| | Run |
| Tissue Name | 228806996 |
| Adipose | 5.6 |
| Melanoma*Hs688(A).T | 0.1 |
| Melanoma*Hs688(B).T | 0.0 |
| Melanoma*M14 | 0.0 |
| Melanoma*LOXIMVI | 0.0 |
| Melanoma*SK-MEL-5 | 0.4 |
| Squamous cell carcinoma SCC-4 | 1.0 |
| Testis Pool | 2.5 |
| Prostate ca.*(bone met)PC-3 | 10.2 |
| Prostate Pool | 2.5 |
| Placenta | 18.9 |
| Uterus Pool | 10.7 |
| Ovarian ca. OVCAR-3 | 4.6 |
| Ovarian ca. SK-OV-3 | 1.1 |
| Ovarian ca. OVCAR-4 | 0.8 |
| Ovarian ca. OVCAR-5 | 36.1 |
| Ovarian ca. IGROV-1 | 3.2 |
| Ovarian ca. OVCAR-8 | 1.4 |
| Ovary | 3.0 |
| Breast ca. MCF-7 | 15.5 |
| Breast ca. MDA-MB-231 | 2.1 |
| Breast ca. BT 549 | 0.0 |
| Breast ca. T47D | 3.4 |
| Breast ca. MDA-N | 0.1 |
| Breast Pool | 5.4 |
| Trachea | 16.8 |
| Lung | 1.6 |
| Fetal Lung | 55.9 |
| Lung ca. NCI-N417 | 0.0 |
| Lung ca. LX-1 | 28.1 |
| Lung ca. NCI-H146 | 7.8 |
| Lung ca. SHP-77 | 14.7 |
| Lung ca. A549 | 12.2 |
| Lung ca. NCI-H526 | 9.3 |
| Lung ca. NCI-H23 | 54.3 |
| Lung ca. NCI-H460 | 15.0 |
| Lung ca. HOP-62 | 4.0 |
| Lung ca. NCI-H522 | 17.8 |
| Liver | 1.5 |
| Fetal Liver | 5.4 |
| Liver ca. HepG2 | 0.0 |
| Kidney Pool | 4.4 |
| Fetal Kidney | 5.8 |
| Renal ca. 786-0 | 0.1 |
| Renal ca. A498 | 5.6 |
| Renal ca. ACHN | 0.0 |
| Renal ca. UO-31 | 2.1 |
| Renal ca. TK-10 | 0.0 |
| Bladder | 12.9 |
| Gastric ca.(liver met.)NCI-N87 | 58.6 |
| Gastric ca. KATO III | 6.7 |
| Colon ca. SW-948 | 0.1 |
| Colon ca. SW480 | 6.2 |
| Colon ca.(SW480 met)SW620 | 9.0 |
| Colon ca. HT29 | 5.1 |
| Colon ca. HCT-116 | 1.3 |
| Colon ca. CaCo-2 | 1.1 |
| Colon cancer tissue | 34.2 |
| Colon ca. SW1116 | 0.5 |
| Colon ca. Colo-205 | 8.0 |
| Colon ca. SW-48 | 3.9 |
| Colon Pool | 3.9 |
| Small Intestine Pool | 4.6 |
| Stomach Pool | 9.1 |
| Bone Marrow Pool | 1.8 |
| Fetal Heart | 7.5 |
| Heart Pool | 2.3 |
| Lymph Node Pool | 3.2 |
| Fetal Skeletal Muscle | 2.8 |
| Skeletal Muscle Pool | 15.2 |
| Spleen Pool | 41.5 |
| Thymus Pool | 20.9 |
| CNS cancer(glio/astro)U87-MG | 0.0 |
| CNS cancer(glio/astro)U-118-MG | 0.0 |
| CNS cancer(neuro;met)SK-N-AS | 0.1 |
| CNS cancer(astro)SF-539 | 0.0 |
| CNS cancer(astro)SNB-75 | 1.5 |
| CNS cancer(glio)SNB-19 | 2.9 |
| CNS cancer(glio)SF-295 | 43.2 |
| Brain(Amygdala)Pool | 18.7 |
| Brain(cerebellum) | 100.0 |
| Brain(fetal) | 18.8 |
| Brain(Hippocampus)Pool | 15.7 |
| Cerebral Cortex Pool | 8.0 |
| Brain(Substantia nigra)Pool | 15.0 |
| Brain(Thalamus)Pool | 23.7 |
| Brain(whole) | 23.8 |
| Spinal Cord Pool | 7.3 |
| Adrenal Gland | 56.6 |
| Pituitary gland Pool | 7.7 |
| Salivary Gland | 6.3 |
| Thyroid(female) | 3.8 |
| Pancreatic ca. CAPAN2 | 1.5 |
| Pancreas Pool | 7.1 |

TABLE EC
Oncology cell_line_screening_panel_v3.1

| | Rel. |
|---|---|
| | Exp(%) |
| | Ag4881, |
| | Run |
| Tissue Name | 225052577 |
| Daoy Medulloblastoma/Cerebellum | 0.5 |
| TE671 Medulloblastom/Cerebellum | 2.6 |
| D283 Med | 0.5 |
| Medulloblastoma/Cerebellum | |
| PFSK-1 Primitive | 7.3 |
| Neuroectodermal/Cerebellum | |
| XF-498_CNS | 2.9 |
| SNB-78_CNS/glioma | 1.0 |
| SF-268_CNS/glioblastoma | 0.0 |
| T98G_Glioblastoma | 0.0 |
| SK-N-SH_Neuroblastoma | 0.2 |
| (Metastasis) | |
| SF-295_CNS/glioblastoma | 2.0 |
| Cerebellum | 38.4 |
| Cerebellum | 40.6 |
| NCI-H292_Mucoepidermoid lung ca. | 29.1 |
| DMS-114_Small cell lung cancer | 0.2 |
| DMS-79_Small cell lung | 9.9 |
| cancer/neuroendocrine | |
| NCI-H146_Small cell lung | 15.4 |
| cancer/neuroendocrine | |
| NCI-H526_Small cell lung | 31.4 |
| cancer/neuroendocrine | |
| NCI-N417_Small cell lung | 0.2 |
| cancer/neuroendocrine | |
| NCI-H82_Small cell lung | 0.8 |
| cancer/neuroendocrine | |
| NCI-H157_Squamous cell lung | 0.0 |
| cancer(metastasis) | |
| NCI-H1155_Large cell lung | 100.0 |
| cancer/neuroendocrine | |
| NCI-H1299_Large cell lung | 0.5 |
| cancer/neuroendocrine | |
| NCI-H727_Lung carcinoid | 61.1 |
| NCI-UMC-11_Lung carcinoid | 4.4 |
| LX-1_Small cell lung cancer | 5.0 |
| Colo-205_Colon cancer | 12.3 |
| KM12_Colon cancer | 0.1 |
| KM20L2_Colon cancer | 4.2 |
| NCI-H716_Colon cancer | 23.7 |
| SW-48_Colon adenocarcinoma | 8.1 |
| SW1116_Colon adenocarcinoma | 0.3 |
| LS 174T_Colon adenocarcinoma | 1.0 |
| SW-948_Colon adenocarcinoma | 0.0 |
| SW-480_Colon adenocarcinoma | 0.2 |
| NCI-SNU-5_Gastric ca | 2 7 |
| KATO III_Stomach | 2.0 |
| NCI-SNU-16_Gastric ca. | 0.0 |
| NCI-SNU-1_Gastric ca. | 0.7 |
| RF-1_Gastric adenocarcinoma | 8.6 |
| RF-48_Gastric adenocarcinoma | 12.9 |
| MKN-45_Gastric ca. | 2.0 |
| NCI-N87_Gastric ca. | 1.8 |
| OVCAR-5_Ovarian ca. | 4.3 |
| RL95-2_Uterine carcinoma | 4.4 |
| HelaS3_Cervical adenocarcinoma | 12.9 |
| Ca Ski_Cervical epidermoid carcinoma | 0.0 |
| (metastasis) | |
| ES-2_Ovarian clear cell carcinoma | 0.0 |
| Ramos/6h stim_Stimulated with | 1.1 |
| PMA/ionomycin 6h | |
| Ramos/14h stim_Stimulated with | 2.3 |
| PMA/ionomycin 14h | |
| MEG-01_Chronic myelogenous | 1.5 |
| leukemia(megokaryoblast) | |
| Raji_Burkitt's lymphoma | 0.2 |
| Daudi_{—Burkitt's lymphoma} | 1.5 |
| U266_B-cell plasmacytoma/mycloma | 6.4 |
| CA46_Burkitt's lymphoma | 5.3 |
| RL_non-Hodgkin's B-cell lymphoma | 0.0 |
| JM1_pre-B-cell lymphoma/leukemia | 5.9 |
| Jurkat_T cell leukemia | 31.0 |
| TF-1_Erythroleukemia | 0.0 |
| HUT 78_T-cell lymphoma | 3.7 |
| U937_Histiocytic lymphoma | 0.0 |
| KU-812_Myelogenous leukemia | 0.5 |
| 769-P_Clear cell renal ca. | 0.0 |
| Caki-2_Clear cell renal ca. | 0.4 |
| SW 839_Clear cell renal ca. | 0.0 |
| G401_Wilms' tumor | 0.3 |
| Hs766T_Pancreatic ca.(LN metastasis) | 27.5 |
| CAPAN-1_Pancreatic adenocarcinoma | 1.7 |
| (liver metastasis) | |
| SU86.86_Pancreatic carcinoma(liver | 6.0 |
| metastasis) | |
| BxPC-3_Pancreatic adenocarcinoma | 0.0 |
| HPAC_Pancreatic aclenocarcinoma | 7.5 |
| MIA PaCa-2_Pancreatic ca. | 0.0 |
| CFPAC-1_Pancreatic ductal | 3.3 |
| adenocarcinoma | |
| PANC-1_Pancreatic epithelioid ductal | 0.7 |
| ca. | |
| T24_Bladder ca.(transitional cell) | 19.1 |
| 5637_Bladder ca. | 4.7 |
| HT-1197 Bladder ca. | 8.2 |
| UM-UC-3_Bladder ca.(transitional | 0.0 |
| cell) | |
| A204_Rhabdomyosarcoma | 0.1 |
| HT-1080_Fibrosarcoma | 0.0 |
| MG-63_Osteosarcoma(bone) | 1.1 |
| SK-LMS-1_Leiomyosarcoma(vulva) | 0 2 |
| SJRH30_Rhabdomyosarcoma(met to | 0.0 |
| bone marrow) | |
| A431_Epidermoid ca. | 0.4 |
| WM266-4_Melanoma | 0.0 |
| DU 145_Prostate | 0.1 |
| MDA-MB-468_Breast | 0.6 |
| adenocarcinoma | |
| SSC-4_Tongue | 0.4 |
| SSC-9_Tongue | 0.0 |
| SSC-15_Tongue | 3.7 |
| CAL 27_Squamous cell ca. of tongue | 2.6 |

General_screening_panel_v1.5 Summary: Ag4881 Highest expression of this gene is seen in the cerebellum (CT=27.5). Moderate levels of expression are also seen in all regions of the CNS examined. Moderate to low levels of expression of this gene are also seen in metabolic tissues, including pancreas, thyroid, adrenal, pituitary, adipose, fetal and adult heart, skeletal muscle, and liver. This gene encodes a member of the ATP-binding cassette (ABC) transporter family. The ABC superfamily comprises of myriad transmembrane proteins involved in the transport of vitamins, peptides, steroid hormones, ions, sugars, and amino acids (ref. 1). Known genetic diseases resulting from dysfunctional ABC transporters include cystic fibrosis, Zellweger syndrome, adrenoleukodystrophy, multidrug resistance, Stargardt macular dystrophy, Tangier disease (TD) and familial HDL deficiency (FHA) (ref. 2, 3). Recently, it has been shown that functional loss of ABCA1, a transporter belonging to ABCA subfamily, in mice causes severe placental malformation, aberrant lipid distribution, and kidney glomeruloniephritis, as well as, high-density lipoprotein cholesterol deficiency (ref 3). This gene is expressed in large number of the normal tissue used in this panel. In analogy to ABCA1, this gene may also play a wider role in lipid metabolism, renal inflammation, and cardiovascular disease and CNS disorders.

References.

1. Higgins C F. (1992) Annu Rev Cell Biol 8:67-113 PMID: 1282354

2. Decottignies A, Goffeau A. (1997) Nat Genet 15(2):137-45. PMID: 9020838

3. Christiansen-Weber T A, Voland J R, Wu Y, Ngo K, Roland B L, Nguyen S, Peterson P A, Fung-Leung W P.(2000) Am J Pathiol 2000 September,157(3):1017-29

Oncology_cell line_screening_panel_v3.1 Summary: Ag4881 Highest levels of expression are seen in a lung cancer cell line (CT=27.5). Moderate levels of expression are also seen in the cerebellum, in agreement with Panel 1.5. This expression in the cerebellum suggests that this gene product may be a useful and specific target of drugs for the treatment of CNS disorders that have this brain region as the site of pathology, such as autism and the ataxias.

F. CG119674-02: Orphan Neurotransmitter Transporter NTT5.

Expression of gene CG119674-02 was assessed using the primer-probe set Ag7022, described in Table FA.

What is claimed is:

1. An isolated polypeptide comprising the mature form of an amino acid sequenced selected from the group consisting of SEQ ID NO: 2n, wherein n is an integer between 1 and 88.

2. An isolated polypeptide comprising an amino acid sequence selected from the group consisting of SEQ ID NO: 2n, wherein n is an integer between 1 and 88.

3. An isolated polypeptide comprising an amino acid sequence which is at least 95% identical to an amino acid sequence selected from the group consisting of SEQ ID NO: 2n, wherein n is an integer between 1 and 88.

4. An isolated polypeptide, wherein the polypeptide comprises an amino acid sequence comprising one or more conservative substitutions in the amino acid sequence selected from the group consisting of SEQ ID NO: 2n, wherein n is an Integer between 1 and 88.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim 1, wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide, thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing, said polypeptide to said agent; and
(b) determining, whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide, wherein a change in the activity of said polypeptide in said test animal relative to said control animal indicates the test compound is a modulator activity of or latency or predisposition to, a pathology associated with the polypeptide of claim 1.

15. The method of claim 14, %,hereini said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which Such treatment or prevention is desired in an amount sufficient to treat or prevent the pathology in the subject.

18. The method of claim 17, wherein the subject is a human.

19. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO: 2n, wherein n is an integer between 1 and 88 or a biologically active fragment thereof.

20. An isolated nucleic acid molecule comprising a nucleic acid sequence selected from the group consisting of SEQ ID NO: 2n−1 wherein n is an integer between 1 and 88.

21. The nucleic acid molecule of claim 20, wherein the nucleic acid molecule is naturally occurring.

22. A nucleic acid molecule, wherein the nucleic acid molecule differs by a single nucleotide from a nucleic acid sequence selected from the group consisting of SEQ ID NO: 2n−1 wherein n is all integer between 1 and 88.

23. An isolated nucleic acid molecule encoding the mature form of a polypeptide having an amino acid sequence selected from the group consisting of SEQ ID NO: 2n wherein n is an integer between 1 and 88.

24. An isolated nucleic acid molecule comprising a nucleic acid selected from the group consisting of 2n-1, wherein n is an integer between 1 and 88.

25. The nucleic acid molecule of claim 20 wherein said nucleic acid molecule hybridizes under stringent conditions to the nucleotide sequence selected from the group consisting of SEQ ID NO: 2n−1, wherein n is an integer between 1 and 88, or a complement of said nucleotide sequence.

26. A vector comprising the nucleic acid molecule of claim 20.

27. The vector of claim 26, further comprising a promoter operably linked to said nucleic acid molecule.

28. A cell comprising the vector of claim 26.

29. An antibody that immunospecifically binds to the polypeptide of claim 1.

30. The antibody of claim 29, wherein the antibody is a monoclonal antibody.

31. The antibody of claim 29, wherein the antibody is a humanized antibody.

32. A method for determining the presence or amount of the nucleic acid molecule of claim 20 in a sample, the method comprising:
(a) providing said sample;
(b) introducing, said sample to a probe that binds to said nucleic acid molecule; and
(c) determining the presence or amount of said probe bound to said nucleic acid molecule,
thereby determining the presence or amount of the nucleic acid molecule in said sample.

33. The method of claim 32 wherein presence or amount of the nucleic acid molecule is used as a marker for cell or tissue type.

34. The method of claim 33 wherein the cell or tissue type is cancerous.

35. A method for determining the presence of or predisposition to a disease associated with altered levels of expression of the nucleic acid molecule of claim 20 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.

36. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO: 2n−1, wherein n is an integer between 1 and 88.

37. The method of claim 36 wherein the cell is a bacterial cell.

38. The method of claim 36 wherein the cell is an insect cell.

39. The method of claim 36 wherein the cell is a yeast cell.

40. The method of claim 36 wherein the cell is a mammalian cell.

41. A method of producing the polypeptide of claim 2, the method comprising culturing a cell under conditions that lead to expression of the polypeptide, wherein said cell comprises a vector comprising an isolated nucleic acid molecule comprising a nucleic acid sequence selected from the group consisting of SEQ ID NO: 2n−1, wherein n is an integer between 1 and 88.

42. The method of claim 41 wherein the cell is a bacterial cell.

43. The method of claim 41 wherein the cell is an insect cell.

44. The method of claim 41 wherein the cell is a yeast cell.

45. The method of claim 41 wherein the cell is a mammalian cell.
